(19) United States
(12) Patent Application Publication
(10) Pub. No.: US 2024/0245673 A1
(43) Pub. Date: Jul. 25, 2024

(54) Papain-like protease (PLpro) inhibitors

(71) Applicant: Pfizer Inc., New York, NY (US)

(72) Inventors: James Matthew Frick, Washington, DC (US); Michelle Renee Garnsey, Medford, MA (US); Brian Stephen Gerstenberger, Cambridge, MA (US); Xinjun Hou, Winchester, MA (US); Magdalena Korczynska, Somerville, MA (US); Alpha Albert Lee, Cambridge, MA (US); Luong Tien Nguyen, Kansas City, MO (US); Usa Reilly, West Haven, CT (US); Matthew Christopher Robinson, Cambridge, MA (US); Alexandria Paige Taylor, East Greenwich, RI (US); Thomas Reynold Vargo, Stow, MA (US); Lei Zhang, Auburnadale, MA (US)

(73) Assignee: Pfizer Inc., New York, NY (US)

(21) Appl. No.: 18/532,424

(22) Filed: Dec. 7, 2023

Related U.S. Application Data
(60) Provisional application No. 63/594,119, filed on Oct. 30, 2023.
(60) Provisional application No. 63/505,758, filed on Jun. 2, 2023.
(60) Provisional application No. 63/495,810, filed on Apr. 13, 2023.
(60) Provisional application No. 63/386,748, filed on Dec. 9, 2022.

(51) Int. Cl. A61K 31/4709; A61K 31/415; A61K 31/4178; A61K 31/4192; A61K 31/427; A61K 31/454; A61K 31/496; A61K 31/5377; A61K 45/06; C07D 207/34; C07D 215/38; C07D 231/12; C07D 249/06; C07D 401/04; C07D 401/12; C07D 401/14; C07D 403/12; C07D 403/14; C07D 405/12; C07D 405/14; C07D 409/04; C07D 413/04; C07D 413/12; C07D 417/12; C07D 417/14; C07D 471/10; C07D 487/04; C07D 487/08; C07D 495/04

(52) U.S. Cl. CPC A61K 31/4709; A61K 31/415; A61K 31/4178; A61K 31/4192; A61K 31/427; A61K 31/454; A61K 31/496; A61K 31/5377; A61K 45/06; C07D 207/34; C07D 215/38; C07D 231/12; C07D 249/06; C07D 401/04; C07D 401/12; C07D 401/14; C07D 403/12; C07D 403/14; C07D 405/12; C07D 405/14; C07D 409/04; C07D 413/04; C07D 413/12; C07D 417/12; C07D 417/14; C07D 471/10; C07D 487/04; C07D 487/08; C07D 495/04

(57) ABSTRACT

The invention relates to compounds of Formula (I)

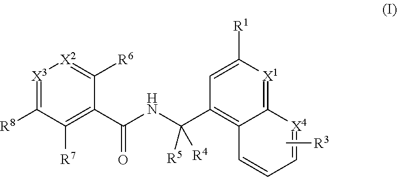

and pharmaceutically acceptable salts thereof as defined in the description; to their use in medicine; to compositions containing them; to processes for their preparation; and to intermediates used in such processes. The compounds of Formula (I) may inhibit the activity of papain-like protease (PLpro) and may be useful in the treatment of viral infections, in particular viral infections associated with PLpro activity and/or expression such as coronaviruses infections.

### BACKGROUND OF THE INVENTION

The present invention relates to novel compounds which are inhibitors of the papain-like protease (PLpro). The invention also relates to the preparation of these compounds and intermediates used in their preparation, compositions containing the compounds of the invention, and their use including their use to treat viral infections, in particular viral infections characterized with papain-like protease activity and/or expression such as coronaviruses infections.

PLpro is a cysteine protease with a papain-like fold. PLpro is conserved across many coronaviruses, including SARS-CoV, MERS-CoV and SARS-CoV-2 (ACS Infect. Dis., 2020, 6, 8, 2099-2109). These viruses can cause severe acute respiratory tract infections, including the COVID-19 pandemic.

Viruses harboring PLpro, such as coronaviruses, are known in the literature to be causal agents of historic outbreaks and pandemics, for example the SARS outbreak in 2003 (N. Engl. J. Med., 2003; 349, 2431), the MERS-CoV outbreak in 2012 (Annu. Rev. Med. 2017. 68:387-99), and the COVID-19 pandemic beginning in 2020 (N. Engl. J. Med., 2020, 382, 727; Nat. Rev. Microbiol., 2022, 20, 270). They are also known in the literature to be likely to cause future pandemics (J. Infect. Dis., 2022, jiac296).

PLpro is responsible for processing cleavage sites in the viral polyproteins to produce functional units, which in turn assemble to execute RNA synthesis and other viral functions. PLpro also modulates host innate immune pathways, through deubiquitination and delSGylation activities. The enzymatic activity of PLpro is therefore essential to viral replication and evading host immune response (Nature, 2020, 587, 657-662). Numerous publications have evidenced that if PLPro can be selectively inhibited, it could prevent viral replication and be used in the treatment of viral infections (J. Med. Chem. 2022, 65, 4, 2940; Cell Chemical Biology, 2021, 28, 855-865; ACS Cent. Sci. 2021, 7, 7, 1245).

PLpro inhibitors have already been reported, for example in the aforementioned publications and in WO 2010/022355, WO 2022/192665, WO 2022/070048, WO 2022/169891 or WO 2022/189810. However, there remains a need for new compounds having an improved therapeutic profile as PLpro inhibitors, namely an improved activity.

### SUMMARY OF THE INVENTION

The present invention provides, in part, compounds of Formula (I) and pharmaceutically acceptable salts thereof. Such compounds may inhibit the activity of the papain-like protease (PLpro) and may be useful in the treatment, prevention, suppression and amelioration of viral infections, in particular viral infections characterized with PLpro activity and/or expression such as coronaviruses infections, and infections caused by other nidoviruses.

Also provided are pharmaceutical compositions, comprising the compounds or salts of the invention, alone or in combination with other therapeutic agents, which may provide greater clinical benefit. Such additional therapeutic agents include, but are not limited to, viral RNA polymerase inhibitors, Mpro inhibitors, nucleoside inhibitors, host factor inhibitors, another PLpro inhibitors or metabolism boosting agents that leads to reduction in virus replication or host response that may contribute to greater clinical benefit.

The present invention also provides, in part, methods for preparing such compounds, pharmaceutically acceptable salts and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention there is provided a compound of Formula (I):

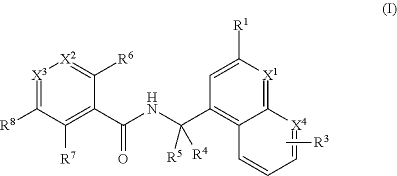

or a pharmaceutically acceptable salt thereof, wherein:
X¹ is N or CR²;
X² is N or CH;
X³ is N or CR⁹ provided that X² and X³ cannot be both N;
X⁴ is CH or N, provided that X¹ and X⁴ are not N at the same time and X⁴ can only be N when R⁶ is —CH₃, R⁷ is H, R⁸ is H, X³ is CR⁹ and R⁹ is —(CR^{g}R^{h})—O—(CR^{g}R^{h})R¹²;
R² is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R³ is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R¹ is selected from the group consisting of -L¹-L²-L³-Cyc¹, C₁-C₆ alkyl, C₁-C₆ aminoalkyl, C₁-C₆ cyanoalkyl, C₁-C₆ alkyl-C(═O)—N(R^{a})R^{b}, —N(R^{a})R^{b}, —N(R^{a})(C₁-C₆ alkoxy-C₁-C₆ alkyl), —N(R^{a})(C₁-C₆ alkyl-C(═O)—C₁-C₆ alkyl), —N(R^{a})(C(═O)—C₁-C₆ alkyl), —N(R^{a})(O₁—C₆ alkyl-C(═O)—N(R^{c})R^{d}), —N(R^{a})(C₁-C₆ alkyl-SO₂—N(R^{c})R^{d}), —O—C₁-C₆ hydroxyalkyl, —O—(C₁-C₆ alkoxy-C₁-C₆ alkyl);
L¹, L² and L³ are each either absent or independently selected from the group consisting of O, NR^{a}, C(═O) and C₁-C₃ alkylene;
Cyc¹ is selected from the group consisting of C₆-C₁₀ aryl, 5-10 membered heteroaryl, C₃-C₈cycloalkyl, 3-10 membered heterocycloalkyl, C₃-C₈ cycloalkenyl and 3-8 membered heterocycloalkenyl, each optionally substituted by one, two or three R¹⁰;
R^{a}, R^{b}, R^{c} and R^{d} are each independently selected at each occurrence from the group consisting of H and C₁-C₄ alkyl;
R⁴ and R⁵ are each independently selected from the group consisting of H and C₁-C₄ alkyl, C₁-C₄ aminoalkyl and C₁-C₄ alkoxy-C₁-C₄ alkyl, or alternatively R⁴ and R⁵ together with the carbon atom to which they are attached form a C₃-C₆ cycloalkyl optionally substituted by one R¹⁰ or a 4-8 membered heterocycloalkyl optionally substituted by one R¹⁰;
R⁶ is selected from the group consisting of H, halogen, cyano, nitro, C₁-C₆ alkyl, —C₁-C₆ fluoroalkyl, C₁-C₆ aminoalkyl, C₁-C₆ alkoxy-C₁-C₆ alkyl, —OR¹², —SR¹², C₁-C₃ alkyl-C(═O)—N(R^{e})—N(R^{f})—C(═O)—CH═CH—C(═O)—O—C₁-C₆ alkyl, C₁-C₃ alkyl-C(═O)—N(R^{e})—N(R^{f})—C(═O)—CH═CH—O—C₁-C₆ alkyl, C₁-C₃ alkyl-C(═O)—N(R^{e})—N(R^{f})—C(═O)-oxiran-C(═O)—O—C₁-C₆ alkyl, C₁-C₃ alkyl-C(═O)—N(R^{e})—N(R^{f})—C(═O)-oxiran-C(═O)—OH and C₁-C₃ alkyl-C(═O)—N(R^{e})—N(R)—C(═O)—CH═CH—C(═O)—N(R¹¹)R¹²;
R^{e} and R^{f} are each independently selected at each occurrence from the group consisting of H and C₁-C₄ alkyl;
R⁷ and R⁸ are each independently selected from the group consisting of H, halogen, hydroxy, C₁-C₆ alkyl, C₁-C₆ alkoxy-C₁-C₆ alkyl, 4-10 membered heterocycloalkyl, —C₁-C₆ alkyl-NR¹⁴R¹⁵, —NHR¹⁶, —N(R¹³)—C(═O)—R¹⁶, and —C(═O)—NR¹⁴R¹⁵, wherein any said 4-10 membered heterocycloalkyl is optionally substituted with one, two or three R¹⁰;
R⁹ is selected from the group consisting of H, halogen, hydroxy, cyano, C₁-C₆ alkyl, C₁-C₆ alkoxy-C₁-C₆ alkyl, C₁-C₆ alkyl-C(═O)—OH, —C(═O)—O—C₁-C₆ alkyl, —C(═O)—N(R¹¹)R¹², —C(═O)—N(R¹¹)—C₁-C₃ alkyl-R¹², —C(═O)—N(R¹¹)—(CR^{g}R^{h})—R¹², —(CR^{g}R^{h})—CH(OH)—(CR^{g}R^{h})—R¹², —(CR^{g}R^{h})—CH(OCH₃)—(CR^{g}R^{h})—R¹², —(CR^{g}R^{h})—N(R¹¹)—(CR^{g}R^{h})—R¹², —(CR^{g}R^{h})—O—(CR^{g}R^{h})—R¹², —(CR^{g}R^{h})—S—(CR^{g}R^{h})—R¹², —(CR^{g}R^{h})—S(═O)—(CR^{g}R^{h})—R¹², —(CR^{g}R^{h})—SO₂—(CR^{g}R^{h})—R¹², —SO₂—N(R¹¹)—(CR^{g}R^{h})—R¹², —C₁-C₃ alkyl-C(═O)—N(R¹¹)R¹², —(CR^{g}R^{h})—C(═O)—N(R¹¹)R¹², —(CR^{g}R^{h})—SO₂—N(R¹¹)R¹², —C₁-C₃ alkyl-C(═O)—N(R¹¹)—C₁-C₃ alkyl-R¹², —N(R¹¹)R¹², —N(R¹¹)—C₁-C₃ alkyl-R¹², —O—C₁-C₃ alkyl-R¹², —N(R¹¹)—C(═O)—N(R¹³)—R¹², —C₁-C₃ alkyl-N(R¹¹)R¹², —C₁-C₃ alkyl-N(R¹¹)—C₁-C₃ alkyl-R¹², —C₁-C₃ alkyl-N(R¹¹)—C(═O)—R¹², —(CR^{g}R^{h})—N(R¹¹)—(CR^{g}R^{h})—R¹², —(CR^{g}R^{h})—N(R¹¹)—C(═O)—R¹², —C₁-C₃ alkyl-N(R¹¹)—C(═O)—C₁-C₃ alkyl-R¹² and —(CR^{g}R^{h})—OR¹²;

Each of R^{g} and R^{h} is independently selected from the group consisting of H, cyano, halogen, hydroxy, C₁-C₆ alkyl, C₁-C₆ fluoroalkyl, C₁-C₆ aminoalkyl, C₁-C₆ alkoxy, C₁-C₆ hydroxyalkyl, —C₁-C₆ alkoxy-C₁-C₆ alkyl, or alternatively, R^{g} and R^{h} together with the carbon atom to which they are attached form a C₃-C₆ cycloalkyl or a 4-8 membered Heterocycloalkyl, each optionally substituted by one R¹⁰;
with the proviso that at least one of R⁷, R⁸ and R⁹ is H and that R⁶, R⁷, R⁸ and R⁹ are not all H at the same time;

Each R¹⁰ is independently selected at each occurrence from the group consisting of cyano, nitro, oxo, 1-(1-cyanocyclopropyl)methyl, halogen, C₁-C₆ alkyl, C₁-C₆ fluoroalkyl, C₁-C₆ aminoalkyl, —C₁-C₆ alkyl-R¹², C₁-C₆ alkoxy, C₁-C₆ alkoxy-C₁-C₆ alkyl, C₂-C₆ alkenyl, C₂-C₆ alkynyl, C₃-C₆ cycloalkyl, 3-8 membered heterocycloalkyl, —COOH, —C(═O)—O—C₁-C₆ alkyl, —C(═O)—N(R¹¹)R¹², —N(R¹¹)R¹², —C₁-C₃ alkyl-N(R¹¹)R¹², —C₁-C₃ alkyl-C(═O)—N(R¹¹)R¹², —(C(CH₃)₂)—C(═O)—N(R¹¹)R¹², —N(R¹¹)C(═O)R¹², —C₁-C₃ alkyl-N(R¹¹)C(═O)R¹², —S(O)R¹², —S(O)₂R¹², —C₁-C₃ alkyl-S(O)₂R¹², —S(O)₂N(R¹¹)R¹², —N(R¹³)S(O)₂N(R¹¹)R¹², —N(R¹³)S(O)₂R¹², —OR¹², —C₁-C₃ alkyl-OR¹² and —SR¹²;

Each R¹¹ is independently selected from H, —SO₂CH₃, —C(═O)CH₃ and C₁-C₆ alkyl;

Each R¹² is independently selected from H, C₁-C₆ alkyl, C₁-C₆ alkoxy-C₁-C₆ alkyl, C₁-C₆ aminoalkyl, di(C₁-C₃ alkyl)amino-C₁-C₆ alkyl, C₁-C₆ fluoroalkyl, C₃-C₈ cycloalkyl, 4-8 membered heterocycloalkyl, phenyl and 5-6 membered heteroaryl, wherein said C₃-C₈ cycloalkyl, 4-8 membered heterocycloalkyl, phenyl and 5-6 membered heteroaryl are each optionally substituted by one or two R¹⁷;

Or alternatively R¹¹ and R¹² together form a 4-8 membered heterocycloalkyl;

Each R¹³ is independently selected from H and C₁-C₆ alkyl;

Each R¹⁴ and R¹⁵ is independently selected from the group consisting of H, C₁-C₆ alkyl, C₁-C₆ fluoroalkyl, C₁-C₆ aminoalkyl, —SO₂—C₁-C₆ alkyl and C₁-C₆ alkoxy-C₁-C₆ alkyl;

Each R¹⁶ is a 3-8 membered heterocycloalkyl optionally substituted with one, two or three R¹⁰; and

Each R¹⁷ is independently selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl, C₁-C₆ alkoxy and C₃-C₆ cycloalkyl.

Described below are embodiments of the invention, where for convenience Embodiment 1 (E1) is identical to the embodiment of Formula (I) provided above. It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

E1 A compound of Formula (I) or a pharmaceutically acceptable salt thereof, as defined above.

E2 A compound of embodiment E1 or a pharmaceutically acceptable salt thereof, wherein:
X¹ is N or CR²;
X² is N or CH;
X³ is N or CR⁹ provided that X² and X³ cannot be both N;
R² is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R³ is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R¹ is selected from the group consisting of -L¹-L²-L³-Cyc¹, C₁-C₆ alkyl, C₁-C₆ aminoalkyl, C₁-C₆ cyanoalkyl, C₁-C₆ alkyl-C(═O)—N(R^{a})R^{b}, —N(R^{a})R^{b}, —N(R^{a})(C₁-C₆ alkoxy-C₁-C₆ alkyl), —N(R^{a})(C₁-C₆ alkyl-C(═O)—C₁-C₆ alkyl), —N(R^{a})(C(═O)—C₁-C₆ alkyl), —N(R^{a})(C₁-C₆ alkyl-C(═O)—N(R^{c})R^{d}), —N(R^{a})(C₁-C₆ alkyl-SO₂—N(R^{c})R^{d}), —O—C₁-C₆ hydroxyalkyl, —O—(C₁-C₆ alkoxy-C₁-C₆ alkyl);
L¹, L² and L³ are each either absent or independently selected from the group consisting of O, NR^{a}, C(═O) and C₁-C₃ alkylene;
Cyc¹ is selected from the group consisting of C₆-C₁₀ aryl, 5-10 membered heteroaryl, C₃-C₈cycloalkyl, 3-10 membered heterocycloalkyl, C₃-C₈ cycloalkenyl and 3-8 membered heterocycloalkenyl, each optionally substituted by one, two or three R¹⁰;
R^{a}, R^{b}, R^{c} and R^{d} are each independently selected at each occurrence from the group consisting of H and C₁-C₄ alkyl;
R⁴ and R⁵ are each independently selected from the group consisting of H and C₁-C₄ alkyl, C₁-C₄ aminoalkyl and C₁-C₄ alkoxy-C₁-C₄ alkyl, or alternatively R⁴ and R⁵ together with the carbon atom to which they are attached form a C₃-C₆ cycloalkyl optionally substituted by one R¹⁰ or a 4-8 membered heterocycloalkyl optionally substituted by one R¹⁰;
R⁶ is selected from the group consisting of H, halogen, cyano, nitro, C₁-C₆ alkyl, —C₁-C₆ fluoroalkyl, C₁-C₆ aminoalkyl, C₁-C₆ alkoxy-C₁-C₆ alkyl, —OR¹², —SR¹², C₁-C₃ alkyl-C(═O)—N(R^{e})—N(R^{f})—C(═O)—CH═CH—C(═O)—O—C₁-C₆ alkyl, C₁-C₃ alkyl-C(═O)—N(R^{e})—N(R^{f})—C(═O)—CH═CH—O—C₁-C₆ alkyl, C₁-C₃ alkyl-C(═O)—N(R^{e})—N(R^{f})—C(═O)-oxiran-C(═O)—O—C₁-C₆ alkyl, C₁-C₃ alkyl-C(═O)—N(R^{e})—N(R^{f})—C(═O)-oxiran-C(═O)—OH and C₁-C₃ alkyl-C(═O)—N(R^{e})—N(R)—C(═O)—CH═CH—C(═O)—N(R¹¹)R¹²;
R^{e} and R^{f} are each independently selected at each occurrence from the group consisting of H and C₁-C₄ alkyl;
R⁷ and R⁸ are each independently selected from the group consisting of H, halogen, hydroxy, C₁-C₆ alkyl, C₁-C₆ alkoxy-C₁-C₆ alkyl, 4-10 membered heterocycloalkyl, —C₁-C₆ alkyl-NR¹⁴R¹⁵, —NHR¹⁶, —N(R¹³)—C(═O)—R¹⁶, and —C(═O)—NR¹⁴R¹⁵, wherein any said 4-10 membered heterocycloalkyl is optionally substituted with one, two or three R¹⁰;
R⁹ is selected from the group consisting of H, halogen, hydroxy, cyano, C₁-C₆ alkyl, C₁-C₆ alkoxy-C₁-C₆ alkyl, C₁-C₆ alkyl-C(═O)—OH, —C(═O)—O—C₁-C₆ alkyl, —C(═O)—N(R¹¹)R¹², —C(═O)—N(R¹¹)—C₁-C₃ alkyl-R¹², —C(═O)—N(R¹¹)—(CR^{g}R^{h})—R¹², —(CR^{g}R^{h})—N(R¹¹)—(CR^{g}R^{h})—R¹², —(CR^{g}R^{h})—O—(CR^{g}R^{h})—R¹², —(CR^{g}R^{h})_S—(CR^{g}R^{h})—R¹², —(CR^{g}R^{h})—S(═O)—(CR^{g}R^{h})—R¹², —(CR^{g}R^{h})—SO₂—(CR^{g}R^{h})—R¹², —SO₂—N(R¹¹)—(CR^{g}R^{h})—R¹², C₁-C₃ alkyl-C(═O)—N(R¹¹)R¹², —(CR^{g}R^{h})—C(═O)—N(R¹¹)R¹², —(CR^{g}R^{h})—SO₂—N(R¹¹)R¹², —C₁-C₃ alkyl-C(═O)—N(R¹¹)—C₁-C₃ alkyl-R¹², —N(R¹¹)R¹², —N(R¹¹)—C₁-C₃ alkyl-R¹², —O—C₁-C₃ alkyl-R¹², —N(R¹¹)—C(═O)—N(R¹³)—R¹², —C₁-C₃ alkyl-N(R¹¹)R¹², —C₁-C₃ alkyl-N(R¹¹)—C₁-C₃ alkyl-R¹², —C₁-C₃ alkyl-N(R¹¹)—C(═O)—R¹², —(CR^{g}R^{h})—N(R¹¹)—(CR^{g}R^{h})—R¹², —(CR^{g}R^{h})—N(R¹¹)—C(═O)—R¹² and —C₁-C₃ alkyl-N(R¹¹)—C(═O)—C₁-C₃ alkyl-R¹²;

Each of R^{g} and R^{h} is independently selected from the group consisting of H, cyano, halogen, hydroxy, C₁-C₆ alkyl, C₁-C₆ fluoroalkyl, C₁-C₆ aminoalkyl, C₁-C₆ alkoxy, C₁-C₆ hydroxyalkyl, —C₁-C₆ alkoxy-C₁-C₆ alkyl, or alternatively, R^{g} and R^{h} together with the carbon atom to which they are attached form a C₁-C₆ cycloalkyl or a 4-8 membered Heterocycloalkyl, each optionally substituted by one R¹⁰;
with the proviso that at least one of R⁷, R⁸ and R⁹ is H and that R⁶, R⁷, R⁸ and R⁹ are not all H at the same time;

Each R¹⁰ is independently selected at each occurrence from the group consisting of cyano, nitro, oxo, 1-(1-cyanocyclopropyl)methyl, halogen, C₁-C₆ alkyl, C₁-C₆ fluoroalkyl, C₁-C₆ aminoalkyl, —C₁-C₆ alkyl-R¹², C₁-C₆ alkoxy, C₁-C₆ alkoxy-C₁-C₆ alkyl, C₂-C₆ alkenyl, C₂-C₆ alkynyl, C₃-C₆ cycloalkyl, 3-8 membered heterocycloalkyl, —COOH, —C(═O)—O—C₁-C₆ alkyl, —C(═O)—N(R¹¹)R¹², —N(R¹¹)R¹², —C₁-C₃ alkyl-N(R¹¹)R¹², —N(R¹¹)C(═O)R¹², —C₁-C₃ alkyl-N(R¹¹)C(═O)R¹², —S(O)R¹², —S(O)₂R¹², —S(O)₂N(R¹¹)R¹², —N(R¹³)S(O)₂N(R¹¹)R¹², —N(R¹³)S(O)₂R¹², —OR¹², —C₁-C₃ alkyl-OR¹² and —SR¹²;

Each R¹¹ is independently selected from H, —SO₂CH₃, —C(═O)CH₃ and C₁-C₆ alkyl;

Each R¹² is independently selected from H, C₁-C₆ alkyl, C₁-C₆ alkoxy-C₁-C₆ alkyl, C₁-C₆ aminoalkyl, C₁-C₆ fluoroalkyl, C₃-C₈ cycloalkyl, 4-8 membered heterocycloalkyl, phenyl and 5-6 membered heteroaryl, wherein said C₃-C₈ cycloalkyl, 4-8 membered heterocycloalkyl, phenyl and 5-6 membered heteroaryl are each optionally substituted by one or two R¹⁷;

Or alternatively R¹¹ and R¹² together form a 4-8 membered heterocycloalkyl;

Each R¹³ is independently selected from H and C₁-C₆ alkyl;

Each R¹⁴ and R¹⁵ is independently selected from the group consisting of H, C₁-C₆ alkyl, C₁-C₆ fluoroalkyl, C₁-C₆ aminoalkyl, —SO₂—C₁-C₆ alkyl and C₁-C₆ alkoxy-C₁-C₆ alkyl;

Each R¹⁶ is a 3-8 membered heterocycloalkyl optionally substituted with one, two or three R¹⁰; and

Each R¹⁷ is independently selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy.

E3 A compound of embodiment E1 or E2 or a pharmaceutically acceptable salt thereof, wherein:
X¹ is N or CR²;
X² is N or CH;
X³ is CR⁹;
R² is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R³ is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R¹ is selected from the group consisting of -L¹-L²-L³-Cyc¹, C₁-C₆ alkyl, C₁-C₆ aminoalkyl, C₁-C₆ cyanoalkyl, C₁-C₆ alkyl-C(═O)—N(R^{a})R^{b}, —N(R^{a})R^{b}, —N(R^{a})(C₁-C₆ alkoxy-C₁-C₆ alkyl), —N(R^{a})(C₁-C₆ alkyl-C(═O)—C₁-C₆ alkyl), —N(R^{a})(C(═O)—C₁-C₆ alkyl), —N(R^{a})(C₁-C₆ alkyl-C(═O)—N(R^{c})R^{d}), —N(R^{a})(C₁-C₆ alkyl-SO₂—N(R^{c})R^{a}), —O—C₁-C₆ hydroxyalkyl, —O—(C₁-C₆ alkoxy-C₁-C₆ alkyl);
L¹, L² and L³ are each either absent or independently selected from the group consisting of O, NR^{a}, C(═O) and C₁-C₃ alkylene;
Cyc¹ is selected from the group consisting of C₆-C₁₀ aryl, 5-10 membered heteroaryl, C₃-C₈cycloalkyl, 3-10 membered heterocycloalkyl, C₃-C₈ cycloalkenyl and 3-8 membered heterocycloalkenyl, each optionally substituted by one, two or three R¹⁰;
R^{a}, R^{b}, R^{c} and R^{d} are each independently selected at each occurrence from the group consisting of H and C₁-C₄ alkyl;
R⁴ and R⁵ are each independently selected from the group consisting of H and C₁-C₄ alkyl, C₁-C₄ aminoalkyl and C₁-C₄ alkoxy-C₁-C₄ alkyl, or alternatively R⁴ and R⁵ together with the carbon atom to which they are attached form a C₃-C₆ cycloalkyl optionally substituted by one R¹⁰ or a 4-8 membered heterocycloalkyl optionally substituted by one R¹⁰;
R⁶ is selected from the group consisting of H, halogen, cyano, nitro, C₁-C₆ alkyl, —C₁-C₆ fluoroalkyl, C₁-C₆ aminoalkyl, C₁-C₆ alkoxy-C₁-C₆ alkyl, —OR¹², —SR¹², C₁-C₃ alkyl-C(═O)—N(R^{e})—N(R^{f})—C(═O)—CH═CH—C(═O)—O—C₁-C₆ alkyl and C₁-C₃ alkyl-C(═O)—N(R^{e})—N(R)—C(═O)—CH═CH—C(═O)—N(R¹¹)R¹²;
R^{e} and R^{f} are each independently selected at each occurrence from the group consisting of H and C₁-C₄ alkyl;
R⁷ and R⁸ are each independently selected from the group consisting of H, halogen, hydroxy, C₁-C₆ alkyl, C₁-C₆ alkoxy-C₁-C₆ alkyl, 4-10 membered heterocycloalkyl, —C₁-C₆ alkyl-NR¹⁴R¹⁵, —NHR¹⁶, —N(R¹³)—C(═O)—R¹⁶ and —C(═O)—NR¹⁴R¹⁵, wherein any said 4-10 membered heterocycloalkyl is optionally substituted with one, two or three R¹⁰;
R⁹ is selected from the group consisting of H, halogen, hydroxy, C₁-C₆ alkyl, C₁-C₆ alkoxy-C₁-C₆ alkyl, —C(═O)—O—C₁-C₆ alkyl, —C(═O)—N(R¹¹)R¹², —C(═O)—N(R¹¹)—C₁-C₃ alkyl-R¹², —C₁-C₃ alkyl-C(═O)—N(R¹¹)R¹², —C₁-C₃ alkyl-C(═O)—N(R¹¹)—C₁-C₃ alkyl-R¹², —N(R¹¹)R¹², —N(R¹¹)—C₁-C₃ alkyl-R¹², —C₁-C₃ alkyl-N(R¹¹)R¹², —C₁-C₃ alkyl-N(R¹¹)—C₁-C₃ alkyl-R¹², —C₁-C₃ alkyl-N(R¹¹)—C(═O)—R¹² and —C₁-C₃ alkyl-N(R¹¹)—C(═O)—C₁-C₃ alkyl-R¹²,
with the proviso that at least one of R⁷, R⁸ and R⁹ is H and that R⁶, R⁷, R⁸ and R⁹ are not all H at the same time;

Each R¹⁰ is independently selected at each occurrence from the group consisting of cyano, nitro, oxo, 1-(1-cyanocyclopropyl)methyl, halogen, C₁-C₆ alkyl, C₁-C₆ fluoroalkyl, C₁-C₆ aminoalkyl, —C₁-C₆ alkyl-R¹², C₁-C₆ alkoxy, —C₁-C₆ alkoxy-C₁-C₆ alkyl, C₂-C₆ alkenyl, C₂-C₆ alkynyl, C₃-C₆ cycloalkyl, 3-8 membered heterocycloalkyl, —COOH, —C(═O)—O—C₁-C₆ alkyl, —C(═O)—N(R¹¹)R¹², —N(R¹¹)R¹², —C₁-C₃ alkyl-N(R¹¹)R¹², —N(R¹¹)C(═O)R¹², —C₁-C₃ alkyl-N(R¹¹)C(═O)R¹², —S(O)R¹², —S(O)₂R¹², —S(O)₂N(R¹¹)R¹², —N(R¹³)S(O)₂N(R¹¹)R¹², —N(R¹³)S(O)₂R¹², —OR¹², —C₁-C₃ alkyl-OR¹² and —SR¹²;

Each R¹¹ is independently selected from H and C₁-C₆ alkyl;

Each R¹² is independently selected from H, C₁-C₆ alkyl, C₁-C₆ alkoxy-C₁-C₆ alkyl, C₁-C₆ aminoalkyl, C₁-C₆ fluoroalkyl, C₃-C₈ cycloalkyl, 4-8 membered heterocycloalkyl, phenyl and 5-6 membered heteroaryl, wherein said C₃-C₈ cycloalkyl, 4-8 membered heterocycloalkyl, phenyl and 5-6 membered heteroaryl are each optionally substituted by one or two R¹⁷;

Or alternatively R¹¹ and R¹² together form a 4-8 membered heterocycloalkyl;

Each R¹³ is independently selected from H and C₁-C₆ alkyl;

Each R¹⁴ and R¹⁵ is independently selected from the group consisting of H, C₁-C₆ alkyl, C₁-C₆ fluoroalkyl, C₁-C₆ aminoalkyl, —SO₂—C₁-C₆ alkyl and C₁-C₆ alkoxy-C₁-C₆ alkyl;

Each R¹⁶ is a 3-8 membered heterocycloalkyl optionally substituted with one, two or three R¹⁰; and

Each R¹⁷ is independently selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy.

E4 A compound of any one of embodiments E1 to E3 or a pharmaceutically acceptable salt thereof, wherein X² is CH.

E5 A compound of any one of embodiments E1 to E4, or a pharmaceutically acceptable salt thereof, wherein X¹ is N and X⁴ is CH.

E6 A compound of any one of embodiments E1 to E4, or a pharmaceutically acceptable salt thereof, wherein X¹ is CH, C(OH) or C(OCH₃).

E7 A compound of embodiment E6, or a pharmaceutically acceptable salt thereof, wherein X¹ is CH.

E8 A compound of any one of embodiments E1 to E7, or a pharmaceutically acceptable salt thereof, wherein R³ is H.

E9 A compound of any one of embodiments E1 to E8, or a pharmaceutically acceptable salt thereof, wherein R⁴ and R⁵ are each independently selected from the group consisting of H and C₁-C₄ alkyl or R⁴ and R⁵ together with the carbon atom to which they are attached form a C₃-C₆ cycloalkyl.

E10 A compound of embodiment E9, or a pharmaceutically acceptable salt thereof, wherein R⁴ and R⁵ are each independently selected from the group consisting of H and methyl or R⁴ and R⁵ together with the carbon atom to which they are attached form a cyclopropyl.

E11 A compound of embodiment E10, or a pharmaceutically acceptable salt thereof, wherein R⁴ is H and R⁵ is methyl.

E12 A compound of embodiment E10, or a pharmaceutically acceptable salt thereof, wherein R⁴ and R⁵ together with the carbon atom to which they are attached form a cyclopropyl.

E13 A compound of any one of embodiments E1 to E12, or a pharmaceutically acceptable salt thereof, wherein R⁶ is methyl and R⁷ is H.

E14 A compound of any one of embodiments E1 to E3 having Formula (Ia1):

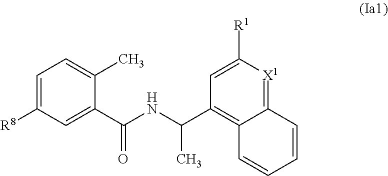

or a pharmaceutically acceptable salt thereof, wherein X¹ is CH, C(OH), C(OCH₃) or N.

E15 A compound of embodiment E14 which is of formula:

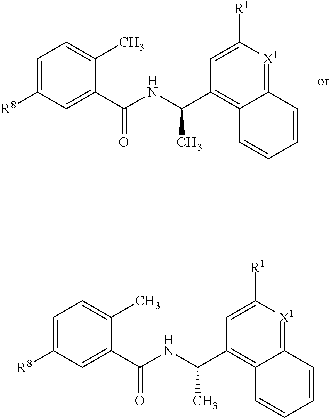

pharmaceutically acceptable salt thereof.

E16 A compound of any one of embodiments E1 to E3 having Formula (Ia2):

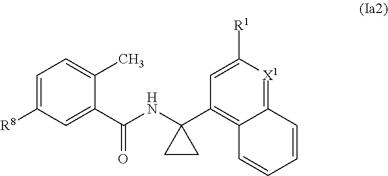

or a pharmaceutically acceptable salt thereof, wherein X¹ is CH or N.

E17 A compound of any one of embodiments E14 to E16, or a pharmaceutically acceptable salt thereof, wherein R⁸ is selected from the group consisting of —C₁-C₆ alkyl-NR¹⁴R¹⁵, —C(═O)—NR¹⁴R¹⁵, 4-10 membered heterocycloalkyl optionally substituted with one or two R¹⁰ and —N(R¹³)—C(═O)—R¹⁶; R¹³ is H or C₁-C₆ alkyl; R¹⁴ and R¹⁵ are each independently H, C₁-C₆ alkyl or —SO₂—C₁-C₆ alkyl; R¹⁶ is a 3-8 membered heterocycloalkyl optionally substituted with one or two R¹⁰; and R¹⁰ is oxo, amino, hydroxy or C₁-C₆ alkyl.

E18 A compound of embodiment E17, or a pharmaceutically acceptable salt thereof, wherein R⁸ is —C(═O)NH₂, —CH₂NH₂, —CH₂NHSO₂CH₃, —NH—C(═O)-6 membered heterocycloalkyl, 6-10 membered heterocycloalkyl, —CH₂NH(CH₃) or —N(CH₃)—C(═O)-6 membered heterocycloalkyl, wherein any said heterocycloalkyl is optionally substituted with one or two R¹⁰ each independently selected from the group consisting of oxo, amino, hydroxy and methyl.

E19 A compound of any one of embodiments E1 to E3 having the formula (Ib1):

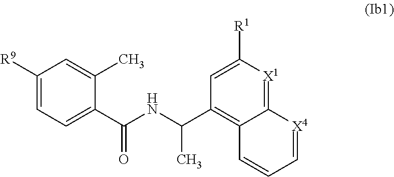

or a pharmaceutically acceptable salt thereof, wherein X¹ is CH or N and X⁴ is CH or X¹ is CH and X⁴ is N.

E20 A compound of embodiment E19 which is of formula:

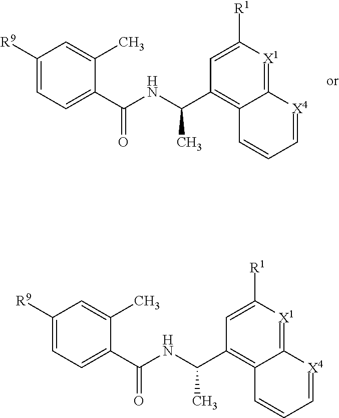

or a pharmaceutically acceptable salt thereof.

E21 A compound of any one of embodiments E1 to E3 having Formula (Ib2):

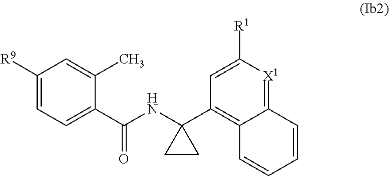

or a pharmaceutically acceptable salt thereof, wherein X¹ is CH or N.

E22 A compound of any one of embodiments E1 to E3 having Formula (Ib3):

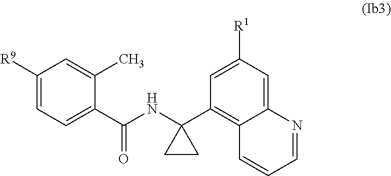

or a pharmaceutically acceptable salt thereof, wherein R⁹ is —(CR^{g}R^{h})—O—(CR^{g}R^{h})R¹².

E23 A compound of any one of embodiments E19 to E21, or a pharmaceutically acceptable salt thereof, wherein:
R⁹ is selected from the group consisting of C₁-C₆ alkyl-C(═O)—OH, —C(═O)—N(R¹¹)R¹², —C(═O)—N(R¹¹)—C₁-C₃ alkyl-R¹², —C(═O)—N(R¹)—(CR^{g}R^{h})—R¹², —(CR^{g}R^{h})—CH(OH)—(CR^{g}R^{h})—R¹², —(CR^{g}R^{h})—CH(OCH₃)—(CR^{g}R^{h})—R¹², —(CR^{g}R^{h})—N(R¹¹)—(CR^{g}R^{h})—R¹², —(CR^{g}R^{h})—O—(CR^{g}R^{h})—R¹², —(CR^{g}R^{h})—S—(CR^{g}R^{h})—R¹², —(CR^{g}R^{h})—S(═O)—(CR^{g}R^{h})—R¹², —C₁-C₃ alkyl-C(═O)—N(R¹¹)R¹², —(CR^{g}R^{h})—C(═O)—N(R¹¹)R¹², —C₁-C₃ alkyl-N(R¹¹)R¹², —C₁-C₃ alkyl-N(R¹¹)—C₁-C₃ alkyl-R¹², —(CR^{g}R^{h})—N(R¹¹)—C(═O)—R¹² and —(CR^{g}R^{h})—OR¹²;

Each of R^{g} and R^{h} is independently selected from the group consisting of H, C₁-C₆ alkyl, C₁-C₆ fluoroalkyl, —C₁-C₆ alkoxy-C₁-C₆ alkyl, or alternatively, R^{g} and R^{h} together with the carbon atom to which they are attached form a C₃-C₆ cycloalkyl;

Each R¹¹ is independently selected from H, —SO₂CH₃, —C(═O)CH₃ and C₁-C₆ alkyl;

Each R¹² is independently selected from C₁-C₆ alkyl, C₃-C₈ cycloalkyl, 4-8 membered heterocycloalkyl and 5-6 membered heteroaryl, wherein said C₃-C₈ cycloalkyl, 4-8 membered heterocycloalkyl and 5-9 membered heteroaryl are each optionally substituted by one or two R¹⁷; and

Each R¹⁷ is independently selected from the group consisting of halogen, C₁-C₆ alkyl, C₁-C₆ hydroxyalkyl, C₁-C₆ fluoroalkyl and C₃-C₆ cycloalkyl.

E24 A compound of embodiment E23, or a pharmaceutically acceptable salt thereof, wherein R⁹ is —CH₂—C(═O)OH, —CH₂NH₂, —CH₂—C(═O)—NH(CH₃), —CH₂—C(═O)—N(CH₃)₂, —CH₂—NH—CH₂-heteroaryl or —CH₂—NH—C(═O)-heteroaryl, —C(═O)—NH—CH₂-heteroaryl, —C(═O)—NH—CH(CH₃)-heteroaryl, —C(═O)—NH—C(CH₃)₂-heteroaryl, —CH₂—C(═O)—NH-heteroaryl, —C(═O)—NH—C(cyclopropyl)-heteroaryl, —C(CH₃)₂—NH—CH₂-heteroaryl, —CH₂—N(CH₃)—CH₂-heteroaryl, —CH₂—N(CH₃)—C(═O)-heteroaryl, —CH(CH₃—)—NH—C(═O)-heteroaryl, —C(cyclopropyl)-NH—C(═O)-heteroaryl, —C(═O)—N(CH₃)—CH₂-heteroaryl, —CH₂—N(SO₂CH₃)—CH₂-heteroaryl, —CH₂—N(C(O)CH₃)—CH₂-heteroaryl, —C(CH₃)₂—NH—C(═O)-heteroaryl, —CH₂—O—CH₂-heteroaryl, —CH₂—S—CH₂-heteroaryl, —CH₂—S(O)—CH₂-heteroaryl, —CH(CH₃)—O—CH₂-heteroaryl, —CH(OH)—C(═O)—NH-heteroaryl, —CH(CF₃)—NH—CH₂-heteroaryl, —CH(CH₂OCH₃)—O—CH₂-heteroaryl, —CH(CH₂OCH₃)—NH—CH₂-heteroaryl, —CH₂—CH(OH)— CH₂-heteroaryl, —CH₂—CH(OCH₃)—CH₂-heteroaryl, —CH₂—O— heteroaryl, wherein said heteroaryl is a pyridinyl or a 5-membered heteroaryl comprising 1 N atom and additionally comprising 1, 2, or 3 heteroatoms selected from N, O and S, said heteroaryl being optionally substituted by one or two R¹⁷ groups selected from methyl, ethyl, isopropyl, hydroxymethyl, chloro, difluoromethyl, trifluoromethyl, cyclopropyl, cyclobutyl or cyclopentyl.

E25 A compound of any one of embodiments E19 to E24, or a pharmaceutically acceptable salt thereof, wherein R⁹ is —CH₂—O—CH₂-heteroaryl and said heteroaryl is a pyridinyl or a 5-membered heteroaryl comprising 1 N atom and additionally comprising 1, 2, or 3 heteroatoms selected from N, O and S.

E26 A compound of embodiment E21, or a pharmaceutically acceptable salt thereof, wherein X¹ is N; R⁹ is —CH₂—O—CH₂-heteroaryl wherein said heteroaryl is pyridinyl, thiazolyl or oxazolyl; and R¹ is a pyrazolyl substituted by —C(═O)—N(CH₃)₂ or —CH₂—C(═O)—N(CH₃)₂.

E27 A compound of embodiment E22, or a pharmaceutically acceptable salt thereof, wherein R⁹ is —CH₂—O—CH₂-heteroaryl wherein said heteroaryl is pyridinyl, thiazolyl or oxazolyl; and R¹ is a pyrazolyl substituted by —C(═O)—N(CH₃)₂ or —CH₂—C(═O)—N(CH₃)₂.

E28 A compound of any one of embodiments E26 or E27, or a pharmaceutically acceptable salt thereof, wherein R⁹ is selected from:

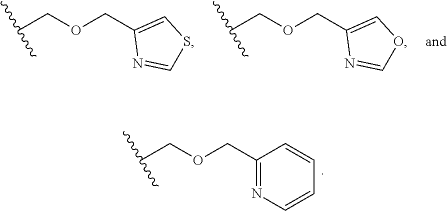

E29 A compound of any one of embodiments E26 to E29, or a pharmaceutically acceptable salt thereof, wherein R¹ is:

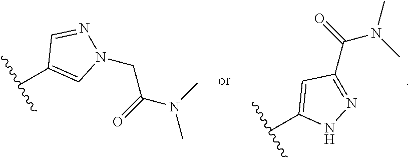 [SMILES: *c1cnn(CC(=O)N(C)C)c1 ; *c1cc(C(=O)N(C)C)n[nH]1]

E30 A compound of any one of embodiments E1 to E2 having the formula (Ic1):

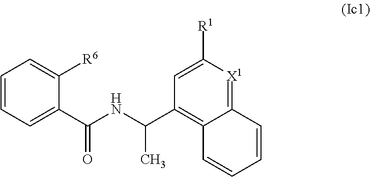

or a pharmaceutically acceptable salt thereof, wherein X¹ is CH or N.

E31 A compound of embodiment E30 which is of formula:

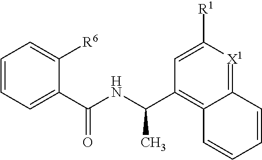

or a pharmaceutically acceptable salt thereof.

E32 A compound of any one of embodiments E1 to E2 having Formula (Ic2):

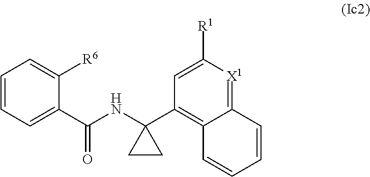

or a pharmaceutically acceptable salt thereof, wherein X¹ is CH or N.

E33 A compound of any one of embodiments E30 to E32, or a pharmaceutically acceptable salt thereof, wherein R⁶ is C₁-C₆ alkyl, C₁-C₃ alkyl-C(═O)—N(R^{e})—N(R)—C(═O)—CH═CH—C(═O)—O—C₁-C₆ alkyl, C₁-C₃ alkyl-C(═O)—N(R^{e})—N(R^{f})—C(═O)—CH═CH—C(═O)—N(R¹¹)R¹², C₁-C₃ alkyl-C(═O)—N(R^{e})—N(R^{f})—C(═O)—CH═CH—O—C₁-C₆ alkyl, C₁-C₃ alkyl-C(═O)—N(R^{e})—N(R)—C(═O)-oxiran-C(═O)—O—C₁-C₆ alkyl or C₁-C₃ alkyl-C(═O)—N(R^{e})—N(R^{f})—C(═O)-oxiran-C(═O)—OH; R^{e} and R^{f} are each independently selected from the group consisting of H and C₁-C₄ alkyl; and R¹¹ and R¹² are each independently selected from the group consisting of H and C₁-C₄ alkyl.

E34 A compound of embodiment E33, or a pharmaceutically acceptable salt thereof, wherein R⁶ is methyl, —C₂H₄—C(═O)—NH—NH—C(═O)—CH═CH—C(═O)—O—CH₃, —C₂H₄—C(═O)—NH—NH—C(═O)—CH═CH—C(═O)—O—C₂H₅, —C₂H₄—C(═O)—NH—NH—C(═O)—CH═CH—C(═O)—O—CH(CH₃)₂, C₂H₄—C(═O)—NH—NH—C(═O)-oxiran-C(═O)—O—C₂H₅, C₂H₄—C(═O)—NH—NH—C(═O)-oxiran-C(═O)—O—CH₃, C₂H₄—C(═O)—NH—NH—C(═O)-oxiran-C(═O)—OH, —C₂H₄—C(═O)—NH—NH—C(═O)—CH═CH—C(═O)—NH₂, —C₂H₄—C(═O)—NH—NH—C(═O)—CH═CH—C(═O)—NH(CH₃), —C₂H₄—C(═O)—NH—NH—C(═O)—CH═CH—C(═O)—N(CH₃)₂ or —C₂H₄—C(═O)—NH—NH—C(═O)—CH═CHO—CH₃.

E35 A compound of any one of embodiments E1 to E25 and E30 to E34, or a pharmaceutically acceptable salt thereof, wherein:
R¹ is selected from the group consisting of -L¹-L²-L³-Cyc¹, C₁-C₆ alkyl, C₁-C₆ aminoalkyl, C₁-C₆ cyanoalkyl, —N(R^{a})R^{b}, —N(R^{a})(C₁-C₆ alkoxy-C₁-C₆ alkyl), —N(R^{a})(C(═O)—C₁-C₆ alkyl), —N(R^{a})(C₁-C₆ alkyl-C(═O)—N(R^{c})R^{d}), —N(R^{a})(C₁-C₆ alkyl-SO₂—N(R^{c})R^{d}), —O—C₁-C₆ hydroxyalkyl and —O—(C₁-C₆ alkoxy-C₁-C₆ alkyl);
R^{a}, R^{b}, R^{c} and R^{d} are each independently selected at each occurrence from the group consisting of H, methyl and ethyl;
L¹, L² and L³ are each either absent or independently selected from the group consisting of O, NH, C(═O), methylene and ethylene;
Cyc¹ is selected from the group consisting of 5-10 membered heteroaryl, C₃-C₈ cycloalkyl and 3-10 membered heterocycloalkyl, each optionally substituted by one, two or three R¹⁰;

Each R¹⁰ is independently selected at each occurrence from the group consisting of cyano, halogen, oxo, amino, C₁-C₆ alkyl, C₁-C₆ fluoroalkyl, —C₁-C₆ alkoxy-C₁-C₆ alkyl, —COOH, —C(═O)—O—C₁-C₆ alkyl, —C(═O)—N(R¹¹)R¹², —S(O)₂R¹², —S(O)₂N(R¹¹)R¹², —NH—S(O)₂N(R¹¹)R¹², NH—S(O)₂R¹²—OR¹²; and

Each R¹¹ and R¹² is independently H, methyl or ethyl.

E36 A compound of embodiment E35, or a pharmaceutically acceptable salt thereof, wherein R¹ is selected from the group consisting of 5-9 membered heteroaryl optionally substituted by one, two or three R¹⁰ selected from the group consisting of C₁-C₄ alkyl, cyano, fluoro, oxo, amino, —CF₃, —CHF₂, —CH₂CHF₂, —CH₂—O—CH₃, —C₂H₄O—CH₃, —COOH, —C(═O)—O—CH₃, —C(═O)—O—C₂H₅, —C(═O)—NH₂, —C(═O)—NH(CH₃), —C(═O)—N(CH₃)₂, —S(O)₂—CH₃, —S(O)₂—C₂H₅, —S(O)₂N(CH₃)₂, —NH—S(O)₂—CH₃, —OCH₃ and cyclopropyl; 5-membered heterocycloalkyl optionally substituted by oxo; C₄ cycloalkyl substituted by two fluroro; L¹-L²-L³-Cyc¹ wherein -L¹-L²-L³ is —NH, —O, —OCH₂, —CH₂, —C₂H₄, —NHCH₂ or —NHCH₂C(═O) and Cyc¹ is a 4-6 membered heterocycloalkyl or a 5-membered heteroaryl, each optionally substituted by one or two groups selected from oxo and methyl; propyl; —CH₂NH₂; —CH₂CN; amino; —NH(CH₃); —N(CH₃)₂; —NHCOCH₃; —NH—C₂H₄—SO₂—N(CH₃)₂; —N(C₂H₅)(C₂H₄—O—CH₃); —N(CH₃)(C₂H₄—C(═O)—N(CH₃)₂); —O—C₂H₄—OH; and —O—C₂H₄—O—CH₃.

E37 A compound of embodiment E36, or a pharmaceutically acceptable salt thereof, wherein R¹ is:

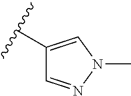 [SMILES: *c1cnn(C)c1]

E38 A compound of embodiment E1 having Formula (Idi):

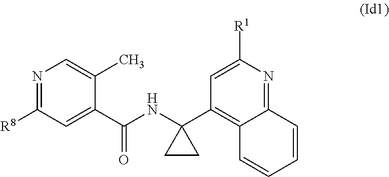

or a pharmaceutically acceptable salt thereof.

E39 A compound of embodiment E38, or a pharmaceutically acceptable salt thereof, wherein R¹ is a 5-10 membered heteroaryl which is optionally substituted by one C₁-C₆ alkyl and R⁸ is a 4-10 membered heterocycloalkyl which is optionally substituted by one C₁-C₆ alkyl.

E40 A compound of embodiment E39, or a pharmaceutically acceptable salt thereof, wherein R¹ is:

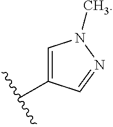 [SMILES: *c1cnn(C)c1]

E41 A compound of embodiment E1 which is selected from the group consisting of:
N1-(1-(3-(1H-1,2,3-triazol-1-yl)naphthalen-1-yl)ethyl)-6-methylisophthalamide;
N1-(1-(3-(2H-1,2,3-triazol-2-yl)naphthalen-1-yl)ethyl)-6-methylisophthalamide;
5-(aminomethyl)-2-methyl-N-(1-(3-(1-methyl-1H-pyrazol-4-yl)naphthalen-1-yl)ethyl)benzamide;
(2R)—N-(4-methyl-3-{[(1RS)-1-[3-(1-methyl-1H-pyrazol-4-yl)naphthalen-1-yl]ethyl]carbamoyl}phenyl)piperidine-2-carboxamide;
4-(aminomethyl)-2-methyl-N-(1-(3-(1-methyl-1H-pyrazol-4-yl)naphthalen-1-yl)ethyl)benzamide;
4-((((1H-imidazol-2-yl)methyl)amino)methyl)-2-methyl-N-(1-(3-(1-methyl-1H-pyrazol-4-yl)naphthalen-1-yl)ethyl)benzamide;
2-methyl-N-(1-(3-(1-methyl-1H-pyrazol-4-yl)naphthalen-1-yl)ethyl)-4-(((thiazol-4-ylmethyl)amino)methyl)benzamide;
rel-(R)-5-(aminomethyl)-2-methyl-N-(1-(3-(1-methyl-1H-pyrazol-4-yl)naphthalen-1-yl)ethyl)benzamide, ENT-1;
rel-(R)-5-(aminomethyl)-2-methyl-N-(1-(3-(1-methyl-1H-pyrazol-4-yl)naphthalen-1-yl)ethyl)benzamide, ENT-2;
2-methyl-N-(1-(3-(1-methyl-1H-pyrazol-4-yl)naphthalen-1-yl)ethyl)-5-((methylamino)methyl)benzamide;
5-(aminomethyl)-2-methyl-N-[(1R)-1-[2-(1-methyl-1H-pyrazol-4-yl)quinolin-4-yl]ethyl]benzamide;
2-methyl-N-[(1R)-1-[2-(1-methyl-1H-pyrazol-3-yl)quinolin-4-yl]ethyl]benzamide;
2-methyl-N-[(1R)-1-[2-(1-methyl-1H-pyrazol-4-yl)quinolin-4-yl]ethyl]-5-[(methylamino)methyl]benzamide;
(2R)—N-(4-methyl-3-{[(1R)-1-[2-(1-methyl-1H-pyrazol-4-yl)quinolin-4-yl]ethyl]carbamoyl}phenyl)piperidine-2-carboxamide;
(2R)—N-methyl-N-(4-methyl-3-{[(1R)-1-[2-(1-methyl-1H-pyrazol-3-yl)quinolin-4-yl]ethyl]carbamoyl}phenyl)piperidine-2-carboxamide;
5-(aminomethyl)-2-methyl-N-[(1R)-1-[2-(4H-1,2,4-triazol-4-yl)quinolin-4-yl]ethyl]benzamide;
2-methyl-5-[(methylamino)methyl]-N-[(1R)-1-[2-(1H-pyrazol-4-yl)quinolin-4-yl]ethyl]benzamide;
5-(aminomethyl)-2-methyl-N-[(1R)-1-[2-(1H-pyrazol-4-yl)quinolin-4-yl]ethyl]benzamide; methyl 5-{4-[(1R)-1-{[5-(aminomethyl)-2-methylphenyl]formamido}ethyl]quinolin-2-yl}-1H-pyrrole-3-carboxylate;
5-{4-[(1R)-1-{[5-(aminomethyl)-2-methylphenyl]formamido}ethyl]quinolin-2-yl}-1H-pyrrole-3-carboxylic acid;
5-{4-[(1R)-1-{[5-(aminomethyl)-2-methylphenyl]formamido}ethyl]quinolin-2-yl}-1H-pyrrole-3-carboxamide;
5-{4-[(1R)-1-{[5-(aminomethyl)-2-methylphenyl]formamido}ethyl]quinolin-2-yl}-N-methyl-1H-pyrrole-3-carboxamide;
5-(aminomethyl)-2-methyl-N-[(1R)-1-[2-(1H-1,2,3-triazol-1-yl)quinolin-4-yl]ethyl]benzamide;
5-(aminomethyl)-2-methyl-N-[(1R)-1-[2-(1H-1,2,4-triazol-1-yl)quinolin-4-yl]ethyl]benzamide;
(2R)—N-(3-{[(1RS)-1-(2-aminoquinolin-4-yl)ethyl]carbamoyl}-4-methylphenyl)piperidine-2-carboxamide;
(R)—N-(3-(((R*)-1-(2-aminoquinolin-4-yl)ethyl)carbamoyl)-4-methylphenyl)piperidine-2-carboxamide, ENT-1;
(R)—N-(3-(((R*)-1-(2-aminoquinolin-4-yl)ethyl)carbamoyl)-4-methylphenyl)piperidine-2-carboxamide, ENT-2;
(R)—N-(3-(((R*)-1-(2-(1H-1,2,3-triazol-1-yl)quinolin-4-yl)ethyl)carbamoyl)-4-methylphenyl)piperidine-2-carboxamide, ENT-1;
(R)—N-(3-(((R*)-1-(2-(1H-1,2,3-triazol-1-yl)quinolin-4-yl)ethyl)carbamoyl)-4-methylphenyl)piperidine-2-carboxamide, ENT-2;
5-(aminomethyl)-2-methyl-N-(1-(2-(4-(trifluoromethyl)-1H-1,2,3-triazol-1-yl)quinolin-4-yl)ethyl)benzamide;
5-(aminomethyl)-2-methyl-N-(1-(2-(4-(trifluoromethyl)-2H-1,2,3-triazol-2-yl)quinolin-4-yl)ethyl)benzamide;
4-(4-(1-(5-(aminomethyl)-2-methylbenzamido)ethyl)quinolin-2-yl)-4H-1,2,4-triazole-3-carboxamide;
5-(aminomethyl)-2-methyl-N-(1-(2-(2-oxopyrrolidin-1-yl)quinolin-4-yl)ethyl)benzamide;
5-(aminomethyl)-N-(1-(2-(3-cyano-1H-pyrazol-1-yl)quinolin-4-yl)ethyl)-2-methylbenzamide;
N-(1-(2-(1H-imidazol-1-yl)quinolin-4-yl)ethyl)-5-(aminomethyl)-2-methylbenzamide;
2-methyl-N-[(1R)-1-{2-[(oxetan-3-yl)amino]quinolin-4-yl}ethyl]benzamide;
2-methyl-N-[(1R)-1-{2-[(1-methyl-1H-pyrazol-3-yl)amino]quinolin-4-yl}ethyl]benzamide;
N-[(1R)-1-(2-aminoquinolin-4-yl)ethyl]-2-methylbenzamide;
N-[(1R)-1-(2-acetamidoquinolin-4-yl)ethyl]-2-methylbenzamide;
2-methyl-N-[(1R)-1-[2-(methylamino)quinolin-4-yl]ethyl]benzamide;
N-[(1R)-1-[2-(dimethylamino)quinolin-4-yl]ethyl]-2-methylbenzamide;
2-methyl-N-[(1RS)-1-(2-{[(3RS&)-oxolan-3-yl]methoxy}quinolin-4-yl)ethyl]benzamide;
(R)-2-methyl-N-(1-(2-(oxetan-3-ylmethoxy)quinolin-4-yl)ethyl)benzamide;
(R)—N-(1-(2-(2-hydroxyethoxy)quinolin-4-yl)ethyl)-2-methylbenzamide;
2-methyl-N-[(1RS)-1-{2-[(3RS&)-oxolan-3-yloxy]quinolin-4-yl}ethyl]benzamide;
(R)—N-(1-(2-(2-methoxyethoxy)quinolin-4-yl)ethyl)-2-methylbenzamide;
2-methyl-N-[(1R)-1-[2-(1H-pyrazol-5-yl)quinolin-4-yl]ethyl]benzamide;
2-methyl-N-[(1R)-1-[2-(1-methyl-1H-pyrazol-4-yl)quinolin-4-yl]ethyl]benzamide;
2-methyl-N-[(1R)-1-[2-(1-methyl-1H-pyrazol-5-yl)quinolin-4-yl]ethyl]benzamide;
(2R)—N-(4-methyl-3-{[(1RS)-1-[3-(1H-pyrazol-4-yl)naphthalen-1-yl]ethyl]carbamoyl}phenyl)piperidine-2-carboxamide;
(2R)—N-(4-methyl-3-{[(1R*)-1-[3-(1H-pyrazol-4-yl)naphthalen-1-yl]ethyl]carbamoyl}phenyl)piperidine-2-carboxamide;
(2R)—N-(4-methyl-3-{[(1R*)-1-[3-(1H-pyrazol-4-yl)naphthalen-1-yl]ethyl]carbamoyl}phenyl)piperidine-2-carboxamide;
N-(3-methyl-4-((1-(3-(1-methyl-1H-pyrazol-4-yl)naphthalen-1-yl)ethyl)carbamoyl)benzyl)thiazole-4-carboxamide;
2-methyl-N-[(1R)-1-[2-(1-methyl-1H-pyrazol-4-yl)quinolin-4-yl]ethyl]-5-(piperazin-1-yl)benzamide;
5-{4-[(1R)-1-{[5-(aminomethyl)-2-methylphenyl]formamido}ethyl]quinolin-2-yl}-N,N-dimethyl-1H-pyrrole-3-carboxamide;
methyl 5-{4-[(1R)-1-({2-methyl-5-[(methylamino)methyl]phenyl}formamido)ethyl]quinolin-2-yl}-1H-pyrrole-3-carboxylate;
2-methyl-N-[(1R)-1-[2-(1,2-oxazol-4-yl)quinolin-4-yl]ethyl]benzamide;
2-methyl-N-(1-(3-(1-methyl-1H-pyrazol-4-yl)naphthalen-1-yl)ethyl)-5-(piperazin-1-yl)benzamide;
2-methyl-N-[(1R)-1-[2-(1-methyl-1H-pyrazol-4-yl)quinolin-4-yl]ethyl]-5-(4-methylpiperazin-1-yl)benzamide;
5-(methanesulfonamidomethyl)-2-methyl-N-[(1R)-1-[2-(1-methyl-1H-pyrazol-4-yl)quinolin-4-yl]ethyl]benzamide;
N-(3-methyl-4-((1-(3-(1-methyl-1H-pyrazol-4-yl)naphthalen-1-yl)ethyl)carbamoyl)benzyl)-1H-imidazole-2-carboxamide;
methyl(2E)-4-[3-(2-{[(1R)-1-[2-(1-methyl-1H-pyrazol-4-yl)quinolin-4-yl]ethyl]carbamoyl}phenyl)propanehydrazido]-4-oxobut-2-enoate;
methyl (E)-4-(2-(3-(2-((1-(3-(1-methyl-1H-pyrazol-4-yl)naphthalen-1-yl)ethyl)carbamoyl)phenyl)propanoyl)hydrazineyl)-4-oxobut-2-enoate;
(2R)—N-(4-methyl-3-{[(1RS)-1-[3-(1H-pyrazol-5-yl)naphthalen-1-yl]ethyl]carbamoyl}phenyl)piperidine-2-carboxamide;
(R)—N-(4-methyl-3-(((R*)-1-(3-(1-methyl-1H-pyrazol-4-yl)naphthalen-1-yl)ethyl)carbamoyl)phenyl)piperidine-2-carboxamide, ENT-1;
(R)—N-(4-methyl-3-(((R*)-1-(3-(1-methyl-1H-pyrazol-4-yl)naphthalen-1-yl)ethyl)carbamoyl)phenyl)piperidine-2-carboxamide, ENT-2;
(R)—N-(3-(((R*)-1-(3-(1H-pyrazol-5-yl)naphthalen-1-yl)ethyl)carbamoyl)-4-methylphenyl)piperidine-2-carboxamide, ENT-1;
(R)—N-(3-(((R*)-1-(3-(1H-pyrazol-5-yl)naphthalen-1-yl)ethyl)carbamoyl)-4-methylphenyl)piperidine-2-carboxamide, ENT-2;
methyl 5-(4-(1-(5-(aminomethyl)-2-methylbenzamido)ethyl)naphthalen-2-yl)-1H-pyrrole-3-carboxylate;
5-(4-(1-(5-(aminomethyl)-2-methylbenzamido)ethyl)naphthalen-2-yl)-N-methyl-1H-pyrrole-3-carboxamide;
5-(4-(1-(5-(aminomethyl)-2-methylbenzamido)ethyl)naphthalen-2-yl)-N,N-dimethyl-1H-pyrrole-3-carboxamide;
2-methyl-N-(1-(3-(1-methyl-1H-pyrazol-4-yl)naphthalen-1-yl)ethyl)-5-(4-methylpiperazin-1-yl)benzamide;
2-methyl-N-{1-[2-(1H-pyrazol-4-yl)quinolin-4-yl]ethyl}benzamide;
2-methyl-N-{1-[2-(4-methyl-1H-pyrazol-5-yl)quinolin-4-yl]ethyl}benzamide;
N-{1-[2-(5-cyanothiophen-3-yl)quinolin-4-yl]ethyl}-2-methylbenzamide;
2-methyl-N-[1-(2-{2H,3H-thieno[3,4-b][1,4]dioxin-5-yl}quinolin-4-yl)ethyl]benzamide;
N-(1-{2-[1-(difluoromethyl)-1H-pyrazol-4-yl]quinolin-4-yl}ethyl)-2-methylbenzamide;
N-(1-{2-[5-(methoxymethyl)thiophen-3-yl]quinolin-4-yl}ethyl)-2-methylbenzamide;
N-{1-[2-(3-cyclopropyl-1-methyl-1H-pyrazol-5-yl)quinolin-4-yl]ethyl}-2-methylbenzamide;
N-{1-[2-(4-cyano-1-methyl-1H-pyrazol-5-yl)quinolin-4-yl]ethyl}-2-methylbenzamide;
N-{1-[2-(2-methoxypyridin-4-yl)quinolin-4-yl]ethyl}-2-methylbenzamide;
N-{1-[2-(1-ethyl-1H-pyrazol-4-yl)quinolin-4-yl]ethyl}-2-methylbenzamide;
N-{1-[2-(2,1,3-benzoxadiazol-5-yl)quinolin-4-yl]ethyl}-2-methylbenzamide;
N-(1-{2-[1-(2-methoxyethyl)-1H-pyrazol-4-yl]quinolin-4-yl}ethyl)-2-methylbenzamide;
N-{1-[2-(1-tert-butyl-1H-pyrazol-4-yl)quinolin-4-yl]ethyl}-2-methylbenzamide;
2-methyl-N-{1-[2-(pyridin-3-yl)quinolin-4-yl]ethyl}benzamide;
N-{1-[2-(1-ethyl-3-methyl-1H-pyrazol-4-yl)quinolin-4-yl]ethyl}-2-methylbenzamide;
2-methyl-N-{1-[2-(2-oxo-2,3-dihydro-1,3-benzoxazol-5-yl)quinolin-4-yl]ethyl}benzamide;
2-methyl-N-{1-[2-(5-methyl-1H-pyrrol-3-yl)quinolin-4-yl]ethyl}benzamide;
2-methyl-N-[1-(2-{[1,2,4]triazolo[1,5-a]pyridin-6-yl}quinolin-4-yl)ethyl]benzamide;
2-methyl-N-{1-[2-(thiophen-3-yl)quinolin-4-yl]ethyl}benzamide;
N-(1-{2-[1-(2,2-difluoroethyl)-1H-pyrazol-4-yl]quinolin-4-yl}ethyl)-2-methylbenzamide;
N-(1-{2-[1-(2-methoxyethyl)-3,5-dimethyl-1H-pyrazol-4-yl]quinolin-4-yl}ethyl)-2-methylbenzamide;
N-{1-[2-(4-methanesulfonamidophenyl)quinolin-4-yl]ethyl}-2-methylbenzamide;
N-[1-(2-{1-[(1-cyanocyclopropyl)methyl]-1H-pyrazol-4-yl}quinolin-4-yl)ethyl]-2-methylbenzamide;
2-methyl-N-(1-{2-[3-(propan-2-yl)-1,2-oxazol-5-yl]quinolin-4-yl}ethyl)benzamide;
2-methyl-N-[1-(2-{1-[2-(morpholin-4-yl)ethyl]-1H-pyrazol-4-yl}quinolin-4-yl)ethyl]benzamide;
N-{1-[2-(1,3-dimethyl-1H-pyrazol-4-yl)quinolin-4-yl]ethyl}-2-methylbenzamide;
N-{1-[2-(6-aminopyridin-3-yl)quinolin-4-yl]ethyl}-2-methylbenzamide;
methyl 5-(4-{1-[(2-methylphenyl)formamido]ethyl}quinolin-2-yl)-1H-pyrrole-3-carboxylate;
N-{1-[2-(1,5-dimethyl-1H-pyrazol-4-yl)quinolin-4-yl]ethyl}-2-methylbenzamide;
2-methyl-N-{1-[2-(pyrimidin-5-yl)quinolin-4-yl]ethyl}benzamide;
2-methyl-N-[1-(2-{pyrazolo[1,5-a]pyrimidin-3-yl}quinolin-4-yl)ethyl]benzamide;
2-methyl-N-(1-{2-[1-methyl-3-(trifluoromethyl)-1H-pyrazol-4-yl]quinolin-4-yl}ethyl)benzamide;
methyl 5-(4-{1-[(2-methylphenyl)formamido]ethyl}quinolin-2-yl)furan-2-carboxylate;
2-methyl-N-{1-[2-(1-methyl-1H-1,2,3-benzotriazol-5-yl)quinolin-4-yl]ethyl}benzamide;
2-methyl-N-{1-[2-(2-methyl-2H-1,2,3-triazol-4-yl)quinolin-4-yl]ethyl}benzamide;
2-methyl-N-(1-{2-[3-methyl-1-(propan-2-yl)-1H-pyrazol-4-yl]quinolin-4-yl}ethyl)benzamide;
N-{1-[2-(5-fluoropyridin-3-yl)quinolin-4-yl]ethyl}-2-methylbenzamide;
2-methyl-N-[1-(2-{[(1,3-oxazol-4-yl)methyl]amino}quinolin-4-yl)ethyl]benzamide;
2-methyl-N-[1-(2-{[(1,3-oxazol-5-yl)methyl]amino}quinolin-4-yl)ethyl]benzamide;
2-methyl-N-[1-(2-{[(oxan-3-yl)methyl]amino}quinolin-4-yl)ethyl]benzamide
2-methyl-N-[1-(2-{[(oxan-4-yl)methyl]amino}quinolin-4-yl)ethyl]benzamide;
2-methyl-N-(1-{2-[(oxolan-3-yl)amino]quinolin-4-yl}ethyl)benzamide;
N-[1-(2-{[2-(dimethylcarbamoyl)ethyl](methyl)amino}quinolin-4-yl)ethyl]-2-methylbenzamide;
N-[1-(2-{[(1,1-dioxo-1λ⁶-thian-4-yl)methyl]amino}quinolin-4-yl)ethyl]-2-methylbenzamide;
N-(1-{2-[(1-cyanocyclopropyl)amino]quinolin-4-yl}ethyl)-2-methylbenzamide;
2-methyl-N-[1-(2-{[2-(morpholin-4-yl)-2-oxoethyl]amino}quinolin-4-yl)ethyl]benzamide;
2-methyl-N-(1-{2-[(oxan-3-yl)amino]quinolin-4-yl}ethyl)benzamide
N-[1-(2-{[2-(dimethylsulfamoyl)ethyl]amino}quinolin-4-yl)ethyl]-2-methylbenzamide;
N-(1-{2-[ethyl(2-methoxyethyl)amino]quinolin-4-yl}ethyl)-2-methylbenzamide;
N-[1-(2-{[(1-methanesulfonylpiperidin-4-yl)methyl]amino}quinolin-4-yl)ethyl]-2-methylbenzamide;
N-(1-{2-[4-(dimethylsulfamoyl)piperidin-1-yl]quinolin-4-yl}ethyl)-2-methylbenzamide;
2-methyl-N-[(1R)-1-{2-[(oxan-4-yl)methyl]quinolin-4-yl}ethyl]benzamide;
2-methyl-N-[(1R)-1-(2-propylquinolin-4-yl)ethyl]benzamide;
N-[(1R)-1-[2-(3,3-difluorocyclobutyl)quinolin-4-yl]ethyl]-2-methylbenzamide;
2-methyl-N-[(1R)-1-[2-(oxan-4-yl)quinolin-4-yl]ethyl]benzamide;
2-methyl-N-[(1R)-1-{2-[(2-oxopyrrolidin-1-yl)methyl]quinolin-4-yl}ethyl]benzamide;
2-methyl-N-[(1R)-1-{2-[2-(1H-pyrazol-1-yl)ethyl]quinolin-4-yl}ethyl]benzamide;
N-[(1R)-1-[2-(aminomethyl)quinolin-4-yl]ethyl]-2-methylbenzamide;
N-[(1R)-1-[2-(cyanomethyl)quinolin-4-yl]ethyl]-2-methylbenzamide;
N-[(1R)-1-(2-chloroquinolin-4-yl)ethyl]-2-methylbenzamide;
2-methyl-N-[(1R)-1-[2-(1-methyl-1H-pyrazol-4-yl)quinolin-4-yl]ethyl]-4-({[(1,3-thiazol-4-yl)methyl]amino}methyl)benzamide;
5-(aminomethyl)-2-methyl-N-{1-[3-(1-methyl-1H-pyrazol-4-yl)naphthalen-1-yl]cyclopropyl}benzamide;
(R)-2-(3-methyl-4-((1-(2-(1-methyl-1H-pyrazol-4-yl)quinolin-4-yl)ethyl)carbamoyl)phenyl) acetic acid;
(R)-2-methyl-N1-(1-(2-(1-methyl-1H-pyrazol-4-yl)quinolin-4-yl)ethyl)-N4-(thiazol-4-ylmethyl)terephthalamide;
2-methyl-N1-((R)-1-(2-(i-methyl-1H-pyrazol-4-yl)quinolin-4-yl)ethyl)-N4-(1-(thiazol-4-yl)ethyl)terephthalamide;
(R)-2-methyl-N1-(i-(2-(i-methyl-1H-pyrazol-4-yl)quinolin-4-yl)ethyl)-N4-((5-methylthiazol-4-yl)methyl)terephthalamide
(R)-2-methyl-N1-(i-(2-(i-methyl-1H-pyrazol-4-yl)quinolin-4-yl)ethyl)-N4-((2-methylthiazol-4-yl)methyl)terephthalamide;
5-(aminomethyl)-N-(1-(4-methoxy-3-(1-methyl-1H-pyrazol-4-yl)naphthalen-1-yl)ethyl)-2-methylbenzamide;
2-methyl-N-(1-(2-(1-methyl-1H-pyrazol-4-yl)quinolin-4-yl)cyclopropyl)-5-(piperazin-1-yl)benzamide;
5-(aminomethyl)-2-methyl-N-(1-(2-(1-methyl-1H-pyrazol-4-yl)quinolin-4-yl)cyclopropyl)benzamide;
5-(hexahydropyrrolo[3,4-c]pyrrol-2(1H)-yl)-2-methyl-N-(1-(2-(1-methyl-1H-pyrazol-4-yl)quinolin-4-yl)cyclopropyl)benzamide;
5-(2,5-diazabicyclo[2.2.1]heptan-2-yl)-2-methyl-N-(1-(2-(1-methyl-1H-pyrazol-4-yl)quinolin-4-yl)cyclopropyl)benzamide;
2-methyl-N-(1-(2-(1-methyl-1H-pyrazol-4-yl)quinolin-4-yl)cyclopropyl)-4-(((thiazol-4-ylmethyl)amino)methyl)benzamide;
2-methyl-N-(1-(2-(1-methyl-1H-pyrazol-4-yl)quinolin-4-yl)cyclopropyl)-5-(4-methylpiperazin-1-yl)benzamide;
(R)—N-(1-(3-methyl-4-((1-(2-(1-methyl-1H-pyrazol-4-yl)quinolin-4-yl)ethyl) carbamoyl)phenyl)cyclopropyl)thiazole-4-carboxamide;
(R)—N-(1-(3-methyl-4-((1-(2-(1-methyl-1H-pyrazol-4-yl)quinolin-4-yl)ethyl)carbamoyl)phenyl)cyclopropyl)-1H-imidazole-2-carboxamide;
(R)—N-(2-(3-methyl-4-((1-(2-(1-methyl-1H-pyrazol-4-yl)quinolin-4-yl)ethyl) carbamoyl)phenyl)propan-2-yl)thiazole-4-carboxamide;
(R)—N-(2-(3-methyl-4-((1-(2-(1-methyl-1H-pyrazol-4-yl)quinolin-4-yl)ethyl) carbamoyl)phenyl)propan-2-yl)-1H-imidazole-2-carboxamide;
5-(4-(1-(2-methyl-5-(4-methylpiperazin-1-yl)benzamido)ethyl)naphthalen-2-yl)-1H-pyrrole-3-carboxylic acid;
5-(aminomethyl)-N-(1-(4-hydroxy-3-(1-methyl-1H-pyrazol-4-yl)naphthalen-1-yl)ethyl)-2-methylbenzamide;
2-methyl-N-(1-(2-(1-methyl-1H-pyrazol-4-yl)quinolin-4-yl)cyclopropyl)-5-(5-methylhexahydropyrrolo[3,4-c]pyrrol-2(1H)-yl)benzamide;
(2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylic acid;
methyl(2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylate;
(R)-2-methyl-4-((((1-methyl-1H-imidazol-5-yl)methyl)amino)methyl)-N-(1-(2-(1-methyl-1H-pyrazol-4-yl)quinolin-4-yl)ethyl)benzamide;
(R,E)-2-(3-(2-(3-methoxyacryloyl) hydrazineyl)-3-oxopropyl)-N-(1-(2-(1-methyl-1H-pyrazol-4-yl)quinolin-4-yl)ethyl)benzamide;
(R,E)-N-(1-(2-(1-methyl-1H-pyrazol-4-yl)quinolin-4-yl)ethyl)-2-(3-(2-(4-(methylamino)-4-oxobut-2-enoyl)hydrazineyl)-3-oxopropyl)benzamide;
(R,E)-2-(3-(2-(4-amino-4-oxobut-2-enoyl)hydrazineyl)-3-oxopropyl)-N-(1-(2-(1-methyl-1H-pyrazol-4-yl)quinolin-4-yl)ethyl)benzamide;
methyl (E)-4-(2-(3-(2-((1-(2-(1-methyl-1H-pyrazol-4-yl)quinolin-4-yl)cyclopropyl) carbamoyl)phenyl) propanoyl)hydrazineyl)-4-oxobut-2-enoate;
(E)-2-(3-(2-(4-amino-4-oxobut-2-enoyl)hydrazineyl)-3-oxopropyl)-N-(1-(2-(1-methyl-1H-pyrazol-4-yl)quinolin-4-yl)cyclopropyl)benzamide;
ethyl(2S,3S)-3-(2-(3-(2-(((R)-1-(2-(1-methyl-1H-pyrazol-4-yl)quinolin-4-yl)ethyl)carbamoyl)phenyl) propanoyl)hydrazine-1-carbonyl)oxirane-2-carboxylate;
methyl 5-(4-(1-(2-methyl-5-(4-methylpiperazin-1-yl)benzamido)ethyl) naphthalen-2-yl)-1H-pyrrole-3-carboxylate;
N,N-dimethyl-5-(4-(1-(2-methyl-5-(4-methylpiperazin-1-yl)benzamido)ethyl) naphthalen-2-yl)-1H-pyrrole-3-carboxamide;
2-methyl-N-(1-(3-(1-methyl-1H-pyrazol-4-yl)naphthalen-1-yl)cyclopropyl)-5-(piperazin-1-yl)benzamide;
2-methyl-N-(1-(3-(1-methyl-1H-pyrazol-4-yl)naphthalen-1-yl)cyclopropyl)-5-(4-methylpiperazin-1-yl)benzamide;
ethyl(R,E)-4-(2-(3-(2-((1-(2-(1-methyl-1H-pyrazol-4-yl)quinolin-4-yl)ethyl) carbamoyl)phenyl) propanoyl)hydrazineyl)-4-oxobut-2-enoate;
isopropyl(R,E)-4-(2-(3-(2-((1-(2-(1-methyl-1H-pyrazol-4-yl)quinolin-4-yl)ethyl)carbamoyl)phenyl) propanoyl)hydrazineyl)-4-oxobut-2-enoate;
(R,E)-2-(3-(2-(4-(dimethylamino)-4-oxobut-2-enoyl)hydrazineyl)-3-oxopropyl)-N-(1-(2-(1-methyl-1H-pyrazol-4-yl)quinolin-4-yl)ethyl)benzamide;
5-(3-amino-2-oxopiperidin-1-yl)-2-methyl-N—((R)-1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-(2-oxo-2-(thiazol-4-ylamino)ethyl)benzamide;
(R)-2-methyl-N-(1-(2-(1-methyl-1H-pyrazol-4-yl)quinolin-4-yl)ethyl)-4-(2-oxo-2-(thiazol-5-ylamino)ethyl)benzamide;
(R)-2-methyl-N-(1-(2-(1-methyl-1H-pyrazol-4-yl)quinolin-4-yl)ethyl)-4-(2-oxo-2-(thiazol-2-ylamino)ethyl)benzamide;
(R)-4-(2-((1H-imidazol-2-yl)amino)-2-oxoethyl)-2-methyl-N-(1-(2-(1-methyl-1H-pyrazol-4-yl)quinolin-4-yl)ethyl)benzamide;
2-methyl-N—((R)-1-(2-(1-methyl-1H-pyrazol-4-yl)quinolin-4-yl)ethyl)-5-(1-oxo-2,6-diazaspiro[4.5]decan-2-yl)benzamide;
(R)-2-methyl-N-(1-(2-(1-methyl-1H-pyrazol-4-yl)quinolin-4-yl)ethyl)-4-(2-((thiazol-4-ylmethyl)amino)propan-2-yl)benzamide;
(R)—N4-(1-(1H-imidazol-2-yl)cyclopropyl)-2-methyl-N1-(1-(2-(1-methyl-1H-pyrazol-4-yl)quinolin-4-yl)ethyl)terephthalamide;
(R)—N⁴,2-dimethyl-N¹-(1-(2-(1-methyl-1H-pyrazol-4-yl)quinolin-4-yl)ethyl)-N4-(thiazol-4-ylmethyl)terephthalamide;
(R)-N4-(2-(1H-imidazol-2-yl)propan-2-yl)-2-methyl-N1-(1-(2-(1-methyl-1H-pyrazol-4-yl)quinolin-4-yl)ethyl)terephthalamide;
(R)-N4-((1H-imidazol-2-yl)methyl)-2-methyl-N1-(1-(2-(1-methyl-1H-pyrazol-4-yl)quinolin-4-yl)ethyl)terephthalamide;
N4-(1-(1H-imidazol-2-yl)ethyl)-2-methyl-N1-((R)-1-(2-(1-methyl-1H-pyrazol-4-yl)quinolin-4-yl)ethyl)terephthalamide;
5-(3-amino-2-oxopyrrolidin-1-yl)-2-methyl-N—((R)-1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-5-(2-oxopiperazin-1-yl)benzamide;
(R)-2-methyl-N-(1-(2-(1-methyl-1H-pyrazol-4-yl)quinolin-4-yl)ethyl)-5-(4-methyl-2-oxopiperazin-1-yl)benzamide;
ethyl(2R,3R)-3-(2-(3-(2-(((R)-1-(2-(1-methyl-1H-pyrazol-4-yl)quinolin-4-yl)ethyl)carbamoyl)phenyl) propanoyl)hydrazine-1-carbonyl)oxirane-2-carboxylate;
5-(3,6-diazabicyclo[3.1.1]heptan-3-yl)-2-methyl-N—((R)-1-(2-(1-methyl-1H-pyrazol-4-yl)quinolin-4-yl)ethyl)benzamide;
2-methyl-N—((R)-1-(2-(1-methyl-1H-pyrazol-4-yl)quinolin-4-yl)ethyl)-5-(6-methyl-3,6-diazabicyclo[3.1.1]heptan-3-yl)benzamide;
2-methyl-N4-((1-methyl-1H-imidazol-2-yl)methyl)-N¹-(1-(2-(1-methyl-1H-pyrazol-4-yl)quinolin-4-yl)cyclopropyl) terephthalamide;
2-methyl-N¹-(1-(2-(1-methyl-1H-pyrazol-4-yl)quinolin-4-yl)cyclopropyl)-N4-(thiazol-4-ylmethyl)terephthalamide;
5-(2,5-diazabicyclo[2.2.1]heptan-2-yl)-2-methyl-N—((R)-1-(2-(1-methyl-1H-pyrazol-4-yl)quinolin-4-yl)ethyl)benzamide;
2-methyl-N—((R)-1-(2-(1-methyl-1H-pyrazol-4-yl)quinolin-4-yl)ethyl)-5-(5-methyl-2,5-diazabicyclo[2.2.1]heptan-2-yl)benzamide;
(R)-4-(4-(1-(5-(aminomethyl)-2-methylbenzamido)ethyl) quinolin-2-yl)-N,1-dimethyl-1H-pyrrole-2-carboxamide;
(R)-4-(4-(1-(5-(aminomethyl)-2-methylbenzamido)ethyl) quinolin-2-yl)-N,N,1-trimethyl-1H-pyrrole-2-carboxamide;
(R)-2-methyl-N-(1-(2-(1-methyl-1H-pyrazol-4-yl)quinolin-4-yl)ethyl)-4-(2-(methylamino)-2-oxoethyl)benzamide;
(R)-4-(2-(dimethylamino)-2-oxoethyl)-2-methyl-N-(1-(2-(1-methyl-1H-pyrazol-4-yl)quinolin-4-yl)ethyl)benzamide;
5-(hexahydropyrrolo[3,4-c]pyrrol-2(1H)-yl)-2-methyl-N—((R)-1-(2-(1-methyl-1H-pyrazol-4-yl)quinolin-4-yl)ethyl)benzamide;
2-methyl-N—((R)-1-(2-(1-methyl-1H-pyrazol-4-yl)quinolin-4-yl)ethyl)-5-(5-methylhexahydropyrrolo[3,4-c]pyrrol-2(1H)-yl)benzamide;
methyl(R)-5-(4-(1-(5-(aminomethyl)-2-methylbenzamido)ethyl)quinolin-2-yl)-1-methyl-1H-pyrrole-3-carboxylate;
(R)—N-methyl-N-(3-methyl-4-((1-(2-(1-methyl-1H-pyrazol-4-yl)quinolin-4-yl)ethyl)carbamoyl)benzyl)thiazole-4-carboxamide;
(R)—N-methyl-N-(3-methyl-4-((1-(2-(1-methyl-1H-pyrazol-4-yl)quinolin-4-yl)ethyl)carbamoyl)benzyl)-1H-imidazole-2-carboxamide;
(R)-2-methyl-4-((methyl(thiazol-4-ylmethyl)amino)methyl)-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-((N-(thiazol-4-ylmethyl)acetamido)methyl)benzamide;
(R)-2-methyl-N-(1-(2-(1-methyl-1H-pyrazol-4-yl)quinolin-4-yl)ethyl)-4-((N-(thiazol-4-ylmethyl)methylsulfonamido)methyl)benzamide;
N-(1-(3-methyl-4-(((R)-1-(2-(1-methyl-1H-pyrazol-4-yl)quinolin-4-yl)ethyl)carbamoyl)phenyl)ethyl)thiazole-4-carboxamide;
N-(1-(3-methyl-4-(((R)-1-(2-(1-methyl-1H-pyrazol-4-yl)quinolin-4-yl)ethyl)carbamoyl)phenyl)ethyl)-1H-imidazole-2-carboxamide;
(R)-2-methyl-N4-((1-methyl-1H-imidazol-2-yl)methyl)-N¹-(1-(2-(1-methyl-1H-pyrazol-4-yl)quinolin-4-yl)ethyl)terephthalamide;
(R)—N-(4-methyl-3-((1-(2-(1-methyl-1H-pyrazol-4-yl)quinolin-4-yl)cyclopropyl)carbamoyl)phenyl)piperidine-2-carboxamide;
5-(4-(2-hydroxyethyl)piperazin-1-yl)-2-methyl-N-(1-(2-(1-methyl-1H-pyrazol-4-yl)quinolin-4-yl)cyclopropyl)benzamide;
(R)-2-methyl-N-(1-(2-(1-methyl-1H-pyrazol-4-yl)quinolin-4-yl)ethyl)-4-((thiazol-4-ylmethoxy)methyl)benzamide;
2-methyl-N-(1-(2-(3-methyl-1H-pyrazol-5-yl)quinolin-4-yl)cyclopropyl)-5-(4-methylpiperazin-1-yl)benzamide;
N,N-dimethyl-5-(4-(1-(2-methyl-5-(piperazin-1-yl)benzamido)cyclopropyl)naphthalen-2-yl)-1H-pyrrole-2-carboxamide;
N,N-dimethyl-5-(4-(1-(2-methyl-5-(4-methylpiperazin-1-yl)benzamido)cyclopropyl)naphthalen-2-yl)-1H-pyrrole-3-carboxamide;
rel-5-((1S,4S)-2,5-diazabicyclo[2.2.1]heptan-2-yl)-2-methyl-N-(1-(2-(1-methyl-1H-pyrazol-4-yl)quinolin-4-yl)cyclopropyl)benzamide, ENT-1;
rel-5-((1S,4S)-2,5-diazabicyclo[2.2.1]heptan-2-yl)-2-methyl-N-(1-(2-(1-methyl-1H-pyrazol-4-yl)quinolin-4-yl)cyclopropyl)benzamide, ENT-2;
2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-1;
2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-2;
(R)-2-methyl-4-((((1-methyl-1H-imidazol-2-yl)methyl)thio)methyl)-N-(1-(2-(1-methyl-1H-pyrazol-4-yl)quinolin-4-yl)ethyl)benzamide;
2-methyl-4-((((1-methyl-1H-imidazol-2-yl)methyl)sulfinyl)methyl)-N—((R)-1-(2-(1-methyl-1H-pyrazol-4-yl)quinolin-4-yl)ethyl)benzamide;
2-(2,5-diazabicyclo[2.2.2]octan-2-yl)-5-methyl-N-(1-(2-(1-methyl-1H-pyrazol-4-yl)quinolin-4-yl)cyclopropyl)isonicotinamide;
2-(3,8-diazabicyclo[3.2.1]octan-3-yl)-5-methyl-N-(1-(2-(1-methyl-1H-pyrazol-4-yl)quinolin-4-yl)cyclopropyl)isonicotinamide;
5-methyl-N-(1-(2-(1-methyl-1H-pyrazol-4-yl)quinolin-4-yl)cyclopropyl)-2-(5-methyl-2,5-diazabicyclo[2.2.2]octan-2-yl)isonicotinamide;
5-methyl-N-(1-(2-(1-methyl-1H-pyrazol-4-yl)quinolin-4-yl)cyclopropyl)-2-(8-methyl-3,8-diazabicyclo[3.2.1]octan-3-yl)isonicotinamide;
2-methyl-N-(1-(2-(1-methyl-1H-pyrazol-4-yl)quinolin-4-yl)cyclopropyl)-4-(5-methyl-2,5-diazabicyclo[2.2.1]heptan-2-yl)benzamide;
5-(3,6-diazabicyclo[3.1.1]heptan-3-yl)-2-methyl-N-(1-(2-(1-methyl-1H-pyrazol-4-yl)quinolin-4-yl)cyclopropyl)benzamide;
2-methyl-N-(1-(2-(1-methyl-1H-pyrazol-4-yl)quinolin-4-yl)cyclopropyl)-5-(6-methyl-3,6-diazabicyclo[3.1.1]heptan-3-yl)benzamide;
(R)-2-methyl-4-(((1-methyl-1H-imidazol-2-yl)methoxy)methyl)-N-(1-(2-(1-methyl-1H-pyrazol-4-yl)quinolin-4-yl)ethyl)benzamide;
N,N-dimethyl-5-(4-(1-(2-methyl-5-(4-methylpiperazin-1-yl)benzamido)cyclopropyl)naphthalen-2-yl)-1H-pyrrole-2-carboxamide;
N-(1-(2-(1-(2-methoxyethyl)-1H-pyrazol-4-yl)quinolin-4-yl)cyclopropyl)-2-methyl-5-(4-methylpiperazin-1-yl)benzamide;
2-methyl-5-(4-methylpiperazin-1-yl)-N-(1-(2-(1-(oxetan-3-yl)-1H-pyrazol-4-yl)quinolin-4-yl)cyclopropyl)benzamide;
2-methyl-N-(3-(3-(1-methyl-1H-pyrazol-4-yl)naphthalen-1-yl)oxetan-3-yl)-5-(4-methylpiperazin-1-yl)benzamide;
5-(4-(2-methoxyethyl)piperazin-1-yl)-2-methyl-N-(1-(2-(1-methyl-1H-pyrazol-4-yl)quinolin-4-yl)cyclopropyl)benzamide;
2-methyl-N-(1-(2-(1-methyl-1H-pyrazol-4-yl)quinolin-4-yl)cyclopropyl)-5-(4-(oxetan-3-ylmethyl)piperazin-1-yl)benzamide;
(R)-2-methyl-N-(1-(2-(1-methyl-1H-pyrazol-4-yl)quinolin-4-yl)ethyl)-4-(2-((5-methylthiazol-4-yl)amino)-2-oxoethyl)benzamide;
N-(1-(2-(1H-pyrazol-4-yl)quinolin-4-yl)cyclopropyl)-2-methyl-5-(4-methylpiperazin-1-yl)benzamide;
N-(1-(2-(1H-pyrazol-3-yl)quinolin-4-yl)cyclopropyl)-2-methyl-5-(4-methylpiperazin-1-yl)benzamide;
4-(1-hydroxy-2-oxo-2-(thiazol-4-ylamino)ethyl)-2-methyl-N—((R)-1-(2-(1-methyl-1H-pyrazol-4-yl)quinolin-4-yl)ethyl)benzamide;
rel-2-methyl-N-(1-(2-(1-methyl-1H-pyrazol-4-yl)quinolin-4-yl)cyclopropyl)-5-((1R,4R)-5-methyl-2,5-diazabicyclo[2.2.1]heptan-2-yl)benzamide, ENT-1;
rel-2-methyl-N-(1-(2-(1-methyl-1H-pyrazol-4-yl)quinolin-4-yl)cyclopropyl)-5-((1R,4R)-5-methyl-2,5-diazabicyclo[2.2.1]heptan-2-yl)benzamide, ENT-2;
5-methyl-N-(1-(2-(1-methyl-1H-pyrazol-4-yl)quinolin-4-yl)cyclopropyl)-2-((3aR,6aS)-5-methylhexahydropyrrolo[3,4-c]pyrrol-2(1H)-yl)isonicotinamide;
N-(1-(2-(1-(difluoromethyl)-1H-pyrazol-4-yl)quinolin-4-yl)cyclopropyl)-2-methyl-5-(piperazin-1-yl)benzamide;
(S)-2-methyl-N-(1-(2-(1-methyl-1H-pyrazol-4-yl)quinolin-4-yl)cyclopropyl)-5-(3-methylpiperazin-1-yl)benzamide;
N-(1-(2-(1-ethyl-1H-pyrazol-4-yl)quinolin-4-yl)cyclopropyl)-2-methyl-5-(piperazin-1-yl)benzamide;
N-(1-(2-(isothiazol-5-yl)quinolin-4-yl)cyclopropyl)-2-methyl-5-(piperazin-1-yl)benzamide;
2-methyl-5-(piperazin-1-yl)-N-(1-(2-(thiazol-5-yl)quinolin-4-yl)cyclopropyl)benzamide;
2-methyl-N—((R)-1-(2-(1-methyl-1H-pyrazol-4-yl)quinolin-4-yl)ethyl)-4-(2,2,2-trifluoro-1-((thiazol-4-ylmethyl)amino)ethyl)benzamide;
(R)-2-methyl-N-(1-(2-(1-methyl-1H-pyrazol-4-yl)quinolin-4-yl)cyclopropyl)-5-(3-methylpiperazin-1-yl)benzamide;
(S)-5-(3,4-dimethylpiperazin-1-yl)-2-methyl-N-(1-(2-(1-methyl-1H-pyrazol-4-yl)quinolin-4-yl)cyclopropyl)benzamide;
2-methyl-N-(1-(2-(1-methyl-1H-pyrazol-4-yl)quinolin-4-yl)cyclopropyl)-4-(2-oxo-2-(thiazol-4-ylamino)ethyl)benzamide;
(R)-2-methyl-N1-(1-(2-(1-methyl-1H-pyrazol-4-yl)quinolin-4-yl)ethyl)-N4-(1-(thiazol-4-yl)cyclopropyl)terephthalamide;
5-(4-ethylpiperazin-1-yl)-2-methyl-N-(1-(2-(1-methyl-1H-pyrazol-4-yl)quinolin-4-yl)cyclopropyl)benzamide;
N-(1-(2-(1-(2-methoxyethyl)-1H-pyrazol-4-yl)quinolin-4-yl)cyclopropyl)-2-methyl-5-(piperazin-1-yl)benzamide;
(R)-2-methyl-N1-(1-(2-(1-methyl-1H-pyrazol-4-yl)quinolin-4-yl)ethyl)-N4-((5-methyloxazol-4-yl)methyl)terephthalamide;
5-(4-isopropylpiperazin-1-yl)-2-methyl-N-(1-(2-(1-methyl-1H-pyrazol-4-yl)quinolin-4-yl)cyclopropyl)benzamide;
5-(4-(cyclopropylmethyl)piperazin-1-yl)-2-methyl-N-(1-(2-(1-methyl-1H-pyrazol-4-yl)quinolin-4-yl)cyclopropyl)benzamide;
(R)-5-(3,4-dimethylpiperazin-1-yl)-2-methyl-N-(1-(2-(1-methyl-1H-pyrazol-4-yl)quinolin-4-yl)cyclopropyl)benzamide;
rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzarmide, ENT-1;
rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-2
4-(2-methoxy-3-(pyridin-2-yl)propyl)-2-methyl-N-(1-(2-(1-methyl-1H-pyrazol-4-yl)quinolin-4-yl)cyclopropyl)benzamide;
4-(2-hydroxy-3-(pyridin-2-yl)propyl)-2-methyl-N-(1-(2-(1-methyl-1H-pyrazol-4-yl)quinolin-4-yl)cyclopropyl)benzamide;
2-methyl-N-(1-(2-(1-(2-(methylsulfonyl)ethyl)-1H-pyrazol-4-yl)quinolin-4-yl)cyclopropyl)-4-((thiazol-4-ylmethoxy)methyl)benzamide;
N-(2-(dimethylamino)ethyl)-N-methyl-5-(4-(1-(2-methyl-4-((thiazol-4-ylmethoxy)methyl)benzamido)cyclopropyl)quinolin-2-yl)-1H-pyrazole-3-carboxamide;
4-(((5-(hydroxymethyl)thiazol-4-yl)methoxy)methyl)-2-methyl-N-(1-(2-(1-methyl-1H-pyrazol-4-yl)quinolin-4-yl)cyclopropyl)benzamide;
4-(2-methoxy-1-(thiazol-4-ylmethoxy)ethyl)-2-methyl-N—((R)-1-(2-(1-methyl-1H-pyrazol-4-yl)quinolin-4-yl)ethyl)benzamide;
4-((R*)-2-methoxy-1-(thiazol-4-ylmethoxy)ethyl)-2-methyl-N—((R)-1-(2-(1-methyl-1H-pyrazol-4-yl)quinolin-4-yl)ethyl)benzamide, ENT-1:
4-((R*)-2-methoxy-1-(thiazol-4-ylmethoxy)ethyl)-2-methyl-N—((R)-1-(2-(1-methyl-1H-pyrazol-4-yl)quinolin-4-yl)ethyl)benzamide, ENT-2;
rel-(R)-4-(2-methoxy-1-(thiazol-4-ylmethoxy)ethyl)-2-methyl-N-(1-(7-(1-methyl-1H-pyrazol-4-yl)quinolin-5-yl)cyclopropyl)benzamide, ENT-1;
rel-(R)-4-(2-methoxy-1-(thiazol-4-ylmethoxy)ethyl)-2-methyl-N-(1-(7-(1-methyl-1H-pyrazol-4-yl)quinolin-5-yl)cyclopropyl)benzamide, ENT-2;
rel-(R)-5-(4-(1-(4-(2-methoxy-1-(thiazol-4-ylmethoxy)ethyl)-2-methylbenzamido)cyclopropyl)quinolin-2-yl)-N,N-dimethyl-1H-pyrazole-3-carboxamide, ENT-1;
rel-(R)-5-(4-(1-(4-(2-methoxy-1-(thiazol-4-ylmethoxy)ethyl)-2-methylbenzamido)cyclopropyl)quinolin-2-yl)-N,N-dimethyl-1H-pyrazole-3-carboxamide, ENT-2;
rel-(R)-4-(2-methoxy-1-(oxazol-4-ylmethoxy)ethyl)-2-methyl-N-(1-(2-(1-methyl-1H-pyrazol-4-yl)quinolin-4-yl)cyclopropyl)benzamide, ENT-1;
rel-(R)-4-(2-methoxy-1-(oxazol-4-ylmethoxy)ethyl)-2-methyl-N-(1-(2-(1-methyl-1H-pyrazol-4-yl)quinolin-4-yl)cyclopropyl)benzamide, ENT-2;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclopropyl)benzamide, ENT-1;
2-methyl-N-(1-(2-(1-((methylsulfonyl)methyl)-1H-pyrazol-4-yl)quinolin-4-yl)cyclopropyl)-4-((thiazol-4-ylmethoxy)methyl)benzamide;
N-(1-(2-(1-(2-(dirnethylamino)-2-oxoethyl)-1H-pyrazol-4-yl)quinolin-4-yl)cyclopropyl)-2-methyl-4-((thiazol-4-ylmethoxy)methyl)benzamide;
N,N-dimethyl-5-(4-(1-(2-methyl-4-((thiazol-4-ylmethoxy)methyl)benzamido)cyclopropyl) quinolin-2-yl)-1H-pyrazole-3-carboxamide;
2-methyl-N-(1-(2-(1-methyl-1H-pyrazol-4-yl)quinolin-4-yl)cyclopropyl)-4-((oxazol-4-ylmethoxy)methyl)benzamide;
2-methyl-N-(1-(7-(1-methyl-1H-pyrazol-4-yl)quinolin-5-yl)cyclopropyl)-4-((oxazol-4-ylmethoxy)methyl)benzamide;
N,N-dimethyl-5-(4-(1-(2-methyl-4-((oxazol-4-ylmethoxy)methyl)benzamido)cyclopropyl) quinolin-2-yl)-1H-pyrazole-3-carboxamide;
N-methyl-5-(4-(1-(2-methyl-4-((thiazol-4-ylmethoxy)methyl)benzamido)cyclopropyl) quinolin-2-yl)-1H-pyrazole-3-carboxamide;
2-methyl-N-(1-(2-methyl-7-(1-methyl-1H-pyrazol-4-yl)quinolin-5-yl)cyclopropyl)-4-((oxazol-4-ylmethoxy)methyl)benzamide;
5-(4-(1-(2-methyl-4-((thiazol-4-ylmethoxy)methyl)benzamido)cyclopropyl)quinolin-2-yl)-1H-pyrazole-3-carboxamide;
N,N-dimethyl-5-(5-(1-(2-methyl-4-((oxazol-4-ylmethoxy)methyl)benzamido)cyclopropyl) quinolin-7-yl)-1H-pyrazole-3-carboxamide;
N,N-dimethyl-5-(5-(1-(2-methyl-4-((thiazol-4-ylmethoxy)methyl)benzamido)cyclopropyl) quinolin-7-yl)-1H-pyrazole-3-carboxamide;
N-(1-(2-(1-(2-amino-2-oxoethyl)-1H-pyrazol-4-yl)quinolin-4-yl)cyclopropyl)-2-methyl-4-((thiazol-4-ylmethoxy)methyl)benzamide;
4-(2-methoxy-1-(thiazol-4-ylmethoxy)ethyl)-2-methyl-N-(1-(2-methyl-7-(1-methyl-1H-pyrazol-4-yl)quinolin-5-yl)cyclopropyl)benzamide;
N-(1-(2-(2-hydroxypropan-2-yl)-7-(1-methyl-1H-pyrazol-4-yl)quinolin-5-yl)cyclopropyl)-2-methyl-4-((thiazol-4-ylmethoxy)methyl)benzamide;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclopropyl)benzamide, ENT-1;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclopropyl)benzamide, ENT-2;
N-(2-hydroxy-2-methylpropyl)-N-methyl-5-(4-(1-(2-methyl-4-((thiazol-4-ylmethoxy)methyl)benzamido)cyclopropyl)quinolin-2-yl)-1H-pyrazole-3-carboxamide;
N-(1-(2-hydroxy-7-(1-methyl-1H-pyrazol-4-yl)quinolin-5-yl)cyclopropyl)-2-methyl-4-((thiazol-4-ylmethoxy)methyl)benzamide;
rel-(R)-5-(5-(1-(4-(2-methoxy-1-(oxazol-4-ylmethoxy)ethyl)-2-methylbenzamido)cyclopropyl)quinolin-7-yl)-N,N-dimethyl-1H-pyrazole-3-carboxamide, ENT-1;
rel-(R)-5-(5-(1-(4-(2-methoxy-1-(oxazol-4-ylmethoxy)ethyl)-2-methylbenzamido)cyclopropyl)quinolin-7-yl)-N,N-dimethyl-1H-pyrazole-3-carboxamide, ENT-2;
rac-(R)-5-(5-(1-(4-(2-methoxy-1-(thiazol-4-ylmethoxy)ethyl)-2-methylbenzamido)cyclopropyl)quinolin-7-yl)-N,N-dimethyl-1H-pyrazole-3-carboxamide;
N,N-dimethyl3-(4-{4-[1-(4-{[(1,3-thiazol-4-yl)methoxy]methyl}-2-toluoylamino)cyclopropyl]-2-quinolyl}-1-pyrazolyl)propionamide;
N,N-dimethyl2-methyl-2-(4-{4-[1-(4-{[(1,3-thiazol-4-yl)methoxy]methyl}-2-toluoylamino)cyclopropyl]-2-quinolyl}-1-pyrazolyl)propionamide;
N-ethyl-N-methyl(4-{4-[1-(4-{[(1,3-thiazol-4-yl)methoxy]methyl}-2-toluoylamino)cyclopropyl]-2-quinolyl}-1-pyrazolyl)acetamide;
rel-(R)-4-(2-methoxy-1-(oxazol-4-ylmethoxy)ethyl)-2-methyl-N-(1-(2-methyl-7-(1-methyl-1H-pyrazol-4-yl)quinolin-5-yl)cyclopropyl)benzamide, ENT-1; and N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide;
   or pharmaceutically acceptable salts thereof.

E42 A compound of embodiment E1 which is selected from the group consisting of:
(R)-2-methyl-N-(1-(2-(1-methyl-1H-pyrazol-4-yl)quinolin-4-yl)ethyl)-4-(((thiazol-4-ylmethyl)amino)methyl)benzamide;
2-methyl-N-(1-(3-(1-methyl-1H-pyrazol-4-yl)naphthalen-1-yl)cyclopropyl)-5-(4-methylpiperazin-1-yl)benzamide;
2-methyl-N-(1-(2-(1-methyl-1H-pyrazol-4-yl)quinolin-4-yl)cyclopropyl)-5-(4-methylpiperazin-1-yl)benzamide;
(2-methyl-N-(1-(2-(1-methyl-1H-pyrazol-4-yl)quinolin-4-yl)cyclopropyl)-5-(5-methylhexahydropyrrolo[3,4-c]pyrrol-2(1H)-yl)benzamide;
(R)-2-methyl-N-(1-(2-(1-methyl-1H-pyrazol-4-yl)quinolin-4-yl)ethyl)-4-(2-oxo-2-(thiazol-4-ylamino)ethyl)benzamide;
(R)-2-methyl-N1-(1-(2-(i-methyl-1H-pyrazol-4-yl)quinolin-4-yl)ethyl)-N4-((5-methylthiazol-4-yl)methyl)terephthalamide;
2-methyl-N-(1-(2-(1-methyl-1H-pyrazol-4-yl)quinolin-4-yl)cyclopropyl)-5-((1R,5S)-6-methyl-3,6-diazabicyclo[3.1.1]heptan-3-yl)benzamide;
5-((1R,5S)-3,6-diazabicyclo[3.1.1]heptan-3-yl)-2-methyl-N-(1-(2-(1-methyl-1H-pyrazol-4-yl)quinolin-4-yl)cyclopropyl)benzamide;
2-methyl-5-(4-methylpiperazin-1-yl)-N-(1-(2-(1-(oxetan-3-yl)-1H-pyrazol-4-yl)quinolin-4-yl)cyclopropyl)benzamide;
5-(4-(2-hydroxyethyl)piperazin-1-yl)-2-methyl-N-(1-(2-(1-methyl-1H-pyrazol-4-yl)quinolin-4-yl)cyclopropyl)benzamide;
5-(hexahydropyrrolo[3,4-c]pyrrol-2(1H)-yl)-2-methyl-N-(1-(2-(1-methyl-1H-pyrazol-4-yl)quinolin-4-yl)cyclopropyl)benzamide;
2-methyl-N-(1-(2-(1-methyl-1H-pyrazol-4-yl)quinolin-4-yl)cyclopropyl)-5-(piperazin-1-yl)benzamide;
(R)-2-methyl-N-(1-(2-(1-methyl-1H-pyrazol-4-yl)quinolin-4-yl)ethyl)-4-((thiazol-4-ylmethoxy)methyl)benzamide;
(R)-2-methyl-N-(1-(2-(1-methyl-1H-pyrazol-4-yl)quinolin-4-yl)ethyl)-4-(2-((5-methylthiazol-4-yl)amino)-2-oxoethyl)benzamide;
(R)-2-methyl-4-(((1-methyl-1H-imidazol-2-yl)methoxy)methyl)-N-(1-(2-(1-methyl-1H-pyrazol-4-yl)quinolin-4-yl)ethyl)benzamide;
2-methyl-4-((((1-methyl-1H-imidazol-2-yl)methyl)sulfinyl)methyl)-N—((R)-1-(2-(1-methyl-1H-pyrazol-4-yl)quinolin-4-yl)ethyl)benzamide;
5-methyl-N-(1-(2-(1-methyl-1H-pyrazol-4-yl)quinolin-4-yl)cyclopropyl)-2-((3aR,6aS)-5-methylhexahydropyrrolo[3,4-c]pyrrol-2(1H)-yl)isonicotinamide;
2-methyl-N-(1-(2-(1-methyl-1H-pyrazol-4-yl)quinolin-4-yl)cyclopropyl)-4-(5-methyl-2,5-diazabicyclo[2.2.1]heptan-2-yl)benzamide;
5-methyl-N-(1-(2-(1-methyl-1H-pyrazol-4-yl)quinolin-4-yl)cyclopropyl)-2-(5-methyl-2,5-diazabicyclo[2.2.2]octan-2-yl)isonicotinamide;
5-methyl-N-(1-(2-(1-methyl-1H-pyrazol-4-yl)quinolin-4-yl)cyclopropyl)-2-(8-methyl-3,8-diazabicyclo[3.2.1]octan-3-yl)isonicotinamide;
4-methyl-N—((R)-1-(2-(1-methyl-1H-pyrazol-4-yl)quinolin-4-yl)ethyl)-6-(1-(thiazol-4-ylmethoxy)ethyl)nicotinamide;
2-methyl-N-(1-(2-(1-methyl-1H-pyrazol-4-yl)quinolin-4-yl)cyclopropyl)-4-((thiazol-4-ylmethoxy)methyl)benzamide;
N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide;
N,N-dimethyl-5-(4-(1-(2-methyl-4-((thiazol-4-ylmethoxy)methyl)benzamido)cyclopropyl) quinolin-2-yl)-1H-pyrazole-3-carboxamide;
N,N-dimethyl-5-(5-(1-(2-methyl-4-((oxazol-4-ylmethoxy)methyl)benzamido)cyclopropyl) quinolin-7-yl)-1H-pyrazole-3-carboxamide; and
N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide;
   or a pharmaceutically acceptable salt thereof.

E43 A compound of embodiment E1 which is selected from the group consisting of:
4-((((1H-imidazol-2-yl)methyl)amino)methyl)-2-methyl-N-(1-(3-(1-methyl-1H-pyrazol-4-yl)naphthalen-1-yl)ethyl)benzamide;
2-methyl-N-(1-(3-(1-methyl-1H-pyrazol-4-yl)naphthalen-1-yl)ethyl)-4-(((thiazol-4-ylmethyl)amino)methyl)benzamide;
(R)-2-methyl-N-(1-(2-(1-methyl-1H-pyrazol-4-yl)quinolin-4-yl)ethyl)-4-(((thiazol-4-ylmethyl)amino)methyl)benzamide;
N-(3-methyl-4-((1-(3-(1-methyl-1H-pyrazol-4-yl)naphthalen-1-yl)ethyl)carbamoyl)benzyl)thiazole-4-carboxamide
N-(3-methyl-4-((1-(3-(1-methyl-1H-pyrazol-4-yl)naphthalen-1-yl)ethyl)carbamoyl)benzyl)-1H-imidazole-2-carboxamide;
(R)-2-(3-methyl-4-((1-(2-(1-methyl-1H-pyrazol-4-yl)quinolin-4-yl)ethyl)carbamoyl)phenyl)acetic acid;
(R)-2-methyl-N1-(1-(2-(1-methyl-1H-pyrazol-4-yl)quinolin-4-yl)ethyl)-N4-(thiazol-4-ylmethyl)terephthalamide;
2-methyl-N1-((R)-1-(2-(1-methyl-1H-pyrazol-4-yl)quinolin-4-yl)ethyl)-N4-(1-(thiazol-4-yl)ethyl)terephthalamide;
(R)-2-methyl-N1-(1-(2-(i-methyl-1H-pyrazol-4-yl)quinolin-4-yl)ethyl)-N4-((5-methylthiazol-4-yl)methyl)terephthalamide;
(R)-2-methyl-N1-(1-(2-(1-methyl-1H-pyrazol-4-yl)quinolin-4-yl)ethyl)-N4-((2-methylthiazol-4-yl)methyl)terephthalamide;
(R)-N4-(1-(1H-imidazol-2-yl)cyclopropyl)-2-methyl-N1-(1-(2-(1-methyl-1H-pyrazol-4-yl)quinolin-4-yl)ethyl)terephthalamide;
(R)—N4,2-dimethyl-N¹-(1-(2-(1-methyl-1H-pyrazol-4-yl)quinolin-4-yl)ethyl)-N⁴-(thiazol-4-ylmethyl)terephthalamide;
(R)-N4-(2-(1H-imidazol-2-yl)propan-2-yl)-2-methyl-N1-(1-(2-(1-methyl-1H-pyrazol-4-yl)quinolin-4-yl)ethyl)terephthalamide;
(R)-N4-((1H-imidazol-2-yl)methyl)-2-methyl-N1-(1-(2-(1-methyl-1H-pyrazol-4-yl)quinolin-4-yl)ethyl)terephthalamide;
N4-(1-(1H-imidazol-2-yl)ethyl)-2-methyl-N1-((R)-1-(2-(1-methyl-1H-pyrazol-4-yl)quinolin-4-yl)ethyl)terephthalamide;
2-methyl-N⁴-((1-methyl-1H-imidazol-2-yl)methyl)-N¹-(1-(2-(1-methyl-1H-pyrazol-4-yl)quinolin-4-yl)cyclopropyl) terephthalamide;
2-methyl-N¹-(1-(2-(1-methyl-1H-pyrazol-4-yl)quinolin-4-yl)cyclopropyl)-N⁴-(thiazol-4-ylmethyl)terephthalamide;
(R)-2-methyl-N4-((1-methyl-1H-imidazol-2-yl)methyl)-N¹-(1-(2-(1-methyl-1H-pyrazol-4-yl)quinolin-4-yl)ethyl)terephthalamide;
2-methyl-N-(1-(2-(1-methyl-1H-pyrazol-4-yl)quinolin-4-yl)cyclopropyl)-4-(((thiazol-4-ylmethyl)amino)methyl)benzamide;
(R)—N-(1-(3-methyl-4-((1-(2-(1-methyl-1H-pyrazol-4-yl)quinolin-4-yl)ethyl) carbamoyl)phenyl)cyclopropyl)thiazole-4-carboxamide;
(R)—N-(1-(3-methyl-4-((1-(2-(1-methyl-1H-pyrazol-4-yl)quinolin-4-yl)ethyl)carbamoyl)phenyl)cyclopropyl)-1H-imidazole-2-carboxamide;
(R)—N-(2-(3-methyl-4-((1-(2-(1-methyl-1H-pyrazol-4-yl)quinolin-4-yl)ethyl) carbamoyl)phenyl)propan-2-yl)thiazole-4-carboxamide;
(R)—N-(2-(3-methyl-4-((1-(2-(1-methyl-1H-pyrazol-4-yl)quinolin-4-yl)ethyl) carbamoyl)phenyl)propan-2-yl)-1H-imidazole-2-carboxamide;
(R)-2-methyl-N-(1-(2-(1-methyl-1H-pyrazol-4-yl)quinolin-4-yl)ethyl)-4-(2-((thiazol-4-ylmethyl)amino)propan-2-yl)benzamide;
N-(1-(3-methyl-4-(((R)-1-(2-(1-methyl-1H-pyrazol-4-yl)quinolin-4-yl)ethyl)carbamoyl)phenyl)ethyl)thiazole-4-carboxamide;
N-(1-(3-methyl-4-(((R)-1-(2-(1-methyl-1H-pyrazol-4-yl)quinolin-4-yl)ethyl)carbamoyl)phenyl)ethyl)-1H-imidazole-2-carboxamide;
(R)-2-methyl-4-((methyl(thiazol-4-ylmethyl)amino)methyl)-N-(1-(2-(1-methyl-1H-pyrazol-4-yl)quinolin-4-yl)ethyl)benzamide;
(R)-2-methyl-4-((((1-methyl-1H-imidazol-5-yl)methyl)amino)methyl)-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-(2-oxo-2-(thiazol-4-ylamino)ethyl)benzamide;
(R)-2-methyl-N-(1-(2-(1-methyl-1H-pyrazol-4-yl)quinolin-4-yl)ethyl)-4-(2-oxo-2-(thiazol-5-ylamino)ethyl)benzamide;
(R)-2-methyl-N-(1-(2-(1-methyl-1H-pyrazol-4-yl)quinolin-4-yl)ethyl)-4-(2-oxo-2-(thiazol-2-ylamino)ethyl)benzamide;
(R)-4-(2-((1H-imidazol-2-yl)amino)-2-oxoethyl)-2-methyl-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-(2-(methylamino)-2-oxoethyl)benzamide;
(R)-4-(2-(dimethylamino)-2-oxoethyl)-2-methyl-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-((N-(thiazol-4-ylmethyl)acetamido)methyl)benzamide;
(R)-2-methyl-N-(1-(2-(1-methyl-1H-pyrazol-4-yl)quinolin-4-yl)ethyl)-4-((N-(thiazol-4-ylmethyl)methylsulfonamido)methyl)benzamide;
(R)—N-methyl-N-(3-methyl-4-((1-(2-(1-methyl-1H-pyrazol-4-yl)quinolin-4-yl)ethyl)carbamoyl)benzyl)thiazole-4-carboxamide;
(R)—N-methyl-N-(3-methyl-4-((1-(2-(1-methyl-1H-pyrazol-4-yl)quinolin-4-yl)ethyl)carbamoyl)benzyl)-1H-imidazole-2-carboxamide;
(R)-2-methyl-N-(1-(2-(1-methyl-1H-pyrazol-4-yl)quinolin-4-yl)ethyl)-4-((thiazol-4-ylmethoxy)methyl)benzamide;
(R)-2-methyl-4-((((1-methyl-1H-imidazol-2-yl)methyl)thio)methyl)-N-(1-(2-(1-methyl-1H-pyrazol-4-yl)quinolin-4-yl)ethyl)benzamide;
2-methyl-4-((((1-methyl-1H-imidazol-2-yl)methyl)sulfinyl)methyl)-N—((R)-1-(2-(1-methyl-1H-pyrazol-4-yl)quinolin-4-yl)ethyl)benzamide;
4-methyl-N—((R)-1-(2-(1-methyl-1H-pyrazol-4-yl)quinolin-4-yl)ethyl)-6-(1-(thiazol-4-ylmethoxy)ethyl)nicotinamide;
(R)-2-methyl-4-(((1-methyl-1H-imidazol-2-yl)methoxy)methyl)-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-(2-((5-methylthiazol-4-yl)amino)-2-oxoethyl)benzamide;
4-(1-hydroxy-2-oxo-2-(thiazol-4-ylamino)ethyl)-2-methyl-N—((R)-1-(2-(1-methyl-1H-pyrazol-4-yl)quinolin-4-yl)ethyl)benzamide;
4-methyl-6-(1-((1-methyl-1H-imidazol-2-yl)methoxy)ethyl)-N—((R)-1-(2-(1-methyl-1H-pyrazol-4-yl)quinolin-4-yl)ethyl)nicotinamide;
2-methyl-N-(1-(2-(1-methyl-1H-pyrazol-4-yl)quinolin-4-yl)cyclopropyl)-4-((thiazol-4-ylmethoxy)methyl)benzamide;
2-methyl-N—((R)-1-(2-(1-methyl-1H-pyrazol-4-yl)quinolin-4-yl)ethyl)-4-(2,2,2-trifluoro-1-((thiazol-4-ylmethyl)amino)ethyl)benzamide;
2-methyl-N-(1-(2-(1-methyl-1H-pyrazol-4-yl)quinolin-4-yl)cyclopropyl)-4-(2-oxo-2-(thiazol-4-ylamino)ethyl)benzamide;
(R)-2-methyl-N1-(1-(2-(1-methyl-1H-pyrazol-4-yl)quinolin-4-yl)ethyl)-N4-(1-(thiazol-4-yl)cyclopropyl)terephthalamide;
(R)-2-methyl-N1-(1-(2-(1-methyl-1H-pyrazol-4-yl)quinolin-4-yl)ethyl)-N4-((5-methyloxazol-4-yl)methyl)terephthalamide;
rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-1;
rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-2;
4-(2-methoxy-3-(pyridin-2-yl)propyl)-2-methyl-N-(1-(2-(1-methyl-1H-pyrazol-4-yl)quinolin-4-yl)cyclopropyl)benzamide;
4-(2-hydroxy-3-(pyridin-2-yl)propyl)-2-methyl-N-(1-(2-(1-methyl-1H-pyrazol-4-yl)quinolin-4-yl)cyclopropyl)benzamide;
2-methyl-N-(1-(2-(1-(2-(methylsulfonyl)ethyl)-1H-pyrazol-4-yl)quinolin-4-yl)cyclopropyl)-4-((thiazol-4-ylmethoxy)methyl)benzarmide;
N-(2-(dimethylamino)ethyl)-N-methyl-5-(4-(1-(2-methyl-4-((thiazol-4-ylmethoxy)methyl)benzamido)cyclopropyl)quinolin-2-yl)-1H-pyrazole-3-carboxamide;
4-(((5-(hydroxymethyl)thiazol-4-yl)methoxy)methyl)-2-methyl-N-(1-(2-(1-methyl-1H-pyrazol-4-yl)quinolin-4-yl)cyclopropyl)benzamide;
N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide;
N-(1-(2-(1-(2-amino-2-oxoethyl)-1H-pyrazol-4-yl)quinolin-4-yl)cyclopropyl)-2-methyl-4-((thiazol-4-ylmethoxy)methyl)benzamide;
4-(2-methoxy-1-(thiazol-4-ylmethoxy)ethyl)-2-methyl-N-(1-(2-methyl-7-(1-methyl-1H-pyrazol-4-yl)quinolin-5-yl)cyclopropyl)benzamide;
N-(1-(2-(2-hydroxypropan-2-yl)-7-(1-methyl-1H-pyrazol-4-yl)quinolin-5-yl)cyclopropyl)-2-methyl-4-((thiazol-4-ylmethoxy)methyl)benzamide;
N-(1-(2-hydroxy-7-(1-methyl-1H-pyrazol-4-yl)quinolin-5-yl)cyclopropyl)-2-methyl-4-((thiazol-4-ylmethoxy)methyl)benzamide;
N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide;
4-(2-methoxy-1-(thiazol-4-ylmethoxy)ethyl)-2-methyl-N—((R)-1-(2-(1-methyl-1H-pyrazol-4-yl)quinolin-4-yl)ethyl)benzamide;
4-((R*)-2-methoxy-1-(thiazol-4-ylmethoxy)ethyl)-2-methyl-N—((R)-1-(2-(1-methyl-1H-pyrazol-4-yl)quinolin-4-yl)ethyl)benzamide, ENT-1;
4-((R*)-2-methoxy-1-(thiazol-4-ylmethoxy)ethyl)-2-methyl-N—((R)-1-(2-(1-methyl-1H-pyrazol-4-yl)quinolin-4-yl)ethyl)benzamide, ENT-2;
rel-(R)-4-(2-methoxy-1-(thiazol-4-ylmethoxy)ethyl)-2-methyl-N-(1-(7-(1-methyl-1H-pyrazol-4-yl)quinolin-5-yl)cyclopropyl)benzamide, ENT-1;
rel-(R)-4-(2-methoxy-1-(thiazol-4-ylmethoxy)ethyl)-2-methyl-N-(1-(7-(1-methyl-1H-pyrazol-4-yl)quinolin-5-yl)cyclopropyl)benzamide, ENT-2;
rel-(R)-5-(4-(1-(4-(2-methoxy-1-(thiazol-4-ylmethoxy)ethyl)-2-methylbenzamido)cyclopropyl)quinolin-2-yl)-N,N-dimethyl-1H-pyrazole-3-carboxamide, ENT-1;
rel-(R)-5-(4-(1-(4-(2-methoxy-1-(thiazol-4-ylmethoxy)ethyl)-2-methylbenzamido)cyclopropyl)quinolin-2-yl)-N,N-dimethyl-1H-pyrazole-3-carboxamide, ENT-2;
rel-(R)-4-(2-methoxy-1-(oxazol-4-ylmethoxy)ethyl)-2-methyl-N-(1-(2-(1-methyl-1H-pyrazol-4-yl)quinolin-4-yl)cyclopropyl)benzamide, ENT-1;
rel-(R)-4-(2-methoxy-1-(oxazol-4-ylmethoxy)ethyl)-2-methyl-N-(1-(2-(1-methyl-1H-pyrazol-4-yl)quinolin-4-yl)cyclo-propyl)benzamide, ENT-2;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclopropyl)benzamide, ENT-1;
2-methyl-N-(1-(2-(1-((methylsulfonyl)methyl)-1H-pyrazol-4-yl)quinolin-4-yl)cyclopropyl)-4-((thiazol-4-ylmethoxy)methyl)benzamide;
N,N-dimethyl-5-(4-(1-(2-methyl-4-((thiazol-4-ylmethoxy)methyl)benzamido)cyclopropyl)quinolin-2-yl)-1H-pyrazole-3-carboxamide;
2-methyl-N-(1-(2-(1-methyl-1H-pyrazol-4-yl)quinolin-4-yl)cyclopropyl)-4-((oxazol-4-ylmethoxy)methyl)benzamide;
2-methyl-N-(1-(7-(1-methyl-1H-pyrazol-4-yl)quinolin-5-yl)cyclopropyl)-4-((oxazol-4-ylmethoxy)methyl)benzamide;
N,N-dimethyl-5-(4-(1-(2-methyl-4-((oxazol-4-ylmethoxy)methyl)benzamido)cyclopropyl)quinolin-2-yl)-1H-pyrazole-3-carboxamide;
N-methyl-5-(4-(1-(2-methyl-4-((thiazol-4-ylmethoxy)methyl)benzamido)cyclopropyl)quinolin-2-yl)-1H-pyrazole-3-carboxamide;
2-methyl-N-(1-(2-methyl-7-(1-methyl-1H-pyrazol-4-yl)quinolin-5-yl)cyclopropyl)-4-((oxazol-4-ylmethoxy)methyl)benzamide;
5-(4-(1-(2-methyl-4-((thiazol-4-ylmethoxy)methyl)benzamido)cyclo-propyl)quinolin-2-yl)-1H-pyrazole-3-carboxamide;
N,N-dimethyl-5-(5-(1-(2-methyl-4-((oxazol-4-ylmethoxy)methyl)benzamido)cyclopropyl) quinolin-7-yl)-1H-pyrazole-3-carboxamide;
N,N-dimethyl-5-(5-(1-(2-methyl-4-((thiazol-4-ylmethoxy)methyl)benzamido)cyclopropyl)quinolin-7-yl)-1H-pyrazole-3-carboxamide;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclo-propyl)benzamide, ENT-1;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclo-propyl)benzamide, ENT-2;
N-(2-hydroxy-2-methylpropyl)-N-methyl-5-(4-(1-(2-methyl-4-((thiazol-4-ylmethoxy)methyl)benzamido)cyclopropyl) quinolin-2-yl)-1H-pyrazole-3-carboxamide;
rel-(R)-5-(5-(1-(4-(2-methoxy-1-(oxazol-4-ylmethoxy)ethyl)-2-methylbenzamido)cyclopropyl)quinolin-7-yl)-N,N-dimethyl-1H-pyrazole-3-carboxamide, ENT-1;
rel-(R)-5-(5-(1-(4-(2-methoxy-1-(oxazol-4-ylmethoxy)ethyl)-2-methylbenzamido)cyclopropyl)quinolin-7-yl)-N,N-dimethyl-1H-pyrazole-3-carboxamide, ENT-1;
rel-(R)-5-(5-(1-(4-(2-methoxy-1-(thiazol-4-ylmethoxy)ethyl)-2-methylbenzamido)cyclopropyl)quinolin-7-yl)-N,N-dimethyl-1H-pyrazole-3-carboxamide, ENT-1;
N,N-dimethyl3-(4-{4-[1-(4-{[(1,3-thiazol-4-yl)methoxy]methyl}-2-toluoylamino)cyclopropyl]-2-quinolyl}-1-pyrazolyl)propionamide;
N,N-dimethyl2-methyl-2-(4-{4-[1-(4-{[(1,3-thiazol-4-yl)methoxy]methyl}-2-toluoylamino)cyclopropyl]-2-quinolyl}-1-pyrazolyl)propionamide;
N-ethyl-N-methyl(4-{4-[1-(4-{[(1,3-thiazol-4-yl)methoxy]methyl}-2-toluoylamino)cyclopropyl]-2-quinolyl}-1-pyrazolyl)acetamide; and
rel-(R)-4-(2-methoxy-1-(oxazol-4-ylmethoxy)ethyl)-2-methyl-N-(1-(2-methyl-7-(1-methyl-1H-pyrazol-4-yl)quinolin-5-yl)cyclopropyl)benzamide, ENT-1;
   or a pharmaceutically acceptable salt thereof.

E44 A compound of embodiment E1 which is selected from the group consisting of:
N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide;
N,N-dimethyl-5-(4-(1-(2-methyl-4-((thiazol-4-ylmethoxy)methyl)benzamido)cyclopropyl) quinolin-2-yl)-1H-pyrazole-3-carboxamide;
N,N-dimethyl-5-(5-(1-(2-methyl-4-((oxazol-4-ylmethoxy)methyl)benzamido)cyclopropyl) quinolin-7-yl)-1H-pyrazole-3-carboxamide; and
N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide;
   or a pharmaceutically acceptable salt thereof.

Any of the compounds described in embodiment E41 to E44, or pharmaceutically acceptable salts thereof, may be claimed individually or grouped together with one or more other compounds of embodiments E1 to E40, or pharmaceutically acceptable salts thereof.

E45 A pharmaceutical composition comprising a compound of any one of embodiments E1 to E44, or a pharmaceutically acceptable salt thereof, and at least one pharmaceutically acceptable excipient.

E46 A method for treating a viral infection, comprising administering to a subject in need thereof a therapeutically effective amount of a compound of any one of embodiments E1 to E44, or a pharmaceutically acceptable salt thereof.

E47 A method for treating a viral infection of embodiment E34, wherein the compound of any one of embodiments E1 to E44, or a pharmaceutically acceptable salt thereof, is administered as a single agent.

E48 A method for treating a viral infection of embodiment E46, further comprising administering a therapeutically effective amount of an additional therapeutic agent selected from the list consisting of viral RNA polymerase inhibitors, Mpro inhibitors, nucleoside inhibitors, host factor inhibitors, other PLpro inhibitors and metabolism boosting agents.

E49 A method for treating a viral infection of any one of embodiments E46 to E48, wherein said viral infection is a coronavirus infection.

E50 A method for treating a viral infection of embodiment E49, wherein said coronavirus infection is COVID-19.

E51 A compound of any one of embodiments E1 to E44, for use as a medicament.

E52 A compound of any one embodiments E1 to E44, for use in the treatment of a viral infection.

E53 A compound for use of embodiment E52 wherein said viral infection is a coronavirus infection.

E54 A compound for use of embodiment E53 wherein said coronavirus infection is COVID-19.

E55 Use of a compound of any one of embodiments E1 to E44 for the manufacture of a medicament for the treatment of a viral infection.

E56 Use of a compound of embodiment E55 wherein said viral infection is a coronavirus infection.

E57 Use of a compound of embodiment E56 wherein said coronavirus infection is COVID-19.

E58 A method for the treatment of a disorder mediated by the papain-like protease in a subject, comprising administering to the subject in need thereof a compound of any one of embodiments E1 to E44, or a pharmaceutically acceptable salt thereof, in an amount that is effective for treating the disorder.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein.

Furthermore, each of the embodiments described herein envisions within its scope pharmaceutically acceptable salts of the compounds described herein.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

One of ordinary skill in the art will appreciate that compounds of the present invention include conformational isomers (e.g., cis and trans isomers) and all optical isomers (e.g., enantiomers and diastereomers), racemic, diastereomeric and other mixtures of such isomers, and tautomers thereof, where they may exist. One of ordinary skill in the art will also appreciate that compounds of the invention include solvates, hydrates, isomorphs, polymorphs, esters, salt forms, prodrugs, and isotopically labelled versions thereof, where they may be formed.

As used herein, the term “about” when used to modify a numerically defined parameter means that the parameter may vary by as much as 10% below or above the stated numerical value for that parameter (±10%). For example, a dose of about 5 mg means 5 mg±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I).

“Cyano” refers to a substituent having a carbon atom joined to a nitrogen atom by a triple bond, i.e., —C≡N.

“Hydroxy” refers to an —OH group.

“Nitro” refers to a —NO₂ group “Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein.

“Hydroxyalkyl” refers to an alkyl group, as defined above, wherein from one to all of the hydrogen atoms of the alkyl group are replaced by hydroxy groups. Examples include, but are not limited to, hydroxymethyl, dihydroxymethyl, hydroxyethyl and dihydroxyethyl.

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Alkoxyalkyl” refers to an alkyl group, as defined herein, that is substituted by an alkoxy group, as defined herein. Examples include, but are not limited to, CH₃OCH₂— and CH₃CH₂OCH₂—.

“Alkenyl” refers to an alkyl group, as defined herein, consisting of at least two carbon atoms and at least one carbon-carbon double bond. For example, as used herein, the term “C₂—C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like.

“Alkynyl” refers to an alkyl group, as defined herein, consisting of at least two carbon atoms and at least one carbon-carbon triple bond. Examples include, but are not limited to, ethynyl, 1-propynyl, 2-propynyl, 1-, 2-, or 3-butynyl, and the like.

“Cycloalkyl” refers to a fully saturated hydrocarbon ring system that has the specified number of carbon atoms, which may be a monocyclic, bridged or fused bicyclic, spirocyclic or polycyclic ring system that is connected to the base molecule through a carbon atom of the cycloalkyl ring. Cycloalkyl groups may contain, but are not limited to, 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein. Illustrative examples of cycloalkyl rings include, but are not limited to, the following:

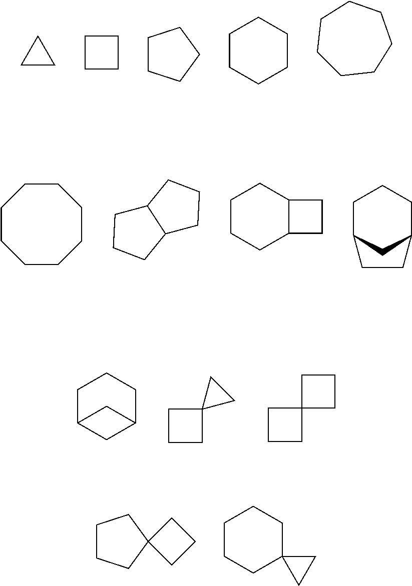

“Cycloalkenyl” refers to a cycloalkyl group, as defined herein, consisting of at least 3 carbon atoms and at least one carbon-carbon double bond. For example, as used herein, the term “C₃-C₈ cycloalkenyl” means a C₃-C₈ cycloalkenyl comprising at least 1 carbon-carbon double bond and which is not aromatic, including, but not limited to, cyclopropenyl, cyclobutenyl, cyclopentenyl, cyclohexenyl, and the like.

“Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms. Heterocycloalkyl rings may be optionally substituted, unsubstituted or substituted, as further defined herein. Such substituents may be present on the heterocyclic ring attached to the base molecule, or on a spirocyclic, bridged or fused ring attached thereto. Heterocycloalkyl rings may include, but are not limited to, 3-10 membered heterocyclyl groups, for example 4-10, 3-8 or 4-8 membered heterocycloalkyl groups, in accordance with the definition herein. Illustrative examples of heterocycloalkyl rings include, but are not limited to a monovalent radical of:

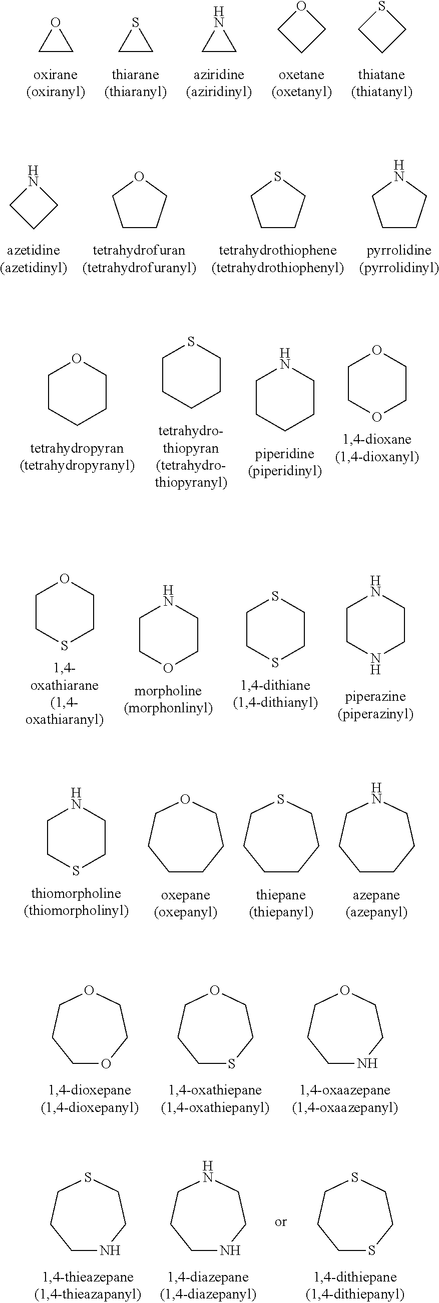

Illustrative examples of bridged, fused and spiro heterocycloalkyl groups include, but are not limited to a monovalent radical of:

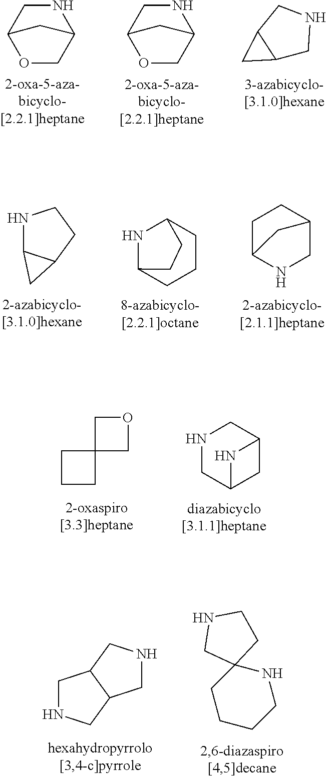

“Heterocycloalkenyl” refers to a heterocycloalkyl group, as defined herein, consisting of at least 3 carbon atoms and at least one carbon-carbon double bond. Heterocycloalkenyl rings may include, but are not limited to, 3-8 membered heterocycloalkenyl groups, for example 3-6 or 5-6 membered heterocycloalkenyl groups, in accordance with the definition herein. Illustrative examples of heterocycloalkenyl rings include, but are not limited to a monovalent radical of:

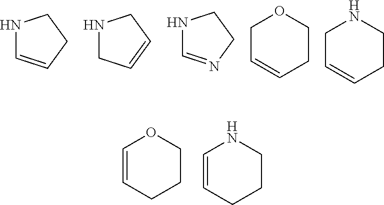

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 10 carbon atoms (C₆-C₁₀ aryl). Fused aryl groups may include an aryl ring (e.g., a phenyl ring) fused to another aryl ring. Examples include, but are not limited to, phenyl, naphthyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzamidazolyl, indazolyl, quinolinyl, isoquinolinyl, purinyl, triazinyl, naphthyridinyl, cinnolinyl, quinazolinyl and quinoxalinyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein. Illustrative examples of monocyclic heteroaryl groups include, but are not limited to a monovalent radical of:

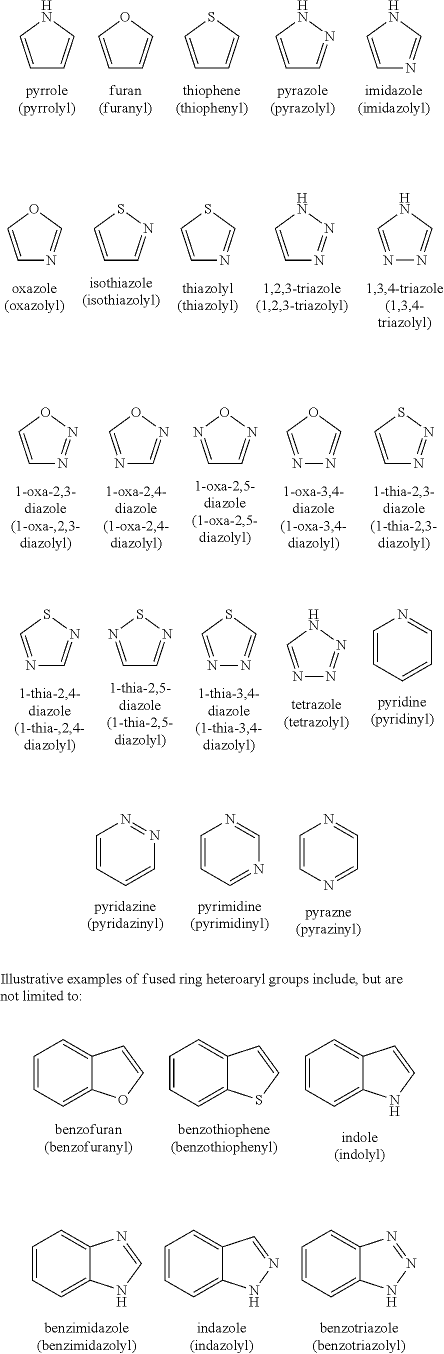

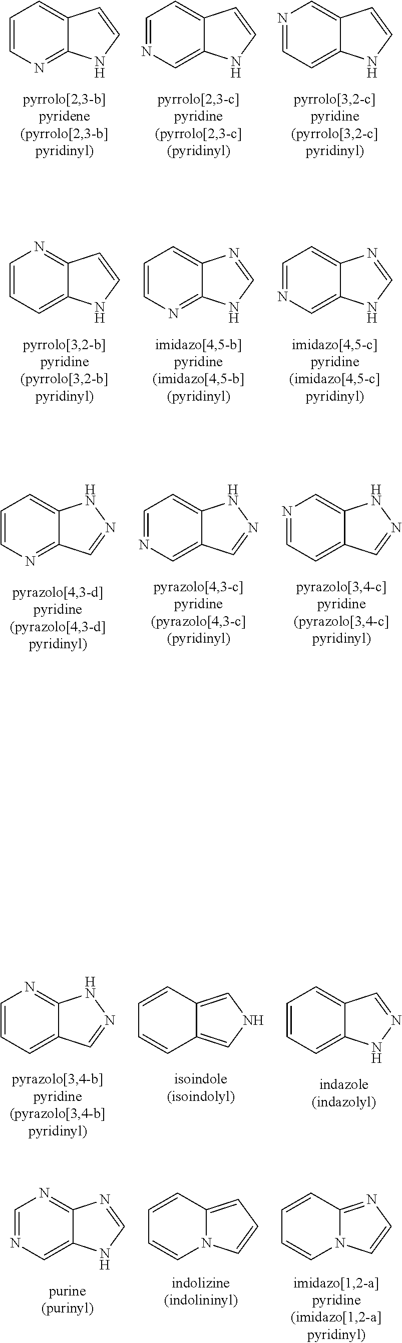

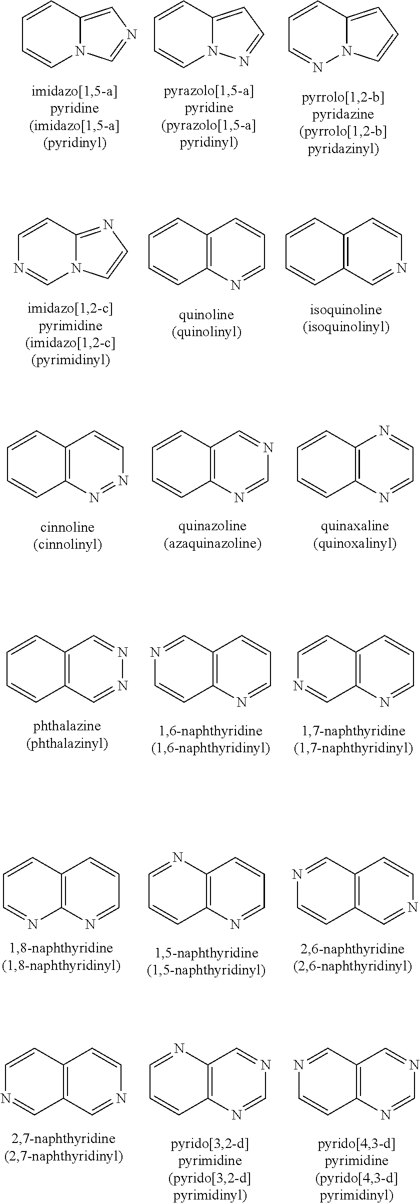

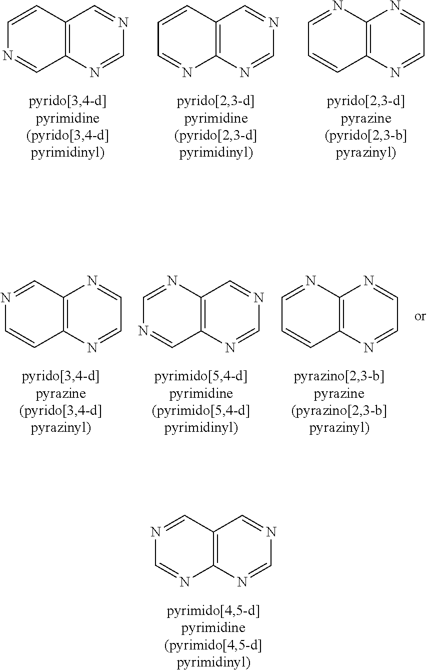

“Amino” refers to a group —NH₂, which is unsubstituted. Where the amino is described as substituted or optionally substituted, the term includes groups of the form —NR^{x}R^{y}, where each of R^{x} and R^{y} is defined as further described herein. For example, “alkylamino” refers to a group —NR^{x}R^{y}, wherein one of R^{x} and R^{y} is an alkyl moiety and the other is H, and “dialkylamino” refers to —NR^{x}R^{y} wherein both of R^{x} and R^{y} are alkyl moieties, where the alkyl moieties have the specified number of carbon atoms (e.g., —NH(C₁-C₄ alkyl) or —N(C₁-C₄ alkyl)₂).

“Aminoalkyl” refers to an alkyl group, as defined above, that is substituted by 1, 2, or 3 amino groups, as defined herein.

The term “pharmaceutically acceptable” means the substance (e.g., the compounds described herein) and any salt thereof, or composition containing the substance or salt of the invention is suitable for administration to a subject or patient.

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates (Polymorphism in Pharmaceutical Solids by K. R. Morris, Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together (Chem Commun, 17; 1889-1896, by O. Almarsson and M. J. Zaworotko, 2004). A general review of multi-component complexes is available in J. Pharm. Sci., 64(8), 1269-1288, by Haleblian (August 1975).

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two-dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The pharmaceutically acceptable salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, a chiral sulfinamide or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in one or both of drug or substrate tissue distribution studies. The radioactive isotopes tritium, i.e., ³H, and carbon-14, i.e., ¹⁴C, are particularly useful for this purpose in view of their ease of incorporation and ready means of detection.

Substitution with deuterium, i.e., ²H, may afford certain therapeutic advantages resulting from greater metabolic stability.

Substitution with positron emitting isotopes, such as ¹¹C, ¹⁸F, ¹⁵O and ¹³N, may be useful in Positron Emission Topography (PET) studies for examining substrate receptor occupancy.

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by C₁-C₁₀ alkanoyl, —COCH₂NH₂ or the amino group is derivatized with an amino acid;
(vi) when a compound of the invention contains a primary or secondary amino functionality (—NH₂ or —NHR where R H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to,
(i) where the compound of the invention contains an alkyl group, a hydroxyalkyl derivative thereof (—CH→—COH):
(ii) where the compound of the invention contains an alkoxy group, a hydroxy derivative thereof (—OR→—OH);
(iii) where the compound of the invention contains a tertiary amino group, a secondary amino derivative thereof (—NRR′→—NHR or —NHR′);
(iv) where the compound of the invention contains a secondary amino group, a primary derivative thereof (—NHR→—NH₂);
(v) where the compound of the invention contains a phenyl moiety, a phenol derivative thereof (-Ph→-PhOH);
(vi) where the compound of the invention contains an amide group, a carboxylic acid derivative thereof (—RCONH₂→RCOOH);
(vii) where the compound of the invention contains an amide group, an amine derivative thereof (—RCONR′→NH2R′); and
(viii) where the compound of the invention contains an alkyl-substituted pyrazole, the dealkylated pyrazole derivative thereof; and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the invention comprises pharmaceutical compositions.

A “pharmaceutical composition” refers to a mixture of one or more of the compounds of the present invention, or a pharmaceutically acceptable salt thereof, as an active ingredient and at least one pharmaceutically acceptable excipient.

The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient.

A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atom/zer (preferably an atom/zer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 1.0 mg to about 500 mg of the active ingredient, or in another embodiment, from about 1.0 mg to about 100 mg of active ingredient. Intravenously, doses may range from about 0.01 to about 10 mg/kg/minute during a constant rate infusion.

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may also be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsion comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsion comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

### Administration and Dosing

The term “treating”, “treat” or “treatment” as used herein embraces both prophylactic and palliative treatment i.e., treatment relieving, alleviating or slowing the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and also encompass mammals in utero. Preferably, said animal is a human. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both);
(3) ameliorating the disease; for example, ameliorating a disease, condition or disorder in an individual that is experiencing or displaying the pathology or symptomatology of the disease, condition or disorder (i.e., reversing the pathology or symptomatology or both); and
(4) clearing out latent viral reservoirs in the body that exist following initial infection and may or may not be contributing to prolonged pathology or symptomatology of the disease, condition or disorder in individuals.

Typically, a compound of the invention is administered in an amount effective to treat a condition as described herein. The compounds of the invention may be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Therapeutic Methods and Uses

The compounds of the invention inhibit the activity of the papain-like protease and may thus be useful in the treatment, prevention, suppression, and amelioration of diseases, disorders and conditions mediated by the papain-like protease, in particular viral infections such as coronaviruses infections.

Examples of such coronavirus infections include, but are not limited to, diseases or conditions in which coronaviruses are implicated like common cold, Middle East respiratory syndrome (MERS), severe acute respiratory syndrome (SARS) or COVID-19 (Coronavirus disease 2019).

### Co-Administration

The compounds of the invention may be used alone, or in combination with one or more other therapeutic agents. The invention provides any of the uses, methods or compositions as defined herein wherein the compound of the invention, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In one embodiment, the compounds of this invention may be administered in combination with other therapeutic agents, which may provide greater clinical benefit. Such additional therapeutic agents include, but are not limited to, vital RNA polymerase inhibitors, Mpro inhibitors, nucleoside inhibitors, host factor inhibitors, other PLpro inhibitors and metabolism boosting agents that leads to reduction in virus replication or host response and may thus contribute to greater clinical benefit. An example of viral RNA polymerase inhibitor is remdesivir. Examples of MPro inhibitors include, but are not limited to, nirmatrelvir (also known as “PF-07321332”), PBI-0451, bofutrelvir (also known as “FB2001”), EDP-235, ensitrelvir (also known as “S-217622”) and ALG-097111.

Additional metabolism boosting agents such as ritonavir may also be used in combination with the compounds of the present invention or with combinations of the compounds of the present invention with other therapeutic agents as indicated above, in order to increase the therapeutic effect.

Examples of greater clinical benefits includes, but are not limited to, a larger reduction in symptoms, a faster time to alleviation of symptoms, reduced lung pathology, a larger reduction in the amount of coronavirus in the patient (viral load), and decreased disease severity or mortality.

These agents and compounds of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent or rapid test. In other embodiments, the kit includes the compound or a pharmaceutical composition thereof, one or more therapeutic agents, such as a viral RNA polymerase inhibitor—e.g., remdesivir-, a MPro inhibitor—e.g., nirmatrelvir, PBI-0451, bofutrelvir, EDP-235, ensitrelvir and ALG-097111—a nucleoside inhibitor, a host factor inhibitor, another PLpro inhibitor or a metabolism boosting agent, and optionally a diagnostic agent or rapid test. In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the compounds of the present invention. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection and deprotection methods is also within the skill in the art. For a general description of protecting groups and their use, see March's Advanced Organic Chemistry: Reactions, Mechanisms, and Structure 8^{th} Edition.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

In the non-limiting Examples and Preparations that illustrate the invention and that are set out in the description, and in the following General Schemes, the following abbreviations, definitions and analytical procedures may be referred to:

### Abbreviations

°2θ is degrees 2-theta;
AcCl is acetyl chloride;
AcOH is acetic acid;
ADH-101 is alcohol dehydrogenase 101;
APCl is atmospheric pressure chemical ionization;
aq is aqueous;
BH₃Me₂S is (dimethyl sulphide)trihydroboron;
BINAP is 1,1′-binaphthalene-2,2′-diyl)bis(diphenylphosphine;
Bn is benzyl;
Boc is tert-butoxycarbonyl;
Boc₂O is di-tert-butyl dicarbonate;
B₂Pin₂ is Bis(pinacolato)diboron;
br is broad;
tBu is tert-butyl;
tBuOH is tert-butanol;
tBuOK is potassium tert-butoxide;
tBuXPhos is 2-di-tert-butylphosphino-2′,4′,6′-triisopropylbiphenyl;
tBuXPhos-Pd Gen-3 is [(2-di-tert-butylphosphino-2′,4′,6′-triisopropyl-1,1′-biphenyl)-2-(2′-amino-1,1′-biphenyl)]palladium(II) methanesulfonate;
° C. is degrees celcius;
CataCXium A is Di(1-adamantyl)-n-butylphosphine;
CBz is benzyloxycarbonyl;
CbzOSu is N-(Benzyloxycarbonyloxy)succinimide;
CDCl₃ is deutero-chloroform;
CDI is 1,1′-carbonyldiimidazole;
δ is chemical shift;
d is doublet;
dd is doublet of doublets;
ddd is doublet of doublet of doublets;
dt is doublet of triplets;
DCE is 1,2-dichloroethane;
DCM is dichloromethane (also known as methylene chloride);
DIAD is diisopropyl azodicarboxylate;
(−)-DIP-Chloride™ is (−)-B-chlorodiisopinocampheylborane;
DIPEA or DIEA is N-ethyldiisopropylamine, also known as N,N-diisopropylethylamine;
DMA is N,N-dimethylacetamide;
DME is 1,2-dimethoxyethane;
DMAP is 4-dimethylaminopyridine;
DMF is N,N-dimethylformamide;
DMSO is dimethyl sulfoxide;
DMSO-d₆ is deuterodimethylsulfoxide;
DPPP is 1,3-bis(diphenylphosphino)propane;
EA is ethyl acetate
EDC is N-(3-dimethylaminopropyl)-N′-ethylcarbodiimide;
EDC.HCl is N-(3-dimethylaminopropyl)-N′-ethylcarbodiimide hydrochloride;
E1 is electron impact ionization;
EtMgBr is ethylmagnesium bromide;
eq. is equivalent
ES is electron scatter;
ESI is electrospray ionization;
Et₂O is diethyl ether;
EtOAc is ethyl acetate;
EtOH is ethanol;
Et₃N is triethylamine;
g is gram;
GCMS is gas chromatography-mass spectrometry;
HATU is 1-[bis(dimethylamino)methylene]-1H-1,2,3-triazolo[4,5-b]pyridinium 3-oxid hexafluorophosphate;
HPLC is high performance liquid chromatography;
HOAc is acetic acid;
HOBt is 1-hydroxybenzotriazole hydrate;
h is hour(s);
IPA is isopropyl alcohol;
iPrOAc is isopropyl acetate;
Ir[dF(CF₃)ppy]₂(dtbpy)PF₆ is [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate;
KOAc is Potassium acetate;
KRED101 is ketoreductase 101 enzyme;
KSAc is potassium thioacetate;
L is liter;
LCMS is liquid chromatography mass spectrometry;
LDA is lithium diisopropylamide;
LG is Leaving Group;
Ir[dF(CF₃)ppy]₂(dtbbpy)PF₆ is [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate;
m is multiplet;
M is molar;
m-CPBA is 3-chloroperbenzoic acid;
Me is methyl;
MeCN is acetonitrile;
MeMgBr is methylmagnesium bromide;
MeNHOMe HCl is N,O-dimethylhydroxylamine hydrochloride;
MeOD_d₄ is deuterated methanol;
MeOH is methanol;
MeI is methyl iodide;
2-MeTHF is 2-methyl tetrahydrofuran;
mg is milligram;
MHz is mega Hertz;
min is minute(s);
mL is milliliter;
mmol is millimole;
mol is mole;
MS (m/z) is mass spectrum peak;
MsCI is mesyl chloride;
Ms₂O is methanesulphonic anhydride;
MTBE is tert-butyl methyl ether;
NaBH(OAc)s is sodium acetoxyborohydride;
NADP+ is nicotinamide adenine dinucleotide phosphate;
NiCl₂·glyme is nickel (II) chloride ethylene glycol dimethyl ether complex;
NMR is nuclear magnetic resonance;
ODS is octadecyl-silica;
ORTEP is Oak Ridge Thermal Ellipsoid Plot;
[PdCl(allyl)]₂ is allylpalladium(II) chloride dimer;
Pd(tBu₃P)₂ is bis(tri-tert-butylphosphine)palladium(0);
Pd/C is palladium on carbon;
Pd₂(dba)₃ is palladium tris(dibenzylideneacetone)dipalladium(0);
Pd(dppf)Cl₂ is [1,1′-bis(diphenylphophino)ferrocene]dichloropalladium(II);
Pd(OAc)₂ is palladium(II) acetate;
Pd(PPh₃)₄ is tetrakis(triphenylphosphine)palladium(0);
PE is Petroleum ether;
Pet. ether is the petroleum fraction consisting of aliphatic hydrocarbons and boiling in the range 35-60° C.;
PG is Protecting Group;
PhI(CF₃CO₂)₂ is [Bis(trifluoroacetoxy)iodo]benzene;
PMB is para-methoxybenzyl;
PMB-NH₂ is para-methoxybenzylamine;
Polycat 5 @ is bis(2-dimethylaminoethyl)(methyl)amine
PPh₃ is triphenylphosphine;
pH is power of hydrogen;
ppm is parts per million;
PSD is position sensitive detector;
psi is pounds per square inch;
PXRD is powder X-ray diffraction;
PyBrOP is Bromotripyrrolidinophosphonium hexafluorophosphate;
q is quartet;
rpm is rotation per minute;
rt is room temperature;
RT is retention time;
Ru-phos is 2-Dicyclohexylphosphino-2′,6′-diisopropoxybiphenyl;
s is singlet;
sat. is saturated;
SEM-Cl is 2-(trimethylsilyl)ethoxymethyl chloride;
SFC is supercritical fluid chromatography;
t is triplet;
T₃P is propylphosphonic anhydride;
TBAF is tert-butyl ammonium fluoride;
TBDMSCI is tert-butyldimethylsilyl chloride;
t-Bu₃P·BF₄ is tri-tert-butylphosphonium tetrafluoroborate;
TEA is Triethylamine;
TFA is trifluoroacetic acid;
THF is tetrahydrofuran;
Ti(O-iPr)₄ is titanium(IV) isopropoxide;
TLC is thin layer chromatography;
TMEDA is N,N,N′N′-tetramethylethylenediamine;
TMSCI is trimethylsilyl chloride;
TMSCN is trimethylsilyl cyanide;
TMSCHN₂ is (diazomethyl)trimethylsilane;
TsCl is p-toluenesulfonyl chloride;
Ts₂O is p-toluenesulfonic anhydride;
UPLC is ultra-performance liquid chromatography;
μL is microliter;
μmol is micromole;
wt is weight
Xantphos is 4,5-bis(diphenylphosphno)-9,9-dimethylxanthene; and
x-phos is 2-dicyclohexylphosphino-2′,4′,6′-triisopropylbiphenyl.

### General Methods:

The following schemes and written descriptions provide general details regarding the preparation of the compounds of the invention.

The compounds of the invention may be prepared by any method known in the art for the preparation of compounds of analogous structure. In particular, the compounds of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of Formula (I), and compounds that fall within Formula (I).

In addition, the skilled person will appreciate that it may be necessary or desirable at any stage in the synthesis of compounds of the invention to protect one or more sensitive groups, to prevent undesirable side reactions. In particular, it may be necessary or desirable to protect amino or alcohol groups. The protecting groups (PGs) used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 2 (“Protection for the Hydroxyl Group, including 1,2- and 1,3-Diols”), incorporated herein by reference, which also describes methods for the removal of such groups.

Some of the compounds of the present invention contain a single chiral center with stereochemical designation (R or S) and others will contain two separate chiral centers with stereochemical designation (R or S). It will be apparent to one skilled in the art that most of the synthetic transformations can be conducted in a similar manner whether the materials are enantioenriched or racemic. Moreover, the resolution to the desired optically active material may take place at any desired point in the sequence using well known methods such as described herein and in the chemistry literature.

Certain processes for the manufacture of the compounds of this invention and intermediates thereof are provided as further features of the invention and are illustrated by the following reaction schemes. Other processes are described in the experimental section. The schemes and examples provided herein (including the corresponding description) are for illustration only, and not intended to limit the scope of the present invention.

Unless stated otherwise, the variables in General Schemes below have the same meanings as defined herein.

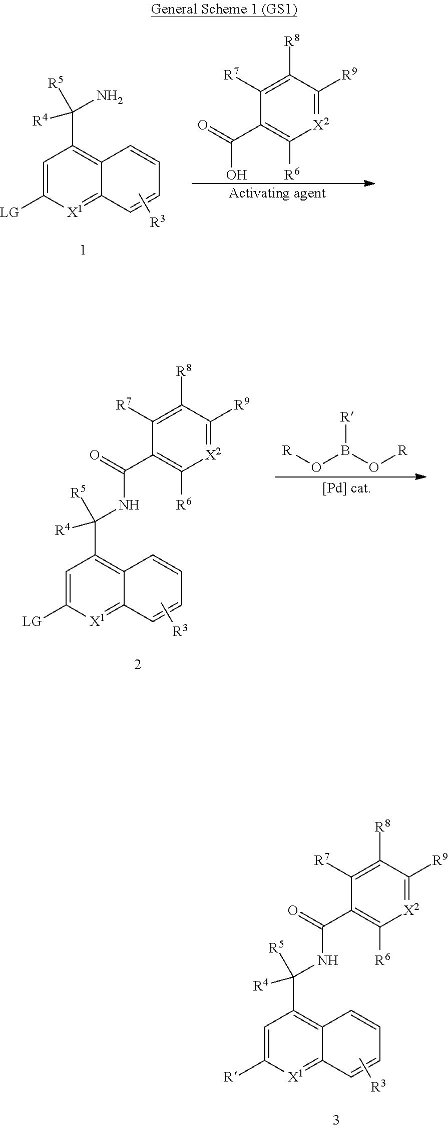

General Scheme 1 describes a general method for preparing compounds of Formula 1, wherein X¹, X², and R³ to R⁹ are as defined in Embodiment 1. An amine starting material 1, either commercially available or prepared using methods standard in the art, may be coupled with a relevant carboxylic acid to provide compound 2. The carboxylic acids of interest are either readily commercially available or synthesizable using straightforward techniques familiar to those skilled in the art. For example, such acids may be prepared using conditions known in WO 2005/102389, or similar publications such as U.S. Pat. No. 4,182,775. This amide formation reaction can be accomplished through standard amide coupling conditions using coupling reagents such as 1,1′-carbonyldiimidazole, 2,4,6-tripropyl-1,3,5,2,4,6-trioxatriphosphinane 2,4,6-trioxide (T3P), 0-(7-azabenzotriazol-1-yl)-N,N,N′,N′-tetramethyluronium hexafluorophosphate (HATU), or others. In certain cases, the carboxylic acid may also contain a protecting group which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Following the formation of compound 2, straightforward Palladium mediated coupling of commercially available boronates R′B(OR)₂, where R is either H or the carbon of a cyclic boronic ester such as the pinacol ester of the boronic acid and R′ is a subset of R¹ in Formula (I), can be used to afford compound 3. More specifically, R′ is a member of the optionally substituted Cyc¹ group as defined in Formula (I). The leaving group (LG) represents one of several leaving groups displaced in Suzuki-type couplings, such as a halogen or triflate group. Additionally, the boronate may also contain a protecting group that can be removed following the Palladium coupling procedure. Procedures commonly known in the art for Suzuki coupling reactions may be found in Name Reactions, A Collection of Detailed Reaction Mechanisms and Synthetic Applications, Jie Jack Li, 2021 or Applied Organic Chemistry: Reaction Mechanisms and Experimental Procedures in Medicinal Chemistry by Surya K. De, 2021.

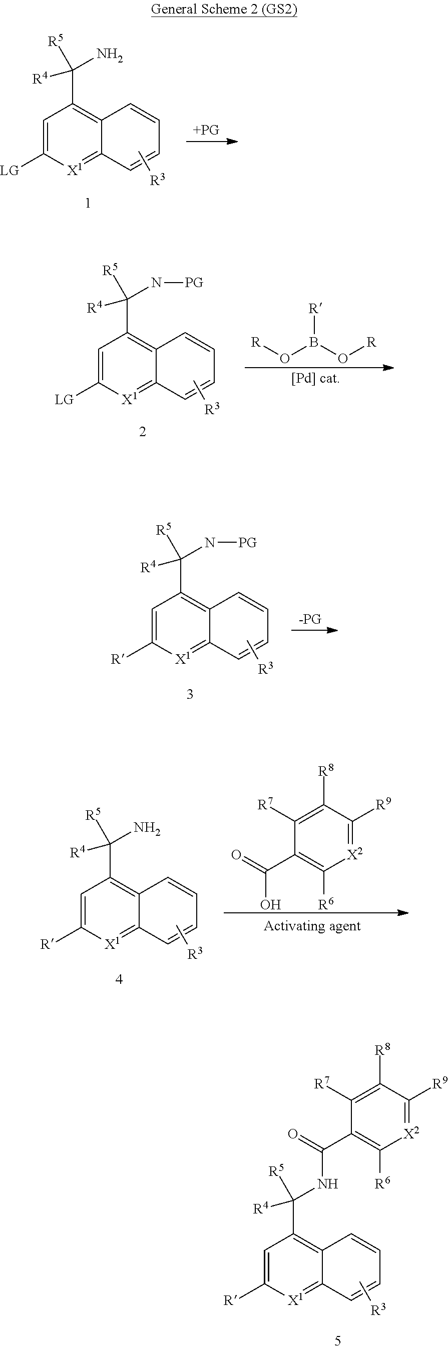

General Scheme 2 describes an alternative general method for preparing compound 5 of Formula 1, wherein X¹, X² and R³ to R⁹ are as defined in Embodiment 1. An amine starting material 1, either commercially available or prepared using methods standard in the art, may be first optionally protected using a protecting group (PG), where PG is selected from amine protecting groups including, but not limited to, those enumerated in Protecting Groups, 10 Georg Thieme Verlag, 1994. Following the optional protection step, compound 2 is coupled with commercially available boronate R′B(OR)₂, which is as defined in GS1, to afford product 3. The leaving group LG represents one of several leaving groups commonly used in Suzuki-type couplings, such as a halogen or triflate group. Straightforward deprotection using methods commonly employed in the art yields compound 4, which is then subjected to an amide formation reaction with an appropriate acid to afford compound 5. The carboxylic acids of interest are either readily purchasable or synthesizable using straightforward techniques in the art. For example, such acids may be prepared using conditions known in WO 2005/102389, or similar publications such as U.S. Pat. No. 4,182,775. The amide coupling reaction itself can be accomplished through standard amide formation conditions using coupling reagents such as 1,1′-carbonyldiimidazole, 2,4,6-tripropyl-1,3,5,2,4,6-trioxatriphosphinane 2,4,6-trioxide (T3P), 0-(7-azabenzotriazol-1-yl)-N,N,N′,N′-tetramethyluronium hexafluorophosphate (HATU), or others. In certain cases, substituents R³ to R⁹ may also contain a protecting group which may be appended or removed by additional steps in the synthetic sequence using conditions known in the art (March's Advanced Organic Chemistry: Reactions, Mechanisms, and Structure 8^{th} Edition or Protecting Groups, 10 Georg Thieme Verlag, 1994). In some instances, the addition and removal of PG shown above, may prove unnecessary, and thus these steps may be excised from the scheme above in order to access compound 5.

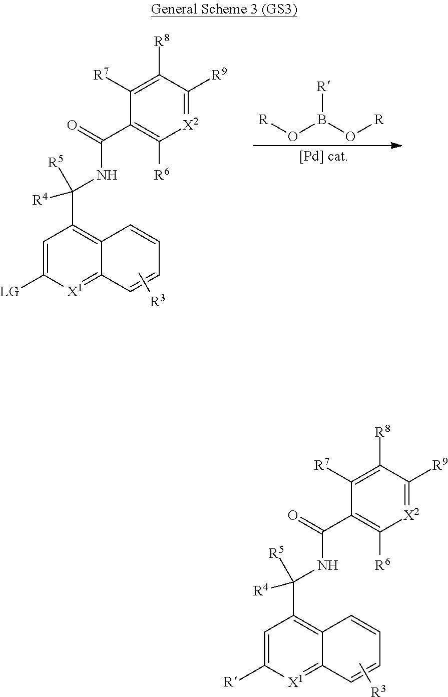

GS3 provides a general preparation for the synthesis of a subset of Formula 1, where R′B(OR)₂ is as defined in GS1. In GS3, the starting material is an amide with an activated leaving group LG appropriate for coupling, that may be prepared using GS1, step 1, or other straightforward methods. X¹, X² and R³ to R⁹ are all as defined in Embodiment 1 of Formula (I). It should be understood to those skilled in the art that the boronate building block R′B(OR)₂, representing a boronic acid or ester, may also contain a protecting group that may be appended or removed following the Palladium coupling procedure. In certain cases, X¹, X² and R³ to R⁹ may also contain a protecting group which may be appended or removed by additional steps in the synthetic sequence using conditions known in the art (March's Advanced Organic Chemistry: Reactions, Mechanisms, and Structure 8^{th} Edition or Protecting Groups, 10 Georg Thieme Verlag, 1994). The leaving group LG represents one of several leaving groups displaced in Suzuki-type couplings, such as a halogen or triflate group. Procedures commonly known in the art for Suzuki coupling reactions may be found in Name Reactions, A Collection of Detailed Reaction Mechanisms and Synthetic Applications, Jie Jack Li, 2021 or Applied Organic Chemistry: Reaction Mechanisms and Experimental Procedures in Medicinal Chemistry by Surya K. De, 2021.

A more specific embodiment of GS3, shown below, describes a general method for the synthesis of quinolines with aryl or heteroaryl substitutions in the 2-position, such as the product, which is a compound of Formula (I).

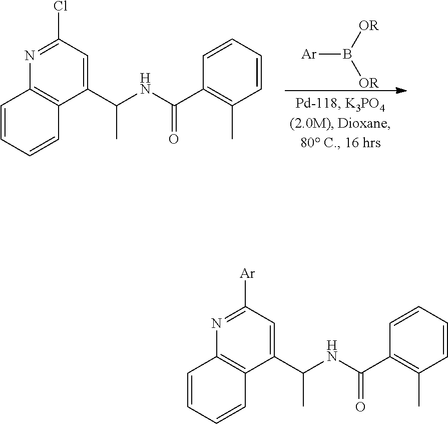

In the above scheme, Ar represents an optionally substituted aryl or heteroaryl group as defined in Embodiment 1. Compounds of this form may be prepared using coupling with the appropriate boronates and a Palladium catalyst under Suzuki-like conditions. In particular, compounds of this specific form may be prepared in a parallel medicinal chemistry format using the following procedure:
1. Dispense template N-(1-(2-chloroquinolin-4-yl)ethyl)-2-methylbenzamide (60 μmol, 1.0 eq.) and the desired boronic acid/ester Ar—B(OR)(OR), where R is as defined in GS1, (90 μmol, 1.5 eq.) to 8 mL reaction vials.
2. Dispense 450 μL Dioxane to each vial.
3. Dispense Pd-118 (6.0 μmol, 0.1 eq.) to each vial under N₂.
4. Dispense 150 μL K₃PO₄ (2.0 M, 300 μmol, 5.0 eq.) to each vial.
5. Cap vials and shake at 80° C. for 16 hours.
6. Spot check reactions by LC-MS.
7. Remove solvent by Speedvac.
8. Wash the mixture with 1.0 mL of H₂O and extract with EtOAc (2.0 mL×3).
9. Collect organic layer and evaporate solvent by Speedvac.
10. Purify residue by preparative HPLC to give desired product.

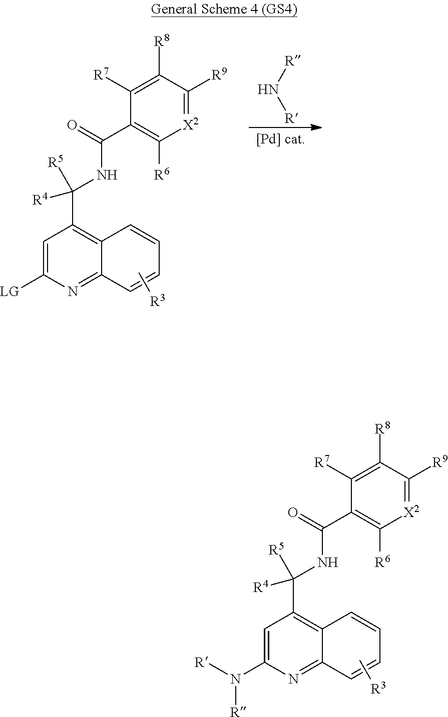

GS4 describes a general method for the synthesis of 2-aminoquinolines derivatives according to Formula (I). Compounds of the general form may be prepared under Buchwald-like conditions using a Pd catalyst. X² and R³ to R⁹ are all as defined in Embodiment 1. It should be understood to those skilled in the art that the amino building block NH(R′)R″ represents a primary or secondary amine of R¹ in Formula (I) that may also contain a protecting group that can be appended or removed following the Palladium coupling procedure. In particular, N(R′)R″ is of the specific forms —N(R^{a})R^{b}, —N(R^{a})(C₁-C₆ alkoxy-C₁-C₆ alkyl), —N(R^{a})(C₁-C₆ alkyl-C(═O)—C, —C₆ alkyl), —N(R^{a})(C₁-C₆ alkyl-C(═O)—N(R^{c})R^{d}), —N(R^{a})(C₁-C₆ alkyl-SO₂—N(R^{c})R^{d}), or -L¹-L²-L³-Cyc¹ of Formula (I) that contain a primary or secondary amine. In certain cases, X² and R³ to R⁹ may also contain a protecting group which may be appended or removed by additional steps in the synthetic sequence using conditions known in the art (March's Advanced Organic Chemistry: Reactions, Mechanisms, and Structure 8^{th} Edition or Protecting Groups, 10 Georg Thieme Verlag, 1994). The leaving group LG represents one of several leaving groups displaced in Buchwald-type couplings, such as a halogen or triflate group. Procedures commonly known in the art for Buchwald-like coupling reactions may be found in Name Reactions, A Collection of Detailed Reaction Mechanisms and Synthetic Applications, Jie Jack Li, 2021 or Applied Organic Chemistry: Reaction Mechanisms and Experimental Procedures in Medicinal Chemistry by Surya K. De, 2021.

A more specific embodiment of GS4, shown below, describes a method for the synthesis of quinolines with amino substitutions in the 2-position from a 2-chloroquinoline starting material

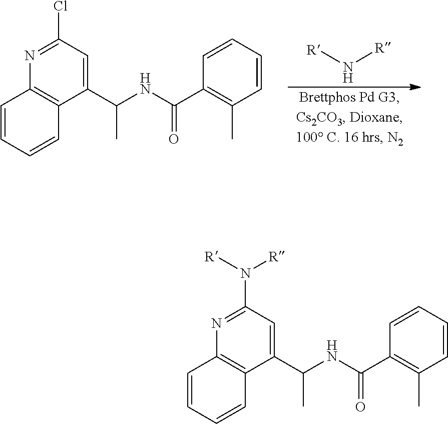

Compounds of this specific embodiment of GS4 can be prepared in a parallel medicinal chemistry format under Buchwald-like conditions according to the following procedure:
1. Dispense the template N-(1-(2-chloroquinolin-4-yl)ethyl)-2-methylbenzamide (100 μmol, 1.0 eq.) and the amine monomer NH(R′)R″ (150 μmol, 1.5 eq.) to 8 mL reaction vials.
2. Dispense 1000 ul Dioxane to each vial.
3. Dispense Brettphos Pd G3 (10.0 μmol, 0.1 eq.) to each vial under N₂.
4. Dispense Cs₂CO₃ (300.0 μmol, 3.0 eq.) to each vial.
5. Cap vials and shake at 100° C. for 16 hours.
6. Spot check reactions by LC-MS.
7. Remove solvent by Speedvac.
8. Wash the mixture with 1.0 mL of H₂O and extract with EtOAc (2.0 mL×3).
9. Purify residue by preparative HPLC to give desired product.

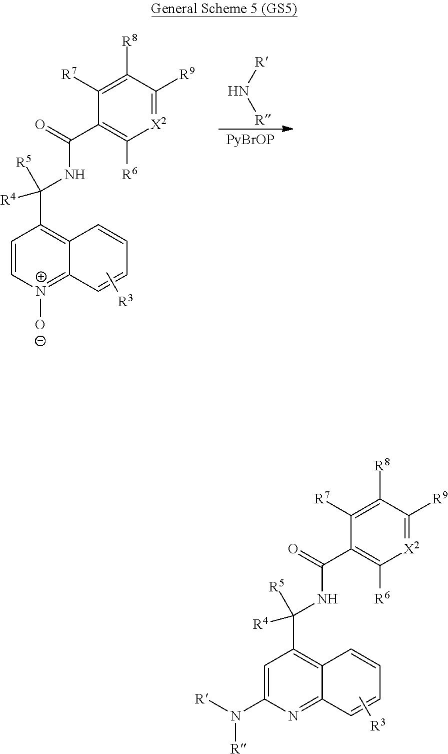

GS5 describes a general method for the synthesis of 2-aminoquinoline derivatives of Formula (I) such as the shown product from the appropriate N-oxide starting material. X² and R³ to R⁹ are all as defined in Embodiment 1. It should be understood to those skilled in the art that the amino building block NH(R′)R″ is as previously defined in GS4 and represents a primary or secondary amine that may also contain a protecting group that may be appended or removed following the formation of the aminoquinoline. In certain cases, X² and R³ to R⁹ may also contain a protecting group which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Procedures commonly known in the art for aminoquinoline formation from N-oxide starting materials are reviewed in Org. Lett. 2006, 8, 9, 1929-1932.

A specific embodiment of GS5, shown below, describes a method for the synthesis of quinolines with amino substitutions in the 2-position from the N-oxide starting material 4-(1-(2-methylbenzamido)ethyl)quinoline 1-oxide.

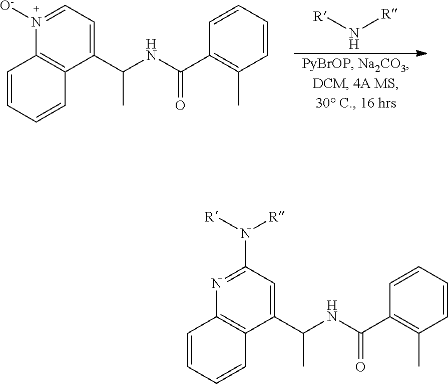

In practice, these compounds can be prepared in a parallel format using the following conditions:
1. Dispense 4-(1-(2-methylbenzarnido)ethyl)quinoline 1-oxide (100 μmol, 1.0 eq.) and the respective NH(R′)R″ amine monomer (150 μmol, 1.5 eq.) to 8 mL reaction vials.
2. Dispense 500 ul dichloromethane to each vial.
3. Dispense Bromo-tris-pyrrolidino-phosphonium hexafluorophosphate (PyBrOP) (120 μmol, 1.2 eq.) to each vial.
4. Dispense Na₂CO₃ (300.0 μmol, 3.0 eq) to each vial.
5. Dispense 4 A Molecular Sieves (30 mg) to each vial.
6. Cap vials and shake at 30° C. for 16 hours.
7. Spot check reactions by LC-MS.
8. Filter and concentrate filtrate by Speedvac.
9. Purify residue by preparative HPLC to give desired product.

### General Scheme 6 (GS6)

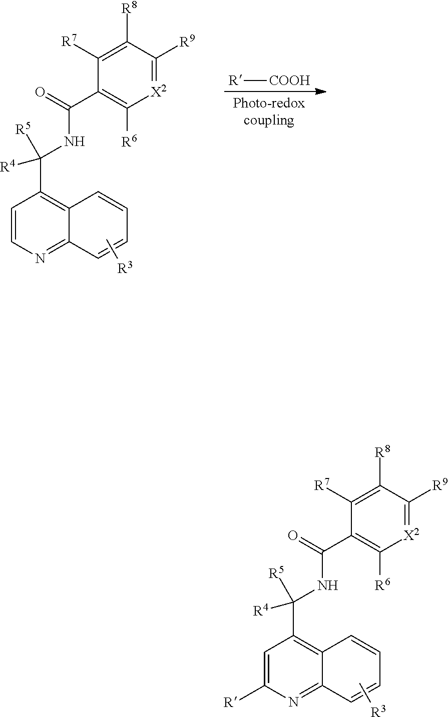

GS6 describes a general method for the synthesis of quinolines of Formula 1, with an aliphatic carbon linked substitution in the 2-position. X² and R³ to R⁹ are all as defined in Embodiment 1. It should be understood to those skilled in the art that the carboxylic acid building block R′—COOH, where R′ contains an aliphatic carbon linker to the acid, can also contain a protecting group that may be appended or removed following the formation of the aminoquinoline. In particular, R′ is a member of -L¹-L²-L³-Cyc¹, C₁-C₆ alkyl, C₁-C₆ aminoalkyl, C₁-C₆ cyanoalkyl, C₁-C₆ alkyl-C(═O)—N(R^{a})R^{b} as defined in Formula (1). In certain cases, X² and R³ to R⁹ may also contain a protecting group which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994).

A first specific embodiment of GS6, shown below, describes a method for the synthesis of these compounds in a parallel format using Minisci-like photoredox conditions.

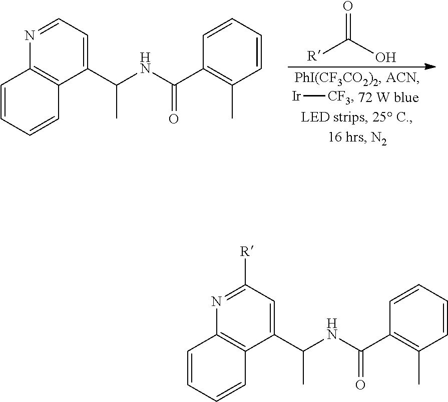

In particular, the following describes the synthesis of quinolines using a template quinoline and carboxylic acids of the form R′COOH, where R represents alkyl and heteroalkyl groups as defined in R¹ of Embodiment 1, using the parallel medicinal chemistry method described below:
1. Dispense (R)-2-methyl-N-(1-(quinolin-4-yl)ethyl)benzamide (100 μmol, 1.0 eq.) to 8 mL reaction vials in a glove box.
2. Dispense the carboxylic acid monomer R′COOH (80 μmol, 8.0 eq.) to each vial.
3. Dispense PhI(CF₃CO₂)₂(200 μmol, 2.0 eq.) and Ir[dF(CF₃)ppy]₂(dtbbpy)PF₆ (1 μmol, 0.01 eq.) to each vial.
4. Dispense 1000 ul of MeCN to each vial.
5. Cap vials and transfer out of the glove box.
6. Stir the mixture at 25° C. for 16 hours under 72 W blue LED strip.
7. Check the reactions by LC-MS.
8. Filter and purify the solution by preparative HPLC to give desired product.

As an alternative second specific embodiment of GS6, the following photoredox conditions may also be used to produce compounds of Formula 1, where R′ represents optionally substituted alkyl and heteroalkyl groups as defined in R, of Embodiment 1.

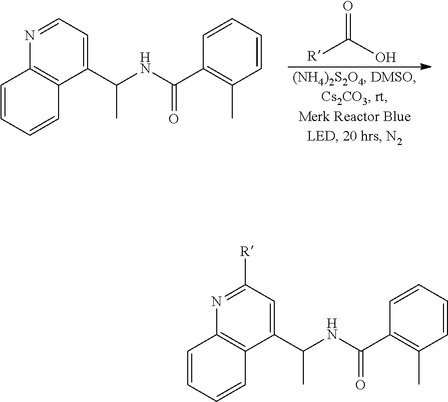

In particular, the procedure may be carried out in a parallel format, using the specific procedure below:
1. Dispense (R)-2-methyl-N-(1-(quinolin-4-yl)ethyl)benzamide (100 μmol, 1.0 eq.) to 8 mL in glove box.
2. Dispense carboxylic acid monomer R′COOH (80 μmol, 8.0 eq.) to each vial.
3. Dispense Cs₂CO₃ (200 μmol, 2.0 eq.)
4. Dispense (NH₄)₂S₂O₈(200 μmol, 2.0 eq.) to each vial.
5. Dispense 1000 ul of DMSO to each vial.
6. Cap and transfer out of the glove box.
7. Stir the mixture at room temperature for 20 hours under Integrated photoreactor-Royal Blue (450 nm) LED lights, with fan rate: 4700 rpm, stir rate: 1000 rpm, LED light intensity: 100%.
8. Check the reaction by LC-MS.
9. Filter and purify the solution by preparative HPLC to give desired product.

In certain instances, where the product of the above procedure includes a protective group, additional steps can be taken to remove the protective group. In specific instances, the tert-Butyloxycarbonyl (Boc) amino protecting group can be removed using the following procedure:
1. Dispense the Boc protected starting material (about 100 μmol, 1.0 eq.) to 8-mL vial.
2. Dispense 1000 ul of dichloromethane and 200 of TFA to the vial.
3. Cap and shake at 30° C. for 16 hours.
4. Remove the solvent by Speedvac.
5. Purify residue by preparative HPLC to give the final product.

For all of the above general schemes, as will be clear to those familiar in the art, an extra separation step can also be added in order to isolate the desired stereochemical form. Additionally, that chiral form can be employed for any subsequent steps if the enantiopure product is desired rather than the racemic form shown in the schemes. If the final product of such schemes is racemic, the mixture of enantiomers or diastereomers formed may be separated using supercritical fluid or reversed-phase chromatography with a chiral column.

### EXAMPLES

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

Reactions were performed in air or, when oxygen- or moisture-sensitive reagents or intermediates were employed, under an inert atmosphere (nitrogen or argon). When appropriate, reaction apparatuses were dried under dynamic vacuum using a heat gun, and anhydrous solvents (Sure-Seal™ products from Aldrich Chemical Company, Milwaukee, Wisconsin or DriSolv™ products from EMD Chemicals, Gibbstown, NJ) were employed. In some cases, commercial solvents were passed through columns packed with 4 Å molecular sieves, until the following QC standards for water were attained: a) <100 ppm for dichloromethane, toluene, N,N-dimethylformamide and tetrahydrofuran; b) <180 ppm for methanol, ethanol, 1,4-dioxane and diisopropylamine. For very sensitive reactions, solvents were further treated with metallic sodium, calcium hydride, or molecular sieves, and distilled just prior to use. Other commercial solvents and reagents were used without further purification. For syntheses referencing procedures in other Examples or Methods, reaction conditions (reaction time and temperature) may vary. Products were generally dried under vacuum before being carried on to further reactions or submitted for biological testing.

When indicated, reactions were heated by microwave irradiation using Biotage Initiator or Personal Chemistry Emrys Optim/zer microwaves. Reaction progress was monitored using thin-layer chromatography (TLC), liquid chromatography-mass spectrometry (LCMS), high-performance liquid chromatography (HPLC), and/or gas chromatography-mass spectrometry (GCMS) analyses. TLC was performed on pre-coated silica gel plates with a fluorescence indicator (254 nm excitation wavelength) and visualized under UV light and/or with iodine, potassium permanganate, cobalt(II) chloride, phosphomolybdic acid, and/or ceric ammonium molybdate stains. LCMS data were acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluent was analyzed using a Waters ZQ mass spectrometer scanning in both positive and negative ion modes from 100 to 1200 Da. Other similar instruments were also used. HPLC data were generally acquired on an Agilent 1100 Series instrument, using the columns indicated, acetonitrile/water gradients, and either trifluoroacetic acid or ammonium hydroxide modifiers. GCMS data were acquired using a Hewlett Packard 6890 oven with an HP 6890 injector, HP-1 column (12 m×0.2 mm×0.33 μm), and helium carrier gas. The sample was analyzed on an HP 5973 mass selective detector scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were performed by chiral supercritical fluid chromatography (SFC), generally using Berger or Thar instruments; columns such as ChiralPAK-AD, -AS, -IC, Chiralcel-OD, or -OJ columns; and CO₂ mixtures with methanol, ethanol, 2-propanol, or acetonitrile, alone or modified using trifluoroacetic acid or propan-2-amine. UV detection was used to trigger fraction collection. For syntheses referencing procedures in other Examples or Methods, purifications may vary: in general, solvents and the solvent ratios used for eluents/gradients were chosen to provide appropriate Retention factors (Rfs) or retention times.

Mass spectrometry data are reported from LCMS analyses. Mass spectrometry (MS) was performed via atmospheric pressure chemical ionization (APCl), electrospray Ionization (ESI), electron impact ionization (EI) or electron scatter (ES) ionization sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, δ) referenced to the deuterated solvent residual peaks (chloroform, 7.26 ppm; CD2HOD, 3.31 ppm; acetonitrile-d2, 1.94 ppm; dimethyl sulfoxide-d5, 2.50 ppm; DHO, 4.79 ppm). The peak shapes are described as follows: s, singlet; d, doublet; t, triplet; q, quartet; quin, quintet; m, multiplet; br s, broad singlet; app, apparent. Analytical SFC data were generally acquired on a Berger analytical instrument as described above. Optical rotation data were acquired on a PerkinElmer model 343 polarimeter using a 1 dm cell. Microanalyses were performed by Quantitative Technologies Inc. and were within 0.4% of the calculated values.

Unless otherwise noted, chemical reactions were performed at room temperature (about 23 degrees Celsius).

Unless noted otherwise, all reactants were obtained commercially and used without further purification, or were prepared using methods known in the literature.

The terms “concentrated”, “evaporated”, and “concentrated in vacuo” refer to the removal of solvent at reduced pressure on a rotary evaporator with a bath temperature less than 60° C. The term “room temperature or ambient temperature” means a temperature between 18 to 25° C.

Hydrogenation may be performed in a Parr shaker under pressurized hydrogen gas, or in Thales-nano H-Cube flow hydrogenation apparatus at full hydrogen and a flow rate between 1-2 mL/min at specified temperature.

HPLC, UPLC, LCMS, GCMS, and SFC retention times were measured using the methods noted in the procedures.

In some examples, chiral separations were carried out to separate enantiomers or diastereomers of certain compounds of the invention (in some examples, the separated enantiomers are designated as ENT-1 and ENT-2, according to their order of elution; similarly, separated diastereomers are designated as DIAST-1 and DIAST-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds are indicated either by the absence of drawn or described stereochemistry, or by the presence of (+/−) adjacent to the structure; in this latter case, the indicated stereochemistry represents just one of the two enantiomers that make up the racemic mixture.

The compounds and intermediates described below were named using the naming convention provided with ACD/ChemSketch 2017.2.1, File Version C40H41, Build 99535 (Advanced Chemistry Development, Inc., Toronto, Ontario, Canada). The naming convention provided with ACD/ChemSketch 2017.2.1 is well known by those skilled in the art and it is believed that the naming convention provided with ACD/ChemSketch 2017.2.1 generally comports with the IUPAC (International Union for Pure and Applied Chemistry) recommendations on Nomenclature of Organic Chemistry and the CAS Index rules.

The ¹H NMR spectra of many of the compounds herein indicate a mixture of rotamers, due to the presence of amide and/or carbamate functionality and have been tabulated to reflect the presence of more than one rotamer.

### Preparations

The following describe preparations of some starting materials or intermediates used for preparation of certain compounds of the invention.

### Preparation P1: (R)-2-methyl-N-(1-(quinolin-4-yl)ethyl)benzamide

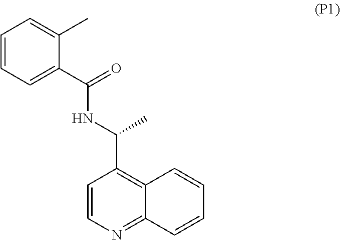 [SMILES: Cc1ccccc1C(=O)N[C@H](C)c1ccnc2ccccc12]

### Step 1. Preparation of (R)-1-(quinolin-4-yl)ethan-1-amine

Racemic 1-(quinolin-4-yl)ethan-1-amine and pure (R)-1-(quinolin-4-yl)ethan-1-amine are prepared as described in WO 2008/007211.

### Step 2. Preparation of (R)-2-methyl-N-(1-(quinolin-4-yl)ethyl)benzamide

To a solution of (R)-1-(quinolin-4-yl)ethan-1-amine (2.8 g, 16.26 mmol) in DMF (28 mL) were added 2-methylbenzoic acid (2.66 g, 19.51 mmol), HOBt (7.4 g, 19.51 mmol) and DIPEA (4.2 g, 32.52 mmol) at 20° C. The mixture was cooled to 0° C. before adding EDCI (3.74 g, 19.51 mmol). The reaction was then stirred at 20° C. for 5 hrs. TLC (PE/EA=1/1.5) showed that that starting material was consumed and new spots were detected. Water (200 mL) was then added to the reaction, and the mixture was extracted with ethyl acetate (6×100 mL). The organic layers were combined, washed with brine, filtered and concentrated under reduced pressure to give a residue, which was purified by column chromatography on a silica gel column (petroleum ether/ethyl acetate=100/0 to 1/1) to give (R)-2-methyl-N-(1-(quinolin-4-yl)ethyl)benzamide (3 g, 64%) as white solid. ¹H NMR (400 MHz, CDCl₃) δ 1.73 (d, J=6.75 Hz, 3H), 2.41 (s, 3H), 6.01-6.12 (m, 1H), 6.27 (br d, J=7.88 Hz, 1H), 7.11-7.24 (m, 2H), 7.27 (s, 2H), 7.40 (d, J=4.50 Hz, 1H), 7.62 (ddd, J=8.32, 7.00, 1.19 Hz, 1H), 7.74 (td, J=7.63, 1.25 Hz, 1H), 8.13 (d, J=8.50 Hz, 1H), 8.22 (d, J=8.38 Hz, 1H), 8.85 (d, J=4.38 Hz, 1H).

### Preparation P2: (R)-4-(1-(2-methylbenzamido)ethyl)quinoline 1-oxide

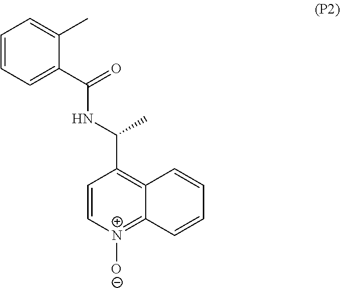 [SMILES: Cc1ccccc1C(=O)N[C@H](C)c1cc[n+]([O-])c2ccccc12]

To a solution of (R)-2-methyl-N-(1-(quinolin-4-yl)ethyl)benzamide (2.5 g, 8.61 mmol) in dichloromethane (25 mL) was added m-CPBA (2.62 g, 12.92 mmol) portion-wise with stirring at 0° C. The reaction was warmed to 25° C. and stirred at 25° C. for 12 h. TLC (petroleum ether/tetrahydrofuran=1/1) showed that starting material was consumed and a new spot was detected. The reaction was quenched with sat. aq. NaHCO₃ (100 mL). The organic layer was washed with brine, dried over Na₂SO₄, filtered and concentrated under reduced pressure to give a residue, which was purified by column chromatography on a silica gel column (eluting with petroleum ether/tetrahydrofuran=100/1 to 0/1) to give (R)-4-(1-(2-methylbenzamido)ethyl) quinoline 1-oxide (2.5 g, 94.8%) as a white solid. ¹H NMR (400 MHz, CDCl₃) δ ppm 1.77 (br d, J=6.75 Hz, 3H), 2.47 (s, 3H), 5.88-6.14 (m, 1H), 7.09 (br d, J=6.13 Hz, 1H), 7.17-7.25 (m, 2H), 7.28-7.34 (m, 1H), 7.39 (br d, J=7.38 Hz, 1H), 7.58 (br dd, J=11.88, 8.13 Hz, 2H), 8.18 (br d, J=8.25 Hz, 1H), 8.28 (br d, J=8.88 Hz, 2H).

### Preparation P3: (R)—N-(1-(2-chloroquinolin-4-yl)ethyl)-2-methylbenzamide

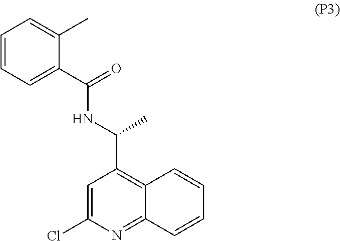 [SMILES: Cc1ccccc1C(=O)N[C@H](C)c1cc(Cl)nc2ccccc12]

To a solution of (R)-4-(1-(2-methylbenzamido)ethyl)quinoline 1-oxide (1.5 g, 4.9 mmol) and DIPEA (6.33 g, 48.96 mmol) in dichloromethane (50 mL) was added POCl₃ (2.8 g, 24.48 mmol) dropwise with stirring at 0° C. The reaction was warmed to 25° C. and stirred at 25° C. for 12 h. TLC (THF) showed that starting material was consumed and new spots were detected. The reaction was transferred to a cooled sat. aq. NaHCO₃ solution (100 mL) and extracted with dichloromethane (3×50 mL). The combined organic layer was washed with brine, dried over Na₂SO₄, filtered and concentrated under reduced pressure to give a residue, which was purified by column chromatography on a silica gel column (eluting with petroleum ether/tetrahydrofuran=100/1 to 3/1) to give (R)—N-(1-(2-chloroquinolin-4-yl)ethyl)-2-methylbenzamide (0.6 g, 37.73%) as a white solid. ¹H NMR (400 MHz, CDCl₃) δ1.70-1.85 (m, 3H), 2.45 (s, 3H), 5.95-6.17 (m, 2H), 7.18-7.26 (m, 2H), 7.31-7.37 (m, 2H), 7.42 (s, 1H), 7.60-7.70 (m, 1H), 7.74-7.84 (m, 1H), 8.07 (d, J=8.00 Hz, 1H), 8.20 (d, J=8.50 Hz, 1H). LCMS (ESI): m/z=325.1 [M+1]⁺.

### Preparation P4: 1-(3-bromonaphthalen-1-yl)ethan-1-amine

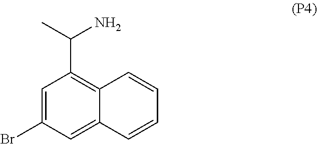

### Step 1. Preparation of (E)-1-(3-bromonaphthalen-1-yl)ethan-1-one oxime

To a solution of 1-(3-bromonaphthalen-1-yl)ethan-1-one (4400 mg, 17.66 mmol) in MeOH (90 mL) was added hydroxylamine hydrochloride (3680 mg, 53.0 mmol) at 30° C. The mixture was adjusted to pH 6-6.5 using aq. NaOH (40%) then stirred at 50° C. for 16 h. TLC (PE/EA=2011, UV) showed starting material was consumed and two new spots were observed. The reaction mixture was then concentrated in vacuo to give (E)-1-(3-bromonaphthalen-1-yl)ethan-1-one oxime (4600 mg, 98.6%) as a white solid which was used in step 2 directly.

### Step 2. Preparation of 1-(3-bromonaphthalen-1-yl)ethan-1-amine

To a solution of (E)-1-(3-bromonaphthalen-1-yl)ethan-1-one oxime (4850 mg, 18.36 mmol) in AcOH (100 mL) and MeOH (20 mL) was added Zn (5820 mg, 88.98 mmol) at 30° C. Then the mixture was stirred at 80° C. for 16 h. The reaction mixture was then concentrated in vacuo to remove solvents, diluted with ethyl acetate (200 mL), basified with sat. aq. NaHCO₃ (200 mL), then filtered. The filtrate was extracted with ethyl acetate (100 mL×2). The combined organic layer was concentrated in vacuo to give 1-(3-bromonaphthalen-1-yl)ethan-1-amine (4.5 g, 98.0%) as a brown gum, which was used in subsequent steps directly. LCMS (ESI): m/z=234.9 [M+H]⁺.

### Preparation P5: 1-(3-(1-methyl-1H-pyrazol-4-yl)naphthalen-1-yl)ethan-1-amine

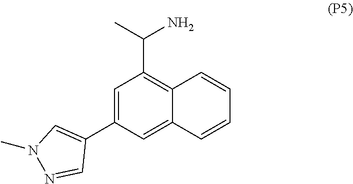 [SMILES: CC(N)c1cc(-c2cnn(C)c2)cc2ccccc12]

### Step 1. Preparation of tert-butyl(1-(3-bromonaphthalen-1-yl)ethyl)carbamate

To a solution of 1-(3-bromonaphthalen-1-yl)ethan-1-amine (4500 mg, 17.99 mmol) in dichloromethane (90.0 mL) were added TEA (3640 mg, 36.0 mmol) and Boc₂O (3930 mg, 18.0 mmol) at 25° C. The mixture was stirred at 25° C. for 16 h. TLC (EA, UV) showed that the starting material was consumed and one main spot was observed. The reaction mixture was concentrated in vacuo to give a crude, which was purified by flash chromatography (40 g silica gel column, ethyl acetate in PE from 0 to 10%) to give tert-butyl(1-(3-bromonaphthalen-1-yl)ethyl)carbamate (4.5 g, 71.4%) as a yellow solid. ¹H NMR (400 MHz, CDCl₃) δ=8.08 (br d, J=8.0 Hz, 1H), 7.92 (d, J=1.5 Hz, 1H), 7.84-7.67 (m, 1H), 7.63-7.42 (m, 3H), 7.63-7.42 (m, 1H), 5.57 (br s, 1H), 4.88 (br s, 1H), 1.58 (s, 3H), 1.44 (br s, 9H).

### Step 2. Preparation of tert-butyl(1-(3-(1-methyl-1H-pyrazol-4-yl)naphthalen-1-yl)ethyl) carbamate

To a solution of tert-butyl(1-(3-bromonaphthalen-1-yl)ethyl)carbamate (500.0 mg, 1.43 mmol) in 1,4-dioxane (10.0 mL) and H₂O (2.0 mL) were added 1-methyl-4-(4,4,5,5-tetramethyl-1,3,2-dioxaborolan-2-yl)-1H-pyrazole (297 mg, 1.43 mmol), K₃PO₄ (909 mg, 4.28 mmol) and Pd(dppf)Cl₂ (104 mg, 0.143 mmol). Then the mixture was stirred at 80° C. under N₂ for 16 h, followed by solvent removal and purification by silica gel column chromatography (eluting with ethyl acetate in PE from 0 to 50%) to give tert-butyl(1-(3-(1-methyl-1H-pyrazol-4-yl)naphthalen-1-yl)ethyl)carbamate (460 mg, 91.7%) as a brown oil. LCMS (ESI): m/z=352.1 [M+H]⁺.

### Step 3. Preparation of 1-(3-(1-methyl-1H-pyrazol-4-yl)naphthalen-1-yl)ethan-1-amine

To a solution of tert-butyl(1-(3-(1-methyl-1H-pyrazol-4-yl)naphthalen-1-yl)ethyl)carbamate (460.0 mg, 1.31 mmol) in dichloromethane (6.0 mL) was added HCl (4M, 4.0 mL, in dioxane) at 25° C. After addition, the solution was stirred at room temperature (25° C.) for 2 h then concentrated in vacuo to give a residue. The residue was partitioned between H₂O (10 mL) and ethyl acetate (3 mL×2). The layers were separated, and the aqueous phase was adjusted to pH=9-10 with NH₃·H₂O and further extracted with ethyl acetate (3 mL×2), the organic extracts were combined and concentrated under vacuum to give 1-(3-(1-methyl-1H-pyrazol-4-yl)naphthalen-1-yl)ethan-1-amine (300 mg, 91.2%) as a brown oil, which was used in next steps directly.

### Preparation 11: 1-(3-(1H-pyrazol-5-yl)naphthalen-1-yl)ethan-1-amine

P11 was prepared in a similar manner to the preparation of P5 in Step 2-3, using tert-butyl(1-(3-bromonaphthalen-1-yl)ethyl)carbamate and an appropriate boronic acid or boronic ester.

| | | Analogous | |
|---|---|---|---|
| Prep. | Structure; | method, | Characterizing |
| # | Compound Name | reactants | Data |
| P11 | 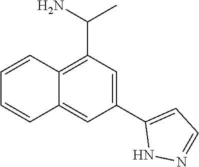 [SMILES: CC(N)c1cc(-c2ccn[nH]2)cc2ccccc12] 1-(3-(1H-pyrazol-5-yl)naphthalen- 1-yl)ethan-1-amine | Preparation P5, Step 2, 1- (tetrahydro-2H- pyran-2-yl)-5- (4,4,5,5- tetramethyl- 1,3,2- dioxaborolan- 2-yl)-1H- pyrazole; then Preparation P5, Step 3 | LCMS (ESI): m/z = 220.9, [M + H − NH₃]⁺, 239.0 [M + H− NH₃ + H₂O]⁺ |

### Preparation P6: (R)-5-cyano-2-methyl-N-(1-(quinolin-4-yl)ethyl)benzamide

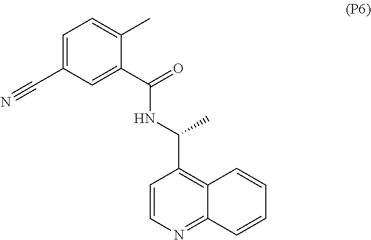 [SMILES: Cc1ccc(C#N)cc1C(=O)N[C@H](C)c1ccnc2ccccc12]

To a mixture of (R)-1-(quinolin-4-yl)ethan-1-amine (0.6 g, 3.48 mmol) in DMF (6 mL) were added HOBt (564.89 mg, 4.18 mmol), 5-cyano-2-methylbenzoic acid (648.99 mg, 4.18 mmol) and DIEA (900.51 mg, 6.97 mmol). Then to above the solution was added EDCI (648.99 mg, 4.18 mmol) at 0° C., the mixture was stirred at 25° C. for 12 h. LCMS showed that starting material was consumed and a product with desired mass was detected. The mixture was diluted with water (10 mL) and extracted with ethyl acetate (3×15 mL). The combined organic layer was dried over Na₂SO₄, filtered and concentrated under reduced pressure to give a residue, which was purified by chromatography on a silica gel column (PE:EA=100/1 to 2/1) to yield (R)-5-cyano-2-methyl-N-(1-(quinolin-4-yl)ethyl)benzamide (1.1 g, 62.53%) as a yellow solid. ¹H NMR (400 MHz, CDCl₃) δ1.79 (d, J=6.40 Hz, 3H), 2.49 (s, 3H), 6.08-6.20 (m, 2H), 7.27 (s, 3H), 7.34 (d, J=7.91 Hz, 1H), 7.44 (d, J=4.52 Hz, 1H), 7.55-7.62 (m, 2H), 7.63-7.69 (m, 1H), 7.73 (t, J=7.47 Hz, 1H), 3.19 (m, 2H), 3.92 (d, J=4.52 Hz, 1H). LCMS (ESI): m/z 316.0 [M+H]⁺

### Preparation P7: tert-butyl(3-((1-(2-chloroquinolin-4-yl)ethyl)carbamoyl)-4-methylbenzyl) carbamate

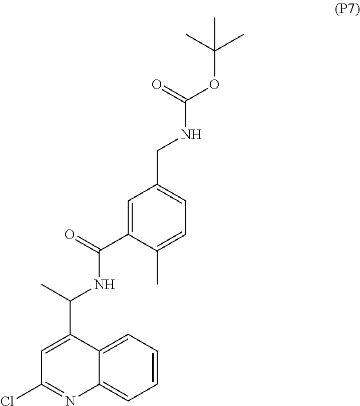 [SMILES: Cc1ccc(CNC(=O)OC(C)(C)C)cc1C(=O)NC(C)c1cc(Cl)nc2ccccc12]

### Step 1. Preparation of 4-(1-(5-cyano-2-methylbenzamido)ethyl)quinoline 1-oxide

To a solution of 5-cyano-2-methyl-N-(1-(quinolin-4-yl)ethyl)benzamide (P6) (5600 mg, 17.76 mmol) in dichloromethane (200 mL) was added m-CPBA (7210 mg, 35.5 mmol). The reaction mixture was stirred at 25° C. for 16 h. LCMS showed that the starting material was consumed and a desired mass was found. The mixture was quenched with a saturated aqueous solution of Na₂S₂O₃ until the KI test paper was colorless and adjusted to pH>8 using saturated aqueous solution of Na₂CO₃. The mixture was then stirred for an additional 0.5 h before being extracted with dichloromethane (200 mL×2). The combined organic layer was dried over Na₂SO₄, filtered, and concentrated under reduced pressure to give a residue, which was purified on a silica column (PE:EA=50:50 to 0:100, then EA:MEOH=10:1, UV) to give 4-(1-(5-cyano-2-methylbenzamido)ethyl)quinoline 1-oxide (5.2 g, 33.4%) as a yellow solid. ¹H NMR (400 MHz, DMSO-d₅) δ ppm 1.53 (d, J=6.35 Hz, 3H), 2.36 (s, 3H), 5.74-5.87 (m, 1H), 7.50 (dd, J=15.59, 7.15 Hz, 2H), 7.74-7.94 (m, 4H), 3.37 (d, J=7.95 Hz, 1H), 8.58-8.68 (m, 2H), 9.16 (d, J=7.46 Hz, 1H). LCMS (ESI): m/z=332.1 [M+H]⁺.

### Step 2. Preparation of N-(1-(2-chloroquinolin-4-yl)ethyl)-5-cyano-2-methylbenzamide

To the solution of 4-(1-(5-cyano-2-methylbenzamido)ethyl)quinoline 1-oxide (5200 mg, 15.69 mmol) in 1,2-dichloroethane (200 mL) was added POCl₃ (7220 mg, 47.1 mmol). The reaction was stirred at 60° C. for 16 h. LCMS showed that the starting material was consumed and the major peak was of the desired product. The reaction was then concentrated to a yellow oil and adjusted to pH=7 with aq. NaHCO₃ at 0° C. before being extracted with dichloromethane (250 mL×2). The organic layers were separated, dried over Na₂SO₄, filtered and concentrated to give a crude product as a yellow solid. The yellow solid was suspended in dichloromethane (50 mL)/THF (50 mL) and stirred at 30° C. for 10 min. The solid was collected by filtration to give the N-(1-(2-chloroquinolin-4-yl)ethyl)-5-cyano-2-methylbenzamide (2.2 g, 40.1%) as a yellow solid. The filtrate was purified by a flash silica gel column (PE:THF=100:0 to 50:50) to give an additional amount of N-(1-(2-chloroquinolin-4-yl)ethyl)-5-cyano-2-methylbenzamide (2.0 g, 36.4%) as a yellow solid. ¹H NMR (400 MHz, DMSO-dc) 6 ppm 1.59 (d, J=6.97 Hz, 3H), 2.36 (s, 3H), 5.87 (quin, J=6.94 Hz, 1H), 7.49 (d, J=7.95 Hz, 1H), 7.55-7.64 (m, 1H), 7.59 (s, 1H), 7.75 (td, J=7.67, 1.28 Hz, 1H), 7.81-7.93 (m, 3H), 8.02 (dd, J=8.44, 0.86 Hz, 1H), 8.35 (d, J=7.95 Hz, 1H), 9.20 (d, J=7.46 Hz, 1H). LCMS (ESI): m/z=350.1 [M+H]⁺.

### Step 3. Preparation of tert-butyl(3-((1-(2-chloroquinolin-4-yl)ethyl)carbamoyl)-4-methylbenzyl)carbamate

To the solution of N-(1-(2-chloroquinolin-4-yl)ethyl)-5-cyano-2-methylbenzamide (2200 mg, 6.289 mmol) in MeOH (120.0 mL) was added (Boc)₂O (374 mg 1.72 mmol) and NiCl₂ (815 mg, 6.29 mmol). Then NaBH₄ (1300 mg, 34.36 mmol) was added dropwise to the mixture at 0° C. The reaction was stirred at 30° C. for 1 h. LCMS showed that most of the starting material was consumed and the desired product was exposed. The reaction was quenched with sat. aq. NH₄Cl (60 mL) at 0° C. and extracted with ethyl acetate (100 mL×2). The organic layers were separated, dried over Na₂SO₄, filtered, concentrated, and purified by flash silica gel chromatography (PE:EA/dichloromethane (1/1)=100:0 to 50:50) to give the tert-butyl(3-((1-(2-chloroquinolin-4-yl)ethyl)carbamoyl)-4-methylbenzyl)carbamate (1.4 g, 49.0%) as a white solid. ¹H NMR (400 MHz, DMSO-dc) 6 ppm 1.39 (s, 9H), 1.57 (d, J=6.97 Hz, 3H), 2.25 (s, 3H), 4.12 (br d, J=6.11 Hz, 2H), 5.80-5.96 (m, 1H), 7.20 (s, 2H), 7.22 (s, 1H), 7.40 (br t, J=6.11 Hz, 1H), 7.57 (s, 1H), 7.70-7.80 (m, 1H), 7.82-7.92 (m, 1H), 8.01 (d, J=7.70 Hz, 1H), 8.37 (d, J=8.19 Hz, 1H), 9.03 (br d, J=7.46 Hz, 1H). LCMS (ESI): m/z=398.1 [M+H−tBu]⁺.

### Preparation P8: 5-(((tert-butoxycarbonyl)amino)methyl)-2-methylbenzoic acid

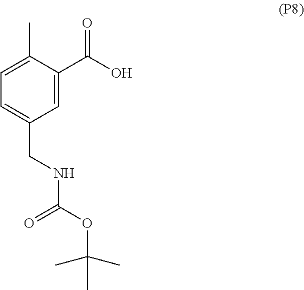 [SMILES: Cc1ccc(CNC(=O)OC(C)(C)C)cc1C(=O)O]

### Step 1. Preparation of methyl 5-(((tert-butoxycarbonyl)amino)methyl)-2-methylbenzoate

To a solution of methyl 5-cyano-2-methylbenzoate (300.0 mg, 1.71 mmol) in CH₃OH (10 mL) were added (Boc)₂O (448 mg, 2.05 mmol), NaBH₄ (140 mg 3.70 mmol), and NiCl₃ (444 mg, 3.42 mmol) at 0° C. The mixture was kept at 0° C. for 30 min then at 25° C. for 12 h. LCMS showed the desired product was produced. TLC (PE/EA=10/1, UV) showed that a new spot was exposed. The reaction was then quenched by sat. aq. NH₄Cl (5 mL). The mixture was extracted with ethyl acetate (30 mL×3). The combined organic layer was dried by Na₂SO₄, filtered, and concentrated to give a residue, which was purified by flash silica gel chromatography (PE:EA=100:0 to 90:10) to give methyl 5-(((tert-butoxycarbonyl)amino)methyl)-2-methylbenzoate (300 mg, 62.7%) as a colorless oil. ¹H NMR (400 MHz, CDCl₃, 300 K) δ (ppm)=7.87-7.80 (s, 1H), 7.39-7.29 (d, 1H), 7.25-7.20 (d, 1H), 4.97-4.77 (s, 1H), 4.39-4.25 (d, 2H), 3.92-3.88 (s, 3H), 2.61-2.58 (s, 3H), 1.48 (s, 9H).

### Step 2. Preparation of 5-(((tert-butoxycarbonyl)amino)methyl)-2-methylbenzoic acid

To a solution of methyl 5-(((tert-butoxycarbonyl)amino)methyl)-2-methylbenzoate (2500.0 mg, 8.950 mmol) in THF (18.0 mL) and MeOH (12.0 mL) and H₂O (6.0 mL) was added LiOH·H₂O (1500 mg, 35.8 mmol). The reaction was stirred at 25° C. for 12 h. LCMS showed the starting material was consumed and a desired mass was found. MeOH was removed in vacuo and the aqueous residue was acidified to pH 6 with 2N HCl. The precipitate was filtered and dried in vacuo to give 5-(((tert-butoxycarbonyl)amino)methyl)-2-methylbenzoic acid (2300 mg, 96.9%) as a white solid. ¹H NMR (400 MHz, MeOD-d₄, 297 K) δ (ppm)=7.85 (s, 1H), 7.37-7.32 (m, 1H), 7.27-7.20 (m, 1H), 4.28-4.21 (m, 2H), 2.58-2.56 (m, 1H), 2.56 (s, 3H), 1.47 (s, 9H). LCMS (ESI): m/z=288.1 [M+Na]⁺.

### Preparation P9: 5-(((tert-butoxycarbonyl)(methyl)amino)methyl)-2-methylbenzoic acid

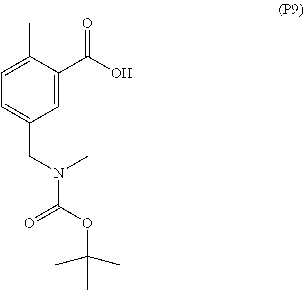 [SMILES: Cc1ccc(CN(C)C(=O)OC(C)(C)C)cc1C(=O)O]

### Step 1. Preparation of methyl 5-(((tert-butoxycarbonyl)(methyl)amino)methyl)-2-methylbenzoate

To a solution of methyl 5-(((tert-butoxycarbonyl)amino)methyl)-2-methylbenzoate (500 mg, 1.79 mmol) in THF (12 mL) was added tBuOK (301 mg, 2.68 mmol) at 0° C. The mixture was stirred at 0° C. for 0.5 h, then a solution of MeI (381 mg, 2.68 mmol) in THF (3.0 mL) was added dropwise at 0° C. The mixture was stirred at 25° C. for an additional 0.5 h. TLC (PE/EA=10/1, UV) showed that the starting material was consumed and one main spot was observed. The reaction mixture was poured into ice water (20 mL) then extracted with ethyl acetate (20 mL×2). The combined organic layer was dried with Na₂SO₄ and filtered. The filtrate was concentrated in vacuo to give methyl 5-(((tert-butoxycarbonyl)(methyl)amino)methyl)-2-methylbenzoate (450 mg, 85.7%) as a yellow gum which was used in the next step directly. ¹H NMR (400 MHz, CDCl₃) δ=7.68-7.58 (m, 1H), 7.17-7.01 (m, 3H), 4.33-4.14 (m, 2H), 373 (s, 2H), 2.68 (br s, 2H), 2.43 (s, 3H), 1.44 (br s, 2H), 1.37-1.22 (in, 9H).

### Step 2. Preparation of 5-(((tert-butoxycarbonyl)(methyl)amino)methyl)-2-methylbenzoic acid

To a solution of methyl 5-(((tert-butoxycarbonyl)(methyl)amino)methyl)-2-methylbenzoate (450 mg, 1.53 mmol) in THF (6.0 mL) and H₂O (2.0 mL) was added NaOH (184 mg, 4.60 mmol) at 25° C. The mixture was stirred at 25° C. for 40 h. TLC (PE/EA=10/1, UV) showed that most of the starting material remained. Additional NaOH (184 mg, 4.60 mmol) was added to the reaction at 25° C., and the mixture was stirred at 50° C. for 16 h. TLC (PE/EA=10/1, UV) showed some of the starting material remained and one new spot on the baseline was observed. The reaction mixture was stirred at 70° C. for another 5 h. TLC (PE/EA=10/1, UV) showed that the starting material was consumed. The reaction mixture was then concentrated in vacuo, diluted in H₂O (10 mL) and extracted with ethyl acetate (20 mL×2), the organic layer was discarded. The aqueous phase was adjusted to pH=4-5 using aq. HCl (1N), then the solution was extracted with ethyl acetate (20 mL×3), the combined organic layer was dried with Na₂SO₄, filtered, and the filtrate was concentrated in vacuo to give 5-(((tert-butoxycarbonyl)(methyl)amino)methyl)-2-methylbenzoic acid (390 mg, 91.0%) as a yellow gum. ¹H NMR (400 MHz, MeOD-d4) δ=7.73 (br s, 1H), 7.25-7.12 (m, 2H), 4.33 (s, 2H), 2.74 (br s, 3H), 2.47 (s, 3H), 1.38 (br s, 9H).

### Preparation P10: benzyl(R)-(1-(2-chloroquinolin-4-yl)ethyl)carbamate

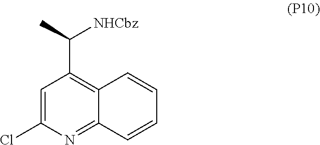 [SMILES: C[C@@H](NC(=O)OCc1ccccc1)c1cc(Cl)nc2ccccc12]

### Step 1. Preparation of benzyl(R) (1-(quinolin-4-yl)ethyl)carbamate

To a stirred solution of (R)-1-(quinolin-4-yl)ethan-1-amine (8400.0 mg, 48.77 mmol) in dichloromethane (500 mL) were added Et₃N (7400 mg, 73.2 mmol) and CbzOSu (13400 mg, 53.7 mmol) at 0° C. The reaction mixture was stirred at 25° C. for 2 h. LCMS showed a mass peak of the desired product. The reaction was evaporated in vacuo and then purified by silica gel chromatography via Biotage (120 g silicon column, PE:EA=1:0 to 0:1) to afford benzyl(R)-(1-(quinolin-4-yl)ethyl)carbamate (14600 mg, 97.7%) as a yellow oil. LCMS (ESI): m/z=307 [M+H]⁺.

### Step 2. Preparation of (R)-4-(1-(((benzyloxy)carbonyl)amino)ethyl)quinoline 1-oxide

To a solution of benzyl(R)-(1-(quinolin-4-yl)ethyl)carbamate (7000.0 mg, 22.85 mmol) in dichloromethane (100 mL) was slowly added m-CPBA (6960 mg, 34.3 mmol) in several portions at 5° C. After that, the ice-water bath was removed, and the mixture was warmed up to 30° C. and stirred for 20 h. LCMS showed a mass peak of the desired product. The reaction was evaporated in vacuo and purified by silica gel chromatography via Biotage (120 g silica column, PE:EA=1:0 to 0:1) to afford (R)-4-(1-(((benzyloxy)carbonyl)amino)ethyl)quinoline 1-oxide (5700 mg, 77.4%) as a yellow oil. LCMS (ESI): m/z=323 [M+H]⁺.

### Step 3. Preparation of benzyl(R)-(1-(2-chloroquinolin-4-v)ethyl)carbamate

To the solution of (R)-4-(1-(((benzyloxy)carbonyl)amino)ethyl)quinoline 1-oxide (5700.0 mg, 17.68 m mol) in dichloromethane (20.0 mL) was added POCl₃ (27100 mg, 177 mmol) at 0° C. After that, the mixture was stirred at 25° C. for 16 h. LCMS showed a mass peak of the desired product. NaHCO₃ (80 mL sat. aq.) was then added to the reaction mixture at 25° C. The aqueous phase was extracted with dichloromethane (20 mL×2). The combined organic layer was dried with anhydrous Na₂SO₄, filtered, evaporated in vacuo, and purified by silica gel chromatography via Biotage (120 g silicon column, PE:EA=1:0 to 0:1) to afford the product benzyl(R)-(1-(2-chloroquinolin-4-yl)ethyl)carbamate (4960 mg, 82.3%) as a yellow solid. ¹H NMR (400 MHz, DMSO-d₆) δ=8.28 (d, J=8.4 Hz, 1H), 8.22 (br d, J=7.6 Hz, 1H), 7.99 (d, J=8.3 Hz, 1H), 7.85 (t, J=7.3 Hz, 1H), 7.71 (br t, J=7.5 Hz, 1H), 7.50 (s, 1H), 7.43-7.19 (m, 5H), 5.76 (s, 2H), 5.56-5.45 (m, 1H), 1.47 (d, J=7.0 Hz, 3H). LCMS (ESI): m/z=340.9 [M+H]⁺.

### Preparation P12: N-(1-(3-bromonaphthalen-1-yl)ethyl)-2-methylpropane-2-sulfinamide

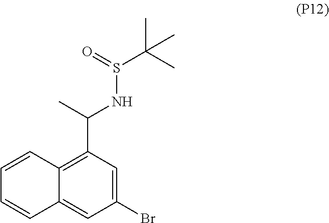 [SMILES: CC(NS(=O)C(C)(C)C)c1cc(Br)cc2ccccc12]

P12 may be prepared from 3-bromonaphthalene-1-carboxaldehyde analogously to the preparation of (R)-2-methyl-N—((S)-1-(3-(trifluoromethoxy)phenyl)ethyl)propane-2-sulfinamide as described in WO 2019/161877.

### Preparation P13: (R)-5-(1-(tert-butoxycarbonyl)piperidine-2-carboxamido)-2-methylbenzoic acid

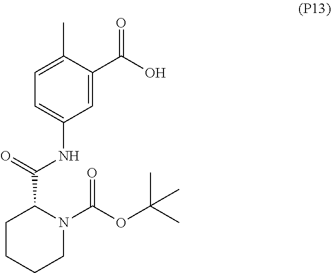 [SMILES: Cc1ccc(NC(=O)[C@H]2CCCCN2C(=O)OC(C)(C)C)cc1C(=O)O]

### Step 1. Preparation of tert-butyl(R)-2-((3-(methoxycarbonyl)-4-methylphenyl)carbamoyl) piperidine-1-carboxylate

To a solution of (2R)-1-[(2-methylpropan-2-yl)oxycarbonyl]piperidine-2-carboxylic acid (1000 mg, 4.37 mmol) in DMF (15 mL) were added methyl 5-amino-2-methylbenzoate (720 mg, 4.36 mmol), HATU (1990 mg, 5.24 mmol), and DIPEA (1690 mg, 13.1 mmol). The reaction was stirred at 25° C. for 12 hours. LCMS showed that a desired mass was produced, and TLC (PE/EA=5/1) showed a new spot was exposed. The mixture was treated with 100 mL H₂O then extracted by EA (20 mL×3). The organic phase was dried by Na₂SO₄, filtered, concentrated, and purified by flash silica gel chromatography (PE:EA=100:0 to 80:20) to give tert-butyl(R)-2-((3-(methoxycarbonyl)-4-methylphenyl)carbamoyl)piperidine-1-carboxylate (1.4 g, 85.3%) as a colourless oil. ¹H NMR (400 MHz, CDCl₃, 300 K) δ (ppm)=8.01 (d, J=2.2 Hz, 1H), 7.68-7.58 (d, 1H), 7.26-7.16 (d, 1H), 5.32 (s, 1H), 4.88 (br s, 1H), 4.18-4.02 (s, 1H), 3.93-3.82 (s, 3H), 2.94-2.79 (m, 1H), 2.56 (s, 3H), 2.42-2.31 (m, 1H), 1.75-1.60 (m, 4H), 1.54 (s, 9H). LCMS (ESI): m/z=399.2 [M+Na]⁺.

### Step 2. Preparation of (R)-5-(1-(tert-butoxycarbonyl)piperidine-2-carboxamido)-2-methylbenzoic acid

To a solution of tert-butyl(R)-2-((3-(methoxycarbonyl)-4-methylphenyl)carbamoyl)piperidine-1-carboxylate (1400.0 mg, 3.719 mmol) in THF (6.0 mL), MeOH (4.0 mL) and H₂O (2.0 mL) was added LiOH·H₂O (624 mg, 14.9 mmol). The reaction was stirred at 25° C. for 12 hours. LCMS showed the starting material was consumed, and a desired mass was found. MeOH was removed in vacuo, and the aqueous residue was acidified to pH=6 with 2N HCl. The precipitate was filtered and dried in vacuo to give (R)-5-(1-(tert-butoxycarbonyl)piperidine-2-carboxamido)-2-methylbenzoic acid (1100 mg, 81.6%) as a white solid. LCMS (ESI): m/z=385.1 [M+Na]⁺.

### Preparation P14: 1-(2-(1-methyl-1H-pyrazol-4-yl)quinolin-4-yl)cyclopropan-1-amine

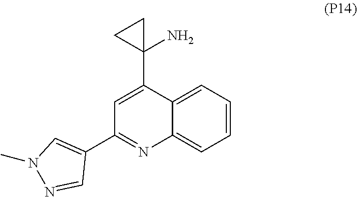 [SMILES: Cn1cc(-c2cc(C3(N)CC3)c3ccccc3n2)cn1]

### Step 1. Preparation of methyl 2-(2-chloroquinolin-4-yl)acetate

To a solution of 2-chloro-4-methylquinoline (1000.0 mg, 6.984 mmol) in THF (30 mL) under N₂ was added LDA (6.0 mL, 2 M, 12 mmol) at −78° C. The reaction mixture was stirred at −78° C. under N₂ for 30 min, followed by the addition of dimethyl carbonate (1270 mg, 14.1 mmol) at 0° C. The reaction mixture was stirred at 0° C. under N₂ for 1 h, then quenched with 3 mL sat. NH₄Cl at 0° C. The reaction mixture was partitioned between ethyl acetate and H₂O (200/50 mL), the organic layer was separated, and the aqueous layer was re-extracted with ethyl acetate (50 mL). The combined organic layer was washed with brine, dried with anhydrous Na₂SO₄, and evaporated in vacuo. The residue was purified by silica gel chromatography using a Biotage (20 g silica gel column, PE:EA=1:0 to 0:1) to afford methyl 2-(2-chloroquinolin-4-yl)acetate (744 mg, 56.1%) as a yellow oil. LCMS (ESI): m/z=236.1, [M+H]⁺.

### Step 2. Preparation of methyl 1-(2-chloroquinolin-4-yl)cyclopropane-1-carboxylate

To a solution of methyl 2-(2-chloroquinolin-4-yl)acetate (3000.0 mg, 12.73 mmol) in DMF (60.0 mL) were added 1,2-dibromoethane (3590 mg, 19.1 mmol), Cs₂CO₃ (8300 mg, 25.5 mmol), tetra-n-butylammonium fluoride (13 mL, 1 M, 13 mmol), and NaOH (1020 mg, 25.5 mmol) at 20° C. The reaction mixture was stirred at 20° C. for 15 h. LCMS showed a mass peak of the desired product. The reaction mixture was then partitioned between ethyl acetate and H₂O (250/100 mL). The organic layer was separated, and the aqueous layer was re-extracted with ethyl acetate (150 mL). The combined organic layer was washed with brine, dried with anhydrous Na₂SO₄, and evaporated in vacuo. The residue was then purified by silica gel chromatography using a Biotage (80 g silica gel column, PE:EA=1:0 to 0:1) to afford methyl 1-(2-chloroquinolin-4-yl)cyclopropane-1-carboxylate (2885 mg, 86.6%) as a yellow solid. LCMS (ESI): m/z=262.0, [M+H]⁺.

### Step 3. Preparation of 1-(2-chloroquinolin-4-yl)cyclopropane-1-carboxylic acid

To a solution of methyl 1-(2-chloroquinolin-4-yl)cyclopropane-1-carboxylate (2885.0 mg, 11.02 mmol) in MeOH (60 mL) was added 10% aq. NaOH (15 mL) at 20° C. The reaction mixture was stirred at 20° C. for 15 h. LCMS showed a mass peak of the desired product. The reaction mixture was then acidified with 1N HCl to pH=1 at 0° C. and partitioned between ethyl acetate and H₂O (250/50 mL). The organic layer was separated, and the aqueous layer was re-extracted with ethyl acetate (150 mL). The combined organic layer was washed with brine, dried with anhydrous Na₂SO₄, and evaporated in vacuo to afford 1-(2-chloroquinolin-4-yl)cyclopropane-1-carboxylic acid (2730 mg, 100%) as a yellow solid. LCMS (ESI): m/z=248.1, [M+H]⁺.

### Step 4. Preparation of 1-(2-chloroquinolin-4-yl)cyclopropan-1-amine

To a solution of 1-(2-chloroquinolin-4-yl)cyclopropane-1-carboxylic acid (2500.0 mg, 10.09 mmol) in toluene (60.0 mL) under N₂, was added diphenylphosphoryl azide (3060 mg, 11.1 mmol) and TEA (2250 mg, 22.2 mmol) at 20° C. The reaction mixture was stirred at 90° C. under N₂ for 2 h, then evaporated in vacuo to afford a crude. The crude was dissolved in 1,4-dioxane (40 mL), followed by adding aq. NaOH (10%, 20 mL) at 20° C. under N₂. The reaction mixture was stirred at 90° C. for 15 h. LCMS showed a mass peak of the desired product. The reaction mixture was then partitioned between ethyl acetate and H₂O (250/150 mL). The organic layer was separated, and the aqueous layer was re-extracted with ethyl acetate (50 mL). The combined organic layer was washed with brine, dried with anhydrous Na₂SO₄, and evaporated in vacuo. The residue was dissolved in 30 mL MeOH, then 10 mL 1 N HCl was added to the solution at 20° C. The reaction mixture was stirred at 20° C. for 1 h, then partitioned between ethyl acetate and H₂O (250/150 mL). The organic layer was discarded, and the aqueous layer was basified with NH₃·H₂O to pH=9 at 0° C., followed by extraction with DCM (200 mL). The organic layer was washed with brine, dried with anhydrous Na₂SO₄ and evaporated in vacuo to afford 1-(2-chloroquinolin-4-yl)cyclopropan-1-amine (1444 mg, 65.4%) as a white solid. LCMS (ESI): m/z=219.1, [M+H]⁺.

### Step 5. Preparation of 1-(2-(1-methyl-1H-pyrazol-4-yl)quinolin-4-yl)cyclopropan-1-amine

To a solution of 1-(2-chloroquinolin-4-yl)cyclopropan-1-amine (1000.0 mg, 4.573 mmol) in 1,4-dioxane (40.0 mL) under N₂ were added aq. Na₂CO₃ (8.0 mL), Pd(dppf)Cl₂ (335 mg, 0.457 mmol), and 1-methyl-4-(4,4,5,5-tetramethyl-1,3,2-dioxaborolan-2-yl)-1H-pyrazole (1900 mg, 9.15 mmol) at 20° C. The reaction mixture was stirred at 100° C. under N₂ for 15 h. LCMS showed a mass peak of the desired product. The reaction mixture was then filtered, and the filtrate was evaporated in vacuo and purified by silica gel chromatography using a Biotage (20 g silica gel column, PE:EA=1:0 to 0:1) to afford 1-(2-(1-methyl-1H-pyrazol-4-yl)quinolin-4-yl)cyclopropan-1-amine (1055 mg, 87.3%) as a yellow oil. LCMS (ESI): m/z=265.1, [M+H]⁺.

### Preparation P15: 1-(4-methoxy-3-(1-methyl-1H-pyrazol-4-yl)naphthalen-1-yl)ethan-1-amine

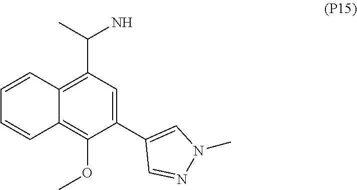 [SMILES: COc1c(-c2cnn(C)c2)cc(C(C)N)c2ccccc12]

### Step 1. Preparation of 3-bromo-4-methoxy-1-naphthaldehyde

3-bromo-4-methoxy-1-naphthaldehyde was synthesized following a similar procedure described in Limaye et al. “Convenient Synthesis of 2-Allyl-3-Bromo-1,4-Dimethoxynaphthalene: Key Intermediate as Building Block for Bioactive Pyranonaphthoquinones”, Synthetic Communications, 42: 313-319, 2012. Specifically, NBS (7170 mg, 40.3 mmol) was added to a solution of 4-methoxy-1-naphthaldehyde (5000.0 mg, 26.85 mmol) in HOAc (100.0 mL) at 20° C. The reaction mixture was stirred at 80° C. for 10 h, at which time LCMS showed a mass peak of the desired product. The reaction mixture was partitioned between ethyl acetate and H₂O (150/50 mL). The organic layer was separated, and the aqueous layer was re-extracted with ethyl acetate (50 ml). The combined organic layer was washed with brine, dried with anhydrous Na₂SO₄, and evaporated in vacuo. The residue was then purified by silica gel chromatography using a Biotage (20 g silica gel column, PE:EA=1:0 to 0:1) to afford 3-bromo-4-methoxy-1-naphthaldehyde (6343 mg, 89.1% yield) as a yellow solid. LCMS (ESI): m/z=265.0, [M+H]⁺.

### Step 2. Preparation of (E)-N-((3-bromo-4-methoxynaphthalen-1-yl)methylene)-2-methylpropane-2-sulfinamide

To a solution of 3-bromo-4-methoxy-1-naphthaldehyde (3000.0 mg, 11.32 mmol) in CH₂Cl₂ (100.0 mL) were added 2-methylpropane-2-sulfinamide (4110 mg, 33.9 mmol) and Cs₂CO₃ (5530 mg, 17.0 mmol) at 20° C. The reaction mixture was stirred at 20° C. for 15 h, at which time LCMS showed a mass peak of the desired product. The reaction mixture was filtered, and the filtrate was evaporated in vacuo. The residue was then purified by silica gel chromatography using a Biotage (80 g silica gel column, PE:EA=1:0 to 0:1) to afford (E)-N-((3-bromo-4-methoxynaphthalen-1-yl)methylene)-2-methylpropane-2-sulfinamide (3505 mg, 84.1% yield) as a yellow oil. LCMS (ESI): m/z=369.9, [M+H]⁺.

### Step 3. Preparation of N-(1-(3-bromo-4-methoxynaphthalen-1-yl)ethyl)-2-methylpropane-2-sulfinamide

To a solution of (E)-N-((3-bromo-4-methoxynaphthalen-1-yl)methylene)-2-methylpropane-2-sulfinamide (3500.0 mg, 9.503 mmol) in THF (70 mL) under N₂ was added MeMgBr (9 mL, 3 M, 27 mmol) at 0° C. The reaction mixture was stirred under N₂ at 20° C. for 2 h, at which time LCMS showed a mass peak of the desired product. The reaction mixture was then quenched with 6 mL sat. NH₄Cl at 0° C. and partitioned between ethyl acetate and H₂O (100/50 mL). The organic layer was separated, and the aqueous layer was re-extracted with ethyl acetate (50 mL). The combined organic layer was washed with brine, dried with anhydrous Na₂SO₄, and evaporated in vacuo to afford N-(1-(3-bromo-4-methoxynaphthalen-1-yl)ethyl)-2-methylpropane-2-sulfinamide (3650 mg, 99.9%) as a yellow oil. LCMS (ESI): m/z=385.9, [M+H]⁺.

### Step 4. Preparation of N-(1-(4-methoxy-3-(1-methyl-1H-pyrazol-4-yl)naphthalen-1-yl)ethyl)-2-methylpropane-2-sulfinamide

To a solution of N-(1-(3-bromo-4-methoxynaphthalen-1-yl)ethyl)-2-methylpropane-2-sulfinamide (1800.0 mg, 4.683 mmol) in 1,4-dioxane (60.0 mL) under N₂ were added sat. aq. Na₂CO₃ (10.0 mL) and

Pd(dppf)Cl₂ (343 mg, 0.468 mmol), and 1-methyl-4-(4,4,5,5-tetramethyl-1,3,2-dioxaborolan-2-yl)-1H-pyrazole (1950 mg, 9.37 mmol) at 20° C. The reaction mixture was stirred at 100° C. under N₂ for 15 h, at which time LCMS showed a mass peak of the desired product. The reaction mixture was then filtered, and the filtrate was evaporated in vacuo. The residue was purified by silica gel chromatography using a Biotage (20 g silica gel column, PE:EA=1:0 to 0:1) to afford N-(1-(4-methoxy-3-(1-methyl-1H-pyrazol-4-yl)naphthalen-1-yl)ethyl)-2-methylpropane-2-sulfinamide (1432 mg, 79.3%) as a yellow oil. LCMS (ESI): m/z=386.2, [M+H]⁺.

### Step 5. Preparation of 1-(4-methoxy-3-(1-methyl-1H-pyrazol-4-yl)naphthalen-1-yl)ethan-1-amine

To a solution of N-(1-(4-methoxy-3-(1-methyl-1H-pyrazol-4-yl)naphthalen-1-yl)ethyl)-2-methylpropane-2-sulfinamide (1400 mg, 3.631 mmol) in MeOH (30 mL) was added HCl (3 mL, conc.) at 0° C. The reaction mixture was stirred at 20° C. for 15 h, at which time LCMS showed a mass peak of the desired product. The reaction mixture was then evaporated in vacuo then partitioned between ethyl acetate and sat. aq. Na₂CO₃ (100/50 mL). The organic layer was separated, and the aqueous layer was re-extracted with ethyl acetate (50 mL). The combined organic layer was washed with brine, dried with anhydrous Na₂SO₄, and evaporated in vacuo to afford 1-(4-methoxy-3-(1-methyl-1H-pyrazol-4-yl)naphthalen-1-yl)ethan-1-amine (985 mg, 96.4%) as a red oil. LCMS (ESI): m/z=265.1, [M+H−NH₃]⁺.

### Preparation P16: 5-(4-(tert-butoxycarbonyl)piperazin-1-yl)-2-methylbenzoic acid

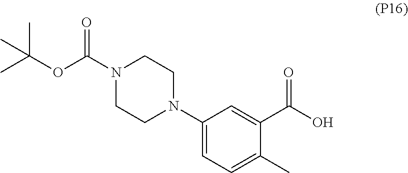 [SMILES: Cc1ccc(N2CCN(C(=O)OC(C)(C)C)CC2)cc1C(=O)O]

5-(4-(tert-butoxycarbonyl)piperazin-1-yl)-2-methylbenzoic acid was synthesized following a procedure described in intermediate 18 in patent number CN114957165A, “Antiviral compound and preparation method and application thereof”.

### Preparation P17: 2-(3-hydrazineyl-3-oxopropyl)-N-(1-(2-(1-methyl-1H-pyrazol-4-yl)quinolin-4-yl)cyclopropyl)benzamide

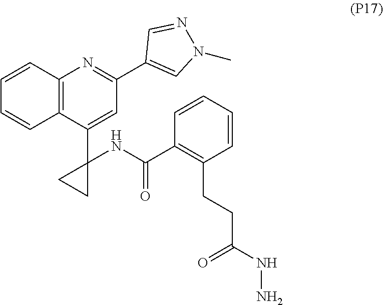 [SMILES: Cn1cc(-c2cc(C3(NC(=O)c4ccccc4CCC(=O)NN)CC3)c3ccccc3n2)cn1]

### Step 1. Preparation of methyl 3-(2-((1-(2-(1-methyl-1H-pyrazol-4-yl)quinolin-4-yl)cyclopropyl)carbamoyl)phenyl)propanoate

To a solution of 2-(3-methoxy-3-oxopropyl)benzoic acid (394 mg, 1.89 mmol) in DMF (15 mL) were added HATU (1080 mg, 2.84 mmol) and TEA (3 mL), followed by 1-(2-(1-methyl-1H-pyrazol-4-yl)quinolin-4-yl)cyclopropan-1-amine (500 mg, 1.89 mmol) at 20° C. The reaction mixture was stirred at 20° C. for 15 h, at which time LCMS showed a mass peak of the desired product. The reaction mixture was then partitioned between ethyl acetate and H₂O (100/50 mL). The organic layer was separated, and the aqueous layer was re-extracted with ethyl acetate (50 mL). The combined organic layer was washed with brine, dried with anhydrous Na₂SO₄, and evaporated in vacuo to afford methyl 3-(2-((1-(2-(1-methyl-1H-pyrazol-4-yl)quinolin-4-yl)cyclopropyl)carbamoyl)phenyl)propanoate (860 mg, 100%) as a yellow solid. LCMS (ESI): m/z=455.2, [M+H]⁺.

### Step 2. Preparation of 2-(3-hydrazineyl-3-oxopropyl)-N-(1-(2-(1-methyl-1H-pyrazol-4-yl)quinolin-4-yl)cyclopropyl)benzamide

To a solution of methyl 3-(2-((1-(2-(1-methyl-1H-pyrazol-4-yl)quinolin-4-yl)cyclopropyl)carbamoyl)phenyl)propanoate (860 mg, 1.89 mmol) in EtOH (20 mL) was added NH₂NH₂·H₂O (4060 mg, 81.10 mmol) at 20° C. The reaction mixture was stirred at 80° C. for 15 h, at which time LCMS showed a mass peak of the desired product. The reaction mixture was then partitioned between DCM and H₂O (50/50 mL). The organic layer was separated, and the aqueous layer was re-extracted with DCM (50 mL). The combined organic layer was washed with brine, dried with anhydrous Na₂SO₄, and evaporated in vacuo. The residue was then purified by silica gel chromatography using a Biotage (20 g silica gel column, PE:EA=1:0 to 0:1, EA:MeOH=1:0 to 0:1) to afford 2-(3-hydrazineyl-3-oxopropyl)-N-(1-(2-(1-methyl-1H-pyrazol-4-yl)quinolin-4-yl)cyclopropyl)benzamide (810 mg, 94.2% yield) as a yellow oil. LCMS (ESI): m/z=455.2, [M+H]⁺.

### Preparation P18: 2-methyl-4-(1-(thiazole-4-carboxamido)cyclopropyl)benzoic acid

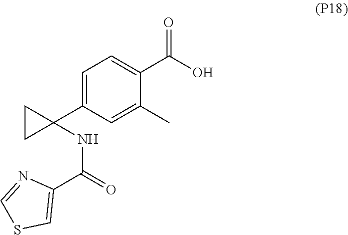 [SMILES: Cc1cc(C2(NC(=O)c3cscn3)CC2)ccc1C(=O)O]

### Step 1. Preparation of 1-(4-bromo-3-methylphenyl)cyclopropan-1-amine

To a stirred solution of 4-bromo-3-methylbenzonitrile (2000 mg, 10.20 mmol) and Ti(O-iPr)₄ (3190 mg, 11.2 mmol) in THF (50 mL) was added EtMgBr (7.48 mL, 22.44 mmol, 3 M) dropwise at −78° C. The reaction was warmed to 20° C., stirred for 1 h, treated with BF₃·Et₂O (2900 mg, 20.4 mmol), and kept at 20° C. for 0.5 h. After that, LCMS showed a mass peak of the desired product. The reaction was then quenched with 1N HCl (10 mL), stirred at 20° C. for 0.5 h, and partitioned between ethyl acetate and H₂O (100/50 mL). The organic layer was discarded, and the aqueous layer was basified with NH₃·H₂O to pH=9 at 0° C. and extracted with DCM (100 mL). The organic layer was washed with brine, dried with anhydrous Na₂SO₄, and evaporated in vacuo to afford 1-(4-bromo-3-methylphenyl)cyclopropan-1-amine (422 mg, 18.3%) as a yellow oil. LCMS (ESI): m/z=226.1, [M+H]⁺.

### Step 2. Preparation of N-(1-(4-bromo-3-methylphenyl)cyclopropyl)thiazole-4-carboxamide

To a solution of thiazole-4-carboxylic acid (228 mg, 1.77 mmol) in DMF (20 mL) at 20° C. were added HATU (1010 mg, 2.65 mmol) and TEA (3 mL), and then 1-(4-bromo-3-methylphenyl)cyclopropan-1-amine (400.0 mg, 1.77 mmol). The reaction mixture was stirred at 20° C. for 15 h, at which time LCMS showed a mass peak of the desired product. The reaction mixture was partitioned between DCM and H₂O (50/50 mL). The organic layer was separated and the aqueous layer was re-extracted with DCM (50 mL). The combined organic layer was washed with brine, dried with anhydrous Na₂SO₄, and evaporated in vacuo. The residue was then purified by silica gel chromatography using a Biotage (20 g silica gel column, PE:EA=1:0 to 0:1, EA:MeOH=1:0 to 0:1) to afford N-(1-(4-bromo-3-methylphenyl)cyclopropyl)thiazole-4-carboxamide (523 mg, 87.7%) as a yellow solid. LCMS (ESI): m/z=338.9, [M+H]⁺.

### Step 3. Preparation of ethyl 2-methyl-4-(1-(thiazole-4-carboxamido)cyclopropyl)benzoate

To a solution of N-(1-(4-bromo-3-methylphenyl)cyclopropyl)thiazole-4-carboxamide (300.0 mg, 0.890 mmol) in EtOH (20.0 mL) were added Mo(CO)₆ (235 mg, 0.890 mmol), Pd(OAc)₂ (20.0 mg, 0.0890 mmol), t-Bu₃P·BF₄ (25.8 mg, 0.0890 mmol), and DBU (203 mg, 1.33 mmol) at 20° C. The resulting suspension was degassed to remove oxygen by bubbling through nitrogen gas gently for 2 min. The mixture was then sealed in a microwave tube and irradiated in the microwave on a Biotage Smith Synthesizer at 120° C. for 1 h. LCMS showed a mass peak of the desired product. The reaction mixture was evaporated in vacuo and purified by silica gel chromatography via a Biotage (20 g silica gel column, PE:EA=1:0 to 0:1) to afford ethyl 2-methyl-4-(1-(thiazole-4-carboxamido)cyclopropyl)benzoate (185 mg, 62.9%) as a yellow solid. LCMS (ESI): m/z=331.1, [M+H]⁺.

### Step 4. Preparation of 2-methyl-4-(1-(thiazole-4-carboxamido)cyclopropyl)benzoic acid

To a solution of ethyl 2-methyl-4-(1-(thiazole-4-carboxamido)cyclopropyl)benzoate (150 mg, 0.454 mmol) in MeOH (10 mL) at 20° C. was added 10% aq. NaOH (3 mL). The reaction mixture was stirred at 60° C. for 15 h. LCMS showed a mass peak of the desired product. The reaction mixture was then acidified with 1 N HCl to pH=5 at 0° C. and partitioned between ethyl acetate and H₂O (50/50 mL). The organic layer was separated, and the aqueous layer was re-extracted with ethyl acetate (50 mL). The combined organic layer was washed with brine, dried with anhydrous Na₂SO₄, and evaporated in vacuo to afford 2-methyl-4-(1-(thiazole-4-carboxamido)cyclopropyl)benzoic acid (137 mg, 99.8%) as a white solid. LCMS (ESI): m/z=303.1, [M+H]⁺.

### Preparation P19: 4-(1-(1H-imidazole-2-carboxamido)cyclopropyl)-2-methylbenzoic acid

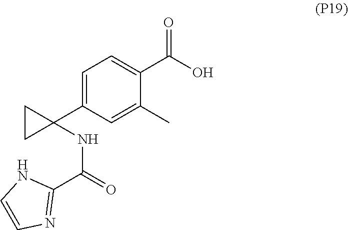 [SMILES: Cc1cc(C2(NC(=O)c3ncc[nH]3)CC2)ccc1C(=O)O]

Using a procedure analogous to the preparation of P18, Step 2-4 with 1-(4-bromo-3-methylphenyl)cyclopropan-1-amine (350.0 mg, 1.55 mmol) and 1H-imidazole-2-carboxylic acid (173 mg, 1.55 mmol) as the starting reactants affords 4-(1-(1H-imidazole-2-carboxamido)cyclopropyl)-2-methylbenzoic acid (51 mg final product, with 84.7%, 54.9%, and 47% yield in the three steps respectively). LCMS (ESI): m/z=286.1, [M+H]⁺.

### Preparation P20: 2-methyl-4-(2-(thiazole-4-carboxamido)propan-2-yl)benzoic acid

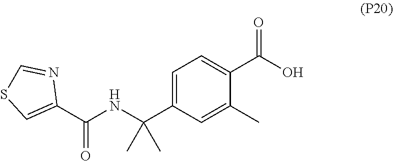 [SMILES: Cc1cc(C(C)(C)NC(=O)c2cscn2)ccc1C(=O)O]

### Step 1. Preparation of 2-(4-bromo-3-methylphenyl)propan-2-amine

To a stirred solution of 4-bromo-3-methylbenzonitrile (2000.0 mg, 10.20 mmol) in THF (40.0 mL) at 20° C. was added MeMgBr (10.0 mL, 3 M, 30 mmol) dropwise. The reaction mixture was stirred for 0.5 h. To the reaction mixture at 20° C. was then added Ti(O-iPr)₄ (2900 mg, 10.2 mmol), followed by stirring at 55° C. for 15 h. LCMS showed a mass peak of the desired product. The reaction was quenched with 1N HCl (10 mL), stirred at 20° C. for 0.5 h, then partitioned between ethyl acetate and H₂O (100/50 mL). The organic layer was discarded, and the aqueous layer was basified with NH₃·H₂O to pH=9 at 0° C. and extracted with DCM (100 mL). The organic layer was washed with brine, dried with anhydrous Na₂SO₄, and evaporated in vacuo to afford 2-(4-bromo-3-methylphenyl)propan-2-amine (525 mg, 22.6%) as a yellow oil. LCMS (ESI): m/z=213.1, [M+H−NH₃]⁺.

### Step 2-4. Preparation of 2-methyl-4-(2-(thiazole-4-carboxamido)propan-2-yl)benzoic acid

Using a procedure analogous to the preparation of P18, Step 2-4 with 2-(4-bromo-3-methylphenyl)propan-2-amine (200.0 mg, 0.877 mmol) and (113 mg, 0.877 mmol) as the reactants affords 2-methyl-4-(2-(thiazole-4-carboxamido)propan-2-yl)benzoic acid (137 mg final product, with 99.9%, 60.5%, 99.8% yield in the three steps respectively) as a white solid. LCMS (ESI): m/z=305.1, [M+H]⁺.

### Preparation P21: 4-(2-(1H-imidazole-2-carboxamido)propan-2-yl)-2-methylbenzoic acid

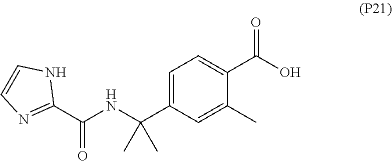 [SMILES: Cc1cc(C(C)(C)NC(=O)c2ncc[nH]2)ccc1C(=O)O]

Using a procedure analogous to the preparation of P18, Step 2-4 with 2-(4-bromo-3-methylphenyl)propan-2-amine (300.0 mg, 1.32 mmol) and 1H-imidazole-2-carboxylic acid (147 mg, 1.32 mmol) as the starting reactant affords 4-(2-(1H-imidazole-2-carboxamido)propan-2-yl)-2-methylbenzoic acid (70 mg final product, with 99.1%, 62.8%, and 51% yield in the three steps respectively) as a white solid. LCMS (ESI): m/z=288.1, [M+H]⁺.

### Preparation P22: (R)-3-methyl-4-((1-(2-(1-methyl-1H-pyrazol-4-yl)quinolin-4-yl)ethyl)carbamoyl)benzoic acid

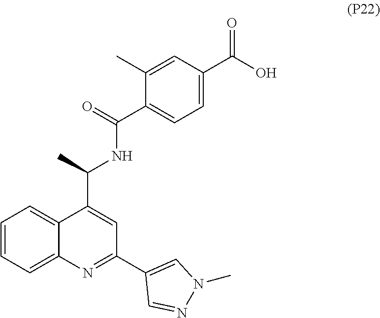 [SMILES: Cc1cc(C(=O)O)ccc1C(=O)N[C@H](C)c1cc(-c2cnn(C)c2)nc2ccccc12]

### Step 1. Preparation of methyl(R)-3-methyl-4-((1-(2-(1-methyl-1H-pyrazol-4-yl)quinolin-4-yl)ethyl)carbamoyl)benzoate

To a mixture of 4-(methoxycarbonyl)-2-methylbenzoic acid (385 mg, 1.98 mmol) in DMF (6.94 mL), DIEA (800 mg, 6 mmol), and HATU (754 mg, 1.98 mmol) was added (R)-1-(2-(1-methyl-1H-pyrazol-4-yl)quinolin-4-yl)ethan-1-amine (500.0 mg, 1.98 mmol), and the reaction mixture was stirred at 25° C. for 16 h. LCMS showed the desired product was observed as a major peak. The reaction mixture was then partitioned between ethyl acetate and H₂O (20/20 mL). The organic layer was separated, and the aqueous layer was re-extracted with ethyl acetate (10 mL). The combined organic layer was washed with brine, dried with anhydrous Na₂SO₄, and evaporated in vacuo. The residue was purified by silica gel chromatography via Biotage (12 g silica gel column, EA) to afford methyl(R)-3-methyl-4-((1-(2-(1-methyl-1H-pyrazol-4-yl)quinolin-4-yl)ethyl)carbamoyl)benzoate (638 mg, 75.1%) as a white solid. LCMS (ESI): m/z=429.2, [M+H]⁺.

### Step 2. Preparation of (R)-3-methyl-4-((1-(2-(1-methyl-1H-pyrazol-4-yl)quinolin-4-yl)ethyl)carbamoyl)benzoic acid

To a solution of methyl(R)-3-methyl-4-((1-(2-(1-methyl-1H-pyrazol-4-yl)quinolin-4-yl)ethyl) carbamoyl)benzoate (630 mg, 1.47 mmol) in THF (2.0 mL) at 25° C. were added LiOH·H₂O (308 mg, 7.35 mmol), MeOH (2.0 mL), and H₂O (2.0 mL). The reaction mixture was stirred at 25° C. for 12 h. LCMS showed that the desired product was observed as a major peak. The reaction was then acidified with 1N HCl to pH=6 at 0° C., evaporated in vacuo, and partitioned between DCM and H₂O (10/10 mL). The organic layer was separated, and the aqueous layer was re-extracted with DCM (5 mL×3). The DCM layers were dried over Na₂SO₄, filtered, and evaporated to give (R)-3-methyl-4-((1-(2-(1-methyl-1H-pyrazol-4-yl)quinolin-4-yl)ethyl)carbamoyl)benzoic acid (483.6 mg, 79.4%) as a white solid. LCMS (ESI): m/z=415.2, [M+H]⁺.

### Preparation P23: 4-(((tert-butoxycarbonyl)(thiazol-4-ylmethyl)amino)methyl)-2-methylbenzoic acid

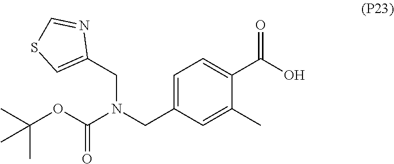 [SMILES: Cc1cc(CN(Cc2cscn2)C(=O)OC(C)(C)C)ccc1C(=O)O]

### Step 1. Preparation of methyl 2-methyl-4-(((thiazol-4-ylmethyl)amino)methyl)benzoate

To a solution of methyl 4-(aminomethyl)-2-methylbenzoate (1000.0 mg, 5.580 mmol) in MeOH (40 mL) were added thiazole-4-carbaldehyde (347 mg, 3.07 mmol), NaBH₃CN (701 mg, 11.2 mmol) and HOAc (1.5 mL). After 1 h of stirring at 20° C., LCMS showed a mass peak of the desired product. The reaction mixture was then partitioned between ethyl acetate and H₂O (50/50 mL). The organic layer was separated and the aqueous layer was re-extracted with ethyl acetate (50 mL). The combined organic layer was washed with brine, dried with anhydrous Na₂SO₄, and evaporated in vacuo to afford the methyl 2-methyl-4-(((thiazol-4-ylmethyl)amino)methyl)benzoate (1200 mg, 77.9%) as a yellow oil. LCMS (ESI): m/z=277.2, [M+H]⁺.

### Step 2. Preparation of methyl 4-(((tert-butoxycarbonyl)(thiazol-4-ylmethyl)amino)methyl)-2-methylbenzoate

To solution of methyl 2-methyl-4-(((thiazol-4-ylmethyl)amino)methyl)benzoate (1200.0 mg, 4.342 mmol) in DCM (20 mL) were added Boc₂O (2840 mg, 13.0 mmol) and TEA (2200 mg, 21.7 mmol). The reaction mixture was stirred at 20° C. for 15 h then partitioned between DCM and H₂O (50/50 mL). The organic layer was separated, and the aqueous layer was re-extracted with DCM (50 mL). The combined organic layer was washed with brine, dried over Na₂SO₄, and evaporated in vacuo. The residue was then purified by silica gel chromatography via Biotage (20 g silica gel column, PE:EA=1:0 to 0.7:0.3) to afford the product methyl 4-(((tert-butoxycarbonyl)(thiazol-4-ylmethyl)amino)methyl)-2-methylbenzoate (690 mg, 42.2%) as a yellow oil. LCMS (ESI): m/z=399.1, [M+Na]⁺.

### Step 3. Preparation of 4-(((tert-butoxycarbonyl)(thiazol-4-ylmethyl)amino)methyl)-2-methylbenzoic acid

To a solution of methyl 4-(((tert-butoxycarbonyl)(thiazol-4-ylmethyl)amino)methyl)-2-methylbenzoate (690.0 mg, 1.83 mmol) in MeOH (10.0 mL) at 20° C. was added aq. 10% NaOH solution (5.0 mL). The mixture was stirred at 70° C. for 16 h, at which time LCMS showed a mass peak of the desired product. The mixture was evaporated in vacuo, then pH was adjusted to about 6 with 1N HCl, and extracted with ethyl acetate (20 mL×3). The combined organic layer was washed with brine, dried over Na₂SO₄ and evaporated in vacuo to afford 4-(((tert-butoxycarbonyl)(thiazol-4-ylmethyl)amino)methyl)-2-methylbenzoic acid (660 mg, 99.4%) as a yellow solid. LCMS (ESI): m/z=385.1, [M+Na]⁺.

### Preparation P25: 5-(6-(tert-butoxycarbonyl)-1-oxo-2,6-diazaspiro[4.5]decan-2-yl)-2-methylbenzoic acid

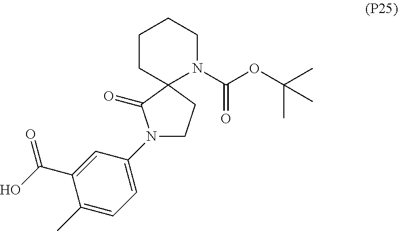 [SMILES: Cc1ccc(N2CCC3(CCCCN3C(=O)OC(C)(C)C)C2=O)cc1C(=O)O]

### Step 1. Preparation of tert-butyl 2-(3-(methoxycarbonyl)-4-methylphenyl)-1-oxo-2,6-diazaspiro[4.5]decane-6-carboxylate

To a solution of methyl 5-bromo-2-methylbenzoate (99.10 mg, 0.433 mmol) in DMF (3.00 mL) were added CuI (7.49 mg, 0.0393 mmol), tert-butyl 1-oxo-2,6-diazaspiro[4.5]decane-6-carboxylate (100.00 mg, 0.393 mmol), N,N-dimethylglycine (8.11 mg, 0.0786 mmol), and K₂CO₃ (109.00 mg, 0.786 mmol). The mixture was bubbled with N₂ three times then stirred at 120° C. for 15 h. After that, the reaction mixture was partitioned between ethyl acetate and H₂O (5/2 mL). The organic layer was separated, and the aqueous layer was re-extracted with ethyl acetate (5 mL). The combined organic layer was washed with brine (5 mL×2), dried with anhydrous Na₂SO₄, and evaporated in vacuo. The residue was purified by column chromatography eluting with PE/EA (0-30%/100%-70%) to afford tert-butyl 2-(3-(methoxycarbonyl)-4-methylphenyl)-1-oxo-2,6-diazaspiro[4.5]decane-6-carboxylate (70.00 mg, 44%) as a yellow oil. LCMS (ESI): m/z=303.4, [M+H-Boc]⁺.

### Step 2. Preparation of 5-(6-(tert-butoxycarbonyl)-1-oxo-2,6-diazaspiro[4.5]decan-2-yl)-2-methylbenzoic acid

To a solution of tert-butyl 2-(3-(methoxycarbonyl)-4-methylphenyl)-1-oxo-2,6-diazaspiro[4.5]decane-6-carboxylate (70.0 mg, 0.17 mmol) in CH₃OH (2.0 mL) and H₂O (0.5 mL) at 20° C. was added NaOH (27.8 mg, 0.696 mmol), and the reaction was stirred at 20° C. for 16 h. LCMS showed that the starting material was consumed and a peak with a desired mass was observed. The reaction mixture was evaporated in vacuo to give a crude, which was then dissolved in H₂O (5 mL), acidized with aq. HCl(1N) to pH=5-6, and extracted with DCM (5 mL×3). The organic layer was evaporated in vacuo give 5-(6-(tert-butoxycarbonyl)-1-oxo-2,6-diazaspiro[4.5]decan-2-yl)-2-methylbenzoic acid (34 mg, 50%) as a yellow solid. LCMS (ESI): m/z=289.3, [M+H−Boc]⁺.

### Preparation P26: 2-methyl-4-(2-((thiazol-4-ylmethyl)amino)propan-2-yl)benzoic acid

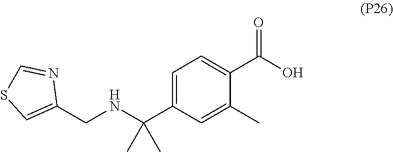 [SMILES: Cc1cc(C(C)(C)NCc2cscn2)ccc1C(=O)O]

### Step 1. Preparation of 2-(4-bromo-3-methylphenyl)-N-(thiazol-4-ylmethyl)propan-2-amine

To a solution of 2-(4-bromo-3-methylphenyl)propan-2-amine (690.0 mg, 3.02 mmol) and thiazole-4-carbaldehyde (274 mg, 2.42 mmol) in MeOH (15 mL) at 20° C. were added HOAc (0.5 mL) and NaBH₃CN (380 mg, 6.05 mmol). The reaction mixture was stirred at 20° C. for 15 h. LCMS showed a mass peak of the desired product. The reaction mixture was partitioned between DCM and sat. aq. Na₂CO₃ (50/50 mL). The organic layer was separated, and the aqueous layer was re-extracted with DCM (50 mL). The combined organic layer was washed with brine, dried with anhydrous Na₂SO₄, and evaporated in vacuo to afford a crude of 2-(4-bromo-3-methylphenyl)-N-(thiazol-4-ylmethyl)propan-2-amine (984 mg) as a yellow oil, which was used in the next step directly. LCMS (ESI): m/z=326.9, [M+H]⁺.

### Step 2-3. Preparation of 2-methyl-4-(2-((thiazol-4-ylmethyl)amino)propan-2-yl)benzoic acid

Using a procedure analogous to the preparation of P18, Step 3-4 with 2-(4-bromo-3-methylphenyl)-N-(thiazol-4-ylmethyl)propan-2-amine (984 mg, prepared above) as the reactant affords 2-methyl-4-(2-((thiazol-4-ylmethyl)amino)propan-2-yl)benzoic acid (274 mg) as a yellow solid. LCMS (ESI): m/z=291.1, [M+H]⁺.

### Preparation of P27: 5-(3-((tert-butoxycarbonyl)amino)-2-oxopyrrolidin-1-yl)-2-methylbenzoic acid

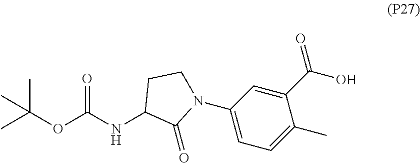 [SMILES: Cc1ccc(N2CCC(NC(=O)OC(C)(C)C)C2=O)cc1C(=O)O]

5-(3-((tert-butoxycarbonyl)amino)-2-oxopyrrolidin-1-yl)-2-methylbenzoic acid was prepared in a similar manner to 5-(6-(tert-butoxycarbonyl)-1-oxo-2,6-diazaspiro[4.5]decan-2-yl)-2-methylbenzoic acid (P25) using methyl 5-bromo-2-methylbenzoate and tert-butyl(2-oxopyrrolidin-3-yl)carbamate. LCMS (ESI): m/z=279.2, [M+H−tBu]⁺.

### Preparation of P28: 3-methyl-4-((1-(2-(1-methyl-1H-pyrazol-4-yl)quinolin-4-yl)cyclopropyl)carbamoyl)benzoic acid

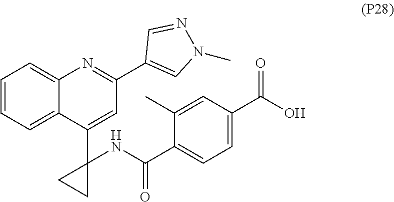 [SMILES: Cc1cc(C(=O)O)ccc1C(=O)NC1(c2cc(-c3cnn(C)c3)nc3ccccc23)CC1]

3-methyl-4-((1-(2-(1-methyl-1H-pyrazol-4-yl)quinolin-4-yl)cyclopropyl)carbamoyl)benzoic acid was prepared in a similar manner to the preparation of (R)-2-(3-methyl-4-((1-(2-(1-methyl-1H-pyrazol-4-yl)quinolin-4-yl)ethyl)carbamoyl)phenyl)acetic acid (Example 200) in step 5-6 from 1-(2-(1-methyl-1H-pyrazol-4-yl)quinolin-4-yl)cyclopropan-1-amine and 4-(methoxycarbonyl)-2-methylbenzoic acid. LCMS (ESI): m/z=427.1, [M+H]⁺.

### Preparation of P29: 2-methyl-4-(1-(thiazole-4-carboxamido)ethyl)benzoic acid

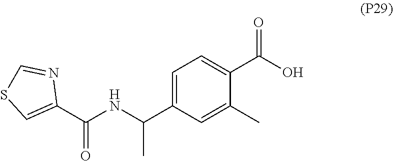 [SMILES: Cc1cc(C(C)NC(=O)c2cscn2)ccc1C(=O)O]

### Step 1. Preparation of methyl 4-formyl-2-methylbenzoate

To a solution of 4-bromo-3-methylbenzaldehyde (1.0 g, 5 mmol) and TEA (1530 mg, 15.1 mmol) in MeOH (20.0 mL) at 20° C. was added.

Pd(dppf)Cl₂ (368 mg, 0.502 mmol). The mixture was purged with Argon three times then CO gas three times, and then kept at 65° C. for 15 h at a pressure of 50 psi (3.44738 bar). The reaction mixture was filtered and evaporated in vauo to give methyl 4-formyl-2-methylbenzoate (450 mg, 50%) as a brown oil. ¹H NMR (400 MHz, CDCl₃) δ=10.08-10.02 (m, 1H), 8.03 (d, J=8.4 Hz, 1H), 7.81-7.70 (m, 2H), 3.96-3.91 (m, 3H), 2.67 (s, 3H). LCMS (ESI): m/z=179.1, [M+H]⁺.

### Step 2. Preparation of methyl (E)-4-(((tert-butylsulfinyl)imino)methyl)-2-methylbenzoate

To a solution of methyl 4-formyl-2-methylbenzoate (450.0 mg, 2.53 mmol) and 2-methylpropane-2-sulfinamide (918 mg, 7.58 mmol) in DCM (10.0 mL) was added Cs₂CO₃ (1230 mg, 3.79 mmol) at 20° C. The mixture was stirred at 20° C. for 12 h then partitioned between DCM and H₂O (50/50 mL). The organic layer was separated, and the aqueous layer was re-extracted with DCM (30 mL). The combined organic layer was wash with brine (50 mL), dried over Na₂SO₄, filtered, and concentrated to give methyl (E)-4-(((tert-butylsulfinyl)imino)methyl)-2-methylbenzoate (650 mg, 91.5%) as a yellow oil. ¹H NMR (400 MHz, CDCl₃) δ=8.57 (s, 1H), 7.97 (d, J=8.6 Hz, 1H), 7.73-7.66 (m, 2H), 3.91 (s, 3H), 2.64 (s, 3H), 1.26 (s, 9H). LCMS (ESI): m/z=282.1, [M+H]⁺.

### Step 3. Preparation of methyl 4-(1-((tert-butylsulfinyl)amino)ethyl)-2-methylbenzoate

To a solution of methyl (E)-4-(((tert-butylsulfinyl)imino)methyl)-2-methylbenzoate (650.0 mg, 2.31 mmol) in THF (10.0 mL) at 0° C. under N₂, was added slowly 3 M MeMgBr (2.3 mL, 6.93 mmol). The reaction mixture was stirred at 20° C. under N₂ for 15 h. The reaction was then quenched by water (10 mL) at 0° C. and extracted with ethyl acetate (20 mL×3). The combined organic layer was washed with brine (10 mL), dried over Na₂SO₄, evaporated in vacuo to give a crude, which was purified by silica gel chromatography using a Biotage (12 g silica gel column, EA:PE=1:100 to 3:2) to afford methyl 4-(1-((tert-butylsulfinyl)amino)ethyl)-2-methylbenzoate (100 mg, 12.6%) as a yellow solid. ¹H NMR (400 MHz, CDCl₃) δ=7.93-7.87 (m, 1H), 7.25-7.18 (m, 2H), 4.65-4.48 (m, 1H), 3.91-3.85 (m, 3H), 2.63-2.56 (m, 3H), 1.85-1.73 (m, 1H), 1.56-1.49 (m, 3H), 1.21 (s, 7H). LCMS (ESI): m/z=298.1, [M+H]⁺.

### Step 4. Preparation of methyl 4-(1-aminoethyl)-2-methylbenzoate

To a solution of methyl 4-(1-((tert-butylsulfinyl)amino)ethyl)-2-methylbenzoate (100.0 mg, 0.336 mmol) in DCM (4.0 mL) was added HCl in MeOH (2 mL) at 20° C. The reaction mixture was stirred at 20° C. for 0.5 h then evaporated in vacuo to give methyl 4-(1-aminoethyl)-2-methylbenzoate (100 mg, 98.2%) as a colorless oil. ¹H NMR (400 MHz, CDCl₃-d4) δ=7.89 (d, J=7.9 Hz, 1H), 7.37-7.21 (m, 2H), 4.07 (q, J=6.7 Hz, 1H), 3.93-3.85 (m, 3H), 2.61 (s, 3H), 1.42 (d, J=6.6 Hz, 3H). LCMS (ESI): m/z=194.2, [M+H]⁺.

### Step 5. Preparation of methyl 2-methyl-4-(1-(thiazole-4-carboxamido)ethyl)benzoate

Using a similar procedure to Step 2 of Preparation of P18, from thiazole-4-carboxylic acid and methyl 4-(1-aminoethyl)-2-methylbenzoate, affords methyl 2-methyl-4-(1-(thiazole-4-carboxamido)ethyl)benzoate as a yellow solid. ¹H NMR (400 MHz, MeOD-d₄-d4) δ=9.07-9.01 (m, 1H), 8.25 (d, J=2.0 Hz, 1H), 7.95-7.82 (m, 1H), 7.37-7.25 (m, 2H), 5.24 (q, J=6.9 Hz, 1H), 3.87 (s, 3H), 2.58 (s, 3H), 1.59 (d, J=7.1 Hz, 3H). LCMS (ESI): m/z=305.2 [M+H]⁺.

### Step 6. Preparation of 2-methyl-4-(1-(thiazole-4-carboxamido)ethyl)benzoic acid

Using a similar procedure to Step 4 of Preparation of P18, from methyl 2-methyl-4-(1-(thiazole-4-carboxamido)ethyl)benzoate, affords 2-methyl-4-(1-(thiazole-4-carboxamido)ethyl)benzoic acid. LCMS (ESI): m/z=291.1, [M+H]⁺.

### Preparation of P30: 4-(1-(1H-imidazole-2-carboxamido)ethyl)-2-methylbenzoic acid

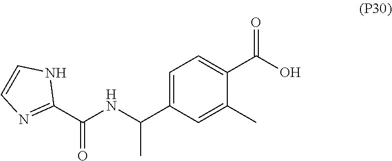 [SMILES: Cc1cc(C(C)NC(=O)c2ncc[nH]2)ccc1C(=O)O]

Using a similar procedure to Step 5-6 of Preparation of P29, from methyl 4-(1-aminoethyl)-2-methylbenzoate and 1H-imidazole-2-carboxylic acid, affords 4-(1-(1H-imidazole-2-carboxamido)ethyl)-2-methylbenzoic acid. LCMS (ESI): m/z=274.3, [M+H]⁺.

### Preparation of P31: (R)-2-methyl-N-(1-(2-(1-methyl-1H-pyrazol-4-yl)quinolin-4-yl)ethyl)-4-((methylamino)methyl)benzamide

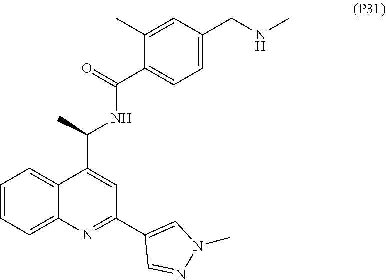 [SMILES: CNCc1ccc(C(=O)N[C@H](C)c2cc(-c3cnn(C)c3)nc3ccccc23)c(C)c1]

### Step 1. Preparation of methyl 4-(aminomethyl)-2-methylbenzoate

To a solution of methyl 4-cyano-2-methylbenzoate (4000 mg, 22.83 mmol) in MeOH (50 mL) and THF (50.0 mL) were added ammonium hydroxide (10 mL) and Raney Ni (1960 mg, 22.8 mmol). The mixture was purged and refilled with H₂ 3 times then stirred under H₂ (15 psi, i.e. 1.03421 bar) at 20° C. for 16 h. LCMS showed a mass peak of the desired product. The reaction was evaporated in vacuo then partitioned between ethyl acetate and H₂O (100/50 mL). The organic layer was separated, and the aqueous layer was re-extracted with ethyl acetate (50 mL). The combined organic layer was washed with brine, dried with anhydrydrous Na₂SO₄, and evaporated in vacuo to afford methyl 4-(aminomethyl)-2-methylbenzoate (3000 mg, 73.3%) as a yellow solid. LCMS (ESI): m/z=180.3, [M+H]⁺.

### Step 2. Preparation of methyl 4-(((tert-butoxycarbonyl)amino)methyl)-2-methylbenzoate

To a solution of methyl 4-(aminomethyl)-2-methylbenzoate (1000.0 mg, 5.580 mmol) in DCM (40.0 mL) were added Boc₂O (1830 mg, 8.37 mmol) and TEA (1690 mg, 16.7 mmol). The reaction was stirred at 20° C. for 15 h. LCMS showed a mass peak of the desired product, then reaction mixture was partitioned between DCM and H₂O (20/50 mL). The organic layer was separated, and the aqueous layer was re-extracted with DCM (50 mL). The combined organic layer was washed with brine, dried with anhydrous Na₂SO₄, and evaporated in vacuo to give a crude, which was purified by silica gel chromatography using a Biotage (20 g silica gel column, PE:EA=1:0 to 0.7:0.3) to afford methyl 4-(((tert-butoxycarbonyl)amino)methyl)-2-methylbenzoate (1400 mg, 89.8%) as a yellow oil. LCMS (ESI): m/z=224.4, [M+H−tBu]⁺.

### Step 3. Preparation of methyl 4-(((tert-butoxycarbonyl)(methyl)amino)methyl)-2-methylbenzoate

To a solution of methyl 4-(((tert-butoxycarbonyl)amino)methyl)-2-methylbenzoate (300.0 mg, 1.07 mmol) in THF (20 mL) at 0° C. was added t-BuOK (181 mg, 1.61 mmol). The mixture was stirred at 25° C. for 0.5 h, then MeI (229 mg, 1.61 mmol) was added dropwise to the reaction at 0° C. After that, the reaction mixture was stirred at 25° C. for 15 h, quenched with H₂O (30 mL), and extracted with ethyl acetate (30 mL×3). The combined organic layer was washed with brine, dried with anhydrous Na₂SO₄, and evaporated in vacuo to afford methyl 4-(((tert-butoxycarbonyl)(methyl)amino)methyl)-2-methylbenzoate (300 mg, 95.2%) as a brown oil. LCMS (ESI): m/z=238.3, [M+H−tBu]⁺.

### Step 4. Preparation of 4-(((tert-butoxycarbonyl)(methyl)amino)methyl)-2-methylbenzoic acid

To a solution of methyl 4-(((tert-butoxycarbonyl)(methyl)amino)methyl)-2-methylbenzoate (300.0 mg, 1.02 mmol) in MeOH (20.0 mL) was added aq. 10% NaOH solution (10.0 mL) at 20° C. The mixture was stirred at 70° C. for 16 h before being evaporated in vacuo to remove the MeOH, and adjusted to pH=−6 with 1N HCl, and extracted with ethyl acetate (20 mL×3). The combined organic layer was washed with brine, dried over Na₂SO₄, and evaporated in vacuo to afford 4-(((tert-butoxycarbonyl)(methyl)amino)methyl)-2-methylbenzoic acid (300 mg, 105%) as a yellow solid. LCMS (ESI): m/z=224.2, [M+H−tBu]⁺.

### Step 5. Preparation of tert-butyl(R)-methyl(3-methyl-4-((1-(2-(1-methyl-1H-pyrazol-4-yl)quinolin-4-yl)ethyl)carbamoyl)benzyl)carbamate

Using a similar procedure to Step 1 of Example 4, reaction of 4-(((tert-butoxycarbonyl)(methyl)amino)methyl)-2-methylbenzoic acid and (R)-1-(2-(1-methyl-1H-pyrazol-4-yl)quinolin-4-yl)ethan-1-amine affords tert-butyl(R)-methyl(3-methyl-4-((1-(2-(1-methyl-1H-pyrazol-4-yl)quinolin-4-yl)ethyl)carbamoyl)benzyl)carbamate as a yellow oil. LCMS (ESI): m/z=514.3, [M+H]⁺.

### Step 6. Preparation of (R)-2-methyl-N-(1-(2-(1-methyl-1H-pyrazol-4-yl)quinolin-4-yl)ethyl)-4-((methylamino)methyl)benzamide

Using a similar procedure to Step 2 of Example 4, from tert-butyl(R)-methyl(3-methyl-4-((1-(2-(1-methyl-1H-pyrazol-4-yl)quinolin-4-yl)ethyl)carbamoyl)benzyl)carbamate affords (R)-2-methyl-N-(1-(2-(1-methyl-1H-pyrazol-4-yl)quinolin-4-yl)ethyl)-4-((methylamino)methyl)benzamide. LCMS (ESI): m/z=414.2, [M+H]⁺.

### Preparation of P32: methyl(R)-4-(4-(1-(5-(((tert-butoxycarbonyl)amino)methyl)-2-methylbenzamido)ethyl)quinolin-2-yl)-1-methyl-1H-pyrrole-2-carboxylate

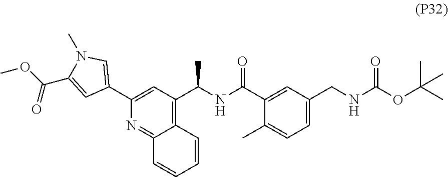 [SMILES: COC(=O)c1cc(-c2cc([C@@H](C)NC(=O)c3cc(CNC(=O)OC(C)(C)C)ccc3C)c3ccccc3n2)cn1C]

### Step 1. Preparation of methyl(R)-4-(4-(1-(((benzyloxy)carbonyl)amino)ethyl)quinolin-2-yl)-1-methyl-1H-pyrrole-2-carboxylate

To a solution of benzyl(R)-(1-(2-chloroquinolin-4-yl)ethyl)carbamate (1.0 g, 2.9 mmol) and methyl 1-methyl-4-(4,4,5,5-tetramethyl-1,3,2-dioxaborolan-2-yl)-1H-pyrrole-2-carboxylate (778 mg, 2.93 mmol) in 1,4-dioxane (5.0 mL) and sat. aq. Na₂CO₃ (1.0 mL) was added Pd(dppf)Cl₂ (215 mg, 0.293 mmol) at 20° C. The mixture was degassed and purged with N₂ 3 times, then stirred under N₂ balloon at 100° C. for 16 h. The reaction mixture was partitioned between ethyl acetate and H₂O (50/50 mL). The organic layer was separated, and the aqueous layer was re-extracted with ethyl acetate (50 mL). The combined organic layer was washed with brine (50 mL), dried over Na₂SO₄, filtered, and concentrated to give a residue, which was purified by silica gel chromatography via Biotage (20 g silica gel column, PE:EA=100:1 to 1:1) to afford methyl(R)-4-(4-(1-((tert-butoxycarbonyl)amino)ethyl)quinolin-2-yl)-1-methyl-1H-pyrrole-2-carboxylate (660 mg, 51.0%) as a white gum. ¹H NMR (400 MHz, CDCl₃) δ=8.13-7.95 (m, 2H), 7.72-7.62 (m, 2H), 7.57 (s, 1H), 7.54-7.44 (m, 2H), 7.36 (br s, 5H), 5.64 (br s, 1H), 5.33 (br d, J=6.6 Hz, 1H), 5.14 (br s, 2H), 4.01 (s, 3H), 3.88 (s, 3H), 1.64 (br d, J=6.7 Hz, 3H). LCMS (ESI): m/z=444.3, [M+H]⁺.

### Step 2. Preparation of methyl(R)-4-(4-(1-((tert-butoxycarbonyl)amino)ethyl)quinolin-2-yl)-1-methyl-1H-pyrrole-2-carboxylate

To a H₂ flushed solution of methyl(R)-4-(4-(1-(((benzyloxy)carbonyl)amino)ethyl)quinolin-2-yl)-1-methyl-1H-pyrrole-2-carboxylate (500 mg, 1.13 mmol) in MeOH (10.0 mL) were added (Boc)₂O (1230 mg, 5.64 mmol) and wet Pd/C (181 mg, 1.70 mmol), and the reaction was stirred at 25° C. at 15 psi (1.03421 bar) H₂ for 16 h. After that, the reaction mixture was filtered and concentrated under reduced pressure to give methyl(R)-4-(4-(1-((tert-butoxycarbonyl)amino)ethyl)quinolin-2-yl)-1-methyl-1H-pyrrole-2-carboxylate (480 mg, 104%) as a yellow solid. LCMS (ESI): m/z=410.2, [M+H]⁺.

### Step 3. Preparation of methyl(R)-4-(4-(1-aminoethyl)quinolin-2-yl)-1-methyl-1H-pyrrole-2-carboxylate

To a solution of methyl(R)-4-(4-(1-((tert-butoxycarbonyl)amino)ethyl)quinolin-2-yl)-1-methyl-1H-pyrrole-2-carboxylate (480.0 mg, 1.17 mmol) in DCM (6.0 mL) was added TFA (668 mg, 5.86 mmol), and the mixture was stirred at 20° C. for 12 h. LCMS showed that the starting material remained (64%), and the desired product was observed (27%); therefore, more TFA (668 mg, 5.86 mmol) was added to the reaction solution, and the reaction was stirred at 20° C. for an additional 4 h, at which time, LCMS showed no starting material remained and a major peak with a desired mass was observed. The reaction mixture was then concentrated under reduced pressure to give methyl(R)-4-(4-(1-aminoethyl)quinolin-2-yl)-1-methyl-1H-pyrrole-2-carboxylate (480 mg, 96.7%) as a brown oil. ¹H NMR (500 MHz, CDCl₃) δ=9.43-8.45 (m, 2H), 8.37 (br s, 1H), 8.12-8.00 (m, 2H), 7.86-7.80 (m, 1H), 7.79-7.73 (m, 2H), 7.53-7.46 (m, 1H), 5.31 (s, 1H), 3.87 (s, 3H), 3.72 (s, 2H), 1.34 (s, 3H). LCMS (ESI): m/z=310.2, [M+H]⁺.

### Step 4. Preparation of methyl(R)-4-(4-(1-(5-(((tert-butoxycarbonyl)amino)methyl)-2-methylbenzamido)ethyl)quinolin-2-yl)-1-methyl-1H-pyrrole-2-carboxylate

To a solution of methyl(R)-4-(4-(1-aminoethyl)quinolin-2-yl)-1-methyl-1H-pyrrole-2-carboxylate (250 mg, 0.808 mmol) and 5-(((tert-butoxycarbonyl)amino)methyl)-2-methylbenzoic acid (214 mg, 0.808 mmol) in DCM (5.0 mL) at 20° C. were added DIEA (313 mg, 2.42 mmol) and HATU (461 mg, 1.21 mmol), and the mixture was stirred at 20° C. for 2 h. LCMS showed no starting material remained and a major peak with desired mass was observed. The reaction mixture was partitioned between ethyl acetate and H₂O (20/20 mL), the organic layer was separated, and the aqueous layer was re-extracted with ethyl acetate (20 mL). The combined organic layer was washed with brine (20 mL), dried over Na₂SO₄, filtered and evaporated to give a residue, which was purified via prep-TLC (PE/EtOAc=1:1) to give methyl(R)-4-(4-(1-(5-(((tert-butoxycarbonyl)amino)methyl)-2-methylbenzamido)ethyl)quinolin-2-yl)-1-methyl-1H-pyrrole-2-carboxylate (350 mg, 98.3%) as a yellow oil. ¹H NMR (400 MHz, CDCl₃) δ=8.25 (br d, J=8.2 Hz, 1H), 8.17 (br d, J=7.9 Hz, 2H), 7.79-7.62 (m, 3H), 7.59-7.50 (m, 2H), 7.32 (br s, 1H), 7.24-7.21 (m, 1H), 7.21-7.13 (m, 2H), 5.04-4.80 (m, 1H), 4.27 (br s, 2H), 4.08-3.96 (m, 3H), 3.93-3.84 (m, 3H), 2.81 (s, 3H), 2.42-2.40 (m, 3H), 1.42 (s, 9H). LCMS (ESI): m/z=557.3, [M+H]⁺.

### Preparation P33: 3-(3-(1-methyl-1H-pyrazol-4-yl)naphthalen-1-yl)oxetan-3-amine

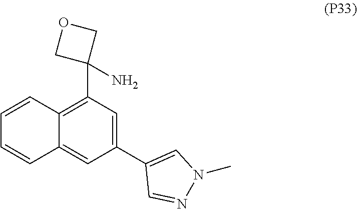 [SMILES: Cn1cc(-c2cc(C3(N)COC3)c3ccccc3c2)cn1]

### Step 1: Preparation of 2-methyl-N-(oxetan-3-ylidene)propane-2-sulfinamide

To a solution of oxetan-3-one (1000 mg, 13.88 mmol) and 2-methylpropane-2-sulfinamide (5050 mg, 41.6 mmol) in DCM (150 mL) was added Cs₂CO₃ (6780 mg, 20.8 mmol) at 20° C. The mixture was then stirred at 20° C. for 12 h before being diluted by water (50 mL) and extracted with ethyl acetate (100 mL×3). The combined organic layer was washed with brine (30 mL), dried over Na₂SO₄, filtered and evaporated in vacuo to give a residue, which was purified using a Biotage (12 g silica gel column, EA/PE from 0 to 30%) to give 2-methyl-N-(oxetan-3-ylidene)propane-2-sulfinamide (760 mg, 31.3%) as a yellow oil. ¹H NMR (400 MHz, DMSO-d6) δ=5.67-5.37 (m, 4H), 1.18 (s, 9H). LCMS (ESI): m/z=176.1, [M+H]+.

### Step 2: Preparation of N-(3-(3-bromonaphthalen-1-yl)oxetan-3-yl)-2-methylpropane-2-sulfinamide

To a solution of 1,3-dibromonaphthalene (490 mg, 1.71 mmol) in 2-methyltetrahydrofuran (9 mL) at −65° C. was added n-BuLi (1.71 mL, 1.71 mmol) at −65° C. under N₂ atmosphere. After stirring at −65° C. for 30 min, a solution of 2-methyl-N-(oxetan-3-ylidene)propane-2-sulfinamide (300 mg, 1.71 mmol) in 2-methyltetrahydrofuran (9 mL) was added dropwise to the reaction at −65° C. Then the reaction mixture was stirred at −65° C. for 15 min before being warmed up to 20° C. gradually and kept at 20° C. for 16 h. After that, the reaction was quenched by addition of sat. aq. NH₄Cl (30 mL) at 0° C., and the mixture was extracted with ethyl acetate (50 mL×3). The combined organic layer was washed with brine (20 mL), dried over Na₂SO₄, filtered, and evaporated in vacuo to give a residue, which was purified using a Biotage (40 g silica gel column, EA/PE from 0 to 100%) to give N-(3-(3-bromonaphthalen-1-yl)oxetan-3-yl)-2-methylpropane-2-sulfinamide (140 mg, 21.4%) as a white solid. ¹H NMR (400 MHz, DMSO-d6) δ=8.24 (d, J=1.5 Hz, 1H), 7.96 (br d, J=8.6 Hz, 1H), 7.68 (d, J=1.8 Hz, 1H), 7.60-7.51 (m, 3H), 6.63 (s, 1H), 5.42 (br d, J=6.8 Hz, 1H), 5.30 (d, J=6.4 Hz, 1H), 5.08 (d, J=7.0 Hz, 1H), 4.97-4.88 (m, 1H), 0.95 (s, 9H). LCMS (ESI): m/z=384.0, [M+H]+.

### Step 3: Preparation of 2-methyl-N-(3-(3-(1-methyl-1H-pyrazol-4-yl)naphthalen-1-yl)oxetan-3-yl)propane-2-sulfinamide

To a solution of N-(3-(3-bromonaphthalen-1-yl)oxetan-3-yl)-2-methylpropane-2-sulfinamide (140 mg, 0.350 mmol) and 1-methyl-4-(4,4,5,5-tetramethyl-1,3,2-dioxaborolan-2-yl)-1H-pyrazole (109 mg, 0.525 mmol) in 1,4-dioxane (4 mL) and H2O (1 mL) was added Na₂CO₃ (74.3 mg, 0.701 mmol). Then the mixture was bubbled with N₂ for 3 min before Pd(dppf)Cl₂ (25.6 mg, 0.0350 mmol) was added to it. The mixture was bubbled with N₂ for 3 min again then stirred at 100° C. for 15 h. After that, the reaction mixture was filtered. The filtrate was evaporated in vacuo, and the residue was purified by Biotage (4 g silica gel column, MeOH/EtOAc from 0 to 20%) to afford 2-methyl-N-(3-(3-(1-methyl-1H-pyrazol-4-yl)naphthalen-1-yl)oxetan-3-yl)propane-2-sulfinamide (230 mg, 171%) as a brown solid. ¹H NMR (400 MHz, DMSO-d6) δ=8.40 (s, 1H), 8.09 (s, 2H), 7.90 (d, J=8.0 Hz, 1H), 7.77 (s, 1H), 7.52-7.37 (m, 3H), 6.56 (s, 1H), 5.57 (br d, J=5.3 Hz, 1H), 5.34 (br d, J=6.4 Hz, 1H), 5.13 (d, J=6.9 Hz, 1H), 4.95 (br d, J=5.1 Hz, 1H), 3.91 (s, 3H), 0.94 (s, 9H). LCMS (ESI): m/z=384.1, [M+H]⁺.

### Step 4: Preparation of 3-(3-(1-methyl-1H-pyrazol-4-yl)naphthalen-1-yl)oxetan-3-amine

To a solution of 2-methyl-N-(3-(3-(1-methyl-1H-pyrazol-4-yl)naphthalen-1-yl)oxetan-3-yl)propane-2-sulfinamide (180.0 mg, 0.469 mmol) in DCM (5.0 mL) was added TFA (3800 mg, 34 mmol) at 20° C. The mixture was stirred at 20° C. for 0.5 h. Additional TFA (1540 mg, 13.5 mmol) was added to the reaction at 20° C. Then the mixture was stirred at 20° C. for 0.5 h. Then additional TFA (1540 mg, 13.5 mmol) was added to the reaction at 20° C. The mixture was stirred at 20° C. for 0.5 h. After that, the reaction mixture was diluted with water (10 mL) and extracted by DCM (20 mL×3). The aqueous layer was adjusted to pH=7-8 using NaHCO₃ then extracted by ethyl acetate (20 mL×3). The combined organic layer was washed with brine (10 mL), dried over Na₂SO₄, filtered and evaporated in vacuo to give 3-(3-(1-methyl-1H-pyrazol-4-yl)naphthalen-1-yl)oxetan-3-amine (120 mg, 91.5%) as a white solid. ¹H NMR (400 MHz, DMSO-d6) δ=8.33 (s, 1H), 8.07-7.97 (m, 2H), 7.89 (d, J=8.1 Hz, 1H), 7.69-7.57 (m, 2H), 7.51-7.37 (m, 2H), 5.19 (d, J=6.2 Hz, 2H), 4.86 (d, J=6.2 Hz, 2H), 3.90 (s, 3H). LCMS (ESI): m/z=280.1, [M+H]+.

### Preparation P34: 2-((3aR,6aS)-5-(tert-butoxycarbonyl)hexahydropyrrolo[3,4-c]pyrrol-2(1H)-yl)-5-methylisonicotinic acid

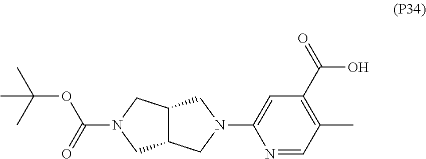 [SMILES: Cc1cnc(N2C[C@H]3CN(C(=O)OC(C)(C)C)C[C@H]3C2)cc1C(=O)O]

### Step 1: Preparation of tert-butyl(3aR,6aS)-5-(4-(ethoxycarbonyl)-5-methylpyridin-2-yl)hexahydropyrrolo[3,4-c]pyrrole-2(1H)-carboxylate

To a solution of ethyl 2-chloro-5-methylisonicotinate (200.0 mg, 1.00 mmol) in dioxane (20.0 mL) were added tert-butyl(3aR,6aS)-hexahydropyrrolo[3,4-c]pyrrole-2(1H)-carboxylate (425 mg, 2.00 mmol), Cs₂CO₃ (653 mg, 2.00 mmol), Ru-Phos (93.5 mg, 0.200 mmol), and Pd(OAc)₂ (22.5 mg, 0.100 mmol) at 25° C. The mixture was bubbled with N₂ for 1 min before being stirred at 100° C. for 15 h. The reaction mixture was then partitioned between ethyl acetate and H₂O (100/50 mL). The organic layer was separated and the aqueous layer was re-extracted with ethyl acetate (50 mL). The combined organic layer was washed with brine, dried with Na₂SO₄, evaporated in vacuo, and purified by silica gel chromatography using a Biotage (20 g silica column, PE:EtOAc=1:0 to 0:1) to afford tert-butyl(3aR,6aS)-5-(4-(ethoxycarbonyl)-5-methylpyridin-2-yl)hexahydropyrrolo[3,4-c]pyrrole-2(1H)-carboxylate (322 mg, 85.6%) as a yellow oil. LCMS (ESI): m/z=376.4, [M+H]⁺.

### Step 2: Preparation of 2-((3aR,6aS)-5-(tert-butoxycarbonyl)hexahydropyrrolo[3,4-c]pyrrol-2(1H)-yl)-5-methylisonicotinic acid

To a solution of tert-butyl(3aR,6aS)-5-(4-(ethoxycarbonyl)-5-methylpyridin-2-yl)hexahydropyrrolo[3,4-c]pyrrole-2(1H)-carboxylate (300 mg, 0.799 mmol) in MeOH (15 mL) was added NaOH (2 mL, 10% in aq.) at 20° C. The reaction mixture was stirred at 60° C. for 15 h. The reaction mixture was then acidified with 1N HCl to pH=7 at 0° C. The mixture was evaporated to remove the MeOH, and the aqueous layer was purified by prep-HPLC to afford 2-((3aR,6aS)-5-(tert-butoxycarbonyl)hexahydropyrrolo[3,4-c]pyrrol-2(1H)-yl)-5-methylisonicotinic acid (101 mg, 36.4%) as a white solid after lyophilization. LCMS (ESI): m/z=348.1, [M+H]⁺.

### Prep-HPLC Condition:

Column: Boston Prime C18 150*30 mm*5 μm;
Mobile phase: water (FA)-MeCN; B %: 0%-36%,
Flow rate (mL/min): 30 mL/min.

### Preparation P35: 5-(5-(tert-butoxycarbonyl)-2,5-diazabicyclo[2.2.1]heptan-2-yl)-2-methylbenzoic acid

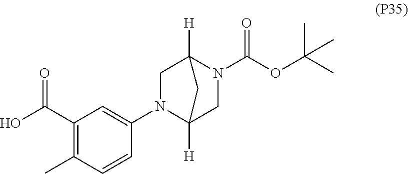 [SMILES: Cc1ccc(N2CC3CC2CN3C(=O)OC(C)(C)C)cc1C(=O)O]

### Step 1: Preparation of tert-butyl 5-(3-(methoxycarbonyl)-4-methylphenyl)-2,5-diazabicyclo[2.2.1]heptane-2-carboxylate

To a solution of methyl 5-bromo-2-methylbenzoate (800 mg, 3.49 mmol) and tert-butyl 2,5-diazabicyclo[2.2.1]heptane-2-carboxylate (692 mg, 3.49 mmol) in dioxane (34.9 mL) were added Ru-phos (326 mg, 0.698 mmol), Cs₂CO₃ (2280 mg, 6.98 mmol), and Pd(OAc)₂ (78.4 mg, 0.349 mmol) at 25° C. The mixture was bubbled with N₂ for 1 min before being stirred at 100° C. for 20 h. The reaction mixture was then diluted with H₂O (30 mL) and extracted with ethyl acetate (15 mL×3). The combined organic layer was dried over Na₂SO₄ and evaporated in vacuo to give a residue, which was purified by chromatography on silica gel (20 g, EA/PE=0-100%) to give tert-butyl 5-(3-(methoxycarbonyl)-4-methylphenyl)-2,5-diazabicyclo[2.2.1]heptane-2-carboxylate (1056 mg, 87.3%) as a yellow gum. LCMS (ESI): m/z=347.1, [M+H]+.

### Step 2: Preparation of 5-(5-(tert-butoxycarbonyl)-2,5-diazabicyclo[2.2.1]heptan-2-yl)-2-methylbenzoic acid

To a solution of tert-butyl 5-(3-(methoxycarbonyl)-4-methylphenyl)-2,5-diazabicyclo[2.2.1]heptane-2-carboxylate (1030 mg, 2.973 mmol) and LiOH·H₂O (499 mg, 11.9 mmol) in THF (9 mL) was added H₂O (9 mL) at 20° C. The mixture was then stirred at 35° C. for 18 h. After that, the mixture was evaporated in vacuo, adjusted to pH=6-7 with 1N HCl, and then extracted with ethyl acetate (8 mL×3). The combined organic layer was dried over Na₂SO₄ and evaporated in vacuo to give 5-(5-(tert-butoxycarbonyl)-2,5-diazabicyclo[2.2.1]heptan-2-yl)-2-methylbenzoic acid (830 mg, 84%) as a white solid. LCMS (ESI): m/z=333.1, [M+H]+.

### Preparation of P36: 2-(8-(tert-butoxycarbonyl)-3,8-diazabicyclo[3.2.1]octan-3-yl)-5-methylisonicotinic acid

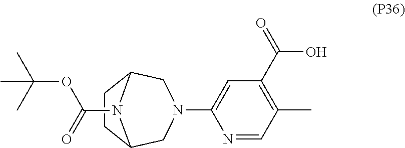 [SMILES: Cc1cnc(N2CC3CCC(C2)N3C(=O)OC(C)(C)C)cc1C(=O)O]

2-(8-(tert-butoxycarbonyl)-3,8-diazabicyclo[3.2.1]octan-3-yl)-5-methylisonicotinic acid was synthesized in a similar manner to 5-(5-(tert-butoxycarbonyl)-2,5-diazabicyclo[2.2.1]heptan-2-yl)-2-methylbenzoic acid using methyl 2-bromo-5-methylisonicotinate and tert-butyl 3,8-diazabicyclo[3.2.1]octane-8-carboxylate.

tert-butyl 3-(4-(methoxycarbonyl)-5-methylpyridin-2-yl)-3,8-diazabicyclo[3.2.1]octane-8-carboxylate. ¹H NMR (400 MHz, CHLOROFORM-d) δ=8.09 (s, 1H), 7.00 (s, 1H), 4.50-4.21 (m, 2H), 4.02-3.75 (m, 5H), 3.23-2.92 (m, 2H), 2.38 (s, 3H), 1.99-1.90 (m, 2H), 1.83-1.73 (m, 2H), 1.52-1.42 (m, 9H). LCMS (ESI): m/z=362.2, [M+H]+.

2-(8-(tert-butoxycarbonyl)-3,8-diazabicyclo[3.2.1]octan-3-yl)-5-methylisonicotinic acid. LCMS (ESI): m/z=348.2, [M+H]+.

### Preparation of P37: 2-(8-(tert-butoxycarbonyl)-3,8-diazabicyclo[3.2.1]octan-3-yl)-5-methylisonicotinic acid

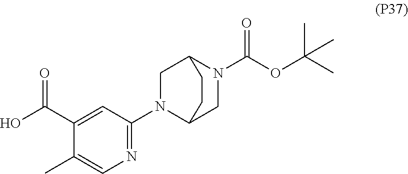 [SMILES: Cc1cnc(N2CC3CCC2CN3C(=O)OC(C)(C)C)cc1C(=O)O]

2-(8-(tert-butoxycarbonyl)-3,8-diazabicyclo[3.2.1]octan-3-yl)-5-methylisonicotinic acid was synthesized in a similar manner to 5-(5-(tert-butoxycarbonyl)-2,5-diazabicyclo[2.2.1]heptan-2-yl)-2-methylbenzoic acid using methyl 2-chloro-5-methylisonicotinate and tert-butyl 2,5-diazabicyclo[2.2.2]octane-2-carboxylate.

tert-butyl 5-(4-(methoxycarbonyl)-5-methylpyridin-2-yl)-2,5-diazabicyclo[2.2.2]octane-2-carboxylate. ¹H NMR (400 MHz, CHLOROFORM-d) δ=8.03 (s, 1H), 6.88-6.74 (m, 1H), 4.85 (br s, 1H), 4.47-4.25 (m, 1H), 3.90 (s, 3H), 3.72-3.39 (m, 4H), 2.36 (s, 3H), 2.15-1.94 (m, 2H), 1.91-1.71 (m, 2H), 1.50-1.42 (m, 9H). LCMS (ESI): m/z=362.2, [M+H]+.

2-(8-(tert-butoxycarbonyl)-3,8-diazabicyclo[3.2.1]octan-3-yl)-5-methylisonicotinic acid. LCMS (ESI): m/z=348.2, [M+H]+.

### Preparation of P38: 4-(1-hydroxy-2-oxo-2-(thiazol-4-ylamino)ethyl)-2-methylbenzoic acid

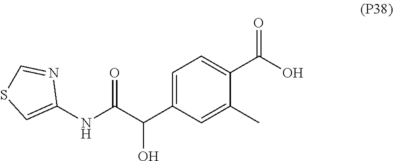 [SMILES: Cc1cc(C(O)C(=O)Nc2cscn2)ccc1C(=O)O]

### Step 1: Preparation of 2-(4-bromo-3-methylphenyl)-2-hydroxyacetonitrile

To a solution of 4-bromo-3-methylbenzaldehyde (5000 mg, 25.12 mmol) in DCM (30 mL) were added TMSCN (3120 mg, 31.4 mmol) and ZnI₂ (401 mg, 1.26 mmol) at 20° C. The mixture was then stirred at 20° C. for 16 h then evaporated in vacuo to give a crude. The crude was dissolved with MeOH (50 mL) and treated with 2 M HCl (15 mL). Then the mixture was stirred at 20° C. for 1 h before evaporated in vacuo to give another crude, which was purified by chromatography (40 g silica gel column, EtOAc/petroleum ether from 0 to 50%) to give 2-(4-bromo-3-methylphenyl)-2-hydroxyacetonitrile (4160 mg, 73.3%) as a yellow oil. ¹H NMR (400 MHz, CHLOROFORM-d) δ=7.60 (d, J=8.4 Hz, 1H), 7.40 (d, J=2.0 Hz, 1H), 7.20 (dd, J=2.2, 8.4 Hz, 1H), 5.48 (br s, 1H), 2.89 (br s, 1H), 2.44 (s, 3H).

### Step 2: Preparation of 2-(4-bromo-3-methylphenyl)-2-hydroxyacetic acid

A solution of 2-(4-bromo-3-methylphenyl)-2-hydroxyacetonitrile (2000.0 mg, 8.847 mmol) in HCl (15 mL) was stirred at 70° C. for 16 h. The reaction mixture was then diluted by H₂O (50 mL) at 20° C. and extracted with ethyl acetate (30 mL×3). The combined organic layer was dried over Na₂SO₄ and evaporated in vacuo to give a crude, which was purified by chromatography (12 g silica gel column, EtOAc/petroleum ether from 0 to 100%) to give 2-(4-bromo-3-methylphenyl)-2-hydroxyacetic acid (700 mg, 32.3%) as a white solid. ¹H NMR (400 MHz, DMSO-d6) δ=12.97-12.48 (m, 1H), 7.55 (d, J=8.2 Hz, 1H), 7.38 (d, J=1.7 Hz, 1H), 7.20-7.15 (m, 1H), 6.12-5.78 (m, 1H), 4.98 (s, 1H), 2.34 (s, 3H).

### Step 3: Preparation of 2-(4-bromo-3-methylphenyl)-2-hydroxy-N-(thiazol-4-yl)acetamide

To a solution of 2-(4-bromo-3-methylphenyl)-2-hydroxyacetic acid (400 mg, 1.63 mmol) in pyridine (16.0 mL) were added EDCI (626 mg, 3.26 mmol) and thiazol-4-amine (471 mg, 2.45 mmol) at 20° C. Then the reaction was stirred at 20° C. for 16 h. After that, the reaction mixture was evaporated in vacuo to give crude, which was purified by chromatography (12 g silica gel column, EtOAc/petroleum ether from 0 to 50%) to give 2-(4-bromo-3-methylphenyl)-2-hydroxy-N-(thiazol-4-yl)acetamide (180 mg, 33.7%) as a white gum. ¹H NMR (400 MHz, DMSO-d6) δ=10.72 (s, 1H), 8.98 (d, J=2.1 Hz, 1H), 7.60 (d, J=2.1 Hz, 1H), 7.55 (d, J=8.2 Hz, 1H), 7.48 (s, 1H), 7.26 (dd, J=1.5, 8.3 Hz, 1H), 6.36 (d, J=5.1 Hz, 1H), 5.18 (d, J=4.9 Hz, 1H), 2.34 (s, 3H). LCMS (ESI): m/z=328.9, [M+H]+.

### Step 4: Preparation of ethyl 4-(1-hydroxy-2-oxo-2-(thiazol-4-ylamino)ethyl)-2-methylbenzoate

To a solution of 2-(4-bromo-3-methylphenyl)-2-hydroxy-N-(thiazol-4-yl)acetamide (180 mg, 0.550 mmol) in EtOH (6.00 mL) were added TEA (167 mg, 1.65 mmol) and PdCl2(dppf) (60.4 mg, 0.0825 mmol) at 20° C. Then the reaction mixture was stirred under CO 50 psi (3.44738 bar) at 80° C. for 16 h before being concentrated in vacuo to give a crude, which was purified by chromatography (12 g silica gel column, EtOAc/petroleum ether from 0 to 50%) to give ethyl 4-(1-hydroxy-2-oxo-2-(thiazol-4-ylamino)ethyl)-2-methylbenzoate (120 mg, 68.1%) as a white gum. LCMS (ESI): m/z=321.1, [M+H]+.

### Step 5: Preparation of 4-(1-hydroxy-2-oxo-2-(thiazol-4-ylamino)ethyl)-2-methylbenzoic acid

To a stirred solution of ethyl 4-(1-hydroxy-2-oxo-2-(thiazol-4-ylamino)ethyl)-2-methylbenzoate (120.0 mg, 0.375 mmol) in THF (1.5 mL) and H₂O (1.5 mL) was added LiOH·H₂O (47.2 mg, 1.12 mmol) at 20° C., then the mixture was stirred at 50° C. for 16 h. After that, the reaction mixture was adjusted to pH=3-5 by 1 N HCl then extracted with ethyl acetate (5 mL×3). The combined organic layer was evaporated in vacuo to give 4-(1-hydroxy-2-oxo-2-(thiazol-4-ylamino)ethyl)-2-methylbenzoic acid (100 mg, 91.3%) as a yellow gum. LCMS (ESI): m/z=293.0, [M+H]+.

### Preparation of P39: 5-(4-(1-aminocyclopropyl)quinolin-2-yl)-N,N-dimethyl-1H-pyrazole-3-carboxamide

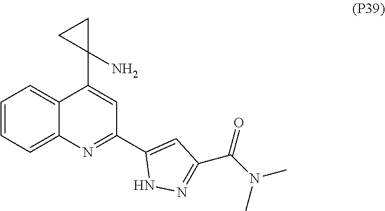 [SMILES: CN(C)C(=O)c1cc(-c2cc(C3(N)CC3)c3ccccc3n2)[nH]n1]

### Step 1: Preparation of 5-(4-(1-((tert-butoxycarbonyl)amino)cyclopropy)quinolin-2-yl)-1-(tetrahydro-2H-pyran-2-yl)-1H-pyrazole-3-carboxylic acid

To a solution of tert-butyl(1-(2-chloroquinolin-4-yl)cyclopropyl)carbamate (2.0 g, 6.274 mmol) and methyl 1-(tetrahydro-2H-pyran-2-yl)-5-(4,4,5,5-tetramethyl-1,3,2-dioxaborolan-2-yl)-1H-pyrazole-3-carboxylate (316 g, 9.41 mmol) in dioxane (25.0 mL) were added Na₂CO₃ (1.99 g, 18.8 mmol) in H₂O (5.0 mL) at 25° C. The mixture was bubbled with N₂ for 1 minute, then Pd(dppf)Cl₂ (459 mg, 0.627 mmol) was added into it. The reaction was purged with N₂ for 1 minute. The resulting mixture was stirred at 100° C. for 16 h under N₂. A black mixture was obtained. The reaction was then diluted with water (50.0 mL) and extracted with EtOAc (50.0 mL×2). The combined organic phase was evaporated and purified by silica gel chromatography (20 g, PE/EA from 100%:0% to 70%:30% then EA/MeOH from 100%:0% to 80%:20%) to give 5-(4-(1-((tert-butoxycarbonyl)amino)cyclopropyl)quinolin-2-yl)-1-(tetrahydro-2H-pyran-2-yl)-1H-pyrazole-3-carboxylic acid (1.9 g, 63.3%) as a black solid. ¹H NMR (400 MHz, METHANOL-d4) δ=8.52 (d, J=7.8 Hz, 1H), 8.13 (d, J=8.3 Hz, 1H), 8.01 (s, 1H), 7.92 (br s, 1H), 7.81 (s, 1H), 7.70 (s, 1H), 7.52-7.42 (m, 1H), 6.71-6.54 (m, 1H), 3.80 (br t, J=11.2 Hz, 2H), 2.62-2.48 (m, 1H), 2.10 (br s, 2H), 1.83-1.68 (m, 2H), 1.61-1.56 (m, 1H), 1.35 (br s, 2H), 1.33 (s, 9H), 1.28 (br s, 2H). LCMS (ESI): m/z=479.2, [M+H]⁺.

### Step 2: Preparation of tert-butyl(1-(2-(3-(dimethylcarbamoyl)-1-(tetrahydro-2H-pyran-2-yl)-1H-pyrazol-5-yl)quinolin-4-yl)cyclopropyl)carbamate

To a mixture of 5-(4-(1-((tert-butoxycarbonyl)amino)cyclopropyl)quinolin-2-yl)-1-(tetrahydro-2H-pyran-2-yl)-1H-pyrazole-3-carboxylic acid (1.9 g, 3.18 mmol) in DMF (15.9 mL) were added DIEA (2.46 g, 19.1 mmol) and HATU (1.81 g, 4.76 mmol) at 0° C., then Me₂NH·HCl (518 mg, 6.35 mmol). The mixture was stirred at 25° C. for 1 h. LCMS showed a peak with desired mass. The reaction was diluted with water (30.0 mL) and extracted with EtOAc (30.0 mL×2). The combined organic layer was washed with brine (30 mL), dried over Na₂SO₄ and evaporated in vacuo to give a crude, which was purified by silica gel chromatography (12 g silica gel column, EtOAc/PE from 0%:100% to 50%:50%) to give a black solid. The black solid was washed with aq. sat. NaHCO₃ (20.0 mL) and extracted with EtOAc (20.0 mL×2). The combined organic layer was evaporated in vacuo to give tert-butyl(1-(2-(3-(dimethylcarbamoyl)-1-(tetrahydro-2H-pyran-2-yl)-1H-pyrazol-5-yl)quinolin-4-yl)cyclopropyl)carbamate (1.8 g, 100%) as a brown solid. LCMS (ESI): m/z=506.3 [M+H]⁺.

### Step 3: Preparation of 5-(4-(1-aminocyclopropyl)quinolin-2-yl)-N,N-dimethyl-1H-pyrazole-3-carboxamide

To solution of tert-butyl(1-(2-(3-(dimethylcarbamoyl)-1-(tetrahydro-2H-pyran-2-yl)-1H-pyrazol-5-yl)quinolin-4-yl)cyclopropyl)carbamate (1.8 g, 3.56 mmol) in CH₂Cl₂ (11.0 mL) was slowly added 4M HCl in 1,4-dioxane (50 mL, 220 mmol) at 0° C. The reaction mixture was then stirred at 25° C. for 20 h. LCMS showed a peak with desired mass. The reaction mixture was evaporated then partitioned between EtOAc and sat. NaHCO₃ (30/30 mL). The organic layer was separated and the aqueous layer was re-extracted with EtOAc (30 mL). The combined organic layer was washed with brine (30 mL), dried and evaporated in vacuo to afford 3-(4-(1-aminocyclopropyl)quinolin-2-yl)-N,N-dimethyl-1H-pyrazole-5-carboxamide (1.1 g, 96%) as a brown solid. LCMS (ESI): m/z=322.0 [M+H]⁺.

### Preparation of P40: 3-(4-(1-aminocyclopropyl)quinolin-2-yl)-N-methyl-1H-pyrazole-5-carboxamide

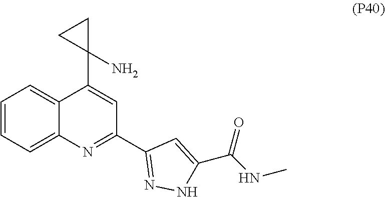 [SMILES: CNC(=O)c1cc(-c2cc(C3(N)CC3)c3ccccc3n2)n[nH]1]

3-(4-(1-aminocyclopropyl)quinolin-2-yl)-N-methyl-1H-pyrazole-5-carboxamide was prepared in a similar manner to P39 using MeNH₂·HCl instead of Me₂NH·HCl. LCMS (ESI): m/z=308.1, [M+H]⁺.

### Preparation of P41: 3-(4-(1-aminocyclopropyl)quinolin-2-yl)-1H-pyrazole-5-carboxamide

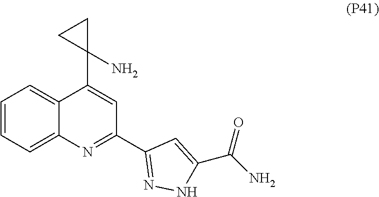 [SMILES: NC(=O)c1cc(-c2cc(C3(N)CC3)c3ccccc3n2)n[nH]1]

3-(4-(1-aminocyclopropyl)quinolin-2-yl)-1H-pyrazole-5-carboxamide was prepared in a similar manner to P39 using NH₄Cl instead of Me₂NH·HCl. LCMS (ESI): m/z=294.1, [M+H]⁺.

### Preparation of P42: 3-(5-(1-aminocyclopropyl)quinolin-7-yl)-N,N-dimethyl-1H-pyrazole-5-carboxamide

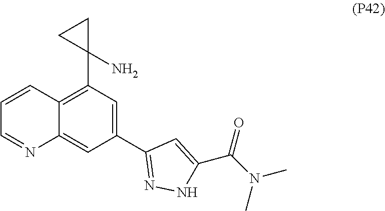 [SMILES: CN(C)C(=O)c1cc(-c2cc(C3(N)CC3)c3cccnc3c2)n[nH]1]

### Step 1: Preparation of 5-(1-((tert-butoxycarbonyl)amino)cyclopropyl)quinolin-7-yl trifluoromethanesulfonate

To a solution of 5-(1-aminocyclopropyl)quinolin-7-yl trifluoromethanesulfonate (1000.0 mg, 2.4 mmol) in CH₂Cl₂ (30.0 mL) were added TEA (974 mg, 9.63 mmol) and (Boc)₂O (1580 mg, 7.22 mmol) at 25° C. The reaction solution was stirred at 40° C. for 16 h. The reaction mixture was then diluted by H₂O (15 mL) and extracted with CH₂Cl₂ (30 mL×3). The organic phase was combined, washed with brine (25 mL), dried with Na₂SO₄, and evaporated in vacuo to give a crude product, which was purified by column chromatography on silica gel (20 g), eluted with EtOAc/PE (0-30%) to give 5-(1-((tert-butoxycarbonyl)amino)cyclopropyl)quinolin-7-yl trifluoromethanesulfonate (700 mg, 67%) as a white solid. LCMS (ESI): m/z=433.1 [M+H]⁺.

### Step 2: Preparation of 5-(5-(1-((tert-butoxycarbonyl)amino)cyclopropyl)quinolin-7-yl)-1-(tetrahydro-2H-pyran-2-yl)-1H-pyrazole-3-carboxylic acid

To a solution of 5-(1-((tert-butoxycarbonyl)amino)cyclopropyl)quinolin-7-yl trifluoromethanesulfonate (700 mg, 1.62 mmol) and (816 mg, 2.43 mmol) in 1,4-dioxane (20 mL) were added Na₂CO₃ (515 mg, 4.86 mmol) and H₂O (5.0 mL) at 25° C. The mixture was purged with N₂ for 1 min then Pd(dppf)Cl₂ (118 mg, 0.162 mmol) was added into it. The mixture was purged with N₂ for 1 min, and the resulting mixture was stirred at 100° C. for 16 h. The reaction solution was filtered and evaporated in vacuo to give a crude product. The crude product was purified via silica gel chromatography (12 g, EtOAc/PE to MeOH/EtOAc, 0-100% to 0-30%) to give 5-(5-(1-((tert-butoxycarbonyl)amino)cyclopropyl)quinolin-7-yl)-1-(tetrahydro-2H-pyran-2-yl)-1H-pyrazole-3-carboxylic acid (900 mg, 100%) as a brown solid. LCMS (ESI): m/z=479.2 [M+H]⁺.

### Step 3: Preparation of tert-butyl(1-(7-(3-(dimethylcarbamoyl-1-(tetrahydro-2H-pyran-2-yl)-1H-pyrazol-5-yl)quinolin-5-yl)cyclopropyl)carbamate

tert-butyl(1-(7-(3-(dimethylcarbamoyl)-1-(tetrahydro-2H-pyran-2-yl)-1H-pyrazol-5-yl)quinolin-5-yl)cyclopropyl)carbamate was prepared using a protocol similar to preparation of P39 step 2 using 5-(5-(1-((tert-butoxycarbonyl)amino)cyclopropyl)quinolin-7-yl)-1-(tetrahydro-2H-pyran-2-yl)-1H-pyrazole-3-carboxylic acid and Me₂NH. LCMS (ESI): m/z=506.2 [M+H]⁺.

### Step 4: Preparation of 3-(5-(1-aminocyclopropyl)quinolin-7-yl)-N,N-dimethyl-1H-pyrazole-5-carboxamide

3-(5-(1-aminocyclopropyl)quinolin-7-yl)-N,N-dimethyl-1H-pyrazole-5-carboxamide was prepared from tert-butyl(1-(7-(3-(dimethylcarbamoyl)-1-(tetrahydro-2H-pyran-2-yl)-1H-pyrazol-5-yl)quinolin-5-yl)cyclopropyl)carbamate using a procedure similar to step 2 of the preparation of P39. ¹H NMR (400 MHz, METHANOL-d4) δ=9.64 (d, J=8.5 Hz, 1H), 9.29 (d, J=4.9 Hz, 1H), 8.80-8.73 (m, 1H), 8.69 (s, 1H), 8.19 (dd, J=5.4, 8.6 Hz, 1H), 7.43 (s, 1H), 3.35 (s, 2H), 3.27 (td, J=1.6, 3.1 Hz, 3H), 3.12 (s, 3H), 1.82-1.76 (m, 2H), 1.56-1.52 (m, 2H). LCMS (ESI): m/z=322.0 [M+H]⁺.

### Preparation of P43: 1-(7-(1-methyl-1H-pyrazol-4-yl)quinolin-5-yl)cyclopropan-1-amine

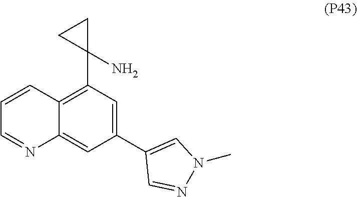 [SMILES: Cn1cc(-c2cc(C3(N)CC3)c3cccnc3c2)cn1]

To a solution of 5-(1-aminocyclopropyl)quinolin-7-yl trifluoromethanesulfonate (100 mg, 0.301 mmol) in 1,4-dioxane (4.0 mL) and H₂O (1.0 mL) were added 1-methyl-4-(4,4,5,5-tetramethyl-1,3,2-dioxaborolan-2-yl)-1H-pyrazole (125 mg, 0.602 mmol), Pd(dppf)Cl₂ (22.0 mg, 0.0301 mmol) and K₃PO₄ (192 mg, 0.903 mmol) at 20° C. The mixture was bubbled with N₂ for 1 min and stirred at 100° C. for 2 h. It was then combined with the reaction of another batch of 5-(1-aminocyclopropyl)quinolin-7-yl trifluoromethanesulfonate (30 mg, 0.090 mmol) for workup. The mixture was evaporated in vacuo to give a crude, which was purified by chromatography (20 g silica gel column, MeOH/EtOAc from 0 to 30%) to give 1-(7-(1-methyl-1H-pyrazol-4-yl)quinolin-5-yl)cyclopropan-1-amine (52 mg, 50.0%) as a brown gum. LCMS (ESI): m/z=265.2 [M+H]⁺.

### Preparation of P44: 2-methyl-4-((oxazol-4-ylmethoxy)methyl)benzoic acid

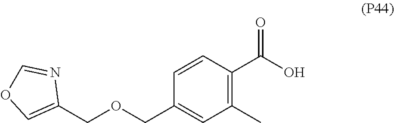 [SMILES: Cc1cc(COCc2cocn2)ccc1C(=O)O]

2-methyl-4-((oxazol-4-ylmethoxy)methyl)benzoic acid was prepared using a similar procedure in step 3-4 of Example 274 using methyl 4-(chloromethyl)-2-methylbenzoate and oxazol-4-ylmethanol.

methyl 2-methyl-4-((oxazol-4-ylmethoxy)methyl)benzoate: ¹H NMR (400 MHz, CDCl₃) δ=8.03-7.82 (m, 2H), 7.68 (s, 1H), 7.28 (s, 1H), 7.24 (s, 1H), 4.64 (s, 2H), 4.54 (s, 2H), 3.90 (s, 3H), 2.62 (s, 3H). LCMS (ESI): m/z=262.1 [M+H]⁺.
2-methyl-4-((oxazol-4-ylmethoxy)methyl)benzoic acid: LCMS (ESI): m/z=248.1 [M+H]⁺.

### EXAMPLES

### Example 1: N¹-(1-(3-(1H-1,2,3-triazol-1-yl)naphthalen-1-yl)ethyl)-6-methylisophthalamide

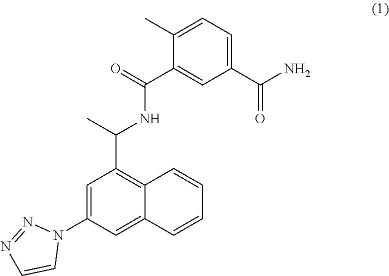 [SMILES: Cc1ccc(C(N)=O)cc1C(=O)NC(C)c1cc(-n2ccnn2)cc2ccccc12]

### Step 1. Preparation of N-(1-(3-bromonaphthalen-1-yl)ethyl)-5-cyano-2-methylbenzamide

To a mixture of 1-(3-bromonaphthalen-1-yl)ethan-1-amine (243.0 mg, 0.971 mmol), DIEA (377 mg, 2.91 mmol) and 5-cyano-2-methylbenzoic acid (157 mg, 0.971 mmol) in DMF (4.86 mL) was added HATU (554 mg, 1.46 mmol) in batches. The reaction mixture was stirred at 20° C. for 12 h. LCMS showed the main peak was of the desired product. Solvent was then evaporated. The mixture was then extracted with ethyl acetate (3 mL×2) after adding H₂O (4 mL) to it. The organic phase was purified with silica gel column chromatography (eluted with EtOAc in PE from 0% to 30%) to give N-(1-(3-bromonaphthalen-1-yl)ethyl)-5-cyano-2-methylbenzamide (360 mg, 94.2%) as a colorless oil. LCMS (ESI): m/z=395.0 [M+H]⁺.

### Step 2. Preparation of N¹-(1-(3-(1H-1,2,3-triazol-1-yl)naphthalen-1-yl)ethyl)-6-methylisophthalamide

To a solution of N-(1-(3-bromonaphthalen-1-yl)ethyl)-5-cyano-2-methylbenzamide (360.0 mg, 0.915 mmol) in DMSO (10.0 mL) and H₂O (2.0 mL) were added K₃PO₄ (389 mg, 1.83 mmol), (+/−)-trans-N,N′dimethyl cyclohexane-1,2-diamine (26.0 mg, 0.183 mmol) and CuI (69.7 mg, 0.366 mmol) at 25° C. It was stirred at 130° C. for 16 h. LCMS showed two products with a desired mass (28% and 17%). The mixture was then purified with silica gel column chromatography (eluted with EtOAc in PE from 0% to 80%) to give crude-1 (80 mg) and crude-2 (30 mg) as yellow solids. The crude-2 was dissolved in DMF (0.5 mL) and purified by prep-HPLC to afford N¹-(1-(3-(1H-1,2,3-triazol-1-yl)naphthalen-1-yl)ethyl)-6-methylisophthalamide (3.7 mg). ¹H NMR (400 MHz, DMSO-dc) δ=9.16-9.06 (m, 1H), 9.10 (d, J=7.7 Hz, 1H), 8.93 (d, J=0.9 Hz, 1H), 9.01-8.85 (m, 1H), 8.40 (d, J=1.8 Hz, 1H), 8.35 (br d, J=7.3 Hz, 1H), 824 (d, J=2.0 Hz, 1H), 8.14-8.07 (m, 1H), 8.03 (s, 1H), 7.98 (br s, 1H), 7.87 (d, J=1.8 Hz, 1H), 7.83 (dd, J=1.8, 7.9 Hz, 1H), 7.73-7.65 (m, 2H), 7.37 (br s, 1H), 7.31 (d, J=7.9 Hz, 1H), 6.01 (t, J=7.3 Hz, 1H), 230 (s, 3H), 1.66 (d, J=6.8 Hz, 3H). LCMS (ESI): m/z=400.2 [M+H].

### Prep-HPLC Condition:

Column: Boston Prime C18 150*30 mm*5 μm;
Mobile phase: water (ammonium hydroxide v/v)-acetonitrile; B %: 30%-50%,
Flow Rate (mL/min): 35 mL/min.

### Example 2: N¹-(1-(3-(2H-1,2,3-triazol-2-yl)naphthalen-1-yl)ethyl)-6-methylisophthalamide

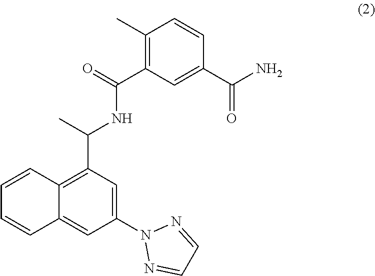 [SMILES: Cc1ccc(C(N)=O)cc1C(=O)NC(C)c1cc(-n2nccn2)cc2ccccc12]

Crude-1 (prepared in Step 2 of Example 1) was dissolved in DMF (0.5 mL) and purified by prep-HPLC to afford N¹-(1-(3-(2H-1,2,3-triazol-2-yl)naphthalen-1-yl)ethyl)-6-methylisophthalamide (40.45 mg) as a white solid after lyophilization. ¹H NMR (400 MHz, DMSO-de) δ=9.18 (d, J=7.9 Hz, 1H), 8.49 (d, J=1.8 Hz, 1H), 8.40 (d, J=2.0 Hz, 1H), 8.36-8.29 (m, 1H), 8.22-8.13 (m, 3H), 7.97 (br s, 1H), 7.88 (d, J=1.5 Hz, 1H), 7.83 (dd, J=1.8, 7.9 Hz, 1H), 7.71-7.61 (m, 2H), 7.38 (br s, 1H), 7.32 (d, J=7.9 Hz, 1H), 5.99 (br t, J=7.3 Hz, 1H), 2.31 (s, 3H), 1.65 (d, J=6.8 Hz, 3H). LCMS (ESI): m/z=400.2 [M+H]⁺.

### Prep-HPLC Condition:

Column: Boston Prime C18 150*30 mm*5 μm;
Mobile phase: water (ammonium hydroxide v/v)-acetonitrile; B %: 38%-58%,
Flow Rate (mL/min): 35 mL/min.

### Example 3: 5-(aminomethyl)-2-methyl-N-(1-(3-(1-methyl-1H-pyrazol-4-yl)naphthalen-1-yl)ethyl)benzamide

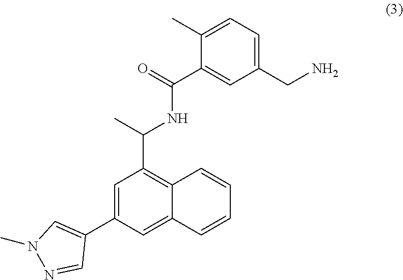 [SMILES: Cc1ccc(CN)cc1C(=O)NC(C)c1cc(-c2cnn(C)c2)cc2ccccc12]

### Step 1. Preparation of 5-cyano-2-methyl-N-(1-(3-(1-methyl-1H-pyrazol-4-yl)naphthalen-1-yl)ethyl)benzamide

To a mixture of N-(1-(3-bromonaphthalen-1-yl)ethyl)-5-cyano-2-methylbenzamide (50.0 mg, 0.13 mmol) and 1-methyl-4-(4,4,5,5-tetramethyl-1,3,2-dioxaborolan-2-yl)-1H-pyrazole (26.5 mg, 0.127 mmol) in 1,4-dioxane (1.0 mL) and H₂O (0.2 mL) were added K₃PO₄ (67.5 mg, 0.318 mmol) and Pd(dppf)Cl₂ (9.30 mg, 0.0127 mmol). The mixture was degassed with N₂ three times, heated to 80° C., and then stirred at 80° C. for 16 h. LCMS showed the desired product was formed. The mixture was purified with silica gel column chromatography (with PE:EtOAc=0%:60%) to give 5-cyano-2-methyl-N-(1-(3-(1-methyl-1H-pyrazol-4-yl)naphthalen-1-yl)ethyl)benzamide (50 mg, 100%) as a yellow solid. LCMS (ESI): m/z=395.2 [M+H]⁺.

### Step 2. Preparation of 5-(aminomethyl)-2-methyl-N-(1-(3-(1-methyl-1H-pyrazol-4-yl)naphthalen-1-yl)ethyl)benzamide

To a solution of Raney-Ni (30.0 mg, 0.35 mmol) in THF (3.0 mL) was added 5-cyano-2-methyl-N-(1-(3-(1-methyl-1H-pyrazol-4-yl)naphthalen-1-yl)ethyl)benzamide (50.0 mg, 0.13 mmol) in IPA (3.0 mL) and NH₃·H₂O (0.5 mL). The mixture was degassed and refilled with H₂ for three times, stirred at 20° C. and 20 psi (1.37895 bar) for 16 h. LCMS showed the desired product was formed. The mixture was purified by prep-HPLC to afford 5-(aminomethyl)-2-methyl-N-(1-(3-(1-methyl-1H-pyrazol-4-yl)naphthalen-1-yl)ethyl)benzamide (6.59 mg, 13%) as a white solid after lyophilization. ¹H NMR (400 MHz, MeOD-d₄) δ=8.25-8.19 (m, 1H), 8.09 (s, 1H), 7.97 (s, 1H), 7.95 (s, 1H), 7.92-7.87 (m, 1H), 7.84 (d, J=1.3 Hz, 1H), 7.56-7.47 (m, 2H), 7.32-7.28 (m, 2H), 7.23-7.18 (m, 1H), 6.07 (d, J=6.8 Hz, 1H), 3.96 (s, 3H), 3.77 (s, 2H), 2.33 (s, 3H), 1.75 (d, J=6.8 Hz, 3H). LCMS (ESI): m/z=399.3 [M+H]⁺.

### Prep-HPLC Condition:

Column: Boston Prime C18 150*30 mm*5 μm;
Mobile phase: water (ammonium hydroxide v/v)-acetonitrile; B %: 35%-55%,
Flow Rate (mL/min): 35 mL/min.

### Example 4: (2R)—N-(4-methyl-3-((1-(3-(1-methyl-1H-pyrazol-4-yl)naphthalen-1-yl)ethyl)carbamoyl)phenyl)piperidine-2-carboxamide

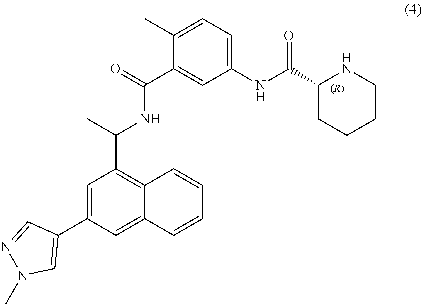

### Step 1. Preparation of tert-butyl 2R)-2-((4-methyl-3-((1-(3-(1-methyl-1H-pyrazol-4-yl)naphthalen-1-yl)ethyl)carbamoyl)phenyl)carbamoyl)piperidine-1-carboxylate

To a mixture of 1-(3-(1-methyl-1H-pyrazol-4-yl)naphthalen-1-yl)ethan-1-amine (P5) (12.0 mg, 0.048 mmol) in DMF (0.239 mL) were added DIEA (18.5 mg, 0.143 mmol) and (R)-5-(1-(tert-butoxycarbonyl)piperidine-2-carboxamido)-2-methylbenzoic acid (17.3 mg, 0.0477 mmol) and then was added HATU (27.2 mg, 0.0716 mmol) in aliquots. The reaction mixture was stirred at 20° C. for 12 h. LCMS showed the desired product was formed. The mixture was treated with H₂O (2 mL) and extracted with ethyl acetate (1 mL×2), the organic phase was then purified with silica gel column chromatography (eluting with ethyl acetate in PE from 0 to 50%) to give tert-butyl(2R)-2-((4-methyl-3-((1-(3-(1-methyl-1H-pyrazol-4-yl)naphthalen-1-yl)ethyl)carbamoyl)phenyl)carbamoyl)piperidine-1-carboxylate (28 mg, 98%) as a colorless oil. It was used in the next step directly. LCMS (ESI): m/z=596.3 [M+H]⁺.

### Step 2. Preparation of (2R)—N-(4-methyl-3-((1-(3-(1-methyl-1H-pyrazol-4-yl)naphthalen-1-yl)ethyl)carbamoyl)phenyl)piperidine-2-carboxamide

To a solution of tert-butyl(2R)-2-((4-methyl-3-((1-(3-(1-methyl-1H-pyrazol-4-yl)naphthalen-1-yl)ethyl)carbamoyl)phenyl)carbamoyl)piperidine-1-carboxylate (28.0 mg, 0.047 mmol) in dichloromethane (1.0 mL) was added 4M HCl/dioxane (1.0 mL) at 25° C. After addition, the solution was stirred at room temperature (25° C.) for 2 h. LCMS showed the desired product was formed. The reaction mixture was concentrated in vacuo then purified by prep-HPLC to afford (2R)—N-(4-methyl-3-((1-(3-(1-methyl-1H-pyrazol-4-yl)naphthalen-1-yl)ethyl)carbamoyl)phenyl)piperidine-2-carboxamide (3.04 mg, 13%) as a white solid after lyophilization. ¹H NMR (400 MHz, DMSO-d₆) δ=9.71 (br d, J=5.9 Hz, 1H), 9.00-8.83 (m, 1H), 8.27 (d, J=3.5 Hz, 1H), 8.18 (br dd, J=2.8, 6.5 Hz, 1H), 8.01 (s, 1H), 7.98 (s, 1H), 7.91 (dd, J=3.0, 6.7 Hz, 1H), 7.85 (s, 1H), 7.80 (dd, J=2.0, 6.6 Hz, 1H), 7.56-7.50 (m, 3H), 7.15 (d, J=8.4 Hz, 1H), 5.92 (br t, J=7.0 Hz, 1H), 3.93 (s, 3H), 324 (br d, J=6.8 Hz, 1H), 3.01-2.93 (m, 1H), 2.22 (s, 3H), 1.84-1.75 (m, 2H), 1.60 (d, J=6.8 Hz, 3H), 1.54-1.24 (m, 5H). LCMS (ESI): m/z=496.4 [M+H]⁺.

### Prep-HPLC Condition:

Column: Boston Prime C18 150*30 mm*5 μm;
Mobile phase: water (ammonium hydroxide v/v)-acetonitrile; B %: 38%-58%,
Flow Rate (mL/min): 35 mL/min.

The following examples were prepared in a similar manner to Example 4 using an appropriate amine and an appropriate carboxylic acid.

| Ex. | Structure; | Analogous method, | |
|---|---|---|---|
| # | Compound Name | reactants | Characterizing Data |
| 5 | 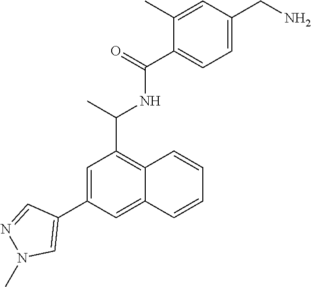 [SMILES: Cc1cc(CN)ccc1C(=O)NC(C)c1cc(-c2cnn(C)c2)cc2ccccc12] | Example 4, Step 1, using 1- (3-(1-methyl-1H-pyrazol-4- yl)naphthalen-1-yl)ethan-1- amine (P5) and 4-(((tert- butoxycarbonyl)amino) methyl)-2-methylbenzoic acid; then Example 4, Step 2 | ¹H NMR (400 MHz, CDCl₃) δ = 8.21-8.16 (m, 1H), 7.89-7.83 (m, 3H), 7.74 (s, 1H), 7.67 (s, 1H), 7.54- 7.49 (m, 2H), 7.14 (s, 1H), 7.08 (d, J = 7.3 Hz, 1H), 6.15-6.09 (m, 1H), 6.00 (br d, J = 7.9 Hz, 1H), 3.99 (s, 3H), 3.81 (s, 2H), 2.45 (s, 3H), 1.83 (d, J = 6.6 Hz, 3H). LCMS (ESI): m/z = 399.3 [M + H]⁺. |
| | 4-(aminomethyl)-2-methyl- | | |
| | N-(1-(3-(1-methyl-1H- | | |
| | pyrazol-4-yl)naphthalen-1- | | |
| | yl)ethyl)benzamide | | |
| 10 | 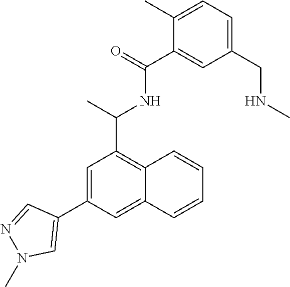 [SMILES: CNCc1ccc(C)c(C(=O)NC(C)c2cc(-c3cnn(C)c3)cc3ccccc23)c1] | Example 4, Step 1, using 1- (3-(1-methyl-1H-pyrazol-4- yl)naphthalen-1-yl)ethan-1- amine (P5) and 5-(((tert- butoxycarbonyl)(methyl)ami no)methyl)-2-methylbenzoic acid (P9); then Example 4, Step 2 | ¹H NMR (400 MHz, DMSO-d6) δ = 8.85 (d, J = 8.1 Hz, 1H), 8.22 (s, 1H), 8.21-8.17 (m, 1H), 8.02-7.86 (m, 4H), 7.55- 7.49 (m, 2H), 7.31-7.23 (m, 2H), 7.21-7.14 (m, 1H), 5.94 (quin, J = 7.2 Hz, 1H), 3.92 (s, 3H), 3.65- 3.44 (m, 2H), 2.26 (d, J = 4.3 Hz, 6H), 1.62 (d, J = 6.9 Hz, 3H). LCMS m/z = 413.3, [M + H]⁺. |
| | 2-methyl-N-(1-(3-(1- | | |
| | methyl-1H-pyrazol-4- | | |
| | yl)naphthalen-1-yl)ethyl)- | | |
| | 5-((methylamino)methyl) | | |
| | benzamide | | |
| 13 | 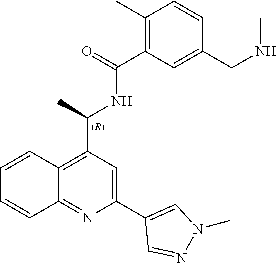 | Example 4, Step 1, using (R)-1-(2-(1-methyl-1H- pyrazol-4-yl)quinolin-4- yl)ethan-1-amine (prepared in Example 11, Step 2) and 5-(((tert-butoxycarbonyl) (methyl)amino)methyl)-2- methylbenzoic acid (P9); then Example 4, Step 2 | ¹H NMR (400 MHz, MeOD) δ ppm 8.35 (s, 1H), 8.30 (d, J = 8.6 Hz, 1H), 8.19 (s, 1H), 8.08 (d, J = 8.4 Hz, 1H), 7.88 (s, 1H), 7.78 (t, J = 7.7 Hz, 1H), 7.64 (t, J = 7.3 Hz, 1H), 7.38-7.31 (m, 2H), 7.28-7.23 (m, 1H), 6.06 (q, J = 6.9 Hz, 1H), 4.02 (s, 3H), 3.72 (s, 2H), 2.40 (s, 3H), 2.35 (s, 3H), 1.76 (d, J = 6.9 Hz, 3H). LCMS (ESI): m/z = 414 [M + H]⁺. |
| | (R)-2-methyl-N-(1-(2-(1- | | |
| | methyl-1H-pyrazol-4- | | |
| | yl)quinolin-4-yl)ethyl)-5- | | |
| | ((methylamino)methyl) | | |
| | benzamide | | |
| 14 | 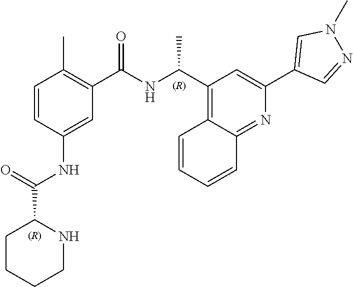 | Example 4, Step 1, using (R)-1-(2-(1-methyl-1H- pyrazol-4-yl)quinolin-4- yl)ethan-1-amine (prepared in Example 11, Step 2) and (R)-5-(1-(tert- butoxycarbonyl) piperidine- 2-carboxamido)-2- methylbenzoic acid; then Example 4, Step 2 | ¹H NMR (400 MHz, MeOD) δ ppm 9.73 (br s, 1H), 8.99 (br d, J = 7.8 Hz, 1H), 8.58-8.37 (m, 1H), 8.30-8.17 (m, 1H), 8.12 (s, 1H), 7.98 (d, J = 8.5 Hz, 1H), 7.90-7.82 (m, 2H), 7.74 (t, J = 7.6 Hz, 1H), 7.66-7.51 (m, 2H), 7.17 (d, J = 8.4 Hz, 1H), 5.88 (quin, J = 6.9 Hz, 1H), 3.96 (s, 3H), 3.30-3.21 (m, 2H), 2.97 (br d, J = 13.1 Hz, 1H), 2.22 (s, 3H), 1.80 (br s, 2H), 1.60 (d, J = 6.9 Hz, 3H), 1.54-1.35 (m, 4H). LCMS (ESI): m/z = 497 [M + H]⁺. |
| | (R)-N-(4-methyl-3-(((R)-1- | | |
| | (2-(1-methyl-1H-pyrazol-4- | | |
| | yl)quinolin-4-yl)ethyl) | | |
| | carbamoyl)phenyl)piperidine- | | |
| | 2-carboxamide | | |
| 55 | 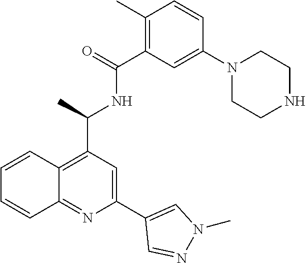 [SMILES: Cc1ccc(N2CCNCC2)cc1C(=O)N[C@H](C)c1cc(-c2cnn(C)c2)nc2ccccc12] | Example 4, Step 1, using (R)-1-(2-(1-methyl-1H- pyrazol-4-yl)quinolin-4- yl)ethan-1-amine (prepared in Example 11, Step 2) and 5-(4-(tert-butoxycarbonyl) piperazin-1-yl)-2- methylbenzoic acid; then Example 4, Step 2 | ¹H NMR (400 MHz, MeOD) δ ppm 8.34 (s, 1H), 8.29 (d, J = 8.4 Hz, 1H), 8.17 (s, 1H), 8.08 (d, J = 8.3 Hz, 1H), 7.91 − 7.85 (m, 1H), 7.79 (t, J = 7.6 Hz, 1H), 7.63 (t, J = 7.6 Hz, 1H), 7.15 (d, J = 8.4 Hz, 1H), 7.00 (d, J = 8.8 Hz, 1H), 6.96 (s, 1H), 6.02 (q, J = 6.8 Hz, 1H), 4.02 (s, 3H), 3.30 − 3.09 (m, 4H), 3.07 − 2.92 (m, 4H), 2.26 (s, 3H), 1.74 (d, J = 7.0 Hz, 3H). LCMS (ESI): m/z = 455.3, [M + H]⁺. |
| | (R)-2-methyl-N-(1-(2-(1- | | |
| | methyl-1H-pyrazol-4- | | |
| | yl)quinolin-4-yl)ethyl)-5- | | |
| | (piperazin-1-yl)benzamide | | |
| 57 | 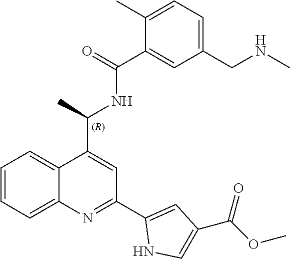 | Example 4, Step 1, methyl (R)-5-(4-(1- aminoethyl)quinolin-2-yl)- 1H-pyrrole-3-carboxylate and 5-(((tert- butoxycarbonyl)(methyl) amino)methyl)-2- methylbenzoic acid (P9); then Example 4, Step 1 | ¹H NMR (400 MHz, MeOD-d₄) δ = 8.58-8.54 (m, 1H), 8.26 (d, J = 7.9 Hz, 1H), 8.09 (d, J = 8.1 Hz, 1H), 7.95 (s, 1H), 7.77 (t, J = 7.2 Hz, 1H), 7.66- 7.58 (m, 2H), 7.40 (s, 1H), 7.37 (d, J = 8.1 Hz, 1H), 7.32 (d, J = 1.5 Hz, 1H), 7.30 (d, J = 7.7 Hz, 1H), 6.05 (q, J = 6.9 Hz, 1H), 3.85 (s, 5H), 2.48 (s, 3H), 2.38 (s, 3H), 1.74 (d, J = 7.0 Hz, 3H). LCMS (ESI): m/z = 457.4 [M + H]⁺. |
| | methyl (R)-5-(4-(1-(2- | | |
| | methyl-5-((methylamino) | | |
| | methyl)benzamido)ethyl) | | |
| | quinolin-2-yl)-1H-pyrrole- | | |
| | 3-carboxylate | | |
| 59 | 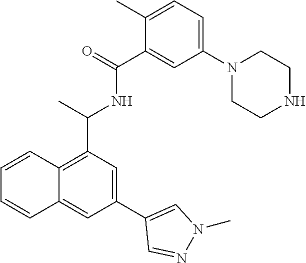 [SMILES: Cc1ccc(N2CCNCC2)cc1C(=O)NC(C)c1cc(-c2cnn(C)c2)cc2ccccc12] | Example 4, Step 1, 1-(3-(1- methyl-1H-pyrazol-4- yl)naphthalen-1-yl)ethan-1- amine (P5) and 5-(4-(tert- butoxycarbonyl) piperazin- 1-yl)-2-methylbenzoic acid; then Example 4, Step 2, tert-butyl 4-(4-methyl-3-((1- (3-(1-methyl-1H-pyrazol-4- yl)naphthalen-1- yl)ethyl)carbamoyl) phenyl)piperazine-1- carboxylate | ¹H NMR (400 MHz, MeOD-d₄) δ = 8.22 (br d, J = 9.0 Hz, 1H), 8.09 (s, 1H), 7.97 (d, J = 7.1 Hz, 2H), 7.94-7.89 (m, 1H), 7.86 (s, 1H), 7.57-7.49 (m, 2H), 7.12 (d, J = 8.4 Hz, 1H), 6.96 (dd, J = 2.6, 8.4 Hz, 1H), 6.92 (d, J = 2.6 Hz, 1H), 6.06 (q, J = 6.9 Hz, 1H), 3.98 (s, 3H), 3.09 (dd, J = 3.9, 6.2 Hz, 4H), 3.02-2.92 (m, 4H), 2.27 (s, 3H), 1.75 (d, J = 7.0 Hz, 3H). LCMS (ESI): m/z = 454.4 [M + H]⁺. |
| | 2-methyl-N-(1-(3-(1- | | |
| | methyl-1H-pyrazol-4- | | |
| | yl)naphthalen-1-yl)ethyl)- | | |
| | 5-(piperazin-1- | | |
| | yl)benzamide | | |
| 60 | 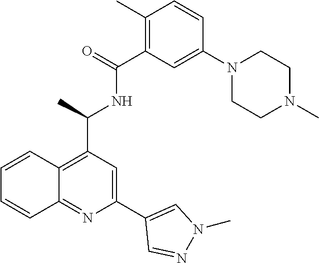 [SMILES: Cc1ccc(N2CCN(C)CC2)cc1C(=O)N[C@H](C)c1cc(-c2cnn(C)c2)nc2ccccc12] | Example 4, Step 1, (R)-1- (2-(1-methyl-1H-pyrazol-4- yl)quinolin-4-yl)ethan-1- amine (prepared in Example 11, Step 1) and 2- methyl-5-(4- methylpiperazin-1-yl) benzoic acid | ¹H NMR (400 MHz, MeOD) δ ppm 8.34 (s, 1H), 8.28 (d, J = 7.8 Hz, 1H), 8.18 (s, 1H), 8.08 (d, J = 8.0 Hz, 1H), 7.88 (s, 1H), 7.78 (t, J = 7.5 Hz, 1H), 7.70-7.56 (m, 1H), 7.15 (d, J = 7.8 Hz, 1H), 7.02-6.93 (m, 2H), 6.02 (q, J = 6.9 Hz, 1H), 4.19 (s, 1H), 4.02 (s, 3H), 3.22- 3.14 (m, 4H), 2.68-2.57 (m, 4H), 2.36 (s, 3H), 2.26 (s, 3H), 1.74 (d, J = 7.0 Hz, 3H). LCMS (ESI): m/z = 469.5 [M+ H]⁺. |
| | (R)-2-methyl-N-(1-(2-(1- | | |
| | methyl-1H-pyrazol-4- | | |
| | yl)quinolin-4-yl)ethyl)-5-(4- | | |
| | methylpiperazin-1- | | |
| | yl)benzamide | | |
| 65 | 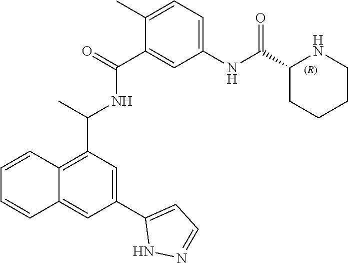 | Example 4, Step 1, using P11 and (R)-5-(1-(tert- butoxycarbonyl) piperidine- 2-carboxamido)-2-methyl benzoic acid; then Example 4, Step 2 | ¹H NMR (400 MHz, CD₃OD, 296 K) δ (ppm) = 8.39 − 8.30 (m, 2H), 8.23 (t, J = 2.1 Hz, 1H), 8.10 − 8.02 (m, 2H), 7.74 − 7.61 (m, 3H), 7.57 − 7.49 (m, 1H), 7.26 − 7.19 (m, 2H), 6.16 − 6.04 (m, 1H), 3.98 − 3.90 (m, 1H), 3.46 (br d, J = 12.5 Hz, 1H), 3.16 − 3.03 (m, 1H), 2.33 − 2.27 (m, 4H), 2.03 − 1.88 (m, 2H), 1.84 − 1.78 (m, 3H), 1.76 − 1.72 (m, 1H), 1.76 − 1.60 (m, 3H). LCMS (ESI): m/z = 482.4, [M + H]⁺. |
| | (2R)-N-(3-((1-(3-(1H- | | |
| | pyrazol-5-yl)naphthalen-1- | | |
| | yl)ethyl)carbamoyl)-4- | | |
| | methylphenyl)piperidine-2- | | |
| | carboxamide | | |
| 73 | 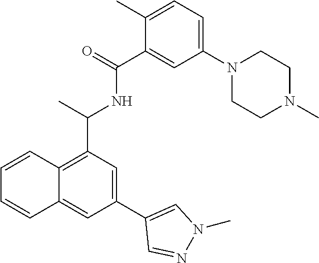 [SMILES: Cc1ccc(N2CCN(C)CC2)cc1C(=O)NC(C)c1cc(-c2cnn(C)c2)cc2ccccc12] | Example 4, Step 1, using 1- (3-(1-methyl-1H-pyrazol-4- yl)naphthalen-1-yl)ethan-1- amine (P5) and 2-methyl-5- (4-methylpiperazin-1-yl) benzoic acid | ¹H NMR (400 MHz, METHANOL-d₄) δ = 8.22 (br d, J = 7.3 Hz, 1H), 8.09 (s, 1H), 7.99 − 7.95 (m, 2H), 7.91 (d, J = 6.9 Hz, 1H), 7.85 (s, 1H), 7.56 − 7.49 (m, 2H), 7.12 (d, J = 8.4 Hz, 1H), 6.97 (dd, J = 2.6, 8.6 Hz, 1H), 6.92 (d, J = 2.4 Hz, 1H), 6.06 (q, J = 6.7 Hz, 1H), 4.01 − 3.96 (m, 3H), 3.20 − 3.12 (m, 4H), 2.65 − 2.56 (m, 4H), 2.36 (s, 3H), 2.26 (s, 3H), 1.75 (d, J = 7.0 Hz, 3H). LCMS (ESI): m/z = 468.5, [M + H]⁺. |
| | 2-methyl-N-(1-(3-(1- | | |

methyl-1H-pyrazol-4-
yl)naphthalen-1-yl)ethyl)-
5-(4-methylpiperazin-1-
yl)benzamide

### Example 6: 4-((((1H-imidazol-2-yl)methyl)amino)methyl)-2-methyl-N-(1-(3-(1-methyl-1H-pyrazol-4-yl)naphthalen-1-yl)ethyl)benzamide

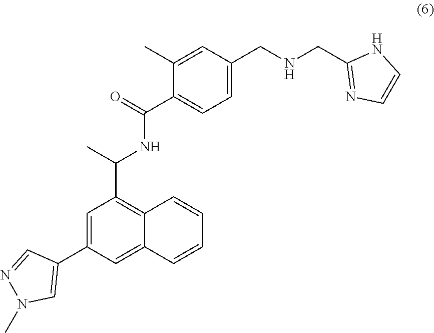 [SMILES: Cc1cc(CNCc2ncc[nH]2)ccc1C(=O)NC(C)c1cc(-c2cnn(C)c2)cc2ccccc12]

To a solution of 4-(aminomethyl)-2-methyl-N-(1-(3-(1-methyl-1H-pyrazol-4-yl)naphthalen-1-yl)ethyl)benzamide (50.0 mg, 0.13 mmol) in 1,2-dichloroethane (1.25 mL) were added 1H-imidazole-2-carbaldehyde (12.1 mg, 0.125 mmol) and AcOH (13.8 mg, 0.314 mmol) at 15° C. The mixture was stirred at 50° C. for 4 h, then NaBH(OAC)₃ (53.2 mg 0.251 mmol) was added to the mixture. The resulting mixture was stirred at 15° C. for 16 h before being concentrated in vacuo and purified by prep-HPLC to afford 4-((((1H-imidazol-2-yl)methyl)amino)methyl)-2-methyl-N-(1-(3-(1-methy-1H-pyrazol-4-yl)naphthalen-1-yl)ethyl)benzamide (9.75 mg, 16%) as a white solid after lyophilization. ¹H NMR (400 MHz, DMSO-de) 6:=10.84-10.16 (m, 4H), 9.00 (d, J=8.1 Hz, H), 8.24 (s, 1H), 8.17 (br dd, J=3.5, 6.2 Hz, H), 8.01 (s, 1H), 7.96 (s, 1H), 793-7.84 (m, 1H), 7.74 (s, 2H), 7.60 (s, 1H), 7.54-7.49 (m, 2H) 7.45 (br d, J=2.9 Hz, 2H), 7.40-7.35 (m, 1H), 5.99-5.86 (m, 1H), 4.54 (s, 2H) 4.31 (s, 2H), 3.91 (s, 3H), 2.29 (s, 3H), 1.62 (d, J=6.9 Hz, 3H). LCMS (ESI): m/z=479.4 [M+H]⁺.

### Prep-HPLC Condition:

Column: Boston Prime C18 150*30 mm*5 μm;
Mobile phase: water (ammonium hydroxide v/v)-acetonitrile; B %: 8%-48%,
Flow Rate (mL/min): 30 mL/min.

The following examples were prepared in a similar manner to Example 6 using an appropriate amine and an appropriate aldehyde.

| Ex. | Structure; | Analogous method, | |
|---|---|---|---|
| # | Compound Name | reactants | Characterizing Data |
| 7 | 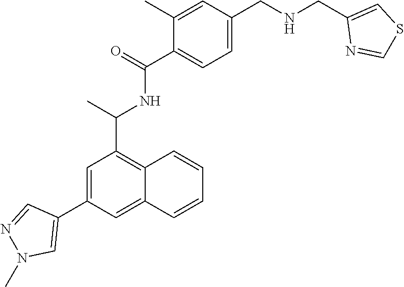 [SMILES: Cc1cc(CNCc2cscn2)ccc1C(=O)NC(C)c1cc(-c2cnn(C)c2)cc2ccccc12] | Example 6, using 4- (aminomethyl)-2-methyl-N- (1-(3-(1-methyl-1H-pyrazol- 4-yl)naphthalen-1- yl)ethyl)benzamide and thiazole-4- carbaldehyde | ¹H NMR (400 MHz, DMSO-d₆) δ = 9.77 (br s, 2H), 9.22 (d, J = 2.0 Hz, 1H), 8.96 (d, J = 8.1 Hz, 1H), 8.23 (s, 1H), 8.19 − 8.15 (m, 1H), 8.01 (s, 1H), 7.95 (d, J = 0.7 Hz, 1H), 7.93 (d, J = 1.8 Hz, 1H), 7.92-7.88 (m, 1H), 7.87 (d, J = 1.5 Hz, 1H), 7.54-7.49 (m, 2H), 7.42- 7.35 (m, 3H), 5.93 (t, J = 7.6 Hz, 1H), 4.30-4.14 (m, 4H), 3.91 (s, 3H), 2.30 (s, 3H), 1.63 (d, J = 7.0 Hz, 3H). LCMS (ESI): m/z = 496.4 [M + H]⁺. |
| | 2-methyl-N-(1-(3-(1-methyl- | | |
| | 1H-pyrazol-4-yl)naphthalen- | | |
| | 1-yl)ethyl)-4-(((thiazol-4- | | |
| | ylmethyl)amino)methyl) | | |
| | benzamide | | |
| 74 | 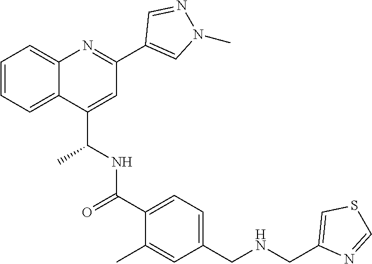 [SMILES: Cc1cc(CNCc2cscn2)ccc1C(=O)N[C@H](C)c1cc(-c2cnn(C)c2)nc2ccccc12] (R)-2-methyl-N-(1-(2-(1- methyl-1H-pyrazol-4- | Example 6, thiazole-4- carbaldehyde and (R)-4- (aminomethyl)-2-methyl-N- (1-(2-(1-methyl-1H-pyrazol- 4-yl)quinolin-4- yl)ethyl)benzamide (prepared from (R)-1-(2-(1- methyl-1H-pyrazol-4- yl)quinolin-4-yl)ethan-1- amine and 4-(((tert- butoxycarbonyl)amino) methyl)-2-methylbenzoic acid following a procedure analogous to Example 4, Step 1-2) | ¹H NMR (400 MHz, MeOD) δ ppm 9.06 (s, 1H), 8.52 (br s, 1H), 8.35 (s, 1H), 8.29 (d, J = 8.6 Hz, 1H), 8.17 (s, 1H), 8.08 (d, J = 8.4 Hz, 1H), 7.87 (s, 1H), 7.78 (t, J = 7.6 Hz, 1H), 7.66 − 7.62 (m, 1H), 7.61 (s, 1H), 7.42 (d, J = 7.9 Hz, 1H), 7.36 − 7.30 (m, 2H), 6.05 (q, J = 6.9 Hz, 1H), 4.16 (s, 2H), 4.03 (br s, 2H), 4.02 (s, 3H), 2.38 (s, 3H), 1.76 (d, J = 7.0 Hz, 3H). LCMS (ESI): m/z = 497.3 [M + H]⁺. |
| | yl)quinolin-4-yl)ethyl)-4- | | |
| | (((thiazol-4-ylmethyl)amino) | | |
| | methyl)benzamide | | |

### Example 8: rel-(R)-5-(aminomethyl)-2-methyl-N-(1-(3-(1-methyl-1H-pyrazol-4-yl)naphthalen-1-yl)ethyl)benzamide, ENT-1

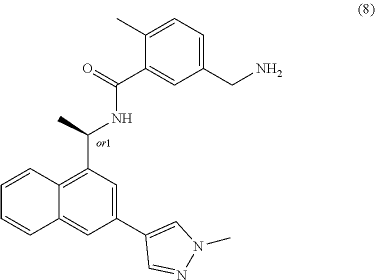 [SMILES: Cc1ccc(CN)cc1C(=O)N[C@H](C)c1cc(-c2cnn(C)c2)cc2ccccc12]

### Step 1. Preparation of tert-butyl(4-methyl-3-((1-(3-(1-methyl-1H-pyrazol-4-yl)naphthalen-1-yl)ethyl)carbamoyl)benzyl)carbamate

To a solution of 5-(((tert-butoxycarbonyl)amino)methyl)-2-methylbenzoic acid (60.0 mg, 0.226 mmol) in DMF (2.26 mL) was added DIEA (58.5 mg, 0.452 mmol) and HATU (129 mg, 0.339 mmol) at 25° C., the mixture was stirred at 25° C. for 3 h. Then 1-(3-(1-methyl-1H-pyrazol-4-yl)naphthalen-1-yl)ethan-1-amine (P5) (56.8 mg, 0.226 mmol) was added. The mixture was stirred at 25° C. for 24 h. The crude product was purified by prep-HPLC and dried by lyophilization to afford tert-butyl(4-methyl-3-((1-(3-(1-methyl-1H-pyrazol-4-yl)naphthalen-1-yl)ethyl)carbamoyl)benzyl)carbamate (68 mg, 60.3%) as a white solid. LCMS m/z=443.1 [M+H−tBu]⁺.

### Step 2. Preparation of 5-(aminomethyl)-2-methyl-N-(1-(3-(1-methyl-1H-pyrazol-4-yl)naphthalen-1-yl)ethyl)benzamide

To a stirred solution of tert-butyl(4-methyl-3-((1-(3-(1-methyl-1H-pyrazol-4-yl)naphthalen-1-yl)ethyl)carbamoyl)benzyl)carbamate (68.0 mg, 0.154 mmol) in dichloromethane (1.0 mL) was added 4M HCl/dioxane (1 mL) at 0° C. The mixture was stirred at 0° C. for 15 min and 25° C. for 2 h. LCMS showed the starting material was consumed and the desired product (m/z=399.0 [M+H]⁺) was observed. The reaction mixture was concentrated under vacuum to afford 5-(aminomethyl)-2-methyl-N-(1-(3-(1-methyl-1H-pyrazol-4-yl)naphthalen-1-yl)ethyl)benzamide (65 mg, 100% as HCl salt) as a yellow solid.

### Step 3. Chiral Separation

The racemic 5-(aminomethyl)-2-methyl-N-(1-(3-(1-methyl-1H-pyrazol-4-yl)naphthalen-1-yl)ethyl)benzamide (65.0 mg, 0.138 mmol) was separated via prep-SFC. After separation via prep-SFC, the two fractions were concentrated in vacuo and lyophilized to afford rel-(R)-5-(aminomethyl)-2-methyl-N-(1-(3-(1-methyl-1H-pyrazol-4-yl)naphthalen-1-yl)ethyl)benzamide, ENT-1 (first peak, 28.4 mg, 51.7%) and rel-(R)-5-(aminomethyl)-2-methyl-N-(1-(3-(1-methyl-1H-pyrazol-4-yl)naphthalen-1-yl)ethyl)benzamide, ENT-2 (second peak, 32.1 mg, 58.4%) as white solids.

### Prep-SFC Condition:

Column: DAICEL CHIRALPAK OD (250 mm*30 mm, 10 μm);
Mobile phase: 0.1% NH₃·H₂O IPA; B %: 50%-50%,
Flow Rate (mL/min): 80 mL/min.

rel-(R)-5-(aminomethyl)-2-methyl-N-(1-(3-(1-methyl-1H-pyrazol-4-yl)naphthalen-1-yl)ethyl)benzamide, ENT-1. ¹H NMR (400 MHz, MeOD-d₄) δ=8.24 (br d, J=9.2 Hz, 1H), 8.10 (s, 1H), 8.03-7.85 (m, 4H), 7.59-7.49 (m, 2H), 7.37-7.28 (m, 2H), 7.27-7.21 (m, 1H), 6.09 (q, J=6.8 Hz, 1H), 3.98 (s, 3H), 3.80 (s, 2H), 2.35 (s, 3H), 1.77 (d, J=6.8 Hz, 3H). LCMS 99.83% of purity, m/z=3993, [M+H]⁺. SFC 99.87% of chiral purity.

rel-(R)-5-(aminomethyl)-2-methyl-N-(1-(3-(1-methyl-1H-pyrazol-4-yl)naphthalen-1-yl)ethyl)benzamide, ENT-2. ¹H NMR (400 MHz, MeOD-d₄) δ=8.25 (br d, J=9.0 Hz, 1H), 8.13 (s, 1H), 8.04-7.85 (m, 4H), 7.60-7.48 (m, 2H), 7.46-7.33 (m, 3H), 6.12 (q, J=6.8 Hz, 1H), 4.09 (s, 2H), 3.99 (s, 2H), 2.39 (s, 3H), 1.78 (d, J=6.8 Hz, 3H). LCMS 100% of purity, m/z=399.3, [M+H]⁺. SFC 99.73% of chiral purity.

### Example 9: rel-(R)-5-(aminomethyl)-2-methyl-N-(1-(3-(1-methyl-1H-pyrazol-4-yl)naphthalen-1-yl)ethyl)benzamide, ENT-2

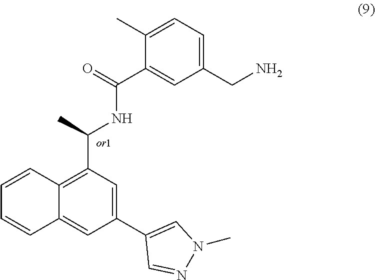 [SMILES: Cc1ccc(CN)cc1C(=O)N[C@H](C)c1cc(-c2cnn(C)c2)cc2ccccc12]

rel-(R)-5-(aminomethyl)-2-methyl-N-(1-(3-(1-methyl-1H-pyrazol-4-yl)naphthalen-1-yl)ethyl)benzamide, ENT-2 was obtained in Example 8, Step 3.

### Example 11: (R)-5-(aminomethyl)-2-methyl-N-(1-(2-(1-methyl-1H-pyrazol-4-yl)quinolin-4-yl)ethyl)benzamide

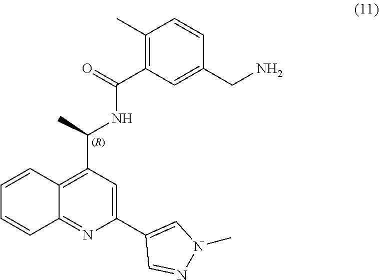

### Step 1. Preparation of (R-1-(2-(1-methyl-1H-pyrazol-4-yl)quinolin-4-yl)ethan-1-amine

To a solution of benzyl(R)-(1-(2-chloroquinolin-4-yl)ethyl)carbamate (P10) (2000.0 mg, 5.868 mmol) in 1,4-dioxane (50.0 mL) under N2 were added 1-methyl-4-(4,4,5,5-tetramethyl-1,3,2-dioxaborolan-2-yl)-1H-pyrazole (2440 mg, 11.7 mmol) and Pd(dppf)Cl₂ (429 mg, 0.587 mmol) and sat. aq. Na₂CO₃ (5.0 mL) at 20° C. The reaction mixture was stirred at 100° C. under N₂ for 15 h. LCMS showed a mass peak of the desired product. The reaction mixture was evaporated in vacuo and purified by silica gel chromatography via Biotage (40 g silicon column, PE:EA=1:0 to 0:1, EA:MeOH=1:0 to 0:1) to afford (R)-1-(2-(1-methyl-1H-pyrazol-4-yl)quinolin-4-yl)ethan-1-amine (606 mg, 26.7%) as the yellow solid (LCMS (ESI): m/z=253 [M+H]⁺) and benzyl(R)-(1-(2-(1-methyl-1H-pyrazol-4-yl)quinolin-4-yl)ethyl)carbamate (1023 mg, 69.1%) as a red solid.

### Step 2. Preparation of tert-butyl(R)-(4-methyl-3-((1-(2-(1-methyl-1H-pyrazol-4-yl)quinolin-4-yl)ethyl)carbamoyl)benzyl)carbamate

To a solution of 5-(((tert-butoxycarbonyl)amino)methyl)-2-methylbenzoic acid (126 mg, 0.476 mmol) in DMF (10 mL) were added HATU (271 mg, 0.713 mmol) and TEA (144 mg, 1.43 mmol) and (R)-1-(2-(1-methyl-1H-pyrazol-4-yl)quinolin-4-yl)ethan-1-amine (120.0 mg, 0.476 mmol) at 20° C. The reaction mixture was stirred at 20° C. for 15 h. LCMS showed a mass peak of the desired product. The reaction mixture was partitioned between EA and H₂O (50/50 mL). The organic layer was separated, and the aqueous layer was re-extracted with EtOAc (50 mL). The combined organic layer was washed with brine, dried with anhydrous Na₂SO₄, and evaporated in vacuo to afford the crude tert-butyl(R)-(4-methyl-3-((1-(2-(1-methyl-1H-pyrazol-4-yl)quinolin-4-yl)ethyl)carbamoyl)benzyl)carbamate (238 mg) as a yellow solid. LCMS (ESI): m/z=500 [M+H]⁺.

### Step 3. Preparation of (R)-5-(aminomethyl)-2-methyl-N-(1-(2-(1-methyl-1H-pyrazol-4-yl)quinolin-4-yl)ethyl)benzamide

To a solution of tert-butyl(R)-(4-methyl-3-((1-(2-(1-methyl-1H-pyrazol-4-yl)quinolin-4-yl)ethyl)carbamoyl)benzyl)carbamate (210 mg, 0.420 mmol) in dichloromethane (15 mL) was added HCl (3 mL, 4 M) at 20° C. The reaction mixture was stirred at 20° C. for 30 min. LCMS showed a mass peak of the desired product. The reaction mixture was evaporated in vacuo to afford a crude, which was redissolved in MeOH (8 mL), basified with ammonium hydroxide (3 mL) at 0° C., and evaporated again in vacuo to afford a residue. The residue was dissolved in 5 mL DMF and purified by prep-HPLC to afford (R)-5-(aminomethyl)-2-methyl-N-(1-(2-(1-methyl-1H-pyrazol-4-yl)quinolin-4-yl)ethyl)benzamide (69 mg, 41% yield) as a white solid. ¹H NMR (400 MHz, MeOD) δ ppm 8.35 (s, 1H), 8.30 (d, J=7.9 Hz, 1H), 8.19 (s, 1H), 8.08 (d, J=8.4 Hz, 1H), 7.88 (s, 1H), 7.79 (t, J=7.7 Hz, 1H), 7.63 (t, J=7.1 Hz, 1H), 7.38-7.32 (m, 2H), 7.25 (d, J=7.9 Hz, 1H), 6.06 (q, J=7.0 Hz, 1H), 4.02 (s, 3H), 3.82 (s, 2H), 2.35 (s, 3H), 1.76 (d, J=7.0 Hz, 3H). LCMS (ESI): m/z=400 [M+H]⁺.

### Prep-HPLC Condition:

Column: Boston Prime C18 150*30 mm*5 μm;
Mobile phase: water (ammonium hydroxide v/v)-acetonitrile; B %: 12%-52%,
Flow Rate (mL/min): 30 mL/min.

### Example 12: (R)-2-methyl-N-(1-(2-(1-methyl-1H-pyrazol-3-yl)quinolin-4-yl)ethyl)benzamide

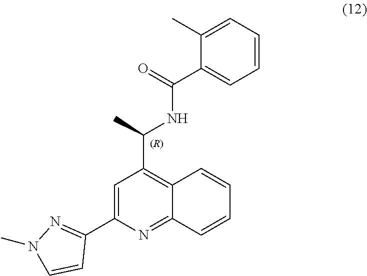

Using a procedure analogous to Example 4 Step 1 with (R)-1-(2-(1-methyl-1H-pyrazol-3-yl)quinolin-4-yl)ethan-1-amine (180.0 mg, 0.713 mmol, prepared in a manner analogous to (R)-1-(2-(1-methyl-1H-pyrazol-4-yl)quinolin-4-yl)ethan-1-amine) and 2-methylbenzoic acid (97.1 mg, 0.713 mmol) as the reactants, affords (R)-2-methyl-N-(1-(2-(1-methyl-1H-pyrazol-3-yl)quinolin-4-yl)ethyl)benzamide (69.69 mg, 26.4%) as a white solid. ¹H NMR (400 MHz, MeOD-d₄) δ=8.32-8.27 (m, 2H), 8.14 (d, J=8.4 Hz, 1H), 7.78 (t, J=7.5 Hz, 1H), 7.71 (d, J=2.2 Hz, 1H), 7.67-7.62 (m, 1H), 7.40 (d, J=7.0 Hz, 1H), 7.36-7.30 (m, 1H), 7.27-7.21 (m, 2H), 7.08 (d, J=2.2 Hz, 1H), 6.03 (d, J=7.0 Hz, 1H), 4.01 (s, 3H), 236 (s, 3H), 1.74 (d, J=7.0 Hz, 3H). LCMS (ESI): m/z=371.3 [M+H]⁺.

### Example 15: (R)—N-methyl-N-(4-methyl-3-(((R)-1-(2-(1-methyl-1H-pyrazol-4-yl)quinolin-4-yl)ethyl)carbamoyl)phenyl)piperidine-2-carboxamide

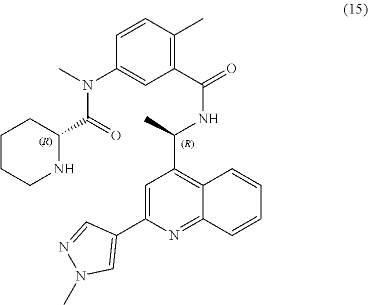

### Step 1. Preparation of tert-butyl(R)-2 (3-(methoxycarbonyl)-4-methylphenyl)(methyl) carbamoyl)piperidine-1-carboxylate

To a solution of tert-butyl(R)-2-((3-(methoxycarbonyl)-4-methylphenyl)carbamoyl)piperidine-1-carboxylate (340.0 mg, 0.903 mmol) in THF (20 mL) under N₂ was added NaH (54.2 mg, 1.35 mmol) at 0° C. The reaction mixture was stirred at 0° C. under N for 30 min before adding CH₃I (192 mg, 1.35 mmol). The reaction mixture was then stirred at 20° C. under N₂ for 15 h. LCMS showed a mass peak of the desired product. The reaction was quenched with 3 mL sat. aq. NH₄Cl at 0° C., then partitioned between EA and H₂O (50/50 mL). The organic layer was separated and the aqueous layer was re-extracted with EtOAc (50 mL). The combined organic layer was washed with brine, dried over anhydrous Na₂SO₄, and evaporated in vacuo to afford tert-butyl(R)-2-((3-(methoxycarbonyl)-4-methylphenyl)(methyl)carbamoyl)piperidine-1-carboxylate (353 mg) as a yellow oil. LCMS (ESI): m/z=391 [M+H]⁺.

### Step 2. Preparation of (R)-5-(1-(tert-butoxycarbonyl)-N-methylpiperidine-2-carboxamido)-2-methylbenzoic acid

To a solution of tert-butyl(R)-2-((3-(methoxycarbonyl)-4-methylphenyl)(methyl)carbamoyl) piperidine-1-carboxylate (350 mg, 0.896 mmol) in MeOH (20 mL) was added 10% aq. NaOH (3 mL) at 20° C. The reaction mixture was stirred at 20° C. for 5 h. LCMS showed a mass peak of the desired product. The reaction mixture was then acidified to pH=6 with 1N HCl at 0° C. and partitioned between EA and H₂O (100/50 mL). The organic layer was separated and the aqueous layer was re-extracted with EtOAc (50 mL). The combined organic layer was washed with brine, dried over anhydrous Na₂SO₄, and evaporated in vacuo to afford (R)-5-(1-(tert-butoxycarbonyl)-N-methylpiperidine-2-carboxamido)-2-methylbenzoic acid (337 mg) as a white solid. LCMS (ESI): m/z=377 [M+H]⁺.

### Step 3. Preparation of tert-butyl(R)-2-(methyl(4-methyl-3-(((R)-1-(2-(1-methyl-1H-pyrazol-4-yl)quinolin-4-yl)ethyl)carbamoyl)phenyl)carbamoyl)piperidine-1-carboxylate

Using a procedure analogous to Example 4 Step 1 with (R)-5-(1-(tert-butoxycarbonyl)-N-methylpiperidine-2-carboxamido)-2-methylbenzoic acid (150.0 mg, 0.398 mmol) and (R)-1-(2-(1-methyl-1H-pyrazol-4-yl)quinolin-4-yl)ethan-1-amine (101 mg, 0.398 mmol) as the reactants affords tert-butyl(R)-2-(methyl(4-methyl-3-(((R)-1-(2-(1-methyl-1H-pyrazol-4-yl)quinolin-4-yl)ethyl)carbamoyl)phenyl)carbamoyl)piperidine-1-carboxylate (243 mg, 99.9%) as a yellow solid. LCMS (ESI): m/z=611 [M+H]⁺.

### Step 4. Preparation of (R)—N-methyl-N-(4-methyl-3-(((R)-1-(2-(1-methyl-1H-pyrazol-4-yl)quinolin-4-yl)ethyl)carbamoyl)phenyl)piperidine-2-carboxamide

Using a procedure analogous to Example 4 Step 2 with tert-butyl(R)-2-(methyl(4-methyl-3-(((R)-1-(2-(1-methyl-1H-pyrazol-4-yl)quinolin-4-yl)ethyl)carbamoyl)phenyl)carbamoyl)piperidine-1-carboxylate (240 mg, 0.393 mmol) as the reactant affords (R)—N-methyl-N-(4-methyl-3-(((R)-1-(2-(1-methyl-1H-pyrazol-4-yl)quinolin-4-yl)ethyl)carbamoyl)phenyl)piperidine-2-carboxamide (23 mg, 11%) as a white solid. ¹H NMR (400 MHz, MeOD) 5 ppm 8.53 (br s, 1H), 8.42-8.25 (m, 2H), 8.17 (s, 1H), 8.08 (d, J=8.3 Hz, 1H), 7.87 (s, 1H), 7.79 (t, J=7.5 Hz, 1H), 7.64 (t, J=7.7 Hz, 1H), 7.46 (br d, J=8.1 Hz, 1H), 7.41-7.34 (m, 1H), 7.32 (s, 1H), 6.08 (br d, J=7.5 Hz, 1H), 4.02 (s, 3H), 3.73 (br d, J=12.2 Hz, 1H), 3.31-3.28 (m, 3H), 2.86 (br t, J=12.1 Hz, 1H), 2.44 (s, 3H), 1.86 (br d, J=13.2 Hz, 1H), 1.79 (br d, J=6.8 Hz, 3H), 1.76-1.48 (m, 5H). LCMS (ESI): m/z=511 [M+H]⁺.

### Example 16: (R)—N-(1-(2-(4H-1,2,4-triazol-4-yl)quinolin-4-yl)ethyl)-5-(aminomethyl)-2-methylbenzamide

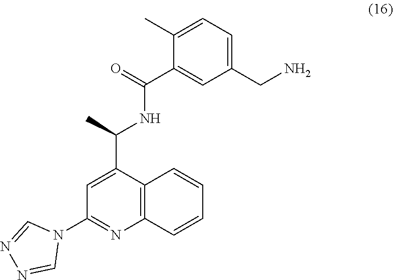 [SMILES: Cc1ccc(CN)cc1C(=O)N[C@H](C)c1cc(-n2cnnc2)nc2ccccc12]

### Step 1. Preparation of benzyl(R)-(1-(2-((diphenylmethylene)amino)quinolin-4-yl)ethyl) carbamate

To solution of benzyl(R)-(1-(2-chloroquinolin-4-yl)ethyl)carbamate (800.0 mg, 2.35 mmol) in toluene (15.0 mL) were added diphenyl methanimine (851 mg, 4.69 mmol), t-BuONa (271 mg, 2.82 mmol), Pd₂(dba)₃ (215 mg, 0.235 mmol) and BINAP (146 mg, 0.235 mmol) at 20° C. The reaction mixture was stirred at 110° C. under N₂ for 15 h. LCMS showed a mass peak of the desired product. The reaction mixture was then concentrated in vacuo to give a crude, which was purified by flash chromatography (40 g silica gel column, ethyl acetate in PE from 0 to 100%) to afford benzyl(R)-(1-(2-((diphenylmethylene)amino)quinolin-4-yl)ethyl)carbamate (600 mg, 52.6%) as a yellow solid. LCMS (ESI): m/z=486.3 [M+H]⁺.

### Step 2. Preparation of benzyl(R)-(1-(2-aminoquinolin-4-yl)ethyl)carbamate

To a solution of benzyl(R)-(1-(2-((diphenylmethylene)amino)quinolin-4-yl)ethyl)carbamate (600.0 mg, 1.24 nmmol) in THF (20.0 mL) was added 1M HCl (20 mL) at 20° C. The reaction was then stirred at 20° C. for 15 h. LCMS showed a mass peak was the desired product. The reaction mixture was then evaporated in vacuo then purified by silica gel chromatography using a Biotage (40 g column, EtOAc in PE from 0 to 100%) to afford benzyl(R)-(1-(2-aminoquinolin-4-yl)ethyl)carbamate (300 mg, 75.5%) as a yellow oil. LCMS (ESI): m/z=322.1 [M+H]⁺.

### Step 3. Preparation of benzyl(R)-(1-(2-(4H-1,2,4-triazol-4-yl)quinolin-4-yl)ethyl)carbamate

To a solution of benzyl(R)-(1-(2-aminoquinolin-4-yl)ethyl)carbamate (300.0 mg, 0.933 mmol) in toluene (10.0 mL) were added (E)-N′—((E)-(dimethylamino)methylene)-N,N-dimethylformohydrazonamide (265.0 mg, 1.87 mmol) and p-toluenesulfonic acid (32.1 mg, 0.187 mmol) at 30° C., The reaction mixture was then stirred at 110° C. for 18 h. LCMS showed a mass peak of the desired product. The reaction mixture was then evaporated in vacuo and purified by silica gel chromatography using a Biotage (20 g column, EtOAc in PE from 0 to 100%) to afford benzyl(R)-(1-(2-(4H-1,2,4-triazol-4-yl)quinolin-4-yl)ethyl)carbamate (230 mg, 66%) as a yellow oil. LCMS (ESI): m/z=374.1, [M+H]⁺.

### Step 4. Preparation of (R)-1-(2-(4H-1,2,4-triazol-4-yl)quinolin-4-yl)ethan-1-amine

To a solution of benzyl(R)-(1-(2-(4H-1,2,4-triazol-4-yl)quinolin-4-yl)ethyl)carbamate (230.0 mg, 0.616 mmol) in i-PrOH (20.0 mL) was added wet Pd/C (40 mg, 0.38 mmol) at 30° C. The mixture was degassed and refilled with H₂ three times, then stirred at 30° C. under 15 psi (1.03421 bar) for 16 h. LCMS showed a mass peak of the desired product. The reaction mixture was then filtered, and the filtrate was evaporated in vacuo to afford (R)-1-(2-(4H-1,2,4-triazol-4-yl)quinolin-4-yl)ethan-1-amine (100 mg, 67.9%) as a yellow oil. LCMS (ESI): m/z=240.1 [M+H]⁺.

### Step 5. Preparation of tert-butyl(R)-(3-((1(2-(4H-1,2,4-triazol-4-yl)quinolin-4-yl)ethyl)carbamoyl)-4-methylbenzyl)carbamate

Using a procedure analogous to Example 4, Step 1 with (R)-1-(2-(4H-1,2,4-triazol-4-yl)quinolin-4-yl)ethan-1-amine (100.0 mg, 0.418 mmol) and 5-(((tert-butoxycarbonyl)amino)methyl)-2-methylbenzoic acid (60.0 mg, 0.23 mmol) as the reactants affords tert-butyl(R)-(3-((1-(2-(4H-1,2,4-triazol-4-yl)quinolin-4-yl)ethyl)carbamoyl)-4-methylbenzyl)carbamate (68 mg, 33%) as a purple solid. LCMS (ESI): m/z=487.2 [M+H]⁺.

### Step 6. Preparation of (R)—N-(1-(2-(4H-1,2,4-triazol-4-yl)quinolin-4-yl)ethyl)-5-(aminomethyl)-2-methylbenzamide

Using a procedure analogous to Example 4 Step 2 with tert-butyl(R)-(3-((1-(2-(4H-1,2,4-triazol-4-yl)quinolin-4-yl)ethyl)carbamoyl)-4-methylbenzyl)carbamate (68 mg, 0.14 mmol) as the reactant affords (R)—N-(1-(2-(4H-1,2,4-triazol-4-yl)quinolin-4-yl)ethyl)-5-(aminomethyl)-2-methylbenzamide (20.68 mg, 38.0%) as a white solid. ¹H NMR (400 MHz, MeOD-d₄) δ=9.44 (s, 2H), 8.52 (s, 1H), 8.42 (d, J=8.3 Hz, 1H), 8.14 (d, J=8.3 Hz, 1H), 7.95 (s, 1H), 7.89 (t, J=7.3 Hz, 1H), 7.78-7.72 (m, 1H), 7.44-7.39 (m, 2H), 7.35-7.29 (m, 1H), 6.12 (d, J=7.3 Hz, 1H), 4.03 (s, 2H), 2.36 (s, 3H), 1.80 (d, J=7.3 Hz, 3H). LCMS (ESI): m/z=387.3 [M+H]⁺.

### Example 17: (R)—N-(1-(2-(l H-pyrazol-4-yl)quinolin-4-yl)ethyl)-2-methyl-5-((methylamino)methyl)benzamide

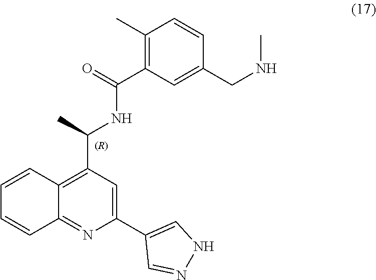

### Step 1. Preparation of benzyl((1R)-1-(2-(1(tetrahydro-2H-pyran-2-yl)-1H-pyrazol-4-yl)quinolin-4-yl)ethyl)carbamate

To a solution of benzyl(R)-(1-(2-chloroquinolin-4-yl)ethyl)carbamate (700.0 mg, 2.05 mmol) in 1,4-Dioxane (15.0 mL) were added 1-(tetrahydro-2H-pyran-2-yl)-4-(4,4,5,5-tetramethyl-1,3,2-dioxaborolan-2-yl)-1H-pyrazole (857 mg, 3.08 mmol) and Pd(dppf)Cl₂ (150 mg, 0.205 mmol) and sat. aq. Na₂CO₃ (1.0 mL) at 20° C. The reaction mixture was stirred at 100° C. for 2 h. LCMS showed a mass peak of the desired product. The reaction mixture was then evaporated in vacuo and purified by silica gel chromatography using a Biotage (40 g column, PE:EA=1:0 to 0:1) to afford benzyl((1R)-1-(2-(1-(tetrahydro-2H-pyran-2-yl)-1H-pyrazol-4-yl)quinolin-4-yl)ethyl)carbamate (850 mg, 90.6%) as a yellow oil. LCMS (ESI): m/z=457 [M+H]⁺.

### Step 2. Preparation of (1R)-1-(2-(1-(tetrahydro-2H-pyran-2-yl)-1H-pyrazol-4-yl)quinolin-4-yl)ethan-1-amine

To a solution of benzyl((1R)-1-(2-(1-(tetrahydro-2H-pyran-2-yl)-1H-pyrazol-4-yl)quinolin-4-yl)ethyl)carbamate (850.0 mg, 1.86 mmol) in iPrOH (40.0 mL) was added wet Pd/C (40 mg, 0.38 mmol) at 30° C. The reaction mixture was degassed and refilled with H₂ three times, then stirred at 30° C. under 15 psi (1.03421 bar) for 16 h. LCMS showed a mass peak of the desired product. The reaction mixture was then filtered, and the filtrate was evaporated in vacuo to afford (1R)-1-(2-(1-(tetrahydro-2H-pyran-2-yl)-1H-pyrazol-4-yl)quinolin-4-yl)ethan-1-amine (470 mg, 78.3%) as a yellow oil. LCMS (ESI): m/z=323 [M+H]⁺.

### Step 3. Preparation of tert-butyl methyl(4-methyl-3-(((1R)-1-(21-(tetrahydro-2H-pyran-2-yl)-1H-pyrazol-4-yl)quinolin-4-yl)ethyl)carbamoyl)benzyl)carbamate

Using a procedure analogous to Example 4, Step 1 with (1R)-1-(2-(1-(tetrahydro-2H-pyran-2-yl)-1H-pyrazol-4-yl)quinolin-4-yl)ethan-1-amine (250.0 mg, 0.775 mmol) and 5-(((tert-butoxycarbonyl)(methyl)amino)methyl)-2-methylbenzoic acid (217 mg, 0.775 mmol) as the reactants affords tert-butyl methyl(4-methyl-3-(((1R)-1-(2-(1-(tetrahydro-2H-pyran-2-yl)-1H-pyrazol-4-yl)quinolin-4-yl)ethyl)carbamoyl)benzyl)carbamate (400 mg, 88.4%) as a yellow oil. LCMS (ESI): m/z=584 [M+H]⁺.

### Step 4. Preparation of (R)—N-(1-(2-(1H-pyrazol-4-yl)quinolin-4-yl)ethyl)-2-methyl-5-((methylamino)methyl)benzamide

Using a procedure analogous to Example 4, Step 2 with tert-butyl methyl(4-methyl-3-(((1R)-1-(2-(1-(tetrahydro-2H-pyran-2-yl)-1H-pyrazol-4-yl)quinolin-4-yl)ethyl)carbamoyl)benzyl) carbamate (400.0 mg, 0.685 mmol) as the reactant affords (R)—N-(1-(2-(1H-pyrazol-4-yl)quinolin-4-yl)ethyl)-2-methyl-5-((methylamino)methyl)benzamide (58 mg, 21.2%) as a white solid. ¹H NMR (400 MHz, MeOD) δ ppm 8.37 (s, 2H), 8.30 (d, J=8.4 Hz, 1H), 8.10 (d, J=7.9 Hz, 1H), 7.94 (s, 1H), 7.79 (t, J=7.6 Hz, 1H), 7.64 (t, J=7.6 Hz, 1H), 7.36 (s, 1H), 7.33 (d, J=7.5 Hz, 1H), 7.25 (d, J=7.5 Hz, 1H), 6.07 (q, J=7.2 Hz, 1H), 5.10-4.96 (m, 3H), 3.72 (s, 2H), 2.40 (s, 3H), 2.36 (s, 3H), 1.76 (d, J=7.0 Hz, 3H). LCMS (ESI): m/z=400, [M+H]⁺.

The following example was prepared in a similar manner to Example 17 using an appropriate amine and an appropriate carboxylic acid.

| Ex. | Structure; | Analogous method, | |
|---|---|---|---|
| # | Compound Name | reactants | Characterizing Data |
| 18 | 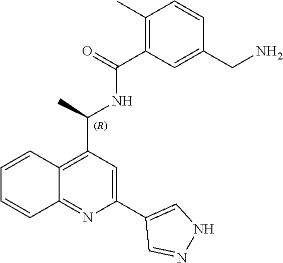 | Example 4, Step 1, (1R)-1-(2- (1-(tetrahydro-2H-pyran-2-yl)- 1H-pyrazol-4-yl)quinolin-4- yl)ethan-1-amine and 5- (((tert-butoxycarbonyl)amino) methyl)-2-methylbenzoic acid (P8); Then Example 4, Step 2, tert- butyl (4-methyl-3-(((1R)-1-(2- (1-(tetrahydro-2H-pyran-2-yl)- 1H-pyrazol-4-yl)quinolin-4- yl)ethyl) carbamoyl)benzyl) carbamate | ¹H NMR (400 MHz, MeOD) δ ppm 8.36 (s, 2H), 8.31 (d, J = 8.3 Hz, 1H), 8.10 (d, J = 8.3 Hz, 1H), 7.94 (s, 1H), 7.79 (t, J = 7.7 Hz, 1H), 7.64 (t, J = 7.7 Hz, 1H), 7.37 (s, 1H), 7.35 (d, J = 7.5 Hz, 1H), 7.27-7.23 (m, 1H), 6.07 (q, J = 7.0 Hz, 1H), 3.83 (s, 2H), 2.36 (s, 3H), 1.77 (d, J = 7.0 Hz, 3H). LCMS (ESI): m/z = 386 [M + H]⁺. |
| | (R)-N-(1-(2-(1H-pyrazol-4- | | |
| | yl)quinolin-4-yl)ethyl)-5- | | |
| | (aminomethyl)-2- | | |
| | methylbenzamide | | |

### Example 19: methyl(R)-5-(4-(1-(5-(aminomethyl)-2-methylbenzamido)ethyl)quinolin-2-yl)-1H-pyrrole-3-carboxylate

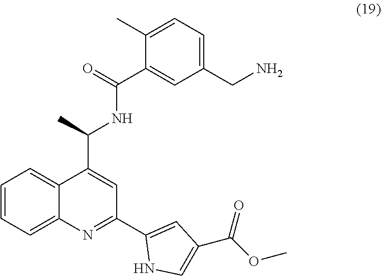 [SMILES: COC(=O)c1c[nH]c(-c2cc([C@@H](C)NC(=O)c3cc(CN)ccc3C)c3ccccc3n2)c1]

### Step 1. Preparation of methyl(R)-5-(4-(1-aminoethyl)quinolin-2-yl)-1H-pyrrole-3-carboxylate

Using a procedure analogous to Example 11 Step 1 with benzyl(R)-(1-(2-chloroquinolin-4-yl)ethyl)carbamate (1150 mg, 3.39 mmol) and methyl 5-(4,4,5,5-tetramethyl-1,3,2-dioxaborolan-2-yl)-1H-pyrrole-3-carboxylate (850.0 mg, 3.39 mmol) as the reactants affords methyl(R)-5-(4-(1-aminoethyl)quinolin-2-yl)-1H-pyrrole-3-carboxylate (505 mg, 50.5%) as the yellow solid. LCMS (ESI): m/z=296 [M+H]⁺.

### Step 2. Preparation of methyl(R)-5-(4-(1-(5-(((tert-butoxycarbonyl)amino)methyl)-2-methylbenzamido)ethyl)quinolin-2-yl)-1H-pyrrole-3-carboxylate

Using a procedure analogous to Example 11 Step 2 with 5-(((tert-butoxycarbonyl)amino)methyl)-2-methylbenzoic acid (359 mg, 1.35 mmol) and methyl(R)-5-(4-(1-aminoethyl)quinolin-2-yl)-1H-pyrrole-3-carboxylate (400.0 mg, 1.35 mmol) as the reactants affords methyl(R)-5-(4-(1-(5-(((tert-butoxycarbonyl)amino)methyl)-2-methylbenzamido)ethyl)quinolin-2-yl)-1H-pyrrole-3-carboxylate (735 mg, 100%) as a yellow solid. LCMS (ESI): m/z=543 [M+H]⁺.

### Step 3. Preparation of methyl(R)-5-(4-(1-(5-(aminomethyl)-2-methylbenzamido)ethyl)quinolin-2-yl)-1H-pyrrole-3-carboxylate

Using a procedure analogous to Example 11 Step 3 with methyl(R)-5-(4-(1-(5-(((tert-butoxycarbonyl)amino)methyl)-2-methylbenzamido)ethyl)quinolin-2-yl)-1H-pyrrole-3-carboxylate (100 mg, 0.184 mmol) as the reactant affords methyl(R)-5-(4-(1-(5-(aminomethyl)-2-methylbenzamido)ethyl)quinolin-2-yl)-1H-pyrrole-3-carboxylate (7 mg, 8%) as a yellow solid. ¹H NMR (400 MHz, MeOD) 5 ppm 8.55 (s, 1H), 8.27 (d, J=7.9 Hz, 1H), 8.10 (d, J=7.7 Hz, 1H), 7.94 (s, 1H), 777 (t, J=7.2 Hz, 1H), 7.67-7.59 (m, 2H), 7.49-7.40 (m, 2H), 736 (d, J=7.4 Hz, 1H), 7.32 (s, 1H), 6.09-6.02 (m, 1H), 4.06 (s, 2H), 3.86 (s, 3H), 2.40 (s, 3H), 1.75 (d, J=7.0 Hz, 3H). LCMS (ESI): m/z=443 [M+H]⁺.

### Example 20: (R)-5-(4-(1-(5-(aminomethyl)-2-methylbenzamido)ethyl)quinolin-2-yl)-1H-pyrrole-3-carboxylic acid

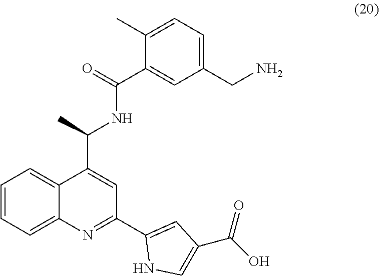 [SMILES: Cc1ccc(CN)cc1C(=O)N[C@H](C)c1cc(-c2cc(C(=O)O)c[nH]2)nc2ccccc12]

### Step 1. Preparation of (R-5-(4-(1-(5-((tert-butoxycarbonyl)amino)methyl)-2-methylbenzamido)ethyl)quinolin-2-yl)-1H-pyrrole-3-carboxylic acid

To a solution of methyl(R)-5-(4-(1-(5-(((tert-butoxycarbonyl)amino)methyl)-2-methylbenzamido)ethyl)quinolin-2-yl)-1H-pyrrole-3-carboxylate (550 mg, 1.01 mmol, prepared in Example 19, Step 2) in MeOH (20 mL) was added 10% NaOH (5 mL) at 20° C. The reaction mixture was stirred at 50° C. for 15 h. LCMS showed a mass peak of the desired product. The reaction mixture was then acidified to pH=1 with 1N HCl at 0° C. and partitioned between EA and H₂O (50/50 mL). The organic layer was separated, and the aqueous layer was re-extracted with EtOAc (50 mL). The combined organic layer was washed with brine, dried with anhydrous Na₂SO₄, and evaporated in vacuo to afford (R)-5-(4-(1-(5-(((tert-butoxycarbonyl)amino)methyl)-2-methylbenzamido)ethyl)quinolin-2-yl)-1H-pyrrole-3-carboxylic acid (536 mg) as a yellow solid. LCMS (ESI): m/z=529 [M+H].

### Step 2. Preparation of (R)-5-(4-(1-(5-(aminomethyl)-2-methylbenzamido)ethyl)quinolin-2-yl)-1H-pyrrole-3-carboxylic acid

Using a procedure analogous to Example 4 Step 2 with (R)-5-(4-(1-(5-(((tert-butoxycarbonyl)amino)methyl)-2-methylbenzamido)ethyl)quinolin-2-yl)-1H-pyrrole-3-carboxylic acid (150 mg, 0.284 mmol) as the reactant affords (R)-5-(4-(1-(5-(aminomethyl)-2-methylbenzamido)ethyl)quinolin-2-yl)-1H-pyrrole-3-carboxylic acid (22 mg, 18%) as a yellow solid. ¹H NMR (400 MHz, MeOD) δ ppm 9.02 (d, J=8.1 Hz, 1H), 8.24 (d, J=8.1 Hz, 1H), 8.05 (s, 1H), 8.02 (d, J=8.4 Hz, 1H), 7.76 (t, J=7.2 Hz, 1H), 7.59 (t, J=7.1 Hz, 1H), 7.43 (d, J=4.1 Hz, 2H), 7.32 (d, J=7.4 Hz, 1H), 7.27-7.25 (m, 1H), 7.20 (d, J=7.9 Hz, 1H), 5.92 (quin, J=7.1 Hz, 1H), 3.80-3.55 (m, 2H), 2.26 (s, 3H), 1.60 (d, J=6.9 Hz, 3H). LCMS (ESI): m/z=429 [M+H]⁺.

### Example 21: (R)-5-(4-(1-(5-(aminomethyl)-2-methylbenzamido)ethyl)quinolin-2-yl)-1H-pyrrole-3-carboxamide

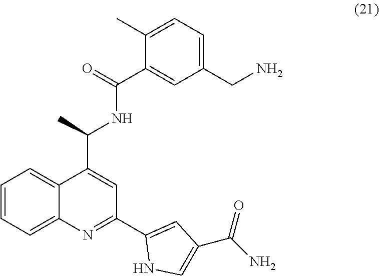 [SMILES: Cc1ccc(CN)cc1C(=O)N[C@H](C)c1cc(-c2cc(C(N)=O)c[nH]2)nc2ccccc12]

### Step 1. Preparation of tert-butyl(R) 3-((1-(2-(4-carbamoyl-1H-pyrrol-2-yl)quinolin-4-yl)ethyl)carbamoyl)-4-methylbenzyl)carbamate

To a solution of (R)-5-(4-(1-(5-(((tert-butoxycarbonyl)amino)methyl)-2-methylbenzamido)ethyl)quinolin-2-yl)-1H-pyrrole-3-carboxylic acid (170 mg, 0.322 mmol) in DMF (5 mL) were added HATU (183 mg, 0.482 mmol), TEA (163 mg, 1.61 mmol) and NH₄Cl (51.6 mg, 0.965 mmol) at 20° C. The reaction mixture was stirred at 20° C. for 15 h. LCMS showed a mass peak of the desired product. The reaction mixture was then partitioned between EA and H₂O (100/50 mL). The organic layer was separated, and the aqueous layer was re-extracted with EtOAc (50 mL). The combined organic layer was washed with brine, dried with anhydrous Na₂SO₄, and evaporated in vacuo to afford tert-butyl(R)-(3-((1-(2-(4-carbamoyl-1H-pyrrol-2-yl)quinolin-4-yl)ethyl)carbamoyl)-4-methylbenzyl)carbamate (170 mg, 100%) as the yellow solid. LCMS (ESI): m/z=529.2 [M+H]⁺.

### Step 2. Preparation of (R)-5-(4-(1-(5-(aminomethyl)-2-methylbenzamido)ethyl)quinolin-2-yl)-1H-pyrrole-3-carboxamide

Using a procedure analogous to Example 4, Step 2 with tert-butyl(R)-(3-((1-(2-(4-carbamoyl-1H-pyrrol-2-yl)quinolin-4-yl)ethyl)carbamoyl)-4-methylbenzyl)carbamate (150 mg, 0.284 mmol) as the reactant affords (R)-5-(4-(1-(5-(aminomethyl)-2-methylbenzamido)ethyl)quinolin-2-yl)-1H-pyrrole-3-carboxamide (22 mg, 16%) as a yellow solid. ¹H NMR (400 MHz, MeOD) 5 ppm 8.53 (s, 1H), 8.26 (d, J=8.4 Hz, 1H), 8.10 (d, J=7.9 Hz, 1H), 7.94 (s, 1H), 7.77 (t, J=7.7 Hz, 1H), 7.65 (s, 1H), 7.64-7.59 (m, 1H), 7.47 (s, 1H), 7.46 (d, J=7.6 Hz, 1H), 7.39-7.36 (m, 1H), 734 (s, 1H), 6.09-6.03 (m, 1H), 4.13 (s, 2H), 2.42 (s, 3H), 1.76 (d, J=6.8 Hz, 3H). LCMS (ESI): m/z=428 [M+H]⁺.

### Example 22: (R)-5-(4-(1-(5-(aminomethyl)-2-methylbenzamido)ethyl)quinolin-2-yl)-N-methyl-1H-pyrrole-3-carboxamide

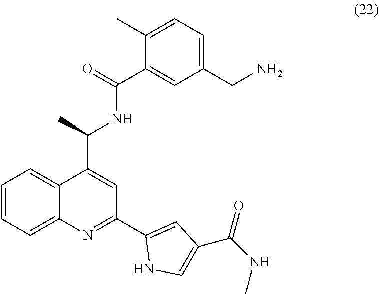 [SMILES: CNC(=O)c1c[nH]c(-c2cc([C@@H](C)NC(=O)c3cc(CN)ccc3C)c3ccccc3n2)c1]

### Step 1. Preparation of tert-butyl(R)-(4-methyl-3 (1-(2-(4-(methylcarbamoyl)-1H-pyrrol-2-yl)quinolin-4-yl)ethyl)carbamoyl)benzyl)carbamate

To a solution of (R)-5-(4-(1-(5-(((tert-butoxycarbonyl)amino)methyl)-2-methylbenzamido)ethyl) quinolin-2-yl)-1H-pyrrole-3-carboxylic acid (170 mg, 0.322 mmol, prepared in Example 20, Step 1) in DMF (5 mL) were added HATU (183 mg, 0.482 mmol), TEA (163 mg, 1.61 mmol) and CH₃NH₂·HCl (65.1 mg, 0.965 mmol) at 20° C. The reaction mixture was stirred at 20° C. for 15 h. LCMS showed a mass peak of the desired product. The reaction mixture was then partitioned between EA and H₂O (100/50 mL). The organic layer was separated, and the aqueous layer was re-extracted with EtOAc (50 mL). The combined organic layer was washed with brine, dried with anhydrous Na₂SO₄, and evaporated in vacuo to afford tert-butyl(R)-(4-methyl-3-((1-(2-(4-(methylcarbamoyl)-1H-pyrrol-2-yl)quinolin-4-yl)ethyl)carbamoyl)benzyl)carbamate (174 mg, 99.9%) as a yellow solid. LCMS (ESI): m/z=542, [M+H]⁺.

### Step 2. Preparation of (R)-5-(4-(1-(5-(aminomethyl)-2-methylbenzamido)ethyl)quinolin-2-yl)-N-methyl-1H-pyrrole-3-carboxamide

Using a procedure analogous to Example 4 Step 2 with tert-butyl(R)-(4-methyl-3-((1-(2-(4-(methylcarbamoyl)-1H-pyrrol-2-yl)quinolin-4-yl)ethyl)carbamoyl)benzyl)carbamate (150 mg, 0.277 mmol) as the reactant affords (R)-5-(4-(1-(5-(aminomethyl)-2-methylbenzamido)ethyl) quinolin-2-yl)-N-methyl-1H-pyrrole-3-carboxamide (24 mg, 18%) as a yellow solid. H NMR (400 MHz, MeOD) δ ppm 8.54 (s, 1H), 8.26 (d, J=8.1 Hz, 1H), 8.09 (d, J=8.4 Hz, 1H), 7.93 (s, 1H), 7.77 (t, J=7.7 Hz, 1H), 7.61 (t, J=7.7 Hz, 1H), 7.57 (s, 1H), 7.49-7.44 (m, 2H), 7.41-7.34 (m, 1H), 7.31 (s, 1H), 6.09-6.02 (m, 1H), 4.12 (s, 2H), 2.92 (s, 3H), 2.42 (s, 3H), 1.76 (d, J=6.8 Hz, 3H). LCMS (ESI): m/z=442 [M+H]⁺.

### Example 23: (R)—N-(1-(2-(1H-1,2,3-triazol-1-yl)quinolin-4-yl)ethyl)-5-(aminomethyl)-2-methylbenzamide

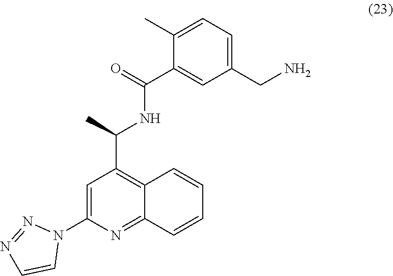 [SMILES: Cc1ccc(CN)cc1C(=O)N[C@H](C)c1cc(-n2ccnn2)nc2ccccc12]

### Step 1. Preparation of (R)—N-(l-(2-(1H-1,2,3-triazol-1-yl)quinolin-4-yl)ethyl)-5-cyano-2-methylbenzamide

To a solution of (R)-5-cyano-2-methyl-N-(1-(quinolin-4-yl)ethyl)benzamide (0.45 g, 1.43 mmol) in MeCN (4.5 mL) were added 1H-triazole (2.02 g, 5.71 mmol) and 1-chloromethyl-4-fluoro-1,4-diazoniabicyclo[2.2.2]octane bis(tetrafluoroborate) (Selectfluor) (443.47 mg, 6.42 mmol) at 25° C., then the reaction was stirred at 60° C. for 12 hrs. LCMS showed the starting material was consumed and a product with desired mass was detected. The mixture was diluted with water (15 mL) and extracted with ethyl acetate (3×10 mL). The combined organic layer was washed with brine (15 mL), dried over sodium sulfate, filtered and concentrated under reduced pressure to give a residue, which was purified by prep-HPLC (column: Phenomenex Luna 80*30 mm*3 μm, Condition water (HCl)-MeCN, Begin: B 30, End: B 50, Gradient Time (min) 8, 100% B Hold Time (min): 2, FlowRate (mL/min): 25) to yield (R)—N-(1-(2-(1H-1,2,3-triazol-1-yl)quinolin-4-yl)ethyl)-5-cyano-2-methylbenzamide (80 mg, 32%) as a yellow solid. ¹H NMR (400 MHz, DMSO-d6) δ1.66 (d, J=7.00 Hz, 3H), 2.36 (s, 3H), 5.87-6.07 (m, 1H), 7.51 (d, J=8.00 Hz, 1H), 7.73-7.98 (m, 4H), 8.03-8.17 (m, 2H), 8.37-8.50 (m, 2H), 9.06 (d, J=1.25 Hz, 1H), 9.41 (d, J=7.38 Hz, 1H). LCMS (ESI): m/z=383.2 [M+H]⁺.

### Step 2. Preparation of (R)—N-(1-(2-(1H-1,2,3-triazol-1-yl)quinolin-4-yl)ethyl)-5-(aminomethyl)-2-methylbenzamide

To a solution of (R)—N-(1-(2-(1H-1,2,3-triazol-1-yl)quinolin-4-yl)ethyl)-5-cyano-2-methylbenzamide (80 mg, 209.20 μmol) and Raney Ni (35.85 mg, 418.39 μmol) in i-PrOH (5 mL) and THF (4 mL) was added NH₄OH (1 mL), then the reaction was stirred at 20° C. for 12 hrs under H₂ (15 psi, i.e. 1,03421 bar). TLC (PE/EA=1/1) showed starting material was consumed and a product with a desired mass was detected. The reaction mixture was filtered and filtrate was diluted with water (20 mL) and extracted with ethyl acetate (3×10 mL). The combined organic layer was washed with brine (15 mL), dried over Na₂SO₄, filtered and concentrated under reduced pressure to give a crude product, which was purified by prep-HPLC (column: Phenomenex Luna 80*30 mm*3 μm, condition water (HCl)-acetonitrile, Begin: B 5, End: B 35, Gradient Time (min) 8, 100% B Hold Time (min): 2, FlowRate (mL/min): 25) to yield (R)—N-(1-(2-(1H-1,2,3-triazol-1-yl)quinolin-4-yl)ethyl)-5-(aminomethyl)-2-methylbenzamide (15 mg, 18.5%) as a white solid. ¹H NMR (400 MHz, DMSO-d₆) δ1.63 (br d, J=6.88 Hz, 3H), 2.30 (s, 3H), 4.02 (br d, J=5.25 Hz, 2H), 5.97 (br t, J=7.07 Hz, 1H), 7.30 (br d, J=7.88 Hz, 1H), 746 (br d, J=7.75 Hz, 1H), 7.55 (s, 1H), 7.74 (br t, J=7.38 Hz, 1H), 7.90 (br t, J=7.57 Hz, 1H), 8.08 (d, J=8.38 Hz, 1H), 8.21 (s, 1H), 8.37-8.44 (m, 4H), 9.26 (br d, J=7.25 Hz, 1H), 9.56 (s, 1H). LCMS (ESI): m/z=387.2 [M+H]⁺.

The following examples were prepared in a similar manner to Example 23 using appropriate reactants.

| Ex. | Structure; | Analogous method, | |
|---|---|---|---|
| # | Compound Name | reactants | Characterizing Data |
| 24 | 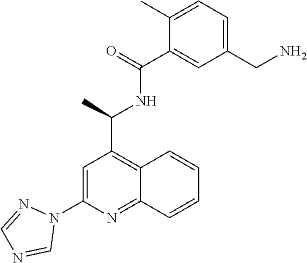 [SMILES: Cc1ccc(CN)cc1C(=O)N[C@H](C)c1cc(-n2cncn2)nc2ccccc12] | Example 23, Step 1, (R)- 5-cyano-2-methyl-N-(1- (quinolin-4-yl)ethyl) benzamide and 4H- 1,2,4-triazole; then Example 23, Step 2, (R)-N-(1-(2-(1H-1,2,4- triazol-1-yl)quinolin-4- yl)ethyl)-5-cyano-2- methylbenzamide | ¹H NMR (400 MHz, DMSO- d6) δ 1.65 (br d, J = 6.00 Hz, 3H), 2.30 (br s, 3H), 4.02 (br s, 2H), 6.01 (br d, J = 6.13 Hz, 1H), 7.30 (br d, J = 7.00 Hz, 1H), 7.47 (br d, J = 6.00 Hz, 1H), 7.58 (br s, 1H), 7.78 (br d, J = 6.63 Hz, 1H), 7.92 (br s, 1H), 8.02- 8.17 (m, 2H), 8.45 (br s, 4H), 9.05 (br s, 1H), 9.29 (br d, J = 5.88 Hz, 1H). LCMS (ESI): m/z = 387.2 [M + H]⁺. |
| | (R)-N-(1-(2-(1H-1,2,4- | | |
| | triazol-1-yl)quinolin-4- | | |
| | yl)ethyl)-5-(aminomethyl)-2- | | |
| | methylbenzamide | | |
| 33 | 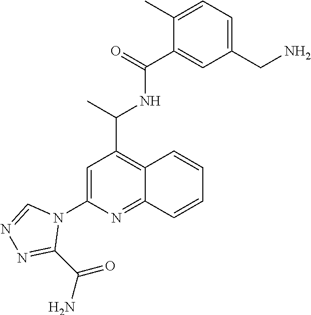 [SMILES: Cc1ccc(CN)cc1C(=O)NC(C)c1cc(-n2cnnc2C(N)=O)nc2ccccc12] | Example 23, Step 1, 5- cyano-2-methyl-N-(1- (quinolin-4-yl)ethyl) benzamide and 1H- 1,2,4-triazole-5- carboxamide; then Example 23, Step 2, 4-(4-(1-(5-cyano-2- methylbenzamido)ethyl) quinolin-2-yl)-4H-1,2,4- triazole-3-carboxamide | ¹H NMR (400 MHz, CD3OD, 298 K) δ (ppm) = 9.63-9.56 (m, 1H), 8.45- 8.40 (m, 1H), 8.38-8.32 (m, 1H), 8.21-8.14 (m, 1H), 7.95-7.86 (m, 1H), 7.82- 7.72 (m, 1H), 7.51-7.43 (m, 1H), 7.41-7.34 (m, 1H), 7.32-7.23 (m, 1H), 6.21- 6.04 (m, 1H), 3.92-3.86 (m, 2H), 2.38 (s, 3H), 1.82-1.74 (m, 3H). LCMS (ESI): m/z = 430.4 [M + H]⁺. |
| | 4-(4-(1-(5-(aminomethyl)-2- | | |
| | methylbenzamido)ethyl) | | |
| | quinolin-2-yl)-4H-1,2,4- | | |
| | triazole-3-carboxamide | | |

### Example 25: (R)—N-(3-(((RS)-1-(2-aminoquinolin-4-yl)ethyl)carbamoyl)-4-methylphenyl) piperidine-2-carboxamide

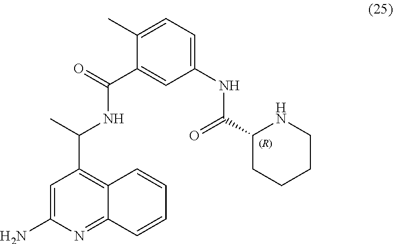

### Step 1. Preparation of 2-methyl-5-nitro-N-(1 quinolin-4-yl)ethyl)benzamide

To a solution of 2-methyl-5-nitrobenzoic acid (526 mg, 2.90 mmol) in DMF (15.0 mL) were added DIEA (1130 mg, 8.71 mmol), 1-(quinolin-4-yl)ethan-1-amine (500.0 mg, 2.90 mmol) and HATU (1660 mg, 4.35 mmol) at 25° C. The mixture was stirred at 25° C. for 16 h. LCMS showed the starting material was consumed and a desired mass was observed (m/z=336.1 [M+H]⁺. The reaction was then poured into H₂O (15 mL) at 25° C., and the mixture was extracted with ethyl acetate (10 mL×2). The combined organic layer was concentrated in vacuo to give a crude product, which was purified by flash chromatography (12 g silica gel column, EA/PE from 0 to 50%) to give 2-methyl-5-nitro-N-(1-(quinolin-4-yl)ethyl)benzamide (550 mg, 56.5%) as a white solid. ¹H NMR (400 MHz, DMSO-d6) δ=9.32 (br d, J=7.4 Hz, 1H), 8.92 (d, J=4.5 Hz, 1H), 8.32 (d, J=8.4 Hz, 1H), 8.24-8.17 (m, 2H), 8.08 (d, J=8.4 Hz, 1H), 7.81 (t, J=7.5 Hz, 1H), 7.70 (t, J=7.6 Hz, 1H), 7.61-7.54 (m, 2H), 5.92 (quin, J=7.0 Hz, 1H), 2.41 (s, 3H), 1.62 (d, J=7.0 Hz, 3H).

### Step 2. Preparation of 2-methyl-5-nitro-N-(1-(quinolin-4-yl)ethyl)benzamide

To a suspension of 2-methyl-5-nitro-N-(1-(quinolin-4-yl)ethyl)benzamide (550 mg, 1.64 mmol) in MeCN (20.0 mL) was added 1H-1,2,3-triazole (227 mg, 3.28 mmol) and Selectflour (1160 mg, 3.28 mmol) at 25° C., the mixture was stirred at 50° C. for 45 h. LCMS showed 50.3% of starting material remained and 44.0% of desired product formed. The reaction was stirred at 60° C. for a further 18 h. LCMS showed 42.6% of starting material remained and 52.0% of desired product formed. The reaction mixture was concentrated in vacuo then partitioned between ethyl acetate (30 mL) and H₂O (30 mL). The layers were separated, and the aqueous phase was extracted with ethyl acetate (10 mL×3). The combined organic layer was concentrated and purified by flash chromatography (12 g silica gel column, EA/PE from 0 to 50%) to give N-(1-(2-(1H-1,2,3-triazol-1-yl)quinolin-4-yl)ethyl)-2-methyl-5-nitrobenzamide (210 mg, 23.6%) and 2-methyl-5-nitro-N-(1-(quinolin-4-yl)ethyl)benzamide (280 mg) as white solids.

N-(1-(2-(1H-1,2,3-triazol-1-yl)quinolin-4-yl)ethyl)-2-methyl-5-nitrobenzamide. ¹H NMR (400 MHz, DMSO-dc) δ=9.54 (d, J=7.3 Hz, 1H), 9.06 (s, 1H), 8.46-8.41 (m, 2H), 8.29-8.20 (m, 2H), 8.13 (d, J=7.5 Hz, 1H), 810-8.04 (m, 1H), 7.94 (t, J=7.6 Hz, 1H), 7.87-7.68 (m, 1H), 7.60 (d, J=9.2 Hz, 1H), 6.01 (t, J=7.0 Hz, 1H), 2.41 (s, 3H), 1.68 (d, J=7.1 Hz, 3H).

### Step 3. Preparation of N-(1-(2-(1H-1,2,3-triazol-1-yl)quinolin-4-yl)ethyl)-5-amino-2-methylbenzamide

To a suspension of wet Pd/C (100 mg 0.094 mmol) in ethyl acetate (10 mL) was added a solution of 2-methyl-5-nitro-N-(1-(quinolin-4-yl)ethyl)benzamide (210 mg, 0.522 mmol) in ethyl acetate (10 mL). The mixture was degassed and refilled with H₂ three times, then stirred at 30° C. under 15 psi (1.03421 bar) for 16 h. TLC (PE/EA=2/1, UV) showed the starting material was consumed and one new main spot was observed. The reaction mixture was filtered and the cake was washed with ethyl acetate (10 mL×3). The filtrate was concentrated in vacuo to give N-(1-(2-(1H-1,2,3-triazol-1-yl)quinolin-4-yl)ethyl)-5-amino-2-methylbenzamide (200 mg, 103%) as a colorless gum. LCMS m/z=3731, [M+H]⁺.

### Step 4. Preparation of tert-butyl(2R)-2-((3-(1-(2-(1H-1,23-triazol-1-yl)quinolin-4-yl)ethyl) carbamoyl)-4-methylphenyl)carbamoyl)piperidine-1-carboxylate

To a solution of (R)-1-(tert-butoxycarbonyl)piperidine-2-carboxylic acid (123 mg, 0.537 mmol) in DMF (5.0 mL) were added DIEA (208 mg, 1.61 mmol), N-(1-(2-(1H-1,2,3-triazol-1-yl)quinolin-4-yl)ethyl)-5-amino-2-methylbenzamide (200 mg, 0.537 mmol) and HATU (306 mg, 0.806 mmol) at 30° C. The mixture was stirred at 30° C. for 16 h. LCMS showed that the starting material was consumed and the desired product formed (61.931%). The reaction mixture was poured into ice water (20 mL) and filtered. The filter cake was dried in vacuo to give tert-butyl(2R)-2-((3-((1-(2-(1H-1,2,3-triazol-1-yl)quinolin-4-yl)ethyl)carbamoyl)-4-methylphenyl)carbamoyl)piperidine-1-carboxylate (300 mg, 95.7%) as a white solid. LCMS m/z=584.2 [M+H].

### Step 5. Preparation of (2R)—N-(3-((1-(2-amino uinolin-4-yl)ethyl)carbamoyl)-4-methylphenyl) piperidine-2-carboxamide

To a solution of tert-butyl(2R)-2-((3-((1-(2-(1H-1,2,3-triazol-1-yl)quinolin-4-yl)ethyl)carbamoyl)-4-methylphenyl)carbamoyl)piperidine-1-carboxylate (300 mg, 0.514 mmol) in dichloromethane (10.0 mL) was added 4N HCl/dioxane (1500 mg, 40 mmol) at 25° C., then the reaction mixture was stirred at 25° C. for 16 h. LCMS showed the reaction produced (2R)—N-(3-((1-(2-aminoquinolin-4-yl)ethyl)carbamoyl)-4-methylphenyl)piperidine-2-carboxamide. The reaction mixture was concentrated in vacuo and purified by prep-HPLC to afford (2R)—N-(3-((1-(2-aminoquinoline-4-yl)ethyl)carbamoyl)-4-methylphenyl)piperidine-2-carboxamide (61.84 mg, 28%) as a white solid. ¹H NMR (400 MHz, DMSO-dr) 0=9.64 (br s, 1H), 8.84 (d, J=7.8 Hz, 1H), 7.94 (d, J=8.4 Hz, 1H), 7.64 (d, J=7.9 Hz, 1H), 7.61 (s, 1H), 7.52-7.45 (m, 2H), 7.22-7.12 (m, 2H), 6.81 (s, 1H), 6.41 (s, 2H), 5.67 (br t, J=7.0 Hz, 1H), 3.32-3.29 (m, 3H), 3.21 (br d, J=6.7 Hz, 1H), 2.95 (br d, J=12.7 Hz, 1H), 2.71-2.61 (m, 1H), 2.58 (br s, 1H), 2.47-2.29 (m, 2H), 2.23 (s, 3H), 1.77 (br s, 2H), 1.59-1.44 (m, 5H), 1.44-1.29 (m, 3H). ¹H NMR (400 MHz, DMSO-d₆+D₂O) δ=7.93 (d, J=8.3 Hz, 1H), 7.61 (s, 1H), 7.60 (d, J=7.8 Hz, 1H), 7.49 (d, J=3.5 Hz, 2H), 7.22-7.19 (m, 1H), 7.15 (d, J=7.8 Hz, 1H), 6.82 (s, 1H), 5.65 (q, J=6.5 Hz, 1H), 3.58-3.55 (m, 1H), 3.50-3.33 (m, 1H), 3.24-3.17 (m, 1H), 2.94 (br d, J=12.9 Hz, 1H), 2.22 (s, 3H), 1.83-1.69 (m, 2H), 1.55-1.30 (m, 7H). LCMS m/z=432.4 [M+H]⁺.

### Prep-HPLC Condition:

Column: Boston Prime C18 150*30 mm*5 μm;
Mobile phase: water (ammonium hydroxide v/v)-acetonitrile; B %: 30%-50%,
Flow Rate (mL/min): 35 mL/min.

### Example 26: (2R)—N-(3-((1-(2-(1H-1,2,3-triazol-1-yl)quinolin-4-yl)ethyl)carbamoyl)-4-methylphenyl)piperidine-2-carboxamide

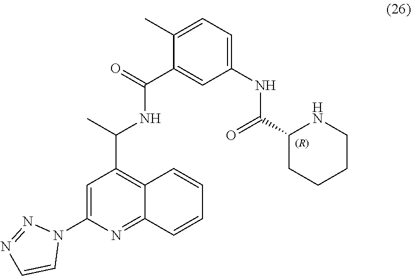

To a solution of tert-butyl(2R)-2-((3-((1-(2-(1H-1,2,3-triazol-1-yl)quinolin-4-yl)ethyl)carbamoyl)-4-methylphenyl)carbamoyl)piperidine-1-carboxylate (140 mg, 0.240 mmol) in dichloromethane (10 mL) was added TFA (137 mg, 1.20 mmol) at 30° C. The mixture was then stirred at 30° C. for 2 h. LCMS showed 78.6% of starting material remained, and no desired product was observed. Additional TFA (137 mg, 1.20 mmol) was added to the mixture and stirred at 30° C. for 5.5 h. LCMS showed 26.6% of starting material remained and 62.4% of the desired product was observed. The reaction mixture was stored at 5° C. for 18 h. LCMS showed 18.3% of starting material remained and 61.6% of the desired product was observed. The reaction mixture was warmed to 30° C. for 2 h. LCMS showed 15.8% of starting material remained and 69.7% of the desired product was observed. Additional TFA (100 mg, 0.877 mmol) was added to the mixture and stirred at 30° C. for 4 h. LCMS showed 5.9% of starting material remained and 78.7% of the desired product was observed. To the reaction mixture was added NH₃ in MeOH (7N) at 0° C. until pH=7-8. The mixture was filtered, and the filtrate was concentrated in vacuo to give (2R)—N-(3-((1-(2-(1H-1,2,3-triazol-1-yl)quinolin-4-yl)ethyl)carbamoyl)-4-methylphenyl)piperidine-2-carboxamide (100 mg, 86.2%) as a brown gum. LCMS (ESI): m/z=484.2 [M+H]⁺.

### Example 27: (R)—N-(3-(((R)-1-(2-aminoquinolin-4-yl)ethyl)carbamoyl)-4-methylphenyl) piperidine-2-carboxamide, ENT-1

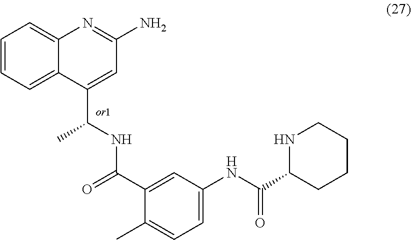 [SMILES: Cc1ccc(NC(=O)[C@H]2CCCCN2)cc1C(=O)N[C@H](C)c1cc(N)nc2ccccc12]

(R)—N-(3-(((RS)-1-(2-aminoquinolin-4-yl)ethyl)carbamoyl)-4-methylphenyl)piperidine-2-carboxamide (54.8 mg, 0.127 mmol, Example 25) was purified by SFC to afford crude-1 (15 mg, first peak) and crude-2 (15 mg, second peak) as white solids.

### Prep-SFC Condition:

Column: DAICEL CHIRALPAK IC (250 mm*30 mm, 10 μm);
Mobile phase: 0.1% NH3H2O ETOH; B %: 60%-60%,
Flow Rate (mL/min): 80 mL/min.

Crude-1 was further purified by prep-HPLC to afford pure (R)—N-(3-(((R*)-1-(2-aminoquinolin-4-yl)ethyl)carbamoyl)-4-methylphenyl)piperidine-2-carboxamide, ENT-1 (6.77 mg, 42%, HCl salt) as a white solid. ¹H NMR (400 MHz, MeOD-d₄) δ=8.28 (d, J=8.3 Hz, 1H), 7.88-7.82 (m, 1H), 7.78-7.61 (m, 4H), 7.26 (s, 1H), 7.27 (d, J=8.5 Hz, 1H), 5.84 (q, J=7.0 Hz, 1H), 4.05-3.99 (m, 1H), 3.47 (br d, J=12.5 Hz, 1H), 3.16-3.07 (m, 1H), 2.45-2.35 (m, 4H), 2.04-1.89 (m, 2H), 1.85-1.67 (m, 6H). LCMS m/z=432.3, M+H, 97.3% purity. SFC ee=99.66%.

### Prep-HPLC Condition:

Column: Boston Green ODS 150*30 mm*5 μm;
Mobile phase: water (HCl)-acetonitrile; B %: 1%-41%,
Flow Rate (mL/min): 30 mL/min.

### Example 28: (R)—N-(3-(((R*)-1-(2-aminoquinolin-4-yl)ethyl)carbamoyl)-4-methylphenyl) piperidine-2-carboxamide, ENT-2

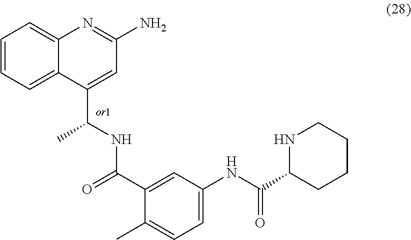 [SMILES: Cc1ccc(NC(=O)[C@H]2CCCCN2)cc1C(=O)N[C@H](C)c1cc(N)nc2ccccc12]

Crude-2 (prepared in Example 27) was further purified by prep-HPLC using the same protocol in Example 27 to afford pure (R)—N-(3-(((R*)-1-(2-aminoquinalin-4-yl)ethyl)carbamayl)-4-methylphenyl)piperidine-2-carboxamide, ENT-2 (6.37 mg, 39.2%, HCl salt) as a white solid. ¹H NMR (400 MHz, MeOD-d₄) δ=8.28 (d, J=8.1 Hz, 1H), 7.87-7.61 (m, 5H), 7.29-7.24 (m, 2H), 5.87-5.80 (m, 1H), 4.04-3.97 (m, 1H), 3.47 (br d, J=12.5 Hz, 1H), 3.16-3.07 (m, 1H), 2.41-2.34 (m, 4H), 2.06-1.90 (m, 2H), 1.86-1.66 (m, 5H). LCMS m/z=432.3 [M+H]⁺, 97.4% purity. SFC ee=99.1%.

### Example 29: (R)—N-(3-(((R*)-1-(2-(1H-1,2,3-triazol-1-yl)quinolin-4-yl)ethyl)carbamoyl)-4-methylphenyl)piperidine-2-carboxamide, ENT-1

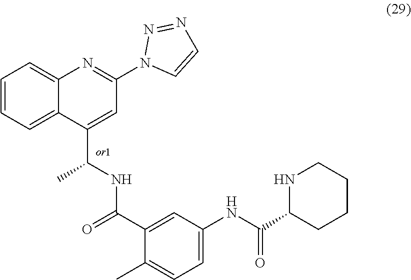 [SMILES: Cc1ccc(NC(=O)[C@H]2CCCCN2)cc1C(=O)N[C@H](C)c1cc(-n2ccnn2)nc2ccccc12]

(R)—N-(3-(((RS)-1-(2-(1H-1,2,3-triazol-1-yl)quinolin-4-yl)ethyl)carbamoyl)-4-methylphenyl)piperidine-2-carboxamide (50 mg) was purified by prep-SFC to afford crude-1 (15 mg, 15%, first peak) and crude-2 (15 mg, 15%, second peak) as white solids.

### Prep-SFC Condition:

Column: DAICEL CHIRALPAK AD (250 mm*30 mm, 10 μm);
Mobile phase: 0.1% NH₃·H₂O EtOH; B %: 45%-45%
Flow Rate (mL/min): 80 mL/min.

Crude-1 was further purified by prep-HPLC to afford (R)—N-(3-(((R*)-1-(2-(1H-1,2,3-triazol-1-yl)quinolin-4-yl)ethyl)carbamoyl)-4-methylphenyl)piperidine-2-carboxamide, ENT-1 (3.92 mg, 26%) as a white solid. ¹H NMR (400 MHz, MeOD-d₄) δ=9.02 (s, 1H), 8.50 (s, 1H), 8.42 (d, J=8.3 Hz, 1H), 8.17 (d, J=8.4 Hz, 1H), 7.99 (s, 1H), 7.89 (t, J=7.2 Hz, 1H), 7.77 (t, J=7.6 Hz, 1H), 7.69 (d, J=2.1 Hz, 1H), 7.52 (dd, J=2, 3, 8.3 Hz, 1H), 7.23 (d, J=8.3 Hz, 1H), 6.10 (q, J=7.0 Hz, 1H), 3.41-3.35 (m, 1H), 3.13 (br d, J=13.4 Hz, 1H), 2.70 (br t, J=12.1 Hz, 1H), 2.34 (s, 3H), 2.01-1.87 (m, 2H), 1.78 (d, J=7.0 Hz, 3H), 1.65 (br d, J=11.0 Hz, 1H), 1.60-1.45 (m, 3H). LCMS (ESI): m/z=484.4 [M+H]⁺, 99% purity, SFC ee=99.7%.

### Prep-HPLC Condition:

Column: Boston Prime C18 150*30 mm*5 μm;
Mobile phase: water (ammonium hydroxide v/v)-acetonitrile; B %: 35%-55%,
Flow Rate (mL/min): 35 mL/min.

### Example 30: (R)—N-(3-(((R*)-1-(2-(1H-1,2,3-triazol-1-yl)quinolin-4-yl)ethyl)carbamoyl)-4-methylphenyl)piperidine-2-carboxamide, ENT-2

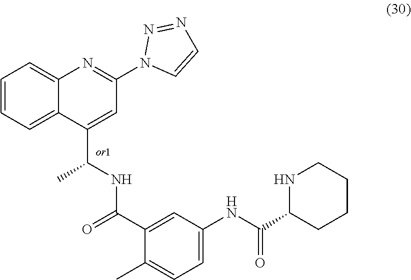 [SMILES: Cc1ccc(NC(=O)[C@H]2CCCCN2)cc1C(=O)N[C@H](C)c1cc(-n2ccnn2)nc2ccccc12]

Crude-2 (prepared in Example 29) was further purified by prep-HPLC, using the same protocol in Example 29, to afford (R)—N-(3-(((R*)-1-(2-(1H-1,2,3-triazol-1-yl)quinolin-4-yl)ethyl)carbamoyl)-4-methylphenyl)piperidine-2-carboxamide, ENT-2 (3.25 mg, 22%) as a white solid. ¹H NMR (400 MHz, MeOD-d₄) δ=9.03 (s, 1H), 8.50 (s, 1H), 8.42 (d, J=8.5 Hz, 1H), 8.17 (d, J=8.4 Hz, 1H), 7.99 (s, 1H), 7.90 (t, J=7.6 Hz, 1H), 7.78 (t, J=7.7 Hz, 1H), 7.69 (d, J=2.1 Hz, 1H), 7.52 (d, J=7.8 Hz, 1H), 7.23 (d, J=8.4 Hz, 1H), 6.10 (q, J=6.9 Hz, 1H), 3.42-3.36 (m, 1H), 3.14 (br d, J=12.9 Hz, 1H), 2.71 (br t, J=12.1 Hz, 1H), 2.34 (s, 3H), 1.98 (br d, J=9.3 Hz, 1H), 1.92 (br s, 1H), 1.78 (d, J=7.0 Hz, 3H), 1.66 (br d, J=10.8 Hz, 1H), 1.61-1.47 (in, 3H). LCMS (ESI): m/z=484.4 [M+H], 99.5% purity. SFC ee=98.1%.

### Example 31: 5-(aminomethyl)-2-methyl-N-(1-(2-(4-(trifluoromethyl)-1H-1,2,3-triazol-1-yl)quinolin-4-yl)ethyl)benzamide

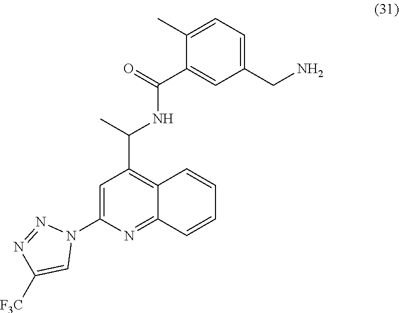 [SMILES: Cc1ccc(CN)cc1C(=O)NC(C)c1cc(-n2cc(C(F)(F)F)nn2)nc2ccccc12]

### Step 1. Preparation of 5-cyano-2-methyl-N(1-(2-(4-(trifluoromethyl)-1H-1,2,3-triazol-1-yl)quinolin-4-yl)ethyl)benzamide

To a suspension of 5-cyano-2-methyl-N-(1-(quinolin-4-yl)ethyl)benzamide (120 mg, 0.381 m mol) in MeCN (4.0 mL) was added 5-(trifluoromethyl)-1H-1,2,3-triazole (120 mg, 0.875 mmol) and Selectfluor (270 mg, 0.761 mmol) at 25° C. The mixture was stirred at 60° C. for 12 h. LCMS showed that a desired mass was produced. The reaction was filtered. The filtrate was concentrated in vacuo and purified by prep-HPLC to afford 5-cyano-2-methyl-N-(1-(2-(4-(trifluoromethyl)-1H-1,2,3-triazol-1-yl)quinolin-4-yl)ethyl)benzamide (5 mg, 2.92%) and 5-cyano-2-methyl-N-(1-(2-(4-(trifluoromethyl)-2H-1,2,3-triazol-2-yl)quinolin-4-yl)ethyl)benzamide (12 mg, 7%) as white solids after lyophilization. LCMS (ESI): m/z=451.2 [M+H]⁺ (for both).

### Prep-HPLC Method:

Mobile phase: A: water (0.05% ammonium hydroxide v/v); B: acetonitrile
Column: Boston Prime C18 150*30 mm*5 μm
Flow rate: 30 mL/min
Monitor wavelength: 220&254 nm
Gradient: B %, 30% to 70%, 3 min, 100% B Hold Time (min) 2 min

### Step 2. Preparation of 5-(aminomethyl)-2-methyl-N-(1-(2-(4-(trifluoromethyl)-1H-1,2,3-triazol-1-yl)quinolin-4-yl)ethyl)benzamide

To a solution of Raney-Ni (2.66 mg, 0.0311 mmol) in THF (2.0 mL) and IPA (2.0 mL) was added 5-cyano-2-methyl-N-(1-(2-(4-(trifluoromethyl)-1H-1,2,3-triazol-1-yl)quinolin-4-yl)ethyl)benzamide (5.0 mg, 0.01 mmol). The reaction was degassed and refilled with H₂ (3 times) then stirred at 20° C. and 20 psi (1.37895 bar) for 16 h. LCMS showed a desired mass. Following filtration, the filtrate was concentrated in vacuo and purified by prep-HPLC to afford 5-(aminomethyl)-2-methyl-N-(1-(2-(4-(trifluoromethyl)-1H-1,2,3-triazol-1-yl)quinolin-4-yl)ethyl)benzamide(1.52 mg, 30%) as a white solid after lyophilization. ¹H NMR (400 MHz, CD3OD, 296 K) δ(ppm)=9.59-9.48 (m, 1H), 8.57-8.51 (m, 1H), 8.48-8.40 (m, 1H), 8.24-8.18 (m, 1H), 7.97-7.90 (m, 1H), 7.85-7.76 (m, 1H), 7.48-7.43 (m, 1H), 7.41-7.33 (m, 1H), 7.31-7.21 (m, 1H), 6.21-5.96 (m, 1H), 3.86 (s, 2H), 2.38 (s, 3H), 1.78 (d, J=7.0 Hz, 3H). LCMS (ESI): m/z=455.4 [M+H]⁺.

### Prep-HPLC Method:

Mobile phase: A: water (0.05% ammonium hydroxide v/v); B: acetonitrile
Column: C18 150*30 mm*5 μm
Flow rate: 30 mL/min
Monitor wavelength: 220&254 nm
Gradient: B %, 31% to 71%, 9 min; 100%, 2 min

### Example 32: 5-(aminomethyl)-2-methyl-N-(1-(2-(4-(trifluoromethyl)-2H-1,2,3-triazol-2-yl)quinolin-4-yl)ethyl)benzamide

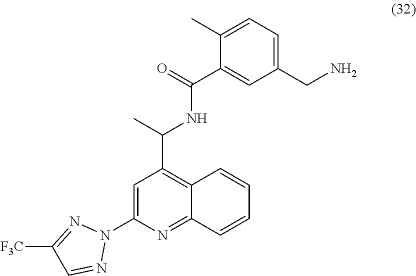 [SMILES: Cc1ccc(CN)cc1C(=O)NC(C)c1cc(-n2ncc(C(F)(F)F)n2)nc2ccccc12]

Using a procedure analogous to Example 31 Step 2 with 5-cyano-2-methyl-N-(1-(2-(4-(trifluoromethyl)-2H-1,2,3-triazol-2-yl)quinolin-4-yl)ethyl)benzamide (5.0 mg, 0.01 mmol) as the reactant affords 5-(aminomethyl)-2-methyl-N-(1-(2-(4-(trifluoromethyl)-2H-1,2,3-triazol-2-yl)quinolin-4-yl)ethyl)benzamide (2.51 mg, 21%) as a white solid. ¹H NMR (400 MHz, CD3OD, 296 K) δ (ppm)=8.58 (s, 1H), 8.51-8.48 (m, 1H), 8.48-8.41 (m, 1H), 8.32-8.22 (m, 1H), 8.01-7.90 (m, 1H), 7.86-7.70 (m, 1H), 7.45-7.33 (m, 2H), 7.31-7.23 (m, 1H), 6.11 (q, J=7.0 Hz, 1H), 3.85 (s, 2H), 2.38 (s, 3H), 1.78 (d, J=7.0 Hz, 3H). LCMS (ESI): m/z=455.3 [M+H]⁺.

### Example 34: 5-(aminomethyl)-2-methyl-N-(1-(2-(2-oxopyrrolidin-1-yl)quinolin-4-yl)ethyl)benzamide

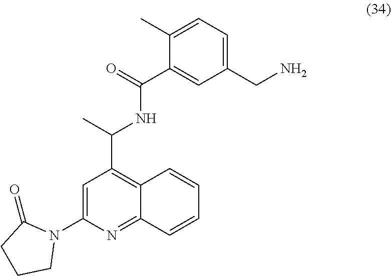 [SMILES: Cc1ccc(CN)cc1C(=O)NC(C)c1cc(N2CCCC2=O)nc2ccccc12]

### Step 1. Preparation of tert-butyl(4-methyl-3-((1-(2-(2-oxopyrrolidin-1-yl)quinolin-4-yl)ethyl carbamoyl)benzyl carbamate

A mixture of tert-butyl(3-((1-(2-chloroquinolin-4-yl)ethyl)carbamoyl)-4-methylbenzyl)carbamate (P7) (80.0 mg, 0.18 mmol), pyrrolidin-2-one (45.0 mg, 0.529 mmol), Pd₂(dba)₃ (16.1 mg, 0.0176 mmol), K₂CO₃ (73.1 mg, 0.529 mmol), and x-phos (8.40 mg, 0.0176 mmol) in dioxane (5.00 mL) was degassed with N₂. The brown reaction mixture was stirred at 100° C. for 12 h. LCMS showed a peak having desired mass. The reaction was filtered. The filtrate was concentrated in vacuo to give tert-butyl(4-methyl-3-((1-(2-(2-oxopyrrolidin-1-yl)quinolin-4-yl)ethyl)carbamoyl)benzyl)carbamate (89 mg, 100%) as a brown oil. LCMS (ESI): m/z=503.2 [M+H]⁺.

### Step 2. Preparation of 5-(aminomethyl)-2-methyl-N-(1-(2-(2-oxopyrrolidin-1-yl)quinolin-4-yl)ethyl)benzamide

To a flask of tert-butyl(4-methyl-3-((1-(2-(2-oxopyrrolidin-1-yl)quinolin-4-yl)ethyl) carbamoyl)benzyl)carbamate (89 mg, 0.18 mmol) was added 4M HCl/dioxane (5 mL). The solution was stirred at 25° C. for 1 h. Then the solution was concentrated to get a crude product, which was dissolved in CH₃OH (2 mL) and purified by prep-HPLC to afford 5-(aminomethyl)-2-methyl-N-(1-(2-(2-oxopyrrolidin-1-yl)quinolin-4-yl)ethyl)benzamide (9.85 mg, 14%) as a white solid after lyophilization. ¹H NMR (400 MHz, CDCl₃, 303 K) δ (ppm)=881 (s, 1H), 8.22-8.11 (m, 1H), 8.00-7.90 (m, 1H), 7.75-7.63 (m, 1H), 7.61-7.50 (m, 2H), 6.71-6.55 (m, 1H), 6.20-6.05 (m, 1H), 4.39-4.19 (m, 2H), 3.97-3.79 (m, 2H), 2.83-2.65 (m, 2H), 2.48-2.42 (m, 1H), 2.51-2.38 (m, 2H), 2.26-213 (m, 2H), 1.83-1.77 (m, 3H). LCMS (ESI): m/z=403.4 [M+H]⁺.

### Prep-HPLC Method:

Mobile phase: A: water (0.05% ammonium hydroxide v/v); B: acetonitrile
Column: C18 150*30 mm*5 μm
Flow rate: 30 mL/min
Monitor wavelength: 220&254 nm
Gradient: B %, 19% to 59%, 9 min, 100% B Hold Time (min) 2 min

### Example 35: 5-(aminomethyl)-N-(1-(2-(3-cyano-1H-pyrazol-1-yl)quinolin-4-yl)ethyl)-2-methylbenzamide

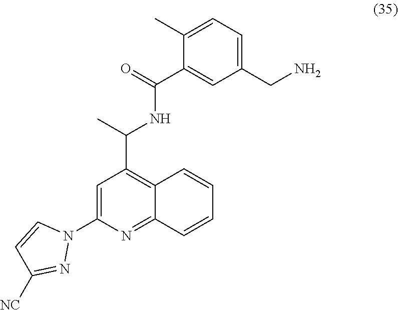 [SMILES: Cc1ccc(CN)cc1C(=O)NC(C)c1cc(-n2ccc(C#N)n2)nc2ccccc12]

### Step 1. Preparation of tert-butyl(3-((1-(2-(3-cyano-1H-pyrazol-1-yl)quinolin-4-yl)ethyl carbamoyl)-4-methylbenzyl)carbamate

Using a procedure analogous to Example 1 Step 2 with tert-butyl(3-((1-(2-chloroquinolin-4-yl)ethyl)carbamoyl)-4-methylbenzyl)carbamate (80.0 mg, 0.18 mmol) and 1H-pyrazole-3-carbonitrile (57.4 mg, 0.617 mmol) as the reactants affords tert-butyl(3-((1-(2-(3-cyano-1H-pyrazol-1-yl)quinolin-4-yl)ethyl)carbamoyl)-4-methylbenzyl)carbamate (50 mg, 56%) as a brown oil. LCMS (ESI): m/z=455.1 [M-tBu]⁺, 533.1 [M+Na]⁺.

### Step 2. Preparation of 5-(aminomethyl)-N-(1-(2-(3-cyano-1H-pyrazol-1-yl)quinolin-4-yl)ethyl)-2-methylbenzamide

Using a procedure analogous to Example 34 Step 2 with tert-butyl(3-((1-(2-(3-cyano-1H-pyrazol-1-yl)quinolin-4-yl)ethyl)carbamoyl)-4-methylbenzyl)carbamate (50 mg, 0.098 mmol) as the reactant affords 5-(aminomethyl)-N-(1-(2-(3-cyano-1H-pyrazol-1-yl)quinolin-4-yl)ethyl)-2-methylbenzamide (3.3 mg, 8.2%) as a white solid. ¹H NMR (400 MHz, DMSO-dF, 297 K) δ (ppm)=9.25-9.18 (m, 1H), 9.11-9.05 (m, 1H), 8.47-8.39 (m, 1H), 8.35-8.29 (m, 1H), 8.12-8.08 (m, 1H), 7.95-7.89 (m, 1H), 7.81-7.71 (m, 1H), 7.41-7.32 (m, 3H), 7.25-7.20 (m, 1H), 6.01-5.89 (m, 1H), 3.85-3.70 (m, 2H), 2.29-2.25 (m, 3H), 1.62 (br d, J=7.0 Hz, 3H). LCMS (ESI): m/z=411.3 [M+H]⁺.

The following example was prepared in a similar manner to Example 35 using appropriate reactants.

| Ex. | Structure; | Analogous method, | |
|---|---|---|---|
| # | Compound Name | reactants | Characterizing Data |
| 36 | 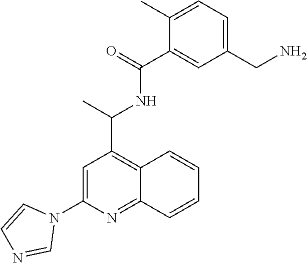 [SMILES: Cc1ccc(CN)cc1C(=O)NC(C)c1cc(-n2ccnc2)nc2ccccc12] | Example 35, Step 1, tert- butyl (3-((1-(2- chloroquinolin-4- yl)ethyl)carbamoyl)-4- methylbenzyl)carbamate and 1H-imidazole; then Example 35, Step 2, tert-butyl (3-((1-(2-(1H- imidazol-1-yl)quinolin-4- yl)ethyl)carbamoyl)-4- methylbenzyl)carbamate | ¹H NMR (400 MHz, CD3OD, 297 K) δ (ppm) = 10.09 (s, 1H), 9.13 (br d, J = 7.6 Hz, 1H), 8.66 (s, 1H), 8.51 (d, J = 8.3 Hz, 1H), 8.27-8.18 (m, 2H), 8.00- 7.92 (m, 1H), 7.89-7.78 (m, 2H), 7.66-7.57 (m, 1H), 7.50-7.42 (m, 1H), 7.41- 7.30 (m, 1H), 6.23-6.11 (m, 1H), 4.14 (s, 2H), 2.37 (s, 3H), 1.86 (d, J = 7.0 Hz, 3H). LCMS (ESI): m/z = 386.3 [M + H]⁺. |
| | N-(1-(2-(1H-imidazol-1- | | |
| | yl)quinolin-4-yl)ethyl)-5- | | |
| | (aminomethyl)-2-methylbenzamide | | |

### Example 37: (R)-2-methyl-N-(1-(2-(oxetan-3-ylamino)quinolin-4-yl)ethyl)benzamide

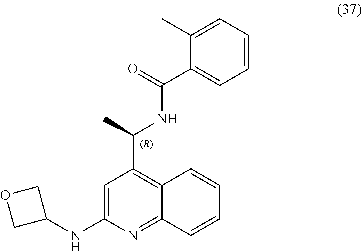

To a solution of (R)-4-(1-(2-methylbenzamido)ethyl)quinoline 1-oxide (P2) (100 mg, 0.32 mmol) in 1,2-dichloroethane (2 mL) were added oxetan-3-amine (31.02 mg, 0.42 mmol), PyBoP (271.78 mg, 0.52 mmol), and DIEA (126.56 mg, 0.98 mmol) at 20° C. The mixture was stirred for 12 h at 80° C. TLC (THF) showed the starting material was consumed and a new spot was detected. The mixture was purified by prep-HPLC (column: Phenomenex Luna 80*30 mm*3 μm, Condition water (HCl)-acetonitrile, Begin B: 5, End B: 35, Gradient Time (m): 8, 100% B Hold Time (min): 2, Flow Rate (mL/min): 25) to yield (R)-2-methyl-N-(1-(2-(oxetan-3-ylamino)quinolin-4-yl)ethyl)benzamide (13.8 mg, 124%) as a yellow solid. ¹H NMR (400 MHz, MeOD-d4) δ1.67 (d, J=7.13 Hz, 3H), 2.39 (s, 3H); 3.72-3.89 (i, 2H), 4.57-4.67 (m, 2H), 5.79-5.90 (m, 3H), 7.20 (d, J=4.25 Hz, 1H), 7.28 (br d, J=7.25 Hz, 2H), 7.34-7.40 (m, 1H), 7.43 (s, 1H), 7.61-7.72 (m, 2H) 7.90-7.99 (m, 1H), 8.34 (d, J=8.25 Hz, H). LCMS (ESI): (m/z=362.1 [M+H]⁺.

The following examples were prepared in a similar manner to Example 37 using (R)-4-(1-(2-methylbenzamido)ethyl)quinoline 1-oxide (2) and an appropriate amine.

| Ex. | Structure; | | |
|---|---|---|---|
| # | Compound Name | Amine | Characterizing Data |
| 38 | 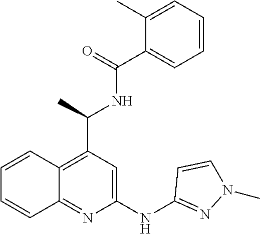 [SMILES: Cc1ccccc1C(=O)N[C@H](C)c1cc(Nc2ccn(C)n2)nc2ccccc12] | 1-methyl- 1H-pyrazol- 3-amine | ¹H NMR (400 MHz, MeOD-d4) δ 1.72 (d, J = 7.09 Hz, 3H), 2.40 (s, 3H), 4.06 (s, 3H), 5.89 (d, J = 7.21 Hz, 1H), 6.21 (d, J = 2.45 Hz, 1H), 7.29 (dd, J = 7.03, 4.58 Hz, 2H), 7.34-7.42 (m, 2H), 7.47- 7.51 (m, 1H), 7.68-7.76 (m, 2H), 7.90- 8.04 (m, 2H), 8.37 (d, J = 8.44 Hz, 1H). LCMS (ESI): m/z = 386.2 [M + H]⁺. |
| | (R)-2-methyl-N-(1-(2-((1- | | |
| | methyl-1H-pyrazol-3- | | |
| | yl)amino)quinolin-4- | | |
| | yl)ethyl)benzamide | | |
| 39 | 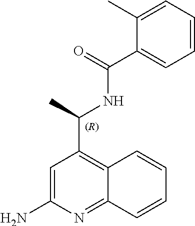 | NH₄Cl | ¹H NMR (400 MHz, MeOD-d4) δ 1.65 (d, J = 6.88 Hz, 3H), 3.10-3.30 (m, 3H), 5.83 (q, J = 6.75 Hz, 1H), 6.93 (s, 1H), 7.19-7.26 (m, 2H), 7.26-7.35 (m, 2H), 7.38 (d, J = 7.63 Hz, 1H), 7.50-7.60 (m, 2H), 8.01 (d, J = 8.25 Hz, 1H). LCMS (ESI): m/z = 306.2 [M + H]⁺. |
| | (R)-N-(1-(2-aminoquinolin-4- | | |
| | yl)ethyl)-2-methylbenzamide | | |
| 41 | 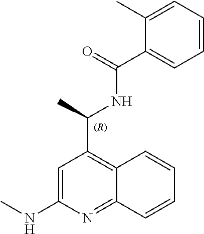 | MeNH₂ | ¹H NMR (400 MHz, MeOD-d₄) δ 1.64 (d, J = 7.00 Hz, 3H), 2.36 (s, 3H), 3.01 (s, 3H), 5.76-5.86 (m, 1H), 6.84 (s, 1H), 7.19-7.28 (m, 3H), 7.29-7.40 (m, 2H), 7.52 (ddd, J = 8.35, 7.04, 1.25 Hz, 1H), 7.67-7.75 (m, 1H), 7.97 (d, J = 7.25 Hz, 1H). LCMS (ESI): m/z = 320.2 [M + H]⁺. |
| | (R)-2-methyl-N-(1-(2- | | |
| | (methylamino)quinolin-4- | | |
| | yl)ethyl)benzamide | | |
| 42 | 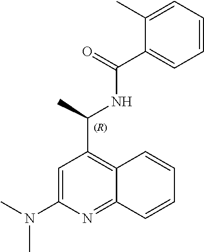 | Me₂NH | ¹H NMR (400 MHz, MeOD-d₄) δ 1.66 (d, J = 7.00 Hz, 3H), 2.37 (s, 3H), 3.23 (s, 6H), 5.89 (d, J = 6.75 Hz, 1H), 7.12 (s, 1H), 7.20-7.40 (m, 5H), 7.54 (br d, J = 1.38 Hz, 1H), 7.71 (d, J = 8.00 Hz, 1H), 8.01 (d, J = 7.63 Hz, 1H). LCMS (ESI): m/z = 334.2 [M + H]⁺. |
| | (R)-N-(1-(2- | | |
| | (dimethylamino)quinolin-4- | | |
| | yl)ethyl)-2-methylbenzamide | | |

### Example 40: (R)N-(1-(2-acetamidoquinolin-4-yl)ethyl)-2-methylbenzamide

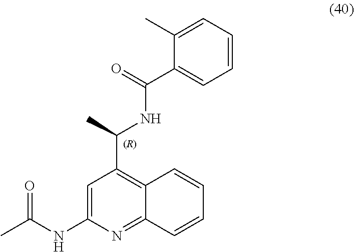

To a solution of (R)—N-(1-(2-aminoquinolin-4-yl)ethyl)-2-methylbenzamide (Example 39) (14 mg, 45.85 μmol) in dichloromethane (1 mL) were added acetic anhydride (14.04 mg, 137.54 μmol), 4-dimethylaminopyridine (0.56 mg, 4.58 μmol), TEA (23.2 mg, 229.23 μmol). The mixture was stirred at 25° C. for 12 h. TLC (PE/EA=1/1) showed that the starting material was consumed and a new spot was detected. The mixture was purified by prep-HPLC to give (R)—N-(1-(2-acetamidoquinolin-4-yl)ethyl)-2-methylbenzamide (2.2 mg, 13.8%) as a white solid. ¹H NMR (400 MHz, MeOD-d4) δ1.67 (d, J=7.03 Hz, 3H), 2.24 (s, 3H), 2.37 (s, 3H), 5.96 (d, J=7.03 Hz, 1H), 7.21-7.29 (m, 2H), 7.30-7.37 (m, 1H), 7.47 (d, J=7.75 Hz, 1H), 7.56 (t, J=7.51 Hz, 1H), 7.66-7.74 (m, 1H), 7.88 (d, J=8.34 Hz, 1H), 8.21 (d, J=8.11 Hz, 1H), 8.49 (s, 1H). LCMS (ESI): m/z=348.2 [M+H]⁺.

### Prep-HPLC Method:

Mobile phase: A: 10 mM NH₄HCO₃ in H₂O; B: ACN
Column: Waters Xbridge BEH C18 100*30 mm*10 μm
Flow rate: 25 mL/min
   Monitor wavelength: 220&254 nm

### Example 43: 2-methyl-N-((1R)-1-(2-((tetrahydrofuran-3-yl)methoxy)quinolin-4-yl)ethyl)benzamide

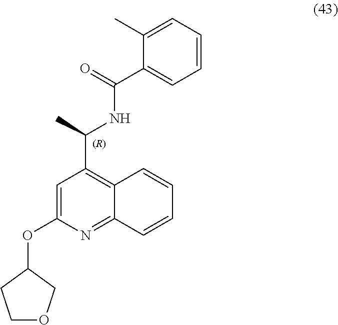

### Preparation of 2-methyl-N-((1R)-1-(2((tetrahydrofuran-3-yl)methoxy)quinolin-4-yl)ethyl)benzamide

To a solution of (tetrahydrofuran-3-yl)methanol (157.22 mg, 1.54 mmol) in anhydrous THF (1 mL) was added NaH (37.00 mg, 1.54 mmol, 60% wt %) portionwise with stirring at 0° C. The reaction was stirred at 0° C. for 1 h. Then a solution of (R)—N-(1-(2-chloroquinolin-4-yl)ethyl)-2-methylbenzamide (50 mg, 0.154 mmol) in anhydrous THF (0.5 mL) was added dropwise at 0° C. The resulting reaction mixture was stirred and heated at 70° C. for 5 h. LCMS showed that the starting material was consumed and a product with a desired mass was detected. The reaction was then quenched with H₂O (2 mL) and extracted with ethyl acetate (3×5 mL). The combined organic layer was dried over Na₂SO₄, filtered, and concentrated under reduced pressure. The crude product was purified by prep-HPLC to give 2-methyl-N-((1R)-1-(2-((tetrahydrofuran-3-yl)methoxy)quinolin-4-yl)ethyl)benzamide (16.16 mg) as a white solid. ¹H NMR (400 MHz, MeOD-d4) δ1.39 (d, J=6.63 Hz, 3H), 1.70 (d, J=7.00 Hz, 3H), 2.34 (s, 3H), 3.60-3.71 (m, 2H), 3.81 (q, J=6.63 Hz, 1H), 6.04 (q, J=6.96 Hz, 1H), 7.12-7.22 (m, 2H), 7.27 (d, J=7.75 Hz, 1H), 7.45-7.59 (m, 3H), 7.63 (d, J=7.13 Hz, 1H), 7.75-7.84 (m, 2H), 7.87-7.94 (m, 1H), 8.15 (s, 1H), 8.25 (d, J=8.51 Hz, 1H). LCMS (ESI): m/z=391.2 [M+H]⁺.

### Prep-HPLC Method:

Mobile phase: A: 10 mM NH₄HCO₃ in H₂O; B:ACN
Column: Waters Xbridge Prep OBD C18 150*40 mm*10 μm
Flow rate: 60 mL/min
Monitor wavelength: 220&254 nm

The following examples were prepared in a similar manner to Example 43 using (R)—N-(1-(2-chloroquinolin-4-yl)ethyl)-2-methylbenzamide (P3) and an appropriate alcohol.

| Ex. | Structure; | | |
|---|---|---|---|
| # | Compound Name | Alcohol | Characterizing Data |
| 44 | 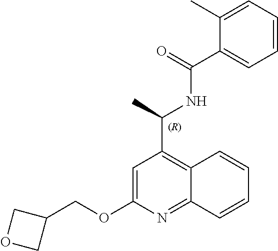 | oxetan-3- ylmethanol | ¹H NMR (400 MHz, MeOD-d4) δ 1.66 (d, J = 7.09 Hz, 3H), 2.35 (s, 3H), 3.45-3.58 (m, 1H), 4.61-4.72 (m, 4H), 4.88-4.92 (m, 2H), 5.92 (q, J = 6.77 Hz, 1H), 7.06 (s, 1H), 7.19-7.28 (m, 2H), 7.30-7.37 (m, 2H), 7.44-7.52 (m, 1H), 7.62-7.70 (m, 1H), 7.86 (d, J = 7.70 Hz, 1H), 8.16 (d, J = 8.31 Hz, 1H). LCMS (ESI): m/z = 377.2 [M + H]⁺. |
| | (R)-2-methyl-N-(1-(2-(oxetan-3- | | |
| | ylmethoxy) quinolin-4-yl)ethyl) | | |
| | benzamide | | |
| 45 | 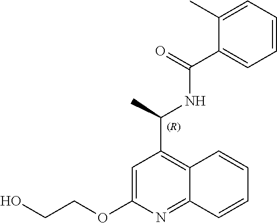 | ethane-1,2- diol | ¹H NMR (400 MHz, MeOD-d4) δ 1.66 (d, J = 6.97 Hz, 3H), 2.36 (s, 3H), 3.94 (dd, J = 5.50, 4.16 Hz, 2H), 4.49-4.59 (m, 2H), 5.92 (q, J = 6.93 Hz, 1H), 7.08 (s, 1H), 7.20- 7.27 (m, 2H), 7.30-7.38 (m, 2H), 7.48 (ddd, J = 8.28, 7.00, 1.22 Hz, 1H), 7.66 (ddd, J = 8.31, 7.03, 1.28 Hz, 1H), 7.80-7.87 (m, 1H), 8.16 (d, J = 7.70 Hz, 1H). LCMS (ESI): m/z = 351.2 [M + H]⁺. |
| | (R)-N-(1-(2-(2- | | |
| | hydroxyethoxy)quinolin-4- | | |
| | yl)ethyl)-2-methylbenzamide | | |
| 46 | 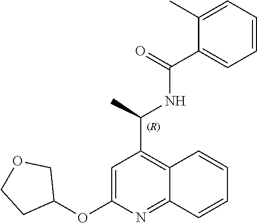 | Tetrahydrofuran- 3-ol | ¹H NMR (400 MHz, CDCl₃) δ 1.72 (dd, J = 6.36, 3.30 Hz, 3H), 2.15- 2.41 (m, 2H), 2.41-2.50 (m, 3H), 3.87-4.08 (m, 3H), 4.13 (dd, J = 10.39, 4.77 Hz, 1H), 5.82 (td, J = 4.34, 2.32 Hz, 1H), 5.93-6.08 (m, 2H), 6.96 (d, J = 2.81 Hz, 1H), 7.13-7.25 (m, 2H), 7.29-7.37 (m, 2H), 7.42-7.51 (m, 1H), 7.61-7.69 (m, 1H), 7.86 (d, J = 7.82 Hz, 1H), 8.09 (br d, J = 8.31 Hz, 1H). LCMS (ESI): m/z = 377.2 [M + H]⁺. |
| | 2-methyl-N-((1R)-1-(2- | | |
| | ((tetrahydrofuran-3- | | |
| | yl)oxy)quinolin-4- | | |
| | yl)ethyl)benzamide | | |
| 47 | 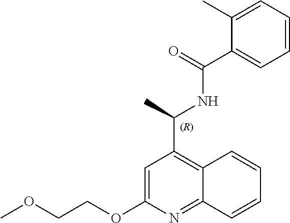 | 2-methoxy ethan-1-ol | ¹H NMR (400 MHz, CDCl₃) δ 1.65- 1.77 (m, 3H), 2.44 (s, 3H), 3.47 (s, 3H), 3.82 (br t, J = 4.52 Hz, 2H), 4.61-4.75 (m, 2H), 5.91-6.10 (m, 2H), 7.04 (s, 1H), 7.15-7.24 (m, 2H), 7.31 (br t, J = 7.83 Hz, 2H), 7.42-7.50 (m, 1H), 7.61-7.70 (m, 1H), 7.87 (br d, J = 8.07 Hz, 1H), 8.09 (br d, J = 8.19 Hz, 1H). LCMS (ESI): m/z = 365.2 [M + H]⁺. |
| | (R)-N-(1-(2-(2- | | |
| | methoxyethoxy)quinolin-4- | | |
| | yl)ethyl)-2-methylbenzamide | | |

### Example 48: (R)—N-(1-(2-(1H-pyrazol-5-yl)quinolin-4-yl)ethyl)-2-methylbenzamide

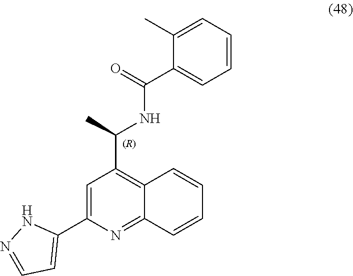

### Step 1. Preparation of 2-methyl-N-((1R)-1-(2-(1-(tetrahydro-2H-pyran-2-yl)-1H-pyrazol-5-yl)quinolin-4-yl)ethyl)benzamide

To a solution of (R)—N-(1-(2-chloroquinolin-4-yl)ethyl)-2-methylbenzamide (60.0 mg, 0.18 mmol) in dioxane (4.0 mL) was added 1-(tetrahydro-2H-pyran-2-yl)-5-(4,4,5,5-tetramethyl-1,3,2-dioxaborolan-2-yl)-1H-pyrazole (154 mg, 0.554 mmol), Pd(dppf)Cl₂ (13.5 mg, 0.0185 mmol), and sat·Na₂CO₃ (0.5 mL) at 20° C. The mixture was bubbled with N₂ for 1 min. Then the mixture was stirred at 100° C. for another 2 h. LCMS showed a mass peak of the desired product. The reaction mixture was partitioned between EA/H₂O (20 mL/10 mL), the organic layer was separated and the aqueous layer was re-extracted with EtOAc (15 mL). The combined organic layer was washed with brine, dried with anhydrous Na₂SO₄, filtered, evaporated in vacuo, and purified by silica gel chromatography via Biotage SP1 (20 g silicon column, ethyl acetate in PE from 0 to 100%) to afford 2-methyl-N-((1R)-1-(2-(1-(tetrahydro-2H-pyran-2-yl)-1H-pyrazol-5-yl)quinolin-4-yl)ethyl)benzamide (68 mg, 84%) as a white solid. LCMS (ESI): m/z=441.2 [M+H]⁺.

### Step 2. Preparation of (R)-N-(1-(2-(1H-pyrazol-5-yl)quinolin-4-yl)ethyl)-2-methylbenzamide

Using a procedure analogous to Example 17, Step 4 with 2-methyl-N-((1R)-1-(2-(1-(tetrahydro-2H-pyran-2-yl)-1H-pyrazol-5-yl)quinolin-4-yl)ethyl)benzamide (68 mg, 0.15 mmol) as the reactant affords (R)—N-(1-(2-(1H-pyrazol-5-yl)quinolin-4-yl)ethyl)-2-methylbenzamide (6.7 mg, 12% yield) was obtained as a white solid. ¹H NMR (400 MHz, DMSO-d6) δ=13.19 (br d, J=2.9 Hz, 1H), 9.13 (br d, J=4.4 Hz, 1H), 8.35-8.24 (m, 2H), 8.06 (d, J=8.3 Hz, 1H), 7.87 (br s, 1H), 7.77 (br t, J=7.3 Hz, 1H), 7.66-7.59 (m, 1H), 7.41-7.32 (m, 2H), 7.29-7.18 (m, 2H), 7.00 (d, J=1.0 Hz, 1H), 5.89 (br t, J=7.3 Hz, 1H), 2.29 (s, 3H), 1.61 (d, J=6.8 Hz, 3H). LCMS (ESI): m/z=357.3 [M+H]⁺.

The following examples were prepared in a similar manner to Example 48 using (R)—N-(1-(2-chloroquinolin-4-yl)ethyl)-2-methylbenzamide (P3) and an appropriate boronic acid or boronic ester.

| Ex. | Structure; | Boronic | |
|---|---|---|---|
| # | Compound Name | acid/boronic ester | Characterizing Data |
| 49 | 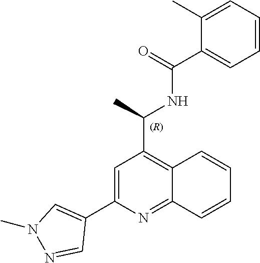 | 1-methyl-4-(4,4,5,5- tetramethyl-1,3,2- dioxaborolan-2-yl)- 1H-pyrazole | ¹H NMR (400 MHz, DMSO-d₆) δ = 8.95 (d, J = 7.5 Hz, 1H), 8.41 (s, 1H), 8.23 (d, J = 8.0 Hz, 1H), 8.09 (s, 1H), 7.97 (d, J = 8.5 Hz, 1H), 7.86 (s, 1H), 7.73 (t, J = 7.5 Hz, 1H), 7.57 (t, J = 7.3 Hz, 1H), 7.38-7.30 (m, 2H), 7.28-7.21 (m, 2H), 5.89 (t, J = 7.3 Hz, 1H), 3.94 (s, 3H), 2.28 (s, 3H), 1.61 (d, J = 7.0 Hz, 3H). LCMS (ESI): m/z = 371.3 [M + H]⁺. |
| | (R)-2-methyl-N-(1-(2-(1- | | |
| | methyl-1H-pyrazol-4- | | |
| | yl)quinolin-4-yl)ethyl) | | |
| | benzamide | | |
| 50 | 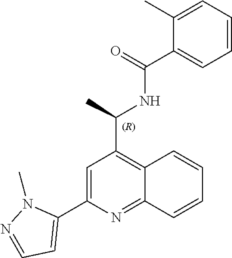 | 1-methyl-5-(4,4,5,5- tetramethyl-1,3,2- dioxaborolan-2-yl)- 1H-pyrazole | ¹H NMR (400 MHz, MeOD-d₄) δ = 8.34 (d, J = 8.4 Hz, 1H), 8.15 (d, J = 8.8 Hz, 1H), 7.90 (s, 1H), 7.85-7.79 (m, 1H), 7.70 (dt, J = 1.3, 7.7 Hz, 1H), 7.57 (d, J = 2.2 Hz, 1H), 7.38-7.31 (m, 2H), 7.28-7.22 (m, 2H), 6.85 (d, J = 2.2 Hz, 1H), 6.05 (d, J = 7.0 Hz, 1H), 4.34 (s, 3H), 2.35 (s, 3H), 1.72 (d, J = 7.0 Hz, 3H). LCMS (ESI): m/z = 371.3 [M + H]⁺. |
| | (R)-2-methyl-N-(1-(2-(1- | | |
| | methyl-1H-pyrazol-5- | | |
| | yl)quinolin-4- | | |
| | yl)ethyl)benzamide | | |
| 58 | 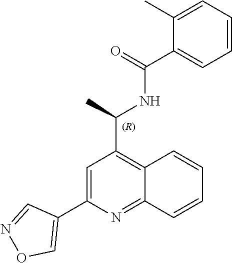 | 4-(4,4,5,5- tetramethyl-1,3,2- dioxaborolan-2- yl)isoxazole | ¹H NMR (400 MHz, MeOD-d₄) δ = 9.27 (s, 1H), 8.29 (d, J = 8.0 Hz, 1H), 7.86 (br d, J = 2.5 Hz, 2H), 7.67 (br s, 1H), 7.49-7.44 (m, 2H), 7.39-7.34 (m, 1H), 7.28 (br d, J = 3.0 Hz, 2H), 5.88 (d, J = 7.0 Hz, 1H), 2.40 (s, 3H), 1.68 (d, J = 7.5 Hz, 3H). LCMS (ESI): m/z = 358.1 [M + H]⁺. |
| | (R)-N-(1-(2-(isoxazol-4- | | |
| | yl)quinolin-4-yl)ethyl)-2- | | |
| | methylbenzamide | | |

### Example 51: (2R)—N-(3-((1-(3-(1H-pyrazol-4-yl)naphthalen-1-yl)ethyl)carbamoyl)-4-methylphenyl)piperidine-2-carboxamide

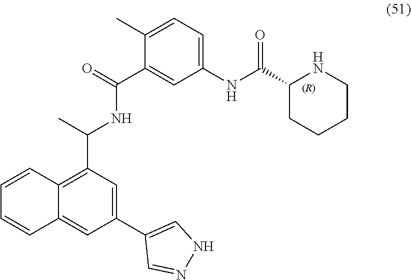

Step 1. Preparation of tert-butyl (1-(3-(1-(tetrahydro-2H-pyran-2-yl)-1H-pyrazol-4-yl) naphthalen-1-yl)ethyl)carbamate

To a solution of tert-butyl(1-(3-bromonaphthalen-1-yl)ethyl)carbamate (500.0 mg, 1.43 mmol) in 1,4-dioxane (15.0 mL) and H₂O (3.0 mL) were added Na₂CO₃ (454 mg, 4.28 mmol), 1-(tetrahydro-2H-pyran-2-yl)-4-(4,4,5,5-tetramethyl-1,3,2-dioxaborolan-2-yl)-1H-pyrazole (794 mg, 2.86 mmol), and Pd(dppf)Cl₂ (104 mg, 0.143 mmol) at 30° C. The mixture was bubbled with N₂ for 1 min and stirred at 100° C. for 16 h. LCMS showed that a desired product was formed. The reaction mixture was evaporated in vacua and partitioned between ethyl acetate/water (50 mL/50 mL). The organic layer was separated, dried with anhydrous Na₂SO₄, filtered, evaporated in vacuo, and purified with silica chromatography using PE:EtOAc=0%-30% to give tert-butyl(1-(3-(1-(tetrahydro-2H-pyran-2-yl)-1H-pyrazol-4-yl)naphthalen-1-yl)ethyl)carbamate (690 mg, 115%) as a colorless oil. ¹H NMR (400 MHz, MeOD-d₄) δ=831 (s, 1H), 8.15-8.09 (m, 1H), 8.05 (s, 1H), 7.98 (s, 1H), 7.94-7.87 (m, 1H), 7.79 (s, 1H), 7.53-7.46 (m, 2H), 7.42-7.33 (m, 1H), 5.48 (dd, J=2.1, 10.1 Hz, 2H), 4.61 (s, 2H), 4.16-4.05 (m, 2H), 387-3.74 (m, 1H), 2.10 (br d, J=10.1 Hz, 2H), 2.03 (s, 1H), 1.87-1.66 (m, 2H), 1.59 (br d, J=6.9 Hz, 3H), 1.46 (br s, 7H), 1.34 (s, 2H), 1.28-1.25 (m, 2H), 1.22 (s, 11H). LCMS (ESI): m/z=282.0, [M-THP-tBu]⁺.

### Step 2. Preparation of 1(3-(1H-pyrazol-4-yl)naphthalen-1-yl)ethan-1-amine

To a solution of tert-butyl(1-(3-(1-(tetrahydro-2H-pyran-2-yl)-1H-pyrazol-4-yl)naphthalen-1-yl)ethyl)carbamate (690 mg, 1.64 mmol) in dichloromethane (10.0 mL) was added 4M HCl/dioxane (730 mg, 20 mmol) at 25° C. The reaction mixture was stirred at 25° C. for 3 h. LCMS showed that the starting material was consumed and the desired product was formed. The reaction mixture was concentrated in vacuo to give a crude, which was dissolved in H₂O (10 mL), then basified with NH₃·H₂O to pH=8˜9. The mixture was then extracted with ethyl acetate (10 mL×3). The combined organic layer was dried with Na₂SO₄, filtered, concentrated in vacuo to give 1-(3-(1H-pyrazol-4-yl)naphthalen-1-yl)ethan-1-amine (300 mg, 77.2%) as a brown gum. LCMS (ESI): m/z=221.0 [M−NH₃+H]⁺; 239.0 [M−NH₃+H₂O+H]⁺.

### Step 3. Preparation of t-butyl(2R)-2-(3-((1-(3-(1H-pyrazol-4-yl)naphthalen-1-yl)ethyl)carbamoyl)-4-methylphenyl)carbamoyl)piperidine-1-carboxylate

Using a procedure analogous to Example 4, Step 1 with 1-(3-(1H-pyrazol-4-yl)naphthalen-1-yl)ethan-1-amine (183 mg, 0.506 mmol) and (R)-5-(1-(tert-butoxycarbonyl)piperidine-2-carboxamido)-2-methylbenzoic acid (120.0 mg, 0.506 mmol) as the reactants affords t-butyl(2R)-2-((3-((1-(3-(1H-pyrazol-4-yl)naphthalen-1-yl)ethyl)carbamoyl)-4-methylphenyl)carbamoyl)piperidine-1-carboxylate (180 mg, 61.2%) as a colorless oil. LCMS (ESI): m/z=582.3 [M+H]⁺.

### Step 4. Preparation of (2R)—N-(3-((1-(3-(1H-pyrazol-4-yl)naphthalen-1-yl)ethyl)carbamoyl)-4-methylphenyl)piperidine-2-carboxamide

Using a procedure analogous to Example 4 Step 2 with t-butyl(2R)-2-((3-((1-(3-(1H-pyrazol-4-yl)naphthalen-1-yl)ethyl)carbamoyl)-4-methylphenyl)carbamoyl)piperidine-1-carboxylate (20.0 mg, 0.034 mmol) as the reactant affords (2R)—N-(3-((1-(3-(1H-pyrazol-4-yl)naphthalen-1-yl)ethyl)carbamoyl)-4-methylphenyl)piperidine-2-carboxamide (5.38 mg, 32%) as a white solid. ¹H NMR (400 MHz, DMSO-dc) δ=1328-12.91 (m, 1H), 9.77-9.66 (m, 1H), 8.90 (br d, J=8.1 Hz, 1H), 8.22-8.14 (m, 1H), 8.05 (s, 1H), 7.95-7.87 (m, 2H), 7.72 (s, 1H), 7.57-7.48 (m, 2H), 7.14 (d, J=8.4 Hz, 1H), 5.97-5.87 (m, 1H), 3.36 (br s, 4H), 2.20 (s, 2H), 1.82-1.73 (m, 2H), 1.59 (br d, J=6.8 Hz, 2H), 1.53-1.33 (m, 3H). LCMS (ESI): m/z=482.4 [M+H]⁺.

### Example 52: (R)—N-(3-(((R*)-1-(3-(1H-pyrazol-4-yl)naphthalen-1-yl)ethyl)carbamoyl)-4-methylphenyl)piperidine-2-carboxamide, ENT-1 (52) and (R)—N-(3-(((R*)-1-(3-(H-pyrazol-4-yl)naphthalen-1-yl)ethyl)carbamoyl)-4-methylphenyl)piperidine-2-carboxamide, ENT-2 (53)

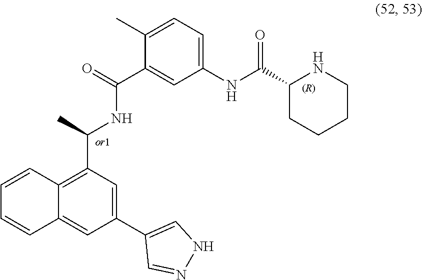

(R)—N-(3-(((R*)-1-(3-(1H-pyrazol-4-yl)naphthalen-1-yl)ethyl)carbamoyl)-4-methylphenyl) piperidine-2-carboxamide, ENT-1 and (R)—N-(3-(((R*)-1-(3-(1H-pyrazol-4-yl)naphthalen-1-yl)ethyl)carbamoyl)-4-methylphenyl)piperidine-2-carboxamide, ENT-2 were obtained by SFC separation in a similar manner to Examples 8-9.

### Example 54: N-(3-methyl-4-((1-(3-(1-methyl-1H-pyrazol-4-yl)naphthalen-1-yl)ethyl)carbamoyl)benzyl)thiazole-4-carboxamide

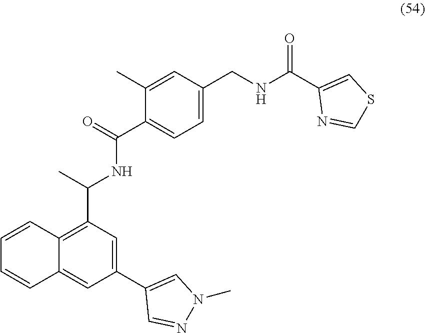 [SMILES: Cc1cc(CNC(=O)c2cscn2)ccc1C(=O)NC(C)c1cc(-c2cnn(C)c2)cc2ccccc12]

Using a procedure analogous to Example 4 Step 1 with 4-(aminomethyl)-2-methyl-N-(1-(3-(1-methyl-1H-pyrazol-4-yl)naphthalen-1-yl)ethyl)benzamide (50.0 mg, 0.13 mmol, Example 5) and thiazole-4-carboxylic acid (16.2 mg, 0.125 mmol) as the reactants affords N-(3-methyl-4-((1-(3-(1-methyl-1H-pyrazol-4-yl)naphthalen-1-yl)ethyl)carbamoyl)benzyl)thiazole-4-carboxamide (16.68, 26%) as a white solid after lyophilization. ¹H NMR (400 MHz, DMSO-d5) δ=9.19 (d, J=2.0 Hz, 1H), 9.06 (t, J=6.4 Hz, 1H), 8.82 (d, J=8.1 Hz, 1H), 8.32 (d, J=2.0 Hz, 1H), 8.20 (s, 1H), 8.18-8.15 (m, 1H), 7.99 (s, 1H), 7.94 (s, 1H), 7.89 (dd, J=2.9, 6.8 Hz, 1H), 7.83 (d, J=1.1 Hz, 1H), 7.53-7.48 (m, 2H), 7.29-7.25 (m, 1H), 7.19-7.16 (m, 2H), 5.91 (br t, J=7.5 Hz, 1H), 4.43 (d, J=6.2 Hz, 2H), 3.90 (s, 3H), 2.26 (s, 3H), 1.59 (d, J=7.0 Hz, 3H). LCMS (ESI): m/z=510.3 [M+H]⁺.

### Example 56: 5-{4-[(1R)-1-{[5-(aminomethyl)-2-methylphenyl]formamido}ethyl]quinolin-2-yl}-N,N-dimethyl-1H-pyrrole-3-carboxamide

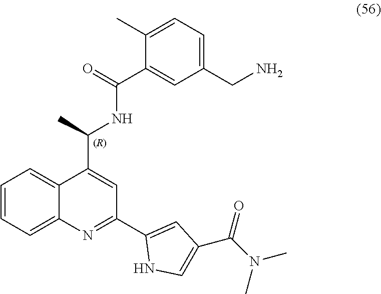

### Step 1. Preparation of tert-butyl(R)-(3-((1-(2-(4-(dimethylcarbamoyl)-1H-pyrrol-2-yl)quinolin-4-yl)ethyl)carbamoyl)-4-methylbenzyl)carbamate

Using a procedure analogous to Example 21 Step 1 with (R)-5-(4-(1-(5-(((tert-butoxycarbonyl)amino)methyl)-2-methylbenzamido)ethyl)quinolin-2-yl)-1H-pyrrole-3-carboxylic acid (150.0 mg, 0.284 mmol, prepared in Example 20 Step 1) and Me₂NH (231 mg, 2.84 mmol) as the reactants affords tert-butyl(R)-(3-((1-(2-(4-(dimethylcarbamoyl)-1H-pyrrol-2-yl)quinolin-4-yl)ethyl) carbamoyl)-4-methylbenzyl)carbamate (100 mg, 63.4%) as a yellow oil, which was used in the next step directly. LCMS (ESI): m/z=500.3 [M+H−tBu]⁺.

### Step 2. Preparation of (R)-5-(4-(1-(5-(aminomethyl)-2-methylbenzamido)ethyl)quinolin-2-yl)-N,N-dimethyl-1H-pyrrole-3-carboxamide

Using a procedure analogous to Example 21 Step 2 with tert-butyl(R)-(3-((1-(2-(4-(dimethylcarbamoyl)-1H-pyrrol-2-yl)quinolin-4-yl)ethyl)carbamoyl)-4-methylbenzyl)carbamate (100 mg, 0.180 mmol) as the reactant affords (R)-5-(4-(1-(5-(aminomethyl)-2-methylbenzamido)ethyl)quinolin-2-yl)-N,N-dimethyl-1H-pyrrole-3-carboxamide (9 mg, 11.6%) as a white solid. ¹H NMR (400 MHz, MeOD-d₄) δ=8.26 (d, J=8.3 Hz, 1H), 8.09 (d, J=3.1 Hz, 1H), 7.95 (s, 1H), 7.77 (t, J=7.3 Hz, 1H), 7.61 (t, J=7.2 Hz, 1H), 7.44 (d, J=1.5 Hz, 1H), 7.42-7.35 (m, 2H), 7.27 (d, J=7.8 Hz, 1H), 7.22 (d, J=1.5 Hz, 1H), 6.05 (q, J=6.9 Hz, 1H), 3.88 (s, 2H), 3.33 (br d, J=1.5 Hz, 3H), 3.15 (br s, 4H), 2.37 (s, 3H), 1.75 (d, J=7.0 Hz, 3H). LCMS (ESI): m/z=456.3 [M+H]⁺.

### Example 61: (R)-2-methyl-N-(1-(2-(1-methyl-1H-pyrazol-4-yl)quinolin-4-yl)ethyl)-5-(methylsulfonamidoethyl)benzamide

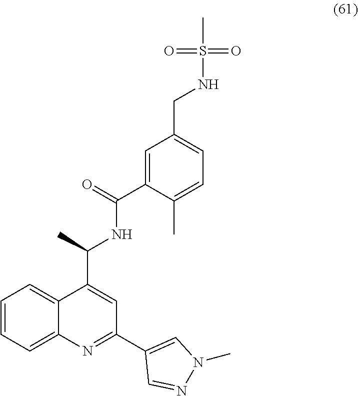 [SMILES: Cc1ccc(CNS(C)(=O)=O)cc1C(=O)N[C@H](C)c1cc(-c2cnn(C)c2)nc2ccccc12]

To a solution of (R)-5-(aminomethyl)-2-methyl-N-(1-(2-(1-methyl-1H-pyrazol-4-yl)quinolin-4-yl)ethyl)benzamide (Example 11) (158.0 mg, 0.396 mmol) in THF (15 mL) at 0° C. were added TEA (120 mg, 1.19 mmol) and Ms₂O (103 mg, 0.593 mmol). The reaction mixture was stirred at 20° C. for 15 h. LCMS showed a mass peak of the desired product. The reaction mixture was partitioned between ethyl acetate and H₂O (50/50 mL). The organic layer was separated, and the aqueous layer was re-extracted with ethyl acetate (50 mL). The combined organic layer was washed with brine, dried with anhydrous Na₂SO₄, and evaporated in vacuo to afford a crude. The crude was dissolved in 5 mL DMF then purified by prep-HPLC to afford (R)-2-methyl-N-(1-(2-(1-methyl-1H-pyrazol-4-yl)quinolin-4-yl)ethyl)-5-(methylsulfonamidomethyl)benzamide (26 mg, 14%) as a yellow solid after solvent removal and lyophilization. ¹H NMR of PLASMA-648: 1H NMR (400 MHz, MeOD) 5 ppm 8.43-3.26 (m, 2H), 3.22 (s, 1H), 8.16-8.02 (m, 1H), 7.88 (s, 1H), 7.80 (t, J=7.7 Hz, 1H), 7.65 (t, J=7.1 Hz, 1H), 7.45 (s, 1H), 7.37 (d, J=7.3 Hz, 1H), 7.27 (d, J=7.8 Hz, 1H), 6.05 (q, J=6.9 Hz, 1H), 4.27 (s, 2H), 4.03 (s, 3H), 2.90 (s, 3H), 2.36 (s, 3H), 1.75 (d, J=7.0 Hz, 3H). LCMS (ESI): m/z=478.3 [M+H]⁺.

### Prep-HPLC Condition:

Column: Boston Prime C18 150*30 mm*5 μm;
Mobile phase: water (FA v/v)-ACN; B %: 0%-35%,
Flow Rate (mL/min): 30 mL/min.

### Example 62: N-(3-methyl-4-((1-(3-(1-methyl-1H-pyrazol-4-yl)naphthalen-1-yl)ethyl)carbamoyl)benzyl)-1H-imidazole-2-carboxamide

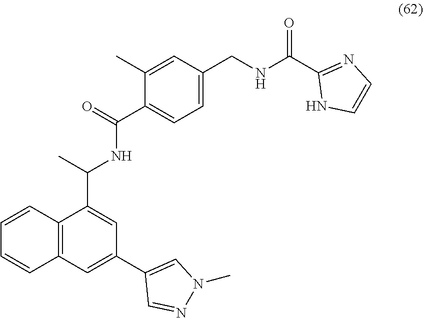 [SMILES: Cc1cc(CNC(=O)c2ncc[nH]2)ccc1C(=O)NC(C)c1cc(-c2cnn(C)c2)cc2ccccc12]

To a mixture of 4-(aminomethyl)-2-methyl-N-(1-(3-(1-methyl-1H-pyrazol-4-yl)naphthalen-1-yl)ethyl)benzamide (Example 5) (40.0 mg, 0.10 mmol), DIEA (64.9 mg, 0.502 mmol), and 1H-imidazole-2-carboxylic acid (11.3 mg, 0.100 mmol) in DMF (0.502 mL) was added PyBOP (104 mg, 0.201 mmol). The reaction mixture was stirred at 25° C. for 12 h. LCMS showed the desired product was formed. H₂O (5 mL) was then added to the reaction, and the mixture was extracted with ethyl acetate (2 mL×2). The organic extract was concentrated in vacuo and purified by prep-HPLC to afford N-(3-methyl-4-((1-(3-(1-methyl-1H-pyrazol-4-yl)naphthalen-1-yl)ethyl)carbamoyl)benzyl)-1H-imidazole-2-carboxamide (22.13 mg, 45%) as a white solid after lyophilization. ¹H NMR (400 MHz, DMSO-de) δ=12.99 (br s, 1H), 8.98 (t, J=6.4 Hz, 1H), 8.82 (d, J=8.1 Hz, 1H), 8.22-8.14 (m, 2H), 7.99 (s, 1H), 7.94 (s, 1H), 7.89 (br dd, J=2.8, 6.7 Hz, 1H), 7.83 (d, J=1.2 Hz, 1H), 7.53-7.48 (m, 2H), 7.29-7.25 (m, 2H), 7.19-7.14 (m, 2H), 7.04 (s, 1H), 5.91 (br t, J=7.3 Hz, 1H), 4.40 (d, J=6.4 Hz, 2H), 3.90 (s, 3H), 2.26 (s, 3H), 1.59 (d, J=6.8 Hz, 3H). LCMS (ESI): m/z=4934 [M+H]⁺.

### Prep-HPLC Condition:

Column: Boston Prime C18 150*30 mm*5 μm;
Mobile phase: water (ammonia hydroxide v/v)-ACN; B %: 35%-55%,
Flow Rate (mL/min): 35 mL/min.

### Example 63: methyl(R,E)-4-(2-(3-(2-((1-(2-(1-methyl-1H-pyrazol-4-yl)quinolin-4-yl)ethyl)carbamoyl)phenyl)propanoyl)hydrazineyl)-4-oxobut-2-enoate

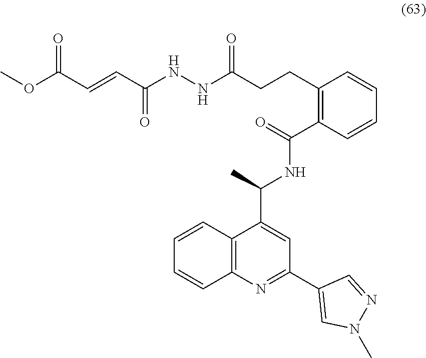 [SMILES: COC(=O)/C=C/C(=O)NNC(=O)CCc1ccccc1C(=O)N[C@H](C)c1cc(-c2cnn(C)c2)nc2ccccc12]

### Step 1. Preparation of 2-(3-methoxy-3-oxopropyl)benzoic acid

To a solution of 2-(2-carboxyethyl)benzoic acid (1 g, 1.54 mmol) in methanol (15 mL) at 25° C. was added H₂SO₄ (0.2 mL). The reaction was then stirred at 25° C. for 3 h. TLC showed that the starting material was consumed and one new spot was formed. The reaction was concentrated in vacuo then added aq. sat. NaHCO₃ (10 mL) and DCM (10 mL). The aqueous was adjusted to pH-3 with 1N HCl, then extracted with DCM (10 mL×2). The combined organic extract was dried using Na₂SO₄, filtered, and concentrated in vacuo to give 2-(3-methoxy-3-oxopropyl)benzoic acid (800 mg, 70%) as a white solid. ¹H NMR (400 MHz, CDCl₃) δ=806 (dd, J=1.3, 8.1 Hz, 1H), 7.49 (dd, J=1.4, 7.6 Hz, 1H), 7.36-7.30 (m, 2H), 3.67 (s, 3H), 3.34 (t, J=7.6 Hz, 2H), 2.72 (t, J=7.6 Hz, 2H). LCMS (ESI): m/z=209.2, [M+H]⁺.

### Step 2. Preparation of methyl(R)-3-(2-((1-(2-(1-methyl-1H-pyrazol-4-yl)quinolin-4-yl)ethyl)carbamoyl)phenyl)propanoate

To a solution of 2-(3-methoxy-3-oxopropyl)benzoic acid (165 mg, 0.793 mmol) in DMF (7.93 mL) at 20° C. were added DIEA (307 mg, 2.38 mmol), HATU (452 mg, 1.19 mmol), and then (R)-1-(2-(1-methyl-1H-pyrazol-4-yl)quinolin-4-yl)ethan-1-amine (200.0 mg, 0.793 mmol). The reaction mixture was stirred at 20° C. for 15 h. LCMS showed that the main peak was of the desired product. H₂O (20 mL) was then added to the reaction, and the mixture was extracted with ethyl acetate (10 mL×2). The organic layers were combined, dried over Na₂SO₄, filtered, concentrated in vacuo, and purified with silica gel column chromatography (PE:EA=0%-50%) to give methyl(R)-3-(2-((1-(2-(1-methyl-1H-pyrazol-4-yl)quinolin-4-yl)ethyl)carbamoyl)phenyl) propanoate (330 mg, 94.1%) as a colorless oil. LCMS (ESI): m/z=443.3 [M+H]⁺.

### Step 3. Preparation of methyl (R)-2-(3-hydrazineyl-3-oxopropyl)-N-(1-(2-(1-methyl-1H-pyrazol-4-yl)quinolin-4-yl)ethyl)benzamide

To a solution of methyl(R)-3-(2-((1-(2-(1-methyl-1H-pyrazol-4-yl)quinolin-4-yl)ethyl)carbamoyl)phenyl)propanoate (330 mg, 0.75 mmol) in EtOH (7.46 mL) was added NH₂NH₂·H₂O (700 mg, 12 mmol). The reaction mixture was stirred at 80° C. for 16 h. LCMS showed a major product was formed. The reaction was cooled to 20° C. and concentrated under reduced pressure. The residue was azeotroped with toluene (5 mL×3) to give (R)-2-(3-hydrazineyl-3-oxopropyl)-N-(1-(2-(1-methyl-1H-pyrazol-4-yl)quinolin-4-yl)ethyl)benzamide (330 mg, 100%) as a yellow solid, which was used for the next step directly. LCMS (ESI): m/z=443.2 [M+H]⁺.

### Step 4. Preparation of methyl(R,E)-4-(2-(3-(2-((1-(2-(1-methyl-1H-pyrazol-4-yl)quinolin-4-yl)ethyl)carbamoyl)phenyl)propanoyl)hydrazineyl)-4-oxobut-2-enoate

To a mixture of (E)-4-methoxy-4-oxobut-2-enoic acid (35.3 mg, 0.271 mmol) in DMF (1.36 mL) were added DIEA (105 mg, 0.814 mmol) and HATU (124 mg, 0.325 mmol). The mixture was stirred at 20° C. for 1 h before adding (R)-2-(3-hydrazineyl-3-oxopropyl)-N-(1-(2-(1-methyl-1H-pyrazol-4-yl)quinolin-4-yl)ethyl)benzamide (120.0 mg, 0.271 mmol). The reaction was then stirred at 20° C. for 16 h. LCMS showed the desired product was formed. H₂O (5 mL) was then added to the reaction, and the mixture was extracted with ethyl acetate (10 mL×2). The organic layers was combined, dried over Na₂SO₄, filtered, concentrated in vacuo, and purified by prep-HPLC to afford methyl(R,E)-4-(2-(3-(2-((1-(2-(1-methyl-1H-pyrazol-4-yl)quinolin-4-yl)ethyl)carbamoyl)phenyl) propanoyl)hydrazineyl)-4-oxobut-2-enoate (19.65 mg, 13.1%) as a white solid after lyophilization. ¹H NMR (400 MHz, DMSO-d₆) δ=10.54 (br s, 1H), 10.19 (br s, 1H), 9.02 (d, J=7.9 Hz, 1H), 8.43 (s, 1H), 8.23 (d, J=8.3 Hz, 1H), 8.11 (s, 1H), 7.99-7.94 (m, 1H), 7.86 (s, 1H), 7.76-7.70 (m, 1H), 7.61-7.55 (m, 1H), 7.40-7.25 (m, 4H), 7.06 (d, J=15.6 Hz, 1H), 6.68 (d, J=15.6 Hz, 1H), 5.90 (br t, J=7.3 Hz, 1H), 3.93 (s, 3H), 3.74 (s, 3H), 2.98-2.90 (m, 2H), 1.63 (d, J=6.9 Hz, 3H). LCMS (ESI): m/z=555.2 [M+H]⁺.

### Prep-HPLC Condition:

Column: Boston Prime C18 150*30 mm*5 μm;
Mobile phase: water (ammonia hydroxide v/v)-ACN; B %: 18%-38%.
Flow Rate (mL/min): 35 mL/min.

### Example 64: methyl (E)-4-(2-(3-(2-((1-(3-(1-methyl-1H-pyrazol-4-yl)naphthalen-1-yl)ethyl)carbamoyl)phenyl)propanoyl)hydrazineyl)-4-oxobut-2-enoate

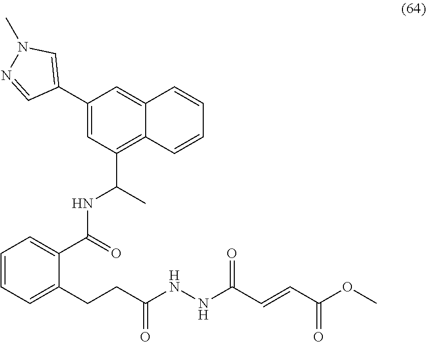 [SMILES: COC(=O)/C=C/C(=O)NNC(=O)CCc1ccccc1C(=O)NC(C)c1cc(-c2cnn(C)c2)cc2ccccc12]

### Step 1. Preparation of methyl 3-(2-((1-(3-(1-methyl-1H-pyrazol-4-yl)naphthalen-1-yl)ethyl)carbamoyl)phenyl)propanoate

Using a procedure analogous to Example 63 Step 2 with 2-(3-methoxy-3-oxopropyl)benzoic acid (100 mg, 0.480 mmol) and 1-(3-(1-methyl-1H-pyrazol-4-yl)naphthalen-1-yl)ethan-1-amine (P5) (121 mg, 0.480 mmol) as the reactants affords methyl 3-(2-((1-(3-(1-methyl-1H-pyrazol-4-yl)naphthalen-1-yl)ethyl)carbamoyl)phenyl)propanoate (200 mg, 94.3%) as a colorless oil. LCMS (ESI): m/z=442.2, [M+H]⁺.

### Step 2. Preparation of 2-(3-hydrazineyl-3-oxopropyl)-N-(1-(3-(1-methyl-1H-pyrazol-4-yl)naphthalen-1-yl)ethyl)benzamide

Using a procedure analogous to Example 63 Step 3 with methyl 3-(2-((1-(3-(1-methyl-1H-pyrazol-4-yl)naphthalen-1-yl)ethyl)carbamoyl)phenyl)propanoate (180 mg, 0.408 mmol) as the reactant affords 2-(3-hydrazineyl-3-oxopropyl)-N-(1-(3-(1-methyl-1H-pyrazol-4-yl)naphthalen-1-yl)ethyl)benzamide (180 mg, 100%) as a colorless oil. LCMS (ESI): m/z=442.3, [M+H]⁺.

### Step 3. Preparation of methyl (E)-4-(2-(3-(2-((1-(3-(1-methyl-1H-pyrazol-4-yl)naphthalen-1-yl)ethyl)carbamoyl)phenyl)propanoyl)hydrazineyl)-4-oxobut-2-enoate

Using a procedure analogous to Example 63 Step 3 with 2-(3-hydrazineyl-3-oxopropyl)-N-(1-(3-(1-methyl-1H-pyrazol-4-yl)naphthalen-1-yl)ethyl)benzamide (47.1 mg, 0.362 mmol) and (E)-4-methoxy-4-oxobut-2-enoic acid (160.0 mg, 0.362 mmol) as the reactants affords methyl (E)-4-(2-(3-(2-((1-(3-(1-methyl-1H-pyrazol-4-yl)naphthalen-1-yl)ethyl)carbamoyl)phenyl)propanoyl)hydrazineyl)-4-oxobut-2-enoate (3.1 mg, 1.55%) as a white solid. ¹H NMR (400 MHz, DMSO-d₆) δ=10.57 (br s, 1H), 10.20 (s, 1H), 8.95 (br d, J=8.1 Hz, 1H), 8.22 (s, 1H), 8.20-8.16 (m, 1H), 8.00 (s, 1H), 7.96 (s, 1H), 7.92-7.88 (m, 1H), 7.85 (s, 1H), 7.56-7.49 (m, 2H), 7.39-7.23 (m, 4H), 7.07 (d, J=15.5 Hz, 1H), 6.68 (d, J=15.7 Hz, 1H), 5.94 (br t, J=7.2 Hz, 1H), 3.90 (s, 3H), 3.74 (s, 3H), 2.95 (br t, J=7.8 Hz, 2H), 1.63 (br d, J=6.8 Hz, 3H). LCMS (ESI): m/z=554.4, [M+H]⁺.

### Example 66: (R)—N-(4-methyl-3-(((R)-1-(3-(1-methyl-1H-pyrazol-4-yl)naphthalen-1-yl)ethyl)carbamoyl)phenyl)piperidine-2-carboxamide, ENT-1

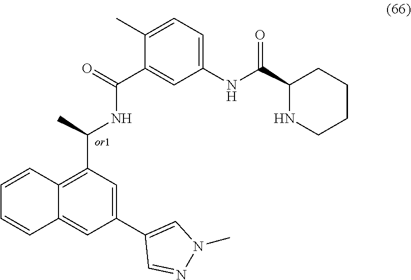 [SMILES: Cc1ccc(NC(=O)[C@H]2CCCCN2)cc1C(=O)N[C@H](C)c1cc(-c2cnn(C)c2)cc2ccccc12]

### Step 1. Preparation of tert-butyl(R)-2-((4-methyl-3-(((R*)-1-(3-(1-methyl-1H-pyrazol-4-yl)naphthalen-1-yl)ethyl)carbamoyl)phenyl)carbamoyl)piperidine-1-carboxylate, ENT-1

tert-butyl(2R)-2-((4-methyl-3-((1-(3-(1-methyl-1H-pyrazol-4-yl)naphthalen-1-yl)ethyl)carbamoyl)phenyl)carbamoyl)piperidine-1-carboxylate (prepared in Example 4, Step 1) (222 mg, 0.373 mmol) was separated by SFC. The fractions were evaporated under vacuo and dried by lyophilization to afford tert-butyl(R)-2-((4-methyl-3-(((R)-1-(3-(1-methyl-1H-pyrazol-4-yl) naphthalen-1-yl)ethyl)carbamoyl)phenyl)carbamoyl)piperidine-1-carboxylate, ENT-1 (91 mg, first peak) and tert-butyl(R)-2-((4-methyl-3-(((R)-1-(3-(1-methyl-1H-pyrazol-4-yl)naphthalen-1-yl)ethyl)carbamoyl)phenyl)carbamoyl)piperidine-1-carboxylate, ENT-2 (95.7 mg, second peak) as colourless gums. LCMS (ESI): m/z=596.4, [M+H]⁺ and LCMS (ESI): m/z=596.1, [M+H]⁺, respectively.

### SFC Condition:

Column: DAICEL CHIRALCEL OD-H (250 mm*30 mm, 5 μm);
Mobile phase: CO₂-EtOH (0.1% NH₃·H₂O); B %: 30%-30%,
Flow Rate (mL/min): 80 mL/min.

### Step 2. Preparation of (R)—N-(4-methyl-3-(((R)-1-(3-(1-methyl-1H-pyrazol-4-yl)naphthalen-1-yl)ethyl)carbamoyl)phenyl)piperidine-2-carboxamide, ENT-1

tert-butyl(R)-2-((4-methyl-3-(((R*)-1-(3-(1-methyl-1H-pyrazol-4-yl)naphthalen-1-yl)ethyl) carbamoyl)phenyl)carbamoyl)piperidine-1-carboxylate, ENT-1 (91 mg 0.15 mmol) was deprotected using a procedure analogous to Example 4, Step 2 to afford (R)—N-(4-methyl-3-(((Rn-1-(3-(1-methyl-1H-pyrazol-4-yl)naphthalen-1-yl)ethyl)carbamoyl)phenyl)piperidine-2-carboxamide, ENT-1 (6.01 mg, 9%) as a white solid. ¹H NMR (400 MHz, CD3SOCD3, 295 K) δ (ppm)=9.78-9.66 (m, 1H), 8.90 (d, J=7.9 Hz, 1H), 8.26 (s, 2H), 8.21-8.16 (m, 2H), 8.04-7.97 (m, 3H), 7.94-7.88 (m, 1H), 7.85 (s, 1H), 7.79 (d, J=2.4 Hz, 1H), 7.56-7.50 (m, 4H), 7.15 (d, J=8.6 Hz, 1H), 5.98-5.86 (m, 1H), 3.93 (s, 4H), 2.70-2.66 (m, 3H), 2.33 (s, 1H), 2.22 (s, 4H), 1.84-1.75 (m, 3H), 1.60 (d, J=6.9 Hz, 4H), 1.46-1.39 (m, 3H). LCMS (ESI): m/z=496.4, [M+H]⁺

### Example 67: (R)—N-(4-methyl-3-(((R)-1-(3-(1-methyl-1H-pyrazol-4-yl)naphthalen-1-yl)ethyl)carbamoyl)phenyl)piperidine-2-carboxamide, ENT-2

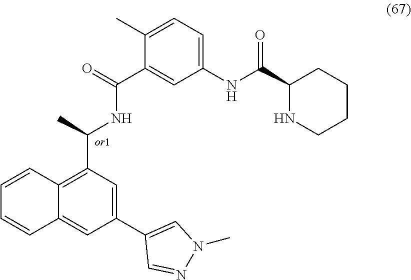 [SMILES: Cc1ccc(NC(=O)[C@H]2CCCCN2)cc1C(=O)N[C@H](C)c1cc(-c2cnn(C)c2)cc2ccccc12]

Tert-butyl(R)-2-((4-methyl-3-(((R)-1-(3-(1-methyl-1H-pyrazol-4-yl)naphthalen-1-yl)ethyl) carbamoyl)phenyl)carbamoyl)piperidine-1-carboxylate, ENT-2 was deprotected using a procedure analogous to Example 4 Step 2 to afford (R)—N-(4-methyl-3-(((R)-1-(3-(1-methyl-1H-pyrazol-4-yl)naphthalen-1-yl)ethyl)carbamoyl)phenyl)piperidine-2-carboxamide, ENT-2 (12.8 mg, 17%) as a white solid. ¹H NMR (400 MHz, CD3SOCD3, 295 K) δ (ppm)=9.66 (s, 1H), 8.91 (d, J=7.9 Hz, 1H), 8.27 (s, 1H), 8.21-8.16 (m, 1H), 8.02 (s, 1H), 7.98 (s, 1H), 7.93-7.89 (m, 1H), 7.85 (s, 1H), 7.81 (s, 1H), 7.56-7.51 (m, 4H), 7.15 (d, J=8.4 Hz, 1H), 5.96-5.90 (m, 1H), 3.93 (s, 3H), 3.23 (dd, J=3.0, 9.8 Hz, 1H), 3.00-2.95 (m, 1H), 2.68 (br s, 1H), 2.45 (br s, 1H), 2.33 (br s, 1H), 2.22 (s, 3H), 1.83-1.76 (m, 2H), 1.59 (d, J=6.6 Hz, 3H), 1.52-1.39 (m, 4H). LCMS (ESI): m/z=496.4, [M+H]⁺.

### Example 68: (R)—N-(3-(((R)-1-(3-(1H-pyrazol-5-yl)naphthalen-1-yl)ethyl)carbamoyl)-4-methylphenyl)piperidine-2-carboxamide, ENT-1

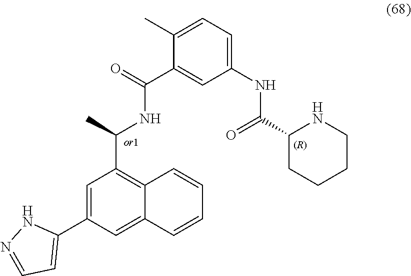

### Step 1. Preparation of (R)—N-(3-(((R*)-1-(3-(1H-pyrazol-5-yl)naphthalen-1-yl)ethyl)carbamoyl)-4-methylphenyl)piperidine-2-carboxamide, ENT-1

Tert-butyl(R)-2-((3-(((RS)-1-(3-(1H-pyrazol-5-yl)naphthalen-1-yl)ethyl)carbamoyl)-4-methylphenyl)carbamoyl)piperidine-1-carboxylate (Prepared in Example 65, Step 1) (200.0 mg, 0.344 mmol) was separated by Prep-SFC to afford tert-butyl(R)-2-((3-(((R)-1-(3-(1H-pyrazol-5-yl)naphthalen-1-yl)ethyl)carbamoyl)-4-methylphenyl)carbamoyl)piperidine-1-carboxylate, ENT-1 (50 mg, peak 1) as a colourless oil and tert-butyl(R)-2-((3-(((R)-1-(3-(1H-pyrazol-5-yl)naphthalen-1-yl)ethyl)carbamoyl)-4-methylphenyl)carbamoyl)piperidine-1-carboxylate, ENT-2 (38 mg, peak 2) as a white solid. LCMS (ESI): m/z=582.3, [M+H]⁺ for both.

### Prep-SFC Method:

Mobile phase: CO₂-EtOH (0.1% ammonium hydroxide)
Column: DAICEL CHIRALPAK AD (250 mm*30 mm, 10 μm)
Flow rate: 80 mL/min
Monitor wavelength: 220&254 nm
Gradient: B %, 50% to 50%, 45 min

### Step 2. Preparation of (R)—N-(3-(((R*)-1-(3-(1H-pyrazol-5-yl)naphthalen-1-yl)ethyl)carbamoyl)-4-methylphenyl)piperidine-2-carboxamide, ENT-1

tert-butyl(R)-2-((3-(((R)-1-(3-(1H-pyrazol-5-yl)naphthalen-1-yl)ethyl)carbamoyl)-4-methylphenyl)carbamoyl)piperidine-1-carboxylate, ENT-1 (50 mg, 0.10 mmol) was deprotected using a procedure analogous to Example 4 Step 2 to afford (R)—N-(3-(((R)-1-(3-(1H-pyrazol-5-yl)naphthalen-1-yl)ethyl)carbamoyl)-4-methylphenyl)piperidine-2-carboxamide, ENT-1 (8.78 mg, 18%) as a white solid. ¹H NMR (400 MHz, CD3OD, 298 K) δ (ppm)=8.30-8.25 (m, 1H), 8.24-8.19 (m, 1H), 8.15-8.06 (m, 1H), 8.02-7.92 (m, 1H), 7.79-7.69 (m, 1H), 7.64-7.46 (m, 5H), 7.23-7.17 (m, 1H), 6.89-6.80 (m, 1H), 6.16-6.05 (m, 1H), 3.36 (d, J=2.8 Hz, 1H), 3.17-3.07 (m, 1H), 2.74-2.61 (m, 1H), 2.33-2.29 (m, 3H), 1.99-1.86 (m, 2H), 1.83-1.71 (m, 4H), 1.70-1.59 (m, 1H), 1.57-1.41 (m, 3H). LCMS (ESI): m/z=482.4, [M+H]⁺.

### Example 69: (R)—N-(3-(((R)-1-(3-(1H-pyrazol-5-yl)naphthalen-1-yl)ethyl)carbamoyl)-4-methylphenyl)piperidine-2-carboxamide, ENT-2

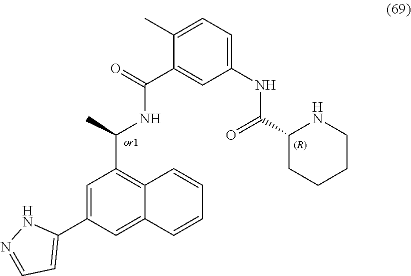

Tert-butyl(R)-2-((3-(((R)-1-(3-(1H-pyrazol-5-yl)naphthalen-1-yl)ethyl)carbamoyl)-4-methylphenyl)carbamoyl)piperidine-1-carboxylate, ENT-2 (prepared in Example 68 Step 1) (38.0 mg, 0.079 mmol) was deprotected using a procedure analogous to Example 4 Step 2 to afford (R)—N-(3-(((R*)-1-(3-(1H-pyrazol-5-yl)naphthalen-1-yl)ethyl)carbamoyl)-4-methylphenyl) piperidine-2-carboxamide, ENT-2 (15.83 mg, 40%) as a yellow solid. ¹H NMR (400 MHz, CD3OD, 298 K) δ (ppm)=10.20 (s, 1H), 8.37-8.24 (m, 2H), 8.12-8.00 (m, 3H), 7.70-7.58 (m, 3H), 7.56-7.45 (m, 1H), 7.23 (d, J=8.4 Hz, 1H), 7.10-7.02 (m, 1H), 6.17-6.02 (m, 1H), 3.99-3.81 (m, 1H), 3.46 (br d, J=12.6 Hz, 1H), 3.18-3.02 (m, 1H), 2.34-2.25 (m, 4H), 2.05-1.87 (m, 2H), 1.85-1.79 (m, 3H), 1.77-1.61 (m, 3H). LCMS (ESI): m/z=482.4, [M+H]⁺.

### Example 70: methyl 5-(4-(1-(5-(aminomethyl)-2-methylbenzamido)ethyl)naphthalen-2-yl)-1H-pyrrole-3-carboxylate

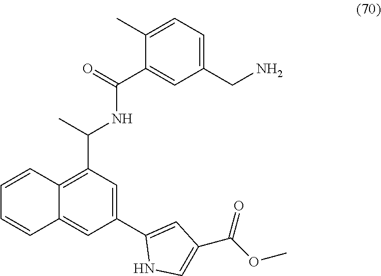 [SMILES: COC(=O)c1c[nH]c(-c2cc(C(C)NC(=O)c3cc(CN)ccc3C)c3ccccc3c2)c1]

### Step 1. Preparation of methyl 5-(4-(1-((tert-butylsulfinyl)amino)ethyl)naphthalen-2-yl)-1H-pyrrole-3-carboxylate

To a solution of N-(1-(3-bromonaphthalen-1-yl)ethyl)-2-methylpropane-2-sulfinamide (P12) (100.0 mg, 0.282 mmol) in 1,4-dioxane (3.0 mL) and H₂O (0.5 mL) were added methyl 5-(4,4,5,5-tetramethyl-1,3,2-dioxaborolan-2-yl)-1H-pyrrole-3-carboxylate (70.9 mg, 0.282 mmol), K₃PO₄ (180 mg, 0.847 mmol) and Pd(dppf)Cl₂ (20.7 mg, 0.0282 mmol). The reaction was then stirred at 80° C. for 16 h under N₂. LCMS showed that the desired product was formed. The mixture was evaporated then purified by silica gel column chromatography eluting with PE:EA (0-50%) to give methyl 5-(4-(1-((tert-butylsulfinyl)amino)ethyl)naphthalen-2-yl)-1H-pyrrole-3-carboxylate (90 mg, 80%) as a white solid. LCMS (ESI): m/z=399.3, [M+H]⁺.

### Step 2. Preparation of methyl 5-(4-(1-aminoethyl)naphthalen-2-yl)-1H-pyrrole-3-carboxylate

To solution of methyl 5-(4-(1-((tert-butylsulfinyl)amino)ethyl)naphthalen-2-yl)-1H-pyrrole-3-carboxylate (90.0 mg, 0.23 mmol) in MeOH (4.0 mL) was slowly added conc. HCl (0.3 mL) at 0° C. The reaction was stirred at 20° C. for 15 h. LCMS showed that the desired product was formed. The reaction mixture was evaporated in vacuo, followed by adjusting to pH=8 with NH₃·H₂O, and then extracted with ethyl acetate (20 mL×3). The combined organic extract was dried over Na₂SO₄ and evaporated in vacuo to afford methyl 5-(4-(1-aminoethyl)naphthalen-2-yl)-1H-pyrrole-3-carboxylate (60 mg, 90%) as a yellow oil. LCMS (ESI): m/z=278.1 [M+H−NH₃]*, 296.1 [M+H−NH₃+H₂O]⁺.

### Step 3. Preparation of methyl 5-(4-(1-(5-(((tert-butoxycarbonyl)amino)methyl)-2-methylbenzamido)ethyl)naphthalen-2-yl)-1H-pyrrole-3-carboxylate

Starting from methyl 5-(4-(1-aminoethyl)naphthalen-2-yl)-1H-pyrrole-3-carboxylate (54.1 mg, 0.204 mmol) and 5-(((tert-butoxycarbonyl)amino)methyl)-2-methylbenzoic acid (60.0 mg, 0.20 mmol), methyl 5-(4-(1-(5-(((tert-butoxycarbonyl)amino)methyl)-2-methylbenzamido)ethyl) naphthalen-2-yl)-1H-pyrrole-3-carboxylate was prepared as a brown oil, using a procedure analogous to Example 4, Step 1 (100 mg, 91%). LCMS (ESI): m/z=442.3, [M+H−Boc]⁺.

### Step 4. Preparation of methyl 5-(4-(1-(5-(aminomethyl)-2-methylbenzamido)ethyl)naphthalen-2-yl)-1H-pyrrole-3-carboxylate

methyl 5-(4-(1-(5-(aminomethyl)-2-methylbenzamido)ethyl)naphthalen-2-yl)-1H-pyrrole-3-carboxylate was prepared as a white solid (21.6 mg; yield: 26.5%) from methyl 5-(4-(1-(5-(((tert-butoxycarbonyl)amino)methyl)-2-methylbenzamido)ethyl)naphthalen-2-yl)-1H-pyrrole-3-carboxylate (100.0 mg, 0.185 mmol), using a procedure analogous to the Example 4 Step 2. ¹H NMR (400 MHz, DMSO-d₆) δ=12.41-12.14 (m, 1H), 8.91 (br d, J=8.6 Hz, 1H), 8.39 (s, 1H), 8.19 (br dd, J=3.4, 6.3 Hz, 1H), 8.12 (s, 1H), 8.03 (s, 1H), 7.88 (dd, J=3.5, 6.2 Hz, 1H), 7.59 (s, 1H), 7.57-7.52 (m, 2H), 7.37 (s, 1H), 7.31 (br d, J=7.5 Hz, 1H), 7.20 (d, J=7.9 Hz, 1H), 7.04 (d, J=1.3 Hz, 1H), 5.99-5.91 (m, 1H), 3.81-3.72 (m, 5H), 2.26 (s, 3H), 1.61 (d, J=7.0 Hz, 3H). LCMS (ESI): m/z=464.0, [M+Na]⁺.

### Example 71: 5-(4-(1-(5-(aminomethyl)-2-methylbenzamido)ethyl)naphthalen-2-yl)-N-methyl-1H-pyrrole-3-carboxamide

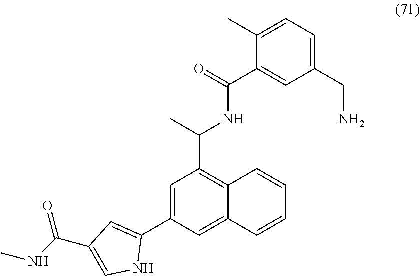 [SMILES: CNC(=O)c1c[nH]c(-c2cc(C(C)NC(=O)c3cc(CN)ccc3C)c3ccccc3c2)c1]

### Step 1. Preparation of 5-(4-(1-(5-(((tert-butoxycarbonyl)amino)methyl)-2-methylbenzamido)ethyl)naphthalen-2-yl)-1H-pyrrole-3-carboxylic acid

To a suspension of methyl 5-(4-(1-(5-(((tert-butoxycarbonyl)amino)methyl)-2-methylbenzamido)ethyl)naphthalen-2-yl)-1H-pyrrole-3-carboxylate (300.0 mg, 0.554 mmol) in MeOH (5.0 mL) and H₂O (2.0 mL) was added LiOH·H₂O (69.7 mg, 1.66 mmol). The reaction mixture was stirred at 60° C. for 16 h. LCMS showed that the desired product was formed. The reaction was concentrated in vacuo, then extracted with ethyl acetate (2 mL×2). The aqueous layer was adjusted to pH=5-6 with 1N HCl, then extracted with ethyl acetate (5 mL×2). The combined organic extract was dried over Na₂SO₄, filtered, and concentrated in vacuo to give 5-(4-(1-(5-(((tert-butoxycarbonyl)amino)methyl)-2-methylbenzamido)ethyl)naphthalen-2-yl)-1H-pyrrole-3-carboxylic acid (250 mg, 85.5%) as a white solid. LCMS (ESI): m/z=550.3, [M+Na]⁺.

### Step 2. Preparation of tert-butyl(4-methyl-3-((1-(3-(4-(methylcarbamoyl)-1H-pyrrol-2-yl)naphthalen-1-yl)ethyl)carbamoyl)benzyl)carbamate

To the stirred solution of 5-(4-(1-(5-(((tert-butoxycarbonyl)amino)methyl)-2-methylbenzamido)ethyl)naphthalen-2-yl)-1H-pyrrole-3-carboxylic acid (125.0 mg, 0.237 mmol) and MeNH₂·HCl (32.0 mg, 0.474 mmol) in DMF (2.37 mL) were added DIEA (91.9 mg, 0.711 mmol) and HATU (135 mg, 0.355 mmol) at 25° C. The reaction mixture was stirred at 25° C. for 16 h. LCMS showed that the desired product was formed. The reaction mixture was then partitioned between ethyl acetate and H₂O (10/10 mL). The organic layer was separated, and the aqueous layer was re-extracted with ethyl acetate (10 mL). The combined organic layer was washed with brine, dried with anhydrous Na₂SO₄, filtered, and evaporated in vacuo to give tert-butyl(4-methyl-3-((1-(3-(4-(methylcarbamoyl)-1H-pyrrol-2-yl)naphthalen-1-yl)ethyl)carbamoyl)benzyl) carbamate (120 mg, 93.7%) as a yellow solid, which was used in next step without further purification. LCMS (ESI): m/z=441.2, [M+H−Boc]⁺.

### Step 3. Preparation of 5-(4-(1-(5-(aminomethyl)-2-methylbenzamido)ethyl)naphthalen-2-yl)-N-methyl-1H-pyrrole-3-carboxamide

To a solution of tert-butyl(4-methyl-3-((1-(3-(4-(methylcarbamoyl)-1H-pyrrol-2-yl)naphthalen-1-yl)ethyl)carbamoyl)benzyl)carbamate (120 mg, 0.222 mmol) in CH₂Cl₂ (2 mL) was added HCl/dioxane (2 mL) at 25° C. After addition, the solution was stirred at 25° C. for 2 h. The reaction mixture was then concentrated in vacuo and purified by prep-HPLC to give 5-(4-(1-(5-(aminomethyl)-2-methylbenzamido)ethyl)naphthalen-2-yl)-N-methyl-1H-pyrrole-3-carboxamide (23.52 mg; 24.1%) as a white solid. ¹H NMR (400 MHz, DMSO-d₆) δ=11.85 (br s, 1H), 8.86 (br d, J=8.1 Hz, 1H), 8.21-8.15 (m, 1H), 8.02 (s, 1H), 7.99-7.94 (m, 1H), 7.89-7.82 (m, 2H), 7.55-7.49 (m, 2H), 7.46 (s, 1H), 7.34-7.21 (m, 2H), 7.19-7.14 (m, 1H), 7.03 (s, 1H), 5.93 (br t, J=7.4 Hz, 1H), 4.11 (br d, J=6.0 Hz, 1H), 3.72 (s, 1H), 2.73 (d, J=4.6 Hz, 3H), 2.27-2.21 (m, 3H), 1.63 (br d, J=6.8 Hz, 3H). LCMS (ESI): m/z=441.3, [M+H]⁺.

### Prep-HPLC Condition:

Column: Boston Prime C18 150*30 mm*5 μm,
Mobile phase: water (ammonium hydroxide v/v)-MeCN; B %: 25%-45%,
Flow Rate (mL/min): 35 mL/min.

The following example was prepared in a similar manner to Example 71 using an appropriate amine and an appropriate carboxylic acid.

| Ex. | Structure; | Analogous method, | |
|---|---|---|---|
| # | Compound Name | reactants | Characterizing Data |
| 72 | 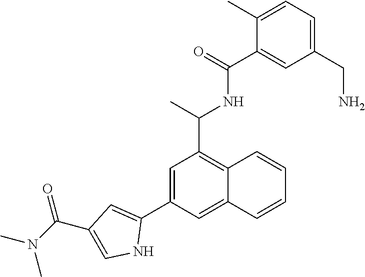 [SMILES: Cc1ccc(CN)cc1C(=O)NC(C)c1cc(-c2cc(C(=O)N(C)C)c[nH]2)cc2ccccc12] | Example 71, Step 2, using 5-(4-(1-(5-(((tert- butoxycarbonyl)amino) methyl)-2- methylbenzamido)ethyl) naphthalen-2-yl)-1H- pyrrole-3-carboxylic acid and Me₂NH•HCl; then Example 71, Step 3 | ¹H NMR (400 MHz, DMSO- d₆) δ = 11.91 (br s, 1H), 8.87 (d, J = 8.1 Hz, 1H), 8.22 − 8.15 (m, 1H), 8.08 (s, 1H), 7.99 (d, J = 1.4 Hz, 1H), 7.90 − 7.85 (m, 1H), 7.56 − 7.50 (m, 2H), 7.34 − 7.25 (m, 3H), 7.16 (d, J = 7.9 Hz, 1H), 6.92 (s, 1H), 5.93 (br t, J = 7.4 Hz, 1H), 3.71 (s, 2H), 3.10 (br s, 6H), 2.25 (s, 3H), 1.61 (d, J = 6.9 Hz, 3H). LCMS (ESI): m/z = 455.3, [M + H]⁺. |
| | 5-(4-(1-(5-(aminomethyl)-2- | | |
| | methylbenzamido)ethyl) | | |
| | naphthalen-2-yl)-N,N- | | |
| | dimethyl-1H-pyrrole-3- | | |
| | carboxamide | | |

### Example 75: 5-(aminomethyl)-2-methyl-N-(1-(3-(1-methyl-1H-pyrazol-4-yl)naphthalen-1-yl)cyclopropyl)benzamide

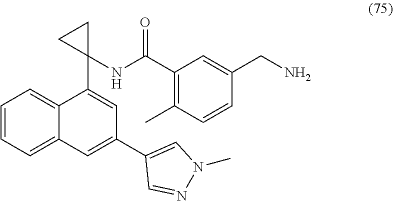 [SMILES: Cc1ccc(CN)cc1C(=O)NC1(c2cc(-c3cnn(C)c3)cc3ccccc23)CC1]

### Step 1. Preparation of 3-bromo-1-naphthamide

To the stirred solution of 3-bromo-1-naphthoic acid (2870.0 mg, 11.43 mmol) and NH₄Cl (1220 mg, 22.9 mmol) in DMF (114 mL) were added DIEA (443 Omg, 34.3 mmol) and HATU (6520 mg, 17.1 mmol) at 15° C. The reaction mixture was then stirred at 15° C. for 16 h. LCMS showed that the starting material was consumed and the desired product was observed. The reaction mixture was then poured into H₂O (200 mL) and filtered. The filter cake was washed with water and dried in vacuo to give 3-bromo-1-naphthamide (2.9 g, 101%) as a white solid. LCMS (ESI): m/z=251.8, [M+H]⁺.

### Step 2. Preparation of 3-bromo-1-naphthonitrile

To a stirred solution of 3-bromo-1-naphthamide (2900.0 mg, 11.60 mmol) in DCM (116 mL) were added TEA (4690 mg, 46.4 mmol) and trifluoroacetic anhydride (4870 mg, 23.2 mmol) at 0° C. The reaction mixture was then stirred for 16 h at 15° C. LCMS showed that the starting material was consumed. The reaction was then concentrated in vacuo and purified by silica gel chromatography using a Biotage (4 g column, PE:EA=0%-50%) to afford 3-bromo-1-naphthonitrile. ¹H NMR (400 MHz, DMSO-d₆) δ=8.65 (d, J=1.8 Hz, 1H), 8.42 (d, J=2.0 Hz, 1H), 8.11 (dd, J=4.5, 8.0 Hz, 2H), 7.88-7.75 (m, 2H).

### Step 3. Preparation of 1-(3-bromonaphthalen-1-yl)cyclopropan-1-amine

To a stirred solution of 3-bromo-1-naphthonitrile (1000 mg, 4.309 mmol) and Ti(O-iPr)₄ (1350 mg, 4.74 mmol) in Et₂O (20.0 mL) was added EtMgBr (1260 mg, 9.48 mmol) dropwise at −78° C. After that, the reaction mixture was warmed to 20° C. and stirred for 1 h. BF₃·Et₂O (1220 mg, 8.62 mmol) was then added to the reaction at 20° C., and the reaction mixture was stirred for an additional 0.5 h. LCMS showed that the starting material was consumed and the desired product was formed. The reaction was then quenched with 1N HCl (10 mL) and stirred at 20° C. for 0.5 h. The reaction mixture was added to sat. aq. NaOH (10 mL), then the solution was extracted with methyl tert-butyl ether (10 mL×2). The combined organic layer was concentrated in vacuo and purified by flash chromatography using a Biotage (20 g silica gel column, EA/PE from 0 to 50%) to afford 1-(3-bromonaphthalen-1-yl)cyclopropan-1-amine (360 mg, 31.9%) as a brown gum. ¹H NMR (400 MHz, METHANOL-d4) δ=8.41 (d, J=8.5 Hz, 1H), 8.21 (d, J=1.8 Hz, 1H), 8.10-7.86 (m, 2H), 7.82-7.60 (m, 2H), 1.65-1.47 (m, 2H), 1.40-1.34 (m, 2H). LCMS (ESI): m/z=246.9, [M+H−NH₃]⁺.

### Step 4. Preparation of tert-butyl(3-((1-(3-bromonaphthalen-1-yl)cyclopropyl)carbamoyl)-4-methylbenzyl)carbamate

Using a procedure analogous to Example 4, Step 1 with 5-(((tert-butoxycarbonyl)amino)methyl)-2-methylbenzoic acid (100.0 mg, 0.3769 mmol) and 1-(3-bromonaphthalen-1-yl)cyclopropan-1-amine (100 mg, 0.381 mmol) as the reactants affords tert-butyl(3-((1-(3-bromonaphthalen-1-yl)cyclopropyl)carbamoyl)-4-methylbenzyl)carbamate (130 mg, 68%) as a white solid. ¹H NMR (400 MHz, CHLOROFORM-d) δ=8.46 (d, J=8.4 Hz, 1H), 8.02 (d, J=2.0 Hz, 1H), 7.96 (d, J=1.8 Hz, 1H), 7.79 (d, J=8.1 Hz, 1H), 7.61-7.49 (m, 2H), 7.14 (d, J=7.3 Hz, 1H), 7.07 (d, J=7.6 Hz, 1H), 7.03 (s, 1H), 6.50 (br s, 1H), 4.76 (br s, 1H), 4.18 (br d, J=5.0 Hz, 2H), 2.17 (s, 3H), 1.45-1.37 (m, 11H).

### Step 5. Preparation of tert-butyl(4-methyl-3-((1-(3-(1-methyl-1H-pyrazol-4-yl)naphthalen-1-yl)cyclopropyl)carbamoyl)benzyl)carbamate

To a solution of tert-butyl(3-((1-(3-bromonaphthalen-1-yl)cyclopropyl)carbamoyl)-4-methylbenzyl)carbamate (130 mg, 0.255 mmol) and 1-methyl-4-(4,4,5,5-tetramethyl-1,3,2-dioxaborolan-2-yl)-1H-pyrazole (79.6 mg, 0.383 mmol) in 1,4-dioxane (2.0 mL) and H₂O (0.5 mL) were added K₃PO₄ (163 mg, 0.766 mmol) and Pd(dppf)Cl₂ (18.7 mg, 0.0255 mmol) at 20° C. The mixture was bubbled with N₂ for 1 min then stirred at 100° C. for 15 h. Subsequently, the reaction mixture was concentrated in vacuo and purified by flash chromatography (12 g silica gel column, EA/PE from 0 to 80%) to afford tert-butyl(4-methyl-3-((1-(3-(1-methyl-1H-pyrazol-4-yl)naphthalen-1-yl)cyclopropyl)carbamoyl)benzyl)carbamate (110 mg, 84%) as a yellow solid.

LCMS (ESI): m/z=533.3, [M+Na]⁺.

### Step 6. Preparation of 5-(aminomethyl)-2-methyl-N-(1-(3-(1-methyl-1H-pyrazol-4-yl)naphthalen-1-yl)cyclopropyl)benzamide

To a solution of tert-butyl(4-methyl-3-((1-(3-(1-methyl-1H-pyrazol-4-yl)naphthalen-1-yl)cyclopropyl)carbamoyl)benzyl)carbamate (110 mg, 0.215 mmol) in DCM (3.0 mL) was added TFA (491 mg, 4.31 mmol) at 20° C. The mixture was then stirred at 20° C. for 16 h. LCMS showed the starting material was consumed and the desired product was formed. After that, the reaction mixture was concentrated in vacuo to give a crude, which was dissolved in H₂O (5 mL), basified with ammonium hydroxide to pH=8-9, and extracted with ethyl acetate (5 mL×3). The combined organic layer was dried with Na₂SO₄ and filtered. The filtrate was concentrated in vacuo to afford 5-(aminomethyl)-2-methyl-N-(1-(3-(1-methyl-1H-pyrazol-4-yl)naphthalen-1-yl)cyclopropyl)benzamide (75.71 mg, 85.6%) as a brown solid. ¹H NMR (400 MHz, METHANOL-d₄) δ=8.54 (d, J=7.2 Hz, 1H), 8.18-8.13 (m, 2H), 8.02 (s, 1H), 7.99 (s, 1H), 7.96-7.87 (m, 1H), 7.57-7.46 (m, 2H), 7.34 (dd, J=1.8, 7.8 Hz, 1H), 7.24 (d, J=7.8 Hz, 1H), 7.16 (d, J=1.7 Hz, 1H), 4.02-3.95 (m, 5H), 2.15 (s, 3H), 1.50 (br s, 2H), 1.39 (br s, 2H). LCMS (ESI): m/z=411.3, [M+H]⁺.

### Examples 101-137

Examples 101-137 were all synthesized according to General Scheme 3 (GS3) where N-(1-(2-chloroquinolin-4-yl)ethyl)-2-methylbenzamide (the racemic version of P3) was used as the 2-chloroquinoline building block in every case, and the boronate building block was varied as the “variable monomer,” as in GS3. All boronic acids/esters used for these examples were commercially available in sufficient quantities. The table below describes the boronate building block used in each instance, as well as the experimental characterization data for each product. Product identities were confirmed by LCMS.

| | | | Retention | |
|---|---|---|---|---|
| | | | Time of | |
| | | | final | |
| Ex. | Structure; | Observed | compound | |
| # | Compound Name | MS | (min) | Variable Monomer |
| 101 | 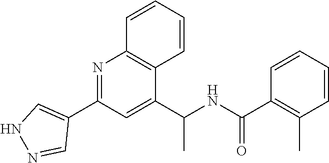 [SMILES: Cc1ccccc1C(=O)NC(C)c1cc(-c2cn[nH]c2)nc2ccccc12] | 357 | 2.061 | 1H-pyrazol-4-yl boronic acid |
| | 2-methyl-N-{1-[2-(1H-pyrazol-4- | | | |
| | yl)quinolin-4-yl]ethyl}benzamide | | | |
| 102 | 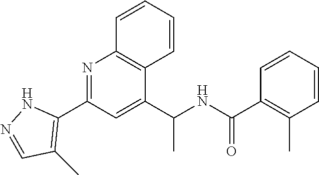 [SMILES: Cc1ccccc1C(=O)NC(C)c1cc(-c2[nH]ncc2C)nc2ccccc12] | 371 | 2.32 | 4-methyl-5-(4,4,5,5- tetramethyl-1,3,2- dioxaborolan-2-yl)- 1H-pyrazole |
| | 2-methyl-N-{1-[2-(4-methyl-1H- | | | |
| | pyrazol-5-yl)quinolin-4- | | | |
| | ylethyl}benzamide | | | |
| 103 | 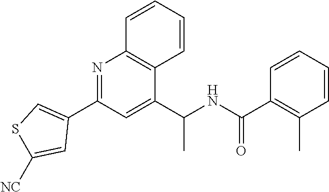 [SMILES: Cc1ccccc1C(=O)NC(C)c1cc(-c2csc(C#N)c2)nc2ccccc12] | 398 | 3.075 | (5-cyanothiophen-3- yl)boronic acid |
| | N-{1-[2-(5-cyanothiophen-3- | | | |
| | yl)quinolin-4-yl]ethyl}-2- | | | |
| | methylbenzamide | | | |
| 104 | 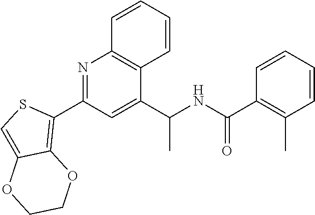 [SMILES: Cc1ccccc1C(=O)NC(C)c1cc(-c2scc3c2OCCO3)nc2ccccc12] | 431 | 2.828 | 5-(4,4,5,5- tetramethyl-1,3,2- dioxaborolan-2-yl)- 2,3-dihydrothieno [3,4-b][1,4]dioxine |
| | N-{1-[2-(2,3-dihydrothieno [3,4- | | | |
| | b][1,4]dioxin-5-yl)quinolin-4- | | | |
| | yl]ethyl}-2-methylbenzamide | | | |
| 105 | 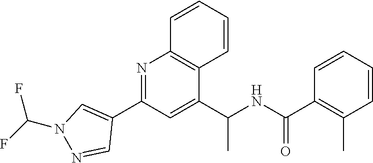 [SMILES: Cc1ccccc1C(=O)NC(C)c1cc(-c2cnn(C(F)F)c2)nc2ccccc12] | 407 | 2.589 | 1-(difluoromethyl)-4- (4,4,5,5-tetramethyl- 1,3,2-dioxaborolan-2- yl)-1H-pyrazole |
| | N-(1-{2-[1-(difluoromethyl)-1H- | | | |
| | pyrazol-4-yl]quinolin-4-yl}ethyl)-2- | | | |
| | methylbenzamide | | | |
| 106 | 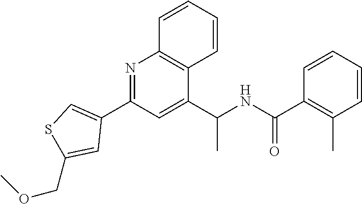 [SMILES: COCc1cc(-c2cc(C(C)NC(=O)c3ccccc3C)c3ccccc3n2)cs1] | 417 | 2.589 | 2-[5- (methoxymethyl) thiophen- 3-yl]-4,4,5,5- tetramethyl-1,3,2- dioxaborolane |
| | N-(1-{2-[5-(methoxymethyl) | | | |
| | thiophen-3-yl]quinolin-4-yl}ethyl)- | | | |
| | 2-methylbenzamide | | | |
| 107 | 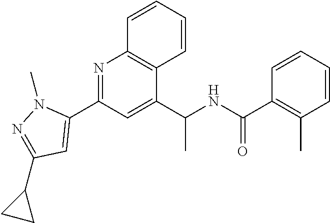 [SMILES: Cc1ccccc1C(=O)NC(C)c1cc(-c2cc(C3CC3)nn2C)nc2ccccc12] | 411 | 3.089 | 3-cyclopropyl-1- methyl-5-(4,4,5,5- tetramethyl-1,3,2- dioxaborolan-2-yl)- 1H-pyrazole |
| | N-{1-[2-(3-cyclopropyl-1-methyl- | | | |
| | 1H-pyrazol-5-yl)quinolin-4- | | | |
| | yl]ethyl}-2-methylbenzamide | | | |
| 108 | 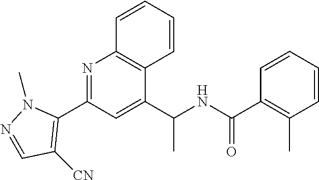 [SMILES: Cc1ccccc1C(=O)NC(C)c1cc(-c2c(C#N)cnn2C)nc2ccccc12] | 396 | 3.138 | 1-methyl-5-(4,4,5,5- tetramethyl-1,3,2- dioxaborolan-2-yl)- 1H-pyrazole-4- carbonitrile |
| | N-{1-[2-(4-cyano-1-methyl-1H- | | | |
| | pyrazol-5-yl)quinolin-4-yl]ethyl}-2- | | | |
| | methylbenzamide | | | |
| 109 | 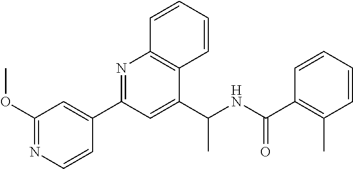 [SMILES: COc1cc(-c2cc(C(C)NC(=O)c3ccccc3C)c3ccccc3n2)ccn1] | 398 | 2.989 | (2-methoxypyridin-4- yl)boronic acid |
| | N-{1-[2-(2-methoxypyridin-4- | | | |
| | yl)quinolin-4-ylethyl}-2- | | | |
| | methylbenzamide | | | |
| 110 | 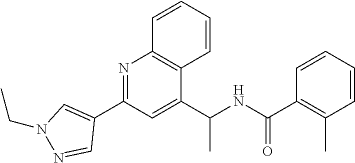 [SMILES: CCn1cc(-c2cc(C(C)NC(=O)c3ccccc3C)c3ccccc3n2)cn1] | 385 | 2.257 | 1-ethyl-4-(4,4,5,5- tetramethyl-1,3,2- dioxaborolan-2-yl)- 1H-pyrazole |
| | N-{1-[2-(1-ethyl-1H-pyrazol-4- | | | |
| | yl)quinolin-4-yl]ethyl}-2- | | | |
| | methylbenzamide | | | |
| 111 | 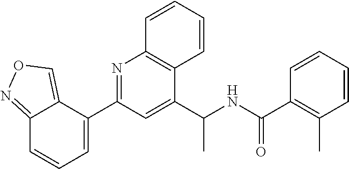 [SMILES: Cc1ccccc1C(=O)NC(C)c1cc(-c2cccc3nocc23)nc2ccccc12] | 409 | 3.383 | 5-(4,4,5,5- tetramethyl-1,3,2- dioxaborolan-2-yl)- 2,1,3-benzoxadiazole |
| | N-{1-[2-(2, 1,3-benzoxadiazol-5- | | | |
| | yl)quinolin-4-yl]ethyl}-2- | | | |
| | methylbenzamide | | | |
| 112 | 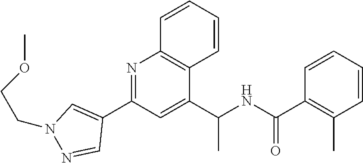 [SMILES: COCCn1cc(-c2cc(C(C)NC(=O)c3ccccc3C)c3ccccc3n2)cn1] | 415 | 2.225 | 1-(2-methoxyethyl)-4- (4,4,5,5-tetramethyl- 1,3,2-dioxaborolan-2- yl)-1H-pyrazole |
| | N-(1-{2-[1-(2-methoxyethyl)-1H- | | | |
| | pyrazol-4-yl]quinolin-4-yl}ethyl)-2- | | | |
| | methylbenzamide | | | |
| 113 | 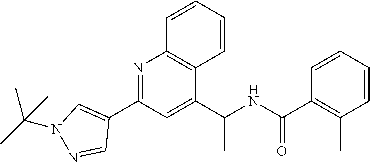 [SMILES: Cc1ccccc1C(=O)NC(C)c1cc(-c2cnn(C(C)(C)C)c2)nc2ccccc12] | 413 | 2.524 | 1-tert-butyl-4-(4,4,5,5- tetramethyl-1,3,2- dioxaborolan-2-yl)- 1H-pyrazole |
| | N-{1-[2-(1-tert-butyl-1H-pyrazol-4- | | | |
| | yl)quinolin-4-yl]ethyl}-2- | | | |
| | methylbenzamide | | | |
| 114 | 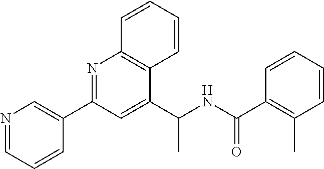 [SMILES: Cc1ccccc1C(=O)NC(C)c1cc(-c2cccnc2)nc2ccccc12] | 368 | 2.315 | pyridin-3-ylboronic acid |
| | 2-methyl-N-{1-[2-(pyridin-3- | | | |
| | yl)quinolin-4-yl]ethyl}benzamide | | | |
| 115 | 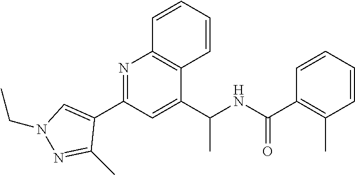 [SMILES: CCn1cc(-c2cc(C(C)NC(=O)c3ccccc3C)c3ccccc3n2)c(C)n1] | 399 | 2.312 | 1-ethyl-3-methyl-4- (4,4,5,5-tetramethyl- 1,3,2-dioxaborolan-2- yl)-1H-pyrazole |
| | N-{1-[2-(1-ethyl-3-methyl-1H- | | | |
| | pyrazol-4-yl)quinolin-4-yl]ethyl}-2- | | | |
| | methylbenzamide | | | |
| 116 | 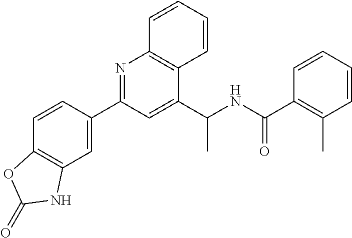 [SMILES: Cc1ccccc1C(=O)NC(C)c1cc(-c2ccc3oc(=O)[nH]c3c2)nc2ccccc12] | 424 | 2.444 | 5-(4,4,5,5- tetramethyl-1,3,2- dioxaborolan-2-yl)- 1,3-benzoxazol- 2(3H)-one |
| | 2-methyl-N-{1-[2-(2-oxo-2,3- | | | |
| | dihydro-1,3-benzoxazol-5- | | | |
| | yl)quinolin-4-yl]ethyl} benzamide | | | |
| 117 | 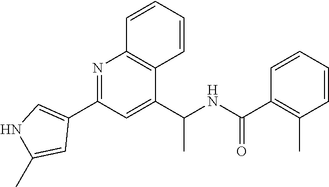 [SMILES: Cc1cc(-c2cc(C(C)NC(=O)c3ccccc3C)c3ccccc3n2)c[nH]1] | 370 | 2.351 | 2-methyl-4-(4,4,5,5- tetramethyl-1,3,2- dioxaborolan-2-yl)- 1H-pyrrole |
| | 2-methyl-N-{1-[2-(5-methyl-1H- | | | |
| | pyrrol-3-yl)quinolin-4- | | | |
| | yl]ethyl}benzamide | | | |
| 118 | 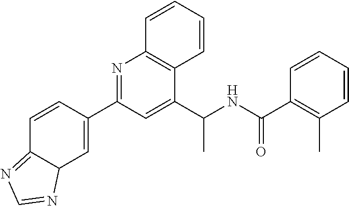 [SMILES: Cc1ccccc1C(=O)NC(C)c1cc(C2=CC3N=CN=C3C=C2)nc2ccccc12] | 408 | 3.024 | [1,2,4]triazolo[1,5- a]pyridin-6-ylboronic acid |
| | 2-methyl-N-{1-[2- | | | |
| | ([1,2,4]triazolo[1,5-a]pyridin-6- | | | |
| | yl)quinolin-4-yl]ethyl}benzamide | | | |
| 119 | 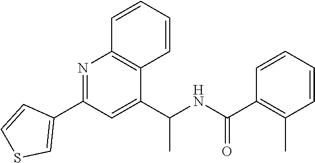 [SMILES: Cc1ccccc1C(=O)NC(C)c1cc(-c2ccsc2)nc2ccccc12] | 373 | 2.525 | thiophen-3-ylboronic acid |
| | 2-methyl-N-{1-[2-(thiophen-3- | | | |
| | yl)quinolin-4-yl]ethyl} benzamide | | | |
| 120 | 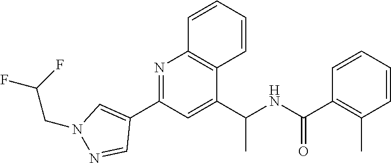 [SMILES: Cc1ccccc1C(=O)NC(C)c1cc(-c2cnn(CC(F)F)c2)nc2ccccc12] | 421 | 2.719 | 1-(2,2-difluoroethyl)- 4-(4,4,5,5- tetramethyl-1,3,2- dioxaborolan-2-yl)- 1H-pyrazole |
| | N-(1-{2-[1-(2,2-difluoroethyl)-1H- | | | |
| | pyrazol-4-yl]quinolin-4-yl}ethyl)-2- | | | |
| | methylbenzamide | | | |
| 121 | 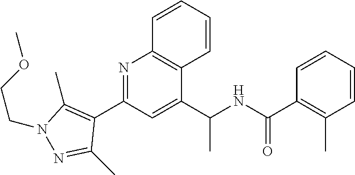 [SMILES: COCCn1nc(C)c(-c2cc(C(C)NC(=O)c3ccccc3C)c3ccccc3n2)c1C] | 443 | 2.872 | 1-(2-methoxyethyl)- 3,5-dimethyl-4- (4,4,5,5-tetramethyl- 1,3,2-dioxaborolan-2- yl)-1H-pyrazole |
| | N-(1-{2-[1-(2-methoxyethyl)-3,5- | | | |
| | dimethyl-1H-pyrazol-4-yl]quinolin- | | | |
| | 4-yl}ethyl)-2-methylbenzamide | | | |
| 122 | 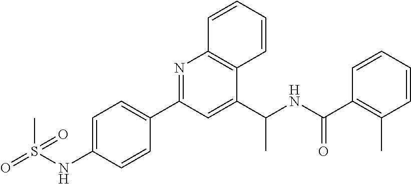 [SMILES: Cc1ccccc1C(=O)NC(C)c1cc(-c2ccc(NS(C)(=O)=O)cc2)nc2ccccc12] | 460 | 2.42 | {4-[(methanesulfonyl) amino]phenyl}boronic acid |
| | N-[1-(2-{4-[(methanesulfonyl) | | | |
| | amino]phenyl}quinolin-4-yl)ethyl]- | | | |
| | 2-methylbenzamide | | | |
| 123 | 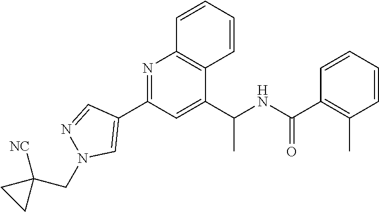 [SMILES: Cc1ccccc1C(=O)NC(C)c1cc(-c2cnn(CC3(C#N)CC3)c2)nc2ccccc12] | 436 | 2.579 | 1-{[4-(4,4,5,5- tetramethyl-1,3,2- dioxaborolan-2-yl)- 1H-pyrazol-1- yl]methyl} cyclopropane- 1-carbonitrile |
| | N-[1-(2-{1-[(1- | | | |
| | cyanocyclopropyl)methyl]-1H- | | | |
| | pyrazol-4-yl}quinolin-4-yl)ethyl]-2- | | | |
| | methylbenzamide | | | |
| 124 | 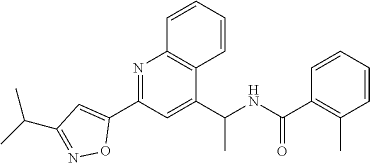 [SMILES: Cc1ccccc1C(=O)NC(C)c1cc(-c2cc(C(C)C)no2)nc2ccccc12] | 400 | 3.289 | 3-(propan-2-yl)-5- (4,4,5,5-tetramethyl- 1,3,2-dioxaborolan-2- yl)-1,2-oxazole |
| | 2-methyl-N-(1-{2-[3-(propan-2-yl)- | | | |
| | 1,2-oxazol-5-yl]quinolin-4- | | | |
| | yl}ethyl)benzamide | | | |
| 125 | 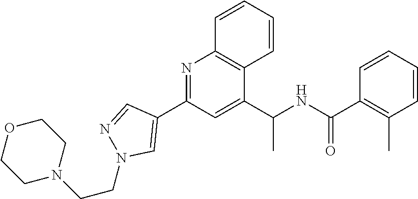 [SMILES: Cc1ccccc1C(=O)NC(C)c1cc(-c2cnn(CCN3CCOCC3)c2)nc2ccccc12] | 470 | 1.956 | 4-{2-[4-(4,4,5,5- tetramethyl-1,3,2- dioxaborolan-2-yl)- 1H-pyrazol-1- yl]ethyl}morpholine |
| | 2-methyl-N-[1-(2-{1-[2-(morpholin- | | | |
| | 4-yl)ethyl]-1H-pyrazol-4- | | | |
| | yl}quinolin-4-yl)ethyl]benzamide | | | |
| 126 | 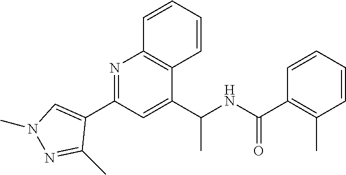 [SMILES: Cc1ccccc1C(=O)NC(C)c1cc(-c2cn(C)nc2C)nc2ccccc12] | 385 | 2.45 | 1,3-dimethyl-4- (4,4,5,5-tetramethyl- 1,3,2-dioxaborolan-2- yl)-1H-pyrazole |
| | N-{1-[2-(1,3-dimethyl-1H-pyrazol- | | | |
| | 4-yl)quinolin-4-yl]ethyl}-2- | | | |
| | methylbenzamide | | | |
| 127 | 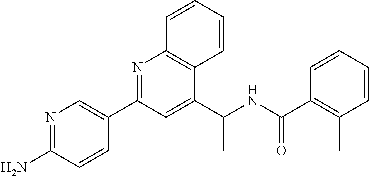 [SMILES: Cc1ccccc1C(=O)NC(C)c1cc(-c2ccc(N)nc2)nc2ccccc12] | 383 | 2.27 | (6-aminopyridin-3- yl)boronic acid |
| | N-{1-[2-(6-aminopyridin-3- | | | |
| | yl)quinolin-4-yl]ethyl}-2- | | | |
| | methylbenzamide | | | |
| 128 | 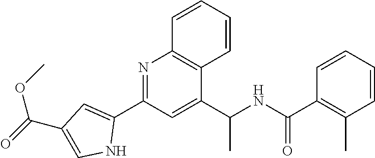 [SMILES: COC(=O)c1c[nH]c(-c2cc(C(C)NC(=O)c3ccccc3C)c3ccccc3n2)c1] | 414 | 2.963 | methyl 5-(4,4,5,5- tetramethyl-1,3,2- dioxaborolan-2-yl)- 1H-pyrrole-3- carboxylate |
| | methyl 5-{4-[1-(2- | | | |
| | methylbenzamido)ethyl]quinolin- | | | |
| | 2-yl}-1H-pyrrole-3-carboxylate | | | |
| 129 | 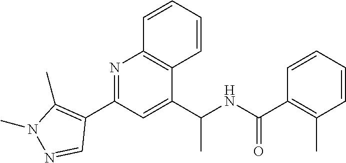 [SMILES: Cc1ccccc1C(=O)NC(C)c1cc(-c2cnn(C)c2C)nc2ccccc12] | 385 | 2.445 | 1,5-dimethyl-4- (4,4,5,5-tetramethyl- 1,3,2-dioxaborolan-2- yl)-1H-pyrazole |
| | N-{1-[2-(1,5-dimethyl-1H-pyrazol- | | | |
| | 4-yl)quinolin-4-yl]ethyl}-2- | | | |
| | methylbenzamide | | | |
| 130 | 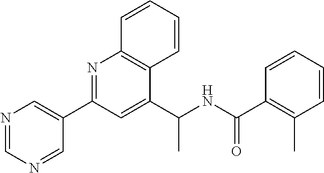 [SMILES: Cc1ccccc1C(=O)NC(C)c1cc(-c2cncnc2)nc2ccccc12] | 369 | 3.157 | pyrimidin-5-ylboronic acid |
| | 2-methyl-N-{1-[2-(pyrimidin-5- | | | |
| | yl)quinolin-4-yl]ethyl} benzamide | | | |
| 131 | 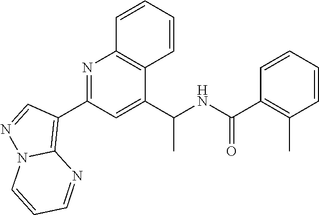 [SMILES: Cc1ccccc1C(=O)NC(C)c1cc(-c2cnn3cccnc23)nc2ccccc12] | 408 | 2.223 | 3-(4,4,5,5- tetramethyl-1,3,2- dioxaborolan-2- yl)pyrazolo[1,5- a]pyrimidine |
| | 2-methyl-N-{1-[2-(pyrazolo [1,5- | | | |
| | a]pyrimidin-3-yl)quinolin-4- | | | |
| | yl]ethyl}benzamide | | | |
| 132 | 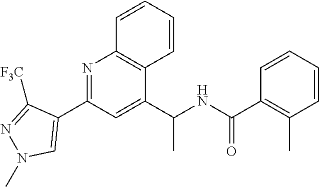 [SMILES: Cc1ccccc1C(=O)NC(C)c1cc(-c2cn(C)nc2C(F)(F)F)nc2ccccc12] | 439 | 2.837 | 1-methyl-4-(4,4,5,5- tetramethyl-1,3,2- dioxaborolan-2-yl)-3- (trifluoromethyl)-1H- pyrazole |
| | 2-methyl-N-(1-{2-[1-methyl-3- | | | |
| | (trifluoromethyl)-1H-pyrazol-4- | | | |
| | yl]quinolin-4-yl}ethyl) benzamide | | | |
| 133 | 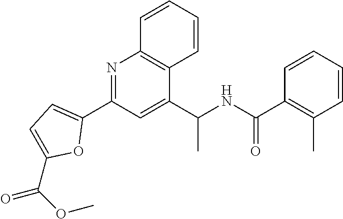 [SMILES: COC(=O)c1ccc(-c2cc(C(C)NC(=O)c3ccccc3C)c3ccccc3n2)o1] | 415 | 2.919 | methyl 5-(4,4,5,5- tetramethyl-1,3,2- dioxaborolan-2- yl)furan-2-carboxylate |
| | methyl 5-{4-[1-(2- | | | |
| | methylbenzamido)ethyl]quinolin- | | | |
| | 2-yl}furan-2-carboxylate | | | |
| 134 | 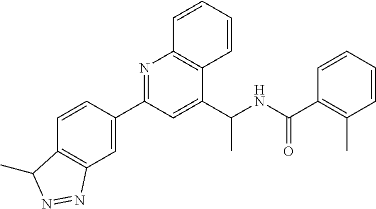 [SMILES: Cc1ccccc1C(=O)NC(C)c1cc(-c2ccc3c(c2)N=NC3C)nc2ccccc12] | 422 | 2.896 | 1-methyl-5-(4,4,5,5- tetramethyl-1,3,2- dioxaborolan-2-yl)- 1H-benzotriazole |
| | 2-methyl-N-{1-[2-(1-methyl-1H- | | | |
| | benzotriazol-5-yl)quinolin-4- | | | |
| | ylethyl}benzamide | | | |
| 135 | 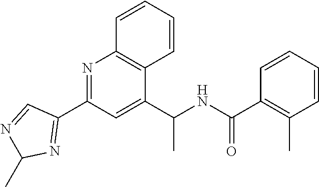 [SMILES: Cc1ccccc1C(=O)NC(C)c1cc(C2=NC(C)N=C2)nc2ccccc12] | 372 | 2.791 | 2-methyl-4-(4,4,5,5- tetramethyl-1,3,2- dioxaborolan-2-yl)- 2H-1,2,3-triazole |
| | 2-methyl-N-{1-[2-(2-methyl-2H- | | | |
| | 1,2,3-triazol-4-yl)quinolin-4- | | | |
| | yl]ethyl}benzamide | | | |
| 136 | 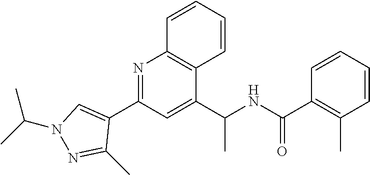 [SMILES: Cc1ccccc1C(=O)NC(C)c1cc(-c2cn(C(C)C)nc2C)nc2ccccc12] | 413 | 2.435 | 3-methyl-1-(propan-2- yl)-4-(4,4,5,5- tetramethyl-1,3,2- dioxaborolan-2-yl)- 1H-pyrazole |
| | 2-methyl-N-(1-{2-[3-methyl-1- | | | |
| | (propan-2-yl)-1H-pyrazol-4- | | | |
| | yl]quinolin-4-yl}ethyl) benzamide | | | |
| 137 | 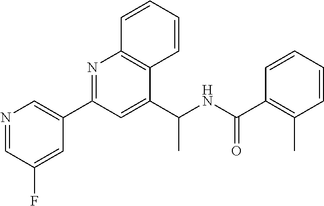 [SMILES: Cc1ccccc1C(=O)NC(C)c1cc(-c2cncc(F)c2)nc2ccccc12] | 386 | 2.949 | 3-fluoro-5-(4,4,5,5- tetramethyl-1,3,2- dioxaborolan-2- yl)pyridine |
| | N-{1-[2-(5-fluoropyridin-3- yl)quinolin-4-yl]ethyl}-2- methylbenzamide | | | |

### Examples 138-141

Examples 138-141 were all synthesized according to General Scheme 4 (GS) where N-(1-(2-chloroquinolin-4-yl)ethyl)-2-methylbenzamide (the racemic version of P3) was used as the 2-chloroquinoline building block in every case, and the amine building block of the general form N(R₁)R₂ was varied, as in GS4. All amines used for these examples were commercially available in sufficient quantities. The table below describes the amine building block used in each instance as the “variable monomer,” as well as the experimental characterization data for each product. Product identities were confirmed by LCMS.

| | | | Retention | |
|---|---|---|---|---|
| | | | Time | |
| | | | of final | |
| Ex. | | Observed | compound | |
| # | Structure; Compound Name | MS | (min) | Variable Monomer |
| 138 | 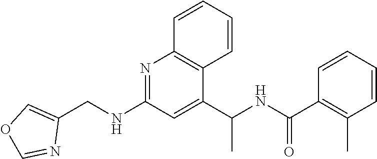 [SMILES: Cc1ccccc1C(=O)NC(C)c1cc(NCc2cocn2)nc2ccccc12] | 387 | 2.418 | 1-(1,3-oxazol-4-yl)- methanamine |
| | 2-methyl-N-[1-(2-{[(1,3-oxazol-4-yl)methyl]amino}- | | | |
| | quinolin-4-yl)ethyl]benzamide | | | |
| 139 | 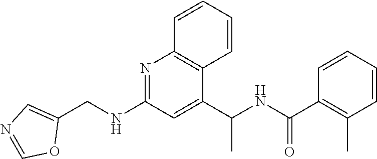 [SMILES: Cc1ccccc1C(=O)NC(C)c1cc(NCc2cnco2)nc2ccccc12] | 387 | 2.369 | 1-(1,3-oxazol-5-yl)- methanamine |
| | 2-methyl-N-[1-(2-{[(1,3-oxazol-5-yl)methyl]amino}- | | | |
| | quinolin-4-yl)ethyl]benzamide | | | |
| 140 | 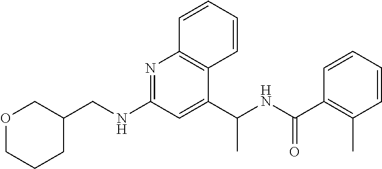 [SMILES: Cc1ccccc1C(=O)NC(C)c1cc(NCC2CCCOC2)nc2ccccc12] | 404 | 2.314 | 1-(oxan-3-yl)meth- anamine |
| | 2-methyl-N-[1-(2-{[(oxan-3-yl)methyl]amino}quinolin- | | | |
| | 4-yl)ethyl]benzamide | | | |
| 141 | 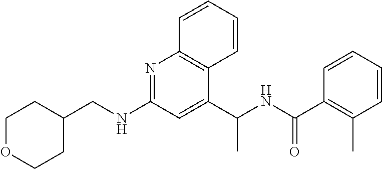 [SMILES: Cc1ccccc1C(=O)NC(C)c1cc(NCC2CCOCC2)nc2ccccc12] | 404 | 2.259 | 1-(oxan-4-yl)meth- anamine |
| | 2-methyl-N-[1-(2-{[(oxan-4-yl)methyl]amino}quinolin- | | | |
| | 4-yl)ethyl]benzamide | | | |

### Examples 142-151

Examples 142-151 were all synthesized according to General Scheme 5 (GS5) where 4-(1-(2-methylbenzamido)ethyl)quinoline 1-oxide (the racemic version of P2) was used as the N-oxide template building block in every case, and the amine building block of the general form N(R₁)R₂ was varied, as in GS5. All amines used for these examples were commercially available in sufficient quantities. The table below describes the amine building block used in each instance as the “variable monomer,” as well as the experimental characterization data for each product. Product identities were confirmed by LCMS.

| | | | Retention | |
|---|---|---|---|---|
| | | | Time | |
| | | | of final | |
| Ex. | | Observed | compound | |
| # | Structure; Compound Name | MS | (min) | Variable Monomer |
| 142 | 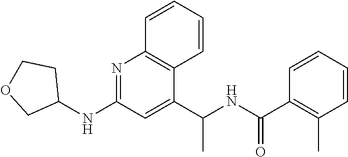 [SMILES: Cc1ccccc1C(=O)NC(C)c1cc(NC2CCOC2)nc2ccccc12] | 376 | 2.407 | oxolan-3-amine |
| | 2-methyl-N-(1-{2-[(oxolan-3-yl)amino]quinolin- | | | |
| | 4-yl}ethyl)benzamide | | | |
| 143 | 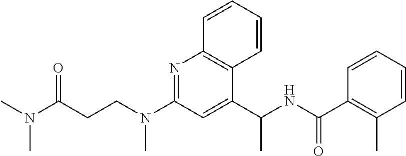 [SMILES: Cc1ccccc1C(=O)NC(C)c1cc(N(C)CCC(=O)N(C)C)nc2ccccc12] | 419 | 2.312 | 3-(dimethylamino)- N-methyl propan- amide |
| | N-[1-(2-{[3-(dimethylamino)-3-oxopropyl](methyl)amino}- | | | |
| | quinolin-4-yl)ethyl]-2-methylbenzamide | | | |
| 144 | 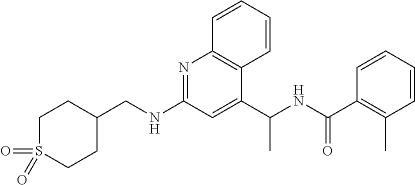 [SMILES: Cc1ccccc1C(=O)NC(C)c1cc(NCC2CCS(=O)(=O)CC2)nc2ccccc12] | 452 | 2.426 | 4-(aminomethyl)- tetrahydro-2H- thiopyran 1,1- dioxide |
| | N-(1-(2-(((1,1-dioxidotetrahydro-2H-thiopyran-4-yl)methyl)- | | | |
| | amino)quinolin-4-yl)ethyl)-2-methylbenzamide | | | |
| 145 | 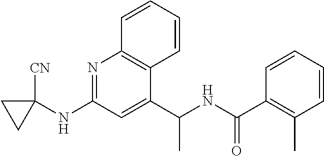 [SMILES: Cc1ccccc1C(=O)NC(C)c1cc(NC2(C#N)CC2)nc2ccccc12] | 371 | 2.475 | 1-aminocyclo- propane-1- carbonitrile |
| | N-(1-{2-[(1-cyanocyclopropyl)amino]quinolin- | | | |
| | 4-yl}ethyl)-2-methylbenzamide | | | |
| 146 | 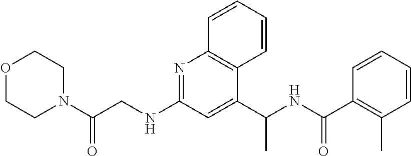 [SMILES: Cc1ccccc1C(=O)NC(C)c1cc(NCC(=O)N2CCOCC2)nc2ccccc12] | 433 | 2.38 | 2-amino-1- (morpholin-4-yl)- ethan-1-one |
| | 2-methyl-N-[1-(2-{[2-(morpholin-4-yl)-2-oxoethyl]amino}- | | | |
| | quinolin-4-yl)ethyl]benzamide | | | |
| 147 | 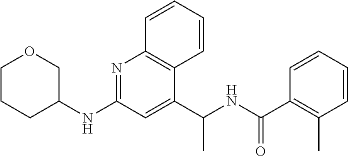 [SMILES: Cc1ccccc1C(=O)NC(C)c1cc(NC2CCCOC2)nc2ccccc12] | 390 | 2.252 | oxan-3-amine |
| | 2-methyl-N-(1-{2-[(oxan-3-yl)amino]quinolin-4- | | | |
| | ylethyl)benzamide | | | |
| 148 | 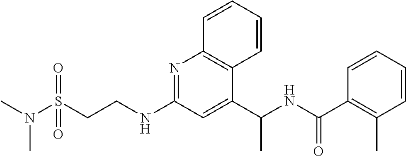 [SMILES: Cc1ccccc1C(=O)NC(C)c1cc(NCCS(=O)(=O)N(C)C)nc2ccccc12] | 441 | 2.51 | 2-amino-N,N- dimethylethane- 1-sulfonamide |
| | N-[1-(2-{[2-(dimethylsulfamoyl)ethyl]amino}quinolin- | | | |
| | 4-yl)ethyl]-2-methylbenzamide | | | |
| 149 | 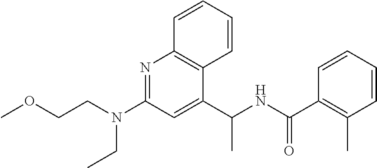 [SMILES: CCN(CCOC)c1cc(C(C)NC(=O)c2ccccc2C)c2ccccc2n1] | 392 | 2.405 | N-ethyl-2-meth- oxyethan-1-amine |
| | N-(1-{2-[ethyl(2-methoxyethyl)amino]quinolin-4- | | | |
| | yl}ethyl)-2-methylbenzamide | | | |
| 150 | 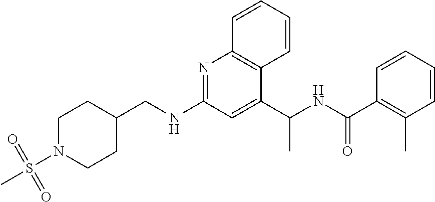 [SMILES: Cc1ccccc1C(=O)NC(C)c1cc(NCC2CCN(S(C)(=O)=O)CC2)nc2ccccc12] | 481 | 2.325 | 1-[1-(methane- sulfonyl)piperidin- 4-yl]methanamine |
| | N-{1-[2-({1-(methanesulfonyl)piperidin-4-yl]methyl}amino)- | | | |
| | quinolin-4-yl]ethyl}-2-methylbenzamide | | | |
| 151 | 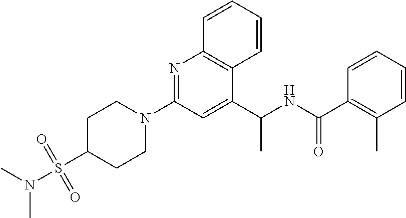 [SMILES: Cc1ccccc1C(=O)NC(C)c1cc(N2CCC(S(=O)(=O)N(C)C)CC2)nc2ccccc12] | 481 | 2.58 | N,N-dimethyl- piperidine-4- sulfonamide |
| | N-(1-{2-[4-(dimethylsulfamoyl)piperidin-1-yl]quinolin-4- | | | |
| | yl}ethyl)-2-methylbenzamide | | | |

### Examples 152-155

Examples 152-155 were all synthesized according to the first specific embodiment of General Scheme 6 (GS46) where (R)-2-methyl-N-(1-(quinolin-4-yl)ethyl)benzamide (P) was used as the quinoline template building block in every case, and the carboxylic acid building block of the general form R₁COOH was varied, as in GS5. All carboxylic acids used for these examples were commercially available in sufficient quantities. The table below describes the carboxylic acid building block used in each instance as the “variable monomer,” as well as the experimental characterization data for each product. Product identities were confirmed by LCMS.

| | | | Retention | |
|---|---|---|---|---|
| | | | Time | |
| | | | of final | |
| Ex. | | Observed | compound | |
| # | Structure; Compound Name | MS | (min) | Variable Monomer |
| 152 | 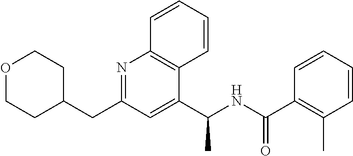 [SMILES: Cc1ccccc1C(=O)N[C@@H](C)c1cc(CC2CCOCC2)nc2ccccc12] | 389 | 2.105 | (oxan-4-yl)acetic acid |
| | 2-methyl-N-[(1R)-1-{2-[(oxan-4-yl)methyl]quinolin- | | | |
| | 4-yl}ethyl]benzamide | | | |
| 153 | 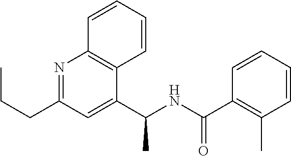 [SMILES: CCCc1cc([C@H](C)NC(=O)c2ccccc2C)c2ccccc2n1] | 333 | 2.191 | butanoic acid |
| | 2-methyl-N-[(1R)-1-(2-propylquinolin- | | | |
| | 4-yl)ethyl]benzamide | | | |
| 154 | 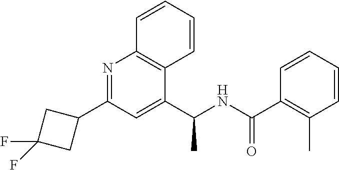 [SMILES: Cc1ccccc1C(=O)N[C@@H](C)c1cc(C2CC(F)(F)C2)nc2ccccc12] | 381 | 2.509 | 3,3-difluorocyclo- butane-1-carbox- ylic acid |
| | N-{(1R)-1-[2-(3,3-difluorocyclobutyl)quinolin- | | | |
| | 4-yllethyl}-2-methylbenzamide | | | |
| 155 | 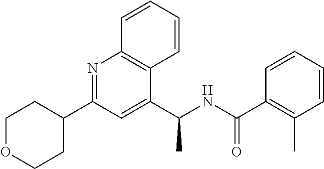 [SMILES: Cc1ccccc1C(=O)N[C@@H](C)c1cc(C2CCOCC2)nc2ccccc12] | 375 | 2.339 | oxane-4-carbox- ylic acid |
| | 2-methyl-N-{(1R)-1-[2-(oxan-4-yl)quinolin-4- | | | |
| | yl]ethyl}benzamide | | | |

### Examples 156-159

Examples 15-159 were all synthesized according to the second specific embodiment of General Scheme 6 where (R)-2-methyl-N-(1-(quinolin-4-yl)ethyl)benzamide (1) was again used as the quinoline template building block in every case, and the carboxylic acid building block of the general form R′COOH was varied, as in GS6. All carboxylic acids used for these examples were commercially available in sufficient quantities. The table below describes the carboxylic acid building block used in each instance as the “variable monomer,” as well as the experimental characterization data for each product. Product identities were confirmed by LCMS.

| | | | Retention | |
|---|---|---|---|---|
| | | | Time | |
| | | | of final | |
| Ex. | | Observed | compound | |
| # | Structure; Compound Name | MS | (min) | Variable Monomer |
| 156 | 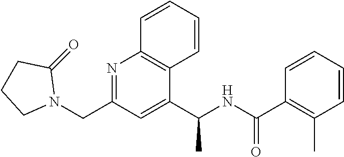 [SMILES: Cc1ccccc1C(=O)N[C@@H](C)c1cc(CN2CCCC2=O)nc2ccccc12] | 388 | 2.714 | (2-oxopyrrolidin-1- yl) acetic acid |
| | 2-methyl-N-[(1R)-1-{2-[(2-oxopyrrolidin-1-yl)- | | | |
| | methyl]quinolin-4-yl}ethyl]benzamide | | | |
| 157 | 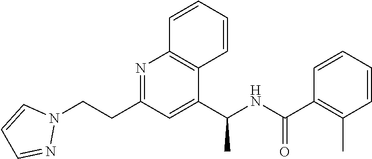 [SMILES: Cc1ccccc1C(=O)N[C@@H](C)c1cc(CCn2cccn2)nc2ccccc12] | 385 | 2.769 | 3-(1H-pyrazol-1- yl) propanoic acid |
| | 2-methyl-N-[(1R)-1-{2-[2-(1H-pyrazol-1-yl)ethyl]- | | | |
| | quinolin-4-yl}ethyl]benzamide | | | |
| 158 | 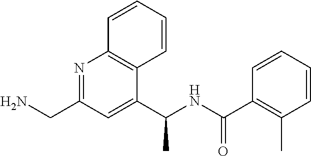 [SMILES: Cc1ccccc1C(=O)N[C@@H](C)c1cc(CN)nc2ccccc12] | 320 | 2.667 | N-(tert-butoxy- carbonyl)glycine |
| | N-{(1R)-1-[2-(aminomethyl)quinolin-4-yl]- | | | |
| | ethyl}-2-methylbenzamide | | | |
| 159 | 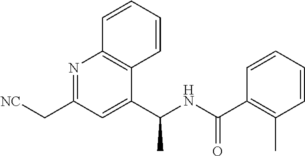 [SMILES: Cc1ccccc1C(=O)N[C@@H](C)c1cc(CC#N)nc2ccccc12] | 330 | 3.14 | cyanoacetic acid |
| | N-{(1R)-1-[2-(cyanomethyl)quinolin-4-yl]- | | | |
| | ethyl}-2-methylbenzamide | | | |

### Example 200: (R)-2-(3-methyl-4-((1-(2-(1-methyl-1H-pyrazol-4-yl)quinolin-4-yl)ethyl)carbamoyl)phenyl)acetic acid

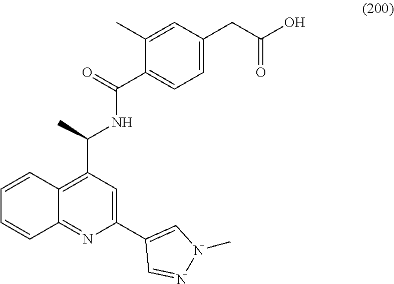 [SMILES: Cc1cc(CC(=O)O)ccc1C(=O)N[C@H](C)c1cc(-c2cnn(C)c2)nc2ccccc12]

### Step 1. Preparation of methyl 4-(2-methoxy-2-oxoethyl)-2-methylbenzoate

To a solution of methyl 4-bromo-2-methylbenzoate (10000.0 mg, 43.655 mmol) and potassium 3-ethoxy-3-oxopropanoate (20500 mg, 131 mmol) in mesitylene (300.0 mL) were added [PdCl(allyl)]₂ (1600 mg, 4.37 mmol), DMAP (5330 mg, 43.7 mmol), and CataCXium A (1570 mg, 4.37 mmol). The mixture was stirred under N₂ at 150° C. for 3 h. TLC (PE/EA=5:1, UV=254) showed new spots formed, at which time water (100 mL) was added into the reaction solution. The suspension was then extracted with ethyl acetate (100 mL×3). The combined organic layer was washed with brine (50 mL), dried with anhydrous Na₂SO₄, and evaporated in vacuo to obtain a crude solid, which was purified by flash chromatography on a silica gel column (40 g silica gel, eluted with EA) to afford methyl 4-(2-methoxy-2-oxoethyl)-2-methylbenzoate (4500 mg, 46.4%) as a yellow liquid. LCMS (ESI): m/z=223.3, [M+H]⁺.

### Step 2. Preparation of 2-(4-(methoxycarbonyl)-3-methylphenyl)acetic acid

To the solution of methyl 4-(2-methoxy-2-oxoethyl)-2-methylbenzoate (4500.0 mg, 20.25 mmol) in MeOH (45.0 mL) and H₂O (5.0 mL) at 25° C. was added LiOH·H₂O (485 mg, 20.2 mmol). The reaction was stirred at 25° C. for 16 h then evaporated in vacuo to give a residue. The residue was dissolved in H₂O (50 mL) and acidified with 1 N HCl to pH=3-4. The mixture was then extracted with ethyl acetate (50 mL×3). The combined organic layers were dried with Na₂SO₄ and evaporated in vacuo to give 2-(4-(methoxycarbonyl)-3-methylphenyl)acetic acid (3700 mg, 87.7%) as a white solid. ¹H NMR (400 MHz, CDCl₃) δ=8.00 (d, J=8.8 Hz, 1H), 7.51-7.45 (m, 2H), 3.92 (s, 3H), 3.88 (s, 2H), 2.68 (s, 3H). LCMS (ESI): m/z=209.2, [M+H]⁺.

### Step 3. Preparation of 4-(carboxymethyl)-2-methylbenzoic acid

Using a procedure similar to the one in Step 2 with NaOH instead of LiOH affords 4-(carboxymethyl)-2-methylbenzoic acid. ¹H NMR (400 MHz, DMSO-d₆) δ=12.58 (br s, 2H), 7.77 (d, J=7.8 Hz, 1H), 7.21-7.14 (m, 2H), 3.59 (s, 2H), 3.35 (br s, 3H), 1.98 (s, 1H). LCMS (ESI): m/z=195.2, [M+H]⁺.

### Step 4. Preparation of 4-(2-methoxy-2-oxoethyl)-2-methylbenzoic acid

To a solution of 4-(carboxymethyl)-2-methylbenzoic acid (3200.0 mg, 16.48 mmol) in CH₃OH (50.0 mL) was added H₂SO₄ (0.8 mL) at 25° C. The reaction mixture was stirred at 25° C. for 1 h. After that, the reaction mixture was quenched by addition of water (50 mL) and extracted with ethyl acetate (30 mL×3). The combined organic layer was washed with brine (20 mL), dried with anhydrous Na₂SO₄, filtered, and concentrated under reduced pressure to give a residue, which was then purified on a silica gel column (25 g silica gel, EA/PE=0%-30%) to give 4-(2-methoxy-2-oxoethyl)-2-methylbenzoic acid (2000 mg, 58.3%) as a white solid. ¹H NMR (400 MHz, CDCl₃) δ=8.04 (d, J=8.4 Hz, 1H), 7.22-7.19 (m, 2H), 3.71 (s, 3H), 3.65 (s, 2H), 2.65 (s, 3H). LCMS (ESI): m/z=209.1, [M+H]⁺.

### Step 5. Preparation of methyl(R)-2-(3-methyl-4-((1-(2-(1-methyl-1H-pyrazol-4-yl)quinolin-4-yl)ethyl)carbamoyl)phenyl)acetate

To a solution of 4-(2-methoxy-2-oxoethyl)-2-methylbenzoic acid (90 mg, 0.41 mmol) in DCM (4 mL) was added HATU (312 mg, 0.821 mmol) and DIEA (265 mg, 2.05 mmol) at 20° C. The reaction mixture was stirred at 20° C. for 0.5 h before (R)-1-(2-(1-methyl-1H-pyrazol-4-yl)quinolin-4-yl)ethan-1-amine (148 mg, 0.411 mmol) was added to it. The reaction mixture was stirred at 20° C. for 3 h, quenched by adding water (5 mL), and extracted with DCM (5 mL×3).

The combined organic layer was washed with brine (5 mL), dried with anhydrous Na₂SO₄, filtered, and evaporated under reduced pressure to give a residue, which was then purified by chromatography (12 g silica gel column, MeOH/DCM from 0 to 10%) to give methyl(R)-2-(3-methyl-4-((1-(2-(1-methyl-1H-pyrazol-4-yl)quinolin-4-yl)ethyl)carbamoyl)phenyl)acetate as a yellow oil. LCMS (ESI): m/z=443.2, [M+H]⁺.

### Step 6. Preparation of (R)-2-(3-methyl-4-((1-(2-(1-methyl-1H-pyrazol-4-yl)quinolin-4-yl)ethyl)carbamoyl)phenyl)acetic acid

To a solution of (R)-2-(3-methyl-4-((1-(2-(1-methyl-1H-pyrazol-4-yl)quinolin-4-yl)ethyl) carbamoyl)phenyl)acetate (380 mg, 0.43 mmol) in MeOH (3.0 mL) and H₂O (1.0 mL) at 25° C. was added LiOH·H₂O (41.1 mg, 1.72 mmol). The reaction mixture was stirred at 25° C. for 16 h then evaporated in vacuo to give a crude. The crude was then dissolved in H₂O (10 mL), acidified with 1N HCl to pH=3-4, and extracted with ethyl acetate (5 mL×3). The combined organic layer was dried with Na₂SO₄ and concentrated in vacuum to give a crude, which was purified via prep-HPLC and SFC to afford (R)-2-(3-methyl-4-((1-(2-(1-methyl-1H-pyrazol-4-yl)quinolin-4-yl)ethyl)carbamoyl)phenyl)acetic acid (10.35 mg) as a white solid after lyophilization. ¹H NMR (400 MHz, DMSO-d₆) δ=12.49-12.21 (m, 1H), 8.93 (d, J=7.7 Hz, 1H), 8.41 (s, 1H), 8.22 (d, J=7.9 Hz, 1H), 8.09 (s, 1H), 7.97 (d, J=8.4 Hz, 1H), 7.85 (s, 1H), 7.76-7.70 (m, 1H), 7.59-7.54 (m, 1H), 7.31 (d, J=7.7 Hz, 1H), 7.16-7.12 (m, 2H), 5.88 (quin, J=7.0 Hz, 1H), 3.94 (s, 3H), 3.56 (s, 2H), 2.27 (s, 3H), 1.61 (d, J=7.0 Hz, 3H): LCMS (ESI): m/z=429.3, [M+H]⁺.

### Prep-HPLC Condition:

Column: Boston Prime C18 150*25 mm*5 μm;
Mobile phase: water (ammonium hydroxide)-MeCN; B %: 35%-55%,
Flow Rate (mL/min): 35 mL/min.

### Prep-SFC Condition:

Column: DAICEL CHIRALCEL OJ (250 mm*30 mm, 10 μm);
Mobile phase: CO₂-EtOH (0.1% NH₃·H₂O); B %: 30%-30%,
Flow Rate (mL/min): 80 mL/min.

### Example 201: (R)-2-methyl-N1-(1-(2-(1-methyl-1H-pyrazol-4-yl)quinolin-4-yl)ethyl)-N4-(thiazol-4-ylmethyl)terephthalamide

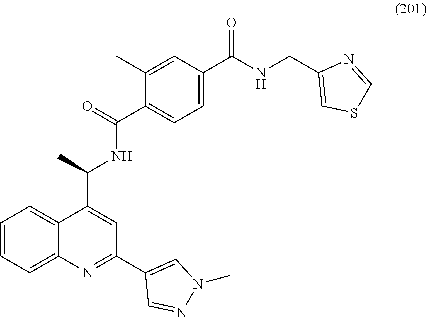 [SMILES: Cc1cc(C(=O)NCc2cscn2)ccc1C(=O)N[C@H](C)c1cc(-c2cnn(C)c2)nc2ccccc12]

To a mixture of (R)-3-methyl-4-((1-(2-(1-methyl-1H-pyrazol-4-yl)quinolin-4-yl)ethyl)carbamoyl)benzoic acid (100 mg, 0.23 mmol) in DMF (0.508 mL) and DIEA (0.3 mL), thiazol-4-ylmethanamine (27.5 mg, 0.241 mmol) and HATU (110 mg, 0.290 mmol) were added. The reaction mixture was stirred at 25° C. for 16 h, at which time, LCMS showed that the desired product was observed as the major peak. The reaction was then purified by prep-HPLC to afford (R)-2-methyl-N1-(1-(2-(1-methyl-1H-pyrazol-4-yl)quinolin-4-yl)ethyl)-N4-(thiazol-4-ylmethyl)terephthalamide (35 mg, 28%) as a white solid after evaporation in vacuo and lyophilization. ¹H NMR (400 MHz, DMSO-d₆, 296 K) δ=9.14-9.05 (m, 3H), 8.43 (s, 1H), 8.23 (d, J=8.1 Hz, 1H), 8.11 (s, 1H), 7.98 (d, J=7.7 Hz, 1H), 7.87 (s, 1H), 7.80-7.72 (m, 3H), 7.61-7.56 (m, 1H), 7.47-7.44 (m, 2H), 5.91 (t, J=7.4 Hz, 1H), 4.62 (d, J=5.7 Hz, 2H), 3.95 (s, 3H), 2.33 (s, 3H), 1.64 (d, J=7.0 Hz, 3H). LC-MS (ESI): m/z=511.4, [M+H]⁺.

### Prep-HPLC Condition:

Column: Boston Prime C18 150*30 mm*5 μm;
Mobile phase: water (ammonium hydroxide)-MeCN; B %: 30%-50%,
Flow Rate (mL/min): 35 mL/min.

### Examples 202-204, 242-246, 253-254 and 271

The following examples were prepared in a similar manner to Example 201 using an appropriate amine and an appropriate carboxylic acid.

| Ex. | | | |
|---|---|---|---|
| # | Structure; Compound Name | Reactants used | Characterizing Data |
| Ex. 202 | 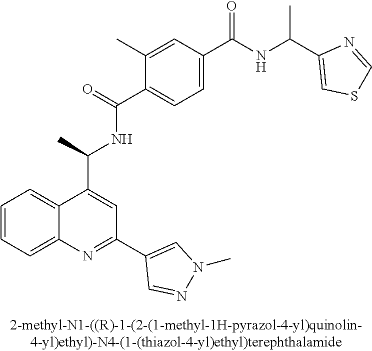 [SMILES: Cc1cc(C(=O)NC(C)c2cscn2)ccc1C(=O)N[C@H](C)c1cc(-c2cnn(C)c2)nc2ccccc12] | 1-(thiazol-4-yl)ethan-1- amine and (R)-3-methyl- 4-((1-(2-(1-methyl-1H- pyrazol-4-yl)quinolin-4- yl)ethyl)carbamoyl)- benzoic acid. | ¹H NMR (400 MHz, CDCl₃) δ ppm 8.81-8.78 (m, 1H), 8.50 (br s, 1H), 8.31 (br s, 1H), 8.20 (br d, J = 8.4 Hz, 1H), 8.08 (d, J = 7.9 Hz, 1H), 7.80-7.72 (m, 2H), 7.61-7.44 (m, 3H), 7.37-7.29 (m, 1H), 7.27-7.08 (m, 1H), 6.97 (br d, J = 8.1 Hz, 1H), 6.17-6.09 (m, 1H), 5.42 (dt, J = 3.9, 7.1 Hz, 1H), 3.96 (s, 3H), 2.41 (d, J = 7.0 Hz, 3H), 1.84 (dd, J = 1.8, 6.8 Hz, 3H), 1.60 (dd, J = 2.4, 6.8 Hz, 3H). LCMS (ESI): m/z = 525.3, [M + H]⁺. |
| Ex. 203 | 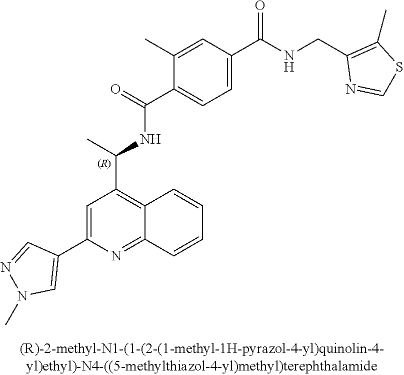 | 1-(5-methyl-1,3-thiazol- 4-yl)methanamine hydro- chloride and (R)-3-meth- yl-4-((1-(2-(1-methyl- 1H-pyrazol-4-yl)quin- olin-4-yl)ethyl)carbam- oyl)benzoic acid. Note: Before the reaction, NaHCO₃ (20.0 mg) was added to a solution of 1- (5-methyl-1,3-thiazol-4- yl)methanamine hydro- chloride (41.7 mg, 0.253 mmol) in MeOH (0.5 mL), and the mixture was stirred at 25° C. for 0.5 h. The mixture was filtered after adding CH₂Cl₂ (0.5 mL) to it. The filtrate was evaporated in vacuo, and the residue was used in the subsequent reaction. | ¹H NMR (400 MHz, MeOD- d₄) δ ppm 8.76 (s, 1H), 8.34 (s, 1H), 8.29 (d, J = 7.9 Hz, 1H), 8.18 (s, 1H), 8.08 (d, J = 8.4 Hz, 1H), 7.86 (s, 1H), 7.81-7.70 (m, 3H), 7.63 (t, J = 7.3 Hz, 1H), 7.45 (d, J = 7.8 Hz, 1H), 6.05 (q, J = 6.9 Hz, 1H), 4.67-4.60 (m, 2H), 4.01 (s, 3H), 2.57 (s, 3H), 2.39 (s, 3H), 1.76 (d, J = 7.0 Hz, 3H). LCMS (ESI): m/z = 525.3, [M + H]⁺. |
| Ex. 204 | 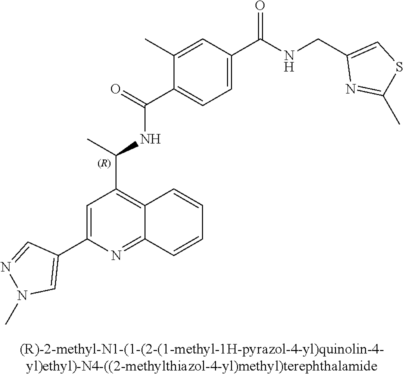 | (2-Methylthiazol-4-yl)- methanamine hydro- chloride and (R)-3-meth- yl-4-((1-(2-(1-methyl- 1H-pyrazol-4-yl)quin- olin-4-yl)ethyl)carbam- oyl)benzoic acid. Note: Before the reaction, (2- Methylthiazol-4-yl)meth- anamine hydrochloride was treated in a similar manner to 1-(5-methyl- 1,3-thiazol-4-yl)methan- amine hydrochloride in Ex. 203. | ¹H NMR (400 MHz, MeOD- d₄) δ ppm 8.34 (s, 1H), 8.29 (d, J = 8.0 Hz, 1H), 8.18 (s, 1H), 8.08 (d, J = 8.6 Hz, 1H), 7.87 (s, 1H), 7.82-7.73 (m, 3H), 7.63 (t, J = 7.3 Hz, 1H), 7.47 (d, J = 7.6 Hz, 1H), 7.20 (s, 1H), 6.06 (q, J = 6.9 Hz, 1H), 4.67-4.60 (m, 2H), 4.01 (s, 3H), 2.70 (s, 3H), 2.41 (s, 3H), 1.76 (d, J = 7.0 Hz, 3H). LCMS (ESI): m/z = 525.3, [M + H]⁺. |
| Ex. 242 | 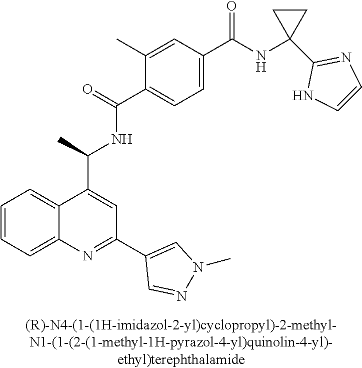 [SMILES: Cc1cc(C(=O)NC2(c3ncc[nH]3)CC2)ccc1C(=O)N[C@H](C)c1cc(-c2cnn(C)c2)nc2ccccc12] | 1-(1H-imidazol-2-yl)- cyclopropan-1-amine hydrochloride and (R)- 3-methyl-4-((1-(2-(1- methyl-1H-pyrazol-4- yl)quinolin-4-yl)ethyl)- carbamoyl)benzoic acid. Note: Before the reaction, (2-Methylthiazol-4-yl)- methanamine hydro- chloride was treated in a similar manner to 1-(5- methyl-1,3-thiazol-4-yl)- methanamine hydro- chloride in Ex. 203. | ¹H NMR (400 MHz, DMSO- d₆) δ = 11.57 (br s, 1H), 9.18- 8.98 (m, 2H), 8.42 (s, 1H), 8.23 (d, J = 8.2 Hz, 1H), 8.10 (s, 1H), 7.98 (d, J = 7.7 Hz, 1H), 7.87 (s, 1H), 7.80-7.70 (m, 3H), 7.61-7.54 (m, 1H), 7.44 (d, J = 7.8 Hz, 1H), 6.95- 6.62 (m, 2H), 5.91 (t, J = 7.2 Hz, 1H), 3.94 (s, 3H), 2.33 (s, 3H), 1.64 (d, J = 7.0 Hz, 3H), 1.40-1.32 (m, 2H), 1.20-1.13 (m, 2H). LCMS (ESI): m/z = 520.3, [M + H]⁺. |
| Ex. 243 | 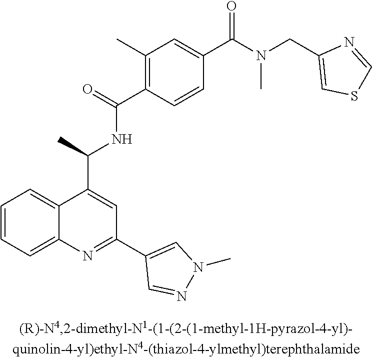 [SMILES: Cc1cc(C(=O)N(C)Cc2cscn2)ccc1C(=O)N[C@H](C)c1cc(-c2cnn(C)c2)nc2ccccc12] | N-methyl-1-(thiazol-4- yl)methanamine hydro- chloride and (R)-3-meth- yl-4-((1-(2-(1-methyl- 1H-pyrazol-4-yl)quin- olin-4-yl)ethyl)carbam- oyl)benzoic acid. | ¹H NMR (400 MHz, DMSO- d₆) δ = 9.12 (d, J = 2.0 Hz, 1H), 9.02 (br dd, J = 7.6, 16.4 Hz, 1H), 8.42 (s, 1H), 8.22 (br d, J = 8.4 Hz, 1H), 8.09 (s, 1H), 7.97 (d, J = 8.5 Hz, 1H), 7.85 (br d, J = 6.6 Hz, 1H), 7.73 (t, J = 7.8 Hz, 1H), 7.65-7.52 (m, 2H), 7.45-7.27 (m, 3H), 5.97- 5.84 (m, 1H), 4.79 (br s, 1H), 4.54 (br s, 1H), 3.93 (s, 3H), 2.91 (br d, J = 8.5 Hz, 3H), 2.36-2.20 (m, 3H), 1.62 (br s, 3H). LCMS (ESI): m/z = 525.3, [M + H]⁺. |
| Ex. 244 | 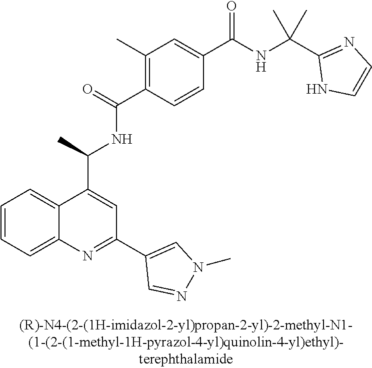 [SMILES: Cc1cc(C(=O)NC(C)(C)c2ncc[nH]2)ccc1C(=O)N[C@H](C)c1cc(-c2cnn(C)c2)nc2ccccc12] | 2-(1H-imidazol-2-yl)- propan-2-amine hydro- chloride and (R)-3- methyl-4-((1-(2-(1- methyl-1H-pyrazol-4- yl)quinolin-4-yl)ethyl) carbamoyl)benzoic acid. | ¹H NMR (400 MHz, DMSO- d₆) δ = 11.71 (br s, 1H), 9.05 (d, J = 7.8 Hz, 1H), 8.40 (d, J = 18.4 Hz, 2H), 8.23 (d, J = 8.3 Hz, 1H), 8.10 (s, 1H), 7.97 (d, J = 7.9 Hz, 1H), 7.86 (s, 1H), 7.78-7.65 (m, 3H), 7.62-7.54 (m, 1H), 7.43 (d, J = 7.9 Hz, 1H), 7.15-6.63 (m, 2H), 5.90 (quin, J = 7.2 Hz, 1H), 3.94 (s, 3H), 2.33 (s, 3H), 1.72-1.59 (m, 9H). LCMS (ESI): m/z = 522.3, [M + H]⁺. |
| Ex. 245 | 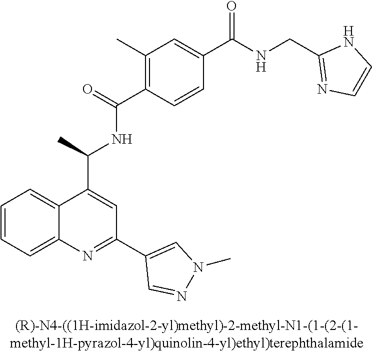 [SMILES: Cc1cc(C(=O)NCc2ncc[nH]2)ccc1C(=O)N[C@H](C)c1cc(-c2cnn(C)c2)nc2ccccc12] | (1H-imidazol-2-yl)meth- anamine hydrochloride and (R)-3-methyl-4-((1- (2-(1-methyl-1H-pyrazol- 4-yl)quinolin-4-yl)ethyl)- carbamoyl)benzoic acid. Note: Before the reaction, (1H-imidazol-2-yl)meth- anamine hydrochloride was treated in a similar manner to 1-(5-methyl- 1,3-thiazol-4-yl)meth- anamine hydrochloride in Ex. 203. | ¹H NMR (400 MHz, DMSO- d₆) δ = 11.94-11.62 (m, 1H), 9.11-8.93 (m, 2H), 8.43 (s, 1H), 8.23 (d, J = 8.2 Hz, 1H), 8.10 (s, 1H), 7.98 (dd, J = 0.7, 8.4 Hz, 1H), 7.87 (s, 1H), 7.81-7.71 (m, 3H), 7.58 (dt, J = 1.2, 7.6 Hz, 1H), 7.45 (d, J = 7.7 Hz, 1H), 7.12- 6.71 (m, 2H), 5.91 (quin, J = 7.1 Hz, 1H), 4.49 (d, J = 5.6 Hz, 2H), 3.95 (s, 3H), 2.33 (s, 3H), 1.64 (d, J = 7.0 Hz, 3H). LCMS (ESI): m/z = 494.4, [M + H]⁺. |
| Ex. 246 | 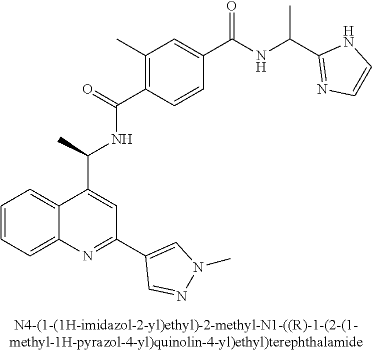 [SMILES: Cc1cc(C(=O)NC(C)c2ncc[nH]2)ccc1C(=O)N[C@H](C)c1cc(-c2cnn(C)c2)nc2ccccc12] | 1-(1H-imidazol-2-yl)- ethan-1-amine hydro- chloride and (R)-3-meth- yl-4-((1-(2-(1-methyl- 1H-pyrazol-4-yl)quin- olin-4-yl)ethyl)carbam- oyl)benzoic acid. | ¹H NMR (400 MHz, DMSO- d₆) δ = 11.77 (br s, 1H), 9.04 (d, J = 7.8 Hz, 1H), 8.76 (d, J = 7.9 Hz, 1H), 8.42 (s, 1H), 8.23 (d, J = 7.9 Hz, 1H), 8.09 (s, 1H), 8.01-7.94 (m, 1H), 7.86 (s, 1H), 7.82-7.69 (m, 3H), 7.58 (dt, J = 1.2, 7.6 Hz, 1H), 7.43 (d, J = 7.8 Hz, 1H), 7.07-6.95 (m, 1H), 6.81 (br s, 1H), 5.91 (t, J = 7.3 Hz, 1H), 5.35-5.14 (m, 1H), 3.94 (s, 3H), 2.32 (s, 3H), 1.63 (d, J = 7.0 Hz, 3H), 1.51 (d, J = 7.0 Hz, 3H). LCMS (ESI): m/z = 508.4, [M + H]⁺. |
| Ex. 253 | 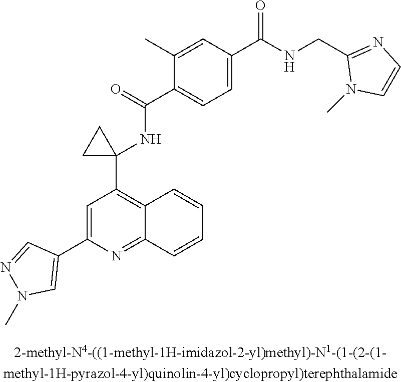 [SMILES: Cc1cc(C(=O)NCc2nccn2C)ccc1C(=O)NC1(c2cc(-c3cnn(C)c3)nc3ccccc23)CC1] | (1-methyl-1H-imidazol- 2-yl)methanamine and 3- methyl-4-((1-(2-(1-meth- yl-1H-pyrazol-4-yl)quin- olin-4-yl)cyclopropyl)- carbamoyl)benzoic acid. | ¹H NMR (400 MHz, MeOD- d₄) δ = 8.42 (d, J = 7.6 Hz, 1H), 8.29 (s, 1H), 8.15 (s, 1H), 7.98 (s, 1H), 7.95 (d, J = 8.0 Hz, 1H), 7.64 (t, J = 7.7 Hz, 1H), 7.54-7.46 (m, 3H), 7.08 (d, J = 7.9 Hz, 1H), 6.91 (d, J = 1.3 Hz, 1H), 6.76 (d, J = 1.3 Hz, 1H), 4.51-4.49 (m, 2H), 3.90 (s, 3H), 2.01 (s, 3H), 1.42- 1.31 (m, 4H). LCMS (ESI): m/z = 520.3, [M + H]⁺. |
| Ex. 254 | 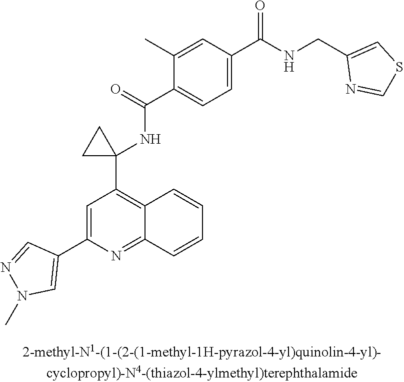 [SMILES: Cc1cc(C(=O)NCc2cscn2)ccc1C(=O)NC1(c2cc(-c3cnn(C)c3)nc3ccccc23)CC1] | Thiazol-4-yl methan- amine and 3-methyl-4- ((1-(2-(1-methyl-1H- pyrazol-4-yl)quinolin-4- yl)cyclopropyl)carbam- oyl)benzoic acid. | ¹H NMR (400 MHz, MeOD- d₄) δ = 8.85 (d, J = 2.0 Hz, 1H), 8.42 (d, J = 7.6 Hz, 1H), 8.29 (s, 1H), 8.15 (s, 1H), 7.99 (s, 1H), 7.95 (d, J = 8.0 Hz, 1H), 7.64 (t, J = 7.2 Hz, 1H), 7.57-7.47 (m, 3H), 7.33-7.29 (m, 1H), 7.09 (d, J = 7.9 Hz, 1H), 4.59 (s, 2H), 3.90 (s, 3H), 2.02 (s, 3H), 1.42-1.31 (m, 4H). LCMS (ESI): m/z = 523.3, [M + H]⁺. |
| Ex. 271 | 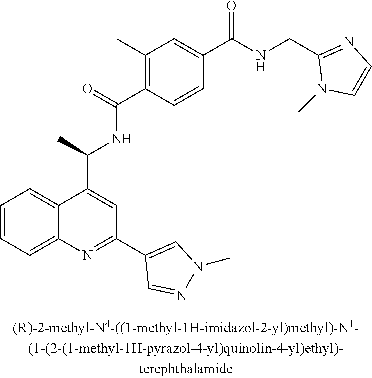 [SMILES: Cc1cc(C(=O)NCc2nccn2C)ccc1C(=O)N[C@H](C)c1cc(-c2cnn(C)c2)nc2ccccc12] | (1-methyl-1H-imidazol- 2-yl)methanamine and (R)-3-methyl-4-((1-(2- (1-methyl-1H-pyrazol-4- yl)quinolin-4-yl)ethyl)- carbamoyl)benzoic acid. | ¹H NMR (400 MHz, DMSO- d₆, 299K) Shift (ppm) = 9.11-8.96 (m, 2H), 8.43 (s, 1H), 8.27-8.21 (m, 2H), 8.10 (s, 1H), 7.98 (br d, J = 8.4 Hz, 1H), 7.86 (s, 1H), 7.81- 7.72 (m, 3H), 7.62-7.56 (m, 1H), 7.43 (d, J = 7.4 Hz, 1H), 7.09 (s, 1H), 6.80 (s, 1H), 5.94-5.87 (m, 1H), 4.52 (br d, J = 5.4 Hz, 2H), 3.94 (s, 3H), 3.65 (s, 3H), 2.31 (s, 3H), 1.63 (br d, J = 6.9 Hz, 3H). LCMS (ESI): m/z = 508.4, [M + H]⁺. |

### Examples 205-210, 235, 240, 247-248, 255, 261 and 272

The following examples were prepared in a similar manner to Example 4 using an appropriate amine and an appropriate carboxylic acid.

| Ex. | | | |
|---|---|---|---|
| # | Structure; Compound Name | Reactants used | Characterizing Data |
| Ex. 205 | 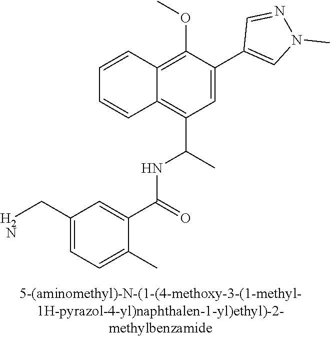 [SMILES: COc1c(-c2cnn(C)c2)cc(C(C)NC(=O)c2cc(CN)ccc2C)c2ccccc12] | Step 1, using 5-(((tert- butoxycarbonyl)amino)- methyl)-2-methyl benzoic acid and 1-(4- methoxy-3-(1-methyl- 1H-pyrazol-4-yl)naph- thalen-1-yl)ethan-1- amine; then Step 2. | ¹H NMR (400 MHz, MeOD- d₄) δ ppm 8.31-8.17 (m, 3H), 8.06 (s, 1H), 7.86 (s, 1H), 7.64-7.55 (m, 2H), 7.30 (s, 1H), 7.31 (d, J = 8.1 Hz, 1H), 7.27-7.19 (m, 1H), 6.05 (q, J = 6.7 Hz, 1H), 4.01 (s, 3H), 3.81 (s, 3H), 3.78 (s, 2H), 2.35 (s, 3H), 1.76 (d, J = 7.0 Hz, 3H). LCMS (ESI): m/z = 429, [M + H]⁺. |
| Ex. 206 | 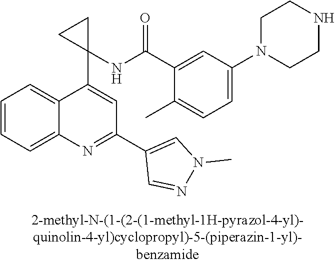 [SMILES: Cc1ccc(N2CCNCC2)cc1C(=O)NC1(c2cc(-c3cnn(C)c3)nc3ccccc23)CC1] | Step 1, using 1-(2-(1- methyl-1H-pyrazol-4- yl)quinolin-4-yl)cyclo- propan-1-amine and 5- (4-(tert-butoxycarbon- yl)piperazin-1-yl)-2- methylbenzoic acid; then Step 2. | ¹H NMR (400 MHz, MeOD- d₄) δ ppm 8.56 (d, J = 8.4 Hz, 1H), 8.41 (s, 1H), 8.27 (s, 1H), 8.12-8.05 (m, 2H), 7.76 (t, J = 7.7 Hz, 1H), 7.62 (t, J = 7.2 Hz, 1H), 7.03 (d, J = 8.5 Hz, 1H), 6.90 (d, J = 8.6 Hz, 1H), 6.68 (s, 1H), 4.04- 4.00 (m, 3H), 3.08-2.97 (m, 4H), 2.96-2.90 (m, 4H), 2.01 (s, 3H), 1.60-1.46 (m, 2H), 1.46-1.38 (m, 2H). LCMS (ESI): m/z = 467.4, [M + H]⁺. |
| Ex. 207 | 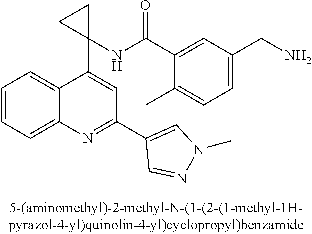 [SMILES: Cc1ccc(CN)cc1C(=O)NC1(c2cc(-c3cnn(C)c3)nc3ccccc23)CC1] | Step 1 using 1-(2-(1- methyl-1H-pyrazol-4- yl)quinolin-4-yl)cyclo- propan-1-amine and 5- (((tert-butoxycarbonyl)- amino)methyl)-2-meth- ylbenzoic acid; then Step 2. | ¹H NMR (400 MHz, MeOD- d₄) δ ppm 8.54 (d, J = 8.4 Hz, 1H), 8.48-8.41 (m, 2H), 8.27 (s, 1H), 8.13 (s, 1H), 8.07 (d, J = 8.8 Hz, 1H), 7.77 (t, J = 7.7 Hz, 1H), 7.61 (t, J = 7.6 Hz, 1H), 7.37 (d, J = 7.0 Hz, 1H), 7.29-7.25 (m, 1H), 7.18 (s, 1H), 4.03 (s, 3H), 4.02 (s, 2H), 2.16 (s, 3H), 1.55-1.44 (m, 4H). LCMS (ESI): m/z = 412, [M + H]⁺. |
| Ex. 208 | 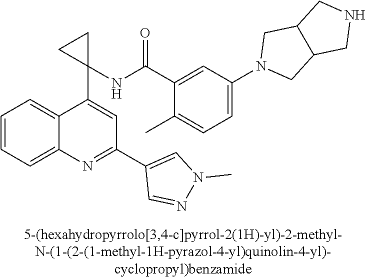 [SMILES: Cc1ccc(N2CC3CNCC3C2)cc1C(=O)NC1(c2cc(-c3cnn(C)c3)nc3ccccc23)CC1] | Step 1, using 1-(2-(1- methyl-1H-pyrazol-4- yl)quinolin-4-yl)cyclo- propan-1-amine and 5- (5-(tert-butoxycarbon- yl)hexahydropyrrolo- [3,4-c]pyrrol-2(1H)-yl)- 2-methylbenzoic acid; then Step 2. | ¹H NMR (400 MHz, MeOD- d₄) δ ppm 8.56 (d, J = 7.6 Hz, 1H), 8.48 (s, 1H), 8.41 (s, 1H), 8.27 (s, 1H), 8.10 (s, 1H), 8.07 (d, J = 8.5 Hz, 1H), 7.76 (t, J = 7.7 Hz, 1H), 7.61 (t, J = 7.3 Hz, 1H), 7.01 (d, J = 8.1 Hz, 1H), 6.69 (d, J = 7.9 Hz, 1H), 6.47 (s, 1H), 4.03 (s, 3H), 3.62-3.47 (m, 2H), 3.38-3.35 (m, 1H), 3.30- 3.08 (m, 7H), 2.05-1.98 (m, 3H), 1.54-1.48 (m, 2H), 1.48- 1.41 (m, 2H). LCMS (ESI): m/z = 493.4, [M + H]⁺. |
| Ex. 209 | 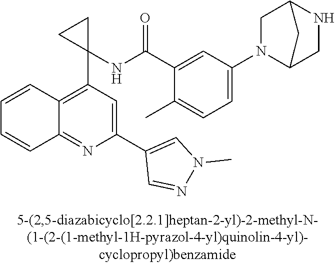 [SMILES: Cc1ccc(N2CC3CC2CN3)cc1C(=O)NC1(c2cc(-c3cnn(C)c3)nc3ccccc23)CC1] | Step 1 using 1-(2-(1- methyl-1H-pyrazol-4- yl)quinolin-4-yl)cyclo- propan-1-amine and 5- (5-(tert-butoxycarbon- yl)-2,5-diazabicyclo- [2.2.1]heptan-2-yl)-2- methylbenzoic acid; then Step 2. | ¹H NMR (400 MHz, MeOD- d₄) δ ppm 8.55 (d, J = 7.8 Hz, 1H), 8.47 (s, 1H), 8.45-8.39 (m, 1H), 8.31-8.22 (m, 1H), 8.11 (s, 1H), 8.07 (d, J = 8.6 Hz, 1H), 7.76 (t, J = 7.1 Hz, 1H), 7.67-7.56 (m, 1H), 7.04 (d, J = 8.4 Hz, 1H), 6.62 (d, J = 7.8 Hz, 1H), 6.39 (s, 1H), 4.54 (s, 1H), 4.41 (br s, 1H), 4.06-3.97 (s, 3H), 3.63 (dd, J = 2.1, 10.6 Hz, 1H), 3.25 (s, 2H), 3.16 (br d, J = 10.5 Hz, 1H), 2.23 (br d, J = 10.4 Hz, 1H), 2.06-1.95 (m, 4H), 1.55-1.49 (m, 2H), 1.48-1.42 |
| | | | (m, 2H). LCMS (ESI): m/z = |
| | | | 479.4, [M + H]⁺. |
| Ex. 210 | 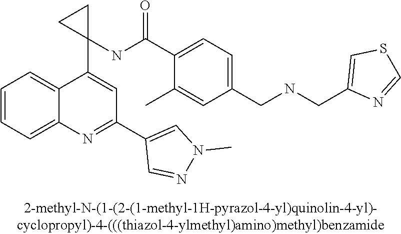 [SMILES: Cc1cc(CNCc2cscn2)ccc1C(=O)NC1(c2cc(-c3cnn(C)c3)nc3ccccc23)CC1] | Step 1 using 1-(2-(1- methyl-1H-pyrazol-4- yl)quinolin-4-yl)cyclo- propan-1-amine and 4- (((tert-butoxycarbonyl)- (thiazol-4-ylmethyl)- amino)methyl)-2-meth- ylbenzoic acid; then Step 2. | ¹H NMR (400 MHz, MeOD- d₄) δ ppm 9.06 (s, 1H), 8.55 (d, J = 8.1 Hz, 1H), 8.41 (s, 2H), 8.26 (s, 1H), 8.15-8.02 (m, 2H), 7.79-7.66 (m, 2H), 7.66-7.56 (m, 1H), 7.32-7.23 (m, 2H), 7.23-7.16 (m, 1H), 4.30 (s, 2H), 4.16 (s, 2H), 4.01 (s, 3H), 2.13 (s, 3H), 1.52 (br s, 2H), 1.49-1.42 (m, 2H). LCMS (ESI): m/z = 509.3, [M + H]⁺. |
| Ex. 235 | 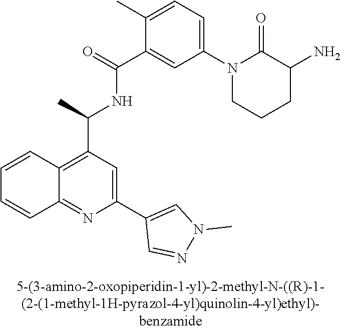 [SMILES: Cc1ccc(N2CCCC(N)C2=O)cc1C(=O)N[C@H](C)c1cc(-c2cnn(C)c2)nc2ccccc12] | Step 1, using (R)-1-(2- (1-methyl-1H-pyrazol- 4-yl)quinolin-4-yl)- ethan-1-amine and 5-(3- ((tert-butoxycarbonyl)- amino)-2-oxopiperidin- 1-yl)-2-methylbenzoic acid; then Step 2. | ¹H NMR (400 MHz, MeOD- d₄) δ = 8.35 (s, 1H), 8.28 (d, J = 7.83 Hz, 1H), 8.18-8.22 (m, 1H), 8.08 (dd, J = 8.56, 0.73 Hz, 1H), 7.88 (d, J = 1.10 Hz, 1H), 7.78 (ddd, J = 8.38, 6.97, 1.28 Hz, 1H), 7.63 (ddd, J = 8.34, 6.94, 1.22 Hz, 1H), 7.32-7.38 (m, 1H), 7.26-7.32 (m, 2H), 6.04 (q, J = 6.97 Hz, 1H), 4.02 (s, 3H), 3.71-3.85 (m, 1H), 3.60-3.70 (m, 1H), 3.49-3.59 (m, 1H), 2.39 (s, 3H), 2.23- 2.33 (m, 1H), 2.01-2.11 (m, 2H), 1.78-1.88 (m, 1H), 1.75 (dd, J = 6.97, 1.22 Hz, 3H), LCMS (ESI): m/z = 483.1, [M + H]⁺. |
| Ex. 240 | 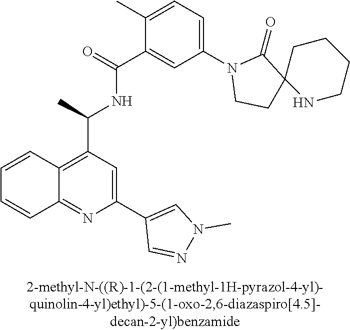 [SMILES: Cc1ccc(N2CCC3(CCCCN3)C2=O)cc1C(=O)N[C@H](C)c1cc(-c2cnn(C)c2)nc2ccccc12] | Step 1 using (R)-1-(2- (1-methyl-1H-pyrazol- 4-yl)quinolin-4-yl)- ethan-1-amine and 5- (6-(tert-butoxycarbon- yl)-1-oxo-2,6-diaza- spiro[4.5]decan-2-yl)-2- methylbenzoic acid; then Step 2. | ¹H NMR (400 MHz, MeOD- d₄) δ ppm 1.51-1.73 (m, 5H), 1.74 (d, J = 6.97 Hz, 3H), 1.85-1.97 (m, 1H), 2.09-2.21 (m, 1H), 2.34-2.38 (m, 3H), 2.39-2.46 (m, 1H), 2.68-2.80 (m, 1H), 3.05-3.18 (m, 1H), 3.80-3.90 (m, 2H), 3.99-4.07 (m, 3H), 5.95-6.14 (m, 1H), 7.28-7.35 (m, 1H), 7.50 (br d, J = 8.19 Hz, 1H), 7.60-7.67 (m, 1H), 7.78 (t, J = 7.27 Hz, 1H), 7.87-7.93 (m, 2H), 8.05- 8.11 (m, 1H), 8.22-8.27 (m, 1H), 8.27-8.32 (m, 1H), 8.36- 8.42 (m, 1H). LCMS (ESI): m/z = 523.3, [M + H]⁺. |
| Ex. 247 | 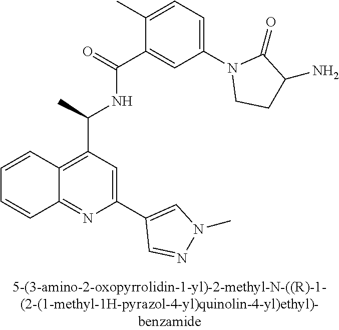 [SMILES: Cc1ccc(N2CCC(N)C2=O)cc1C(=O)N[C@H](C)c1cc(-c2cnn(C)c2)nc2ccccc12] | Step 1 using (R)-1-(2- (1-methyl-1H-pyrazol- 4-yl)quinolin-4-yl)- ethan-1-amine and 5- (3-((tert-butoxycarbon- yl)amino)-2-oxopyrrol- idin-1-yl)-2-methyl- benzoic acid then Step 2. | ¹H NMR (400 MHz, MeOD- d₄, 298K) δ (ppm) = 8.32- 8.25 (m, 1H), 8.19-8.10 (m, 2H), 7.99-7.92 (m, 1H), 7.86-7.73 (m, 2H), 7.70-7.61 (m, 1H), 7.55-7.46 (m, 1H), 7.42-7.31 (m, 1H), 7.25-7.11 (m, 1H), 6.01-5.83 (m, 1H), 3.91 (s, 3H), 3.77-3.69 (m, 2H), 3.65-3.52 (m, 1H), 2.52- 2.38 (m, 1H), 2.30-2.20 (m, 3H), 1.89-1.73 (m, 1H), 1.66- 1.58 (m, 3H) LCMS (ESI): m/z = 469.4, [M + H]⁺. |
| Ex. 248 | 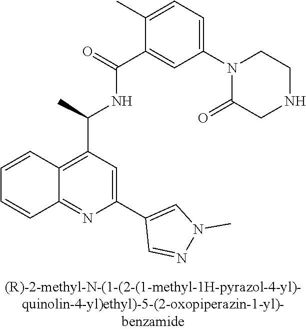 [SMILES: Cc1ccc(N2CCNCC2=O)cc1C(=O)N[C@H](C)c1cc(-c2cnn(C)c2)nc2ccccc12] | Step 1 using (R)-1-(2- (1-methyl-1H-pyrazol- 4-yl)quinolin-4-yl)- ethan-1-amine and 5- (4-(tert-butoxycarbon- yl)-2-oxopiperazin-1- yl)-2-methylbenzoic acid; then Step 2. | ¹H NMR (400 MHz, DMSO- d₆) δ = 9.01 (d, J = 7.7 Hz, 1H), 8.42 (s, 1H), 8.21 (d, J = 8.4 Hz, 1H), 8.09 (s, 1H), 7.97 (d, J = 7.7 Hz, 1H), 7.84 (s, 1H), 7.77-7.68 (m, 1H), 7.57 (t, J = 7.7 Hz, 1H), 7.37 (d, J = 1.8 Hz, 1H), 7.32-7.22 (m, 2H), 5.87 (t, J = 7.5 Hz, 1H), 3.94 (s, 3H), 3.60 (t, J = 5.4 Hz, 2H), 3.40 (s, 2H), 3.01 (t, J = 5.4 Hz, 2H), 2.27 (s, 3H), 1.61 (d, J = 7.0 Hz, 3H). LCMS (ESI): m/z = 469.4, [M + H]⁺. |
| Ex. 255 | 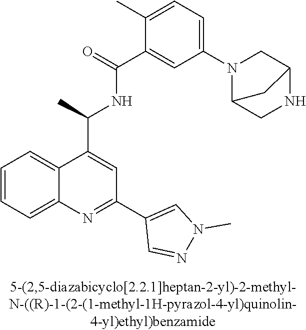 [SMILES: Cc1ccc(N2CC3CC2CN3)cc1C(=O)N[C@H](C)c1cc(-c2cnn(C)c2)nc2ccccc12] | Step 1, using (R)-1-(2- (1-methyl-1H-pyrazol- 4-yl)quinolin-4-yl)- ethan-1-amine and 5- (5-(tert-butoxycarbon- yl)-2,5-diazabicyclo- [2.2.1]heptan-2-yl)-2- methylbenzoic acid; then Step 2. | ¹H NMR (400 MHz, MeOD- d₄) δ = 8.33 (d, J = 2.3 Hz, 1H), 8.27 (d, J = 8.4 Hz, 1H), 8.16 (d, J = 3.9 Hz, 1H), 8.06 (d, J = 8.3 Hz, 1H), 7.87 (d, J = 2.8 Hz, 1H), 7.77 (t, J = 7.6 Hz, 1H), 7.62 (t, J = 7.7 Hz, 1H), 7.07 (d, J = 8.4 Hz, 1H), 6.65-6.56 (m, 2H), 6.01 (q, J = 6.7 Hz, 1H), 4.40 (d, J = 7.8 Hz, 1H), 4.00 (s, 3H), 3.91 (s, 1H), 3.59 (td, J = 2.6, 9.4 Hz, 1H), 3.08-2.97 m, 3H), 2.22 (s, 3H), 2.02 (d, J = 12.0 Hz, 1H), 1.84 (d, J = 10.6 Hz, 1H), 1.73 (d, J = 7.0 Hz, 3H). LCMS (ESI): m/z = 467.4 [M + H]⁺. |
| Ex. 261 | 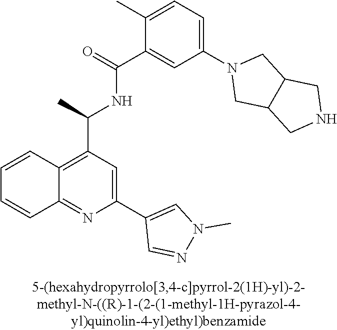 [SMILES: Cc1ccc(N2CC3CNCC3C2)cc1C(=O)N[C@H](C)c1cc(-c2cnn(C)c2)nc2ccccc12] | Step 1 using (R)-1-(2- (1-methyl-1H-pyrazol- 4-yl)quinolin-4-yl)- ethan-1-amine and 5- (5-(tert-butoxycarbon- yl)hexahydropyrrolo- [3,4-c] pyrrol-2(1H)- yl)-2-methylbenzoic acid then Step 2. | ¹H NMR (400 MHz, DMSO- d₆) δ ppm 1.06 (s, 1H), 1.17- 1.29 (m, 1H), 1.63-1.72 (m, 3H), 2.12-2.21 (m, 3H), 2.97- 3.16 (m, 4H), 3.22-3.42 (m, 5H), 3.43-3.51 (m, 3H), 4.00- 4.05 (m, 3H), 5.91-6.04 (m, 1H), 6.61-6.78 (m, 2H), 7.01- 7.09 (m, 1H), 7.81-7.94 (m, 1H), 8.03-8.12 (m, 1H), 8.28- 8.40 (m, 1H), 8.50-8.71 (m, 3H), 9.03-9.14 (m, 2H), 9.19- 9.40 (m, 2H). LCMS (ESI): m/z = 481.4 [M + H]⁺. |
| Ex. 272 | 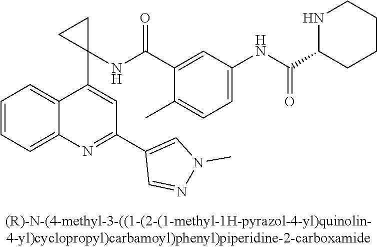 [SMILES: Cc1ccc(NC(=O)[C@H]2CCCCN2)cc1C(=O)NC1(c2cc(-c3cnn(C)c3)nc3ccccc23)CC1] | Step 1, using 1-(2-(1- methyl-1H-pyrazol-4- yl)quinolin-4-yl)cyclo- propan-1-amine and (R)-5-(1-(tert-butoxy- carbonyl)piperidine-2- carboxamido)-2-meth- ylbenzoic acid acid then Step 2. | ¹H NMR (400 MHz, MeOD- d₄) δ ppm 8.56 (d, J = 8.0 Hz, 1H), 8.41 (s, 1H), 8.27 (s, 1H), 8.11 (s, 1H), 8.07 (d, J = 8.4 Hz, 1H), 7.76 (t, J = 7.3 Hz, 1H), 7.62 (t, J = 7.3 Hz, 1H), 7.43 (d, J = 7.9 Hz, 1H), 7.39 (s, 1H), 7.11 (d, J = 8.4 Hz, 1H), 4.05-4.00 (m, 3H), 3.31-3.21 (m, 1H), 3.09 (br d, J = 13.0 Hz, 1H), 2.69-2.61 (m, 1H), 2.07 (s, 3H), 1.90 (br d, J = 9.5 Hz, 2H), 1.62 (br d, J = 11.0 Hz, 1H), 1.58- 1.42 (m, 7H). LCMS (ESI): m/z = 509.2, [M + H]⁺. |

### Example 211: 2-methyl-N-(1-(2-(1-methyl-1H-pyrazol-4-yl)quinolin-4-yl)cyclopropyl)-5-(4-methylpiperazin-1-yl)benzamide

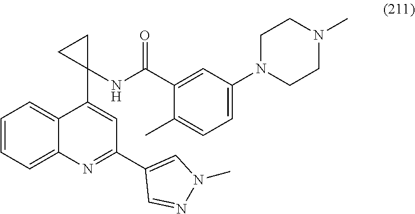 [SMILES: Cc1ccc(N2CCN(C)CC2)cc1C(=O)NC1(c2cc(-c3cnn(C)c3)nc3ccccc23)CC1]

To a solution of 2-methyl-5-(4-methylpiperazin-1-yl)benzoic acid (133 mg, 0.492 mmol) in DMF (10 mL) were added HATU (216 mg, 0.567 mmol) and TEA (1.0 mL), followed by 1-(2-(1-methyl-1H-pyrazol-4-yl)quinolin-4-yl)cyclopropan-1-amine (100.0 mg, 0.378 mmol) at 20° C. The reaction mixture was stirred at 20° C. for 3 h. LCMS showed a mass peak of the desired product. The reaction mixture was partitioned between ethyl acetate and H₂O (100/50 mL). The organic layer was separated, and the aqueous layer was re-extracted with ethyl acetate (50 mL). The combined organic layer was washed with brine, dried with anhydrous Na₂SO₄, and evaporated in vacuo to afford a crude product, which was then dissolved in 5 mL DMF and purified by prep-HPLC to afford 2-methyl-N-(1-(2-(1-methyl-1H-pyrazol-4-yl)quinolin-4-yl)cyclopropyl)-5-(4-methylpiperazin-1-yl)benzamide (30 mg, 15%) as a yellow solid after lyophilization. ¹H NMR (400 MHz, MeOD-d₄) δ ppm 8.55 (d, J=8.4 Hz, 1H), 8.48-8.37 (m, 2H), 8.27 (s, 1H), 8.10 (s, 1H), 8.07 (d, J=8.7 Hz, 1H), 7.76 (t, J=7.2 Hz, 1H), 7.61 (t, J=7.6 Hz, 1H), 7.06 (d, J=8.6 Hz, 1H), 6.94 (d, J=7.9 Hz, 1H), 6.71 (s, 1H), 4.03 (s, 3H), 3.29-3.17 (m, 4H), 3.10-2.98 (m, 4H), 2.701 (s, 3H), 2.02 (s, 3H), 1.55-1.48 (m, 2H), 1.47-1.40 (m, 2H). LCMS (ESI): m/z=481, [M+H]⁺.

### Prep-HPLC Condition:

Column: Boston Prime C18 150*30 mm*5 μm;
Mobile phase: water (FA)-MeCN; B %: 0%-30%,
Flow Rate (mL/min): 30 mL/min.

### Examples 212-215, 228, 241 and 269-270

The following examples were prepared in a similar manner to Example 211 using an appropriate amine and an appropriate carboxylic acid.

| Ex. | Structure; | | |
|---|---|---|---|
| # | Compound Name | Reactants used | Characterizing Data |
| Ex. 212 | 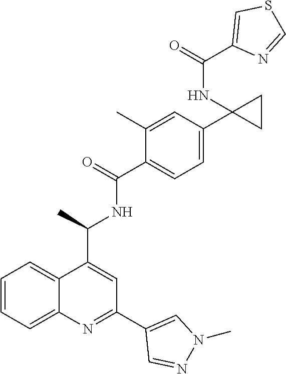 [SMILES: Cc1cc(C2(NC(=O)c3cscn3)CC2)ccc1C(=O)N[C@H](C)c1cc(-c2cnn(C)c2)nc2ccccc12] | (R)-1-(2-(1-methyl-1H- pyrazol-4-yl)quinolin- 4-yl)ethan-1-amine and 2-methyl-4-(1- (thiazole-4- carboxamido) cyclopropyl)benzoic acid. | ¹H NMR (400 MHz, MeOD-d₄) δ ppm 9.04 (s, 1H), 8.34 − 8.24 (m, 3H), 8.21 − 8.13 (m, 1H), 8.07 (d, J = 7.9 Hz, 1H), 7.85 (s, 1H), 7.77 (t, J = 7.7 Hz, 1H), 7.62 (t, J = 7.3 Hz, 1H), 7.36 − 7.30 (m, 1H), 7.20 − 7.14 (m, 2H), 6.02 (q, J = 6.9 Hz, 1H), 4.01 (s, 3H), 2.33 (s, 3H), 1.73 (d, J = 7.0 Hz, 3H), 1.45 − 1.40 (m, 2H), 1.40 − 1.34 (m, 2H). LCMS (ESI): m/z = 537, [M + H]⁺. |
| | (R)-N-(1-(3-methyl-4-((1-(2- | | |
| | (1-methyl-1H-pyrazol-4- | | |
| | yl)quinolin-4-yl)ethyl) | | |
| | carbamoyl)phenyl) | | |
| | cyclopropyl)thiazole-4- | | |
| | carboxamide | | |
| Ex. 213 | 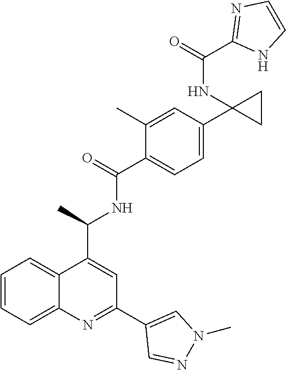 [SMILES: Cc1cc(C2(NC(=O)c3ncc[nH]3)CC2)ccc1C(=O)N[C@H](C)c1cc(-c2cnn(C)c2)nc2ccccc12] | (R)-1-(2-(1-methyl-1H- pyrazol-4-yl)quinolin- 4-yl)ethan-1-amine and 4-(1-(1H- imidazole-2- carboxamido)cyclopro pyl)-2-methylbenzoic acid. | ¹H NMR (400 MHz, MeOD-d₄) δ ppm 8.33 (s, 1H), 8.27 (d, J = 8.0 Hz, 1H), 8.17 (s, 1H), 8.07 (d, J = 8.6 Hz, 1H), 7.85 (s, 1H), 7.77 (t, J = 7.6 Hz, 1H), 7.62 (t, J = 7.2 Hz, 1H), 7.39 − 7.28 (m, 1H), 7.25 − 7.12 (m, 4H), 6.02 (q, J = 7.0 Hz, 1H), 4.01 (s, 3H), 2.33 (s, 3H), 1.73 (d, J = 7.1 Hz, 3H), 1.40 (s, 2H), 1.38 (s, 2H). LCMS (ESI): m/z = 520.3, [M + H]⁺. |
| | (R)-N-(1-(3-methyl-4-((1-(2- | | |
| | (1-methyl-1H-pyrazol-4- | | |
| | yl)quinolin-4- | | |
| | yl)ethyl)carbamoyl)phenyl) | | |
| | cyclopropyl)-1H-imidazole- | | |
| | 2-carboxamide | | |
| Ex. 214 | 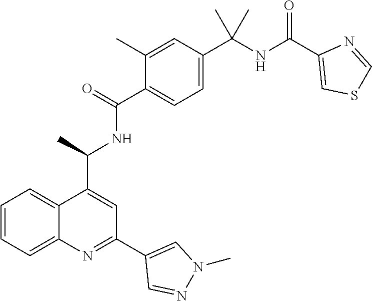 [SMILES: Cc1cc(C(C)(C)NC(=O)c2cscn2)ccc1C(=O)N[C@H](C)c1cc(-c2cnn(C)c2)nc2ccccc12] | (R)-1-(2-(1-methyl-1H- pyrazol-4-yl)quinolin- 4-yl)ethan-1-amine and 2-methyl-4-(2- (thiazole-4- carboxamido)propan- 2-yl)benzoic acid. | ¹H NMR (400 MHz, MeOD-d₄) δ ppm 9.07 − 9.03 (m, 1H), 8.34 (s, 1H), 8.28 (d, J = 8.5 Hz, 1H), 8.18 (s, 1H), 8.17 (s, 1H), 8.07 (d, J = 7.9 Hz, 1H), 7.87 (s, 1H), 7.78 (t, J = 7.2 Hz, 1H), 7.63 (t, J = 7.7 Hz, 1H), 7.40 − 7.32 (m, 3H), 6.02 (q, J = 7.0 Hz, 1H), 4.01 (s, 3H), 2.35 (s, 3H), 1.79 (s, 6H), 1.73 (d, J = 6.8 Hz, 3H). LCMS (ESI): m/z = 539, [M + H]⁺. |
| | (R)-N-(2-(3-methyl-4-((1-(2- | | |
| | (1-methyl-1H-pyrazol-4- | | |
| | yl)quinolin-4-yl)ethyl) | | |
| | carbamoyl)phenyl)propan- | | |
| | 2-yl)thiazole-4-carboxamide | | |
| Ex. 215 | 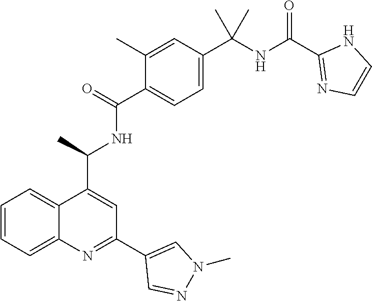 [SMILES: Cc1cc(C(C)(C)NC(=O)c2ncc[nH]2)ccc1C(=O)N[C@H](C)c1cc(-c2cnn(C)c2)nc2ccccc12] | (R)-1-(2-(1-methyl-1H- pyrazol-4-yl)quinolin- 4-yl)ethan-1-amine and 4-(2-(1H- imidazole-2- carboxamido)propan- 2-yl)-2-methylbenzoic acid. | ¹H NMR (400 MHz, MeOD-d₄) ppm 8.34 (s, 1H), 8.28 (d, J = 8.0 Hz, 1H), 8.21 − 8.14 (m, 1H), 8.07 (d, J = 8.4 Hz, 1H), 7.87 (s, 1H), 7.78 (t, J = 7.3 Hz, 1H), 7.63 (t, J = 7.3 Hz, 1H), 7.39 − 7.32 (m, 3H), 7.21 − 7.18 (m, 2H), 6.03 (q, J = 6.9 Hz, 1H), 4.01 (s, 3H), 2.36 (s, 3H), 1.77 (s, 6H), 1.73 (d, J = 7.0 Hz, 3H). LCMS (ESI): m/z = 522.0, [M + H]⁺. |
| | (R)-N-(2-(3-methyl-4-((1-(2- | | |
| | (1-methyl-1H-pyrazol-4- | | |
| | yl)quinolin-4-yl)ethyl) | | |
| | carbamoyl)phenyl)propan- | | |
| | 2-yl)-1H-imidazole-2- | | |
| | carboxamide | | |
| Ex. 228 | 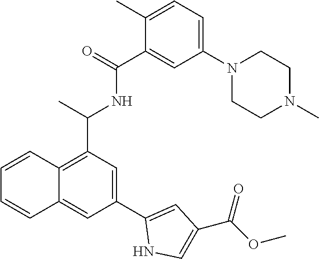 [SMILES: COC(=O)c1c[nH]c(-c2cc(C(C)NC(=O)c3cc(N4CCN(C)CC4)ccc3C)c3ccccc3c2)c1] | Methyl 5-(4-(1- aminoethyl)naphthalen- 2-yl)-1H-pyrrole-3- carboxylate and 2- methyl-5-(4- methylpiperazin-1- yl)benzoic acid | ¹H NMR (400 MHz, DMSO-d₆) = 12.20 (br s, 1H), 8.87 (d, J = 8.4 Hz, 1H), 8.18 (dd, J = 3.5, 5.9 Hz, 1H), 8.10 (s, 1H), 8.03 (s, 1H), 7.88 (dd, J = 3.3, 6.4 Hz, 1H), 7.59 (dd, J = 1.5, 2.9 Hz, 1H), 7.56 − 7.51 (m, 2H), 7.08 − 7.02 (m, 2H), 6.93 − 6.84 (m, 2H), 5.92 (s, 1H), 3.74 (s, 3H), 3.08 (br d, J = 3.1 Hz, 4H), 2.41 (t, J = 4.7 Hz, 4H), 2.20 (s, 3H), 2.17 (s, 3H), 1.59 (d, J = 6.8 Hz, 3H). LCMS (ESI): m/z = 511.4, [M + H]⁺. |
| | methyl 5-(4-(1-(2-methyl-5- | | |
| | (4-methylpiperazin-1- | | |
| | yl)benzamido)ethyl) | | |
| | naphthalen-2-yl)-1H- | | |
| | pyrrole-3-carboxylate | | |
| Ex. 241 | 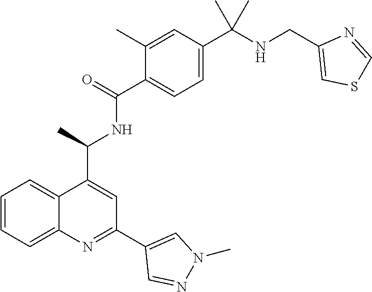 [SMILES: Cc1cc(C(C)(C)NCc2cscn2)ccc1C(=O)N[C@H](C)c1cc(-c2cnn(C)c2)nc2ccccc12] | (R)-1-(2-(1-methyl-1H- pyrazol-4-yl)quinolin- 4-yl)ethan-1-amine and 2-methyl-4-(2- ((thiazol-4- ylmethyl)amino) propan-2-yl)benzoic acid | ¹H NMR (400 MHz, MeOD-d₄) δ ppm 9.01 (s, 1H), 8.35 (s, 1H), 8.29 (d, J = 7.9 Hz, 1H), 8.18 (s, 1H), 8.08 (d, J = 8.4 Hz, 1H), 7.87 (s, 1H), 7.79 (t, J = 7.7 Hz, 1H), 7.68 − 7.40 (m, 5H), 6.05 (q, J = 6.9 Hz, 1H), 4.02 (s, 3H), 3.83 (s, 2H), 2.41 (s, 3H), 1.76 (d, J = 7.0 Hz, 3H), 1.70 (s, 6H). LCMS (ESI): m/z = 525.3, [M + H]⁺. |
| | (R)-2-methyl-N-(1-(2-(1- | | |
| | methyl-1H-pyrazol-4- | | |
| | yl)quinolin-4-yl)ethyl)-4-(2- | | |
| | ((thiazol-4- | | |
| | ylmethyl)amino)propan-2- | | |
| | yl)benzamide | | |
| Ex. 269 | 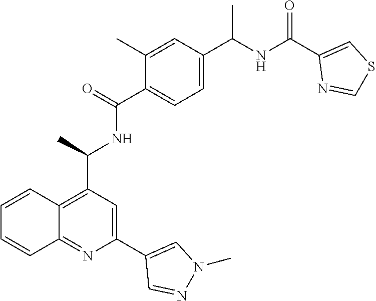 [SMILES: Cc1cc(C(C)NC(=O)c2cscn2)ccc1C(=O)N[C@H](C)c1cc(-c2cnn(C)c2)nc2ccccc12] | Example 211, using (R)-1-(2-(1-methyl-1H- pyrazol-4-yl)quinolin- 4-yl)ethan-1-amine and 2-methyl-4-(1- (thiazole-4- carboxamido)ethyl) benzoic acid. | ¹H NMR (400 MHz, MeOD-d₄ − d4) δ = 9.02 (d, J = 1.7 Hz, 1H), 8.31 (s, 1H), 8.29 − 8.22 (m, 2H), 8.16 (d, J = 2.1 Hz, 1H), 8.06 (d, J = 8.4 Hz, 1H), 7.84 (s, 1H), 7.76 (t, J = 7.6 Hz, 1H), 7.65 − 7.58 (m, 1H), 7.39 − 7.34 (m, 1H), 7.33 − 7.28 (m, 2H), 6.02 (q, J = 6.6 Hz, 1H), 5.24 − 5.18 (m, 1H), 3.99 (d, J = 3.3 Hz, 3H), 2.35 (s, 3H), 1.72 (d, J = 7.0 Hz, 3H), 1.58 (d, J = 7.0 Hz, 3H). LCMS (ESI): m/z = 525.3, [M + H]⁺ |
| | N-(1-(3-methyl-4-(((R)-1-(2- | | |
| | (1-methyl-1H-pyrazol-4- | | |
| | yl)quinolin-4- | | |
| | yl)ethyl)carbamoyl)phenyl) | | |
| | ethyl)thiazole-4- | | |
| | carboxamide | | |
| Ex. 270 | 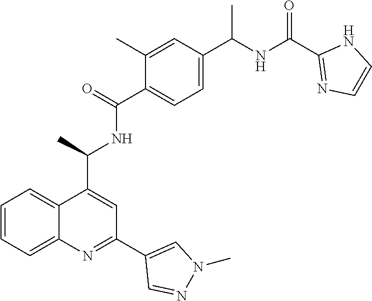 [SMILES: Cc1cc(C(C)NC(=O)c2ncc[nH]2)ccc1C(=O)N[C@H](C)c1cc(-c2cnn(C)c2)nc2ccccc12] | (R)-1-(2-(1-methyl-1H- pyrazol-4-yl)quinolin- 4-yl)ethan-1-amine and 4-(1-(1H- imidazole-2- carboxamido)ethyl)-2- methylbenzoic acid. | ¹H NMR (400 MHz, MeOD-d₄) δ = 8.32 (s, 1H), 8.27 (d, J = 7.9 Hz, 1H), 8.16 (s, 1H), 8.06 (d, J = 8.1 Hz, 1H), 7.84 (s, 1H), 7.76 (t, J = 7.5 Hz, 1H), 7.66 − 7.56 (m, 1H), 7.39 − 7.34 (m, 1H), 7.32 − 7.27 (m, 2H), 7.18 (br s, 2H), 6.01 (br d, J = 7.5 Hz, 1H), 5.20 − 5.06 (m, 1H), 3.99 (d, J = 2.0 Hz, 3H), 2.35 (s, 3H), 1.72 (d, J = 7.0 Hz, 3H), 1.55 (d, J = 6.8 Hz, 3H). LCMS (ESI): m/z = 508.4, [M + H]⁺. |
| | N-(1-(3-methyl-4-(((R)-1-(2- | | |
| | (1-methyl-1H-pyrazol-4- | | |
| | yl)quinolin-4- | | |
| | yl)ethyl)carbamoyl)phenyl)e | | |
| | thyl)-1H-imidazole-2- | | |
| | carboxamide | | |

### Example 216: 5-(4-(1-(2-methyl-5-(4-methylpiperazin-1-yl)benzamido)ethyl)naphthalen-2-yl)-1H-pyrrole-3-carboxylic acid

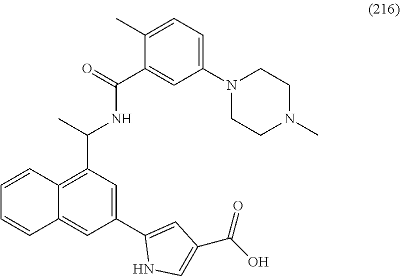 [SMILES: Cc1ccc(N2CCN(C)CC2)cc1C(=O)NC(C)c1cc(-c2cc(C(=O)O)c[nH]2)cc2ccccc12]

5-(4-(1-(2-methyl-5-(4-methylpiperazin-1-yl)benzamido)ethyl)naphthalen-2-yl)-1H-pyrrole-3-carboxylic acid was prepared from methyl 5-(4-(1-(2-methyl-5-(4-methylpiperazin-1-yl)benzamido)ethyl)naphthalen-2-yl)-1H-pyrrole-3-carboxylate using a procedure analogous to Example 71 Step 1. ¹H NMR (400 MHz, DMSO-d₆) δ=12.09 (br s, 1H), 8.87 (d, J=8.1 Hz, 1H), 8.18 (br dd, J=3.1, 6.4 Hz, 1H), 8.09 (s, 1H), 8.01 (s, 1H), 7.88 (dd, J=2.9, 6.8 Hz, 1H), 7.56-7.49 (m, 3H), 7.06 (d, J=8.4 Hz, 1H), 6.99 (s, 1H), 6.92-6.85 (m, 2H), 5.95-5.87 (m, 1H), 3.13-3.06 (m, 4H), 2.43 (br t, J=4.5 Hz, 4H), 2.19 (d, J=13.6 Hz, 6H), 1.59 (d, J=6.8 Hz, 3H). LCMS (ESI): m/z=497.3, [M+H]⁺.

### Example 217: 5-(aminomethyl)-N-(1-(4-hydroxy-3-(1-methyl-1H-pyrazol-4-yl)naphthalen-1-yl)ethyl)-2-methylbenzamide

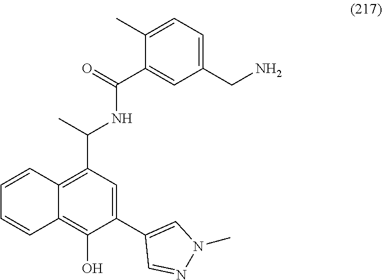 [SMILES: Cc1ccc(CN)cc1C(=O)NC(C)c1cc(-c2cnn(C)c2)c(O)c2ccccc12]

### Step 1: Preparation of tert-butyl(3-((1-(4-methoxy-3-(1-methyl-1H-pyrazol-4-yl)naphthalen-1-yl)ethyl)carbamoyl)-4-methylbenzyl)carbamate

Tert-butyl(3-((1-(4-methoxy-3-(1-methyl-1H-pyrazol-4-yl)naphthalen-1-yl)ethyl)carbamoyl)-4-methylbenzyl)carbamate was prepared using a method analogous to Example 4, Step 1, using 5-(((tert-butoxycarbonyl)amino)methyl)-2-methylbenzoic acid and 1-(4-methoxy-3-(1-methyl-1H-pyrazol-4-yl)naphthalen-1-yl)ethan-1-amine.

### Step 2: 5-(aminomethyl)-N-(1-(4-hydroxy-3-(1-methyl-1H-pyrazol-4-yl)naphthalen-1-yl)ethyl)-2-methylbenzamide

To a solution of tert-butyl(3-((1-(4-methoxy-3-(1-methyl-1H-pyrazol-4-yl)naphthalen-1-yl)ethyl)carbamoyl)-4-methylbenzyl)carbamate (300.0 mg, 0.567 mmol) in CH₂Cl₂ (30 mL) under N2 was added BBr3 (711 mg, 2.84 mmol) at 0° C. The reaction mixture was stirred at 20° C. under N2 for 15 h then quenched with 5 mL sat. NaHCO₃ at 0° C. and evaporated in vacuo to afford a crude. The crude was suspended in 8 mL MeOH, basified with 3 mL ammonia solution at 0° C., and evaporated again in vacuo to give a residue, which was dissolved in 5 mL DMF and purified by prep-HPLC. After two cycles of prep-HPLC, the 5-(aminomethyl)-N-(1-(4-hydroxy-3-(1-methyl-1H-pyrazol-4-yl)naphthalen-1-yl)ethyl)-2-methylbenzamide was obtained (51 mg, 22% yield) as a yellow solid. ¹H NMR (400 MHz, MeOD-d₄) δ ppm 8.31 (d, J=7.0 Hz, 1H), 8.25-8.14 (m, 2H), 8.09-7.94 (m, 1H), 7.79 (s, 1H), 7.59-7.48 (m, 2H), 7.38-7.25 (m, 2H), 7.25-7.16 (m, 1H), 6.03 (q, J=6.8 Hz, 1H), 4.03-3.93 (m, 3H), 3.84-3.74 (m, 2H), 2.35 (s, 3H), 1.76 (d, J=7.0 Hz, 3H). LCMS (ESI): m/z=415, [M+H]⁺.

### First Prep-HPLC Condition:

Column: Boston Prime C18 150*30 mm*5 μm;
Mobile phase: water (ammonium hydroxide)-MeCN; B %: 9%-49%,
Flow Rate (mL/min): 30 mL/min.

### Second Prep-HPLC Condition:

Column: Boston Prime C18 150*30 mm*5 μm;
Mobile phase: water (ammonium hydroxide)-MeCN; B %: 9%-45%,
Flow Rate (mL/min): 30 mL/min.

### Example 218: 2-methyl-N-(1-(2-(1-methyl-1H-pyrazol-4-yl)quinolin-4-yl)cyclopropyl)-5-(5-methylhexahydropyrrolo[3,4-c]pyrrol-2(1H)-yl)benzamide

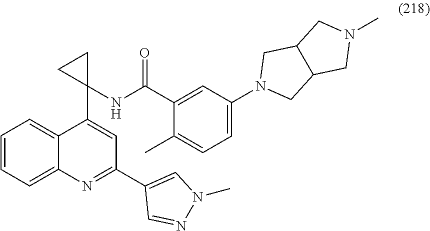 [SMILES: Cc1ccc(N2CC3CN(C)CC3C2)cc1C(=O)NC1(c2cc(-c3cnn(C)c3)nc3ccccc23)CC1]

To a solution of 5-(hexahydropyrrolo[3,4-c]pyrrol-2(1H)-yl)-2-methyl-N-(1-(2-(1-methyl-1H-pyrazol-4-yl)quinolin-4-yl)cyclopropyl)benzamide (250.0 mg, 0.507 mmol) in MeOH (10 mL) were added HCHO (0.5 mL, 38%), HOAc (0.3 mL), and then NaBH₃CN (63.8 mg, 1.01 mmol) at 20° C. The reaction mixture was stirred at 20° C. for 3 h, then partitioned between ethyl acetate and aq. NaHCO₃ (100/50 mL). The organic layer was separated, and the aqueous layer was re-extracted with ethyl acetate (50 mL). The combined organic layer was washed with brine, dried with anhydrous Na₂SO₄ and evaporated in vacuo to afford a crude, which was then dissolved in 5 mL DMF and purified by prep-HPLC to afford 2-methyl-N-(1-(2-(1-methyl-1H-pyrazol-4-yl)quinolin-4-yl)cyclopropyl)-5-(5-methylhexahydropyrrolo[3,4-c]pyrrol-2(1H)-yl)benzamide (57 mg, 20%) as a white solid after lyophilization. ¹H NMR (400 MHz, MeOD-d₄) δ ppm 8.56 (d, J=8.4 Hz, 1H), 8.49 (s, 1H), 8.41 (s, 1H), 8.27 (s, 1H), 8.10 (s, 1H), 8.07 (d, J=8.6 Hz, 1H), 7.76 (t, J=7.3 Hz, 1H), 7.61 (t, J=7.6 Hz, 1H), 7.01 (d, J=8.4 Hz, 1H), 6.73 (d, J=8.6 Hz, 1H), 6.50 (d, J=2.6 Hz, 1H), 4.03 (s, 3H), 3.58-3.44 (m, 2H), 3.41-3.35 (m, 2H), 3.23-3.02 (m, 6H), 2.81 (s, 3H), 2.01 (s, 3H), 1.54-1.47 (m, 2H), 1.47-1.41 (m, 2H). LCMS (ESI): m/z=507.5, [M+H]⁺.

### Prep-HPLC Condition:

Column: Boston Prime C18 150*30 mm*5 μm;
Mobile phase: water (FA)-MeCN; B %: 0%-30%,
Flow Rate (mL/min): 30 mL/min.

### Examples 249, 256, 262 and 266

The following examples were prepared in a similar manner to Example 218 using an appropriate amine and formaldehyde.

| Ex. | Structure; | Amine, reducing | |
|---|---|---|---|
| # | Compound Name | agent, solvent | Characterizing Data |
| Ex. 249 | 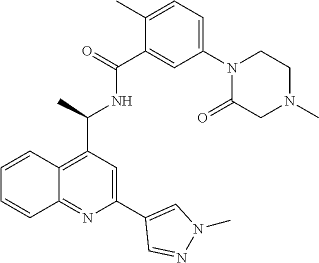 [SMILES: Cc1ccc(N2CCN(C)CC2=O)cc1C(=O)N[C@H](C)c1cc(-c2cnn(C)c2)nc2ccccc12] | (R)-2-methyl-N-(1- (2-(1-methyl-1H- pyrazol-4- yl)quinolin-4- yl)ethyl)-5-(2- oxopiperazin-1- yl)benzamide, NaBH(OAc)₃, THF | ¹H NMR (400 MHz, DMSO-d₆) δ = 9.00 (br d, J = 7.5 Hz, 1H), 8.42 (s, 1H), 8.21 (br d, J = 8.4 Hz, 1H), 8.09 (s, 1H), 7.97 (br d, J = 8.3 Hz, 1H), 7.84 (s, 1H), 7.73 (br t, J = 7.5 Hz, 1H), 7.57 (brt, J = 7.3 Hz, 1H), 7.38 (s, 1H), 7.32 − 7.24 (m, 2H), 5.87 (br t, J = 7.1 Hz, 1H), 3.94 (s, 3H), 3.65 (br t, J = 5.1 Hz, 2H), 3.12 (s, 2H), 2.77 − 2.67 (m, 1H), 2.85 − 2.63 (m, 1H), 2.28 (br d, J = 4.5 Hz, 6H), 1.61 (br d, J = 6.8 Hz, 3H). LCMS (ESI): m/z = 483.3, [M + H]⁺. |
| | (R)-2-methyl-N-(1-(2-(1- | | |
| | methyl-1H-pyrazol-4- | | |
| | yl)quinolin-4-yl)ethyl)-5-(4- | | |
| | methyl-2-oxopiperazin-1- | | |
| | yl)benzamide | | |
| Ex. 256 | 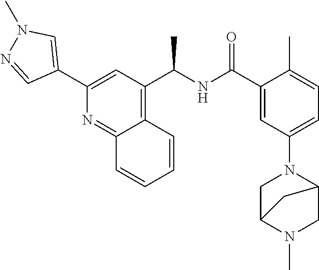 [SMILES: Cc1ccc(N2CC3CC2CN3C)cc1C(=O)N[C@H](C)c1cc(-c2cnn(C)c2)nc2ccccc12] | 5-(2,5- diazabicyclo[2.2.1] heptan-2-yl)-2- methyl-N-((R)-1-(2- (1-methyl-1H- pyrazol-4- yl)quinolin-4- yl)ethyl)benzamide, NaBH3CN/HOAc, DCM | ¹H NMR (400 MHz, MeOD-d₄) δ = 8.48 (s, 1H), 8.35 (s, 1H), 8.29 (d, J = 8.1 Hz, 1H), 8.17 (s, 1H), 8.08 (d, J = 8.6 Hz, 1H), 7.88 (s, 1H), 7.79 (t, J = 7.2 Hz, 1H), 7.66 − 7.61 (m, 1H), 7.15 (d, J = 8.6 Hz, 1H), 6.71 (d, J = 8.4 Hz, 1H), 6.63 (d, J = 2.0 Hz, 1H), 6.04 (d, J = 7.0 Hz, 1H), 4.27 (s, 1H), 4.02 (s, 3H), 3.73 − 3.67 (m, 1H), 3.56 − 3.48 (m, 1H), 3.39 (s, 2H), 3.26 − 3.16 (m, 1H), 2.87 (s, 3H), 2.35 − 2.27 (m, 2H), 2.26 (s, 3H), 1.75 (d, J = 6.8 Hz, 3H). LCMS (ESI): m/z = 481.4 [M + H]⁺. |
| | 2-methyl-N-((R)-1-(2-(1- | | |
| | methyl-1H-pyrazol-4- | | |
| | yl)quinolin-4-yl)ethyl)-5-(5- | | |
| | methyl-2,5- | | |
| | diazabicyclo[2.2.1]heptan- | | |
| | 2-yl)benzamide | | |
| Ex. 262 | 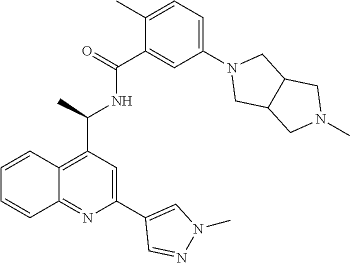 [SMILES: Cc1ccc(N2CC3CN(C)CC3C2)cc1C(=O)N[C@H](C)c1cc(-c2cnn(C)c2)nc2ccccc12] | 5-(hexahydropyrrolo [3,4-c]pyrrol-2(1H)- yl)-2-methyl-N-((R)- 1-(2-(1-methyl-1H- pyrazol-4- yl)quinolin-4-yl) ethyl)benzamide, NaBH3CN/HOAc, MeOH | ¹H NMR (400 MHz, MeOD-d₄) · ppm 1.74 (d, J = 7.2 Hz, 3H), 2.25 (s, 3H), 2.37 (s, 3H), 2.43 − 2.45 (m, 2H), 2.89 − 2.96 (m, 2H), 3.00 − 3.01 (m, 2H), 3.20 − 3.29 (m, 4H), 4.02 (s, 3H), 5.96 − 6.10 (m, 1H), 6.64 − 6.78 (m, 2H), 7.08 (d, J = 8.31 Hz, 1H), 7.57 − 7.70 (m, 1H), 7.78 (td, J = 7.67, 1.04 Hz, 1H), 7.86 − 7.92 (m, 1H), 8.02 − 8.10 (m, 1H), 8.12 − 8.21 (m, 1H), 8.22 − 8.40 (m, 2H). LCMS (ESI): m/z = 495.4 [M + H]⁺. |
| | 2-methyl-N-((R)-1-(2-(1- | | |
| | methyl-1H-pyrazol-4- | | |
| | yl)quinolin-4-yl)ethyl)-5-(5- | | |
| | methylhexahydropyrrolo | | |
| | [3,4-c]pyrrol-2(1H)- | | |
| | yl)benzamide | | |
| Ex. 266 | 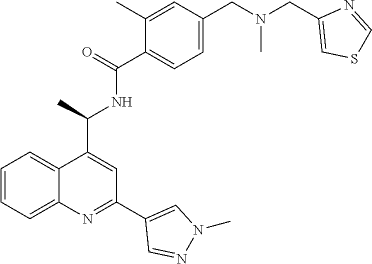 [SMILES: Cc1cc(CN(C)Cc2cscn2)ccc1C(=O)N[C@H](C)c1cc(-c2cnn(C)c2)nc2ccccc12] | (R)-2-methyl-N-(1- (2-(1-methyl-1H- pyrazol-4- yl)quinolin-4- yl)ethyl)-4-(((thiazol- 4-ylmethyl)amino) methyl)benzamide, NaBH3CN/HOAc, MeOH | ¹H NMR (400 MHz, MeOD-d₄) δ = 9.00 (d, J = 1.8 Hz, 1H), 8.34 (s, 1H), 8.29 (d, J = 8.3 Hz, 1H), 8.18 (s, 1H), 8.08 (d, J = 8.3 Hz, 1H), 7.87 (s, 1H), 7.78 (t, J = 7.5 Hz, 1H), 7.63 (t, J = 7.7 Hz, 1H), 7.50 (d, J = 1.7 Hz, 1H), 7.39 − 7.34 (m, 1H), 7.31 − 7.27 (m, 2H), 6.04 (q, J = 7.2 Hz, 1H), 4.02 (s, 3H), 3.78 (s, 2H), 3.59 (s, 2H), 2.37 (s, 3H), 2.24 (s, 3H), 1.74 (d, J = 7.0 Hz, 3H). LCMS (ESI): m/z = 511.4, [M + H]⁺. |
| | (R)-2-methyl-4-((methyl | | |
| | (thiazol-4-ylmethyl)amino) | | |
| | methyl)-N-(1-(2-(1-methyl- | | |
| | 1H-pyrazol-4-yl)quinolin-4- | | |
| | yl)ethyl)benzamide | | |

### Example 219: (2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylic acid

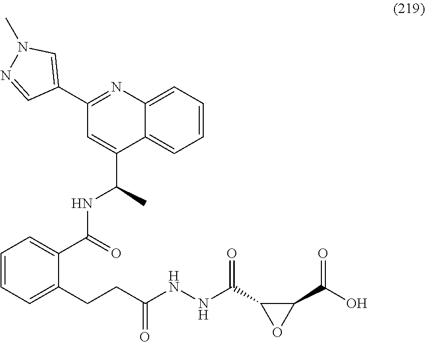 [SMILES: C[C@@H](NC(=O)c1ccccc1CCC(=O)NNC(=O)[C@H]1O[C@@H]1C(=O)O)c1cc(-c2cnn(C)c2)nc2ccccc12]

To a solution of ethyl(2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylate (20.0 mg, 0.0342 mmol) in THF (1.0 mL) and H2O (0.3 mL) was added LiOH·H₂O (2.87 mg, 0.0684 mmol) at 20° C. The mixture was stirred at 20° C. for 0.5 h then evaporated in vacuo to give a crude, which was then purified by prep-HPLC to afford (2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylic acid (12.57 mg, 66.0%) as a white solid.

¹H NMR (400 MHz, DMSO-d₆) δ=10.40 (s, 1H), 10.02 (s, 1H), 9.02 (d, J=7.8 Hz, 1H), 8.44 (s, 1H), 8.23 (d, J=8.1 Hz, 1H), 8.12 (s, 1H), 7.98 (d, J=7.6 Hz, 1H), 7.87 (s, 1H), 7.74 (t, J=7.6 Hz, 1H), 7.59 (t, J=7.6 Hz, 1H), 7.40-7.26 (m, 4H), 5.91 (t, J=7.2 Hz, 1H), 3.94 (s, 3H), 3.64 (d, J=1.7 Hz, 1H), 3.50 (d, J=1.7 Hz, 1H), 2.94 (br t, J=7.8 Hz, 2H), 2.49-2.41 (m, 2H), 1.63 (d, J=6.8 Hz, 3H). LCMS (ESI): m/z=557.4, [M+H]⁺.

### Prep-HPLC Condition:

Column: Welch Xtimate C18 150*25 mm*5 μm;
Mobile phase: water (FA)-MeCN; B %: 10%-30%,
Flow Rate (mL/min): 60 mL/min.

### Example 220: methyl(2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylate

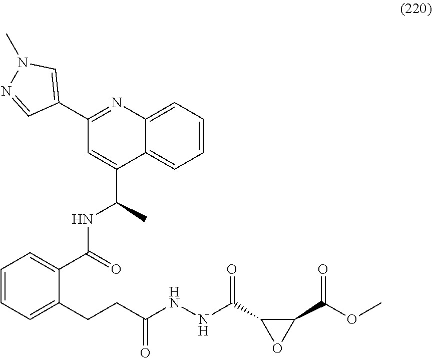 [SMILES: COC(=O)[C@H]1O[C@@H]1C(=O)NNC(=O)CCc1ccccc1C(=O)N[C@H](C)c1cc(-c2cnn(C)c2)nc2ccccc12]

To a solution of ethyl(2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylate (40.0 mg, 0.0684 mmol) in MeOH (3.0 mL), THF (3.0 mL) and H₂O (1.0 mL) was added LiOH·H₂O (5.74 mg, 0.137 mmol) at 20° C. The mixture was stirred at 20° C. for 0.5 h at which time, LC-MS showed the starting material was almost consumed and a desired product was formed. The mixture was then acidified with aq. HCl(1 N) to pH=5-6 followed by evaporation in vacuo and purified by prep-HPLC to afford methyl(2S,3S)-3-(2-(3-(2-(((R)-1-(2-(1-methyl-1H-pyrazol-4-yl)quinolin-4-yl)ethyl)carbamoyl)phenyl)propanoyl)hydrazine-1-carbonyl)oxirane-2-carboxylate (15.42 mg, 39.2, FA salt) as a white solid. ¹H NMR (400 MHz, DMSO-d₆) δ=10.46 (br s, 1H), 10.05 (s, 1H), 9.02 (d, J=7.8 Hz, 1H), 8.43 (s, 1H), 8.23 (d, J=8.1 Hz, 1H), 8.15-8.09 (m, 1H), 7.98 (d, J=8.5 Hz, 1H), 7.87 (s, 1H), 7.74 (t, J=7.3 Hz, 1H), 7.62-7.55 (m, 1H), 7.40-7.26 (m, 4H), 5.91 (quin, J=7.1 Hz, 1H), 3.94 (s, 3H), 3.76-3.67 (m, 5H), 2.94 (br t, J=7.8 Hz, 2H), 2.49-2.44 (m, 2H), 1.63 (d, J=6.8 Hz, 3H). LCMS (ESI): m/z=571.3, [M+H]⁺. ee %=100% by SFC.

### Prep-HPLC Condition:

Column: Boston Prime C18 150*30 mm*5 μm;
Mobile phase: water (FA)-MeCN; B %: 10%-30%,
Flow Rate (mL/min): 35 mL/min.

### Example 221: (R)-2-methyl-4-((((1-methyl-1H-imidazol-5-yl)methyl)amino)methyl)-N-(1-(2-(1-methyl-1H-pyrazol-4-yl)quinolin-4-yl)ethyl)benzamide

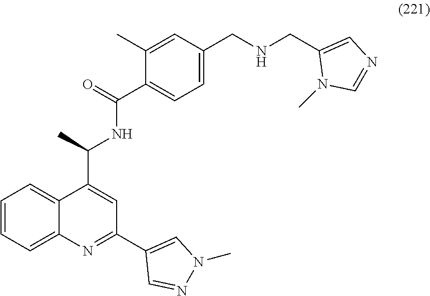 [SMILES: Cc1cc(CNCc2cncn2C)ccc1C(=O)N[C@H](C)c1cc(-c2cnn(C)c2)nc2ccccc12]

(R)-2-methyl-4-((((1-methyl-1H-imidazol-5-yl)methyl)amino)methyl)-N-(1-(2-(1-methyl-1H-pyrazol-4-yl)quinolin-4-yl)ethyl)benzamide was prepared in a similar manner to Example 6 using 1-methyl-1H-imidazole-5-carbaldehyde and (R)-4-(aminomethyl)-2-methyl-N-(1-(2-(1-methyl-1H-pyrazol-4-yl)quinolin-4-yl)ethyl)benzamide as the appropriate amine and aldehyde. ¹H NMR (400 MHz, MeOD-d₄) δ ppm 8.35 (s, 1H), 8.29 (d, J=7.8 Hz, 1H), 8.18 (s, 1H), 8.08 (d, J=8.4 Hz, 1H), 7.87 (s, 1H), 7.78 (t, J=7.1 Hz, 1H), 7.63 (t, J=7.7 Hz, 1H), 7.37 (d, J=7.1 Hz, 1H), 7.29-7.22 (m, 2H), 7.02 (s, 1H), 6.88 (s, 1H), 6.08-6.01 (m, 1H), 4.02 (s, 3H), 3.82 (s, 2H), 3.78 (s, 2H), 3.69 (s, 3H), 2.37 (s, 3H), 1.75 (d, J=7.0 Hz, 3H). LCMS (ESI): m/z=494.4, [M+H]⁺.

### Examples 222-227, 232-234 and 250

The following examples were prepared in a similar manner to Example 63 using appropriate reactants.

| Ex. | Structure; | | |
|---|---|---|---|
| # | Compound Name | Reactants used | Characterizing Data |
| Ex. 222 | 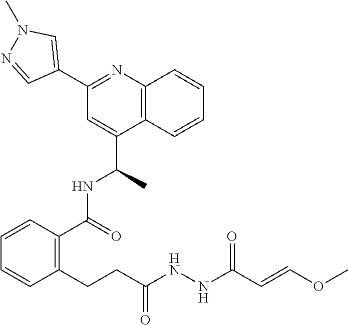 [SMILES: CO/C=C/C(=O)NNC(=O)CCc1ccccc1C(=O)N[C@H](C)c1cc(-c2cnn(C)c2)nc2ccccc12] | Step 4: (R)-2-(3- hydrazineyl-3- oxopropyl)-N-(1- (2-(1-methyl-1H- pyrazol-4- yl)quinolin-4- yl)ethyl) benzamide, (E)-3- methoxyacrylic acid and Mukaiyama′ reagent instead of HATU. | ¹H NMR (400 MHz, DMSO-d₆) δ = 9.79 (br s, 1H), 9.57 (br s, 1H), 9.10 − 8.98 (m, 1H), 8.45 (s, 1H), 8.25 (br d, J = 8.3 Hz, 1H), 8.13 (s, 1H), 7.99 (br d, J = 8.4 Hz, 1H), 7.89 (s, 1H), 7.75 (br t, J = 7.6 Hz, 1H), 7.60 (br t, J = 7.6 Hz, 1H), 7.52 − 7.26 (m, 5H), 5.97 − 5.87 (m, 1H), 5.40 (d, J = 12.5 Hz, 1H), 3.95 (s, 3H), 3.74 − 3.47 (m, 2H), 2.95 (br t, J = 7.6 Hz, 2H), 2.49 − 2.35 (m, 2H), 1.65 (br d, J = 6.8 Hz, 3H). LCMS (ESI): m/z = 527.3, [M + H]⁺. |
| | (R,E)-2-(3-(2-(3- | | |
| | methoxyacryloyl) | | |
| | hydrazineyl)-3-oxopropyl)- | | |
| | N-(1-(2-(1-methyl-1H- | | |
| | pyrazol-4-yl)quinolin-4- | | |
| | yl)ethyl)benzamide | | |
| Ex. 223 | 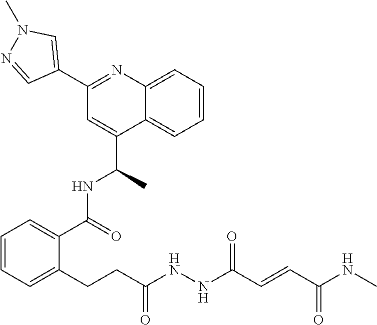 [SMILES: CNC(=O)/C=C/C(=O)NNC(=O)CCc1ccccc1C(=O)N[C@H](C)c1cc(-c2cnn(C)c2)nc2ccccc12] | Step 4: (R)-2-(3- hydrazineyl-3- oxopropyl)-N-(1- (2-(1-methyl-1H- pyrazol-4- yl)quinolin-4- yl)ethyl) benzamide, (E)-4- (methylamino)-4- oxobut-2-enoic acid with N-Ethyl- N′-(3-dimethyl aminopropyl) carbodiimide and pyridine instead of HATU and DIEA. | ¹H NMR (400 MHz, DMSO-d₆) δ = 9.02 (br d, J = 7.5 Hz, 1H), 8.46 − 8.38 (m, 2H), 8.25 (d, J = 8.3 Hz, 1H), 8.13 (s, 1H), 7.99 (d, J = 8.4 Hz, 1H), 7.88 (s, 1H), 7.75 (t, J = 7.8 Hz, 1H), 7.60 (t, J = 7.2 Hz, 1H), 7.41 − 7.28 (m, 4H), 6.89 (d, J = 3.5 Hz, 2H), 5.92 (br t, J = 7.3 Hz, 1H), 3.95 (s, 3H), 2.96 (br t, J = 7.8 Hz, 2H), 2.71 (d, J = 4.6 Hz, 3H), 2.50 − 2.45 (m, 2H), 1.65 (d, J = 6.8 Hz, 3H). LCMS (ESI): m/z = 554.4, [M + H]⁺. |
| | (R,E)-N-(1-(2-(1-methyl-1H- | | |
| | pyrazol-4-yl)quinolin-4- | | |
| | yl)ethyl)-2-(3-(2-(4- | | |
| | (methylamino)-4-oxobut-2- | | |
| | enoyl)hydrazineyl)-3- | | |
| | oxopropyl)benzamide | | |
| Ex. 224 | 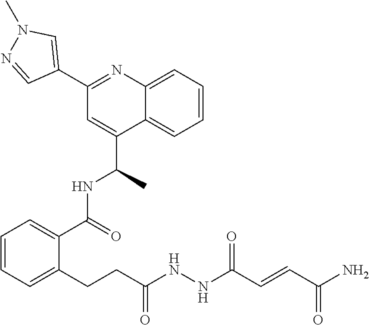 [SMILES: C[C@@H](NC(=O)c1ccccc1CCC(=O)NNC(=O)/C=C/C(N)=O)c1cc(-c2cnn(C)c2)nc2ccccc12] | Step 4: (R)-2-(3- hydrazineyl-3- oxopropyl)-N-(1- (2-(1-methyl-1H- pyrazol-4-yl) quinolin-4-yl)ethyl) benzamide, (E)-4- amino-4-oxobut-2- enoic acid with N- Ethyl-N′-(3- dimethylaminopro pyl)carbodiimide and pyridine instead of HATU and DIEA. | ¹H NMR (400 MHz, DMSO-d₆) δ = 9.91 (br s, 1H), 9.04 (br d, J = 7.6 Hz, 1H), 8.46 (s, 1H), 8.25 (br d, J = 7.9 Hz, 1H), 8.14 (s, 1H), 7.99 (br d, J = 8.2 Hz, 1H), 7.93 − 7.81 (m, 2H), 7.75 (br t, J = 7.3 Hz, 1H), 7.61 (br t, J = 7.3 Hz, 1H), 7.47 − 7.22 (m, 6H), 6.97 − 6.78 (m, 2H), 5.92 (br d, J = 7.1 Hz, 1H), 3.96 (s, 3H), 2.96 (br d, J = 7.0 Hz, 3H), 2.49 − 2.38 (m, 2H), 1.65 (br d, J = 6.6 Hz, 3H). LCMS (ESI): m/z = 540.3, [M + H]⁺. |
| | (R,E)-2-(3-(2-(4-amino-4- | | |
| | oxobut-2-enoyl)hydrazineyl)- | | |
| | 3-oxopropyl)-N-(1-(2-(1- | | |
| | methyl-1H-pyrazol-4- | | |
| | yl)quinolin-4-yl)ethyl) | | |
| | benzamide | | |
| Ex. 225 | 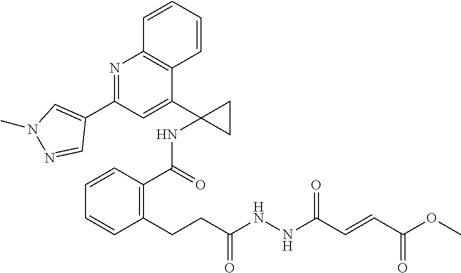 | Step 4: 2-(3- hydrazineyl-3- oxopropyl)-N-(1- (2-(1-methyl-1H- pyrazol-4-yl) quinolin-4-yl) cyclopropyl) benzamide and (E)-4-methoxy-4- oxobut-2-enoic acid. | ¹H NMR (400 MHz, MeOD-d₄) δ ppm 8.57 (d, J = 7.9 Hz, 1H), 8.42 (s, 1H), 8.27 (s, 1H), 8.11 (s, 1H), 8.07 (d, J = 7.8 Hz, 1H), 7.76 (t, J = 7.1 Hz, 1H), 7.63 (t, J = 7.2 Hz, 1H), 7.36 − 7.24 (m, 2H), 7.23 − 7.10 (m, 2H), 7.04 (d, J = 15.5 Hz, 1H), 6.83 (d, J = 15.6 Hz, 1H), 4.02 (s, 3H), 3.82 (s, 3H), 2.90 − 2.78 (m, 2H), 2.43 (t, J = 7.5 Hz, 2H), 1.58 − 1.38 (m, 4H). LCMS (ESI): m/z = 567.3, [M + H]. |
| | methyl (E)-4-(2-(3-(2-((1-(2- | | |
| | (1-methyl-1H-pyrazol-4- | | |
| | yl)quinolin-4-yl)cyclopropyl) | | |
| | carbamoyl)phenyl) | | |
| | propanoyl)hydrazineyl)-4- | | |
| | oxobut-2-enoate | | |
| Ex. 226 | 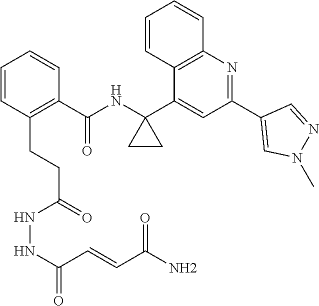 [SMILES: Cn1cc(-c2cc(C3(NC(=O)c4ccccc4CCC(=O)NNC(=O)/C=C/C(N)=O)CC3)c3ccccc3n2)cn1] | Step 4: 2-(3- hydrazineyl-3- oxopropyl)-N-(1- (2-(1-methyl-1H- pyrazol-4- yl)quinolin-4- yl)cyclopropyl) benzamide, (E)-4- amino-4-oxobut-2- enoic acid with N- Ethyl-N′-(3- dimethylaminopro pyl)carbodiimide and pyridine instead of HATU and DIEA. | ¹H NMR (400 MHz, MeOD-d₄) δ ppm 10.37 (br s, 1H), 10.04 (br s, 1H), 9.37 (s, 1H), 8.60 − 8.54 (m, 2H), 8.23 − 8.17 (m, 1H), 7.96 (s, 1H), 7.97 (d, J = 6.5 Hz, 1H), 7.85 (br s, 1H), 7.75 − 7.68 (m, 1H), 7.57 (t, J = 7.6 Hz, 1H), 7.41 (br s, 1H), 7.34 − 7.22 (m, 2H), 7.17 (t, J = 6.8 Hz, 1H), 7.03 − 6.83 (m, 3H), 3.94 (s, 3H), 2.81 (br t, J = 7.8 Hz, 2H), 2.43 − 2.31 (m, 2H), 1.41 (br s, 2H), 1.38 − 1.31 (m, 2H). LCMS (ESI): m/z = 552, [M + H]⁺. |
| | (E)-2-(3-(2-(4-amino-4- | | |
| | oxobut-2-enoyl)hydrazineyl)- | | |
| | 3-oxopropyl)-N-(1-(2-(1- | | |
| | methyl-1H-pyrazol-4- | | |
| | yl)quinolin-4-yl)cyclopropyl) | | |
| | benzamide | | |
| Ex. 227 | 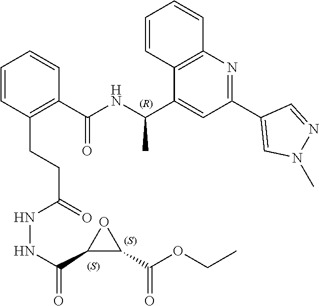 | Step 4: (R)-2-(3- hydrazineyl-3- oxopropyl)-N-(1- (2-(1-methyl-1H- pyrazol-4- yl)quinolin-4- yl)ethyl) benzamide and (2S,3S)-3- (ethoxycarbonyl) oxirane-2- carboxylic acid. | ¹H NMR (400 MHz, DMSO-d₆) δ = 10.46 (d, J = 1.6 Hz, 1H), 10.05 (d, J = 1.8 Hz, 1H), 9.03 (d, J = 7.9 Hz, 1H), 8.46 (s, 1H), 8.25 (br d, J = 7.9 Hz, 1H), 8.14 (s, 1H), 7.99 (br d, J = 8.4 Hz, 1H), 7.89 (s, 1H), 7.76 (br t, J = 7.6 Hz, 1H), 7.60 (br t, J = 7.3 Hz, 1H), 7.40 − 7.26 (m, 4H), 5.91 (br t, J = 7.3 Hz, 1H), 4.20 (q, J = 7.0 Hz, 2H), 3.94 (s, 3H), 3.73 − 3.64 (m, 2H), 2.93 (br t, J = 7.7 Hz, 2H), 2.49 − 2.43 (m, 2H), 1.63 (d, J = 6.8 Hz, 3H), 1.28 − 1.16 (m, 3H). LCMS (ESI): m/z = 585.3, [M + H]⁺. ee % > 95% by SFC. |
| | ethyl (2S,3S)-3-(2-(3-(2- | | |
| | (((R)-1-(2-(1-methyl-1H- | | |
| | pyrazol-4-yl)quinolin-4- | | |
| | yl)ethyl)carbamoyl)phenyl) | | |
| | propanoyl)hydrazine-1- | | |
| | carbonyl)oxirane-2- | | |
| | carboxylate | | |
| Ex. 232 | 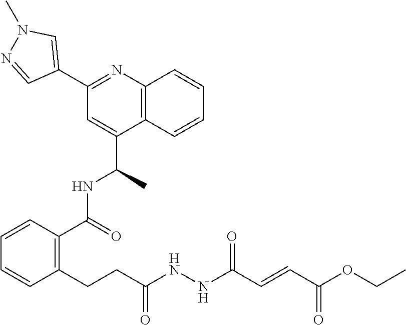 [SMILES: CCOC(=O)/C=C/C(=O)NNC(=O)CCc1ccccc1C(=O)N[C@H](C)c1cc(-c2cnn(C)c2)nc2ccccc12] | Step 4: (R)-2-(3- hydrazineyl-3- oxopropyl)-N-(1- (2-(1-methyl-1H- pyrazol-4- yl)quinolin-4- yl)ethyl) benzamide and (E)-4-ethoxy-4- oxobut-2-enoic acid. | ¹H NMR (400 MHz, DMSO-d₆) δ = 10.25 (br s, 2H), 9.03 (br d, J = 7.8 Hz, 1H), 8.44 (s, 1H), 8.24 (d, J = 8.5 Hz, 1H), 8.12 (s, 1H), 7.98 (d, J = 8.4 Hz, 1H), 7.88 (s, 1H), 7.74 (t, J = 7.6 Hz, 1H), 7.59 (t, J = 7.5 Hz, 1H), 7.42 − 7.25 (m, 4H), 7.07 (d, J = 15.6 Hz, 1H), 6.66 (d, J = 15.5 Hz, 1H), 5.91 (quin, J = 6.9 Hz, 1H), 4.26 − 4.11 (m, 2H), 3.94 (s, 3H), 3.01 − 2.87 (m, 2H), 2.53 (br s, 2H), 1.64 (br d, J = 6.9 Hz, 3H), 1.31 − 1.18 (m, 3H). LCMS (ESI): m/z = 569.3, [M + H]⁺. |
| | ethyl (R,E)-4-(2-(3-(2-((1-(2- | | |
| | (1-methyl-1H-pyrazol-4- | | |
| | yl)quinolin-4-yl)ethyl) | | |
| | carbamoyl)phenyl) | | |
| | propanoyl)hydrazineyl)-4- | | |
| | oxobut-2-enoate | | |
| Ex. 233 | 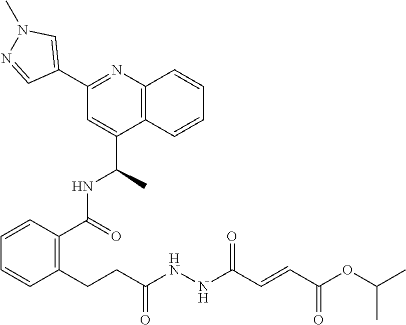 [SMILES: CC(C)OC(=O)/C=C/C(=O)NNC(=O)CCc1ccccc1C(=O)N[C@H](C)c1cc(-c2cnn(C)c2)nc2ccccc12] | Step 4: (R)-2-(3- hydrazineyl-3- oxopropyl)-N-(1- (2-(1-methyl-1H- pyrazol-4- yl)quinolin-4- yl)ethyl) benzamide and (E)-4-isopropoxy- 4-oxobut-2-enoic acid. | ¹H NMR (400 MHz, DMSO-d₆) δ = 10.27 (br s, 1H), 9.03 (br d, J = 7.3 Hz, 1H), 8.45 (s, 1H), 8.25 (br d, J = 8.1 Hz, 1H), 8.13 (s, 1H), 7.99 (br d, J = 8.3 Hz, 1H), 7.89 (s, 1H), 7.75 (br t, J = 7.5 Hz, 1H), 7.60 (br t, J = 7.3 Hz, 1H), 7.40 − 7.28 (m, 4H), 7.06 (d, J = 15.5 Hz, 1H), 6.64 (d, J = 15.6 Hz, 1H), 5.93 (br s, 1H), 5.02 (br d, J = 6.1 Hz, 1H), 3.95 (s, 3H), 2.96 (br t, J = 7.8 Hz, 2H), 2.50 − 2.43 (m, 2H), 1.65 (br d, J = 6.7 Hz, 3H), 1.27 (br d, J = 6.1 Hz, 6H). LCMS (ESI): m/z = 583.3, [M + H]⁺. ee % = 100% by SFC. |
| | isopropyl (R,E)-4-(2-(3-(2- | | |
| | ((1-(2-(1-methyl-1H-pyrazol- | | |
| | 4-yl)quinolin-4- | | |
| | yl)ethyl)carbamoyl)phenyl) | | |
| | propanoyl)hydrazineyl)-4- | | |
| | oxobut-2-enoate | | |
| Ex. 234 | 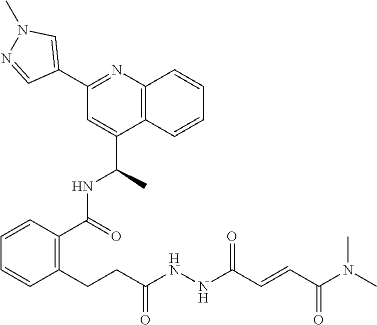 [SMILES: C[C@@H](NC(=O)c1ccccc1CCC(=O)NNC(=O)/C=C/C(=O)N(C)C)c1cc(-c2cnn(C)c2)nc2ccccc12] | Step 4: (R)-2-(3- hydrazineyl-3- oxopropyl)-N-(1- (2-(1-methyl-1H- pyrazol-4- yl)quinolin-4- yl)ethyl) benzamide and (E)-4-(dimethyl amino)-4-oxobut- 2-enoic acid with N-Ethyl-N′-(3- dimethylaminopro pyl)carbodiimide and pyridine instead of HATU and DIEA. | ¹H NMR (400 MHz, DMSO-d₆) δ = 10.36 (br s, 1H), 10.07 (br s, 1H), 9.02 (br d, J = 7.6 Hz, 1H), 8.44 (s, 1H), 8.23 (d, J = 8.2 Hz, 1H), 8.12 (s, 1H), 7.98 (d, J = 8.1 Hz, 1H), 7.87 (s, 1H), 7.74 (t, J = 7.3 Hz, 1H), 7.59 (t, J = 7.6 Hz, 1H), 7.41 − 7.26 (m, 5H), 6.85 (d, J = 15.0 Hz, 1H), 5.91 (br t, J = 7.2 Hz, 1H), 3.94 (s, 3H), 3.08 (s, 3H), 2.98 − 2.88 (m, 5H), 2.49 − 2.44 (m, 2H), 1.63 (br d, J = 7.0 Hz, 3H). LCMS (ESI): m/z = 568.3, [M + H]⁺. ee % > 98% by SFC. |
| | (R,E)-2-(3-(2-(4- | | |
| | (dimethylamino)-4-oxobut- | | |
| | 2-enoyl)hydrazineyl)-3- | | |
| | oxopropyl)-N-(1-(2-(1- | | |
| | methyl-1H-pyrazol-4- | | |
| | yl)quinolin-4- | | |
| | yl)ethyl)benzamide | | |
| Ex. 250 | 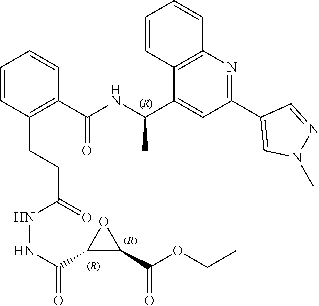 | Step 4: (R)-2-(3- hydrazineyl-3- oxopropyl)-N-(1- (2-(1-methyl-1H- pyrazol-4- yl)quinolin-4- yl)ethyl) benzamide and (2R,3R)-3- (ethoxycarbonyl) oxirane-2- carboxylic acid. | ¹H NMR (400 MHz, MeOD-d₄) δ = 8.27 − 8.15 (m, 2H), 8.11 − 8.05 (m, 1H), 7.96 (d, J = 7.9 Hz, 1H), 7.78 (s, 1H), 7.67 (t, J = 7.6 Hz, 1H), 7.53 (t, J = 7.1 Hz, 1H), 7.32 − 7.16 (m, 4H), 5.93 (q, J = 6.7 Hz, 1H), 4.15 (q, J = 7.1 Hz, 2H), 3.90 (s, 3H), 3.57 (dd, J = 1.8, 16.0 Hz, 1H), 3.00 − 2.87 (m, 2H), 2.54 − 2.42 (m, 2H), 1.64 (d, J = 7.0 Hz, 3H), 1.28 − 1.09 (m, 3H). LCMS (ESI): m/z = 585.5, [M + H]⁺. |
| | ethyl (2R,3R)-3-(2-(3-(2- | | |
| | (((R)-1-(2-(1-methyl-1H- | | |
| | pyrazol-4-yl)quinolin-4- | | |
| | yl)ethyl)carbamoyl)phenyl) | | |
| | propanoyl)hydrazine-1- | | |
| | carbonyl)oxirane-2- | | |
| | carboxylate | | |

### Example 229: N,N-dimethyl-5-(4-(1-(2-methyl-5-(4-methylpiperazin-1-yl)benzamido)ethyl) naphthalen-2-yl)-1H-pyrrole-3-carboxamide

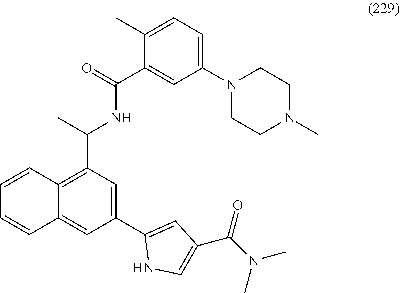 [SMILES: Cc1ccc(N2CCN(C)CC2)cc1C(=O)NC(C)c1cc(-c2cc(C(=O)N(C)C)c[nH]2)cc2ccccc12]

N,N-dimethyl-5-(4-(1-(2-methyl-5-(4-methylpiperazin-1-yl)benzamido)ethyl) naphthalen-2-yl)-1H-pyrrole-3-carboxamide was prepared in a similar manner to Example 71, Step 2 using 5-(4-(1-(2-methyl-5-(4-methylpiperazin-1-yl)benzamido)ethyl)naphthalen-2-yl)-1H-pyrrole-3-carboxylic acid and Me₂NH·HCl as the appropriate amine and carboxylic acid.

¹H NMR (400 MHz, DMSO-d₆) δ=11.88 (br s, 1H), 8.87 (d, J=8.1 Hz, 1H), 8.20-8.15 (m, 1H), 8.07 (s, 1H), 8.00 (d, J=1.5 Hz, 1H), 7.90-7.85 (m, 1H), 7.56-7.50 (m, 2H), 7.32-7.29 (m, 1H), 7.06 (d, J=8.4 Hz, 1H), 6.93-6.85 (m, 3H), 5.90 (br t, J=7.4 Hz, 1H), 3.17-3.02 (m, 9H), 2.45-2.39 (m, 4H), 2.20 (s, 3H), 2.18 (s, 3H), 1.60 (d, J=6.9 Hz, 3H).

LCMS (ESI): m/z=524.4, [M+H]⁺.

### Example 257-258

The following examples were prepared in a similar manner to Example 71 using appropriate reactants.

| Ex. | Structure; | | |
|---|---|---|---|
| # | Compound Name | Reactants used | Characterizing Data |
| Ex. 257 | 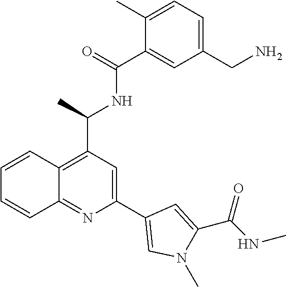 [SMILES: CNC(=O)c1cc(-c2cc([C@@H](C)NC(=O)c3cc(CN)ccc3C)c3ccccc3n2)cn1C] (R)-4-(4-(1-(5- | Step 1: methyl (R)- 4-(4-(1-(5-(((tert- butoxycarbonyl)ami no)methyl)-2- methylbenzamido) ethyl)quinolin-2-yl)- 1-methyl-1H-pyrrole- 2-carboxylate and NaOH instead of LiOH. Step 2: (R)-4-(4-(1- (5-(((tert- butoxycarbonyl)ami no)methyl)-2- methylbenzamido)et hyl)quinolin-2-yl)-1- methyl-1H-pyrrole-2- carboxylic acid and MeNH₂; | ¹H NMR (400 MHz, DMSO-d₆) δ = 8.90 (br d, J = 7.3 Hz, 1H), 8.26 (br d, J = 4.8 Hz, 1H), 8.21 (d, J = 8.4 Hz, 1H), 7.94 (d, J = 8.4 Hz, 1H), 7.85 (s, 1H), 7.74 − 7.69 (m, 2H), 7.57 − 7.52 (m, 1H), 7.46 (s, 1H), 7.36 (s, 1H), 7.28 (br d, J = 7.7 Hz, 1H), 7.16 (d, J = 7.9 Hz, 1H), 5.88 (br t, J = 7.0 Hz, 1H), 3.93 (s, 3H), 3.72 (s, 2H), 2.73 (d, J = 4.2 Hz, 3H), 2.25 (s, 3H), 1.62 (br d, J = 6.4 Hz, 3H). LCMS (ESI): m/z = 456.2, [M + H]⁺. |
| | (aminomethyl)-2- | | |
| | methylbenzamido)ethyl) | | |
| | quinolin-2-yl)-N,1-dimethyl- | | |
| | 1H-pyrrole-2-carboxamide | | |
| Ex. 258 | 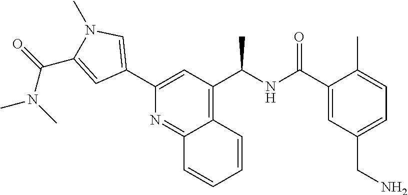 [SMILES: Cc1ccc(CN)cc1C(=O)N[C@H](C)c1cc(-c2cc(C(=O)N(C)C)n(C)c2)nc2ccccc12] (R)-4-(4-(1-(5- (aminomethyl)-2- methylbenzamido)ethyl) quinolin-2-yl)-N,N,1- trimethyl-1H-pyrrole-2- carboxamide | Step 1: methyl (R)- 4-(4-(1-(5-(((tert- butoxycarbonyl) amino)methyl)-2- methylbenzamido) ethyl)quinolin-2-yl)- 1-methyl-1H-pyrrole- 2-carboxylate and NaOH instead of LiOH. Step 2: (R)-4-(4-(1- (5-(((tert- butoxycarbonyl) amino)methyl)-2- methylbenzamido) ethyl)quinolin-2-yl)- 1-methyl-1H-pyrrole- 2-carboxylic acid and Me₂NH. | ¹H NMR (400 MHz, MeOD-d₄) δ = 8.26 (d, J = 8.4 Hz, 1H), 8.05 (d, J = 8.4 Hz, 1H), 7.86 (s, 1H), 7.74 (t, J = 7.7 Hz, 1H), 7.66 (d, J = 1.8 Hz, 1H), 7.62 − 7.55 (m, 1H), 7.36 − 7.32 (m, 2H), 7.24 (d, J = 7.7 Hz, 1H), 7.17 (d, J = 1.8 Hz, 1H), 6.03 (q, J = 7.1 Hz, 1H), 3.85 − 3.81 (m, 5H), 3.25 (br s, 6H), 2.34 (s, 3H), 1.74 (d, J = 7.0 Hz, 3H). LCMS (ESI): m/z = 470.2 [M + H]⁺. |

### Examples 230-231

The following examples were prepared in a similar manner to Example 75 using appropriate reactants.

| Ex. | Structure; | | |
|---|---|---|---|
| # | Compound Name | Reactants used | Characterizing Data |
| Ex. 230 | 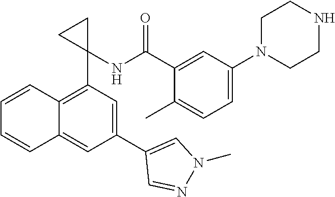 [SMILES: Cc1ccc(N2CCNCC2)cc1C(=O)NC1(c2cc(-c3cnn(C)c3)cc3ccccc23)CC1] | Step 3: 1-(3- bromonaphthalen-1- yl) cyclopropan-1- amine and 5-(4-(tert- butoxycarbonyl) piperazin-1-yl)-2- methylbenzoic acid. Step 4: 1-methyl-4- (4,4,5,5-tetramethyl- 1,3,2-dioxaborolan- 2-yl)-1H-pyrazole. | ¹H NMR (400 MHz, DMSO-d₆) δ = 9.02 (s, 1H), 8.64 − 8.57 (m, 1H), 8.28 (s, 1H), 8.02 − 7.93 (m, 3H), 7.93 − 7.84 (m, 1H), 7.55 − 7.43 (m, 2H), 6.95 (d, J = 8.5 Hz, 1H), 6.80 (dd, J = 2.5, 8.4 Hz, 1H), 6.55 (d, J = 2.4 Hz, 1H), 3.91 (s, 3H), 2.95 − 2.73 (m, 8H), 1.95 (s, 3H), 1.41 − 1.21 (m, 4H). LCMS (ESI): m/z = 466.4, [M + H]⁺. |
| | 2-methyl-N-(1-(3-(1- | | |
| | methyl-1H-pyrazol-4- | | |
| | yl)naphthalen-1- | | |
| | yl) cyclopropyl)-5- | | |
| | (piperazin-1- | | |
| | yl)benzamide | | |
| Ex. 231 | 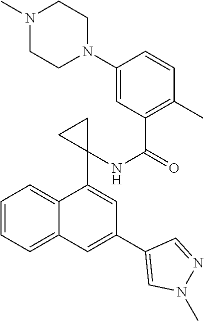 [SMILES: Cc1ccc(N2CCN(C)CC2)cc1C(=O)NC1(c2cc(-c3cnn(C)c3)cc3ccccc23)CC1] | Step 3: 1-(3- bromonaphthalen-1- yl)cyclopropan-1- amine and 2-methyl- 5-(4-methylpiperazin- 1-yl)benzoic acid. Step 4: 1-methyl-4- (4,4,5,5-tetramethyl- 1,3,2-dioxaborolan- 2-yl)-1H-pyrazole. | ¹H NMR (400 MHz, DMSO-d₆) δ = 9.03 (s, 1H), 8.61 (br d, J = 9.4 Hz, 1H), 8.29 (s, 1H), 8.01 − 7.95 (m, 3H), 7.90 − 7.86 (m, 1H), 7.52 − 7.46 (m, 2H), 6.95 (d, J = 8.5 Hz, 1H), 6.82 (dd, J = 2.6, 8.4 Hz, 1H), 6.56 (d, J = 2.5 Hz, 1H), 3.91 (s, 3H), 3.01 − 2.96 (m, 4H), 2.42 − 2.36 (m, 4H), 2.19 (s, 3H), 1.94 (s, 3H), 1.36 (br s, 2H), 1.25 (br s, 2H). LCMS (ESI): m/z = 480.5, [M + H]+ |
| | 2-methyl-N-(1-(3-(1- | | |
| | methyl-1H-pyrazol-4- | | |
| | yl)naphthalen-1- | | |
| | yl)cyclopropyl)-5-(4- | | |
| | methylpiperazin-1- | | |
| | yl)benzamide | | |

### Example 236: (R)-2-methyl-N-(1-(2-(1-methyl-1H-pyrazol-4-yl)quinolin-4-yl)ethyl)-4-(2-oxo-2-(thiazol-4-ylamino)ethyl)benzamide

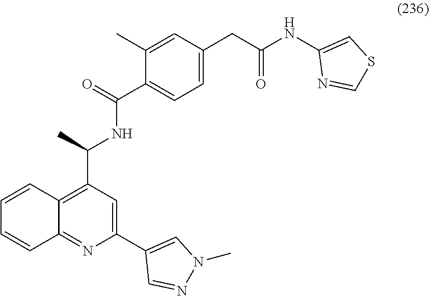 [SMILES: Cc1cc(CC(=O)Nc2cscn2)ccc1C(=O)N[C@H](C)c1cc(-c2cnn(C)c2)nc2ccccc12]

To the solution of (R)-2-(3-methyl-4-((1-(2-(1-methyl-1H-pyrazol-4-yl)quinolin-4-yl)ethyl)carbamoyl)phenyl)acetic acid (100 mg, 0.233 mmol) in pyridine (3.0 mL) were added EDCI (67.1 mg, 0.350 mmol) and thiazol-4-amine (23.4 mg 0.233 mmol) at 20° C. The reaction mixture was stirred at 30° C. for 16 h, at which time the LCMS showed a mass peak of the desired product. The reaction mixture was evaporated in vacuo to remove the solvent, and the residue was dissolved in DMF (2 mL) and purtified by prep-HPLC to afford (R)-2-methyl-N-(1-(2-(1-methyl-1H-pyrazol-4-yl)quinolin-4-yl)ethyl)-4-(2-oxo-2-(thiazol-4-ylamino)ethyl)benzamide (42.06 mg, 35.3%) as a white solid. ¹H NMR (400 MHz, DMSO-d₆) δ=11.26 (s, 1H), 8.97 (d, J=2.4 Hz, 1H), 8.92 (d, J=7.3 Hz, 1H), 8.41 (s, 1H), 8.22 (d, J=8.1 Hz, 1H), 8.09 (s, 1H), 7.97 (d, J=7.9 Hz, 1H), 7.84 (s, 1H), 7.74 (t, J=7.6 Hz, 1H), 7.61-7.54 (m, 2H), 7.33 (d, J=7.6 Hz, 1H), 7.26-7.19 (m, 2H), 5.88 (quin, J=7.1 Hz, 1H), 3.94 (s, 3H), 3.68 (s, 2H), 2.27 (s, 3H), 1.60 (d, J=7.0 Hz, 3H). LCMS (ESI): m/z=511.4, [M+H]⁺. ee %=100% by SFC.

### Prep-HPLC Condition:

Column: Welch Xtimate C18 150*25 mm*5 um;
Mobile phase: water (ammonium hydroxide)-MeCN; B %: 35%-55%,
Flow Rate (mL/min): 60 mL/min.

### Examples 237-239 and 259-260

The following examples were prepared in a similar manner to Example 236 using appropriate reactants.

| Ex. | Structure; | | |
|---|---|---|---|
| # | Compound Name | Reactants used | Characterizing Data |
| Ex. 237 | 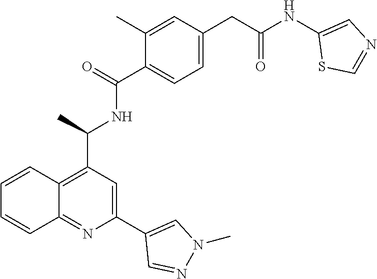 [SMILES: Cc1cc(CC(=O)Nc2cncs2)ccc1C(=O)N[C@H](C)c1cc(-c2cnn(C)c2)nc2ccccc12] | Thiazol-5-amine and (R)-2-(3- methyl-4-((1-(2- (1-methyl-1H- pyrazol-4- yl)quinolin-4- yl)ethyl) carbamoyl) phenyl)acetic acid. | ¹H NMR (400 MHz, DMSO-d₆) δ = 11.62 (s, 1H), 8.93 (d, J = 7.8 Hz, 1H), 8.56 (s, 1H), 8.42 (s, 1H), 8.22 (d, J = 8.1 Hz, 1H), 8.09 (s, 1H), 7.97 (d, J = 7.8 Hz, 1H), 7.85 (s, 1H), 7.73 (t, J = 7.3 Hz, 1H), 7.62 (s, 1H), 7.57 (t, J = 7.6 Hz, 1H), 7.34 (d, J = 7.6 Hz, 1H), 7.20 (s, 1H), 7.21 (d, J = 9.0 Hz, 1H), 5.88 (quin, J = 7.1 Hz, 1H), 3.94 (s, 3H), 3.70 (s, 2H), 2.28 (s, 3H), 1.61 (d, J = 6.9 Hz, 3H). LCMS (ESI): m/z = 511.4, [M + H]⁺. ee % = 100% by SFC. |
| | (R)-2-methyl-N-(1-(2-(1- | | |
| | methyl-1H-pyrazol-4- | | |
| | yl)quinolin-4-yl)ethyl)-4-(2- | | |
| | oxo-2-(thiazol-5- | | |
| | ylamino)ethyl)benzamide | | |
| Ex 238 | 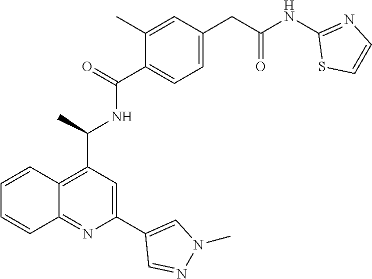 [SMILES: Cc1cc(CC(=O)Nc2nccs2)ccc1C(=O)N[C@H](C)c1cc(-c2cnn(C)c2)nc2ccccc12] | Thiazol-2-amine and (R)-2-(3- methyl-4-((1-(2- (1-methyl-1H- pyrazol-4- yl)quinolin-4- yl)ethyl) carbamoyl) phenyl)acetic acid. | ¹H NMR (400 MHz, DMSO-d₆) δ = 12.36 (s, 1H), 8.93 (d, J = 7.9 Hz, 1H), 8.42 (s, 1H), 8.22 (d, J = 8.3 Hz, 1H), 8.09 (s, 1H), 7.97 (d, J = 8.4 Hz, 1H), 7.84 (s, 1H), 7.73 (t, J = 7.3 Hz, 1H), 7.57 (t, J = 7.6 Hz, 1H), 7.48 (d, J = 3.5 Hz, 1H), 7.34 (d, J = 7.8 Hz, 1H), 7.26 − 7.17 (m, 3H), 5.88 (quin, J = 7.2 Hz, 1H), 3.94 (s, 3H), 3.76 (s, 2H), 2.34 − 2.21 (m, 3H), 1.60 (d, J = 7.0 Hz, 3H). LCMS (ESI): m/z = 511.2, [M + H]⁺. ee % = 100% by SFC. |
| | (R)-2-methyl-N-(1-(2-(1- | | |
| | methyl-1H-pyrazol-4- | | |
| | yl)quinolin-4-yl)ethyl)-4-(2- | | |
| | oxo-2-(thiazol-2- | | |
| | ylamino)ethyl)benzamide | | |
| Ex. 239 | 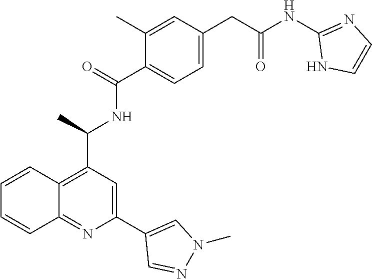 [SMILES: Cc1cc(CC(=O)Nc2ncc[nH]2)ccc1C(=O)N[C@H](C)c1cc(-c2cnn(C)c2)nc2ccccc12] | 1H-imidazol-2- amine and (R)-2- (3-methyl-4-((1- (2-(1-methyl-1H- pyrazol-4- yl)quinolin-4- yl)ethyl) carbamoyl) phenyl)acetic acid. | ¹H NMR (400 MHz, DMSO-d₆) δ = 8.92 (d, J = 7.9 Hz, 1H), 8.41 (s, 1H), 8.24 − 8.17 (m, 1H), 8.09 (s, 1H), 7.97 (d, J = 7.9 Hz, 1H), 7.84 (s, 1H), 7.74 (t, J = 7.3 Hz, 1H), 7.58 (t, J = 7.1 Hz, 1H), 7.33 (d, J = 7.9 Hz, 1H), 7.21 (s, 1H), 7.23 (d, J = 8.6 Hz, 1H), 6.69 (br s, 2H), 5.88 (s, 1H), 3.94 (S, 3H), 3.65 (s, 2H), 2.27 (s, 3H), 1.60 (d, J = 6.9 Hz, 3H). LCMS (ESI): m/z = 494.4, [M + H]⁺. ee % = 100% by SFC. |
| | (R)-4-(2-((1H-imidazol-2- | | |
| | yl)amino)-2-oxoethyl)-2- | | |
| | methyl-N-(1-(2-(1-methyl- | | |
| | 1H-pyrazol-4-yl)quinolin-4- | | |
| | yl)ethyl)benzamide | | |
| Ex. 259 | 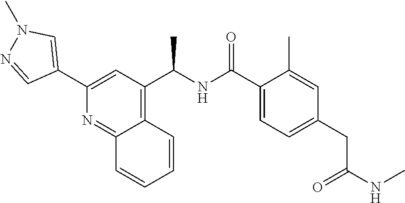 [SMILES: CNC(=O)Cc1ccc(C(=O)N[C@H](C)c2cc(-c3cnn(C)c3)nc3ccccc23)c(C)c1] | MeNH2 and (R)- 2-(3-methyl-4- ((1-(2-(1-methyl- 1H-pyrazol-4- yl)quinolin-4- yl)ethyl) carbamoyl) phenyl)acetic acid. | ¹H NMR (400 MHz, MeOD-d₄) δ = 8.35 (s, 1H), 8.29 (d, J = 8.1 Hz, 1H), 8.18 (s, 1H), 8.07 (d, J = 8.4 Hz, 1H), 7.99 (br s, 1H), 7.87 (s, 1H), 7.78 (t, J = 7.3 Hz, 1H), 7.66 − 7.60 (m, 1H), 7.33 (d, J = 8.4 Hz, 1H), 7.20 − 7.14 (m, 2H), 6.01 (q, J = 6.9 Hz, 1H), 4.00 (s, 3H), 3.47 (s, 2H), 2.75 − 2.65 (m, 3H), 2.32 (s, 3H), 1.72 (d, J = 7.0 Hz, 3H). LCMS (ESI): m/z = 442.4, [M + H]⁺. |
| | (R)-2-methyl-N-(1-(2-(1- | | |
| | methyl-1H-pyrazol-4- | | |
| | yl)quinolin-4-yl)ethyl)-4-(2- | | |
| | (methylamino)-2- | | |
| | oxoethyl)benzamide | | |
| Ex. 260 | 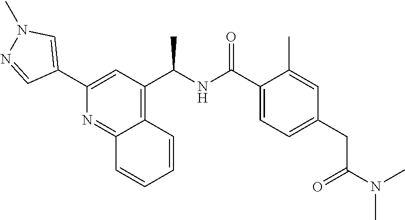 [SMILES: Cc1cc(CC(=O)N(C)C)ccc1C(=O)N[C@H](C)c1cc(-c2cnn(C)c2)nc2ccccc12] | Me2NH and (R)- 2-(3-methyl-4- ((1-(2-(1-methyl- 1H-pyrazol-4- yl)quinolin-4- yl)ethyl) carbamoyl) phenyl)acetic acid. | ¹H NMR (400 MHz, MeOD-d₄) δ = 8.32 (s, 1H), 8.26 (d, J = 8.4 Hz, 1H), 8.16 (s, 1H), 8.05 (d, J = 8.4 Hz, 1H), 7.85 (s, 1H), 7.76 (t, J = 7.7 Hz, 1H), 7.60 (t, J = 7.6 Hz, 1H), 7.33 (d, J = 7.7 Hz, 1H), 7.16 − 7.09 (m, 2H), 6.01 (q, J = 7.0 Hz, 1H), 4.07 − 3.94 (m, 3H), 3.75 (s, 2H), 3.05 (s, 3H), 2.95 (s, 3H), 2.33 (s, 3H), 1.72 (d, J = 7.0 Hz, 3H). LCMS (ESI): m/z = 456.3, [M + H]⁺. |
| | (R)-4-(2-(dimethylamino)- | | |
| | 2-oxoethyl)-2-methyl-N-(1- | | |
| | (2-(1-methyl-1H-pyrazol-4- | | |
| | yl)quinolin-4- | | |
| | yl)ethyl)benzamide | | |

### Example 251: 5-(3,6-diazabicyclo[3.1.1]heptan-3-yl)-2-methyl-N—((R)-1-(2-(1-methyl-1H-pyrazol-4-yl)quinolin-4-yl)ethyl)benzamide

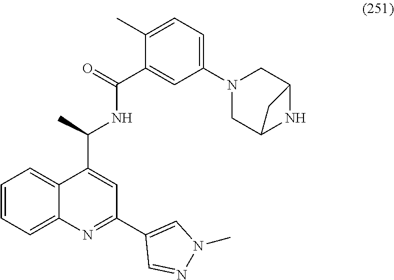 [SMILES: Cc1ccc(N2CC3CC(C2)N3)cc1C(=O)N[C@H](C)c1cc(-c2cnn(C)c2)nc2ccccc12]

### Step 1. Preparation of tert-butyl 3-(4-methyl-3-(((R)-1-(2-(1-methyl-1H-pyrazol-4-yl)quinolin-4-yl)ethyl)carbamoyl)phenyl)-3,6-diazabicyclo[3.1.1]heptane-6-carboxylate

To the solution of 5-(6-(tert-butoxycarbonyl)-3,6-diazabicyclo[3.1.1]heptan-3-yl)-2-methylbenzoic acid (200.0 mg, 0.602 mmol) in pyridine (8.0 mL) was added EDCI (173 mg, 0.903 mmol) and (R)-1-(2-(1-methyl-1H-pyrazol-4-yl)quinolin-4-yl)ethan-1-amine (228 mg, 0.903 mmol) at 20° C. The reaction mixture was stirred at 20° C. for 16 h. The reaction was evaporated to remove the solvent and purified by prep-HPLC to afford a crude tert-butyl 3-(4-methyl-3-(((R)-1-(2-(1-methyl-1H-pyrazol-4-yl)quinolin-4-yl)ethyl)carbamoyl)phenyl)-3,6-diazabicyclo[3.1.1]heptane-6-carboxylate (200 mg) as a white solid after lyophilization.

### Prep-HPLC Condition:

Column: Boston Prime C18 150*30 mm*5 μm;
Mobile phase: water (ammonium hydroxide)-MeCN; B %: 41%-81%,
Flow Rate (mL/min): 60 mL/min.
LCMS of compound 4: LCMS (ESI): m/z=567.3, [M+H]+.

### Step 2. Preparation of 5-(3,6-diazabicyclo[3.1.1]heptan-3-yl)-2-methyl-N—((R)-1-(2-(1-methyl-1H-pyrazol-4-yl)quinolin-4-yl)ethyl)benzamide

To a solution of the crude tert-butyl 3-(4-methyl-3-(((R)-1-(2-(1-methyl-1H-pyrazol-4-yl)quinolin-4-yl)ethyl)carbamoyl)phenyl)-3,6-diazabicyclo[3.1.1]heptane-6-carboxylate (200.0 mg, 0.353 mmol) in DCM (4.0 mL) was added HCl/dioxane (4.0 mL) at 25° C. After addition, the solution was stirred at room temperature (25° C.) for 2 h. The reaction was evaporated in vacuo to give a crude (220 mg) as a yellow solid. 20 mg of the crude was purified by prep-HPLC to afford 5-(3,6-diazabicyclo[3.1.1]heptan-3-yl)-2-methyl-N—((R)-1-(2-(1-methyl-1H-pyrazol-4-yl)quinolin-4-yl)ethyl)benzamide (4.37 mg) as a white solid after lyophilization. ¹H NMR (400 MHz, DMSO-d₆) δ=8.87 (d, J=7.8 Hz, 1H), 8.40 (s, 1H), 8.23 (d, J=7.9 Hz, 1H), 8.07 (s, 1H), 7.99-7.94 (m, 1H), 7.89 (s, 1H), 7.76-7.69 (m, 1H), 7.61-7.54 (m, 1H), 7.07 (d, J=9.0 Hz, 1H), 6.68 (s, 1H), 6.66 (s, 1H), 5.86-5.90 (m, 1H), 3.93 (s, 3H), 3.72 (br d, J=5.7 Hz, 2H), 3.53-3.36 (m, 6H), 2.17 (s, 3H), 1.61 (d, J=7.0 Hz, 3H), 1.48 (d, J=8.2 Hz, 1H). LCMS (ESI): m/z=467.4, [M+H]⁺.

### Prep-HPLC Condition:

Column: Boston Prime C18 150*40 mm*10 μm;
Mobile phase: water (ammonium hydroxide)-MeCN; B %: 26%-66%
Flow Rate (mL/min): 60 mL/min

### Example 252: 2-methyl-N—((R)-1-(2-(1-methyl-1H-pyrazol-4-yl)quinolin-4-yl)ethyl)-5-(6-methyl-3,6-diazabicyclo[3.1.1]heptan-3-yl)benzamide

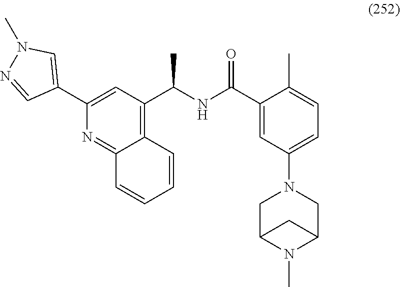 [SMILES: Cc1ccc(N2CC3CC(C2)N3C)cc1C(=O)N[C@H](C)c1cc(-c2cnn(C)c2)nc2ccccc12]

To a solution of 5-(3,6-diazabicyclo[3.1.1]heptan-3-yl)-2-methyl-N—((R)-1-(2-(1-methyl-1H-pyrazol-4-yl)quinolin-4-yl)ethyl)benzamide (150.0 mg, 0.321 mmol) in MeOH (15.0 mL) at 20° C. were added aq. HCHO (1.5 mL), NaBH₃CN (40.4 mg, 0.643 mmol), and HOAc (0.9 mL). The reaction was stirred for 1 h and then evaporated in vacuo to give a crude, which was purified by prep-HPLC to give 2-methyl-N—((R)-1-(2-(1-methyl-1H-pyrazol-4-yl)quinolin-4-yl)ethyl)-5-(6-methyl-3,6-diazabicyclo[3.1.1]heptan-3-yl)benzamide (61.0 mg, 39.5%) as a white solid after lyophilization. ¹H NMR (400 MHz, MeOD-d₄) δ=8.49 (br s, 1H), 8.32 (s, 1H), 8.28 (d, J=8.1 Hz, 1H), 8.15 (s, 1H), 8.05 (dd, J=0.6, 8.4 Hz, 1H), 7.87 (s, 1H), 7.76 (ddd, J=1.2, 7.0, 8.4 Hz, 1H), 7.61 (ddd, J=1.2, 7.0, 8.3 Hz, 1H), 7.17 (d, J=8.4 Hz, 1H), 6.83 (dd, J=2.7, 8.4 Hz, 1H), 6.78 (s, 1H), 6.00-6.06 (m, 1H), 4.27 (br s, 2H), 3.99 (s, 3H), 3.94-3.60 (m, 4H), 3.13 (br s, 1H), 2.92-2.76 (m, 1H), 2.43 (br s, 2H), 2.27 (s, 3H), 1.98-1.89 (m, 1H), 1.74 (d, J=7.0 Hz, 3H). LCMS (ESI): m/z=481.4, [M+H]⁺.

### Prep-HPLC Condition:

Column: Boston Prime C18 150*40 mm*10 μm;
Mobile phase: water (ammonium hydroxide)-MeCN; B %: 0%-34%,
Flow Rate (mL/min): 60 mL/min.

### Example 263: methyl(R)-5-(4-(1-(5-(aminomethyl)-2-methylbenzamido)ethyl)quinolin-2-yl)-1-methyl-1H-pyrrole-3-carboxylate

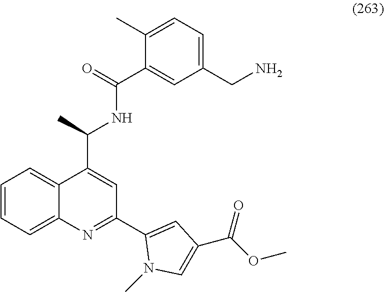 [SMILES: COC(=O)c1cc(-c2cc([C@@H](C)NC(=O)c3cc(CN)ccc3C)c3ccccc3n2)n(C)c1]

### Step 1. Preparation of methyl(R)-5-(4-(1-(5-(((tert-butoxycarbonyl)amino)methyl)-2-methylbenzamido)ethyl)quinolin-2-yl)-1H-pyrrole-3-carboxylate

Using a similar procedure to Step 1 of Example 4 from 5-(((tert-butoxycarbonyl)amino)methyl)-2-methylbenzoic acid (53.9 mg, 0.203 mmol) and methyl(R)-5-(4-(1-aminoethyl)quinolin-2-yl)-1H-pyrrole-3-carboxylate (60.0 mg, 0.20 mmol) affords methyl(R)-5-(4-(1-(5-(((tert-butoxycarbonyl)amino)methyl)-2-methylbenzamido)ethyl)quinolin-2-yl)-1H-pyrrole-3-carboxylate (103 mg, 93%) as a yellow gum. LCMS (ESI): m/z=487.5, [M+H−tBu]⁺.

### Step 2. Preparation of methyl(R)-5-(4-(1-(5-(((tert-butoxycarbonyl)amino)methyl)-2-methylbenzamido)ethyl)quinolin-2-yl)-1-methyl-1H-pyrrole-3-carboxylate

To a solution of methyl(R)-5-(4-(1-(5-(((tert-butoxycarbonyl)amino)methyl)-2-methylbenzamido)ethyl)quinolin-2-yl)-1H-pyrrole-3-carboxylate (110 mg, 0.203 mmol) in DMF (2.03 mL) at 25° C. was added Cs₂CO₃ (66.0 mg, 0.203 mmol). The mixture was stirred for 1 h before CH₃I (31.7 mg, 0.223 mmol) was added to it. The reaction mixture was the stirred at 25° C. for an 16 h. The reaction mixture was evaporated in vacuo to give a crude, which was dissolved in ethyl acetate (5 mL) and H₂O (10 mL) and then extracted with ethyl acetate (5 mL×3). The combined organic layer was dried with Na₂SO₄ and filtered to give methyl(R)-5-(4-(1-(5-(((tert-butoxycarbonyl)amino)methyl)-2-methylbenzamido)ethyl)quinolin-2-yl)-1-methyl-1H-pyrrole-3-carboxylate (110 mg, 97.5%) as a yellow gum. LCMS (ESI): m/z=501.2, [M+H−tBu]⁺.

### Step 3. Preparation of methyl(R)-5-(4-(1-(5-(aminomethyl)-2-methylbenzamido)ethyl)quinolin-2-yl)-1-methyl-1H-pyrrole-3-carboxylate

Using a procedure similar to Step 2 of Example 4 or Step 2 of Example 51 from methyl(R)-5-(4-(1-(5-(((tert-butoxycarbonyl)amino)methyl)-2-methylbenzamido)ethyl)quinolin-2-yl)-1-methyl-1H-pyrrole-3-carboxylate (100 mg, 0.180 mmol) affords methyl(R)-5-(4-(1-(5-(aminomethyl)-2-methylbenzamido)ethyl)quinolin-2-yl)-1-methyl-1H-pyrrole-3-carboxylate (31.28 mg, 38.1%) as a white solid. ¹H NMR (400 MHz, DMSO-d₆, 300 K) Shift (ppm)=9.01 (br d, J=7.9 Hz, 1H), 8.27 (d, J=8.3 Hz, 1H), 8.06-8.01 (m, 2H), 7.78 (t, J=7.5 Hz, 1H), 7.72 (s, 1H), 7.67-7.60 (m, 1H), 7.41-7.32 (m, 2H), 7.27-7.20 (m, 3H), 5.95 (br t, J=7.1 Hz, 1H), 4.21 (s, 3H), 4.15 (br s, 1H), 3.80 (s, 2H), 3.75 (s, 3H), 2.55 (s, 1H), 2.27-2.22 (m, 3H), 1.59 (br d, J=7.0 Hz, 3H). LCMS (ESI): m/z=457.4, [M+H]⁺.

### Example 267: (R)-2-methyl-N-(1-(2-(1-methyl-1H-pyrazol-4-yl)quinolin-4-yl)ethyl)-4-((N-(thiazol-4-ylmethyl)acetamido)methyl)benzamide

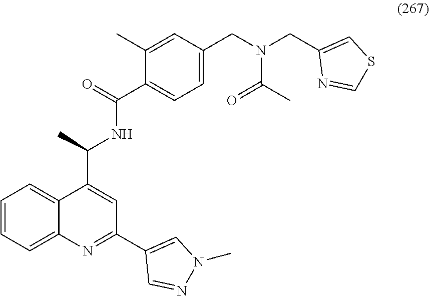 [SMILES: CC(=O)N(Cc1ccc(C(=O)N[C@H](C)c2cc(-c3cnn(C)c3)nc3ccccc23)c(C)c1)Cc1cscn1]

To solution of (R)-2-methyl-N-(1-(2-(1-methyl-1H-pyrazol-4-yl)quinolin-4-yl)ethyl)-4-(((thiazol-4-ylmethyl)amino)methyl)benzamide (55.0 mg, 0.11 mmol) in DCM (5 mL) were added Ac₂O (33.9 mg, 0.332 mmol) and TEA (33.6 mg, 0.332 mmol). The reaction mixture was stirred at 20° C. for 2 h then purified by prep-HPLC to afford (R)-2-methyl-N-(1-(2-(1-methyl-1H-pyrazol-4-yl)quinolin-4-yl)ethyl)-4-((N-(thiazol-4-ylmethyl)acetamido)methyl)benzamide (37 mg, 62%) as a white solid after lyophilization. ¹H NMR of PLASMA-718: ¹H NMR (400 MHz, MeOD-d₄) δ=9.02-8.94 (m, 1H), 8.35 (s, 1H), 8.29 (d, J=8.4 Hz, 1H), 8.18 (s, 1H), 8.08 (d, J=8.4 Hz, 1H), 7.86 (s, 1H), 7.78 (t, J=7.7 Hz, 1H), 7.63 (t, J=7.7 Hz, 1H), 7.44 (s, 1H), 7.42-7.33 (m, 1H), 7.15-7.09 (m, 2H), 6.04 (br d, J=7.0 Hz, 1H), 4.77-4.69 (m, 2H), 4.67-4.63 (m, 2H), 4.02 (s, 3H), 2.37-2.32 (m, 5H), 2.18 (s, 1H), 1.75 (d, J=6.8 Hz, 3H). LCMS (ESI): m/z=539.3, [M+H]⁺.

### Prep-HPLC Condition:

Column: Boston Prime C₁₈-1 150*30 mm*5 μm;
Mobile phase: water (ammonium hydroxide)-MeCN; B %: 17%-57%,
Flow Rate (mL/min): 30 mL/min.

### Example 268: (R)-2-methyl-N-(1-(2-(1-methyl-1H-pyrazol-4-yl)quinolin-4-yl)ethyl)-4-((N-(thiazol-4-ylmethyl)methylsulfonamido)methyl)benzamide

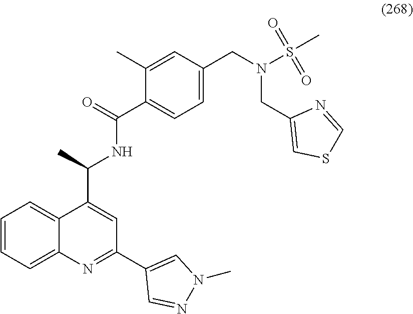 [SMILES: Cc1cc(CN(Cc2cscn2)S(C)(=O)=O)ccc1C(=O)N[C@H](C)c1cc(-c2cnn(C)c2)nc2ccccc12]

To solution of (R)-2-methyl-N-(1-(2-(1-methyl-1H-pyrazol-4-yl)quinolin-4-yl)ethyl)-4-(((thiazol-4-ylmethyl)amino)methyl)benzamide (55.0 mg, 0.11 mmol) in DCM (5 mL) were added Ms₂O (57.9 mg, 0.332 mmol) and TEA (33.6 mg, 0.332 mmol). The reaction mixture was stirred at 20° C. for 2 h then purified by prep-HPLC to afford (R)-2-methyl-N-(1-(2-(1-methyl-1H-pyrazol-4-yl)quinolin-4-yl)ethyl)-4-((N-(thiazol-4-ylmethyl)methylsulfonamido)methyl)benzamide (34 mg, 53%) as a white solid after lyophilization. ¹H NMR (400 MHz, MeOD-d₄) δ=8.99 (d, J=1.9 Hz, 1H), 8.35 (s, 1H), 8.29 (d, J=8.1 Hz, 1H), 8.19 (s, 1H), 8.08 (d, J=7.9 Hz, 1H), 7.87 (s, 1H), 7.79 (t, J=7.7 Hz, 1H), 7.63 (ddd, J=1.2, 7.0, 8.3 Hz, 1H), 7.44 (d, J=2.0 Hz, 1H), 7.38 (d, J=7.9 Hz, 1H), 7.27 (d, J=7.9 Hz, 1H), 7.23 (s, 1H), 6.04 (q, J=6.9 Hz, 1H), 4.50 (s, 2H), 4.42 (s, 2H), 4.02 (s, 3H), 2.97 (s, 3H), 2.36 (s, 3H), 1.75 (d, J=7.0 Hz, 3H). LCMS (ESI): m/z=575.2, [M+H]⁺.

### Prep-HPLC Condition:

Column: Boston Prime C₁₈-1 150*30 mm*5 μm;
Mobile phase: water (ammonium hydroxide)-MeCN; B %: 22%-62%,
Flow Rate (mL/min): 30 mL/min.

### Examples 264-265

The following examples were prepared in a similar manner to Example 54 using an appropriate amine and an appropriate carboxylic acid.

| Ex. | Structure; | Reactants | |
|---|---|---|---|
| # | Compound Name | used | Characterizing Data |
| Ex. 264 | 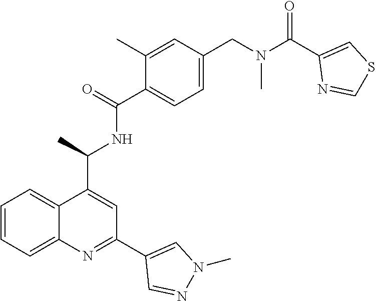 [SMILES: Cc1cc(CN(C)C(=O)c2cscn2)ccc1C(=O)N[C@H](C)c1cc(-c2cnn(C)c2)nc2ccccc12] | (R)-2-methyl- N-(1-(2-(1- methyl-1H- pyrazol-4- yl)quinolin-4- yl)ethyl)-4- ((methylamino) methyl) benzamide and thiazole-4- carboxylic acid | ¹H NMR (400 MHz, MeOD-d₄) δ = 9.14 − 8.97 (m, 1H), 8.37 (s, 1H), 8.31 (br d, J = 8.6 Hz, 1H), 8.20 (s, 1H), 8.14 (d, J = 2.0 Hz, 1H), 8.09 (d, J = 8.4 Hz, 1H), 7.89 (br d, J = 3.3 Hz, 1H), 7.81 (br t, J = 7.6 Hz, 1H), 7.70 − 7.61 (m, 1H), 7.45 − 7.36 (m, 1H), 7.33 − 7.25 (m, 1H), 7.23 − 7.16 (m, 1H), 6.04 (br d, J = 6.2 Hz, 1H), 4.94 − 4.92 (m, 1H), 4.82 − 4.75 (m, 1H), 4.02 (s, 3H), 3.20 − 2.91 (m, 3H), 2.37 (br d, J = 10.9 Hz, 3H), 1.75 (br d, J = 6.2 Hz, 3H). LCMS (ESI): m/z = 525.3 , [M + H]⁺. |
| | (R)-N-methyl-N-(3-methyl- | | |
| | 4-((1-(2-(1-methyl-1H- | | |
| | pyrazol-4-yl)quinolin-4- | | |
| | yl)ethyl)carbamoyl)benzyl) | | |
| | thiazole-4-carboxamide | | |
| Ex. 265 | 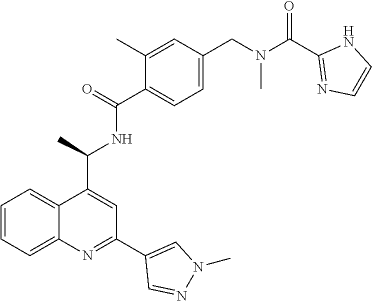 [SMILES: Cc1cc(CN(C)C(=O)c2ncc[nH]2)ccc1C(=O)N[C@H](C)c1cc(-c2cnn(C)c2)nc2ccccc12] | (R)-2-methyl- N-(1-(2-(1- methyl-1H- pyrazol-4- yl)quinolin-4- yl)ethyl)-4- ((methylamino) methyl) benzamide and thiazole-4- carboxylic acid | ¹H NMR (400 MHz, MeOD-d₄) δ = 8.34 (s, 1H), 8.29 (br d, J = 8.2 Hz, 1H), 8.18 (s, 1H), 8.08 (d, J = 8.1 Hz, 1H), 7.86 (s, 1H), 7.78 (t, J = 7.7 Hz, 1H), 7.67 − 7.59 (m, 1H), 7.39 (br t, J = 8.1 Hz, 1H), 7.28 − 7.25 (m, 1H), 7.24 − 7.22 (m, 1H), 7.21 − 7.03 (m, 2H), 6.04 (br d, J = 6.5 Hz, 1H), 5.48 (s, 1H), 4.77 (s, 1H), 4.02 (s, 3H), 3.52 − 3.39 (m, 2H), 2.99 (s, 1H), 2.35 (br d, J = 9.3 Hz, 3H), 1.74 (br d, J = 6.0 Hz, 3H). LCMS (ESI): m/z = 508.4, [M + H]⁺. |
| | (R)-N-methyl-N-(3-methyl- | | |
| | 4-((1-(2-(1-methyl-1H- | | |
| | pyrazol-4-yl)quinolin-4- | | |
| | yl)ethyl)carbamoyl)benzyl)- | | |
| | 1H-imidazole-2- | | |
| | carboxamide | | |

### Example 273: 5-(4-(2-hydroxyethyl)piperazin-1-yl)-2-methyl-N-(1-(2-(1-methyl-1H-pyrazol-4-yl)quinolin-4-yl)cyclopropyl)benzamide

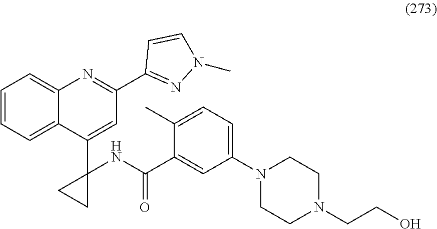 [SMILES: Cc1ccc(N2CCN(CCO)CC2)cc1C(=O)NC1(c2cc(-c3ccn(C)n3)nc3ccccc23)CC1]

To a solution of 2-methyl-N-(1-(2-(1-methyl-1H-pyrazol-4-yl)quinolin-4-yl)cyclopropyl)-5-(piperazin-1-yl)benzamide (60.0 mg, 0.13 mmol) and 2-bromoethan-1-ol (32.1 mg, 0.257 mmol) in DMF (4 mL) was added TEA (13.0 mg, 0.129 mmol) at 20° C. The reaction mixture was stirred at 60° C. for 2 h. After that, the reaction mixture was filtered, and the filtrate was purified by prep-HPLC to afford 5-(4-(2-hydroxyethyl)piperazin-1-yl)-2-methyl-N-(1-(2-(1-methyl-1H-pyrazol-4-yl)quinolin-4-yl)cyclopropyl)benzamide (22.62 mg, 34%) as a white solid after lyophilization. ¹H NMR (400 MHz, METHANOL-d₄) δ=8.56 (d, J=8.2 Hz, 1H), 8.41 (s, 1H), 8.27 (s, 1H), 8.12-8.05 (m, 2H), 7.76 (t, J=7.2 Hz, 1H), 7.61 (t, J=7.2 Hz, 1H), 7.03 (d, J=8.4 Hz, 1H), 6.90 (dd, J=2.6, 8.4 Hz, 1H), 6.68 (d, J=2.6 Hz, 1H), 4.03 (s, 3H), 3.72 (t, J=6.0 Hz, 2H), 3.15-3.05 (m, 4H), 2.69-2.56 (m, 6H), 2.00 (s, 3H), 1.53-1.41 (m, 4H). LCMS (ESI): m/z=511.4, [M+H]⁺.

### Prep-HPLC Condition:

Column: C18 (150*30 mm);
Mobile phase: Water (NH₃·H₂O—NH₄HCO₃)-MeCN; B %: 9%-49%,
Flow rate (mL/min): 30 mL/min.

### Example 274: (R)-2-methyl-N-(1-(2-(1-methyl-1H-pyrazol-4-yl)quinolin-4-yl)ethyl)-4-((thiazol-4-ylmethoxy)methyl)benzamide

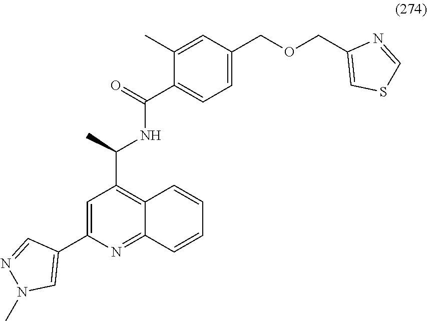 [SMILES: Cc1cc(COCc2cscn2)ccc1C(=O)N[C@H](C)c1cc(-c2cnn(C)c2)nc2ccccc12]

### Step 1: Preparation of methyl 4-formyl-2-methylbenzoate

To a solution of 4-bromo-3-methylbenzaldehyde (4000 mg, 20 mmol) in MeOH (100 mL) were added TEA (6100 mg, 60.3 mmol) and Pd(dppf)Cl₂ (1470 mg, 2.01 mmol) at 15° C. The mixture was bubbled with CO gas for 1 min then stirred at 65° C. for 16 h under 50 psi (3,44738 bar) of CO. After that, the reaction mixture was filtered, and the filtrate was evaporated in vacuo to give a brown oil crude. The crude was then suspended in H₂O (80 mL) at 15° C. and extracted with ethyl acetate (100 mL×3). The organic layer was evaporated in vacuo, then the residue was purified by Biotage (80 g silica gel column, EA/PE from 0 to 60%) to give methyl 4-formyl-2-methylbenzoate (3400 mg, 90%) as a white solid. ¹H NMR (400 MHz, CDCl₃-d) 6 ppm 10.06 (s, 1H), 8.04 (d, J=8.31 Hz, 1H), 7.79-7.72 (m, 2H), 3.95 (s, 3H), 2.68 (s, 3H). LCMS (ESI): m/z=200.4 [M+Na]⁺.

### Step 2: Preparation of methyl 4-(hydroxymethyl)-2-methylbenzoate

To a solution of methyl 4-formyl-2-methylbenzoate (2000 mg, 11.22 mmol) in CH₃OH (50 mL) was added NaBH₄ (425 mg, 11.2 mmol) at 15° C., then the reaction was stirred at 15° C. for 2 h. After that, the reaction mixture was diluted by H₂O (100 mL) at 0° C. and extracted with ethyl acetate (200 mL×3). The organic layer was evaporated in vacuo to give a crude, which was purified by Biotage (80 g silica gel column, EA/PE from 0 to 80%) to give methyl 4-(hydroxymethyl)-2-methylbenzoate (2000 mg, 98.9%) as a white solid. ¹H NMR (400 MHz, CDCl₃-d) δ ppm 8.02-7.87 (m, 1H), 7.33-7.22 (m, 2H), 4.74 (s, 2H), 3.91 (s, 3H), 2.63 (s, 3H).

### Step 3: Preparation of methyl 4-(chloromethyl)-2-methylbenzoate

To a solution of methyl 4-(hydroxymethyl)-2-methylbenzoate (1000 mg, 5.549 mmol) in DCM (40.0 mL) and anhydrous DMF (40.6 mg, 0.555 mmol) was added SOCl₂ (990 mg, 8.32 mmol) at 15° C. in a period of 10 min. The reaction mixture was stirred at 15° C. for 16 h. After that, the reaction mixture was diluted by H₂O (30 mL) and extracted by DCM (50 mL×3). The organic layer was dried over Na₂SO₄, filtered and evaporated in vacuo to give a crude product, which was purified by chromatography (40 g silica gel column, EA/PE from 0 to 100%) to give methyl 4-(chloromethyl)-2-methylbenzoate (1000 mg, 90.7%) as a white solid. ¹H NMR (400 MHz, CDCl₃-d) δ ppm 7.98-7.90 (m, 1H), 7.31-7.26 (m, 2H), 4.58 (s, 2H), 3.91 (s, 3H), 2.63 (s, 3H).

### Step 4: Preparation of methyl 2-methyl-4-((thiazol-4-ylmethoxy)methyl)benzoate

To a solution of thiazol-4-ylmethanol (191 mg, 1.66 mmol) in THF (20 mL) was added NaH (145 mg, 6.04 mmol). The reaction mixture was stirred at 15° C. for 30 min before methyl 4-(chloromethyl)-2-methylbenzoate (300 mg, 1.51 mmol) was added to it. After being stirred at 15° C. for 16 h, the reaction mixture was quenched with H₂O (50 mL) then extracted by ethyl acetate (80 mL×3). The organic layer was separated, washed with brine (30 mL×5), then evaporated in vacuo to give a crude, which was purified by a Biotage (40 g silica gel column, EA/PE from 0 to 15%) to afford methyl 2-methyl-4-((thiazol-4-ylmethoxy)methyl)benzoate (80 mg, 19%) as a yellow oil. LCMS (ESI): m/z=277.8 [M+H]⁺.

### Step 5: Preparation of 2-methyl-4-((thiazol-4-ylmethoxy)methyl)benzoic acid

To a solution of methyl 2-methyl-4-((thiazol-4-ylmethoxy)methyl)benzoate (80 mg, 0.29 mmol) in MeOH (6 mL) and H₂O (2 mL) was added KOH (162 mg 2.88 mmol) at 15° C. The reaction mixture was then stirred at 15° C. for 16 h. After that, the mixture was evaporated in vacuo then redissolved in H₂O (20 mL). The mixture was acidified with HCl (1N) to pH=5-6 and extracted with DCM (50 mL×3). The organic layer was evaporated in vacuo to afford 2-methyl-4-((thiazol-4-ylmethoxy)methyl)benzoic acid (75 mg, 99%) as a white solid. LCMS (ESI): m/z=263.9 [M+H]⁺.

### Step 6: Preparation of (R)-2-methyl-N-(1-(2-(1-methyl-1H-pyrazol-4-yl)quinolin-4-yl)ethyl)-4-((thiazol-4-ylmethoxy)methyl)benzamide

To a solution of 2-methyl-4-((thiazol-4-ylmethoxy)methyl)benzoic acid (85.0 mg, 0.32 mmol) in anhydrous DMF (10 mL) were added HATU (160 mg, 0.42 mmol), DIEA (209 mg, 1.61 mmol), and (R)-1-(2-(1-methyl-1H-pyrazol-4-yl)quinolin-4-yl)ethan-1-amine (89.6 mg, 0.355 mmol) at 15° C. The reaction mixture was stirred at 15° C. for 16 h before being diluted by H₂O (20 mL) and extracted with ethyl acetate (30 mL×3). The combined organic layer was washed with H₂O (30 mL) three times then evaporated in vacuo. The residue was purified by prep-HPLC to afford (R)-2-methyl-N-(1-(2-(1-methyl-1H-pyrazol-4-yl)quinolin-4-yl)ethyl)-4-((thiazol-4-ylmethoxy)methyl)benzamide (29.1 mg, 17%) as a white solid after lyophilization. ¹H NMR (400 MHz, METHANOL-d4) δ ppm 1.77 (d, J=7.13 Hz, 3H), 2.37 (s, 3H), 4.05 (s, 3H), 4.63 (s, 2H), 4.72 (s, 2H), 5.99-6.13 (m, 1H), 7.27-7.34 (m, 2H), 7.42 (d, J=8.13 Hz, 1H), 7.56-7.61 (m, 1H), 7.69-7.79 (m, 1H), 7.86-7.95 (m, 1H), 7.99 (s, 1H), 8.15 (d, J=8.75 Hz, 1H), 8.28 (s, 1H), 8.41 (br d, J=9.01 Hz, 1H), 8.47 (s, 1H), 9.02 (d, J=2.00 Hz, 1H), 9.09 (br d, J=7.63 Hz, 1H). LCMS (ESI): m/z=498.3 [M+H]⁺.

### Prep-HPLC Condition:

Column: YMC Triart 30*150 mm*7 μm;
Mobile phase: water (FA)-MeCN;
Flow rate (mL/min): 25 mL/min.

### Example 275: 2-methyl-N-(1-(2-(3-methyl-1H-pyrazol-5-yl)quinolin-4-yl)cyclopropyl)-5-(4-methylpiperazin-1-yl)benzamide

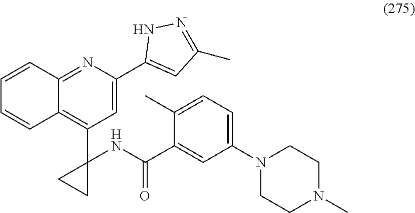 [SMILES: Cc1cc(-c2cc(C3(NC(=O)c4cc(N5CCN(C)CC5)ccc4C)CC3)c3ccccc3n2)[nH]n1]

### Step 1: Preparation of 1-(2-(3-methyl-1H-pyrazol-5-yl)quinolin-4-yl)cyclopropan-1-amine

To a solution of 1-(2-chloroquinolin-4-yl)cyclopropan-1-amine as prepared in step 4 of P14 (100 mg, 0.457 mmol) in 1,4-dioxane (3 mL) and H₂O (1.0 mL) were added (3-methyl-1H-pyrazol-5-yl)boronic acid (57.6 mg, 0.457 mmol) and K₃PO₄ (291 mg, 1.37 mmol). The mixture was then bubbled with N₂ for 3 min before Pd(dppf)Cl₂ (33.5 mg, 0.0457 mmol) was added to it. The mixture was bubbled with N₂ for 3 min again and then stirred at 100° C. for 15 h. After that, the reaction mixture was filtered, and the filtrate was evaporated in vacuo. The residue was purified using a Biotage (4 g silica gel column, MeOH/EtOAc from 0 to 20%) to afford 1-(2-(3-methyl-1H-pyrazol-5-yl)quinolin-4-yl)cyclopropan-1-amine (70 mg, 58%) as a brown solid. ¹H NMR (400 MHz, DMSO-d₆) δ=8.44 (br d, J=8.0 Hz, 1H), 8.01 (br d, J=8.1 Hz, 1H), 7.73 (br t, J=7.3 Hz, 1H), 7.52-7.47 (m, 1H), 7.27-7.08 (m, 2H), 6.75 (br s, 1H), 4.45 (br s, 1H), 4.26 (br s, 1H), 2.31 (s, 3H), 1.23 (br s, 1H), 1.09 (br s, 1H), 0.97-0.83 (m, 2H). LCMS (ESI): m/z=265.1, [M+H]⁺.

### Step 2: Preparation of 2-methyl-N-(1-(2-(3-methyl-1H-pyrazol-5-yl)quinolin-4-yl)cyclopropyl)-5-(4-methylpiperazin-1-yl)benzamide

To a solution of 2-methyl-5-(4-methylpiperazin-1-yl)benzoic acid (71.7 mg, 0.265 mmol) in DMF (3.0 mL) were added HATU (151 mg, 0.397 mmol) and DIEA (103 mg, 0.795 mmol) at 20° C. The mixture was then stirred at 20° C. for 10 min before 1-(2-(3-methyl-1H-pyrazol-5-yl)quinolin-4-yl)cyclopropan-1-amine (70.0 mg, 0.26 mmol) was added to it. After 12 h of stirring at 20° C., the reaction mixture was poured into H₂O (20 mL) at 20° C. and extracted with ethyl acetate (30 mL×3). The combined organic layer was washed with brine (10 mL), dried over Na₂SO₄, filtered, and evaporated in vacuo to give a crude, which was purified by prep-HPLC to afford 2-methyl-N-(1-(2-(3-methyl-1H-pyrazol-5-yl)quinolin-4-yl)cyclopropyl)-5-(4-methylpiperazin-1-yl)benzamide (2.86 mg, 2.2%) as a white solid after lyophilization. ¹H NMR (400 MHz, DMSO-d₆) δ=12.86 (br s, 1H), 9.16 (s, 1H), 8.63 (d, J=8.0 Hz, 1H), 8.30 (br s, 1H), 8.01 (br d, J=8.1 Hz, 1H), 7.82-7.67 (m, 1H), 7.66-7.47 (m, 1H), 6.95 (d, J=8.4 Hz, 1H), 6.83 (dd, J=2.5, 8.5 Hz, 1H), 6.74 (br s, 1H), 6.60 (d, J=2.3 Hz, 1H), 3.03-2.97 (m, 4H), 2.43-2.38 (m, 4H), 2.33 (br s, 3H), 2.20 (s, 3H), 1.91 (s, 3H), 1.39 (br s, 2H), 1.30-1.20 (m, 2H). LCMS (ESI): m/z=481.4, [M+H]⁺.

### Prep-HPLC Condition:

Column: Boston Prime C18 150*30 mm*5 μm;
Mobile phase: water (ammonium hydroxide v/v)-MeCN; B %: 24%-51%,
Flow rate (mL/min): 30 mL/min.

### Example 276: N,N-dimethyl-5-(4-(1-(2-methyl-5-(piperazin-1-yl)benzamido)cyclopropyl) naphthalen-2-yl)-1H-pyrrole-2-carboxamide

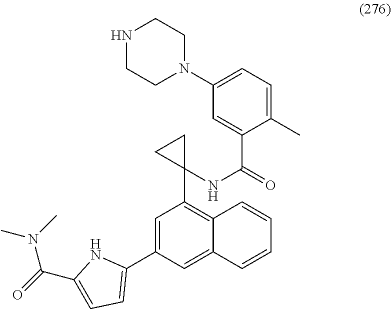

### Step 1: Preparation of tert-butyl 4-(3-((1-(3-bromonaphthalen-1-yl)cyclopropyl)carbamoyl)-4-methylphenyl)piperazine-1-carboxylate

Using a procedure analogous to Step 4 of Example 75 with 5-(4-(tert-butoxycarbonyl)piperazin-1-yl)-2-methylbenzoic acid and 1-(3-bromonaphthalen-1-yl)cyclopropan-1-amine affords tert-butyl 4-(3-((1-(3-bromonaphthalen-1-yl)cyclopropyl)carbamoyl)-4-methylphenyl)piperazine-1-carboxylate. LCMS (ESI): m/z=566.0, [M+H]⁺.

### Step 2: Preparation of tert-butyl 4-(3-((1-(3-(5-(methoxycarbonyl)-1H-pyrrol-2-yl)naphthalen-1-yl)cyclopropyl)carbamoyl)-4-methylphenyl)piperazine-1-carboxylate

Using a procedure analogous to Step 5 of Example 75 with methyl 5-(4,4,5,5-tetramethyl-1,3,2-dioxaborolan-2-yl)-1H-pyrrole-2-carboxylate and tert-butyl 4-(3-((1-(3-bromonaphthalen-1-yl)cyclopropyl)carbamoyl)-4-methylphenyl)piperazine-1-carboxylate affords tert-butyl 4-(3-((1-(3-(5-(methoxycarbonyl)-1H-pyrrol-2-yl)naphthalen-1-yl)cyclopropyl)carbamoyl)-4-methylphenyl)piperazine-1-carboxylate. LCMS (ESI): m/z=609.3, [M+H]⁺.

### Step 3: Preparation of 5-(4-(1-(5-(4-(tert-butoxycarbonyl)piperazin-1-yl)-2-methylbenzamido)cyclopropyl)naphthalen-2-yl)-1H-pyrrole-2-carboxylic acid

Using a procedure analogous to Step 1 of Example 20 with tert-butyl 4-(3-((1-(3-(5-(methoxycarbonyl)-1H-pyrrol-2-yl)naphthalen-1-yl)cyclopropyl)carbamoyl)-4-methylphenyl)piperazine-1-carboxylate affords 5-(4-(1-(5-(4-(tert-butoxycarbonyl)piperazin-1-yl)-2-methylbenzamido)cyclopropyl)naphthalen-2-yl)-1H-pyrrole-2-carboxylic acid (91.0% yield). ¹H NMR (400 MHz, MeOD-d4) δ=8.53 (d, J=7.5 Hz, 1H), 8.19 (d, J=1.5 Hz, 1H), 8.11 (s, 1H), 7.93 (d, J=7.3 Hz, 1H), 7.57-7.48 (m, 2H), 7.03-6.92 (m, 2H), 6.87 (dd, J=2.6, 8.4 Hz, 1H), 6.72 (d, J=4.0 Hz, 1H), 6.66 (d, J=2.6 Hz, 1H), 3.48 (br s, 4H), 3.01-2.92 (m, 4H), 2.00-1.97 (m, 3H), 1.45 (s, 9H), 1.37 (br s, 2H), 1.27 (s, 2H). LCMS (ESI): m/z=595.2, [M+H]⁺.

### Step 4: Preparation of tert-butyl 4-(3-((1-(3-(5-(dimethylcarbamoyl)-1H-pyrrol-2-yl)naphthalen-1-yl)cyclopropyl)carbamoyl)-4-methylphenyl)piperazine-1-carboxylate

Using a procedure analogous to Step 2 of Example 71 with 5-(4-(1-(5-(4-(tert-butoxycarbonyl)piperazin-1-yl)-2-methylbenzamido)cyclopropyl)naphthalen-2-yl)-1H-pyrrole-2-carboxylic acid and dimethylamine hydrochloride affords tert-butyl 4-(3-((1-(3-(5-(dimethylcarbamoyl)-1H-pyrrol-2-yl)naphthalen-1-yl)cyclopropyl)carbamoyl)-4-methylphenyl)piperazine-1-carboxylate (90% yield). LCMS (ESI): m/z=622.3, [M+H]⁺.

### Step 5: Preparation of N,N-dimethyl-5-(4-(1-(2-methyl-5-(piperazin-1-yl)benzamido)cyclopropyl)naphthalen-2-yl)-1H-pyrrole-2-carboxamide

Using a procedure analogous to Step 3 of Example 71 with tert-butyl 4-(3-((1-(3-(5-(dimethylcarbamoyl)-1H-pyrrol-2-yl)naphthalen-1-yl)cyclopropyl)carbamoyl)-4-methylphenyl)piperazine-1-carboxylate affords N,N-dimethyl-5-(4-(1-(2-methyl-5-(piperazin-1-yl)benzamido)cyclopropyl)naphthalen-2-yl)-1H-pyrrole-2-carboxamide (12% yield). ¹H NMR (400 MHz, DMSO-d₆) δ=9.05 (s, 1H), 8.64-8.54 (m, 1H), 8.31 (s, 1H), 8.25 (s, 1H), 8.18 (s, 1H), 7.97-7.86 (m, 1H), 7.56-7.45 (m, 2H), 6.96 (d, J=8.6 Hz, 1H), 6.82 (dd, J=2.6, 8.5 Hz, 1H), 6.73 (d, J=3.8 Hz, 1H), 6.56 (d, J=2.6 Hz, 1H), 3.23-2.93 (m, 10H), 2.87 (br s, 4H), 1.94-1.88 (m, 3H), 1.33 (br d, J=9.7 Hz, 4H). LCMS (ESI): m/z=522.3, [M+H]⁺.

### Prep-HPLC Condition:

Column: Boston Prime C18 150*30 mm*5 μm;
Mobile phase: water (FA)-MeCN; B %: 15%-45%,
Flow rate (mL/min): 30 mL/min.

### Example 277: N,N-dimethyl-5-(4-(1-(2-methyl-5-(4-methylpiperazin-1-yl)benzamido)cyclopropyl)naphthalen-2-yl)-1H-pyrrole-3-carboxamide

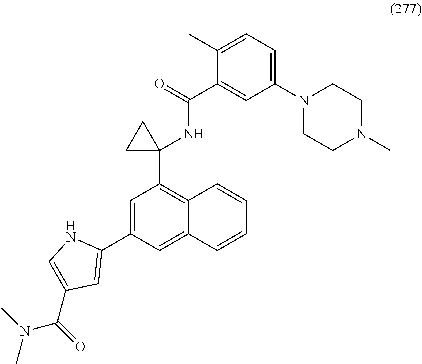

### Step 1: Preparation of N-(1-(3-bromonaphthalen-1-yl)cyclopropyl)-2-methyl-5-(4-methylpiperazin-1-yl)benzamide

Using a procedure analogous to Step 4 of Example 75 with 1-(3-bromonaphthalen-1-yl)cyclopropan-1-amine and 2-methyl-5-(4-methylpiperazin-1-yl)benzoic acid affords N-(1-(3-bromonaphthalen-1-yl)cyclopropyl)-2-methyl-5-(4-methylpiperazin-1-yl)benzamide (45% yield). LCMS (ESI): m/z=478.1, [M+H]⁺.

### Step 2: Preparation of methyl 5-(4-(1-(2-methyl-5-(4-methylpiperazin-1-yl)benzamido)cyclopropyl)naphthalen-2-yl)-1H-pyrrole-3-carboxylate

Using a procedure analogous to Step 5 of Example 75 with methyl 5-(4,4,5,5-tetramethyl-1,3,2-dioxaborolan-2-yl)-1H-pyrrole-3-carboxylate and N-(1-(3-bromonaphthalen-1-yl)cyclopropyl)-2-methyl-5-(4-methylpiperazin-1-yl)benzamide affords methyl 5-(4-(1-(2-methyl-5-(4-methylpiperazin-1-yl)benzamido)cyclopropyl)naphthalen-2-yl)-1H-pyrrole-3-carboxylate (86% yield). LCMS (ESI): m/z=523.2, [M+H]⁺.

### Step 3: Preparation of 5-(4-(1-(2-methyl-5-(4-methylpiperazin-1-yl)benzamido)cyclopropyl)naphthalen-2-yl)-1H-pyrrole-3-carboxylic acid

To a solution of methyl 5-(4-(1-(2-methyl-5-(4-methylpiperazin-1-yl)benzamido)cyclopropyl) naphthalen-2-yl)-1H-pyrrole-3-carboxylate (80.0 mg, 0.15 mmol) in MeOH (4.0 mL) was added 2M aq. NaOH (200 mg, 6 mmol) at 20° C. The mixture was stirred at 20° C. for 2 h, at which time, LCMS showed 61% starting material remained and 10% desired product was observed. The mixture was then stirred at 50° C. for 24 h. LCMS showed no starting material remained and 90% of the desired product was observed. The reaction mixture was acidified to pH=1 with 1N HCl, evaporated in vacuo to give a crude, which was then dissolved with DCM (20 mL), filtered to remove the inorganic salt. The DCM filtrate was dried over Na₂SO₄, filtered, and evaporated to give 5-(4-(1-(2-methyl-5-(4-methylpiperazin-1-yl)benzamido)cyclopropyl)naphthalen-2-yl)-1H-pyrrole-3-carboxylic acid (70 mg, 90.0%) as the white solid. LCMS (ESI): m/z=509.3, [M+H]⁺.

### Step 4: Preparation of N,N-dimethyl-5-(4-(1-(2-methyl-5-(4-methylpiperazin-1-yl)benzamido)cyclopropyl)naphthalen-2-yl)-1H-pyrrole-3-carboxamide

To a solution of 5-(4-(1-(2-methyl-5-(4-methylpiperazin-1-yl)benzamido)cyclopropyl)naphthalen-2-yl)-1H-pyrrole-3-carboxylic acid (90.0 mg, 0.18 mmol) in DMF (4.0 mL) were added HATU (101 mg, 0.265 mmol), DIEA (68.6 mg, 0.531 mmol), and Me₂NH·HCl (21.6 mg, 0.265 mmol) at 20° C. The mixture was then stirred at 20° C. for 4 h before being evaporated in vacuo to give a crude, which was purified by prep-HPLC to affords N,N-dimethyl-5-(4-(1-(2-methyl-5-(4-methylpiperazin-1-yl)benzamido)cyclopropyl)naphthalen-2-yl)-1H-pyrrole-3-carboxamide as a white solid after lyophilization. ¹H NMR (400 MHz, DMSO-d6) δ=11.90 (br s, 1H), 9.05 (s, 1H), 8.61 (br d, J=6.5 Hz, 1H), 8.08 (d, J=6.4 Hz, 2H), 7.97-7.76 (m, 1H), 7.57-7.45 (m, 2H), 7.31 (s, 1H), 7.02-6.88 (m, 2H), 6.83 (dd, J=2.5, 8.5 Hz, 1H), 6.57 (d, J=2.6 Hz, 1H), 3.22-2.93 (m, 10H), 2.44-2.32 (m, 4H), 2.19 (s, 3H), 1.93 (s, 3H), 1.40-1.26 (m, 4H). LCMS (ESI): m/z=536.4, [M+H]⁺.

### Example 278: rel-5-((1S,4S)-2,5-diazabicyclo[2.2.1]heptan-2-yl)-2-methyl-N-(1-(2-(1-methyl-1H-pyrazol-4-yl)quinolin-4-yl)cyclopropyl)benzamide, ENT-1

and

### Example 279

rel-5-((1R,4R)-2,5-diazabicyclo[2.2.1]heptan-2-yl)-2-methyl-N-(1-(2-(1-methyl-1H-pyrazol-4-yl)quinolin-4-yl)cyclopropyl)benzamide, ENT-2

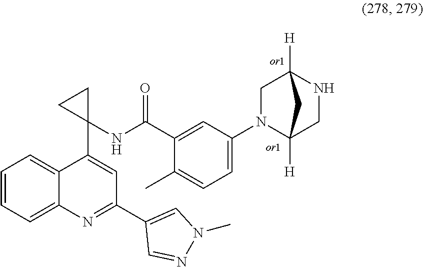 [SMILES: Cc1ccc(N2C[C@H]3C[C@@H]2CN3)cc1C(=O)NC1(c2cc(-c3cnn(C)c3)nc3ccccc23)CC1]

### Step 1: Preparation of tert-butyl 5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate

To a solution of 5-(5-(tert-butoxycarbonyl)-2,5-diazabicyclo[2.2.1]heptan-2-yl)-2-methylbenzoic acid (377 mg, 1.13 mmol) in DMF (20.0 mL) were added HATU (647 mg, 1.70 mmol), TEA (3.0 mL), and 1-(2-(1-methyl-1H-pyrazol-4-yl)quinolin-4-yl)cyclopropan-1-amine (300.0 mg, 1.13 mmol) at 20° C. The reaction mixture was stirred at 20° C. for 2 h. After that, the reaction mixture was partitioned between ethyl acetate and H₂O (50/50 mL). The organic layer was separated, and the aqueous layer was re-extracted with ethyl acetate (50 mL). The combined organic layer was washed with brine, dried with Na₂SO₄, evaporated in vacuo, and then purified by silica gel chromatography via Biotage (20 g silica column, PE:EA=1:0 to 0:1) to afford tert-butyl 5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate (333 mg, 50.7%) as a yellow solid. LCMS (ESI): m/z=579.2, [M+H]⁺.

### Step 2: SFC Separation

Two isomers of tert-butyl 5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate (300 mg, 0.518 mmol) were separated by SFC to afford rel-tert-butyl(1R,4R)-5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate, ENT-1 (132 mg, 44%, peak 1) and rel-tert-butyl(1R,4R)-5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate, ENT-2 (90 mg, 30%, peak 2) as two white solids.

### Sfc Condition:

Column: DAICEL CHIRALPAK IG (250 mm*30 mm, 10 μm);
Mobile phase: CO₂-EtOH (0.1% NH₃·H₂O); B %: 60%-60%,
Flow rate (mL/min): 80 mL/min.

### Step 3a: Boc Deprotection of rel-tert-butyl(1R,4R)-5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate, ENT-1

To a solution of rel-tert-butyl(1R,4R)-5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate, ENT-1 (132 mg, 0.228 mmol) in CH₂Cl₂ (20 mL) was added HCl/dioxane (5 mL, 4M) at 20° C. The reaction mixture was stirred at 20° C. for 1 h. After that, the reaction mixture was evaporated in vacuo to afford a crude, which was then dissolved in MeOH (8 mL). The mixture was basified with 3 mL NH₃·H₂O at 0° C. then was evaporated in vacuo to give a yellow solid. The solid was dissolved in 5 mL DMF and purified by prep-HPLC to afford rel-5-((1R,4R)-2,5-diazabicyclo[2.2.1]heptan-2-yl)-2-methyl-N-(1-(2-(1-methyl-1H-pyrazol-4-yl)quinolin-4-yl)cyclopropyl)benzamide, ENT-1 (36 mg, 33%) as a white solid after lyophilization. ¹H NMR (400 MHz, MeOD) δ ppm 8.56 (d, J=8.4 Hz, 1H), 8.41 (s, 1H), 8.27 (s, 1H), 8.11-8.05 (m, 2H), 7.76 (t, J=7.3 Hz, 1H), 7.61 (t, J=7.7 Hz, 1H), 6.95 (d, J=8.3 Hz, 1H), 6.54 (d, J=7.9 Hz, 1H), 6.31 (s, 1H), 4.27 (s, 1H), 4.03 (s, 3H), 3.75 (s, 1H), 3.49 (dd, J=2.1, 9.1 Hz, 1H), 2.95-2.87 (m, 3H), 1.99-1.90 (m, 4H), 1.76 (br d, J=9.9 Hz, 1H), 1.53-1.40 (m, 4H). LCMS (ESI): m/z=479.4, [M+H]⁺.

### Prep-HPLC Condition:

Column: Boston Prime C18 150*30 mm*5 μm;
Mobile phase: Water (NH₃·H₂O—NH₄HCO₃)-MeCN; B %: 26%-66%,
Flow rate (mL/min): 30 mL/min.

### Step 3b: Boc deprotection of rel-tert-butyl(1R,4R)-5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate, ENT-2

Using a similar procedure to Step 3a of Example 278 and Example 279 using rel-tert-butyl (1R,4R)-5-(4-methyl-3-((1-(2-(1-methyl-1H-pyrazol-4-yl)quinolin-4-yl)cyclopropyl)carbamoyl)phenyl)-2,5-diazabicyclo[2.2.1]heptane-2-carboxylate, ENT-2 (90 mg, 0.16 mmol) affords rel-5-((1R,4R)-2,5-diazabicyclo[2.2.1]heptan-2-yl)-2-methyl-N-(1-(2-(1-methyl-1H-pyrazol-4-yl)quinolin-4-yl)cyclopropyl)benzamide, ENT-2 (17 mg, 23%) as a white solid. ¹H NMR (400 MHz, MeOD) δ ppm 8.56 (d, J=7.7 Hz, 1H), 8.41 (s, 1H), 8.27 (s, 1H), 8.11-8.05 (m, 2H), 7.78-7.73 (m, 1H), 7.61 (t, J=7.7 Hz, 1H), 6.95 (d, J=8.3 Hz, 1H), 6.54 (d, J=8.6 Hz, 1H), 6.31 (s, 1H), 4.26 (s, 1H), 4.03 (s, 3H), 3.75 (s, 1H), 3.48 (dd, J=2.1, 9.2 Hz, 1H), 2.95-2.86 (m, 3H), 1.98 (s, 3H), 1.92 (br d, J=9.8 Hz, 1H), 1.76 (br d, J=9.5 Hz, 1H), 1.53-1.40 (m, 4H). LCMS (ESI): m/z=479.4, [M+H]⁺.

### Example 280: 2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-1

and

### Example 281: 2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-2

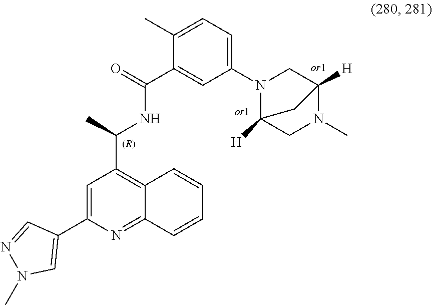

Two isomers of 2-methyl-N—((R)-1-(2-(1-methyl-1H-pyrazol-4-yl)quinolin-4-yl)ethyl)-5-(5-methyl-2,5-diazabicyclo[2.2.1]heptan-2-yl)benzamide (99 mg, 0.205 mmol) were separated by SFC to afford 2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-1 (38.32 mg, 38%) and 2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-2 (13.84 mg, 14%).

### SFC Separation Condition:

Column: DAICEL CHIRALPAK AD (250 mm*30 mm, 10 μm);
Mobile phase: CO₂-EtOH (0.1% NH₃·H₂O); B %: 35%-35%,
Flow rate: 110 mL/min.

2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-1. ¹H NMR (400 MHz, METHANOL-d₄) δ=8.21 (s, 1H), 8.16 (d, J=7.9 Hz, 1H), 8.05 (s, 1H), 7.95 (d, J=7.9 Hz, 1H), 7.75 (s, 1H), 7.66 (ddd, J=1.1, 7.0, 8.4 Hz, 1H), 7.51 (ddd, J=1.2, 7.0, 8.3 Hz, 1H), 6.95 (d, J=8.4 Hz, 1H), 6.51 (dd, J=2.6, 8.4 Hz, 1H), 6.47 (d, J=2.5 Hz, 1H), 5.90 (q, J=7.1 Hz, 1H), 4.16 (s, 1H), 3.89 (s, 3H), 3.42 (s, 1H), 3.33-3.27 (m, 1H), 3.16-3.11 (m, 1H), 2.72-2.59 (m, 2H), 2.25 (s, 3H), 2.11 (s, 3H), 1.95-1.84 (m, 1H), 1.84-1.76 (m, 1H), 1.62 (d, J=7.0 Hz, 3H). LCMS (ESI): m/z=481.4, [M+H]⁺.

and

2-methyl-N—((R)-1-(2-(1-methyl-1H-pyrazol-4-yl)quinolin-4-yl)ethyl)-5-((1R*,4R*)-5-methyl-2,5-diazabicyclo[2.2.1]heptan-2-yl)benzamide, ENT-2. ¹H NMR (400 MHz, METHANOL-d₄) δ=8.22 (s, 1H), 8.16 (d, J=8.3 Hz, 1H), 8.07-8.04 (m, 1H), 7.95 (d, J=8.0 Hz, 1H), 7.78-7.74 (m, 1H), 7.69-7.63 (m, 1H), 7.54-7.46 (m, 1H), 6.95 (d, J=8.4 Hz, 1H), 6.58-6.44 (m, 2H), 5.90 (q, J=7.0 Hz, 1H), 4.16 (s, 1H), 3.90 (s, 3H), 3.47-3.40 (m, 1H), 3.32-3.23 (m, 1H), 3.17-3.11 (m, 1H), 2.72-2.56 (m, 2H), 2.31-2.23 (m, 3H), 2.12 (s, 3H), 1.96-1.84 (m, 1H), 1.84-1.75 (m, 1H), 1.64-1.60 (m, 3H). LCMS (ESI): m/z=481.4, [M+H]⁺.

### Example 282: (R)-2-methyl-4-((((1-methyl-1H-imidazol-2-yl)methyl)thio)methyl)-N-(1-(2-(1-methyl-1H-pyrazol-4-yl)quinolin-4-yl)ethyl)benzamide

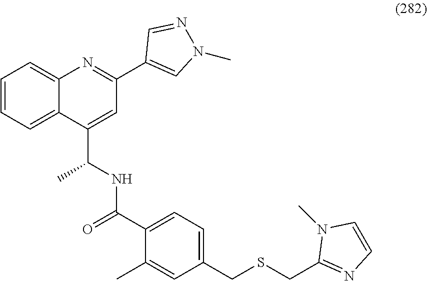 [SMILES: Cc1cc(CSCc2nccn2C)ccc1C(=O)N[C@H](C)c1cc(-c2cnn(C)c2)nc2ccccc12]

### Step 1: Preparation of S-((1-methyl-1H-imidazol-2-yl)methyl) ethanethioate

To a solution of 2-(chloromethyl)-1-methyl-1H-imidazole (2000 mg, 11.97 mmol) in DMF (59.9 mL) were added KSAc (1260 mg, 11.0 mmol) and Cs₂CO₃ (5850 mg, 18.0 mmol) at 20° C. The mixture was stirred at 20° C. for 16 h before being diluted with H₂O (40 mL) then extracted with ethyl acetate (100 mL×2). The combined organic layer was evaporated in vacuo to give a crude, which was purified by column chromatography (MeOH/DCM from 0% to 3%) to give S-((1-methyl-1H-imidazol-2-yl)methyl) ethanethioate (1000 mg, 49.1%) as a yellow solid. ¹H NMR (400 MHz, METHANOL-d₄) δ=7.00 (d, J=1.3 Hz, 1H), 6.84 (d, J=1.1 Hz, 1H), 4.26 (s, 2H), 3.66 (s, 3H), 2.37 (s, 3H). LCMS (ESI): m/z=171.0 [M+H]⁺.

### Step 2: Preparation of (1-methyl-1H-imidazol-2-yl)methanethiol

To a solution of S-((1-methyl-1H-imidazol-2-yl)methyl) ethanethioate (1000.0 mg, 5.874 mmol) in THF (9.8 mL) and MeOH (9.8 mL) was added 10% aq. NaOH solution (2.35 mL). The reaction solution was stirred at 25° C. for 3 h then evaporated in vacuo to give (1-methyl-1H-imidazol-2-yl)methanethiol as a yellow solid, which was used in the next step without further purification. LCMS (ESI): m/z=129.9 [M+H]⁺.

### Step 3: Preparation of methyl 2-methyl-4-((((1-methyl-1H-imidazol-2-yl)methyl)thio)methyl)benzoate

To a solution of (1-methyl-1H-imidazol-2-yl)methanethiol (300 mg, 2.34 mmol) in MeCN (33.4 mL) were added K₂CO₃ (647 mg, 4.68 mmol) and methyl 4-(chloromethyl)-2-methylbenzoate (465 mg, 2.34 mmol) at 20° C. The mixture was then stirred at 60° C. for 16 h before being evaporated in vacuo to give a crude, which was then dissolved in H₂O (20 mL) and extracted with ethyl acetate (30 mL×3). The combined organic layer was dried over Na₂SO₄, evaporated in vacuo to remove organic solvent and purified by column chromatography (MeOH/DCM from 0% to 3%) to give methyl 2-methyl-4-((((1-methyl-1H-imidazol-2-yl)methyl)thio)methyl)benzoate (370 mg, 54.4%) as a yellow solid. ¹H NMR (400 MHz, CHLOROFORM-d) δ=7.84 (d, J=7.9 Hz, 1H), 7.24-7.18 (m, 2H), 6.99 (d, J=1.3 Hz, 1H), 6.82 (d, J=1.1 Hz, 1H), 3.94 (s, 2H), 3.88 (s, 3H), 3.81 (s, 2H), 3.65 (s, 3H), 2.58 (s, 3H). LCMS (ESI): m/z=290.9 [M+H]⁺.

### Step 4: Preparation of 2-methyl-4-((((1-methyl-1H-imidazol-2-yl)methyl)thio)methyl)benzoic acid

To a solution of methyl 2-methyl-4-((((1-methyl-1H-imidazol-2-yl)methyl)thio)methyl)benzoate (250.0 mg, 0.861 mmol) in THF (6.0 mL), MeOH (1.0 mL), and H₂O (2.0 mL) was added LiOH·H₂O (108 mg, 2.58 mmol) at 20° C. The reaction was stirred at 40° C. for 16 h before being neutralized with 1N HCl to pH=6-7 and evaporated in vacuo to give 2-methyl-4-((((1-methyl-1H-imidazol-2-yl)methyl)thio)methyl)benzoic acid (420 mg) as a yellow solid, which was used in the next step without further purification. LCMS (ESI): m/z=276.9 [M+H]⁺.

### Step 5: Preparation of (R)-2-methyl-4-((((1-methyl-1H-imidazol-2-yl)methyl)thio)methyl)-N-(1-(2-(1-methyl-1H-pyrazol-4-yl)quinolin-4-yl)ethyl)benzamide

To a solution of 2-methyl-4-((((1-methyl-1H-imidazol-2-yl)methyl)thio)methyl)benzoic acid (400 mg, 0.72 mmol) in DMF (7.24 mL) were added HATU (413 mg, 1.09 mmol), DIEA (281 mg, 2.17 mmol), and (R)-1-(2-(1-methyl-1H-pyrazol-4-yl)quinolin-4-yl)ethan-1-amine (183 mg, 0.724 mmol) at 20° C. The mixture was then stirred at 20° C. for 3 h before being purified by prep-HPLC to afford (R)-2-methyl-4-((((1-methyl-1H-imidazol-2-yl)methyl)thio)methyl)-N-(1-(2-(1-methyl-1H-pyrazol-4-yl)quinolin-4-yl)ethyl)benzamide (48.4 mg, 12%) as a yellow solid. ¹H NMR (400 MHz, METHANOL-d₄) δ=8.33 (s, 1H), 8.26 (br d, J=8.1 Hz, 2H), 8.16 (s, 1H), 8.06 (d, J=8.1 Hz, 1H), 7.84 (s, 1H), 7.79-7.74 (m, 1H), 7.64-7.57 (m, 1H), 7.31 (d, J=7.8 Hz, 1H), 7.20-7.14 (m, 2H), 7.09 (d, J=1.1 Hz, 1H), 6.95 (d, J=1.3 Hz, 1H), 6.01 (q, J=6.9 Hz, 1H), 4.00 (s, 3H), 3.84 (s, 2H), 3.72 (s, 2H), 3.65 (s, 3H), 2.32 (s, 3H), 1.73 (d, J=7.0 Hz, 3H). LCMS (ESI): m/z=511.4 [M+H]⁺.

### Prep-HPLC Condition:

Column: Boston Prime C18 150*30 mm;
Mobile phase: Water (FA)-MeCN; B %: 0%-30%,
Flow Rate (mL/min): 30 mL/min.

### Example 283: 2-methyl-4-((((1-methyl-1H-imidazol-2-yl)methyl)sulfinyl)methyl)-N—((R)-1-(2-(1-methyl-1H-pyrazol-4-yl)quinolin-4-yl)ethyl)benzamide

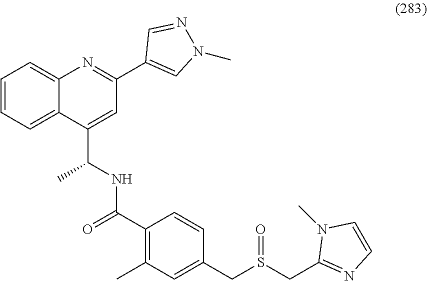 [SMILES: Cc1cc(CS(=O)Cc2nccn2C)ccc1C(=O)N[C@H](C)c1cc(-c2cnn(C)c2)nc2ccccc12]

### Step 1: Preparation of methyl 2-methyl-4-((((1-methyl-1H-imidazol-2-yl)methyl)sulfinyl)methyl)benzoate

To a solution of methyl 2-methyl-4-((((1-methyl-1H-imidazol-2-yl)methyl)thio)methyl)benzoate (100 mg, 0.344 mmol) in DCM (4.92 mL) was slowly added m-CPBA (69.9 mg, 0.344 mmol) in several portions at 20° C. After adding m-CPBA, the mixture was stirred at 20° C. for 4 h before being quenched by an aqueous solution of Na₂S₂O₃ (5 mL). The mixture was then extracted with DCM (10 mL×2). The combined organic layer was evaporated in vacuo to give a crude, which was purified by prep-HPLC to afford methyl 2-methyl-4-((((1-methyl-1H-imidazol-2-yl)methyl)sulfinyl)methyl)benzoate (66 mg, 63.0%) as a yellow solid. ¹H NMR (400 MHz, CHLOROFORM-d) δ=7.92 (d, J=7.7 Hz, 1H), 7.25 (s, 2H), 7.19 (s, 1H), 7.01 (s, 1H), 4.58 (d, J=14.1 Hz, 1H), 4.34 (d, J=13.1 Hz, 1H), 4.23 (br d, J=14.1 Hz, 1H), 3.93-3.91 (m, 1H), 3.90 (s, 3H), 3.78 (s, 3H), 2.61 (s, 3H). LCMS (ESI): m/z=306.9 [M+H]⁺.

### Prep-HPLC Condition:

Column: Boston Prime C18 150×30 mm;
Mobile phase: Water (FA)-MeCN; B %: 0%-25%,
Flow rate (mL/min): 30 mL/min.

### Step 2: Preparation of 2-methyl-4-((((1-methyl-1H-imidazol-2-yl)methyl)sulfinyl)methyl)benzoic acid

To a solution of methyl 2-methyl-4-((((1-methyl-1H-imidazol-2-yl)methyl)sulfinyl)methyl)benzoate (66 mg, 0.22 mmol) in THF (1.5 mL), MeOH (0.25 mL), and H₂O (0.5 mL) was added LiOH·H₂O (27.1 mg, 0.646 mmol) at 20° C. The reaction was then stirred at 40° C. for 16 h before being neutralized by 1 N HCl to pH=6-7 and evaporated in vacuo to give 2-methyl-4-((((1-methyl-1H-imidazol-2-yl)methyl)sulfinyl)methyl)benzoic acid (63 mg) as a yellow solid, which was used in the next step without further purification. LCMS (ESI): m/z=292.9 [M+H]⁺.

### Step 3: Preparation of 2-methyl-4-((((1-methyl-1H-imidazol-2-yl)methyl)sulfinyl)methyl)-N—((R)-1-(2-(1-methyl-1H-pyrazol-4-yl)quinolin-4-yl)ethyl)benzamide

To a solution of 2-methyl-4-((((1-methyl-1H-imidazol-2-yl)methyl)sulfinyl)methyl)benzoic acid (63 mg, 0.22 mmol) in DMF (2.15 mL) were added HATU (123 mg, 0.323 mmol), DIEA (83.6 mg, 0.646 mmol) and (R)-1-(2-(1-methyl-1H-pyrazol-4-yl)quinolin-4-yl)ethan-1-amine (54.4 mg, 0.215 mmol) at 20° C. The mixture was stirred at 20° C. for 3 h then purified by prep-HPLC to give 2-methyl-4-((((1-methyl-1H-imidazol-2-yl)methyl)sulfinyl)methyl)-N—((R)-1-(2-(1-methyl-1H-pyrazol-4-yl)quinolin-4-yl)ethyl)benzamide (41.8 mg, 37%) as a white solid. ¹H NMR (400 MHz, METHANOL-d₄) δ=8.32 (s, 1H), 8.27 (d, J=8.1 Hz, 1H), 8.16 (s, 1H), 8.06 (d, J=8.6 Hz, 1H), 7.85 (s, 1H), 7.76 (t, J=7.6 Hz, 1H), 7.65-7.58 (m, 1H), 7.40 (d, J=8.1 Hz, 1H), 7.30-7.26 (m, 2H), 7.15 (s, 1H), 7.01 (s, 1H), 6.03 (q, J=7.0 Hz, 1H), 4.37 (d, J=14.2 Hz, 1H), 4.28 (d, J=13.0 Hz, 1H), 4.21 (d, J=14.3 Hz, 1H), 4.06 (d, J=13.1 Hz, 1H), 4.00 (s, 3H), 3.72 (s, 3H), 2.36 (s, 3H), 1.74 (d, J=7.0 Hz, 3H). LCMS (ESI): m/z=527.4 [M+H]⁺.

### Prep-HPLC Condition:

Column: Boston Prime C18 150×30 mm;
Mobile phase: Water (NH₃·H₂O—NH₄HCO₃)-MeCN; B %: 13%-43%,
Flow rate (mL/min): 30 mL/min.

### Example 321: 4-methyl-N—((R)-1-(2-(1-methyl-1H-pyrazol-4-yl)quinolin-4-yl)ethyl)-6-(1-(thiazol-4-ylmethoxy)ethyl)nicotinamide

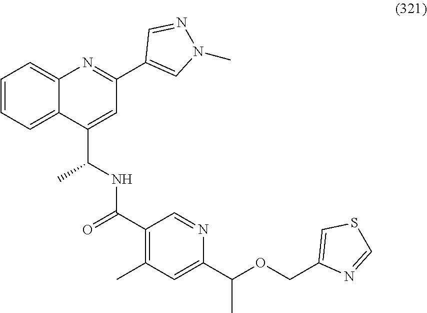 [SMILES: Cc1cc(C(C)OCc2cscn2)ncc1C(=O)N[C@H](C)c1cc(-c2cnn(C)c2)nc2ccccc12]

### Step 1: Preparation of 1-(5-bromo-4-methylpyridin-2-yl)ethan-1-ol

To a solution of 5-bromo-4-methylpicolinaldehyde (500 mg, 2.50 mmol) in THF (5 mL) at 0° C. was added MeMgBr (1 mL, 3.00 mmol, 3M) dropwise. After that, the reaction was stirred at 0° C. for 1 h. The mixture was then quenched with sat. aq. NH₄Cl (10 mL), then extracted with ethyl acetate (10 mL×3). The combined organic layer was dried over Na₂SO₄ and evaporated in vacuo to give 1-(5-bromo-4-methylpyridin-2-yl)ethan-1-ol (500 mg, 92.6%) as a yellow oil. ¹H NMR (400 MHz, CHLOROFORM-d) δ=8.58 (s, 1H), 7.28 (s, 1H), 4.86 (q, J=6.5 Hz, 1H), 2.44 (s, 3H), 1.51 (d, J=6.6 Hz, 3H). LCMS (ESI): m/z=218.0, [M+H]⁺.

### Step 2: Preparation of ethyl 6-(1-hydroxyethyl)-4-methylnicotinate

To a solution of 1-(5-bromo-4-methylpyridin-2-yl)ethan-1-ol (500.0 mg, 2.31 mmol) in EtOH (30.0 mL) were added TEA (702 mg, 6.94 mmol) and Pd(dppf)Cl₂ (254 mg, 0.347 mmol) at 20° C. The mixture was degassed with N₂ before being stirred under CO (50 psi) at 80° C. for 12 h. After that, the reaction mixture was filtered and evaporated in vacuo to give a crude, which was purified using a Biotage (12 g silica gel, eluted with PE/EA from 0% to 50%) to give ethyl 6-(1-hydroxyethyl)-4-methylnicotinate (390 mg) as an oil. ¹H NMR (400 MHz, CHLOROFORM-d) δ=9.04 (s, 2H), 7.28 (s, 1H), 4.92 (q, J=6.5 Hz, 1H), 4.40 (q, J=7.2 Hz, 2H), 2.67 (s, 3H), 1.53 (d, J=6.6 Hz, 3H), 1.43 (t, J=7.2 Hz, 3H). LCMS (ESI): m/z=210.1, [M+H]⁺.

### Step 3: Preparation of ethyl 4-methyl-6-(1-(thiazol-4-ylmethoxy)ethyl)nicotinate

To a solution of ethyl 6-(1-hydroxyethyl)-4-methylnicotinate (100.0 mg, 0.478 mmol) in DMF (2.39 mL) were added 4-(chloromethyl)thiazole (63.8 mg, 0.48 mmol) and NaH (28.7 mg, 0.717 mmol) at 20° C. The reaction was stirred for 3 h before being quenched by H₂O (10 mL) and extracted with ethyl acetate (10 mL×3). The combined organic layer was dried over Na₂SO₄ and evaporated in vacuo to give a crude, which was purified using a Biotage (4 g silica gel, eluted with PE/EA from 0% to 100%) to give ethyl 4-methyl-6-(1-(thiazol-4-ylmethoxy)ethyl)nicotinate (45 mg, 31%) as a solid. LCMS (ESI): m/z=307.1, [M+H]⁺.

### Step 4: Preparation of 4-methyl-6-(1-(thiazol-4-ylmethoxy)ethyl)nicotinic acid

To a solution of ethyl 4-methyl-6-(1-(thiazol-4-ylmethoxy)ethyl)nicotinate (45 mg, 0.15 mmol) in THF (1.0 mL) and H₂O (0.5 mL) was added LiOH·H₂O (10.6 mg, 0.441 mmol). The reaction was stirred at 25° C. for 8 h. After that, the mixture was adjusted to pH=5-6 by 1 N HCl solution and then extracted with ethyl acetate (5 mL×3). LCMS showed the desired product stayed in the aqueous layer. The aqueous layer was concentrated to give a crude product, which was purified by flash chromatography (4 g silica gel, eluted with PE/EA from 0% to 100%) to give 4-methyl-6-(1-(thiazol-4-ylmethoxy)ethyl)nicotinic acid (25 mg, 61%) as a solid. LCMS (ESI): m/z=279.0, [M+H]⁺.

### Step 5: Preparation of 4-methyl-N—((R)-1-(2-(1-methyl-1H-pyrazol-4-yl)quinolin-4-yl)ethyl)-6-(1-(thiazol-4-ylmethoxy)ethyl)nicotinamide

To a solution of 4-methyl-6-(1-(thiazol-4-ylmethoxy)ethyl)nicotinic acid (25 mg, 0.090 mmol) in DMF (1.5 mL) were added (R)-1-(2-(1-methyl-1H-pyrazol-4-yl)quinolin-4-yl)ethan-1-amine (22.7 mg, 0.0898 mmol), DIEA (34.8 mg, 0.269 mmol), and HATU (51.2 mg, 0.135 mmol) at 20° C. The reaction was stirred at 20° C. for 2 h. After that the mixture was purified by prep-HPLC to give 4-methyl-N—((R)-1-(2-(1-methyl-1H-pyrazol-4-yl)quinolin-4-yl)ethyl)-6-(1-(thiazol-4-ylmethoxy)ethyl)nicotinamide (15 mg, 33%). ¹H NMR (400 MHz, DMSO-d₆) δ=9.17 (d, J=7.5 Hz, 1H), 9.08 (d, J=2.0 Hz, 1H), 8.52 (s, 1H), 8.44 (s, 1H), 8.23 (d, J=8.8 Hz, 1H), 8.11 (d, J=1.3 Hz, 1H), 7.98 (d, J=8.4 Hz, 1H), 7.86 (s, 1H), 7.77-7.72 (m, 1H), 7.64 (s, 1H), 7.59 (t, J=7.1 Hz, 1H), 7.41 (s, 1H), 5.91-5.88 (m, 1H), 4.66 (m, 1H), 4.61-4.52 (m, 2H), 3.94 (s, 3H), 2.34 (d, J=4.6 Hz, 3H), 1.63 (d, J=6.8 Hz, 3H), 1.42 (d, J=6.4 Hz, 3H). LCMS (ESI): m/z=513.3, [M+H]⁺.

### Prep-HPLC Condition:

Column: C18-1 150*40 mm*5 μm
Mobile phase: water (ammonium hydroxide)-MeCN; B %: 30%-50%,
Flow rate (mL/min): 35 mL/min.

### Example 284-304 and 322-323

| Ex. | Structure; | Analogous method, | |
|---|---|---|---|
| # | Compound Name | reactants | Characterizing Data |
| Ex. 284 | 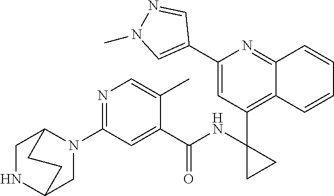 [SMILES: Cc1cnc(N2CC3CCC2CN3)cc1C(=O)NC1(c2cc(-c3cnn(C)c3)nc3ccccc23)CC1] | Example 251 using 2-(5- (tert-butoxycarbonyl)- 2,5-diazabicyclo [2.2.2]octan-2-yl)-5- methylisonicotinic acid and 1-(2-(1-methyl-1H- pyrazol-4-yl)quinolin-4- yl)cyclopropan-1-amine | ¹H NMR (400 MHz, METHANOL-d₄) δ = 8.53 (d, J = 8.0 Hz, 1H), 8.41 (s, 1H), 8.27 (s, 1H), 8.13-8.04 (m, 2H), 7.83 (s, 1H), 7.79-7.72 (m, 1H), 7.61 (t, J = 7.3 Hz, 1H), 6.28 (s, 1H), 4.42 (br s, 1H), 4.03 (s, 3H), 3.58 (br d, J = 10.8 Hz, 1H), 3.42 (dd, J = 1.6, 10.6 Hz, 1H), 3.23 (br s, 1H), 3.20-3.09 (m, 2H), 2.06-1.74 (m, 7H), 1.55- 1.42 (m, 4H). LCMS (ESI): m/z = 494.4, [M + H]⁺. |
| | 2-(2,5-diazabicyclo[2.2.2] | | |
| | octan-2-yl)-5-methyl-N-(1- | | |
| | (2-(1-methyl-1H-pyrazol-4- | | |
| | yl)quinolin-4-yl)cyclopropyl) | | |
| | isonicotinamide | | |
| Ex. 285 | 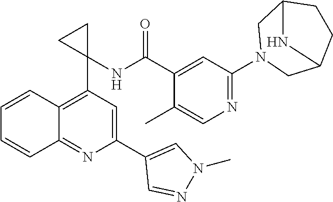 [SMILES: Cc1cnc(N2CC3CCC(C2)N3)cc1C(=O)NC1(c2cc(-c3cnn(C)c3)nc3ccccc23)CC1] | Example 251 using 2-(8- (tert-butoxycarbonyl)- 3,8-diazabicyclo [3.2.1]octan-3-yl)-5- methylisonicotinic acid and 1-(2-(1-methyl-1H- pyrazol-4-yl)quinolin-4- yl)cyclopropan-1-amine | ¹H NMR (400 MHz, METHANOL-d₄) δ = 8.53 (d, J = 8.4 Hz, 1H), 8.41 (s, 1H), 8.27 (s, 1H), 8.13-8.04 (m, 2H), 7.87 (s, 1H), 7.80-7.72 (m, 1H), 7.61 (t, J = 7.7 Hz, 1H), 6.43 (s, 1H), 4.03 (s, 3H), 3.75 (br d, J = 12.0 Hz, 2H), 3.60 (br s, 2H), 2.92 (br d, J = 10.6 Hz, 2H), 1.90 (s, 3H), 1.85-1.70 (m, 4H), 1.55- 1.48 (m, 2H), 1.48-1.42 (m, 2H). LCMS (ESI): m/z = 494.4, [M + H]⁺. |
| | 2-(3,8-diazabicyclo | | |
| | [3.2.1]octan-3-yl)-5-methyl- | | |
| | N-(1-(2-(1-methyl-1H- | | |
| | pyrazol-4-yl)quinolin-4- | | |
| | yl) cyclopropyl) | | |
| | isonicotinamide | | |
| Ex. 286 | 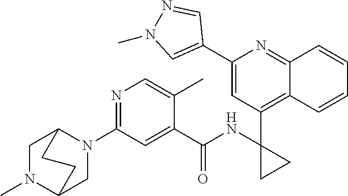 [SMILES: Cc1cnc(N2CC3CCC2CN3C)cc1C(=O)NC1(c2cc(-c3cnn(C)c3)nc3ccccc23)CC1] 5-methyl-N-(1-(2-(1-methyl- 1H-pyrazol-4-yl)quinolin-4- yl)cyclopropyl)-2-(5-methyl- 2,5-diazabicyclo | Example 252 using 2- (2,5-diazabicyclo[2.2.2] octan-2-yl)-5-methyl-N- (1-(2-(1-methyl-1H- pyrazol-4-yl)quinolin-4- yl) cyclopropyl) isonicotinamide | ¹H NMR (400 MHz, METHANOL-d₄) δ = 8.53 (dd, J = 0.8, 8.4 Hz, 1H), 8.41 (s, 1H), 8.27 (s, 1H), 8.11-8.05 (m, 2H), 7.82 (s, 1H), 7.76 (ddd, J = 1.3, 7.0, 8.4 Hz, 1H), 7.61 (ddd, J = 1.1, 7.0, 8.3 Hz, 1H), 6.28 (s, 1H), 4.35 (br s, 1H), 4.03 (s, 3H), 3.77-3.68 (m, 1H), 3.25 (dd, J = 1.7, 10.9 Hz, 1H), 2.99- 2.82 (m, 3H), 2.43 (s, 3H), 2.15 (br s, 1H), 1.89 (s, 3H), 1.87- 1.75 (m, 2H), 1.72-1.60 (m, 1H), 1.55-1.48 (m, 2H), 1.48-1.42 (m, 2H). LCMS (ESI): m/z = 508.4, [M + H]⁺. |
| | [2.2.2]octan-2- | | |
| | yl)isonicotinamide | | |
| Ex. 287 | 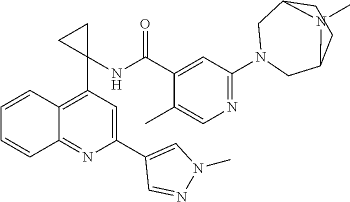 [SMILES: Cc1cnc(N2CC3CCC(C2)N3C)cc1C(=O)NC1(c2cc(-c3cnn(C)c3)nc3ccccc23)CC1] 5-methyl-N-(1-(2-(1-methyl- 1H-pyrazol-4-yl)quinolin-4- yl)cyclopropyl)-2-(8-methyl- | Example 252 using 2- (3,8-diazabicyclo[3.2.1] octan-3-yl)-5-methyl-N- (1-(2-(1-methyl-1H- pyrazol-4-yl)quinolin-4- yl)cyclopropyl) isonicotinamide | ¹H NMR (400 MHz, METHANOL-d₄) δ = 8.52 (dd, J = 0.8, 8.4 Hz, 1H), 8.41 (s, 1H), 8.26 (d, J = 0.6 Hz, 1H), 8.11- 8.05 (m, 2H), 7.86 (s, 1H), 7.76 (ddd, J = 1.3, 7.0, 8.4 Hz, 1H), 7.60 (ddd, J = 1.2, 6.9, 8.3 Hz, 1H), 6.42 (s, 1H), 4.03 (s, 3H), 3.69 (dd, J = 2.1, 12.0 Hz, 2H), 3.29 (br d, J = 1.6 Hz, 2H), 3.04-2.93 (m, 2H), 2.34 (s, 3H), 2.11-2.01 (m, 2H), 1.89 (s, 3H), 1.66 (d, J = 7.8 Hz, 2H), 1.56- 1.49 (m, 2H), 1.48-1.41 (m, 2H). LCMS (ESI): m/z = 508.5, [M + H]⁺. |
| | 3,8-diazabicyclo [3.2.1] | | |
| | octan-3-yl)isonicotinamide | | |
| Ex. 288 | 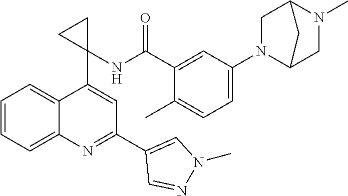 [SMILES: Cc1ccc(N2CC3CC2CN3C)cc1C(=O)NC1(c2cc(-c3cnn(C)c3)nc3ccccc23)CC1] 2-methyl-N-(1-(2-(1-methyl- 1H-pyrazol-4-yl)quinolin-4- yl)cyclopropyl)-4-(5-methyl- 2,5-diazabicyclo[2.2.1] heptan-2-yl)benzamide | Example 251 using 5-(5- (tert-butoxycarbonyl)- 2,5-diazabicyclo[2.2.1] heptan-2-yl)-2- methylbenzoic acid and 1-(2-(1-methyl-1H- pyrazol-4-yl)quinolin-4- yl)cyclopropan-1-amine; then Example 252 | ¹H NMR (400 MHz, METHANOL-d₄) δ ppm 8.541 (d, J = 7.82 Hz, 1H), 8.390 (s, 1H), 8.248 (s, 1H), 8.026-8.090 (m, 2H), 7.701-7.767 (m, 1H), 7.598 (br dd, J = 8.25, 1.16 Hz, 1H), 6.932 (d, J = 8.44 Hz, 1H), 6.521 (dd, J = 8.31, 2.57 Hz, 1H), 6.287 (d, J = 2.57 Hz, 1H), 4.159 (s, 1H), 4.006 (s, 3H), 3.472 (s, 1H), 3.291 (d, J = 2.32 Hz, 1H), 3.144 (d, J = 9.17 Hz, 1H), 2.704-2.755 (m, 1H), 2.600-2.658 (m, 1H), 2.317 (s, 3H), 1.952 (s, 4H), 1.847 (s, 1H), 1.480 (br d, J = 2.69 Hz, 2H), 1.369-1.442 (m, 2H), 1.027 (s, 1H). LCMS (ESI): m/z = 493.4, [M + H]⁺. |
| Ex. 289 | 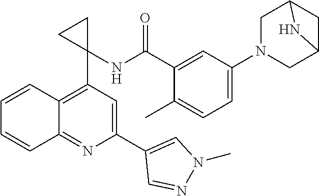 [SMILES: Cc1ccc(N2CC3CC(C2)N3)cc1C(=O)NC1(c2cc(-c3cnn(C)c3)nc3ccccc23)CC1] 5-(3,6- diazabicyclo[3.1.1]heptan- 3-yl)-2-methyl-N-(1-(2-(1- | Example 251 using 1-(2- (1-methyl-1H-pyrazol-4- yl)quinolin-4-yl) cyclopropan-1-amine and 5-(6-(tert- butoxycarbonyl)-3,6- diazabicyclo[3.1.1] heptan-3-yl)-2- methylbenzoic acid | ¹H NMR (400 MHz, METHANOL-d₄) δ = 8.58 (d, J = 7.8 Hz, 1H), 8.42 (s, 1H), 8.27 (s, 1H), 8.10 (s, 1H), 8.07 (d, J = 8.1 Hz, 1H), 7.76 (t, J = 7.3 Hz, 1H), 7.61 (t, J = 7.2 Hz, 1H), 7.01 (d, J = 8.5 Hz, 1H), 6.69 (dd, J = 2.7, 8.4 Hz, 1H), 6.47 (d, J = 2.5 Hz, 1H), 4.03 (s, 3H), 3.83 (br d, J = 5.9 Hz, 2H), 3.52-3.39 (m, 4H), 2.75-2.67 (m, 1H), 2.01 (s, 3H), 1.63 (d, J = 8.9 Hz, 1H), 1.55-1.40 (m, 4H). LCMS (ESI): m/z = 479.4, [M + H]⁺. |
| | methyl-1H-pyrazol-4- | | |
| | yl)quinolin-4-yl)cyclopropyl) | | |
| | benzamide | | |
| Ex. 290 | 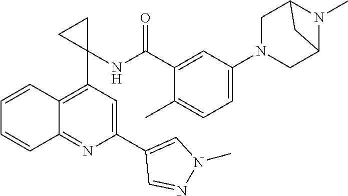 [SMILES: Cc1ccc(N2CC3CC(C2)N3C)cc1C(=O)NC1(c2cc(-c3cnn(C)c3)nc3ccccc23)CC1] 2-methyl-N-(1-(2-(1-methyl- 1H-pyrazol-4-yl)quinolin-4- yl)cyclopropyl)-5-(6-methyl- 3,6-diazabicyclo[3.1.1] | Example 252 using 5- (3,6-diazabicyclo[3.1.1] heptan-3-yl)-2-methyl-N- (1-(2-(1-methyl-1H- pyrazol-4-yl)quinolin-4- yl)cyclopropyl) benzamide | ¹H NMR (400 MHz, METHANOL-d₄) δ = 8.58 (d, J = 8.3 Hz, 1H), 8.42 (s, 1H), 8.27 (s, 1H), 8.10 (s, 1H), 8.07 (d, J = 8.4 Hz, 1H), 7.76 (t, J = 7.3 Hz, 1H), 7.61 (t, J = 7.3 1H), 7.02 (d, J = 8.6 Hz, 1H), 6.71 (dd, J = 2.6, 8.5 Hz, 1H), 6.50 (d, J = 2.6 Hz, 1H), 4.03 (s, 3H), 3.67 (br d, J = 6.0 Hz, 2H), 3.50-3.43 (m, 2H), 3.30 (br s, 2H), 2.58 (br s, 1H), 2.17-2.03 (m, 3H), 2.01 (s, 3H), 1.63 (br d, J = 8.4 Hz, 1H), 1.54-1.41 (m, 4H). LCMS (ESI): m/z = 493.4, [M + H]⁺. |
| | heptan-3-yl)benzamide | | |
| Ex. 291 | 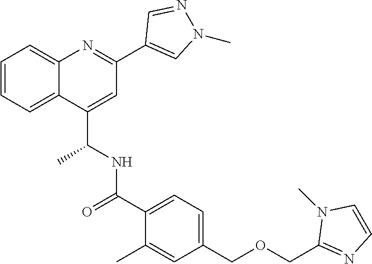 [SMILES: Cc1cc(COCc2nccn2C)ccc1C(=O)N[C@H](C)c1cc(-c2cnn(C)c2)nc2ccccc12] | Example 274 using (1- methyl-1H-imidazol-2- yl) methanol in Step 4 instead of thiazol-4- ylmethanol | ¹H NMR (400 MHz, DMSO-d₆) δ = 8.93 (d, J = 7.8 Hz, 1H), 8.41 (s, 1H), 8.22 (d, J = 8.3 Hz, 1H), 8.09 (s, 1H), 7.97 (d, J = 8.4 Hz, 1H), 7.85 (s, 1H), 7.73 (t, J = 7.6 Hz, 1H), 7.57 (t, J = 7.6 Hz, 1H), 7.36 (d, J = 7.6 Hz, 1H), 7.24-7.17 (m, 2H), 7.14 (s, 1H), 6.81 (s, 1H), 5.88 (br t, J = 7.3 Hz, 1H), 4.53 (s, 2H), 4.47 (s, 2H), 3.94 (s, 3H), 3.64 (s, 3H), 2.28 (s, 3H), 1.61 (d, J = 7.0 Hz, 3H). LCMS (ESI): m/z = 495.3, [M + H]⁺. |
| | (R)-2-methyl-4-(((1-methyl- | | |
| | 1H-imidazol-2-yl)methoxy) | | |
| | methyl)-N-(1-(2-(1-methyl- | | |
| | 1H-pyrazol-4-yl)quinolin-4- | | |
| | yl)ethyl) benzamide | | |
| Ex. 292 | 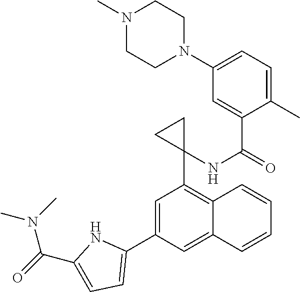 | Example 252 using N,N- dimethyl-5-(4-(1-(2- methyl-5-(piperazin-1- yl)benzamido) cyclopropyl)naphthalen- 2-yl)-1H-pyrrole-2- carboxamide as a reactant and NaBH(OAc)3 as the reducing agent. | ¹H NMR (400 MHz, DMSO-d6) δ = 11.89 (br s, 1H), 9.04 (br s, 1H), 8.62 (br s, 1H), 8.27 (br s, 1H), 8.20 (br s, 1H), 7.88 (br s, 1H), 7.51 (br d, J = 3.1 Hz, 2H), 6.96 (br d, J = 7.9 Hz, 1H), 6.84 (br s, 1H), 6.74 (br s, 1H), 6.68 (br s, 1H), 6.57 (br s, 1H), 3.16 (br s, 3H), 3.00 (br s, 3H), 2.56- 2.53 (m, 4H), 2.41 (br s, 4H), 2.21 (br s, 3H), 1.94 (br s, 3H), 1.35 (br s, 4H). LCMS (ESI): m/z = 536.4, [M + H]⁺. |
| | N,N-dimethyl-5-(4-(1-(2- | | |
| | methyl-5-(4-methylpiperazin- | | |
| | 1-yl)benzamido) | | |
| | cyclopropyl)naphthalen-2- | | |
| | yl)-1H-pyrrole-2- | | |
| | carboxamide | | |
| Ex. 293 | 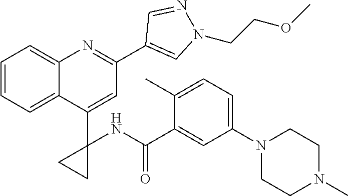 [SMILES: COCCn1cc(-c2cc(C3(NC(=O)c4cc(N5CCN(C)CC5)ccc4C)CC3)c3ccccc3n2)cn1] N-(1-(2-(1-(2- methoxyethyl)-1H-pyrazol- | Example 275 using (1- (2-methoxyethyl)-1H- pyrazol-4-yl)boronic acid instead of (3-methyl-1H- pyrazol-5-yl)boronic acid in Step 1 | ¹H NMR (400 MHz, MeOD) δ ppm 8.56 (d, J = 7.6 Hz, 1H), 8.46 (s, 1H), 8.30 (s, 1H), 8.10 (s, 1H), 8.08 (d, J = 8.6 Hz, 1H), 7.79-7.73 (m, 1H), 7.61 (t, J = 7.2 Hz, 1H), 7.03 (d, J = 8.6 Hz, 1H), 6.90 (d, J = 7.9 Hz, 1H), 6.69 (s, 1H), 4.43 (t, J = 5.2 Hz, 2H), 3.83 (t, J = 5.1 Hz, 2H), 3.38 (s, 3H), 3.16-3.05 (m, 4H), 2.64-2.51 (m, 4H), 2.34 (s, 3H), 2.01 (s, 3H), 1.54-1.40 (m, 4H). LCMS (ESI): m/z = 525.0, [M + H]⁺. |
| | 4-yl)quinolin-4- | | |
| | yl)cyclopropyl)-2-methyl-5- | | |
| | (4-methylpiperazin-1- | | |
| | yl)benzamide | | |
| Ex. 294 | 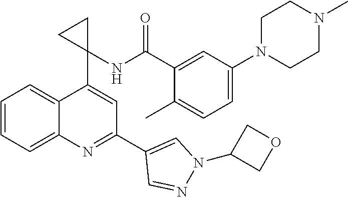 [SMILES: Cc1ccc(N2CCN(C)CC2)cc1C(=O)NC1(c2cc(-c3cnn(C4COC4)c3)nc3ccccc23)CC1] 2-methyl-5-(4- methylpiperazin-1-yl)-N-(1- | Example 275 using (1- (oxetan-3-yl)-1H- pyrazol-4-yl)boronic acid instead of (3-methyl-1H- pyrazol-5-yl)boronic acid in Step 1 | ¹H NMR (400 MHz, MeOD) δ ppm 8.61 (s, 1H), 8.57 (d, J = 8.0 Hz, 1H), 8.40 (s, 1H), 8.13 (s, 1H), 8.08 (d, J = 8.1 Hz, 1H), 7.76 (t, J = 7.3 Hz, 1H), 7.61 (t, J = 7.2 Hz, 1H), 7.03 (d, J = 8.0 Hz, 1H), 6.90 (d, J = 7.8 Hz, 1H), 6.69 (s, 1H), 5.70 (quin, J = 6.8 Hz, 1H), 5.13 (d, J = 6.8 Hz, 4H), 3.15-3.05 (m, 4H), 2.64-2.51 (m, 4H), 2.34 (s, 3H), 2.00 (s, 3H), 1.54-1.41 (m, 4H). LCMS (ESI): m/z = 523.3, [M + H]⁺. |
| | (2-(1-(oxetan-3-yl)-1H- | | |
| | pyrazol-4-yl)quinolin-4- | | |
| | yl)cyclopropyl)benzamide | | |
| Ex. 295 | 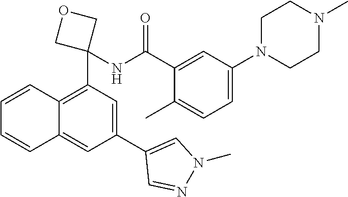 [SMILES: Cc1ccc(N2CCN(C)CC2)cc1C(=O)NC1(c2cc(-c3cnn(C)c3)cc3ccccc23)COC1] | Example 54 using 2- methyl-5-(4- methylpiperazin-1- yl)benzoic acid and 3-(3- (1-methyl-1H-pyrazol-4- yl)naphthalen-1- yl)oxetan-3-amine | ¹H NMR (400 MHz, DMSO-d₆) δ = 9.65 (s, 1H), 8.37 (s, 1H), 8.10-8.00 (m, 2H), 7.97-7.88 (m, 2H), 7.76 (d, J = 8.4 Hz, 1H), 7.55-7.35 (m, 2H), 6.97 (d, J = 8.4 Hz, 1H), 6.85 (dd, J = 2.5, 8.5 Hz, 1H), 6.58 (d, J = 2.4 Hz, 1H), 5.38-5.19 (m, 4H), 3.92 (s, 3H), 3.04-2.93 (m, 4H), 2.44-2.34 (m, 4H), 2.20 (s, 3H), 1.94 (s, 3H). LCMS (ESI): m/z = 496.4, [M + H]⁺. |
| | 2-methyl-N-(3-(3-(1-methyl- | | |
| | 1H-pyrazol-4-yl)naphthalen- | | |
| | 1-yl)oxetan-3-yl)-5-(4- | | |
| | methylpiperazin-1- | | |
| | yl)benzamide | | |
| Ex. 296 | 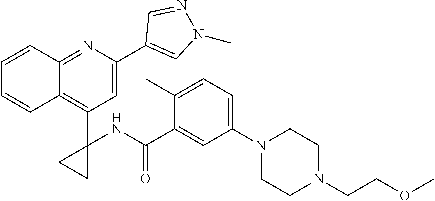 [SMILES: COCCN1CCN(c2ccc(C)c(C(=O)NC3(c4cc(-c5cnn(C)c5)nc5ccccc45)CC3)c2)CC1] | Example 273 using 2- methyl-N-(1-(2-(1- methyl-1H-pyrazol-4- yl)quinolin-4-yl) cyclopropyl)-5- (piperazin-1-yl) benzamide and 1- bromo-2-methoxyethane | ¹H NMR (400 MHz, METHANOL-d₄) δ = 8.56 (d, J = 7.7 Hz, 1H), 8.41 (s, 1H), 8.27 (s, 1H), 8.11-8.06 (m, 2H), 7.76 (t, J = 7.7 Hz, 1H), 7.61 (t, J = 7.6 Hz, 1H), 7.02 (d, J = 8.6 Hz, 1H), 6.90 (dd, J = 2.7, 8.4 Hz, 1H), 6.68 (d, J = 2.7 Hz, 1H), 4.03 (s, 3H), 3.58 (t, J = 5.5 Hz, 2H), 3.36 (s, 3H), 3.14-3.04 (m, 4H), 2.69-2.59 (m, 6H), 2.00 (s, 3H), 1.54-1.40 (m, 4H). LCMS (ESI): m/z = 525.5, [M+ H]⁺. |
| | 5-(4-(2- | | |
| | methoxyethyl)piperazin-1- | | |
| | yl)-2-methyl-N-(1-(2-(1- | | |
| | methyl-1H-pyrazol-4- | | |
| | yl)quinolin-4-yl)cyclopropyl) | | |
| | benzamide | | |
| Ex. 297 | 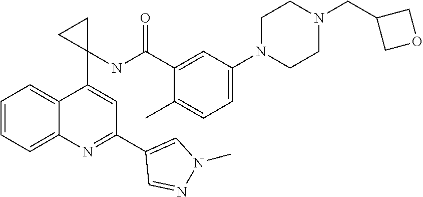 [SMILES: Cc1ccc(N2CCN(CC3COC3)CC2)cc1C(=O)NC1(c2cc(-c3cnn(C)c3)nc3ccccc23)CC1] 2-methyl-N-(1-(2-(1-methyl- 1H-pyrazol-4-yl)quinolin-4- | Example 252 using 2- methyl-N-(1-(2-(1- methyl-1H-pyrazol-4- yl)quinolin-4-yl) cyclopropyl)-5- (piperazin-1-yl) benzamide and oxetane- 3-carbaldehyde instead of formaldehyde | ¹H NMR (400 MHz, MeOD) δ ppm 8.55 (d, J = 7.6 Hz, 1H), 8.41 (s, 1H), 8.27 (s, 1H), 8.09 (s, 1H), 8.07 (d, J = 8.5 Hz, 1H), 7.76 (t, J = 7.6 Hz, 1H), 7.61 (t, J = 7.6 Hz, 1H), 7.02 (d, J = 8.5 Hz, 1H), 6.89 (d, J = 7.9 Hz, 1H), 6.69-6.66 (m, 1H), 4.87- 4.80 (m, 2H), 4.45 (t, J = 6.2 Hz, 2H), 4.03 (s, 3H), 3.10-3.03 (m, 4H), 2.78 (d, J = 7.2 Hz, 2H), 2.58-2.51 (m, 4H), 2.00 (s, 3H), 1.53-1.40 (m, 4H). LCMS (ESI): m/z = 537, [M + H]⁺.. |
| | yl)cyclopropyl)-5-(4- | | |
| | (oxetan-3-ylmethyl) | | |
| | piperazin-1-yl)benzamide | | |
| Ex. 298 | 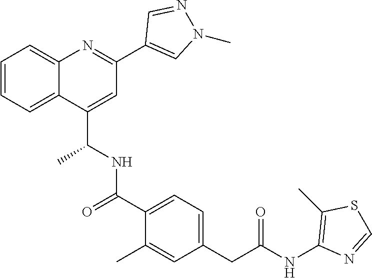 [SMILES: Cc1cc(CC(=O)Nc2ncsc2C)ccc1C(=O)N[C@H](C)c1cc(-c2cnn(C)c2)nc2ccccc12] | Example 236 using (R)- 2-(3-methyl-4-((1-(2-(1- methyl-1H-pyrazol-4- yl)quinolin-4- yl)ethyl)carbamoyl)phen yl)acetic acid and 5- methylthiazol-4-amine | ¹H NMR (400 MHz, DMSO-d₆) δ = 10.17 (s, 1H), 8.95 (br d, J = 7.6 Hz, 1H), 8.78 (s, 1H), 8.42 (s, 1H), 8.23 (d, J = 8.3 Hz, 1H), 8.09 (s, 1H), 7.97 (d, J = 7.9 Hz, 1H), 7.86 (s, 1H), 7.74 (t, J = 7.6 Hz, 1H), 7.58 (t, J = 7.1 Hz, 1H), 7.35 (d, J = 7.6 Hz, 1H), 7.28-7.17 (m, 2H), 5.89 (br t, J = 7.2 Hz, 1H), 3.94 (s, 3H), 3.63 (s, 2H), 3.33-3.30 (m, 2H), 2.29 (s, 3H), 2.18 (s, 3H), 1.61 (d, J = 6.9 Hz, 3H).LCMS (ESI): m/z = 525.2, [M + H]⁺. |
| | (R)-2-methyl-N-(1-(2-(1- | | |
| | methyl-1H-pyrazol-4- | | |
| | yl)quinolin-4-yl)ethyl)-4-(2- | | |
| | ((5-methylthiazol-4- | | |
| | yl)amino)-2- | | |
| | oxoethyl)benzamide | | |
| Ex. 299 | 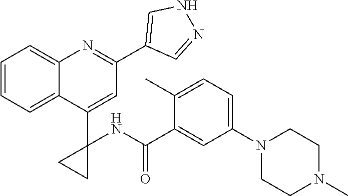 [SMILES: Cc1ccc(N2CCN(C)CC2)cc1C(=O)NC1(c2cc(-c3cn[nH]c3)nc3ccccc23)CC1] | Example 275 using 4- (4,4,5,5-tetramethyl- 1,3,2-dioxaborolan-2-yl)- 1H-pyrazole instead of (3-methyl-1H-pyrazol-5- yl)boronic acid in Step 1 | ¹H NMR (400 MHz, MeOD-d₄) δ = 8.55 (d, J = 7.7 Hz, 1H), 8.52- 8.28 (m, 2H), 8.13 (s, 1H), 8.07 (d, J = 8.4 Hz, 1H), 7.75 (dt, J = 1.3, 7.7 Hz, 1H), 7.62-7.56 (m, 1H), 7.01 (d, J = 8.4 Hz, 1H), 6.88 (dd, J = 2.6, 8.4 Hz, 1H), 6.67 (d, J = 2.6 Hz, 1H), 3.11- 3.04 (m, 4H), 2.59-2.52 (m, 4H), 2.32 (s, 3H), 1.99 (s, 3H), 1.52- 1.40 (m, 4H). LCMS (ESI): m/z = 467.4, [M + H]⁺. |
| | N-(1-(2-(1H-pyrazol-4- | | |
| | yl)quinolin-4- | | |
| | yl)cyclopropyl)-2-methyl-5- | | |
| | (4-methylpiperazin-1- | | |
| | yl)benzamide | | |
| Ex. 300 | 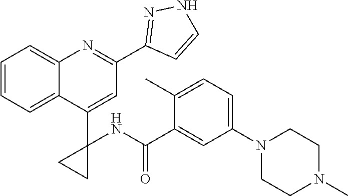 [SMILES: Cc1ccc(N2CCN(C)CC2)cc1C(=O)NC1(c2cc(-c3cc[nH]n3)nc3ccccc23)CC1] | Example 275 using 3- (4,4,5,5-tetramethyl- 1,3,2-dioxaborolan-2-yl)- 1H-pyrazole instead of (3-methyl-1H-pyrazol-5- yl)boronic acid in Step 1 | ¹H NMR (400 MHz, CDCl₃) δ = 8.39 (d, J = 8.13, 1H), 8.20 (s, 1H), 8.14 (d, J = 8.48, 1H), 7.75-7.70(m, 2H), 7.61-7.57 (m, 1H), 7.02-6.98 (m, 2H), 6.80 (dd, J = 8.43, 2.3, 1H), 6.67 (d, J = 2.3, 1H), 6.51 (s, 1H), 3.12- 3.03 (m, 4H), 2.64-2.59 (m, 4H), 2.38 (s, 3H), 2.08 (s, 3H), 1.59- 1.44 (m, 4H). LCMS (ESI): m/z = 467.4, [M + H]⁺. |
| | N-(1-(2-(1H-pyrazol-3- | | |
| | yl)quinolin-4- | | |
| | yl)cyclopropyl)-2-methyl-5- | | |
| | (4-methylpiperazin-1- | | |
| | yl)benzamide | | |
| Ex. 301 | 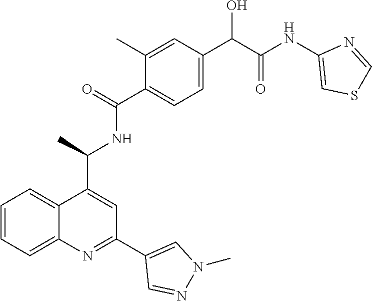 [SMILES: Cc1cc(C(O)C(=O)Nc2cscn2)ccc1C(=O)N[C@H](C)c1cc(-c2cnn(C)c2)nc2ccccc12] | Example 54 using 4-(1- hydroxy-2-oxo-2- (thiazol-4-ylamino)ethyl)- 2-methylbenzoic acid and (R)-1-(2-(1-methyl- 1H-pyrazol-4-yl)quinolin- 4-yl)ethan-1-amine | ¹H NMR (400 MHz, METHANOL-d₄) δ = 8.82 (d, J = 2.3 Hz, 1H), 8.33-8.29 (m, 1H), 8.28-8.23 (m, 1H), 8.15 (d, J = 0.8 Hz, 1H), 8.08-8.03 (m, 1H), 7.83 (s, 1H), 7.78-7.73 (m, 1H), 7.64 (d, J = 2.3 Hz, 1H), 7.63- 7.58 (m, 1H), 7.44 (s, 1H), 7.40- 7.35 (m, 1H), 6.05-5.98 (m, 1H), 5.22 (s, 1H), 4.00-3.98 (m, 3H), 2.35 (s, 3H), 1.71 (d, J = 7.0 Hz, 3H). LCMS (ESI): m/z = 527.3, [M + H]⁺. |
| | 4-(1-hydroxy-2-oxo-2- | | |
| | (thiazol-4-ylamino)ethyl)-2- | | |
| | methyl-N-((R)-1-(2-(1- | | |
| | methyl-1H-pyrazol-4- | | |
| | yl)quinolin-4- | | |
| | yl)ethyl)benzamide | | |
| Ex. 302 | 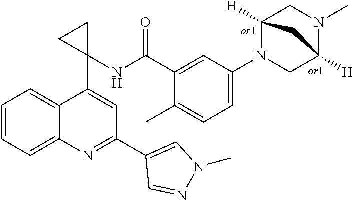 [SMILES: Cc1ccc(N2C[C@@H]3C[C@@H]2CN3C)cc1C(=O)NC1(c2cc(-c3cnn(C)c3)nc3ccccc23)CC1] rel-2-methyl-N-(1-(2-(1- methyl-1H-pyrazol-4- yl)quinolin-4-yl)cyclopropyl)- | Example 252 using rel- 5-((1R,4R)-2,5- diazabicyclo[2.2.1] heptan-2-yl)-2-methyl-N- (1-(2-(1-methyl-1H- pyrazol-4-yl)quinolin-4- yl)cyclopropyl) benzamide, ENT-1 | ¹H NMR (400 MHz, MeOD) δ ppm 8.56 (d, J = 7.7 Hz, 1H), 8.41 (s, 1H), 8.27 (s, 1H), 8.10- 8.05 (m, 2H), 7.76 (t, J = 7.6 Hz, 1H), 7.62 (t, J = 7.5 Hz, 1H), 6.96 (d, J = 8.5 Hz, 1H), 6.55 (d, J = 7.9 Hz, 1H), 6.31 (s, 1H), 4.20 (s, 1H), 4.03 (s, 3H), 3.53 (s, 1H), 3.35 (br d, J = 2.2 Hz, 1H), 3.17 (d, J = 9.8 Hz, 1H), 2.77 (dd, J = 1.7, 10.0 Hz, 1H), 2.69 (d, J = 10.1 Hz, 1H), 2.36 (s, 3H), 1.97 (s, 4H), 1.93- 1.85 (m, 1H), 1.53-1.40 (m, 4H). LCMS (ESI): m/z = 493.4, [M + H]⁺. |
| | 5-((1R,4R)-5-methyl-2,5- | | |
| | diazabicyclo[2.2.1]heptan- | | |
| | 2-yl)benzamide, ENT-1 | | |
| Ex. 303 | 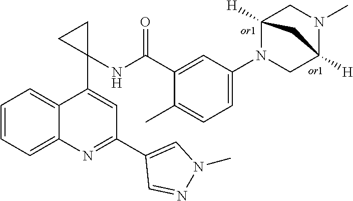 [SMILES: Cc1ccc(N2C[C@@H]3C[C@@H]2CN3C)cc1C(=O)NC1(c2cc(-c3cnn(C)c3)nc3ccccc23)CC1] rel-2-methyl-N-(1-(2-(1- methyl-1H-pyrazol-4- | Example 252 using rel- 5-((1R,4R)-2,5- diazabicyclo[2.2.1] heptan-2-yl)-2- methyl-N-(1-(2- (1-methyl-1H-pyrazol-4- yl)quinolin-4- yl)cyclopropyl) benzamide, ENT-2 | ¹H NMR (400 MHz, MeOD) δ ppm 8.56 (d, J = 8.2 Hz, 1H), 8.41 (s, 1H), 8.27 (s, 1H), 8.11- 8.06 (m, 2H), 7.75 (t, J = 7.6 Hz, 1H), 7.61 (t, J = 7.1 Hz, 1H), 6.95 (d, J = 7.9 Hz, 1H), 6.54 (d, J = 8.6 Hz, 1H), 6.31 (s, 1H), 4.19 (s, 1H), 4.03 (s, 3H), 3.51 (s, 1H), 3.16 (d, J = 9.3 Hz, 1H), 2.76 (dd, J = 1.8, 10.0 Hz, 1H), 2.67 (d, J = 10.0 Hz, 1H), 2.35 (s, 3H), 1.99-1.91 (m, 4H), 1.86 (br d, J = 9.8 Hz, 1H), 1.53-1.40 (m, 4H). LCMS (ESI): m/z = 493.4, [M + H]⁺. |
| | yl)quinolin-4-yl)cyclopropyl) | | |
| | -5-((1R,4R)-5-methyl-2,5- | | |
| | diazabicyclo[2.2.1]heptan- | | |
| | 2-yl)benzamide, ENT-2 | | |
| Ex. 304 | 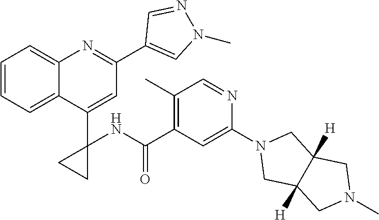 [SMILES: Cc1cnc(N2C[C@H]3CN(C)C[C@H]3C2)cc1C(=O)NC1(c2cc(-c3cnn(C)c3)nc3ccccc23)CC1] | Example 251 using 2- ((3aR,6aS)-5-(tert- butoxycarbonyl) hexahydropyrrolo[3,4- c]pyrrol-2(1H)-yl)-5- methylisonicotinic acid and 1-(2-(1-methyl-1H- pyrazol-4-yl)quinolin-4- yl)cyclopropan-1-amine; then Example 252 | ¹H NMR (400 MHz, MeOD) δ ppm 8.53 (d, J = 7.6 Hz, 1H), 8.41 (s, 1H), 8.27 (s, 1H), 8.12- 8.05 (m, 2H), 7.86-7.73 (m, 2H), 7.61 (t, J = 7.3 Hz, 1H), 6.30 (s, 1H), 4.03 (s, 3H), 3.48-3.40 (m, 2H), 3.32-3.29 (m, 2H), 3.04- 2.94 (m, 2H), 2.83 (br dd, J = 7.1, 9.6 Hz, 2H), 2.42 (dd, J = 3.9, 9.7 Hz, 2H), 2.33 (s, 3H), 1.90 (s, 3H), 1.56-1.41 (m, 4H). LCMS (ESI): m/z = 508.4, [M + H]⁺. |
| | 5-methyl-N-(1-(2-(1-methyl- | | |
| | 1H-pyrazol-4-yl)quinolin-4- | | |
| | yl)cyclopropyl)-2- | | |
| | (3aR,6aS)-5- | | |
| | methylhexahydropyrrolo | | |
| | [3,4-c]pyrrol-2(1H)- | | |
| | yl)isonicotinamide | | |
| Ex. 322 | 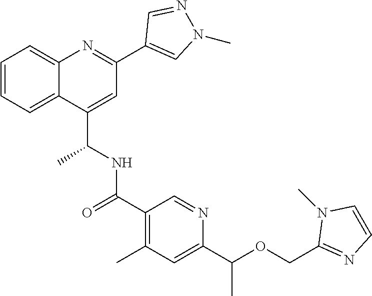 [SMILES: Cc1cc(C(C)OCc2nccn2C)ncc1C(=O)N[C@H](C)c1cc(-c2cnn(C)c2)nc2ccccc12] | Example 321 using 2- (chloromethyl)-1-methyl- 1H-imidazole instead of 4-(chloromethyl)thiazole and tBuOK instead of NaH in THF in step 3 | ¹H NMR (400 MHz, DMSO-d₆) δ = 9.18 (d, J = 7.5 Hz, 1H), 8.52 (s, 1H), 8.44 (s, 1H), 8.23 (d, J = 8.4 Hz, 1H), 8.11 (s, 1H), 7.98 (d, J = 7.9 Hz, 1H), 7.87 (s, 1H), 7.74 (t, J = 7.3 Hz, 1H), 7.60 (t, J = 7.7 Hz, 1H), 7.34 (s, 1H), 7.13 (s, 1H), 6.79 (d, J = 0.9 Hz, 1H), 5.91-5.87 (m, 1H), 4.59- 455 (m, 1H), 4.46 (d, J = 1.3 Hz, 2H), 3.95 (s, 3H), 3.65 (s, 3H), 2.34 (d, J = 1.5 Hz, 3H), 1.64 (d, J = 7.0 Hz, 3H), 1.38 (d, J = 6.4 Hz, 3H). LCMS (ESI): m/z = 510.3, [M + H]⁺. |
| | 4-methyl-6-(1-((1-methyl- | | |
| | 1H-imidazol-2- | | |
| | yl)methoxy)ethyl)-N-((R)-1- | | |
| | (2-(1-methyl-1H-pyrazol-4- | | |
| | yl)quinolin-4- | | |
| | yl)ethyl)nicotinamide | | |
| Ex. 323 | 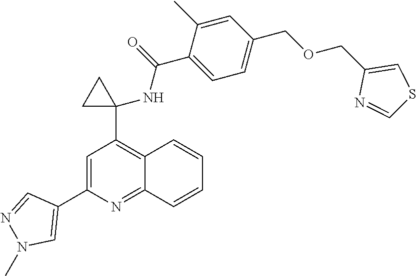 [SMILES: Cc1cc(COCc2cscn2)ccc1C(=O)NC1(c2cc(-c3cnn(C)c3)nc3ccccc23)CC1] | Example 274 using 1-(2- (1-methyl-1H-pyrazol-4- yl)quinolin-4- yl)cyclopropan-1-amine as the amine in Step 6 | ¹H NMR (400 MHz, DMSO-d₆) δ = 9.23 (s, 1H), 9.07 (d, J=1.8 Hz, 1H), 8.60-8.49 (m, 2H), 8.17 (s, 1H), 7.98-7.90 (m, 2H), 7.74-7.66 (m, 1H), 7.66-7.60 (m, 1H), 7.58-7.50 (m, 1H), 7.18-7.11 (m, 2H), 7.11-7.05 (m, 1H), 4.60 (s, 2H), 4.51 (s, 2H), 3.94 (s, 3H), 2.06 (s, 3H), 1.37-135 (m, 4H). LCMS (ESI): m/z = 510.3, [M + H]⁺. |
| | 2-methyl-N-(1-(2-(1-methyl- | | |
| | 1H-pyrazol-4-yl)quinolin-4- | | |
| | yl)cyclopropyl)-4-((thiazol-4- | | |
| | ylmethoxy)methyl) | | |
| | benzamide | | |

### Examples 305-320

The following compounds were synthetized by processes similar to those described herein.

| Ex. # | Structure; Compound Name | Characterization Data |
|---|---|---|
| Ex. 305 | 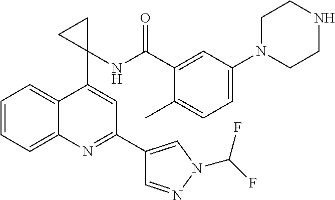 [SMILES: Cc1ccc(N2CCNCC2)cc1C(=O)NC1(c2cc(-c3cnn(C(F)F)c3)nc3ccccc23)CC1] | ¹H NMR (400 MHz, MeOD) δ ppm 8.90 (s, 1H), 8.58 (d, J = 8.1 Hz, 1H), 8.54-8.47 (m, 2H), 8.19 (s, 1H), 8.12 (d, J = 8.4 Hz, 1H), 7.83-7.73 (m, 1H), 7.70-7.45 (m, 2H), 7.09 (d, J = 8.4 Hz, 1H), 6.96 (dd, J = 2.4, 8.4 Hz, 1H), 6.73 (d, J = 2.4 Hz, 1H), 3.31-3.25 (m, 8H), 2.03 (s, 3H), 1.55-1.42 (m, 4H). LCMS (ESI): m/z = 503.4 [M + H]⁺. |
| | N-(1-(2-(1-(difluoromethyl)-1H- | |
| | pyrazol-4-yl)quinolin-4- | |
| | yl)cyclopropyl)-2-methyl-5- | |
| | (piperazin-1-yl)benzamide | |
| Ex. 306 | 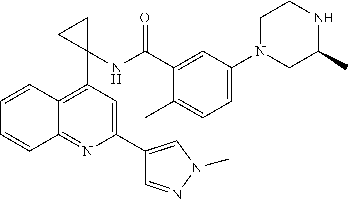 [SMILES: Cc1ccc(N2CCN[C@@H](C)C2)cc1C(=O)NC1(c2cc(-c3cnn(C)c3)nc3ccccc23)CC1] | ¹H NMR (400 MHz, METHANOL-d₄) δ ppm 1.09- 1.17 (m, 3H), 1.39-1.47 (m, 2H), 1.48-1.56 (m, 2H), 1.98-2.05 (m, 3H), 2.20-2.33 (m, 1H), 2.53-2.64 (m, 1H), 2.83-2.96 (m, 2H), 3.00-3.10 (m, 1H), 3.38-3.48 (m, 2H), 4.00-4.05 (m, 3H), 6.60-6.72 (m, 1H), 6.87-6.95 (m, 1H), 7.00-7.06 (m, 1H), 7.56-7.67 (m, 1H), 7.71-7.84 (m, 1H), 8.04-8.13 (m, 2H), 8.22-8.31 (m, 1H), 8.41 (s, 1H), 8.52-8.61 (m, 1H),. LCMS (ESI): m/z = 481.4, [M + H]⁺. |
| | (S)-2-methyl-N-(1-(2-(1-methyl-1H- | |
| | pyrazol-4-yl)quinolin-4- | |
| | yl)cyclopropyl)-5-(3- | |
| | methylpiperazin-1-yl)benzamide | |
| Ex. 307 | 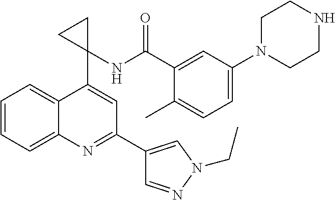 [SMILES: CCn1cc(-c2cc(C3(NC(=O)c4cc(N5CCNCC5)ccc4C)CC3)c3ccccc3n2)cn1] | ¹H NMR (400 MHz, MeOD) δ ppm 8.56 (d, J = 7.8 Hz, 1H), 8.47 (s, 1H), 8.28 (s, 1H), 8.10 (s, 1H), 8.07 (d, J = 8.8 Hz, 1H), 7.76 (t, J = 7.1 Hz, 1H), 7.61 (t, J = 7.1 Hz, 1H), 7.03 (d, J = 8.6 Hz, 1H), 6.90 (d, J = 8.6 Hz, 1H), 6.70-6.66 (m, 1H), 4.32 (q, J = 7.3 Hz, 2H), 3.07-2.99 (m, 4H), 2.99-2.89 (m, 4H), 2.01 (s, 3H), 1.57 (t, J = 7.3 Hz, 3H), 1.53- 1.42 (m, 4H). LCMS (ESI): m/z = 481.4, [M + H]⁺. |
| | N-(1-(2-(1-ethyl-1H-pyrazol-4- | |
| | yl)quinolin-4-yl)cyclopropyl)-2- | |
| | methyl-5-(piperazin-1- | |
| | yl)benzamide | |
| Ex. 308 | 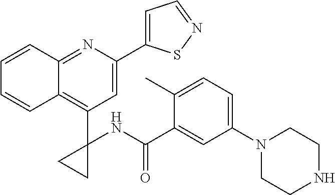 [SMILES: Cc1ccc(N2CCNCC2)cc1C(=O)NC1(c2cc(-c3ccns3)nc3ccccc23)CC1] | ¹H NMR (400 MHz, METHANOL-d4) δ = 8.64-8.57 (m, 2H), 8.29 (s, 1H), 8.14-8.07 (m, 2H), 7.80 (dt, J = 1.3, 7.8 Hz, 1H), 7.72-7.65 (m, 1H), 7.01 (d, J = 8.5 Hz, 1H), 6.89 (dd, J = 2.6, 8.4 Hz, 1H), 6.67 (d, J = 2.8 Hz, 1H), 3.02 (dd, J = 3.5, 6.1 Hz, 4H), 2.95-2.89 (m, 4H), 1.98 (s, 3H), 1.54-1.50 (m, 2H), 1.48-1.43 (m, 2H). LCMS (ESI): m/z = 470.3, [M + H]⁺. |
| | N-(1-(2-(isothiazol-5-yl)quinolin-4- | |
| | yl)cyclopropyl)-2-methyl-5- | |
| | (piperazin-1-yl)benzamide | |
| Ex. 309 | 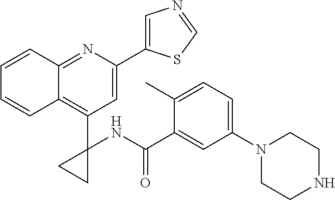 [SMILES: Cc1ccc(N2CCNCC2)cc1C(=O)NC1(c2cc(-c3cncs3)nc3ccccc23)CC1] | ¹H NMR (400 MHz, MeOD-d4) δ = 9.11 (s, 1H), 8.72 (s, 1H), 8.57 (d, J = 7.9 Hz, 1H), 8.30 (s, 1H), 8.08 (d, J = 8.3 Hz, 1H), 7.80-7.74 (m, 1H), 7.67- 7.61 (m, 1H), 7.01 (d, J = 8.4 Hz, 1H), 6.88 (dd, J = 2.6, 8.4 Hz, 1H), 6.67 (d, J = 2.6 Hz, 1H), 3.03- 2.99 (m, 4H), 2.94-2.90 (m, 4H), 1.99 (s, 3H), 1.53- 1.49 (m, 2H), 1.47-1.43 (m, 2H). LCMS (ESI): m/z = 470.4, [M + H]⁺. |
| | 2-methyl-5-(piperazin-1-yl)-N-(1-(2- | |
| | (thiazol-5-yl)quinolin-4- | |
| | yl)cyclopropyl)benzamide | |
| Ex. 310 | 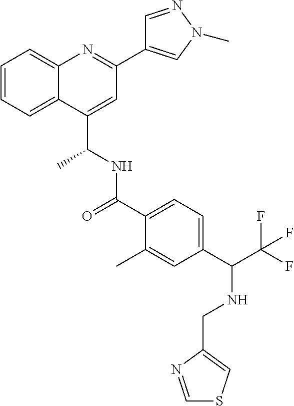 [SMILES: Cc1cc(C(NCc2cscn2)C(F)(F)F)ccc1C(=O)N[C@H](C)c1cc(-c2cnn(C)c2)nc2ccccc12] | ¹H NMR (400 MHz, DMSO-d6) δ = 9.08-8.98 (m, 1H), 9.17-8.96 (m, 1H), 8.42 (s, 1H), 8.23 (d, J = 8.2 Hz, 1H), 8.09 (s, 1H), 7.97 (d, J = 8.3 Hz, 1H), 7.86 (s, 1H), 7.73 (t, J = 7.6 Hz, 1H), 7.57 (t, J = 7.6 Hz, 1H), 7.47 (s, 1H), 7.43-7.33 (m, 3H), 5.89 (br t, J = 7.3 Hz, 1H), 4.46 (br t, J = 8.1 Hz, 1H), 3.94 (s, 3H), 3.87-3.69 (m, 1H), 3.45-3.35 (m, 2H), 2.29 (s, 3H), 1.61 (d, J = 7.0 Hz, 3H). LCMS (ESI): m/z = 565.3, [M + H]⁺. |
| | 2-methyl-N-((R)-1-(2-(1-methyl-1H- | |
| | pyrazol-4-yl)quinolin-4-yl)ethyl)-4- | |
| | (2,2,2-trifluoro-1-((thiazol-4- | |
| | ylmethyl)amino)ethyl)benzamide | |
| Ex. 311 | 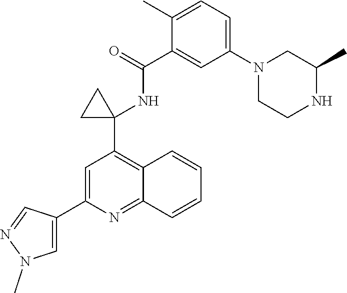 [SMILES: Cc1ccc(N2CCN[C@H](C)C2)cc1C(=O)NC1(c2cc(-c3cnn(C)c3)nc3ccccc23)CC1] | ¹H NMR (400 MHz, METHANOL-d₄) δ ppm 1.06- 1.16 (m, 3H) 1.40-1.47 (m, 2H) 1.49-1.56 (m, 2H) 1.95-2.08 (m, 3H) 2.17-2.33 (m, 1H) 2.53-2.68 (m, 1H) 2.84-2.96 (m, 2H) 2.99-3.12 (m, 1H) 3.39-3.47 (m, 2H) 3.98-4.11 (m, 3H) 6.60-6.71 (m, 1H) 6.86-6.96 (m, 1H) 6.98-7.10 (m, 1H) 7.57-7.67 (m, 1H) 7.73-7.81 (m, 1H) 8.03-8.13 (m, 2H) 8.24-8.31 (m, 1H) 8.36-8.45 (m, 1H) 8.52-8.62 (m, 1H). LCMS (ESI): m/z = 481.4, [M + H]⁺. |
| | (R)-2-methyl-N-(1-(2-(1-methyl-1H- | |
| | pyrazol-4-yl)quinolin-4- | |
| | yl)cyclopropyl)-5-(3- | |
| | methylpiperazin-1-yl)benzamide | |
| Ex. 312 | 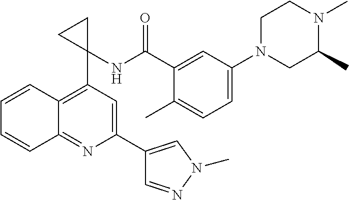 [SMILES: Cc1ccc(N2CCN(C)[C@@H](C)C2)cc1C(=O)NC1(c2cc(-c3cnn(C)c3)nc3ccccc23)CC1] | ¹H NMR (400 MHz, METHANOL-d₄) δ ppm 8.59-8.51 (m, 1H), 8.42 (s, 1H), 8.27 (s, 1H), 8.12-8.04 (m, 2H), 7.81-7.72 (m, 1H), 7.64- 7.56 (m, 1H), 7.03 (d, J = 8.50 Hz, 1H), 6.94-6.86 (m, 1H), 6.73-6.60 (m, 1H), 4.06-3.98 (m, 3H), 3.48-3.36 (m, 2H), 2.96-2.86 (m, 1H), 2.80-2.71 (m, 1H), 2.47-2.36 (m, 2H), 2.36-2.32 (m, 3H), 2.31-2.23 (m, 1H), 2.05-1.93 (m, 3H), 1.55-1.48 (m, 2H), 1.47-1.39 (m, 2H), 1.17-1.09 (m, 3H). LCMS (ESI): m/z = 495.6, [M + H]⁺. |
| | (S)-5-(3,4-dimethylpiperazin-1-yl)- | |
| | 2-methyl-N-(1-(2-(1-methyl-1H- | |
| | pyrazol-4-yl)quinolin-4- | |
| | yl)cyclopropyl)benzamide | |
| Ex. 313 | 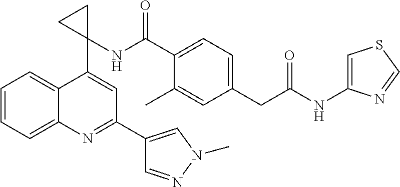 [SMILES: Cc1cc(CC(=O)Nc2cscn2)ccc1C(=O)NC1(c2cc(-c3cnn(C)c3)nc3ccccc23)CC1] | ¹H NMR (400 MHz, METHANOL-d4) δ = 8.66 (d, J = 2.3 Hz, 1H), 8.43 (d, J = 8.4 Hz, 1H), 8.29 (s, 1H), 8.15 (s, 1H), 7.99 (s, 1H), 7.96 (d, J = 8.4 Hz, 1H), 7.71-7.58 (m, 1H), 7.54-7.44 (m, 2H), 7.08- 7.02 (m, 2H), 7.02-6.97 (m, 1H), 3.91 (s, 3H), 3.57 (s, 2H), 2.00 (s, 3H), 1.42-1.29 (m, 4H). LCMS (ESI): m/z = 523.3, [M + H]⁺. |
| | 2-methyl-N-(1-(2-(1-methyl-1H- | |
| | pyrazol-4-yl)quinolin-4- | |
| | yl)cyclopropyl)-4-(2-oxo-2-(thiazol- | |
| | 4-ylamino)ethyl)benzamide | |
| Ex. 314 | 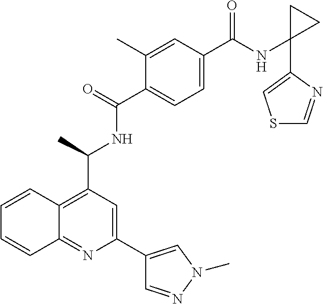 [SMILES: Cc1cc(C(=O)NC2(c3cscn3)CC2)ccc1C(=O)N[C@H](C)c1cc(-c2cnn(C)c2)nc2ccccc12] | ¹H NMR (400 MHz, DMSO-d6) δ = 9.30 (s, 1H), 9.07 (br d, J = 7.8 Hz, 1H), 8.97 (d, J = 2.0 Hz, 1H), 8.44 (s, 1H), 8.24 (br d, J = 8.2 Hz, 1H), 8.11 (s, 1H), 7.98 (br d, J = 8.3 Hz, 1H), 7.88 (s, 1H), 7.83-7.72 (m, 3H), 7.64-7.55 (m, 1H), 7.46 (d, J = 7.8 Hz, 1H), 7.16 (d, J = 2.0 Hz, 1H), 5.91 (br t, J = 7.4 Hz, 1H), 3.95 (s, 3H), 2.34 (s, 3H), 1.64 (br d, J = 6.8 Hz, 3H), 1.52-1.36 (m, 2H), 1.32-1.20 (m, 2H). LCMS (ESI): m/z = 537.3, [M + H]⁺. |
| | (R)-2-methyl-N1-(1-(2-(1-methyl- | |
| | 1H-pyrazol-4-yl)quinolin-4- | |
| | yl)ethyl)-N4-(1-(thiazol-4- | |
| | yl)cyclopropyl)terephthalamide | |
| Ex. 315 | 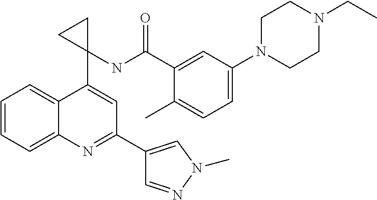 [SMILES: CCN1CCN(c2ccc(C)c(C(=O)NC3(c4cc(-c5cnn(C)c5)nc5ccccc45)CC3)c2)CC1] | ¹H NMR (400 MHz, MeOD) δ ppm 8.56 (d, J = 7.7 Hz, 1H), 8.41 (s, 1H), 8.27 (s, 1H), 8.10 (s, 1H), 8.07 (d, J = 8.6 Hz, 1H), 7.76 (t, J = 7.6 Hz, 1H), 7.61 (t, J = 7.5 Hz, 1H), 7.03 (d, J = 8.5 Hz, 1H), 6.91 (d, J = 8.6 Hz, 1H), 6.69 (s, 1H), 4.03 (s, 3H), 3.17-3.04 (m, 4H), 2.65-2.56 (m, 4H), 2.49 (q, J = 7.2 Hz, 2H), 2.00 (s, 3H), 1.53-1.40 (m, 4H), 1.15 (t, J = 7.3 Hz, 3H). LCMS (ESI): m/z = 495.3, [M + H]⁺. |
| | 5-(4-ethylpiperazin-1-yl)-2-methyl- | |
| | N-(1-(2-(1-methyl-1H-pyrazol-4- | |
| | yl)quinolin-4- | |
| | yl)cyclopropyl)benzamide | |
| Ex. 316 | 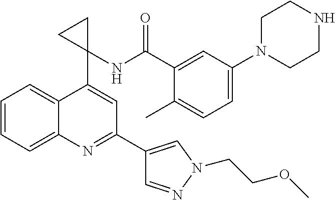 [SMILES: COCCn1cc(-c2cc(C3(NC(=O)c4cc(N5CCNCC5)ccc4C)CC3)c3ccccc3n2)cn1] | ¹H NMR (400 MHz, MeOD) δ ppm 8.56 (d, J = 7.8 Hz, 1H), 8.47 (s, 1H), 8.30 (s, 1H), 8.10 (s, 1H), 8.08 (d, J = 8.7 Hz, 1H), 7.76 (t, J = 7.3 Hz, 1H), 7.61 (t, J = 7.2 Hz, 1H), 7.03 (d, J = 8.6 Hz, 1H), 6.90 (d, J = 8.7 Hz, 1H), 6.69 (s, 1H), 4.43 (t, J = 5.1 Hz, 2H), 3.83 (t, J = 5.1 Hz, 2H), 3.38 (s, 3H), 3.07-2.98 (m, 4H), 2.98-2.89 (m, 4H), 2.01 (s, 3H), 1.54-1.40 (m, 4H). LCMS (ESI): m/z = 511.4, [M + H]⁺. |
| | N-(1-(2-(1-(2-methoxyethyl)-1H- | |
| | pyrazol-4-yl)quinolin-4- | |
| | yl)cyclopropyl)-2-methyl-5- | |
| | (piperazin-1-yl)benzamide | |
| Ex. 317 | 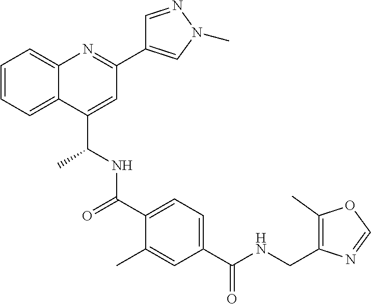 [SMILES: Cc1cc(C(=O)NCc2ncoc2C)ccc1C(=O)N[C@H](C)c1cc(-c2cnn(C)c2)nc2ccccc12] | ¹H NMR (400 MHz, METHANOL-d4) Shift = 8.34 (s, 1H), 8.29 (d, J = 8.6 Hz, 1H), 8.18 (s, 1H), 8.08 (d, J = 8.6 Hz, 1H), 8.03 (s, 1H), 7.86 (s, 1H), 7.81- 7.69 (m, 3H), 7.63 (t, J = 7.3 Hz, 1H), 7.45 (d, J = 7.8 Hz, 1H), 6.05 (q, J = 6.9 Hz, 1H), 4.43 (s, 2H), 4.02 (s, 3H), 2.40 (d, J = 4.9 Hz, 6H), 1.76 (d, J = 7.0 Hz, 3H). LCMS (ESI): m/z = 509.3, [M + H]⁺. |
| | (R)-2-methyl-N1-(1-(2-(1-methyl- | |
| | 1H-pyrazol-4-yl)quinolin-4- | |
| | yl)ethyl)-N4-((5-methyloxazol-4- | |
| | yl)methyl)terephthalamide | |
| Ex. 318 | 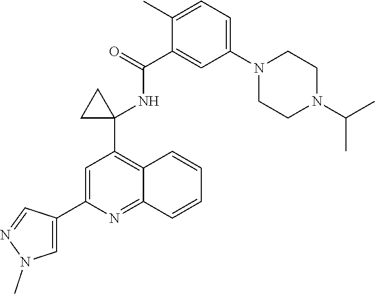 [SMILES: Cc1ccc(N2CCN(C(C)C)CC2)cc1C(=O)NC1(c2cc(-c3cnn(C)c3)nc3ccccc23)CC1] | ¹H NMR (400 MHz, METHANOL-d4) δ = 8.56 (d, J = 7.7 Hz, 1H), 8.41 (s, 1H), 8.27 (s, 1H), 8.09 (s, 1H), 8.07 (d, J = 8.2 Hz, 1H), 7.76 (t, J = 7.7 Hz, 1H), 7.61 (t, J = 7.3 Hz, 1H), 7.03 (d, J = 8.4 Hz, 1H), 6.90 (dd, J = 2.6, 8.4 Hz, 1H), 6.69 (d, J = 2.6 Hz, 1H), 4.03 (s, 3H), 3.13-3.06 (m, 4H), 2.75-2.64 (m, 5H), 2.00 (s, 3H), 1.53-1.41 (m, 4H), 1.12 (d, J = 6.6 Hz, 6H). LCMS (ESI): m/z = 509.4, [M + H]⁺. |
| | 5-(4-isopropylpiperazin-1-yl)-2- | |
| | methyl-N-(1-(2-(1-methyl-1H- | |
| | pyrazol-4-yl)quinolin-4- | |
| | yl)cyclopropyl)benzamide | |
| Ex. 319 | 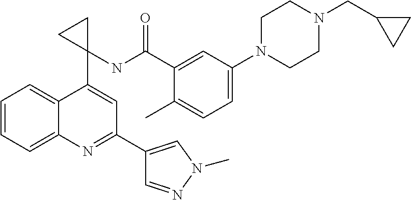 [SMILES: Cc1ccc(N2CCN(CC3CC3)CC2)cc1C(=O)NC1(c2cc(-c3cnn(C)c3)nc3ccccc23)CC1] | ¹H NMR (400 MHz, MeOD) δ ppm 8.56 (d, J = 8.1 Hz, 1H), 8.41 (s, 1H), 8.27 (s, 1H), 8.10 (s, 1H), 8.07 (d, J = 8.6 Hz, 1H), 7.76 (t, J = 7.7 Hz, 1H), 7.61 (t, J = 7.6 Hz, 1H), 7.03 (d, J = 8.5 Hz, 1H), 6.91 (d, J = 8.0 Hz, 1H), 6.69 (s, 1H), 4.03 (s, 3H), 3.17-3.08 (m, 4H), 2.76-2.63 (m, 4H), 2.33 (d, J = 6.6 Hz, 2H), 2.01 (s, 3H), 1.54-1.40 (m, 4H), 0.97- 0.88 (m, 1H), 0.62-0.54 (m, 2H), 0.19 (q, J = 4.9 Hz, 2H). LCMS (ESI): m/z = 521, [M + H]⁺. |
| | 5-(4-(cyclopropylmethyl)piperazin- | |
| | 1-yl)-2-methyl-N-(1-(2-(1-methyl- | |
| | 1H-pyrazol-4-yl)quinolin-4- | |
| | yl)cyclopropyl)benzamide | |
| Ex. 320 | 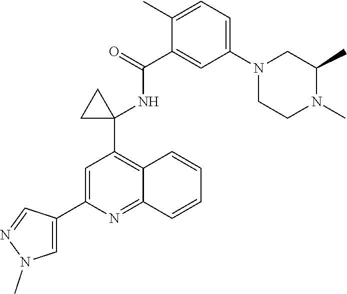 [SMILES: Cc1ccc(N2CCN(C)[C@H](C)C2)cc1C(=O)NC1(c2cc(-c3cnn(C)c3)nc3ccccc23)CC1] | ¹H NMR (400 MHz, METHANOL-d4) δ ppm 8.60- 8.52 (m, 1H), 8.46-8.37 (m, 1H), 8.29-8.20 (m, 1H), 8.12-8.02 (m, 2H), 7.82-7.72 (m, 1H), 7.67-7.56 (m, 1H), 7.09-6.96 (m, 1H), 6.94-6.86 (m, 1H), 6.72-6.64 (m, 1H), 4.10-3.99 (m, 3H), 3.48-3.36 (m, 2H), 2.96-2.87 (m, 1H), 2.82-2.70 (m, 1H), 2.47-2.37 (m, 2H), 2.36-2.31 (m, 3H), 2.31-2.23 (m, 1H), 2.04-1.97 (m, 3H), 1.55-1.48 (m, 2H), 1.47-1.39 (m, 2H), 1.22-1.05 (m, 3H). LCMS (ESI): m/z = 495.5, [M + H]⁺. |
| | (R)-5-(3,4-dimethylpiperazin-1-yl)- | |
| | 2-methyl-N-(1-(2-(1-methyl-1H- | |
| | pyrazol-4-yl)quinolin-4- | |
| | yl)cyclopropyl)benzamide | |

### Example 324: rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-1

### Example 325: rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-2

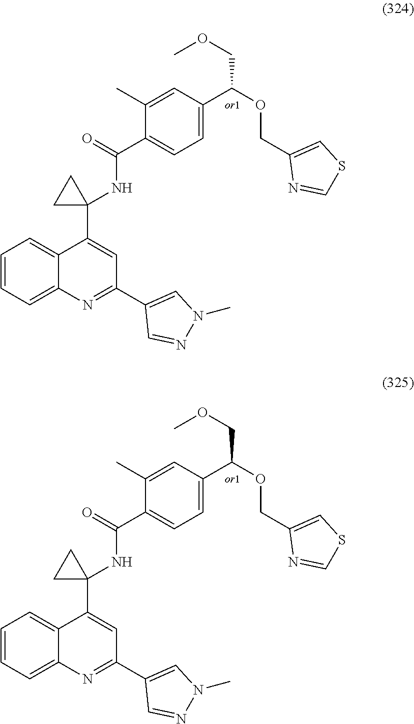 [SMILES: COC[C@H](OCc1cscn1)c1ccc(C(=O)NC2(c3cc(-c4cnn(C)c4)nc4ccccc34)CC2)c(C)c1 ; COC[C@@H](OCc1cscn1)c1ccc(C(=O)NC2(c3cc(-c4cnn(C)c4)nc4ccccc34)CC2)c(C)c1]

### Step 1: Preparation of methyl 4-(1,2-dihydroxyethyl)-2-methylbenzoate

To a yellow solution of methyl 2-methyl-4-vinylbenzoate (9700 mg, 55.05 mmol) in H₂O (24.0 mL) and THF (80 mL) was added N-methyl-morpholine-N-oxide (9670 mg, 82.6 mmol), followed by OsO₄ (1000 mg, 3.933 mmol) in THF (40 mL) at 20° C. Then the black mixture was stirred at 20° C. for 3 h. sThe reaction mixture was then poured into an aqueous solution of sodium thiosulfate (80 mL) and then extracted with EtOAc (300 mL). The combined organic layer was then washed with aqueous solution of sodium thiosulfate (80 mL), dried over anhydrous Na₂SO₄ and evaporated after passing potassium iodide-starch paper test. The crude was then purified by chromatography (silica gel column 80 g, EAPE=0-50%, 254 nm UV) to give methyl 4-(1,2-dihydroxyethyl)-2-methylbenzoate (9.2 g, 79.5%) as a colorless oil. ¹H NMR (400 MHz, CHLOROFORM-d) δ=7.92 (d, J=7.8 Hz, 1H), 7.31-7.22 (m, 2H), 4.85 (dd, J=3.4, 7.9 Hz, 1H), 3.95-3.78 (m, 4H), 3.72-3.61 (m, 1H), 2.77 (br d, J=7.5 Hz, 1H), 2.62 (s, 3H), 2.19 (br s, 1H).

### Step 2: Preparation of methyl 4-(2-((tert-butyldimethylsilyl)oxy)-1-hydroxyethyl)-2-methylbenzoate

To a yellow solution of methyl 4-(1,2-dihydroxyethyl)-2-methylbenzoate (7200 mg, 34.25 mmol) in DMF (171 mL) was added 1H-imidazole (3260 mg, 47.9 mmol). tert-Butyldimethylsilyl chloride (5680 mg, 37.7 mmol) was added to the reaction at 0° C. The reaction was then stirred at 0° C. for 20 minutes before being stirred at 20° C. for 3 h. The reaction mixture was partitioned between EtOAc and H₂O (400 mL/200 mL). The ethyl acetate layer was separated and washed with brine (100 mL), dried over anhydrous Na₂SO₄, evaporated, then purified by a Biotage (220 g, EtOAc/PE=0˜2/1) to give methyl 4-(2-((tert-butyldimethylsilyl)oxy)-1-hydroxyethyl)-2-methylbenzoate (9.2 g, 82.8%) as a colorless oil. ¹H NMR of compound 2:1H NMR (400 MHz, CHLOROFORM-d) δ=7.92 (d, J=7.9 Hz, 1H), 7.32-7.23 (m, 2H), 4.77 (dd, J=3.5, 8.3 Hz, 1H), 3.91 (s, 3H), 3.80 (dd, J=3.6, 10.1 Hz, 1H), 3.55 (dd, J=8.4, 10.1 Hz, 1H), 3.00 (br s, 1H), 2.63 (s, 3H), 096-0.90 (m, 9H), 0.13-0.04 (m, 6H).

### Step 3: Preparation of methyl 4-(2-((tert-butyldimethylsilyl)oxy)-1-(thiazol-4-ylmethoxy ethyl)-2-methylbenzoate

To a solution of methyl 4-(2-((tert-butyldimethylsilyl)oxy)-1-hydroxyethyl)-2-methylbenzoate (3160 mg, 9.73 mmol) in DMF (48.7 mL) were added 4-(chloromethyl)thiazole (1300.0 mg, 9.731 mmol) and NaH (584 mg, 14.6 mmol). The mixture was then stirred at 20° C. for 3 h at which time, the LCMS showed a main peak with desired mass. The reaction was then quenched with 20 mL aq. NH₄Cl and extracted with EtOAc (30 mL×2). The combined organic layer was washed with brine (40 mL), dried over Na₂SO₄ and concentrated to give a crude, which was then purified by using a Biotage (silica gel column 40 g, 1% NH₃—H₂O in MeOH/EtOAc=0˜30%, 254 nm UV) to give the methyl 4-(2-((tert-butyldimethylsilyl)oxy)-1-(thiazol-4-ylmethoxy)ethyl)-2-methylbenzoate (1.6 g, y39.0%) as a colorless oil. LCMS (ESI): m/z=422.2 [M+H]⁺.

### Step 4: Preparation of methyl 4-(2-hydroxy-1-(thiazol-4-ylmethoxy)ethyl)-2-methylbenzoate

To a mix of methyl 4-(2-((tert-butyldimethylsilyl)oxy)-1-(thiazol-4-ylmethoxy)ethyl)-2-methylbenzoate (150 mg, 0.356 mmol) in DCM (3 mL) was added TFA (1.5 mL). The reaction was stirred at 20° C. for 4 h, then evaporated and partitioned between aq. NaHCO₃ and EtOAc (20 mL/20 mL). The organic layer was separated and the aqueous layer was extracted with EtOAc (20 mL) again. The combined organic layer was dried over Na₂SO₄, filtered, evaporated, and purified by chromatography (silica gel column 4 g, MeOH/EtOAc=0˜30% 254 nm UV) to give methyl 4-(2-hydroxy-1-(thiazol-4-ylmethoxy)ethyl)-2-methylbenzoate (90 mg, 82%) as a colorless oil. LCMS (ESI): m/z=308.2 [M+H]⁺.

### Step 5: Preparation of methyl 4-(2-methoxy-1-(thiazol-4-ylmethoxy)ethyl)-2-methylbenzoate

To a solution of methyl 4-(2-hydroxy-1-(thiazol-4-ylmethoxy)ethyl)-2-methylbenzoate (60 mg, 0.20 mmol) in DMF (2.00 mL) were added CH₃I (36.0 mg, 0.254 mmol) and NaH (11.7 mg, 0.293 mmol) at 0° C. The mixture was then stirred at 20° C. for 2 h. The mixture was combined with another batch (from 30 mg of methyl 4-(2-hydroxy-1-(thiazol-4-ylmethoxy)ethyl)-2-methylbenzoate) for workup, added aq. sat. NH₄Cl (10 mL) at 0° C., then extracted with EtOAc (10 mL×3). The combined organic layer was dried over Na₂SO₄ and evaporated to give methyl 4-(2-methoxy-1-(thiazol-4-ylmethoxy)ethyl)-2-methylbenzoate (30 mg, 32.0%) as a brown gum. LCMS (ESI): m/z=322.1 [M+H]⁺.

### Step 6: Preparation of 4-(2-methoxy-1-(thiazol-4-ylmethoxy)ethyl)-2-methylbenzoic acid

To a stirred solution of methyl 4-(2-methoxy-1-(thiazol-4-ylmethoxy)ethyl)-2-methylbenzoate (30.0 mg, 0.093 mmol) in THF (1.0 mL), H₂O (0.5 mL) and MeOH (1.0 mL) was added LiOH·H₂O (11.8 mg, 0.280 mmol) at 20° C. The reaction was stirred at 50° C. for 2 h then adjusted to pH=3˜5 by 1 N HCl and extracted with EtOAc (5 mL×3). The combined organic layer was dried over Na₂SO₄ and evaporated to give 4-(2-methoxy-1-(thiazol-4-ylmethoxy)ethyl)-2-methylbenzoic acid (30 mg, 100%) as a yellow gum. LCMS (ESI): m/z=308.2 [M+H]⁺.

### Step 7: Preparation of rac-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide

To a solution of 4-(2-methoxy-1-(thiazol-4-ylmethoxy)ethyl)-2-methylbenzoic acid (98.8 mg, 0.322 mmol) in DMF (4 mL) were added TEA (97.6 mg, 0.965 mmol) and HATU (171 mg, 0.450 mmol). The mixture was stirred for 10 minutes before 1-(2-(1-methyl-1H-pyrazol-4-yl)quinolin-4-yl)cyclopropan-1-amine (85 mg, 0.32 mmol, solid) was added to it at 20° C.). The reaction mixture was stirred at 20° C. for 1 h then purified via the prep-HPLC to give rac-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide (80.0 mg, 45%) as a white solid. LCMS (ESI): m/z=554.3 [M+H]⁺.

### Prep-HPLC Condition:

Column: ACSSH-CE C18 150×40 mm;
Mobile phase: water (ammonia hydroxide v/v)-MeCN; B %: 28%-68%,
Flow Rate (mL/min): 60 mL/min.

### Step 8: Preparation of rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-1 and ENT-2

The racemic rac-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide (80 mg, 0.14 mmol) was separated via chiral SFC to afford rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-2 (27 mg, 34%, peak 1) and rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-1 (25 mg, 31%, peak 2) as white solids.

### Chiral SFC Condition:

Column: DAICEL CHIRALPAK AD (250 mm*30 mm, 10 μm);
Mobile phase: CO₂-EtOH (0.1% NH₃·H₂O); B %: 40%-40%,
Flow Rate (mL/min): 80 mL/min.

rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-2. ¹H NMR (400 MHz, DMSO-d₆) δ9.25 (s, 1H), 9.04 (d, J=1.96 Hz, 1H), 856 (d, J=8.01 Hz, 1H), 8.53 (s, 1H), 8.17 (s, 1H), 7.92-7.97 (m, 2H), 7.70 (t, J=7.67 Hz, 1H), 7.52-7.59 (m, 2H), 7.15 (s, 1H), 7.17 (d, J=9.10 Hz, 1H), 7.06-7.11 (m, 1H), 4.61 (dd, J=4.22, 6.91 Hz, 1H), 4.46 (s, 2H), 3.94 (s, 3H), 3.50 (dd, J=7.09, 10.51 Hz, 1H), 3.34-3.40 (m, 1H), 3.22 (s, 3H), 2.03-2.07 (m, 3H), 1.35 (br d, J=5.38 Hz, 4H). LCMS (ESI): m/z=554.3 [M+H]⁺.

rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-1. ¹H NMR (400 MHz, DMSO-d6) δ9.25 (s, 1H), 9.04 (d, J=1.96 Hz, 1H), 8.56 (d, J=8.01 Hz, 1H), 8.53 (s, 1H), 8.18 (s, 1H), 7.92-7.97 (m, 2H), 7.70 (t, J=7.27 Hz, 1H), 7.52-7.59 (m, 2H), 7.15 (s, 1H), 7.17 (d, J=8.91 Hz, 1H), 7.08 (d, J=7.58 Hz, 1H), 4.61 (dd, J=4.34, 7.03 Hz, 1H), 4.46 (s, 2H), 3.94 (s, 3H), 3.51 (dd, J=7.15, 10.45 Hz, 1H), 3.35-3.39 (m, 1H), 3.22 (s, 3H), 2.05 (s, 3H), 1.30-1.41 (m, 4H). LCMS (ESI): m/z=554.4 [M+H]⁺.

### Example 326: 4-(2-methoxy-3-(pyridin-2-yl)propyl)-2-methyl-N-(1-(2-(1-methyl-1H-pyrazol-4-yl)quinolin-4-yl)cyclopropyl)benzamide

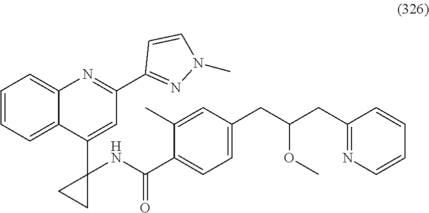 [SMILES: COC(Cc1ccc(C(=O)NC2(c3cc(-c4ccn(C)n4)nc4ccccc34)CC2)c(C)c1)Cc1ccccn1]

### Step 1: Preparation of 2-(4-bromo-3-methylphenyl)-N-methoxy-N-methylacetamide

To a solution of 2-(4-bromo-3-methylphenyl)acetic acid (1000.0 mg, 4.365 mmol) and N,O-dimethylhydroxylamine (639 mg, 6.55 mmol) in DMF (21.8 mL) were added DIEA (3390 mg, 26.2 mmol) and HATU (2490 mg, 6.55 mmol) at 25° C. The reaction was stirred at 25° C. for 12 h then quenched with water (20 mL), and extracted with ethyl acetate (20 mL×3). The combined organic layer was dried over Na₂SO₄, filtered, evaporated to give a crude, which was purified by chromatography (20 g silica gel column, EtOAc/PE from 0 to 50%) to give 2-(4-bromo-3-methylphenyl)-N-methoxy-N-methylacetamide (1.1 g, 92.6%) as an oil. LCMS (ESI): m/z=274.0 [M+H]⁺.

### Step 2: Preparation of 1-(4-bromo-3-methylphenyl)-3-(pyridin-2-yl)propan-2-one

To a solution of 2-methylpyridine (548 mg, 5.88 mmol) in THF (10 mL) cooled to −78° C. was added sec-BuLi (433 mg, 6.76 mmol) at −78° C. The reaction was stirred for 15 min and then 2-(4-bromo-3-methylphenyl)-N-methoxy-N-methylacetamide (800 mg, 2.94 mmol) in THF (4.7 mL) was added dropwise to it. After that, the reaction was quenched by aq. NH₄Cl (20 mL), extracted with EtOAc (20 mL×3). The combined organic layer was dried over Na₂SO₄ then evaporated to give a crude, which was purified by chromatography (20 g silica gel column, EtOAc/PE from 0 to 50%) to give 1-(4-bromo-3-methylphenyl)-3-(pyridin-2-yl)propan-2-one (300 mg, 33.5%) as an oil. ¹H NMR (400 MHz, CHLOROFORM-d) δ=8.59 (d, J=4.2 Hz, 1H), 8.17 (d, J=5.5 Hz, 1H), 7.66 (dt, J=1.9, 7.6 Hz, 1H), 7.60-7.52 (m, 1H), 7.51-7.44 (m, 1H), 7.05 (s, 1H), 7.03 (br d, J=2.0 Hz, 1H), 6.95-6.85 (m, 1H), 3.97 (s, 2H), 3.78 (s, 2H), 3.53 (s, 1H), 2.43-2.34 (m, 3H). LCMS (ESI): m/z=306.0 [M+H]⁺.

### Step 3: Preparation of 1-(4-bromo-3-methylphenyl)-3-(pyridin-2-yl)propan-2-ol

To a solution of 1-(4-bromo-3-methylphenyl)-3-(pyridin-2-yl)propan-2-one (580 mg, 1.91 mmol) in MeOH (9.53 mL) was added NaBH₄ (108 mg, 2.86 mmol) portion wise at 20° C. After the completion of the addition, the mixture was stirred at 20° C. for 2 h then quenched with H₂O (10 mL) and extracted with EtOAc (30 mL×3), The organic layer was evaporated in vacuo to give a crude, which was purified by chromatogrphy (EA/PE from 0% to 50%) to give 1-(4-bromo-3-methylphenyl)-3-(pyridin-2-yl)propan-2-ol (570 mg, 97.6%) as an oil. ¹H NMR (400 MHz, CHLOROFORM-d) δ=8.50 (d, J=4.2 Hz, 1H), 7.64 (dt, J=1.8, 7.7 Hz, 1H), 7.46 (d, J=8.1 Hz, 1H), 7.21-7.10 (m, 3H), 6.96 (dd, J=2.1, 8.1 Hz, 1H), 4.30 (dtd, J=2.9, 6.4, 9.0 Hz, 1H), 2.96-2.72 (m, 4H), 2.40 (s, 3H). LCMS (ESI): m/z=306.0 [M+H]⁺.

### Step 4: Preparation of ethyl 4-(2-hydroxy-3-(pyridin-2-yl)propyl)-2-methylbenzoate

To a solution of 1-(4-bromo-3-methylphenyl)-3-(pyridin-2-yl)propan-2-ol (480 mg, 1.57 mmol) in EtOH (10.0 mL) were added TEA (476 mg, 4.70 mmol) and PdCl₂(dppf) (172 mg, 0.235 mmol) at 50° C. The mixture was degassed with nitrogen then stirred with CO 50 Psi at 80° C. for 48 h. After that, the reaction was filtered. The filtrate was evaporated to give a crude, which was purified by a flash column (20 g silica gel, eluted with PE/EA from 0% to 50%) to give ethyl 4-(2-hydroxy-3-(pyridin-2-yl)propyl)-2-methylbenzoate (310 mg, 66.1%) as an oil. ¹H NMR (400 MHz, CHLOROFORM-d) δ=8.51 (d, J=4.2 Hz, 1H), 7.89 (d, J=8.4 Hz, 1H), 7.64 (dt, J=1.8, 7.7 Hz, 2H), 7.54 (s, 1H), 7.22-7.08 (m, 6H), 5.41 (br s, 1H), 4.41-4.31 (m, 5H), 3.01-2.77 (m, 6H), 2.61 (s, 5H), 1.40 (t, J=7.1 Hz, 5H). LCMS (ESI): m/z=300.0 [M+H]⁺.

### Step-5: Preparation of ethyl 4-methoxy-3-(pyridin-2-yl)propyl)-2-methylbenzoate

To a solution of ethyl 4-(2-hydroxy-3-(pyridin-2-yl)propyl)-2-methylbenzoate (80 mg, 0.27 mmol) in THF (2.0 mL) were added NaH (19.2 mg, 0.802 mmol) and CH₃I (75.9 mg, 0.534 mmol). The suspension was stirred at 20° C. for 4 h then quenched with aq. NH₄Cl (10 mL) and extracted with EtOAc (10 mL×3). The combined organic layer was dried over Na₂SO₄, filtered, and evaporated to give a crude, which was purified via column chromatography to give ethyl 4-(2-methoxy-3-(pyridin-2-yl)propyl)-2-methylbenzoate (52%) as an oil. LCMS (ESI): m/z=314.1 [M+H]⁺.

### Step 6: Preparation of 4-(2-methoxy-3-(pyridin-2-yl)propyl)-2-methylbenzoic acid

To a solution of ethyl 4-(2-methoxy-3-(pyridin-2-yl)propyl)-2-methylbenzoate (60 mg, 0.19 mmol) in EtOH (2 mL) and H₂O (1 mL) was added NaOH (19.1 mg, 0.479 mmol). The reaction was stirred at 50° C. for 2 h then evaporated and acidified to pH=−5 with 1N HCl. After that, the mixture was extracted with EtOAc (10 mL×3). The combined organic layer was dried over Na₂SO₄, filtered, and evaporated to give a crude, which was purified by column chromatography (4 g silica gel, eluted with PE/EtOAc from 10% to 100%) to give 4-(2-methoxy-3-(pyridin-2-yl)propyl)-2-methylbenzoic acid (45 mg, 82%) as an oil. LCMS (ESI): m/z=286.1 [M+H]⁺.

### Step 7: Preparation of 4-(2-methoxy-3-(pyridin-2-yl)propyl)-2-methyl-N-(1-(2-(1-methyl-1H-pyrazol-4-yl)quinolin-4-yl)cyclopropyl)benzamide

To a solution of 4-(2-methoxy-3-(pyridin-2-yl)propyl)-2-methylbenzoic acid (45.0 mg, 0.16 mmol) in DMF (2.0 mL) were added DIEA (122 mg, 0.946 mmol) and HATU (89.9 mg, 0.237 mmol) at 25° C. The reaction mixture was stirred at 25° C. for 10 min before 1-(2-(1-methyl-1H-pyrazol-4-yl)quinolin-4-yl)cyclopropan-1-amine (41.7 mg, 0.158 mmol) was added to it. The reaction mixture was then purified via the prep-HPLC to afford 4-(2-methoxy-3-(pyridin-2-yl)propyl)-2-methyl-N-(1-(2-(1-methy-1H-pyrazol-4-yl)quinolin-4-yl)cyclopropyl)benzamide (20 mg, 24%) as a white solid after lyophilization. ¹H NMR (400 MHz, DMSO-d6) δ=9.20 (s, 1H), 8.58 (d, J=8.0 Hz, 1H), 8.54 (s, 1H), 8.46 (d, J=4.8 Hz, 1H), 8.18 (s, 1H), 7.98-7.91 (m, 2H), 7.74-7.64 (m, 2H), 7.59-7.52 (m, 1H), 7.23 (d, J=7.9 Hz, 1H), 7.19 (dd, J=53, 6.9 Hz, 1H), 7.05-6.97 (m, 3H), 3.94 (s, 3H), 3.89-3.77 (m, 1H), 3.42-3.37 (m, 1H), 3.13 (s, 3H), 2.87-2.64 (m, 5H), 2.06 (s, 3H), 147-1.24 (m, 4H). LCMS (ESI): m/z=532.3 [M+H].

### Prep-HPLC Condition:

Column: C18 150*30*5 um
Mobile phase: water (ammonium hydroxide v/v)-MeCN; B %: 38%-58%,
Flow Rate (mL/min) 35 mL/min.

### Example 327: 4-(2-hydroxy-3-(pyridin-2-yl)propyl)-2-methyl-N-(1-(2-(1-methyl-1H-pyrazol-4-yl)quinolin-4-yl)cyclopropyl)benzamide

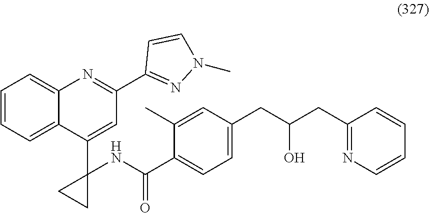 [SMILES: Cc1cc(CC(O)Cc2ccccn2)ccc1C(=O)NC1(c2cc(-c3ccn(C)n3)nc3ccccc23)CC1]

### Step 1: Preparation of 4-(2-hydroxy-3-(pyridin-2-yl)propyl)-2-methylbenzoic acid

To a solution of ethyl 4-(2-hydroxy-3-(pyridin-2-yl)propyl)-2-methylbenzoate (80 mg, 0.27 mmol) in EtOH (1 mL) and H2O (1 mL) was added NaOH (26.7 mg, 0.668 mmol). The reaction was stirred at 50° C. for 2 h then evaporated. After that, the mixture was acidified to pH=˜5 with 1N HCl and extracted with EtOAc (10 mL×3). The combined organic layer was dried over Na₂SO₄, filtered, and evaporated to give a crude, which was purified by column chromatography (4 g silica gel, eluted with PE/EtOAc from 10% to 100%) to give 4-(2-hydroxy-3-(pyridin-2-yl)propyl)-2-methylbenzoic acid (70 mg, 97%) as an oil. LCMS (ESI): m/z=272.1 [M+H]⁺.

### Step 2: Preparation of 4-(2-hydroxy-3-(pyridin-2-yl)propyl)-2-methyl-N-(1-(2-(1-methyl-1H-pyrazol-4-yl)quinolin-4-yl)cyclopropyl)benzamide

To a solution of 4-(2-hydroxy-3-(pyridin-2-yl)propyl)-2-methylbenzoic acid (50.0 mg, 0.19 mmol) in DMF (2.0 mL) were added DIEA (151 mg, 1.17 mmol) and HATU (111 mg, 0.292 mmol) at 25° C. The reaction mixture was stirred at 25° C. for 10 min before 1-(2-(1-methyl-1H-pyrazol-4-yl)quinolin-4-yl)cyclopropan-1-amine (51.4 mg, 0.194 mmol) was added to it. The reaction mixture was then purified via the prep-HPLC to afford 4-(2-hydroxy-3-(pyridin-2-yl)propyl)-2-methyl-N-(1-(2-(1-methyl-1H-pyrazol-4-yl)quinolin-4-yl)cyclopropyl)benzamide (27.87 mg, 28%) as a white solid after lyophilization. ¹H NMR (400 MHz, DMSO-d₆) δ=9.19 (s, 1H), 8.58 (d, J=7.9 Hz, 1H), 8.54 (s, 1H), 8.45 (d, J=4.0 Hz, 1H), 8.18 (s, 1H), 7.98-7.92 (m, 2H), 7.74-7.64 (m, 2H), 7.55 (t, J=7.6 Hz, 1H), 7.23 (d, J=7.9 Hz, 1H), 7.18 (dd, J=5.4, 7.0 Hz, 1H), 7.04-6.97 (m, 3H), 4.78 (d, J=5.6 Hz, 1H), 4.10-3.99 (m, 1H), 3.95 (s, 3H), 3.50-3.37 (m, 1H), 2.81-2.61 (m, 5H), 2.05 (s, 3H), 1.35 (br d, J=7.1 Hz, 4H). LCMS (ESI): m/z=518.4 [M+H]⁺.

### Prep-HPLC Condition:

Column: C18 150*40 mm
Mobile phase: water (ammonium hydroxide v/v)-MeCN; B %: 17%-57%,
Flow Rate (mL/min): 60 mL/min.

### Example 328: 2-methyl-N-(1-(2-(1-(2-(methylsulfonyl)ethyl)-1H-pyrazol-4-yl)quinolin-4-yl)cyclopropyl)-4-((thiazol-4-ylmethoxy)methyl)benzamide

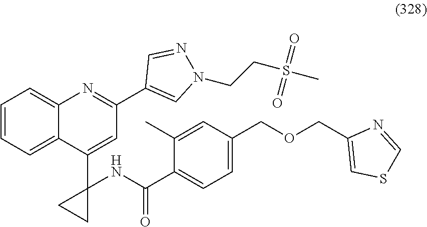 [SMILES: Cc1cc(COCc2cscn2)ccc1C(=O)NC1(c2cc(-c3cnn(CCS(C)(=O)=O)c3)nc3ccccc23)CC1]

### Step 1: Preparation of N-(1-(2-chloroquinolin-4-yl)cyclopropyl)-2-methyl-4-((thiazol-4-ylmethoxy)methyl)benzamide

To a solution of 2-methyl-4-((thiazol-4-ylmethoxy)methyl)benzoic acid (1360 mg, 4.39 mmol) in pyridine (40.0 mL) were added EDCI (1260 mg, 6.58 mmol) and 1-(2-chloroquinolin-4-yl)cyclopropan-1-amine (960.0 mg, 4.39 mmol) at 25° C. The reaction was stirred at 25° C. for 18 h. After that, the reaction was evaporated to give a crude, which was purified by column chromatography (20 g silica gel column, EA/PE from 0% to 70%) to give N-(1-(2-chloroquinolin-4-yl)cyclopropyl)-2-methyl-4-((thiazol-4-ylmethoxy)methyl)benzamide (1600 mg, 78.6%) as a yellow solid. ¹H NMR (400 MHz, METHANOL-d4) δ=9.00 (d, J=2.0 Hz, 1H), 8.61 (d, J=7.7 Hz, 1H), 8.00 (d, J=7.9 Hz, 1H), 7.88-7.79 (m, 3H), 7.75-7.69 (m, 1H), 7.54 (d, J=2.1 Hz, 1H), 7.22-7.15 (m, 2H), 7.14-7.09 (m, 1H), 4.67 (s, 2H), 4.57 (s, 2H), 2.10 (s, 3H), 1.52-1.38 (m, 5H). LCMS (ESI): m/z=464.1 [M+H]⁺.

### Step 2: Preparation of 2-methyl-N-(1-(2-(1-(2-(methylsulfonyl)ethyl)-1H-pyrazol-4-yl)quinolin-4-yl)cyclopropyl-44-((thiazol-4-ylmethoxy)ylmethyl)benzamide

To a mixture of N-(1-(2-chloroquinolin-4-yl)cyclopropyl)-2-methyl-4-((thiazol-4-ylmethoxy)methyl)benzamide (100.0 mg, 0.216 mmol) in dioxane (2.0 mL) and H₂O (1.0 mL) were added 1-(2-(methylsulfonyl)ethyl)-4-(4,4,5,5-tetramethyl-1,3,2-dioxaborolan-2-yl)-1H-pyrazole (77.6 mg, 0.259 mmol), Pd(dppf)Cl₂ (15.8 mg, 0.0216 mmol) and K₃PO₃ (137 mg, 0.647 mmol) at 25° C. The mixture was heated to 100° C. for 3 h then diluted by H₂O (5 mL) and extracted with EtOAc (5 mL×3). The organic layer was evaporated under vacuum to give a crude, which was purified by silica gel chromatography (silica gel 12 g, MeOH/EtOAc=0˜20% 254 nm UV) to give another crude. This crude was purified again via a prep-HPLC to afford 2-methyl-N-(1-(2-(1-(2-(methylsulfonyl)ethyl)-1H-pyrazol-4-yl)quinolin-4-yl)cyclopropyl)-4-((thiazol-4-ylmethoxy)methyl)benzamide (48 mg, 37%) as a white solid after lyophilization. ¹H NMR (400 MHz, DMSO-d₆) δ=9.17 (s, 1H), 9.00 (d, J=1.9 Hz, 1H), 8.60 (s, 1H), 8.50 (d, J=8.3 Hz, 1H), 8.20 (s, 1H), 7.89 (s, 1H), 7.90 (d, J=7.8 Hz, 1H), 7.64 (t, J=7.2 Hz, 1H), 7.56 (d, J=1.9 Hz, 1H), 7.49 (t, J=7.6 Hz, 1H), 7.06 (s, 1H), 7.07 (d, J=7.3 Hz, 1H), 7.03-6.99 (m, 1H), 4.59 (t, J=6.8 Hz, 2H), 4.53 (s, 2H), 4.44 (s, 2H), 3.73 (t, J=6.8 Hz, 2H), 2.86 (s, 3H), 1.99 (s, 3H), 1.29 (br d, J=9.3 Hz, 4H). LCMS (ESI): m/z=602.3 [M+H]⁺.

### Prep-HPLC Condition:

Column: Boston Prime C18 150*30 mm*5 μm;
Mobile phase: water (ammonia hydroxide v/v)-MeCN; B %: 35%-55%,
Flow Rate (mL/min): 35 mL/min.

### Example 329: N-(2-(dimethylamino)ethyl)-N-methyl-5-(4-(1-(2-methyl-4-((thiazol-4-ylmethoxy)methyl)benzamido)cyclopropyl)quinolin-2-yl)-1H-pyrazole-3-carboxamide

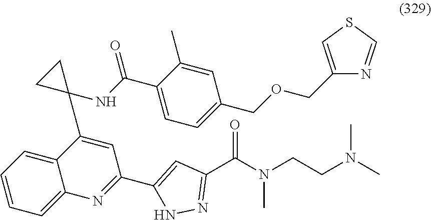 [SMILES: Cc1cc(COCc2cscn2)ccc1C(=O)NC1(c2cc(-c3cc(C(=O)N(C)CCN(C)C)n[nH]3)nc3ccccc23)CC1]

### Step 1: Preparation of N-(1-(2-chloroquinolan-4-ylcyclopropyl)-2-methyl-4-((thiazol-4-ylmethoxy)methyl)benzamide

To a mixture of 1-(2-chloroquinolin-4-yl)cyclopropan-1-amine (1000.0 mg, 4.573 mmol) in DMF (20.0 mL) were added DIEA (1770 mg, 13.7 mmol) and HATU (2610 mg, 6.86 mmol). After stirring for 10 min, 2-methyl-4-((thiazol-4-ylmethoxy)methyl)benzoic acid (1200 mg, 4.57 mmol) was added to the mixture at 25° C. The mixture was stirred at 25° C. for 1 h. After that, the reaction was diluted with water (30 mL) and extracted with EtOAc (30 mL×2). The combined organic phase was washed with brine (30 mL), dried over Na₂SO₄ and evaporated to give a crude, which was purified by chromatography on a silica gel column (20 g silica gel column, PE/EA from 100%:0 to 0%:100%) to give product N-(1-(2-chloroquinolin-4-yl)cyclopropyl)-2-methyl-4-((thiazol-4-ylmethoxy)methyl)benzamide (2100 mg, 99%) as a gum, LCMS (ESI): m/z=464.1 [M+H]⁺.

### Step 2: Preparation of 5-(4-(1-(2-methyl-4-((thiazol-4-ylmethoxy)methylbenzamido)cyclopropyl) quinolin-2-yl)-1-(tetrahydro-2H-pyran-2-yl)-1H-pyrazole-3-carboxylic acid

To a solution of N-(1-(2-chloroquinolin-4-yl)cyclopropyl)-2-methyl-4-((thiazol-4-ylmethoxy)methyl)benzamide (1000.0 mg, 2.155 mmol) in 1,4-dioxane (10.0 mL) and H₂O (3.0 mL) were added methyl 1-(tetrahydro-2H-pyran-2-yl)-5-(4,4,5,5-tetramethyl-1,3,2-dioxaborolan-2-yl)-1H-pyrazole-3-carboxylate (1090 mg, 3.23 mmol), Pd(dppf)C₂ (158 mg, 0.216 mmol) and K₃PO₄ (1370 mg, 6.47 mmol) at 20° C. The mixture was bubbled with N₂ three times and stirred at 100° C. for 16 h. After that, the reaction mixture was evaporated under vacuum to give a crude, which was purified by flash chromatography (20 g silica gel column, MeOH/EtOAc from 0 to 30%) to give 5-(4-(1-(2-methyl-4-((thiazol-4-ylmethoxy)methyl)benzamido)cyclopropyl) quinolin-2-yl)-1-(tetrahydro-2H-pyran-2-yl)-1H-pyrazole-3-carboxylic acid (1000 mg, 74.4%) as a gum. ¹H NMR (400 MHz, DMSO-d₆) δ=9.30 (s, 1H), 9.08 (d, J=2.0 Hz, 1H), 8.68 (d, J=8.4 Hz, 1H), 8.13-8.09 (m, 2H), 7.84 (t, J=7.6 Hz, 1H), 7.76-7.68 (m, 1H), 7.63 (d, J=2.0 Hz, 1H), 7.46 (s, 1H), 7.16-7.13 (m, 2H), 7.10 (d, J=7.8 Hz, 1H), 6.72 (br d, J=8.6 Hz, 1H), 4.61 (s, 2H), 4.52 (s, 2H), 3.99-3.86 (m, 2H), 3.79-3.66 (m, 2H), 3.62 (br d, J=11.2 Hz, 1H), 3.22-3.12 (m, 3H), 2.90 (s, 1H), 2.79-2.69 (m, 1H), 2.01 (br d, J=12.2 Hz, 3H), 1.75 (br s, 1H), 1.63 (br s, 1H), 1.49-1.31 (m, 4H). LCMS (ESI): m/z=646.4 [M+Na]⁺.

### Step 3: Preparation of N-(2-(dimethylamino)ethyl)-N-methyl-5-(4-(1-(2-methyl-4-((thiazol-4-ylmethoxy)methyl)benzamido)cyclopropyl)quinolin-2-yl)-1-(tetrahydro-2H-pyran-2-yl)-1H-pyrazole-3-carboxamide

To a mixture of 5-(4-(1-(2-methyl-4-((thiazol-4-ylmethoxy)methyl)benzamido)cyclopropyl) quinolin-2-yl)-1-(tetrahydro-2H-pyran-2-yl)-1H-pyrazole-3-carboxylic acid (60.0 mg, 0.11 mmol) in DMF (2.0 mL) were added DIEA (43.1 mg, 0.334 mmol) and HATU (63.4 mg, 0.167 mmol). After stirred at 25° C. for 10 min, N1,N1,N2-trimethylethane-1,2-diamine (11.4 mg, 0.111 mmol) was added to the mixture at 25° C. and the mixture was stirred at 25° C. for 1 h. After that, the reaction mixture was quenched with H₂O (5 mL) and extracted with EtOAc (5 mL×3). The organic layer was dried over Na₂SO₄ and evaporated to give N-(2-(dimethylamino)ethyl)-N-methyl-5-(4-(1-(2-methyl-4-((thiazol-4-ylmethoxy)methyl)benzamido)cyclopropyl)quinolin-2-yl)-1-(tetrahydro-2H-pyran-2-yl)-1H-pyrazole-3-carboxamide (150 mg, 100%) as a gum. LCMS (ESI): m/z=708.4 [M+H]⁺.

### Step 4: Preparation of N-(2-(dimethylamino)ethyl)-N-methyl-5-(4-(1-(2-methyl-4-((thiazol-4-ylmethoxy)methyl)benzamido)cyclopropyl)quinolin-2-yl)-1H-pyrazole-3-carboxamide

To a solution of N-(2-(dimethylamino)ethyl)-N-methyl-5-(4-(1-(2-methyl-4-((thiazol-4-ylmethoxy)methyl)benzamido)cyclopropyl)quinolin-2-yl)-1-(tetrahydro-2H-pyran-2-yl)-1H-pyrazole-3-carboxamide (150.0 mg, 0.212 mmol) in CH₂Cl₂ (2.0 mL) was added 4M HCl in dioxane (1.0 mL) at 25° C. The mixture was stirred at 25° C. for 2 h then evaporated to give a crude, which was purified by prep-HPLC to afford N-(2-(dimethylamino)ethyl)-N-methyl-5-(4-(1-(2-methyl-4-((thiazol-4-ylmethoxy)methyl)benzamido)cyclopropyl)quinolin-2-yl)-1H-pyrazole-3-carboxamide (24.3 mg, 18.4%) as a white solid after lyophilization. ¹H NMR (400 MHz, DMSO-d6) δ=13.99 (br s, 1H), 9.27 (s, 1H), 9.08 (d, J=2.0 Hz, 1H), 865 (d, J=8.2 Hz, 1H), 8.27 (br s, 1H), 8.08 (br d, J=8.6 Hz, 1H), 7.79 (br t, J=7.6 Hz, 1H), 7.68-7.61 (m, 2H), 7.36 (br s, 1H), 7.14 (s, 1H), 7.15 (d, J=8.1 Hz, 1H), 7.12-7.08 (m, 1H), 4.61 (s, 2H), 4.52 (s, 2H), 3.85 (br s, 1H), 3.59 (br s, 1H), 3.03 (br s, 1H), 2.22 (s, 3H), 2.13 (br s, 3H), 2.05 (s, 3H), 1.41 (br s, 4H). LCMS (ESI): m/z=624.5 [M+H]⁺.

### Prep-HPLC Condition:

Column: C18 150*40 mm
Mobile phase: water (ammonia hydroxide v/v)-MeCN; B %: 22%-62%,
Flow Rate (mL/min): 60 mL/min.

### Example 330: 4-(((5-(hydroxymethyl)thiazol-4-yl)methoxy)methyl)-2-methyl-N-(1-(2-(1-methyl-1H-pyrazol-4-yl)quinolin-4-yl)cyclopropyl)benzamide

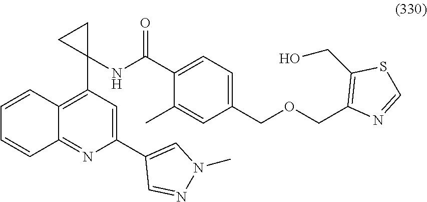 [SMILES: Cc1cc(COCc2ncsc2CO)ccc1C(=O)NC1(c2cc(-c3cnn(C)c3)nc3ccccc23)CC1]

### Step 1: Preparation of 4-(hydroxymethyl)-2-methyl-N-(1-(2-(1-methyl-1H-pyrazol-4-yl)quinolin-4-yl)cyclopropyl)benzamide

To a solution of 4-(hydroxymethyl)-2-methylbenzoic acid (92.0 mg, 0.55 mmol) in DMF (3 mL) were added HATU (316 mg, 0.830 mmol), TEA (0.5 mL) and 1-(2-(1-methyl-1H-pyrazol-4-yl)quinolin-4-yl)cyclopropan-1-amine (117 mg, 0.443 mmol) at 20° C. The reaction mixture was stirred at 20° C. for 15 h then evaporated in vacuo, and purified by silia gel chromatography via a Biotage (20 g silica gel column, PE:EtOAc=1:0 to 0:1, EtOAc: MeOH=1:0 to 0:1) to afford 4-(hydroxymethyl)-2-methyl-N-(1-(2-(1-methyl-1H-pyrazol-4-yl)quinolin-4-yl)cyclopropyl)benzamide (186 mg, 81%) as a yellow solid. LCMS (ESI): m/z=413.2 [M+H]⁺.

### Step 2: Preparation of 4-(((3-methyl-4-((1-(2-(1-methyl-1H-pyrazol-4-yl)quino in-4-yl)cyclopropyl)carbamoyl)benzyl)oxy)methyl)thiazole-5-carboxylic acid

To a solution of 4-(hydroxymethyl)-2-methyl-N-(1-(2-(1-methyl-1H-pyrazol-4-yl)quinolin-4-yl)cyclopropyl)benzamide (150.0 mg, 0.364 mmol) in THF (15 mL) under N₂ was added NaH (21.8 mg, 0.545 mmol) at 0° C. The reaction mixture was stirred at 0° C. under N₂ for 0.5 h before ethyl 4-(bromomethyl)thiazole-5-carboxylate was added to it (136 mg, 0.545 mmol) at 0° C. The reaction mixture was stirred at 20° C. under N₂ for 3 h then quenched with 5 mL 1N HCl at 0° C. The crude reaction mixture was evaporated in vacuo, dissolved in 5 mL DMF and purified by prep-HPLC to afford 4-(((3-methyl-4-((1-(2-(1-methyl-1H-pyrazol-4-yl)quinolin-4-yl)cyclopropyl)carbamoyl)benzyl)oxy)methyl)thiazole-5-carboxylic acid (57 mg, 28%) as a white solid after lyophilization. ¹H NMR (400 MHz, MeOD) 5 ppm 9.23 (s, 1H), 9.18 (s, 1H), 8.57 (d, J=8.0 Hz, 1H), 8.54 (s, 1H), 8.18 (s, 1H), 7.98-7.93 (m, 2H), 7.71 (t, J=7.2 Hz, 1H), 7.55 (t, J=7.6 Hz, 1H), 7.17-7.04 (m, 3H), 4.88 (s, 2H), 4.51 (s, 2H), 3.95 (s, 3H), 2.06 (s, 3H), 1.44-1.28 (m, 4H). LCMS (ESI): m/z=554.3 [M+H].

### Prep-HPLC Condition:

Column: Boston Prime C18 150*30 mm*5 μm
Mobile phase: water (NH3·H2O—NH₄HCO3 v/v)-MeCN; B %: 3%-43%,
Flow Rate (mL/min): 30 mL/min.

### Step 3: Preparation of 4-(((5-(hydroxymethyl)thiazol-4-yl)methoxy)methyl)-2-methyl-N-(1-(2-(1-methyl-1H-pyrazol-4-yl)quinolin-4-yl)cyclopropyl)benzamide

To a solution of 4-(((3-methyl-4-((1-(2-(1-methyl-1H-pyrazol-4-yl)quinolin-4-yl)cyclopropyl)carbamoyl)benzyl)oxy)methyl)thiazole-5-carboxylic acid (100.0 mg, 0.181 mmol) in THF (10 mL) was added LiAlH₄ (1.0 mL, 1 M, 1 mmol) at 0° C. The reaction mixture was then stirred at 20° C. for 2 h before being quenched with 1 mL 1N HCl at 0° C. and filtered. The filtrate was evaporated in vacuo to afford a crude, which was then dissolved in 5 mL DMF and purified by prep-HPLC to afford 4-(((5-(hydroxymethyl)thiazol-4-yl)methoxy)methyl)-2-methyl-N-(1-(2-(1-methyl-1H-pyrazol-4-yl)quinolin-4-yl)cyclopropyl)benzamide (16 mg, 16%) as a white solid. ¹H NMR (400 MHz, METHANOL-d4) δ=8.88 (s, 1H), 8.56 (d, J=7.7 Hz, 1H), 8.41 (s, 1H), 8.27 (s, 1H), 8.10 (s, 1H), 8.07 (d, J=8.4 Hz, 1H), 7.76 (t, J=7.4 Hz, 1H), 7.64-7.59 (m, 1H), 7.18-7.10 (m, 3H), 4.80 (s, 2H), 4.64 (br s, 2H), 4.51 (s, 2H), 4.03 (5, 3H), 2.13-2.06 (m, 3H), 1.53-1.41 (m, 4H). LCMS (ESI): m/z=540.1 [M+H]⁺.

### Prep-HPLC Condition:

Column: Boston Prime C18 150*30 mm*5 μm;
Mobile phase: water (NH3·H2O—NH4HCO3 v/v)-MeCN; B %: 18%-58%,
Flow Rate (mL/min): 30 mL/min.

### Example 342: N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide

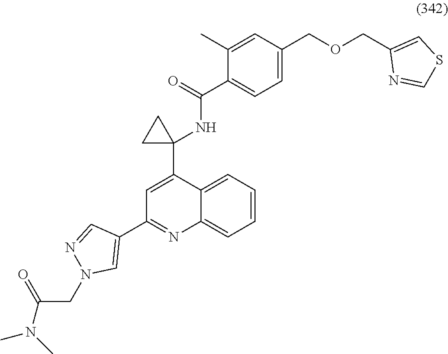 [SMILES: Cc1cc(COCc2cscn2)ccc1C(=O)NC1(c2cc(-c3cnn(CC(=O)N(C)C)c3)nc3ccccc23)CC1]

### Step 1: Preparation of 2-(4-(4-(1-(2-methyl-4-((thiazol-4-ylmethoxy)methyl)benzamido)cyclopropyl)quinolin-2-yl)-1H-pyrazol-1-yl)acetic acid

To a mixture of N-(1-(2-chloroquinolin-4-yl)cyclopropyl)-2-methyl-4-((thiazol-4-ylmethoxy)methyl)benzamide (200.0 mg, 0.345 mmol) and methyl 2-(4-(4,4,5,5-tetramethyl-1,3,2-dioxaborolan-2-yl)-1H-pyrazol-1-yl)acetate (91.5 mg, 0.345 mmol) in dioxane (5.0 mL) were added sat. aq. Na₂CO₃ (100.0 mg, 1.03 mmol) in H₂O (1.0 mL). The mixture was degassed with N₂ for 1 minute, then Pd(dppf)Cl₂ (25.2 mg, 0.0345 mmol) was added to it. The reaction was purged with N₂ for 1 minute the stirred at 100° C. for 18 h. After that, the reaction was evaporated to give a crude, which was purified by chromatography (silica gel 12 g, MeOH/EtOAc from 0% 100% to 30%:70%) to afford 2-(4-(4-(1-(2-methyl-4-((thiazol-4-ylmethoxy)methyl)benzamido)cyclopropy)quinolin-2-yl)-1H-pyrazol-1-yl)acetic acid (95.0 mg, 49.8%) as a yellow oil. LCMS (ESI): m/z=554.3 [M+H]⁺.

### Step 2: Preparation of N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide

To a mixture of 2-(4-(4-(1-(2-methyl-4-((thiazol-4-ylmethoxy)methyl)benzamido)cyclopropyl)quinolin-2-yl)-1H-pyrazol-1-yl)acetic acid (95 mg, 0.172 mmol) in DMF (3.0 mL) were added DIEA (66.5 mg, 0.515 mmol) and HATU (97.9 mg, 0.257 mmol) at 0° C. Then Me₂NH·HCl (14.0 mg, 0.172 mmol) was added to the mixture followed by stirring at 25° C. for 1 h. Me₂NH·HCl (28.0 mg, 0.343 mmol) was added to the reaction followed by stirring at 25° C. for 1 h. HATU (32.6 mg, 0.0858 mmol) was added to the reaction followed by stirring at 25° C. for 17 h. HATU (32.6 mg, 0.0858 mmol) was added to the reaction followed by stirring at 25° C. for 1 h. At which time, LCMS showed starting material disappeared and a product with desired mass was formed. The reaction was then filtered, and the filtrate was purified by HPLC to afford N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide (21.29 mg, 7.12%) as a white solid after lyophilization. ¹H NMR (400 MHz, METHANOL-d4) δ=8.98 (d, J=1.8 Hz, 1H), 8.57-8.52 (m, 1H), 8.43 (s, 1H), 8.30 (s, 1H), 8.11 (s, 1H), 8.09-8.04 (m, 1H), 7.76-7.71 (m, 1H), 7.62-7.57 (m, 1H), 7.52 (s, 1H), 7.15 (br d, J=3.8 Hz, 2H), 7.12 (s, 1H), 5.25 (s, 2H), 4.65 (s, 2H), 4.54 (s, 2H), 3.17 (s, 3H), 3.00 (s, 3H), 2.10 (s, 3H), 1.49 (s, 2H), 1.42 (s, 2H). LCMS (ESI): m/z=581.4 [M+H]⁺.

### Prep-HPLC Condition:

Column: C18 150*40 mm
Mobile phase: water (NH3·H2O+NH₄HCO3)-MeCN: B %: 17%-57%,
Flow Rate (mL/min): 60 mL/min.

### Example 352: N-(1-(2-(1-(2-amino-2-oxoethyl)-1H-pyrazol-4-yl)quinolin-4-yl)cyclopropyl)-2-methyl-4-((thiazol-4-ylmethoxy)methyl)benzamide

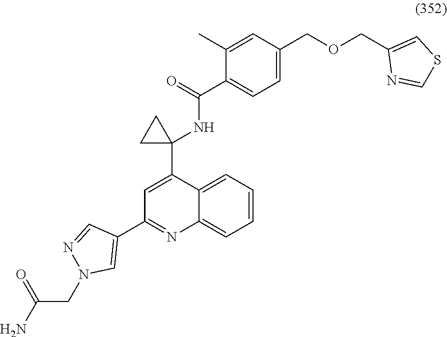 [SMILES: Cc1cc(COCc2cscn2)ccc1C(=O)NC1(c2cc(-c3cnn(CC(N)=O)c3)nc3ccccc23)CC1]

To a mixture of N-(1-(2-chloroquinolin-4-yl)cyclopropyl)-2-methyl-4-((thiazol-4-ylmethoxy)methyl)benzamide (120.0 mg, 0.207 mmol) and 2-(4-(44,5,5-tetramethyl-1,3,2-dioxaborolan-2-yl)-1H-pyrazol-1-yl)acetonitrile (48.2 ng, 0.207 mmol) in dioxane (5.0 mL) were added Na₂CO₃ (65.8 mg, 0.621 mmol) in H₂O (1.0 mL). The mixture was degassed with N₂ for 1 minute then Pd(dppf)Cl₂ (15.1 mg, 0.0207 mmol) was added to it. The mixture was purged with N₂ for 1 minute and stirred at 100° C. for 16 h. After that, the reaction mixture was evaporated and purified by column chromatography (silica gel 12 g, MeOH/EtOAc from 0:100% to 20%:80%, 254 nm UV) to give a crude product (65.0 mg) as a yellow solid. The crude (65.0 ng) was dissolved in MeOH (1.5 mL), filtered, purified by prep-HPLC to afford N-(1-(2-(1-(2-amino-2-oxoethyl)-1H-pyrazol-4-yl)quinolin-4-yl)cyclopropyl)-2-methyl-4-((thiazol-4-ylmethoxy)methyl)benzamide (17.1 mg, 15.0%) as a white solid after lyophilization. ¹H NMR (400 MHz, DMSO-d6) δ=9.23 (s, 1H), 9.07 (d, J=1.9 Hz, 1H), 8.57 (d, J=8.3 Hz, 1H), 8.53 (s, 1H), 8.20 (s, 1H), 7.96 (s, 2H), 7.73-7.68 (m, 1H), 7.62 (d, J=1.6 Hz, 1H), 7.59-7.53 (m, 2H), 7.35-7.30 (m, 1H), 7.13 (s, 2H), 7.09 (s, 1H), 4.86 (s, 2H), 4.60 (s, 2H), 4.51 (s, 2H), 2.05 (s, 3H), 1.37 (br s, 2H), 1.35 (br s, 2H). LCMS (ESI): m/z=553.3 [M+H]⁺.

### Example 353: 4-(2-methoxy-1-(thiazol-4-ylmethoxy)ethyl)-2-methyl-N-(1-(2-methyl-7-(1-methyl-1H-pyrazol-4-yl)quinolin-5-yl)cyclopropyl)benzamide

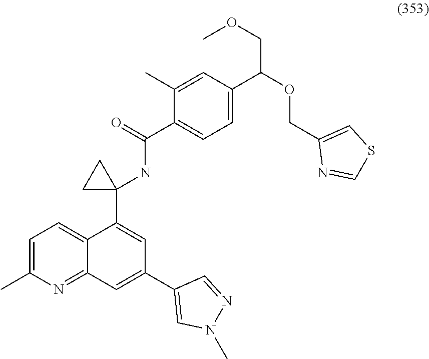 [SMILES: COCC(OCc1cscn1)c1ccc(C(=O)NC2(c3cc(-c4cnn(C)c4)cc4nc(C)ccc34)CC2)c(C)c1]

### Step 1: Preparation of 1-(2-methyl-7-(1-methyl-1H-pyrazol-4-yl)quinolin-5-yl)cyclopropan-1-amine

To a solution of 5-(1-aminocyclopropyl)-2-methylquinolin-7-yl trifluoromethanesulfonate (350 mg, 1.01 mmol) in 1,4-dioxane (8.0 mL) and H₂O (2.0 mL) were added 1-methyl-4-(4,4,5,5-tetramethyl-1,3,2-dioxaborolan-2-yl)-1H-pyrazole (421 mg, 2.02 mmol), Pd(dppf)Cl₂ (73.9 mg, 0.101 mmol) and K₃PO₄ (644 mg, 3.03 mmol) at 20° C. The mixture was bubbled with N for 3 times and stirred at 100° C. for 16 h. The reaction mixture was then partitioned with DCM (10 mL) and H₂O (10 mL). The aqueous phase was extracted with DCM (10 mL×3). The combined organic layer was evaporated under vacuum to give a crude, which was purified by flash chromatography (12 g silica gel column, MeOH/DCM from 0 to 10%) to give 1-(2-methyl-7-(1-methyl-1H-pyrazol-4-yl)quinolin-5-yl)cyclopropan-1-amine (220 mg, 78.2%) as a brown gum.

LCMS (ESI): m/z=279.2 [M+H]⁺.

### Step 2: Preparation of 4-(2-methoxy-1-(thiazol-4-ylmethoxy)ethyl)-2-methyl-N-(1-(2-methyl-7-(1 methyl-1H-pyrazol-4-yl)quinolin-5-yl)cyclopropyl)benzamide

Using a procedure similar to Example 324 step 7 and starting from 4-(2-methoxy-1-(thiazol-4-ylmethoxy)ethyl)-2-methylbenzoic acid and 1-(2-methyl-7-(1-methyl-1H-pyrazol-4-yl)quinolin-5-yl)cyclopropan-1-amine afford 4-(2-methoxy-1-(thiazol-4-ylmethoxy)ethyl)-2-methyl-N-(1-(2-methyl-7-(1-methyl-1H-pyrazol-4-yl)quinolin-5-yl)cyclopropyl)benzamide. ¹H NMR (400 MHz, METHANOL-d4) δ=9.00-3.91 (m, 2H), 3.24 (s, 1H), 313 (d, J=1.5 Hz, 1H), 8.07 (s, 1H), 3.03 (s, 1H), 7.55-7.50 (m, 1H), 7.46 (d, J=3.6 Hz, 1H), 7.21-7.17 (m, 2H), 7.15-7.10 (m, 1H), 4.62 (dd, J=4.0, 7.3 Hz, 1H), 4.56 (d, J=3.5 Hz, 2H), 4.00 (s, 3H), 360 (dd, J=7.4, 10.6 Hz, 1H), 3.47-3.41 (m, 1H), 3.33 (br s, 3H), 2.76 (s, 3H), 2.13 (s, 3H), 1.53-1.35 (in, 4H). LCMS (ESI): m/z=568 [M+H]⁺.

### Example 354: N-(1-(2-(2-hydroxypropan-2-yl)-7-(1-methyl-1H-pyrazol-4-yl)quinolin-5-yl)cyclopropyl)-2-methyl-4-((thiazol-4-ylmethoxy)methyl)benzamide

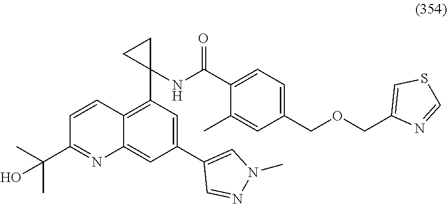 [SMILES: Cc1cc(COCc2cscn2)ccc1C(=O)NC1(c2cc(-c3cnn(C)c3)cc3nc(C(C)(C)O)ccc23)CC1]

### Step 1: Preparation of N-(L-(2-cyano-7-(1-methyl-1H-pyrazol-4-yl)quinolin-5-yl)cyclopropyl)-2-methyl-4-((thiazol-4-ylmethoxy)methyl)benzamide

To a solution of 7-(1-methyl-1H-pyrazol-4-yl)-5-(1-(2-methyl-4-((thiazol-4-ylmethoxy)methyl)benzamido)cyclopropyl)quinoline 1-oxide (200 mg, 0.381 mmol) in DCE (5.00 mL) was added TMSCN (49 mg, 0.49 mmol) and dimethylcarbamyl chloride (40.9 mg, 0.381 mmol) at 25° C.

Then the mixture was stirred at 60° C. for 21 h. LCMS showed that the starting material was consumed and a major peak (˜90%) with a desired mass value (m/z=535.3, [M+H]⁺) was observed. The reaction mixture was evaporated in vacuo to give a residual, which was then diluted by H₂O (20 mL) and extracted with EtOAc (15 mL×3). The combined organic layer was washed with brine (30 mL), dried over Na₂SO₄ and evaporated in vacuo to give a crude, which was then purified by flash chromatography (4 g silica gel column, EtOAc/petroleum ether from 0 to 100%) to give N-(1-(2-cyano-7-(1-methyl-1H-pyrazol-4-yl)quinolin-5-yl)cyclopropyl)-2-methyl-4-((thiazol-4-ylmethoxy)methyl)benzamide (150 mg, 73.7%) as a brown solid. LCMS (ESI): m/z=535.2, [M+H]⁺, Step 2: Preparation of N-(1-(2-acetyl-7-(1-methyl-1H-pyrazol-4-yl)quinolin-5-yl)cyclopropyl)-2-methyl-4-((thiazol-4-ylmethoxy)methyl)benzamide

To a mixture of N-(1-(2-cyano-7-(1-methyl-H-pyrazol-4-yl)quinolin-5-yl)cyclopropyl)-2-methyl-4-((thiazol-4-ylmethoxy)methyl)benzamide (150.0 mg, 0.281 mmol) in THF (4.00 mL) was added MeMgBr in diethyl ether (0.1 mL, 0.30 mmol) slowly at −10° C. under N₂. The reaction was stirred for 1 h at −10° C. then slowly warmed up to 25° C. and stirred for 3 h. LCMS showed 92% of the starting material remained and no desired was observed. To the reaction mixture at −10° C. was then added MeMgBr in diethyl ether (0.2 mL, 0.60 mmol). The reaction mixture was slowly warmed up to 25° C. and stirred for 16 h. LCMS showed 43% of the starting material remained and a product with a desired mass was formed (47%, m/z=552.1, [M+H]⁺). To the reaction mixture at −10° C. was added MeMgBr in diethyl ether (0.2 mL, 0.60 mmol). The reaction mixture was slowly warmed up to 25° C. and stirred for 2 h. LCMS showed the starting material was consumed and the product with desired mass was formed at ˜88%. The reaction was then diluted by H₂O (20 mL) at 25° C. and extracted with EtOAc (15 mL×3). The combined organic layer was washed with brine (20 mL), dried over Na₂SO₄ and evaporated in vacuo to give a crude, which was purified by flash chromatography (4 g silica gel column, EtOAc/petroleum ether from 0 to 100%) to give N-(1-(2-acetyl-7-(1-methyl-1H-pyrazol-4-yl)quinolin-5-yl)cyclopropyl)-2-methyl-4-((thiazol-4-ylmethoxy)methyl)benzamide (65 mg, 42.0%) as a yellow gum. LCMS (ESI): m/z=552.1 [M+H]⁺.

### Step 3: Preparation of N-(1-(2-(2-hydroxypropan-2-vi)-7-(1-methyl-1H-pyrazol-4-yl)quinolin-5-yl)cyclopropyl)-2-methy-4-((thiazol-4-ylmethoxy)methyl)benzamide

To a mixture of N-(1-(2-acetyl-7-(1-methyl-1H-pyrazol-4-yl)quinolin-5-yl)cyclopropyl)-2-methyl-4-((thiazol-4-ylmethoxy)methyl)benzamide (65.0 mg, 0.12 mmol) in THF (3.0 mL) at −10° C. under N₂ was slowly added MeMgBr in diethyl ether (0.08 mL, 0.24 mmol). The reaction mixture was stirred at −10° C. for 1 h then slowly warmed Lip to 25° C. and stirred for 3 h. To the reaction mixture at −10° C. under N₂ was slowly added MeMgBr in diethyl ether (0.08 mL, 0.24 mmol). The reaction mixture stirred at −10° C. for 1 h then slowly warmed up to 25° C. and stirred for 2 h. LCMS showed 47% of the starting material remained and a peak with desired mass was formed (25%, m/z=568.3, [M+H]⁺). The reaction mixture was stirred at 50° C. for 16 h. LCMS showed 40% of the starting material remained and the desired product stayed at 25%. To the reaction mixture at −10° C. under N₂ was slowly added MeMgBr in THF (0.08 mL, 0.24 mmol). The reaction mixture stirred at −10° C. for 1 h then slowly warmed up to 25° C. and stirred for 2 h. LCMS showed 34% of the starting material remained and the desired product increased to 32%. The reaction mixture was quenched by sat. aq. NH₄Cl (10 mL) and extracted with EtOAc (10 mL×2). The combined organic layer was dried over Na₂SO₄ and evaporated in vacuo to give a crude, which was dissolved with DMF purified by prep-HPLC to afford N-(1-(2-(2-hydroxypropan-2-yl)-7-(1-methyl-1H-pyrazol-4-yl)quinolin-5-yl)cyclopropyl)-2-methyl-4-((thiazol-4-ylmethoxy)methyl)benzamide (7.30 mg, 11.0%) as a white solid. ¹H NMR (400 MHz, DMSO-d6) δ=9.17-9.06 (m, 2H), 9.02-8.95 (m, 1H), 8.40 (s, 1H), 8.08 (s, 1H), 8.06-7.98 (m, 2H), 7.80 (d, J=8.9 Hz, 1H), 7.68-7.59 (m, 1H), 7.17-7.03 (m, 3H), 5.44 (s, 1H), 4.60 (s, 2H), 4.51 (s, 2H), 3.91 (s, 3H), 2.09 (s, 3H), 1.55 (s, 6H), 1.36 (br s, 2H), 1.28 (br s, 2H). LCMS (ESI): m/z=568.3 [M+H]⁺.

### Prep-HPLC Condition:

Column: C18 150×40 mm
Mobile phase: water (NH₃·H₂O+NH₄HCO₃)-MeCN; B %: 20%-60%,
Flow Rate (mL/min): 60 mL/min.

### Example 358: N-(1-(2-hydroxy-7-(1-methyl-1H-pyrazol-4-yl)quinolin-5-yl)cyclopropyl)-2-methyl-4-((thiazol-4-ylmethoxy)methyl)benzamide

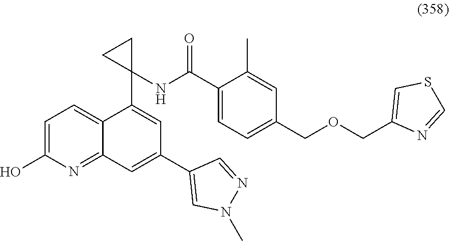 [SMILES: Cc1cc(COCc2cscn2)ccc1C(=O)NC1(c2cc(-c3cnn(C)c3)cc3nc(O)ccc23)CC1]

### Step Preparation of 2-methyl-N-(1-(7-(1-methyl-1H-pyrazol-4-yl)quinolin:5-yl)cyclopropyl)-4-((thiazol-4-ylmethoxy)methyl)benzamide

2-methyl-N-(1-(7-(I-methyl-1H-pyrazol-4-yl)quinolin-5-yl)cyclopropyl)-4-((thiazol-4-ylmethoxy)methyl)benzamide was prepared using a procedure similar to step 6 of Example 274 from 1-(7-(1-methyl-1H-pyrazol-4-yl)quinolin-5-yl)cyclopropan-1-amine and 2-methyl-4-((thiazol-4-ylmethoxy)methyl)benzoic acid. LCMS (ESI): m/z=510.2 [M+H]⁺.

### Step 2: Preparation of 7-(1-methyl-1H-pyrazol-4-yl)-5-(1-(2-methyl-4-((thiazol-4-ylmethoxy)methyl)benzamido)cyclopropyl)quinoline 1-oxide

7-(1-methyl-1H-pyrazol-4-yl)-5-(1-(2-methyl-4-((thiazol-4-ylmethoxy)methyl)benzamido)cyclopropyl)quinoline 1-oxide was prepared using a procedure similar to the preparation P2 from 2-methyl-N-(1-(7-(1-methyl-1H-pyrazol-4-yl)quinolin-5-yl)cyclopropyl)-4-((thiazol-4-ylmethoxy)methyl)benzamide. LCMS (ESI): m/z=526.2 [M+H]⁺.

### Step 3: Preparation of N-(1-(2-hydroxy-7-(1-methyl-1H-pyrazol-4-yl)quinolin-5-yl)cyclopropyl)-2-methyl-4-((thiazol-4-ylmethoxy)methyl)benzamide

To the solution of 7-(1-methyl-1H-pyrazol-4-yl)-5-(1-(2-methyl-4-((thiazol-4-ylmethoxy)methyl)benzamido)cyclopropyl)quinoline 1-oxide (80.0 mg, 0.15 mmol) in DMF (2.0 mL) was added trifluoroacetic anhydride (160 mg, 0.761 mmol) at 0° C., The reaction mixture slowly returned to 25° C. and it was stirred for 16 h. LCMS showed the starting material was consumed and peaks with desired mass were observed (m/z=526.2, [M+H]⁺) and its CF₃CO ester. To the reaction mixture was then added sat. aq. NaHCO₃ (2 mL), then the mixture was stirred at 25° C. for 2 h. LCMS showed a major peak with desired mass (m/z=526.2). The reaction was then combined with another batch (30.0 mg, 0.057 mmol) for workup. The reaction was extracted with EtOAc (10 mL×3) after being diluted by H₂O (10 mL). The combined organic layer was washed with brine (20 mL), dried over Na₂SO₄ and evaporated to give a crude, which was dissolved with DMF and purified by prep-HPLC to afford N-(1-(2-hydroxy-7-(1-methyl-1H-pyrazol-4-yl)quinolin-5-yl)cyclopropyl)-2-methyl-4-((thiazol-4-ylmethoxy)methyl)benzamide (9.77 mg, 9%) as a white solid. ¹H NMR (400 MHz, DMSO-d₆) δ=11.70 (br s, 1H), 9.19-8.96 (m, 2H), 8.58 (d, J=9.8 Hz, 1H), 8.20 (s, 1H), 7.83 (s, 1H), 7.62 (dd, J=1.7, 6.3 Hz, 2H), 7.33 (d, J=1.0 Hz, 1H), 7.26-7.02 (m, 3H), 6.48 (d, J=9.8 Hz, 1H), 4.60 (s, 2H), 4.51 (s, 2H), 390 (s, 3H), 2.12 (s, 3H), 1.35-1.16 (m, 4H). LCMS (ESI): m/z=526.3 [M+H]⁺.

### Prep-HPLC Condition:

Column: C18 150×40 mm
Mobile phase: water (NH3·H2O+NH₄HCO3)-MeCN; B %: 11%-51%,
Flow Rate (mL/min): 60 mL/rnin.

### Example 366: N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide

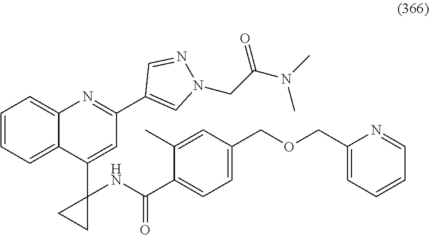 [SMILES: Cc1cc(COCc2ccccn2)ccc1C(=O)NC1(c2cc(-c3cnn(CC(=O)N(C)C)c3)nc3ccccc23)CC1]

### Step 1: Preparation of 2-methyl-4 (2pyridin-ylmethoxy)methyl)benzoic acid

2-methyl-4-((pyridin-2-ylmethoxy)methyl)benzoic acid was prepared using a procedure similar to step 4-5 of Example 274 with methyl 4-(chloromethyl)-2-methylbenzoate and pyridin-2-ylmethanol. LCMS (ESI): m/z=258.2 [M+H]⁺.

### Step 2: Preparation of N-(1-(2-chloroquinolin-4-yl)cyclopropyl)-2-methyl-4-((pyridin-2-ylmethoxy)methyl)benzamide

N-(1-(2-chloroquinolin-4-yl)cyclopropyl)-2-methyl-4-((pyridin-2-ylmethoxy)methyl)benzamide was prepared using a procedure similar to step 1 of Example 328 with 2-methyl-4-((pyridin-2-ylmethoxy)methyl)benzoic acid and 1-(2-chloroquinolin-4-yl)cyclopropan-1-amine. LCMS (ESI): m/z=458.2 [M+H]⁺.

### Step 2: Preparation of 2-(4-(4-(1-(2-methyl-4-((pyridin-2-ylmethoxy)methyl)benzamido)cyclopropyl)quinolin-2-yl)-1H-pyrazol-1-yl)acetic acid

2-(4-(4-(1-(2-methyl-4-((pyridin-2-ylmethoxy)methyl)benzamido)cyclopropyl)quinolin-2-yl)-1H-pyrazol-1-yl)acetic acid was prepared using a procedure similar to step 1 of example 342 with N-(1-(2-chloroquinolin-4-yl)cyclopropyl)-2-methyl-4-((pyridin-2-ylmethoxy)methyl)benzamide and methyl 2-(4-(4,4,5,5-tetramethyl-1,3,2-dioxaborolan-2-yl)-1H-pyrazol-1-yl)acetate. ¹H NMR (400 MHz, DMSO-d₆) δ=9.25 (s, 1H), 8.58 (d, J=8.4 Hz, 1H), 8.55-8.49 (m, 2H), 8.17 (s, 1H), 7.99-7.94 (m, 2H), 7.80 (dt, J=1.8, 7.7 Hz, 1H), 7.74-7.67 (m, 1H), 7.56 (t, J=7.0 Hz, 1H), 7.48-7.40 (m, 1H), 7.33-7.27 (m, 1H), 7.19-7.14 (m, 2H), 7.13-7.08 (m, 1H), 4.90 (s, 2H), 4.56 (d, J=5.9 Hz, 4H), 2.07 (s, 3H), 1.37 (br d, J=7.7 Hz, 4H). LCMS (ESI): m/z=548.2 [M+H]⁺.

### Step 3: Preparation of N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide

N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide was prepared using a procedure similar to step 2 of Example 342 with 2-(4-(4-(1-(2-methyl-4-((pyridin-2-ylmethoxy)methyl)benzamido)cyclopropyl) quinolin-2-yl)-1H-pyrazol-1-yl)acetic acid and dimethylamine. ¹H NMR (400 MHz, DMSO-d₆) δ=9.24 (s, 1H), 8.58 (d, J=8.1 Hz, 1H), 8.51 (d, J=4.1 Hz, 1H), 8.48 (s, 1H), 8.19 (s, 1H), 7.97 (s, 1H), 7.97 (d, J=7.6 Hz, 1H), 7.80 (dt, J=1.8, 7.6 Hz, 1H), 7.71 (t, J=7.6 Hz, 1H), 7.56 (t, J=7.1 Hz, 1H), 7.45 (d, J=7.8 Hz, 1H), 7.30 (dd, J=4.8, 7.1 Hz, 1H), 7.19-7.14 (m, 2H), 7.13-7.08 (m, 1H), 5.22 (s, 2H), 4.56 (d, J=5.8 Hz, 4H), 3.08 (s, 3H), 2.89 (s, 3H), 2.07 (s, 3H), 1.37 (br d, J=4.6 Hz, 4H). LCMS (ESI): m/z=575.4 [M+H]⁺.

### Prep-HPLC Condition:

Column: C18 150*30*5 μm
Mobile phase: water (ammonium hydroxide v/v)-MeCN; B %: 33%-53%,
Flow Rate (mL/min): 35 mL/min.

The following examples were prepared using a protocol described herein:

| | | Procedure and | |
|---|---|---|---|
| Ex. # | Structure; Compound Name | reactants used | Characterizing Data |
| Ex. 331 | 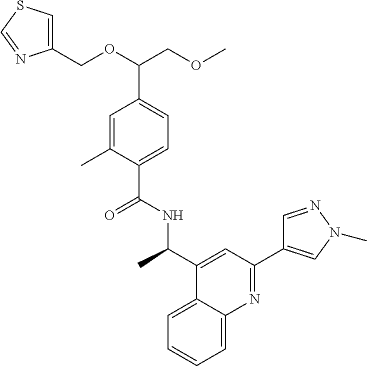 [SMILES: COCC(OCc1cscn1)c1ccc(C(=O)N[C@H](C)c2cc(-c3cnn(C)c3)nc3ccccc23)c(C)c1] | Example 324, step 7 using rac-(R)-4- (2-methoxy-1- (thiazol-4- ylmethoxy)ethyl)-2- methylbenzoic acid and (R)-1-(2-(1- methyl-1H-pyrazol- 4-yl)quinolin-4- yl)ethan-1-amine. Purification by prep-HPLC followed by an SFC | ¹H NMR (400 MHz, DMSO-d6) δ = 9.06 (s, 1H), 8.98 (br d, J = 7.7 Hz, 1H), 8.41 (s, 1H), 8.23 (br d, J = 8.3 Hz, 1H), 8.09 (s, 1H), 7.97 (d, J = 8.4 Hz, 1H), 7.86 (s, 1H), 7.73 (t, J = 7.6 Hz, 1H), 7.68-7.51 (m, 2H), 7.42-7.32 (m, 1H), 7.31- 7.17 (m, 2H), 5.89 (s, 1H), 4.72- 4.61 (m, 1H), 4.51 (s, 2H), 4.44- 4.37 (m, 1H), 3.94 (s, 3H), 3.60- 3.48 (m, 1H), 3.42 (br dd, J = 4.5, 10.6 Hz, 1H), 3.28-3.15 (m, 3H), 2.33-2.23 (m, 3H), 1.61 (br d, J = 6.8 Hz, 3H). LCMS (ESI): m/z = 542.4 [M + H]⁺. |
| | 4-(2-methoxy-1-(thiazol-4- | | |
| | ylmethoxy)ethyl)-2-methyl- | | |
| | N-((R)-1-(2-(1-methyl-1H- | | |
| | pyrazol-4-yl)quinolin-4- | | |
| | yl)ethyl)benzamide | | |
| Ex. 332 | 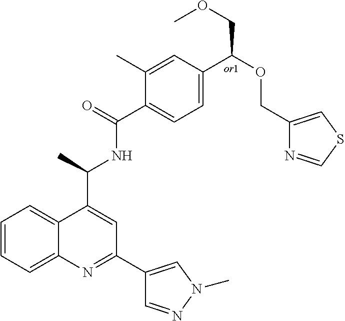 [SMILES: COC[C@@H](OCc1cscn1)c1ccc(C(=O)N[C@H](C)c2cc(-c3cnn(C)c3)nc3ccccc23)c(C)c1] | Example 324, step 7 and step 8 using rac-(R)-4-(2- methoxy-1-(thiazol- 4-ylmethoxy)ethyl)- 2-methylbenzoic acid and (R)-1-(2- (1-methyl-1H- pyrazol-4- yl)quinolin-4- yl)ethan-1-amine | ¹H NMR (400 MHz, DMSO-d6) δ 9.06 (d, J = 1.98 Hz, 1H), 8.98 (d, J = 7.70 Hz, 1H), 8.42 (s, 1H), 8.23 (d, J = 8.14 Hz, 1H), 8.09 (s, 1H), 7.97 (d, J = 7.70 Hz, 1H), 7.86 (s, 1H), 7.73 (t, J = 7.04 Hz, 1H), 7.55- 7.62 (m, 2H), 7.37 (d, J = 7.70 Hz, 1H), 7.24-7.29 (m, 2H), 5.88 (t, J = 7.37 Hz, 1H), 4.67 (dd, J = 4.40, 7.04 Hz, 1H), 4.51 (s, 2H), 3.94 (s, 3H), 3.56 (dd, J = 7.04, 10.56 Hz, 1H), 3.43 (d, J = 4.40 Hz, 1H), 3.32- 3.33 (m, 1H), 3.25 (s, 3H), 2.52- 2.55 (m, 1H), 2.29 (s, 3H), 1.61 (d, J = 7.04 Hz, 3H). LCMS (ESI): m/z = 542.3 [M + H]⁺. |
| | 4-((R*)-2-methoxy-1- | | |
| | (thiazol-4- | | |
| | ylmethoxy)ethyl)-2-methyl- | | |
| | N-((R)-1-(2-(1-methyl-1H- | | |
| | pyrazol-4-yl)quinolin-4- | | |
| | yl)ethyl)benzamide, ENT-1 | | |
| Ex. 333 | 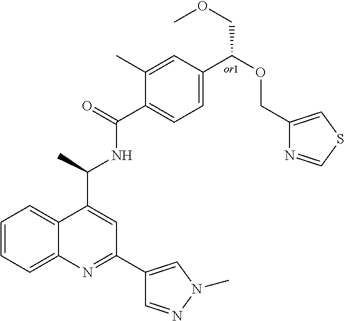 [SMILES: COC[C@H](OCc1cscn1)c1ccc(C(=O)N[C@H](C)c2cc(-c3cnn(C)c3)nc3ccccc23)c(C)c1] | Same as Ex. 332 | ¹H NMR (400 MHz, DMSO-d6) δ 9.06 (d, J = 1.98 Hz, 1H), 8.98 (d, J = 7.92 Hz, 1H), 8.41 (s, 1H), 8.23 (d, J = 8.14 Hz, 1H), 8.09 (s, 1H), 7.97 (d, J = 8.36 Hz, 1H), 7.86 (s, 1H), 7.73 (t, J = 7.59 Hz, 1H), 7.54- 7.63 (m, 2H), 7.37 (d, J = 7.70 Hz, 1H), 7.19-7.30 (m, 2H), 5.88 (t, J = 7.15 Hz, 1H), 4.63-4.69 (m, 1H), 4.51 (s, 2H), 3.94 (s, 3H), 3.56 (dd, J = 7.04, 10.56 Hz, 1H), 3.41-3.45 (m, 1H), 3.25 (s, 3H), 2.29 (s, 3H), 1.61 (d, J = 7.04 Hz, 3H). LCMS (ESI): m/z = 542.3 [M + H]⁺. |
| | 4-((R*)-2-methoxy-1- | | |
| | (thiazol-4- | | |
| | ylmethoxy)ethyl)-2-methyl- | | |
| | N-((R)-1-(2-(1-methyl-1H- | | |
| | pyrazol-4-yl)quinolin-4- | | |
| | yl)ethyl)benzamide, ENT-2 | | |
| Ex. 334 | 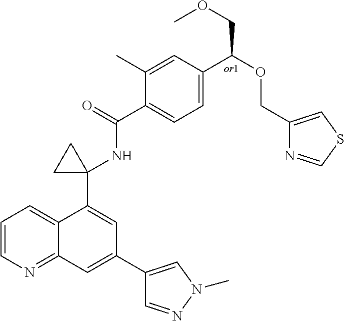 [SMILES: COC[C@@H](OCc1cscn1)c1ccc(C(=O)NC2(c3cc(-c4cnn(C)c4)cc4ncccc34)CC2)c(C)c1] | Example 324, step 7 and step 8 using rac-(R)-4-(2- methoxy-1-(thiazol- 4-ylmethoxy)ethyl)- 2-methylbenzoic acid and 1-(7-(1- methyl-1H-pyrazol- 4-yl)quinolin-5- yl)cyclopropan-1- amine | ¹H NMR (400 MHz, DMSO-d6) δ = 9.17 (s, 1H), 9.05 (d, J = 1.9 Hz, 1H), 8.99 (d, J = 8.3 Hz, 1H), 8.88 (d, J = 2.9 Hz, 1H), 8.41 (s, 1H), 8.12-8.04 (m, 3H), 7.60-7.57 (m, 1H), 7.51 (dd, J = 4.2, 8.4 Hz, 1H), 7.18-7.13 (m, 2H), 7.07 (d, J = 8.1 Hz, 1H), 4.61 (dd, J = 4.4, 6.9 Hz, 1H), 4.47 (s, 2H), 3.93 (s, 3H), 3.51 (dd, J = 7.1, 10.5 Hz, 1H), 3.39-3.35 (m, 1H), 3.25-3.21 (m, 3H), 2.07 (s, 3H), 1.41-1.26 (m, 4H). LCMS (ESI): m/z = 554.3 [M + H]⁺. |
| | rel-(R)-4-(2-methoxy-1- | | |
| | (thiazol-4- | | |
| | ylmethoxy)ethyl)-2-methyl- | | |
| | N-(1-(7-(1-methyl-1H- | | |
| | pyrazol-4-yl)quinolin-5- | | |
| | yl)cyclopropyl)benzamide, | | |
| | ENT-1 | | |
| Ex. 335 | 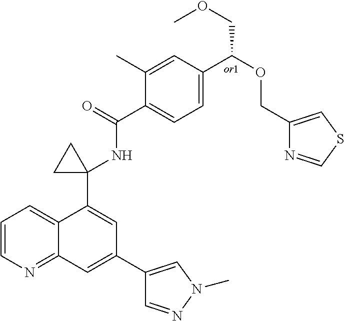 [SMILES: COC[C@H](OCc1cscn1)c1ccc(C(=O)NC2(c3cc(-c4cnn(C)c4)cc4ncccc34)CC2)c(C)c1] | Same as Ex. 334 | ¹H NMR (400 MHz, DMSO-d6) δ = 9.17 (s, 1H), 9.05 (d, J = 2.0 Hz, 1H), 9.02-8.96 (m, 1H), 8.88 (dd, J = 1.5, 4.1 s Hz, 1H), 8.41 (s, 1H), 8.10-8.05 (m, 3H), 7.59 (d, J = 1.8 Hz, 1H), 7.53-7.48 (m, 1H), 7.18-7.13 (m, 2H), 7.07 (d, J = 8.4 Hz, 1H), 4.64-4.57 (m, 1H), 4.51-4.44 (m, 2H), 3.93 (s, 3H), 3.54-3.48 (m, 1H), 3.39-3.34 (m, 1H), 3.22 (s, 3H), 2.09-2.04 (m, 3H), 1.40-1.26 (m, 4H). LCMS (ESI): m/z = 554.4 [M + H]⁺. |
| | rel-(R)-4-(2-methoxy-1- | | |
| | (thiazol-4- | | |
| | ylmethoxy)ethyl)-2-methyl- | | |
| | N-(1-(7-(1-methyl-1H- | | |
| | pyrazol-4-yl)quinolin-5- | | |
| | yl)cyclopropyl)benzamide, | | |
| | ENT-2 | | |
| Ex. 336 | 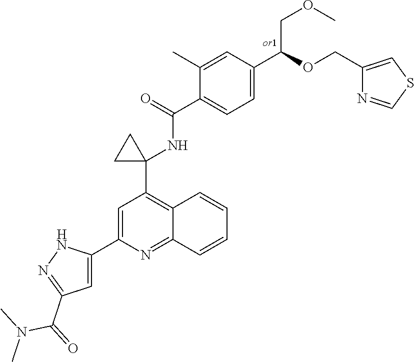 [SMILES: COC[C@@H](OCc1cscn1)c1ccc(C(=O)NC2(c3cc(-c4cc(C(=O)N(C)C)n[nH]4)nc4ccccc34)CC2)c(C)c1] | Example 324, step 7 and step 8 using rac-(R)-4-(2- methoxy-1-(thiazol- 4-ylmethoxy)ethyl)- 2-methylbenzoic acid and 5-(4-(1- aminocyclopropyl) quinolin-2-yl)-N,N- dimethyl-1H- pyrazole-3- carboxamide | ¹H NMR (400 MHz, METHANOL- d4) δ = 8.93 (d, J = 2.0 Hz, 1H), 8.62-8.57 (m, 1H), 8.56-8.19 (m, 1H), 8.16 (br d, J = 8.5 Hz, 1H), 7.79 (t, J = 7.3 Hz, 1H), 7.69-7.63 (m, 1H), 7.52-7.49 (m, 1H), 7.48- 7.35 (m, 1H), 7.17 (s, 2H), 7.14- 7.10 (m, 1H), 4.63-4.60 (m, 1H), 4.51 (s, 1H), 3.62-3.56 (m, 1H), 3.45-3.41 (m, 1H), 3.41-3.38 (m, 3H), 3.32-3.31 (m, 3H), 3.16 (s, 3H), 2.09 (s, 3H), 1.54-1.49 (m, 2H), 1.48-1.43 (m, 2H). LCMS (ESI): m/z = 611.4, [M + H]⁺. |
| | rel-(R)-5-(4-(1-(4-(2- | | |
| | methoxy-1-(thiazol-4- | | |
| | ylmethoxy)ethyl)-2- | | |
| | methylbenzamido)cyclo- | | |
| | propyl)quinolin-2-yl)-N,N- | | |
| | dimethyl-1H-pyrazole-3- | | |
| | carboxamide, ENT-1 | | |
| Ex. 337 | 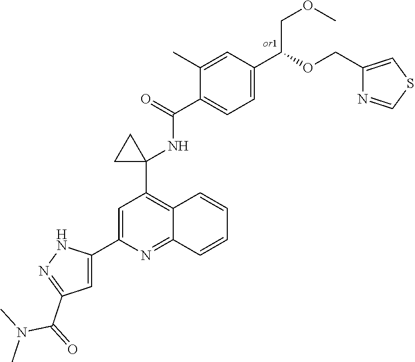 [SMILES: COC[C@H](OCc1cscn1)c1ccc(C(=O)NC2(c3cc(-c4cc(C(=O)N(C)C)n[nH]4)nc4ccccc34)CC2)c(C)c1] | Same as Ex. 336 | ¹H NMR (400 MHz, METHANOL- d4) δ = 8.93 (d, J = 2.1 Hz, 1H), 8.61-8.57 (m, 1H), 8.56-8.19 (m, 1H), 8.19-8.13 (m, 1H), 7.79 (s, 1H), 7.66 (br t, J = 7.6 Hz, 1H), 7.52-7.49 (m, 1H), 7.49-7.34 (m, 1H), 7.17 (s, 2H), 7.14-7.10 (m, 1H), 4.61 (br d, J = 4.3 Hz, 1H), 4.54 (d, J = 3.8 Hz, 2H), 3.59 (dd, J = 7.3, 10.6 Hz, 1H), 3.44 (br d, J = 4.0 Hz, 1H), 3.40 (s, 3H), 3.34- 3.32 (m, 3H), 3.16 (s, 3H), 2.09 (s, 3H), 1.54-1.50 (m, 2H), 1.47- 1.43 (m, 2H). LCMS (ESI): m/z = 611.4, [M + H]⁺. |
| | rel-(R)-5-(4-(1-(4-(2- | | |
| | methoxy-1-(thiazol-4- | | |
| | ylmethoxy)ethyl)-2- | | |
| | methylbenzamido)cyclo- | | |
| | propyl)quinolin-2-yl)-N,N- | | |
| | dimethyl-1H-pyrazole-3- | | |
| | carboxamide, ENT-2 | | |
| Ex. 338 | 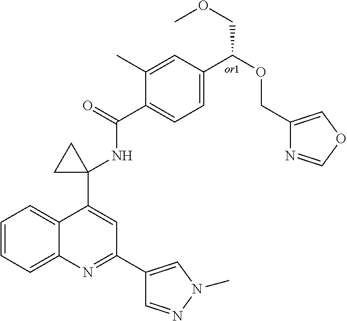 [SMILES: COC[C@H](OCc1cocn1)c1ccc(C(=O)NC2(c3cc(-c4cnn(C)c4)nc4ccccc34)CC2)c(C)c1] | Example 324, step 5-8 using 4- (chloromethyl) oxazole instead of 4-(chloromethyl) thiazole in step 5 | ¹H NMR (400 MHz, DMSO-d6) δ = 9.25 (s, 1H), 8.56 (d, J = 8.4 Hz, 1H), 8.54 (s, 1H), 8.32 (s, 1H), 8.18 (s, 1H), 8.03 (d, J = 0.9 Hz, 1H), 7.98-7.92 (m, 2H), 7.71 (t, J = 7.2 Hz, 1H), 7.55 (t, J = 7.2 Hz, 1H), 7.15 (s, 2H), 7.16 (d, J = 8.0 Hz, 2H), 7.11-7.07 (m, 1H), 4.57 (dd, J = 4.5, 6.9 Hz, 1H), 4.25 (s, 2H), 3.95 (s, 3H), 3.48 (dd, J = 7.1, 10.5 Hz, 1H), 3.38 (br s, 1H), 3.21 (s, 3H), 2.06 (s, 3H), 1.40-1.31 (m, 4H). LCMS (ESI): m/z = 538.3 [M + H]⁺. |
| | rel-(R)-4-(2-methoxy-1- | | |
| | (oxazol-4- | | |
| | ylmethoxy)ethyl)-2-methyl- | | |
| | N-(1-(2-(1-methyl-1H- | | |
| | pyrazol-4-yl)quinolin-4- | | |
| | yl)cyclopropyl)benzamide, | | |
| | ENT-1 | | |
| Ex. 339 | 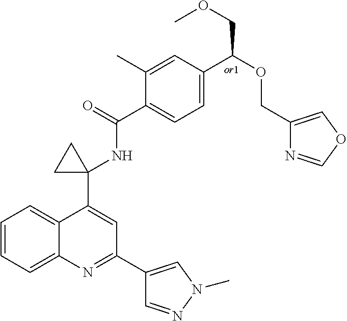 [SMILES: COC[C@@H](OCc1cocn1)c1ccc(C(=O)NC2(c3cc(-c4cnn(C)c4)nc4ccccc34)CC2)c(C)c1] | Same as Ex. 342 | ¹H NMR (400 MHz, DMSO-d6) δ = 9.25 (s, 1H), 8.57 (d, J = 8.2 Hz, 1H), 8.54 (s, 1H), 8.32 (s, 1H), 8.18 (s, 1H), 8.03 (s, 1H), 7.98- 7.93 (m, 2H), 7.71 (t, J = 7.2 Hz, 1H), 7.58-7.52 (m, 1H), 7.16 (d, J = 8.0 Hz, 2H), 7.15 (s, 1H), 7.11- 7.07 (m, 1H), 4.57 (dd, J = 4.4, 6.8 Hz, 1H), 4.25 (s, 2H), 3.95 (s, 3H), 3.48 (dd, J = 7.0, 10.5 Hz, 1H), 3.40-3.38 (m, 1H), 3.21 (s, 3H), 2.06 (s, 3H), 1.40-1.31 (m, 4H). LCMS (ESI): m/z = 538.3 [M + H]⁺. |
| | rel-(R)-4-(2-methoxy-1- | | |
| | (oxazol-4-ylmethoxy)ethyl)- | | |
| | 2-methyl-N-(1-(2-(1- | | |
| | methyl-1H-pyrazol-4- | | |
| | yl)quinolin-4-yl)cyclo- | | |
| | propyl)benzamide, ENT-2 | | |
| Ex. 340 | 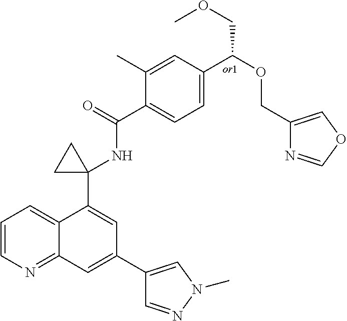 [SMILES: COC[C@H](OCc1cocn1)c1ccc(C(=O)NC2(c3cc(-c4cnn(C)c4)cc4ncccc34)CC2)c(C)c1] | Example 324, step 5-8 using 4- (chloromethyl) oxazole instead of 4- (chloromethyl) thiazole in step 5 and 1-(7-(1-methyl-1H- pyrazol-4- yl)quinolin-5- yl)cyclopropan-1- amine instead of 1- (2-(1-methyl-1H- pyrazol-4- yl)quinolin-4- yl)cyclopropan-1- amine in step 7 | ¹H NMR (400 MHz, DMSO-d6) δ = 9.17 (s, 1H), 8.99 (d, J = 8.5 Hz, 1H), 8.88 (dd, J = 1.5, 4.1 Hz, 1H), 8.41 (s, 1H), 8.32 (s, 1H), 8.12- 8.02 (m, 4H), 7.51 (dd, J = 4.1, 8.5 Hz, 1H), 7.15 (d, J = 5.7 Hz, 2H), 7.14 (s, 1H), 7.10-7.04 (m, 1H), 4.57 (dd, J = 4.4, 6.9 Hz, 1H), 4.24 (s, 2H), 3.93 (s, 3H), 3.48 (dd, J = 7.1, 10.5 Hz, 1H), 3.42-3.35 (m, 1H), 3.21 (s, 3H), 2.06 (s, 3H), 1.37 (br s, 2H), 1.33-1.26 (m, 2H). LCMS (ESI): m/z = 538.3 [M + H]⁺. |
| | rel-(R)-4-(2-methoxy-1- | | |
| | (oxazol-4- | | |
| | ylmethoxy)ethyl)-2-methyl- | | |
| | N-(1-(7-(1-methyl-1H- | | |
| | pyrazol-4-yl)quinolin-5- | | |
| | yl)cyclopropyl)benzamide, | | |
| | ENT-1 | | |
| Ex. 341 | 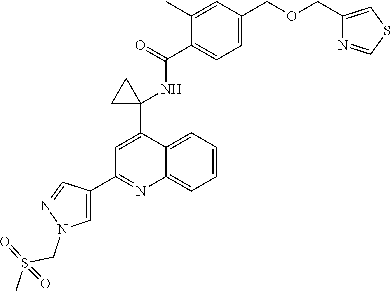 [SMILES: Cc1cc(COCc2cscn2)ccc1C(=O)NC1(c2cc(-c3cnn(CS(C)(=O)=O)c3)nc3ccccc23)CC1] | Example 328, step 2 using N-(1-(2- chloroquinolin-4- yl)cyclopropyl)-2- methyl-4-((thiazol- 4-ylmethoxy) methyl)benzamide and 1- ((methylsulfonyl) methyl)-4-(4,4,5,5- tetramethyl-1,3,2- dioxaborolan-2-yl)- 1H-pyrazole | ¹H NMR (400 MHz, DMSO-d6) δ = 9.25 (s, 1H), 9.07 (d, J = 2.0 Hz, 1H), 8.69 (s, 1H), 8.59 (d, J = 8.4 Hz, 1H), 8.41 (s, 1H), 8.03-7.97 (m, 2H), 7.73 (t, J = 7.2 Hz, 1H), 7.65-7.61 (m, 1H), 7.58 (dt, J = 1.2, 7.6 Hz, 1H), 7.12 (s, 2H), 7.13 (d, J = 6.6 Hz, 2H), 7.10-7.06 (m, 1H), 5.84 (s, 2H), 4.59 (s, 2H), 4.50 (s, 2H), 3.12-3.06 (m, 3H), 2.50 (br s, 3H), 2.05 (s, 3H), 1.37 (br s, 4H). LCMS (ESI): m/z = 588.3 [M + H⁺. |
| | 2-methyl-N-(1-(2-(1- | | |
| | ((methylsulfonyl)methyl)- | | |
| | 1H-pyrazol-4-yl)quinolin-4- | | |
| | yl)cyclopropyl)-4-((thiazol- | | |
| | 4-ylmethoxy)methyl) | | |
| | benzamide | | |
| Ex. 343 | 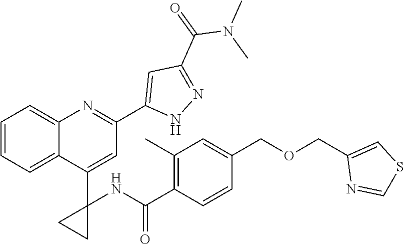 [SMILES: Cc1cc(COCc2cscn2)ccc1C(=O)NC1(c2cc(-c3cc(C(=O)N(C)C)n[nH]3)nc3ccccc23)CC1] | Example 274, step 6 using 2-methyl-4- ((thiazol-4- ylmethoxy)methyl) benzoic acid and 5- (4-(1- aminocyclopropyl) quinolin-2-yl)-N,N- dimethyl-1H- pyrazole-3- carboxamide | ¹H NMR (400 MHz, DMSO-d6) δ = 14.27-13.75 (m, 1H), 9.26 (br d, J = 8.5 Hz, 1H), 9.07 (d, J = 1.9 Hz, 1H), 8.64 (d, J = 8.4 Hz, 1H), 8.49- 8.14 (m, 1H), 8.13-8.00 (m, 1H), 7.86-7.72 (m, 1H), 7.71-7.57 (m, 2H), 7.44-7.26 (m, 1H), 7.18- 7.05 (m, 3H), 4.60 (s, 2H), 4.51 (s, 2H), 3.30 (s, 3H), 3.04 (br d, J = 8.6 Hz, 3H), 2.04 (s, 3H), 1.48- 1.22 (m, 4H). LCMS (ESI): m/z = 567.3 [M + H]⁺. |
| | N,N-dimethyl-5-(4-(1-(2- | | |
| | methyl-4-((thiazol-4- | | |
| | ylmethoxy)methyl)benzamido) | | |
| | cyclopropyl)quinolin-2- | | |
| | yl)-1H-pyrazole-3- | | |
| | carboxamide | | |
| Ex. 344 | 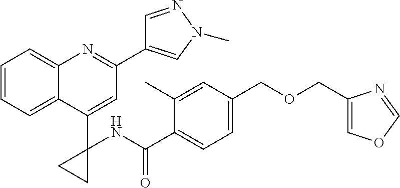 [SMILES: Cc1cc(COCc2cocn2)ccc1C(=O)NC1(c2cc(-c3cnn(C)c3)nc3ccccc23)CC1] | Example 274, step 6 using 2-methyl-4- ((oxazol-4- ylmethoxy)methyl) benzoic acid and 1- (2-(1-methyl-1H- pyrazol-4- yl)quinolin-4- yl)cyclopropan-1- amine | ¹H NMR (400 MHz, MeOD-d4) δ = 8.54 (d, J = 8.0 Hz, 1H), 8.39 (s, 1H), 8.25 (s, 1H), 8.18 (s, 1H), 8.09-8.03 (m, 2H), 7.89 (s, 1H), 7.77-7.71 (m, 1H), 7.62-7.57 (m, 1H), 7.16-7.12 (m, 2H), 7.11- 7.07 (m, 1H), 4.51 (s, 2H), 4.45 (s, 2H), 4.01 (s, 3H), 2.09 (s, 3H), 1.53-1.45 (m, 2H), 1.45-1.38 (m, 2H). LCMS (ESI): m/z = 494.3 [M + H]⁺. |
| | 2-methyl-N-(1-(2-(1- | | |
| | methyl-1H-pyrazol-4- | | |
| | yl)quinolin-4- | | |
| | yl)cyclopropyl)-4-((oxazol- | | |
| | 4-ylmethoxy)methyl) | | |
| | benzamide | | |
| Ex. 345 | 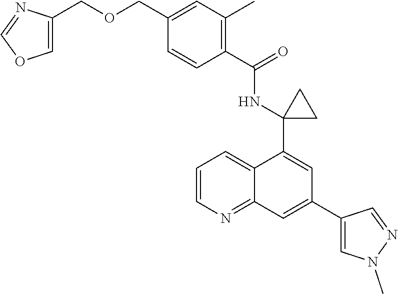 [SMILES: Cc1cc(COCc2cocn2)ccc1C(=O)NC1(c2cc(-c3cnn(C)c3)cc3ncccc23)CC1] | Example 274, step 6 using 2-methyl-4- ((oxazol-4- ylmethoxy)methyl) benzoic acid and 1- (7-(1-methyl-1H- pyrazol-4- yl)quinolin-5- yl)cyclopropan-1- amine | ¹H NMR (400 MHz, MeOD-d4) δ = 9.09-9.03 (m, 1H), 8.84 (dd, J = 1.6, 4.3 Hz, 1H), 8.25-8.22 (m, 2H), 8.18 (s, 1H), 8.10-8.06 (m, 2H), 7.89 (d, J = 0.9 Hz, 1H), 7.54 (dd, J = 4.3, 8.5 Hz, 1H), 7.16- 7.12 (m, 2H), 7.11-7.06 (m, 1H), 4.50 (s, 2H), 4.44 (s, 2H), 3.99 (s, 3H), 2.10 (s, 3H), 1.52-1.46 (m, 2H), 1.41-1.36 (m, 2H). LCMS (ESI): m/z = 494.4 [M + H]⁺. |
| | 2-methyl-N-(1-(7-(1- | | |
| | methyl-1H-pyrazol-4- | | |
| | yl)quinolin-5- | | |
| | yl)cyclopropyl)-4-((oxazol- | | |
| | 4-ylmethoxy)methyl) | | |
| | benzamide | | |
| Ex. 346 | 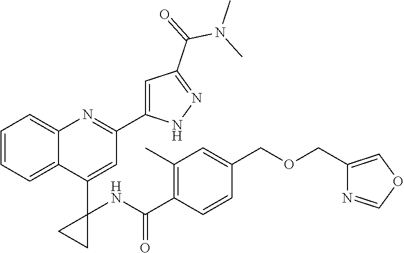 [SMILES: Cc1cc(COCc2cocn2)ccc1C(=O)NC1(c2cc(-c3cc(C(=O)N(C)C)n[nH]3)nc3ccccc23)CC1] | Example 274, step 6 using 2-methyl-4- ((oxazol-4- ylmethoxy)methyl) benzoic acid and 5- (4-(1- aminocyclopropyl) quinolin-2-yl)-N,N- dimethyl-1H- pyrazole-3- carboxamide | ¹H NMR (400 MHz, MeOD-d4) δ = 8.60 (d, J = 8.2 Hz, 1H), 8.37- 8.12 (m, 3H), 7.89 (d, J = 0.7 Hz, 1H), 7.79 (dt, J = 1.2, 7.7 Hz, 1H), 7.69-7.62 (m, 1H), 7.41 (s, 1H), 7.14 (d, J = 4.9 Hz, 2H), 7.12- 7.07 (m, 1H), 4.50 (s, 2H), 4.44 (s, 2H), 3.40 (s, 3H), 3.15 (s, 3H), 2.08 (s, 3H), 1.53-1.48 (m, 2H), 1.47-1.42 (m, 2H). LCMS (ESI): m/z = 551.3 [M + H]⁺. |
| | N,N-dimethyl-5-(4-(1-(2- | | |
| | methyl-4-((oxazol-4- | | |
| | ylmethoxy)methyl)benzamido) | | |
| | cyclopropyl)quinolin-2- | | |
| | yl)-1H-pyrazole-3- | | |
| | carboxamide | | |
| Ex. 347 | 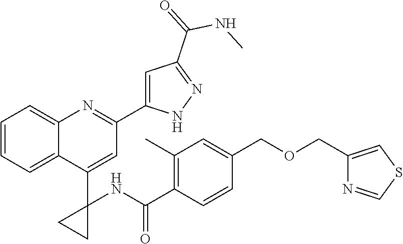 [SMILES: CNC(=O)c1cc(-c2cc(C3(NC(=O)c4ccc(COCc5cscn5)cc4C)CC3)c3ccccc3n2)[nH]n1] | Example 274, step 6 using 2-methyl-4- ((thiazol-4- ylmethoxy)methyl) benzoic acid and 3- (4-(1- aminocyclopropyl) quinolin-2-yl)-N- methyl-1H- pyrazole-5- carboxamide | ¹H NMR (400 MHz, METHANOL- d4) δ = 8.98 (d, J = 1.9 Hz, 1H), 8.60 (d, J = 8.3 Hz, 1H), 8.28- 8.17 (m, 1H), 8.15 (br d, J = 8.5 Hz, 1H), 7.79 (t, J = 7.2 Hz, 1H), 7.67 (br d, J = 7.4 Hz, 1H), 7.54- 7.44 (m, 2H), 7.36-7.25 (m, 1H), 7.18-7.12 (m, 2H), 7.12-7.09 (m, 1H), 4.65 (s, 2H), 4.54 (s, 2H), 2.95 (s, 3H), 2.08 (s, 3H), 1.53- 1.49 (m, 2H), 1.47-1.42 (m, 2H). LCMS (ESI): m/z = 553.3 [M + H]⁺. |
| | N-methyl-5-(4-(1-(2- | | |
| | methyl-4-((thiazol-4- | | |
| | ylmethoxy)methyl)benzamido) | | |
| | cyclopropyl)quinolin-2- | | |
| | yl)-1H-pyrazole-3- | | |
| | carboxamide | | |
| Ex. 348 | 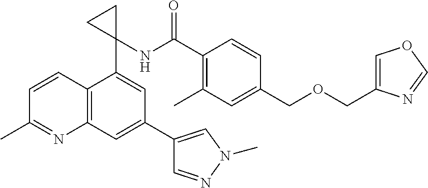 [SMILES: Cc1ccc2c(C3(NC(=O)c4ccc(COCc5cocn5)cc4C)CC3)cc(-c3cnn(C)c3)cc2n1] | Example 274, step 6 using 2-methyl-4- ((oxazol-4- ylmethoxy)methyl) benzoic acid and 1- (2-methyl-7-(1- methyl-1H-pyrazol- 4-yl)quinolin-5- yl)cyclopropan-1- amine | ¹H NMR (400 MHz, MeOD-d4) δ = 8.93 (d, J = 8.8 Hz, 1H), 8.20 (d, J = 10.0 Hz, 2H), 8.15 (d, J = 1.7 Hz, 1H), 8.05 (d, J = 0.6 Hz, 1H), 8.00 (d, J = 0.9 Hz, 1H), 7.89 (d, J = 0.9 Hz, 1H), 7.43 (d, J = 8.7 Hz, 1H), 7.16-7.12 (m, 2H), 7.11-7.06 (m, 1H), 4.50 (s, 2H), 4.44 (s, 2H), 3.98 (s, 3H), 2.73 (s, 3H), 2.12 (s, 3H), 1.51-1.44 (m, 2H), 1.39- 1.33 (m, 2H). LCMS (ESI): m/z = 508.4 [M + H]⁺. |
| | 2-methyl-N-(1-(2-methyl-7- | | |
| | (1-methyl-1H-pyrazol-4- | | |
| | yl)quinolin-5- | | |
| | yl)cyclopropyl)-4-((oxazol- | | |
| | 4-ylmethoxy)methyl) | | |
| | benzamide | | |
| Ex. 349 | 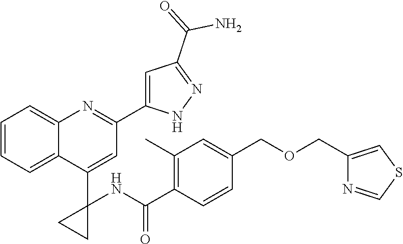 [SMILES: Cc1cc(COCc2cscn2)ccc1C(=O)NC1(c2cc(-c3cc(C(N)=O)n[nH]3)nc3ccccc23)CC1] | Example 274, step 6 using 2-methyl-4- ((thiazol-4-yl- methoxy)methyl) benzoic acid and 3- (4-(1-amino- cyclopropyl) quinolin-2-yl)-1H- pyrazole-5- carboxamide | ¹H NMR (400 MHz, DMSO-d6) δ = 14.37-13.52 (m, 1H), 9.25 (br s, 1H), 9.07 (d, J = 1.9 Hz, 1H), 8.64 (d, J = 8.5 Hz, 1H), 8.52-7.90 (m, 3H), 7.77 (br s, 1H), 7.69-7.44 (m, 4H), 7.17-7.05 (m, 3H), 4.60 (s, 2H), 4.51 (s, 2H), 2.05 (s, 3H), 1.40 (br s, 4H). LCMS (ESI): m/z = 539.3 [M + H]⁺. |
| | 5-(4-(1-(2-methyl-4- | | |
| | ((thiazol-4-ylmethoxy) | | |
| | methyl)benzamido)cyclo- | | |
| | propyl)quinolin-2-yl)-1H- | | |
| | pyrazole-3-carboxamide | | |
| Ex. 350 | 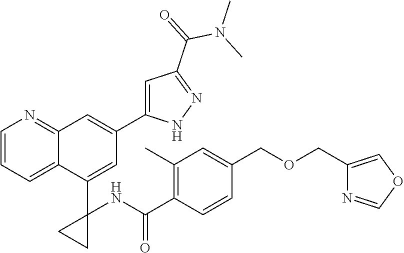 [SMILES: Cc1cc(COCc2cocn2)ccc1C(=O)NC1(c2cc(-c3cc(C(=O)N(C)C)n[nH]3)cc3ncccc23)CC1] | Example 274, step 6 using 2-methyl-4- ((oxazol-4- ylmethoxy)methyl) benzoic acid and 3- (5-(1- aminocyclopropyl) quinolin-7-yl)-N,N- dimethyl-1H- pyrazole-5- carboxamide | ¹H NMR (400 MHz, DMSO-d6) δ = 13.96-13.66 (m, 1H), 9.17 (br s, 1H), 9.09-9.02 (m, 1H), 8.99- 8.88 (m, 1H), 8.49-8.17 (m, 3H), 8.08 (s, 1H), 7.59 (br s, 1H), 7.43- 7.19 (m, 1H), 7.16-7.02 (m, 3H), 4.46 (s, 2H), 4.37 (s, 2H), 3.04 (br d, J = 9.0 Hz, 3H), 2.52-2.51 (m, 3H), 2.09-2.00 (m, 3H), 1.44- 1.21 (m, 4H). LCMS (ESI): m/z = 551.4 [M + H]⁺. |
| | N,N-dimethyl-5-(5-(1-(2- | | |
| | methyl-4-((oxazol-4- | | |
| | ylmethoxy)methyl) | | |
| | benzamido)cyclopropyl) | | |
| | quinolin-7-yl)-1H-pyrazole- | | |
| | 3-carboxamide | | |
| Ex. 351 | 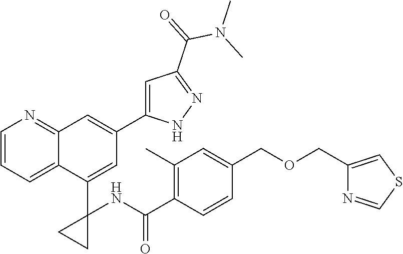 [SMILES: Cc1cc(COCc2cscn2)ccc1C(=O)NC1(c2cc(-c3cc(C(=O)N(C)C)n[nH]3)cc3ncccc23)CC1] | Example 274, step 6 using 2-methyl-4- ((thiazol-4- ylmethoxy)methyl) benzoic acid and 3- (5-(1- aminocyclopropyl) quinolin-7-yl)-N,N- dimethyl-1H- pyrazole-5- carboxamide | ¹H NMR (400 MHz, DMSO-d6) δ = 13.79 (br s, 1H), 9.17 (s, 1H), 9.10- 9.01 (m, 2H), 8.93 (br d, J = 2.9 Hz, 1H), 8.44-8.20 (m, 2H), 7.65- 7.53 (m, 2H), 7.30 (br s, 1H), 7.16- 7.03 (m, 3H), 4.59 (s, 2H), 4.50 (s, 2H), 3.32 (br s, 3H), 3.04 (s, 3H), 2.04 (s, 3H), 1.44-1.24 (m, 4H). LCMS (ESI): m/z = 567.3 [M + H]⁺. |
| | N,N-dimethyl-5-(5-(1-(2- | | |
| | methyl-4-((thiazol-4- | | |
| | ylmethoxy)methyl) | | |
| | benzamido)cyclopropyl) | | |
| | quinolin-7-yl)-1H-pyrazole-3- | | |
| | carboxamide | | |
| Ex. 355 | 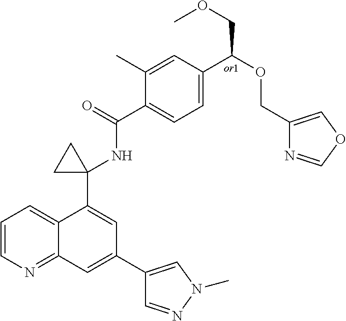 [SMILES: COC[C@@H](OCc1cocn1)c1ccc(C(=O)NC2(c3cc(-c4cnn(C)c4)cc4ncccc34)CC2)c(C)c1] | Example 324, step 5-8 using 4- (chloromethyl) oxazole instead of 4-(chloromethyl) thiazole in step 5 and 1-(7-(1-methyl- 1H-pyrazol-4- yl)quinolin-5- yl)cyclopropan-1- amine instead of 1- (2-(1-methyl-1H- pyrazol-4- yl)quinolin-4- yl)cyclopropan-1- amine in step 7 | ¹H NMR (400 MHz, DMSO-d6) δ = 9.17 (s, 1H), 8.99 (d, J = 8.5 Hz, 1H), 8.88 (dd, J = 1.3, 4.1 Hz, 1H), 8.42 (s, 1H), 8.32 (s, 1H), 8.11- 8.07 (m, 2H), 8.05 (d, J = 10.0 Hz, 2H), 7.51 (dd, J = 4.2, 8.6 Hz, 1H), 7.15 (d, J = 5.2 Hz, 2H), 7.14 (s, 1H), 7.10-7.05 (m, 1H), 4.57 (dd, J = 4.4, 6.9 Hz, 1H), 4.24 (s, 2H), 3.93 (s, 3H), 3.48 (dd, J = 7.0, 10.5 Hz, 1H), 3.39-3.35 (m, 1H), 3.21 (s, 3H), 2.06 (s, 3H), 1.37 (br s, 2H), 1.30 (br s, 2H). LCMS: m/z = 538.3 [M + H]⁺. |
| | rel-(R)-4-(2-methoxy-1- | | |
| | (oxazol-4-ylmethoxy)ethyl)- | | |
| | 2-methyl-N-(1-(7-(1- | | |
| | methyl-1H-pyrazol-4- | | |
| | yl)quinolin-5-yl)cyclo- | | |
| | propyl)benzamide, ENT-1 | | |
| Ex. 356 | 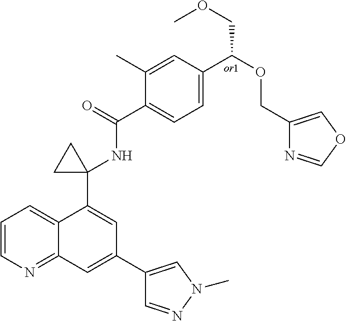 [SMILES: COC[C@H](OCc1cocn1)c1ccc(C(=O)NC2(c3cc(-c4cnn(C)c4)cc4ncccc34)CC2)c(C)c1] | Same as Ex. 355 | ¹H NMR (400 MHz, DMSO-d6) δ = 9.17 (s, 1H), 8.99 (d, J = 8.5 Hz, 1H), 8.88 (dd, J = 1.5, 4.1 Hz, 1H), 8.41 (s, 1H), 8.32 (s, 1H), 8.12- 8.02 (m, 4H), 7.51 (dd, J = 4.1, 8.5 Hz, 1H), 7.15 (d, J = 5.7 Hz, 2H), 7.14 (s, 1H), 7.10-7.04 (m, 1H), 4.57 (dd, J = 4.4, 6.9 Hz, 1H), 4.24 (s, 2H), 3.93 (s, 3H), 3.48 (dd, J = 7.1, 10.5 Hz, 1H), 3.42-3.35 (m, 1H), 3.21 (s, 3H), 2.06 (s, 3H), 1.37 (br s, 2H), 1.33-1.26 (m, 2H). LCMS: m/z = 538.3 [M + H]⁺. |
| | rel-(R)-4-(2-methoxy-1- | | |
| | (oxazol-4-ylmethoxy)ethyl)- | | |
| | 2-methyl-N-(1-(7-(1- | | |
| | methyl-1H-pyrazol-4- | | |
| | yl)quinolin-5-yl)cyclo- | | |
| | propyl)benzamide, ENT-2 | | |
| Ex. 357 | 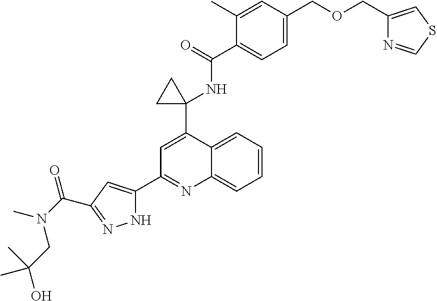 [SMILES: Cc1cc(COCc2cscn2)ccc1C(=O)NC1(c2cc(-c3cc(C(=O)N(C)CC(C)(C)O)n[nH]3)nc3ccccc23)CC1] | Example 329 step 3-4 using 5-(4-(1- (2-methyl-4- ((thiazol-4- ylmethoxy)methyl) benzamido) cyclopropyl) quinolin-2-yl)-1- (tetrahydro-2H- pyran-2-yl)-1H- pyrazole-3- carboxylic acid and 2-methyl-1- (methylamino) propan-2-ol | ¹H NMR (400 MHz, DMSO-d6) δ = 14.12-13.72 (m, 1H), 9.27 (s, 1H), 9.08 (d, J = 2.0 Hz, 1H), 8.65 (br d, J = 8.3 Hz, 1H), 8.46-8.16 (m, 1H), 8.08 (br d, J = 8.4 Hz, 1H), 7.82-7.74 (m, 1H), 7.68-7.59 (m, 2H), 7.38 (br s, 1H), 7.14 (s, 1H), 7.15 (d, J = 8.0 Hz, 1H), 7.12- 7.08 (m, 1H), 4.66-4.63 (m, 1H), 4.62-4.59 (m, 2H), 4.52 (s, 2H), 3.52 (s, 1H), 3.41 (s, 2H), 3.32- 3.22 (m, 1H), 3.13 (s, 1H), 2.05 (s, 3H), 1.49-1.32 (m, 4H), , 1.16 (s, 4H), 1.04 (s, 2H). LCMS (ESI): m/z = 625.3 [M + H]⁺. |
| | N-(2-hydroxy-2- | | |
| | methylpropyl)-N-methyl-5- | | |
| | (4-(1-(2-methyl-4-((thiazol- | | |
| | 4-ylmethoxy)methyl) | | |
| | benzamido)cyclopropyl) | | |
| | quinolin-2-yl)-1H-pyrazole- | | |
| | 3-carboxamide | | |
| Ex. 359 | 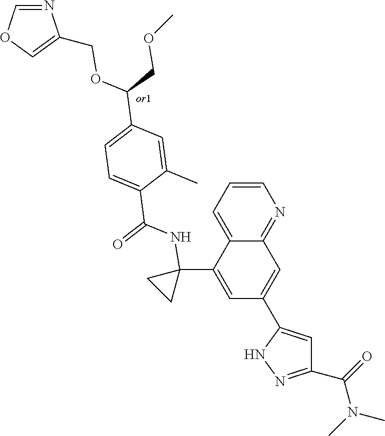 [SMILES: COC[C@H](OCc1cocn1)c1ccc(C(=O)NC2(c3cc(-c4cc(C(=O)N(C)C)n[nH]4)cc4ncccc34)CC2)c(C)c1] | Example 324, step 5-8 using 4- (chloromethyl) oxazole instead of 4-(chloromethyl) thiazole in step 5 and 3-(5-(1- aminocyclopropyl) quinolin-7-yl)-N,N- dimethyl-1H- pyrazole-5- carboxamide instead of 1-(2-(1- methyl-1H-pyrazol- 4-yl)quinolin-4- yl)cyclopropan-1- amine in step 7 | 1H NMR (400 MHz, METHANOL- d4) δ = 9.13 (br d, J = 8.4 Hz, 1H), 8.93 (br s, 1H), 8.60-8.27 (m, 2H), 8.15 (s, 1H), 7.86 (s, 1H), 7.64 (br s, 1H), 7.18 (s, 3H), 7.16- 7.11 (m, 1H), 4.60-4.55 (m, 2H), 4.39-4.31 (m, 2H), 3.57 (dd, J = 7.3, 10.5 Hz, 1H), 3.42-3.38 (m, 3H), 3.32-3.29 (m, 3H), 3.23- 3.13 (m, 3H), 2.15-2.08 (m, 3H), 1.58-1.41 (m, 4H). LCMS (ESI): m/z = 595.3 [M + H]+. |
| | rel-(R)-5-(5-(1-(4-(2- | | |
| | methoxy-1-(oxazol-4- | | |
| | ylmethoxy)ethyl)-2- | | |
| | methylbenzamido)cyclo- | | |
| | propyl)quinolin-7-yl)-N,N- | | |
| | dimethyl-1H-pyrazole-3- | | |
| | carboxamide, ENT-1 | | |
| Ex. 360 | 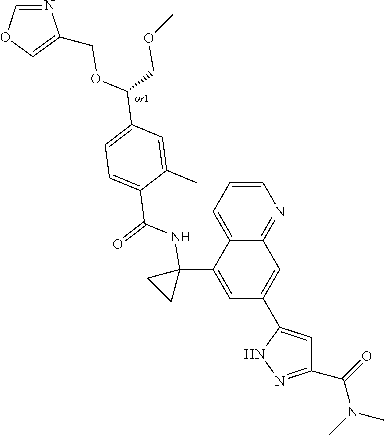 [SMILES: COC[C@@H](OCc1cocn1)c1ccc(C(=O)NC2(c3cc(-c4cc(C(=O)N(C)C)n[nH]4)cc4ncccc34)CC2)c(C)c1] | Same as Ex. 359 | ¹H NMR (400 MHz, METHANOL- d4) δ = 9.13 (br d, J = 8.3 Hz, 1H), 8.93 (br s, 1H), 8.58-8.28 (m, 2H), 8.15 (s, 1H), 7.86 (s, 1H), 7.65 (br s, 1H), 7.34-7.17 (m, 3H), 7.15-7.11 (m, 1H), 4.58 (br s, 2H), 4.42-4.31 (m, 2H), 3.57 (dd, J = 7.4, 10.6 Hz, 1H), 3.42- 3.38 (m, 3H), 3.31 (s, 3H), 3.22- 3.13 (m, 3H), 2.12 (s, 3H), 1.59- 1.41 (m, 4H). LCMS (ESI): m/z = 595.3 [M + H]+. |
| | rel-(R)-5-(5-(1-(4-(2- | | |
| | methoxy-1-(oxazol-4- | | |
| | ylmethoxy)ethyl)-2- | | |
| | methylbenzamido)cyclo- | | |
| | propyl)quinolin-7-yl)-N,N- | | |
| | dimethyl-1H-pyrazole-3- | | |
| | carboxamide, ENT-2 | | |
| Ex. 361 | 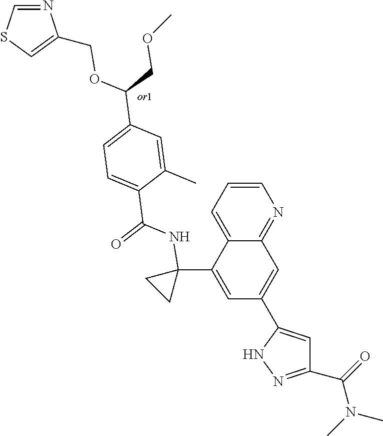 [SMILES: COC[C@H](OCc1cscn1)c1ccc(C(=O)NC2(c3cc(-c4cc(C(=O)N(C)C)n[nH]4)cc4ncccc34)CC2)c(C)c1] | Example 324, step 5-7 using 3-(5-(1- aminocyclopropyl) quinolin-7-yl)-N,N- dimethyl-1H- pyrazole-5- carboxamide instead of 1-(2-(1- methyl-1H-pyrazol- 4-yl)quinolin-4- yl)cyclopropan-1- amine in step 7 | ¹H NMR (400 MHz, DMSO-d6) δ = 13.97-13.65 (m, 1H), 9.19 (br s, 1H), 9.08-9.01 (m, 2H), 8.93 (br d, J = 6.1 Hz, 1H), 8.47-8.20 (m, 2H), 7.64-7.51 (m, 2H), 7.41- 7.20 (m, 1H), 7.18-7.12 (m, 2H), 7.11-7.04 (m, 1H), 4.60 (br d, J = 11.1 Hz, 1H), 4.46 (s, 2H), 3.61- 3.43 (m, 2H), 3.33 (br s, 3H), 3.21 (s, 3H), 3.03 (br s, 3H), 2.04 (s, 3H), 1.42-1.19 (m, 4H). LCMS (ESI): m/z = 611.3 [M + H]+. |
| | rel-(R)-5-(5-(1-(4-(2- | | |
| | methoxy-1-(thiazol-4- | | |
| | ylmethoxy)ethyl)-2- | | |
| | methylbenzamido) | | |
| | cyclopropyl)quinolin-7-yl)- | | |
| | N,N-dimethyl-1H-pyrazole- | | |
| | 3-carboxamide, ENT-1 | | |
| Ex. 362 | 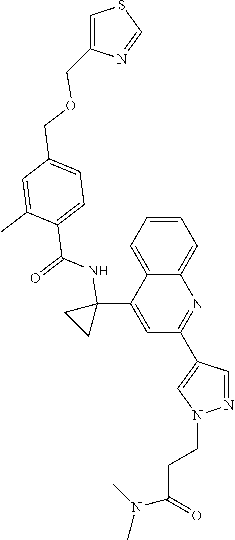 [SMILES: Cc1cc(COCc2cscn2)ccc1C(=O)NC1(c2cc(-c3cnn(CCC(=O)N(C)C)c3)nc3ccccc23)CC1] | Using procedure analogous to the ones described in Example 342 | ¹H NMR (400 MHz, METHANOL- d4) δ = 8.99 (d, J = 2.0 Hz, 1H), 8.56 (d, J = 8.1 Hz, 1H), 8.47 (s, 1H), 8.29 (s, 1H), 8.10 (s, 1H), 8.07 (d, J = 8.4 Hz, 1H), 7.76 (t, J = 7.3 Hz, 1H), 7.61 (t, J = 7.3 Hz, 1H), 7.53 (d, J = 2.0 Hz, 1H), 7.17 (br d, J = 4.2 Hz, 2H), 7.14-7.10 (m, 1H), 4.66 (s, 2H), 4.58-4.54 (m, 4H), 3.06 (t, J = 6.7 Hz, 2H), 3.03 (s, 3H), 2.95 (s, 3H), 2.11 (s, 3H), 1.53-1.41 (m, 4H). LCMS (ESI): m/z = 595.3 [M + H]+. |
| | N,N-dimethyl3-(4-{4-[1-(4- | | |
| | {[(1,3-thiazol-4- | | |
| | yl)methoxy]methyl}-2- | | |
| | toluoylamino)cyclopropyl]-2- | | |
| | quinolyl}-1- | | |
| | pyrazolyl)propionamide | | |
| Ex. 363 | 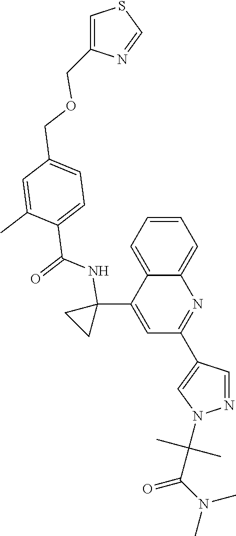 | Using procedure analogous to the ones described in Example 342 | ¹H NMR (400 MHz, METHANOL- d4) δ = 8.98 (d, J = 2.0 Hz, 1H), 8.65 (s, 1H), 8.54 (d, J = 8.1 Hz, 1H), 8.36 (s, 1H), 8.15 (s, 1H), 8.07 (d, J = 8.4 Hz, 1H), 7.75 (t, J = 7.0 Hz, 1H), 7.61 (t, J = 7.3 Hz, 1H), 7.52 (s, 1H), 7.19-7.13 (m, 2H), 7.13-7.09 (m, 1H), 4.65 (s, 2H), 4.54 (s, 2H), 2.98 (br s, 3H), 2.52 (br s, 3H), 2.10 (s, 3H), 1.89 (s, 6H), 1.52-1.41 (m, 4H). LCMS (ESI): m/z = 609.2 [M + H]+. |
| | N,N-dimethyl2-methyl-2-(4- | | |
| | {4-[1-(4-{[(1,3-thiazol-4- | | |
| | yl)methoxy]methyl}-2- | | |
| | toluoylamino)cyclopropyl]-2- | | |
| | quinolyl}-1- | | |
| | pyrazolyl)propionamide | | |
| Ex. 364 | 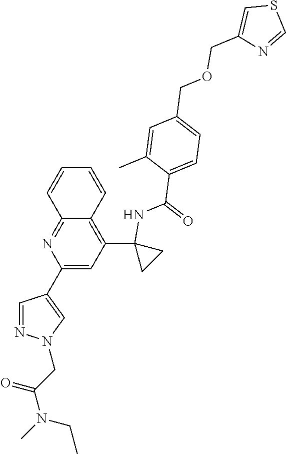 [SMILES: CCN(C)C(=O)Cn1cc(-c2cc(C3(NC(=O)c4ccc(COCc5cscn5)cc4C)CC3)c3ccccc3n2)cn1] | Using procedure analogous to the ones described in Example 342 | ¹H NMR (400 MHz, DMSO-d6) δ = 9.24 (s, 1H), 9.08 (d, J = 2.0 Hz, 1H), 8.58 (d, J = 8.1 Hz, 1H), 8.54- 8.47 (m, 1H), 8.18 (s, 1H), 7.99- 7.95 (m, 2H), 7.71 (t, J = 7.4 Hz, 1H), 7.64 (s, 1H), 7.56 (t, J = 7.5 Hz, 1H), 7.16-7.12 (m, 2H), 7.11- 7.08 (m, 1H), 5.21 (d, J = 6.6 Hz, 2H), 4.61 (s, 2H), 4.52 (s, 2H), 3.49-3.36 (m, 4H), 3.05 (s, 2H), 2.86 (s, 1H), 2.06 (s, 3H), 1.37 (br d, J = 4.4 Hz, 4H), 1.19 (t, J = 7.0 Hz, 2H), 1.05 (t, J = 7.2 Hz, 2H) LCMS (ESI): m/z = 595.3 [M + H]⁺, |
| | N-ethyl-N-methyl(4-{4-[1-(4- | | |
| | {[(1,3-thiazol-4-yl)methoxy] | | |
| | methyl}-2-toluoylamino) | | |
| | cyclopropyl]-2-quinolyl}-1- | | |
| | pyrazolyl)acetamide | | |
| Ex. 365 | 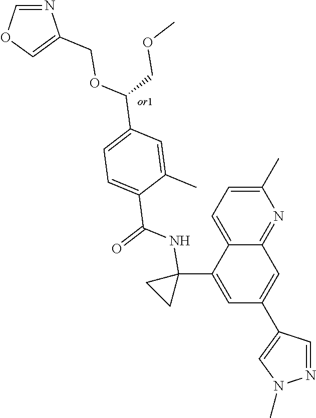 [SMILES: COC[C@@H](OCc1cocn1)c1ccc(C(=O)NC2(c3cc(-c4cnn(C)c4)cc4nc(C)ccc34)CC2)c(C)c1] | Using procedure analogous to the ones described in Example 353 | ¹H NMR (400 MHz, MeOD-d4) δ = 8.96 (d, J = 8.6 Hz, 1H), 8.22 (s, 1H), 8.19-8.12 (m, 2H), 8.06- 8.01 (m, 2H), 7.84 (d, J = 0.9 Hz, 1H), 7.46 (d, J = 8.7 Hz, 1H), 7.17 (d, J = 2.7 Hz, 2H), 7.13-7.09 (m, 1H), 4.58 (dd, J = 4.0, 7.3 Hz, 1H), 4.38-4.28 (m, 2H), 3.99-3.98 (m, 3H), 3.55-3.52 (m, 2H), 3.30 (s, 3H), 2.75 (s, 3H), 2.11 (s, 3H), 1.38 (d, J = 2.9 Hz, 4H). LCMS (ESI): m/z = 552.4 [M + H]+. |
| | rel-(R)-4-(2-methoxy-1- | | |
| | (oxazol-4-ylmethoxy)ethyl)- | | |
| | 2-methyl-N-(1-(2-methyl-7- | | |
| | (1-methyl-1H-pyrazol-4- | | |
| | yl)quinolin-5-yl)cyclopropyl) | | |
| | benzamide, ENT-1 | | |

### Biochemical Data

The compounds of the invention were tested in a Papain Like Protease (PLpro) biochemical assay in order to determine their ability to inhibit the enzyme's activity. Biochemical potencies are reported in Table 1 as the concentration of the compound required to achieve 50% inhibition of the enzyme activity in the assay (IC₅₀).

The potency of compounds against the SARS-CoV-2 Papain like protease (PLpro) was measured using a synthetic profluorogenic substrate Z-RLRGG-AMC. Compounds were serially diluted over 11 points using 100% DMSO as a diluent, from either a top concentration of 3 mM with a half-log dilution factor or a top dose of 0.1 mM with a 2-fold dilution factor. The top dose of compound in the assay was either 30 μM or 1 μM. The assay buffer contained 50 mM HEPES, pH 7.5, 1 mM TCEP and 0.1% BSA; final assay conditions included 1% DMSO, 6.25 nM PLPro enzyme, 25 μM peptide substrate and a 60 minute enzyme reaction time.

Briefly, 250 nL of serially diluted compound was spotted into a white 384-well plate, followed by addition of SARS CoV2 PLpro enzyme (7.8 nM, 10 μL) in assay buffer. The compounds and PLpro enzyme were incubated for 30 minutes at room temperature. The reaction was then initiated by the addition of profluorogenic peptide substrate in assay buffer (5 μL, 125 μM Z-RLRGG-AMC). The reaction was allowed to progress for 60 minutes at 25° C. after which the plate was read on a Molecular Devices Spectramax M2e reader at an Ex/Em of 360 nm/460 nm. The no compound, zero percent inhibition (ZPE) control wells contained 1% DMSO vehicle with substrate and PLpro enzyme. The hundred percent effect (HPE) wells contained an internal Pfizer control compound at a dose sufficient to accomplish complete inhibition, 1% DMSO vehicle, substrate and PLpro enzyme. Data were analyzed with ActivityBase software (ID Business Solutions, Ltd). The raw data were transformed to percent activity values using the average from the ZPE and HPE control wells. The resulting data were fit with the four-parameter logistic fit model to determine the IC₅₀ value. For compounds eliciting high potencies, the percent activity values can be fit to the Morrison equation to derive K, values with the following fixed parameters: enzyme concentration, 6.25 nM; substrate Km, 962 μM; substrate concentration, 25 μM. To qualify inter-experimental performance, the internal control (R)-5-(aminomethyl)-2-methyl-N-(1-(naphthalen-1-yl)ethyl)benzamide (compound 2 from J. Med. Chem. 2009, 52, 16, 5228-5240) was tested in each run.

TABLE 1

| Example # | IC₅₀ (μM) |
|---|---|
| 1 | 1.789 |
| 2 | 1.368 |
| 3 | 0.048 |
| 4 | 0.023 |
| 5 | 0.087 |
| 6 | 0.019 |
| 7 | 0.013 |
| 8 | 0.025 |
| 9 | 3.919 |
| 10 | 0.033 |
| 11 | 0.027 |
| 12 | 0.532 |
| 13 | 0.024 |
| 14 | 0.019 |
| 15 | 0.495 |
| 16 | 0.149 |
| 17 | 0.022 |
| 18 | 0.027 |
| 19 | 0.015 |
| 20 | 0.041 |
| 21 | 0.016 |
| 22 | 0.017 |
| 23 | 0.293 |
| 24 | 0.25 |
| 25 | 0.1 |
| 26 | n/a |
| 27 | 0.057 |
| 28 | 5.452 |
| 29 | 0.1 |
| 30 | 7.728 |
| 31 | 0.674 |
| 32 | 0.676 |
| 33 | 1.363 |
| 34 | 1.454 |
| 35 | 1.479 |
| 36 | 0.21 |
| 37 | 10.014 |
| 38 | 1.631 |
| 39 | 0.861 |
| 40 | 3.159 |
| 41 | 1.011 |
| 42 | 2.166 |
| 43 | 3.085 |
| 44 | 3.968 |
| 45 | 3.483 |
| 46 | 3.621 |
| 47 | 3.135 |
| 48 | 0.28 |
| 49 | 0.151 |
| 50 | 0.489 |
| 51 | 0.019 |
| 52 | 0.011 |
| 53 | 0.478 |
| 54 | 0.028 |
| 55 | 0.018 |
| 56 | 0.02 |
| 57 | 0.013 |
| 58 | 100.0 |
| 59 | 0.025 |
| 60 | 0.012 |
| 61 | 0.046 |
| 62 | 0.039 |
| 63 | 0.003 |
| 64 | 0.005 |
| 65 | 0.021 |
| 66 | 0.008 |
| 67 | 0.272 |
| 68 | 0.009 |
| 69 | 0.908 |
| 70 | 0.011 |
| 71 | 0.016 |
| 72 | 0.013 |
| 73 | 0.022 |
| 74 | 0.008 |
| 75 | 0.012 |
| 101 | 0.198 |
| 102 | 0.904 |
| 103 | 0.616 |
| 104 | 10.014 |
| 105 | 0.266 |
| 106 | 0.241 |
| 107 | 0.757 |
| 108 | 0.931 |
| 109 | 0.28 |
| 110 | 0.224 |
| 111 | 17.298 |
| 112 | 0.294 |
| 113 | 0.79 |
| 114 | 0.877 |
| 115 | 1.08 |
| 116 | 0.406 |
| 117 | 0.412 |
| 118 | 1.315 |
| 119 | 0.244 |
| 120 | 0.345 |
| 121 | 1.417 |
| 122 | 0.201 |
| 123 | 0.329 |
| 124 | 0.635 |
| 125 | 0.294 |
| 126 | 0.845 |
| 127 | 1.823 |
| 128 | 0.164 |
| 129 | 0.666 |
| 130 | 3.099 |
| 131 | 2.915 |
| 132 | 0.844 |
| 133 | 0.392 |
| 134 | 0.432 |
| 135 | 0.855 |
| 136 | 1.411 |
| 137 | 1.126 |
| 138 | 0.944 |
| 139 | 0.675 |
| 140 | 1.959 |
| 141 | 0.778 |
| 142 | 0.9 |
| 143 | 8.477 |
| 144 | 1.112 |
| 145 | 2.516 |
| 146 | 4.503 |
| 147 | 0.909 |
| 148 | 1.766 |
| 149 | 2.17 |
| 150 | 1.072 |
| 151 | 1.459 |
| 152 | 0.623 |
| 153 | 0.311 |
| 154 | 0.988 |
| 155 | 0.581 |
| 156 | 7.224 |
| 157 | 2.685 |
| 158 | 5.476 |
| 159 | 1.974 |
| 200 | 0.792 |
| 201 | 0.003 |
| 202 | 0.012 |
| 203 | 0.002 |
| 204 | 0.073 |
| 205 | 0.043 |
| 206 | 0.006 |
| 207 | 0.012 |
| 208 | 0.005 |
| 209 | 0.005 |
| 210 | 0.006 |
| 211 | 0.003 |
| 212 | 0.058 |
| 213 | 0.219 |
| 214 | 0.169 |
| 215 | 0.144 |
| 216 | 0.012 |
| 217 | 0.119 |
| 218 | 0.003 |
| 219 | 0.001 |
| 220 | 0.002 |
| 221 | 0.015 |
| 222 | 1.98 |
| 223 | 0.058 |
| 224 | 0.006 |
| 225 | 0.006 |
| 226 | 0.017 |
| 227 | 0.002 |
| 228 | 0.007 |
| 229 | 0.006 |
| 230 | 0.003 |
| 231 | 0.003 |
| 232 | 0.003 |
| 233 | 0.003 |
| 234 | 0.128 |
| 235 | 0.091 |
| 236 | 0.003 |
| 237 | 0.036 |
| 238 | 0.016 |
| 239 | 0.007 |
| 240 | 0.026 |
| 241 | 0.007 |
| 242 | 0.006 |
| 243 | 0.077 |
| 244 | 0.167 |
| 245 | 0.005 |
| 246 | 0.035 |
| 247 | 0.091 |
| 248 | 0.39 |
| 249 | 0.733 |
| 250 | 0.002 |
| 251 | 0.005 |
| 252 | 0.005 |
| 253 | 0.006 |
| 254 | 0.005 |
| 255 | 0.019 |
| 256 | 0.015 |
| 257 | 0.022 |
| 258 | 0.044 |
| 259 | 0.147 |
| 260 | 0.244 |
| 261 | 0.021 |
| 262 | 0.008 |
| 263 | 0.231 |
| 264 | 0.022 |
| 265 | 0.14 |
| 266 | 0.024 |
| 267 | 0.21 |
| 268 | 0.139 |
| 269 | 0.041 |
| 270 | 0.037 |
| 271 | 0.005 |
| 272 | 0.006 |
| 273 | 0.003 |
| 274 | 0.002 |
| 275 | 0.001 |
| 276 | 0.003 |
| 277 | 0.002 |
| 278 | 0.006 |
| 279 | 0.001 |
| 280 | 0.017 |
| 281 | 0.003 |
| 282 | 0.006 |
| 283 | 0.005 |
| 284 | 0.006 |
| 285 | 0.003 |
| 286 | 0.005 |
| 287 | 0.002 |
| 288 | 0.003 |
| 289 | 0.001 |
| 290 | 0.001 |
| 291 | 0.001 |
| 292 | <0.001 |
| 293 | 0.001 |
| 294 | 0.002 |
| 295 | 0.003 |
| 296 | 0.003 |
| 297 | 0.004 |
| 298 | <0.001 |
| 299 | 0.001 |
| 300 | 0.002 |
| 301 | 0.001 |
| 302 | 0.006 |
| 303 | 0.001 |
| 304 | 0.003 |
| 305 | 0.003 |
| 306 | 0.004 |
| 307 | 0.002 |
| 308 | 0.001 |
| 309 | 0.002 |
| 310 | 0.003 |
| 311 | 0.004 |
| 312 | 0.002 |
| 313 | 0.004 |
| 314 | 0.002 |
| 315 | 0.001 |
| 316 | 0.002 |
| 317 | 0.006 |
| 318 | 0.001 |
| 319 | 0.001 |
| 320 | 0.004 |
| 321 | 0.003 |
| 322 | 0.007 |
| 323 | <0.001 |
| 324 | <0.001 |
| 325 | 0.001 |
| 326 | 0.004 |
| 327 | 0.001 |
| 328 | <0.001 |
| 329 | <0.001 |
| 330 | <0.001 |
| 331 | <0.001 |
| 332 | 0.002 |
| 333 | <0.001 |
| 334 | <0.001 |
| 335 | <0.001 |
| 336 | <0.001 |
| 337 | <0.001 |
| 338 | 0.003 |
| 339 | <0.001 |
| 340 | 0.002 |
| 341 | <0.001 |
| 342 | <0.001 |
| 343 | <0.001 |
| 344 | <0.001 |
| 345 | <0.001 |
| 346 | <0.001 |
| 347 | <0.001 |
| 348 | <0.001 |
| 349 | <0.001 |
| 350 | <0.001 |
| 351 | <0.001 |
| 352 | <0.001 |
| 353 | <0.001 |
| 354 | <0.001 |
| 355 | <0.001 |
| 356 | 0.002 |
| 357 | <0.001 |
| 358 | <0.001 |
| 359 | <0.001 |
| 360 | 0.001 |
| 361 | <0.001 |
| 362 | <0.001 |
| 363 | <0.001 |
| 364 | <0.001 |
| 365 | <0.001 |
| 366 | <0.001 |

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

1. A compound of Formula (I): 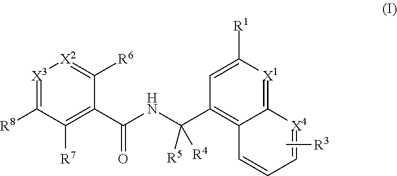
or a pharmaceutically acceptable salt thereof, wherein:
X¹ is N or CR²;
X² is N or CH;
X³ is N or CR⁹ provided that X² and X³ cannot be both N;
X⁴ is CH or N, provided that X¹ and X⁴ are not N at the same time and X⁴ can only be N when R⁶ is —CH₃, R⁷ is H, R⁸ is H, X³ is CR⁹ and R⁹ is —(CR^{g}R^{h})—O—(CR^{g}R^{h})R¹²;
R² is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R³ is selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl and C₁-C₆ alkoxy;
R¹ is selected from the group consisting of -L¹-L²-L³-Cyc¹, C₁-C₆ alkyl, C₁-C₆ aminoalkyl, C₁-C₆ cyanoalkyl, C₁-C₆ alkyl-C(═O)—N(R^{a})R^{b}, —N(R^{a})R^{b}, —N(R^{a})(C₁-C₆ alkoxy-C₁-C₆ alkyl), —N(R^{a})(C₁-C₆ alkyl-C(═O)—C₁-C₆ alkyl), —N(R^{a})(C(═O)—C₁-C₆ alkyl), —N(R^{a})(C₁-C₆ alkyl-C(═O)—N(R^{c})R^{d}), —N(R^{a})(C₁-C₆ alkyl-SO₂—N(R^{c})R^{d}), —O—C₁-C₆ hydroxyalkyl, —O—(C₁-C₆ alkoxy-C₁-C₆ alkyl);
L¹, L² and L³ are each either absent or independently selected from the group consisting of O, NR^{a}, C(═O) and C₁-C₃ alkylene;
Cyc¹ is selected from the group consisting of C₆-C₁₀ aryl, 5-10 membered heteroaryl, C₃-C₈cycloalkyl, 3-10 membered heterocycloalkyl, C₃-C₈ cycloalkenyl and 3-8 membered heterocycloalkenyl, each optionally substituted by one, two or three R¹⁰;
R^{a}, R^{b}, R^{c} and R^{d} are each independently selected at each occurrence from the group consisting of H and C₁-C₄ alkyl;
R⁴ and R⁵ are each independently selected from the group consisting of H and C₁-C₄ alkyl, C₁-C₄ aminoalkyl and C₁-C₄ alkoxy-C₁-C₄ alkyl, or alternatively R⁴ and R⁵ together with the carbon atom to which they are attached form a C₃-C₆ cycloalkyl optionally substituted by one R¹⁰ or a 4-8 membered heterocycloalkyl optionally substituted by one R¹⁰;
R⁶ is selected from the group consisting of H, halogen, cyano, nitro, C₁-C₆ alkyl, —C₁-C₆ fluoroalkyl, C₁-C₆ aminoalkyl, C₁-C₆ alkoxy-C₁-C₆ alkyl, —OR¹², —SR¹², C₁-C₃ alkyl-C(═O)—N(R^{e})—N(R^{f})—C(═O)—CH═CH—C(═O)—O—C₁-C₆ alkyl, C₁-C₃ alkyl-C(═O)—N(R^{e})—N(R^{f})—C(═O)—CH═CH—O—C₁-C₆ alkyl, C₁-C₃ alkyl-C(═O)—N(R^{e})—N(R^{f})—C(═O)-oxiran-C(═O)—O—C₁-C₆ alkyl, C₁-C₃ alkyl-C(═O)—N(R^{e})—N(R^{f})—C(═O)-oxiran-C(═O)—OH and C₁-C₃ alkyl-C(═O)—N(R^{e})—N(R)—C(═O)—CH═CH—C(═O)—N(R¹¹)R¹²;
R^{e} and R^{f} are each independently selected at each occurrence from the group consisting of H and C₁-C₄ alkyl;
R⁷ and R⁸ are each independently selected from the group consisting of H, halogen, hydroxy, C₁-C₆ alkyl, C₁-C₆ alkoxy-C₁-C₆ alkyl, 4-10 membered heterocycloalkyl, —C₁-C₆ alkyl-NR¹⁴R¹⁵, —NHR¹⁶, —N(R¹³)—C(═O)—R¹⁶, and —C(═O)—NR¹⁴R¹⁵, wherein any said 4-10 membered heterocycloalkyl is optionally substituted with one, two or three R¹⁰;
R⁹ is selected from the group consisting of H, halogen, hydroxy, cyano, C₁-C₆ alkyl, C₁-C₆ alkoxy-C₁-C₆ alkyl, C₁-C₆ alkyl-C(═O)—OH, —C(═O)—O—C₁-C₆ alkyl, —C(═O)—N(R¹¹)R¹², —C(═O)—N(R¹¹)—C₁-C₃ alkyl-R¹², —C(═O)—N(R¹¹)—(CR^{g}R^{h})—R¹², —(CR^{g}R^{h})—CH(OH)—(CR^{g}R^{h})—R¹², —(CR^{g}R^{h})—CH(OCH₃)—(CR^{g}R^{h})—R¹², —(CR^{g}R^{h})—N(R¹¹)—(CR^{g}R^{h})—R¹², —(CR^{g}R^{h})—O—(CR^{g}R^{h})—R¹², —(CR^{g}R^{h})—S—(CR^{g}R^{h})—R¹², —(CR^{g}R^{h})—S(═O)—(CR^{g}R^{h})—R¹², —(CR^{g}R^{h})—SO₂—(CR^{g}R^{h})—R¹², —SO₂—N(R¹¹)—(CR^{g}R^{h})—R¹², —C₁-C₃ alkyl-C(═O)—N(R¹¹)R¹², —(CR^{g}R^{h})—C(═O)—N(R¹¹)R¹², —(CR^{g}R^{h})—SO₂—N(R¹¹)R¹², —C₁-C₃ alkyl-C(═O)—N(R¹¹)—C₁-C₃ alkyl-R¹², —N(R¹¹)R¹², —N(R¹¹)—C₁-C₃ alkyl-R¹², —O—C₁-C₃ alkyl-R¹², —N(R¹¹)—C(═O)—N(R¹³)—R¹², —C₁-C₃ alkyl-N(R¹¹)R¹², —C₁-C₃ alkyl-N(R¹¹)—C₁-C₃ alkyl-R¹², —C₁-C₃ alkyl-N(R¹¹)—C(═O)—R¹², —(CR^{g}R^{h})—N(R¹¹)—(CR^{g}R^{h})—R¹², —(CR^{g}R^{h})—N(R¹¹)—C(═O)—R¹², —C₁-C₃ alkyl-N(R¹¹)—C(═O)—C₁-C₃ alkyl-R¹² and —(CR^{g}R^{h})—OR¹²;
Each of R^{g} and R^{h} is independently selected from the group consisting of H, cyano, halogen, hydroxy, C₁-C₆ alkyl, C₁-C₆ fluoroalkyl, C₁-C₆ aminoalkyl, C₁-C₆ alkoxy, C₁-C₆ hydroxyalkyl, —C₁-C₆ alkoxy-C₁-C₆ alkyl, or alternatively, R^{g} and R^{h} together with the carbon atom to which they are attached form a C₁-C₆ cycloalkyl or a 4-8 membered Heterocycloalkyl, each optionally substituted by one R¹⁰;
with the proviso that at least one of R⁷, R⁸ and R⁹ is H and that R⁶, R⁷, R⁸ and R⁹ are not all H at the same time;
Each R¹⁰ is independently selected at each occurrence from the group consisting of cyano, nitro, oxo, 1-(1-cyanocyclopropyl)methyl, halogen, C₁-C₆ alkyl, C₁-C₆ fluoroalkyl, C₁-C₆ aminoalkyl, —C₁-C₆ alkyl-R¹², C₁-C₆ alkoxy, C₁-C₆ alkoxy-C₁-C₆ alkyl, C₂-C₆ alkenyl, C₂-C₆ alkynyl, C₃-C₆ cycloalkyl, 3-8 membered heterocycloalkyl, —COOH, —C(═O)—O—C₁-C₆ alkyl, —C(═O)—N(R¹¹)R¹², —N(R¹¹)R¹², —C₁-C₃ alkyl-N(R¹¹)R¹², —C₁-C₃ alkyl-C(═O)—N(R¹¹)R¹², —(C(CH₃)₂)—C(═O)—N(R¹¹)R¹², —N(R¹¹)C(═O)R¹², —C₁-C₃ alkyl-N(R¹¹)C(═O)R¹², —S(O)R¹², —S(O)₂R¹², —C₁-C₃ alkyl-S(O)₂R¹², —S(O)₂N(R¹¹)R¹², —N(R¹³)S(O)₂N(R¹¹)R¹², —N(R¹³)S(O)₂R¹², —OR¹², —C₁-C₃ alkyl-OR¹² and —SR¹²;
Each R¹¹ is independently selected from H, —SO₂CH₃, —C(═O)CH₃ and C₁-C₆ alkyl;
Each R¹² is independently selected from H, C₁-C₆ alkyl, C₁-C₆ alkoxy-C₁-C₆ alkyl, C₁-C₆ aminoalkyl, di(C₁-C₃ alkyl)amino-C₁-C₆ alkyl, C₁-C₆ fluoroalkyl, C₃-C₈ cycloalkyl, 4-8 membered heterocycloalkyl, phenyl and 5-6 membered heteroaryl, wherein said C₃-C₈ cycloalkyl, 4-8 membered heterocycloalkyl, phenyl and 5-6 membered heteroaryl are each optionally substituted by one or two R¹⁷;
Or alternatively R¹¹ and R¹² together form a 4-8 membered heterocycloalkyl;
Each R¹³ is independently selected from H and C₁-C₆ alkyl;
Each R¹⁴ and R¹⁵ is independently selected from the group consisting of H, C₁-C₆ alkyl, C₁-C₆ fluoroalkyl, C₁-C₆ aminoalkyl, —SO₂—C₁-C₆ alkyl and C₁-C₆ alkoxy-C₁-C₆ alkyl;
Each R¹⁶ is a 3-8 membered heterocycloalkyl optionally substituted with one, two or three R¹⁰; and
Each R¹⁷ is independently selected from the group consisting of H, halogen, hydroxy, cyano, C₁-C₆ alkyl, C₁-C₆ hydroxyalkyl, C₁-C₆ alkoxy-C₁-C₆ alkyl, C₁-C₆ aminoalkyl, C₁-C₆ fluoroalkyl, C₁-C₆ alkoxy and C₃-C₆ cycloalkyl.

2. A compound of claim 1 having the formula (Ib1): 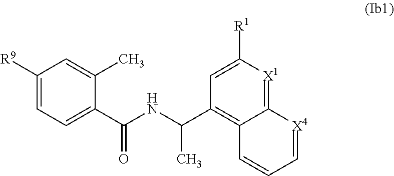
or a pharmaceutically acceptable salt thereof, wherein X¹ is CH or N and X⁴ is CH or X¹ is CH and X⁴ is N.

3. A compound of claim 2 which is of formula: 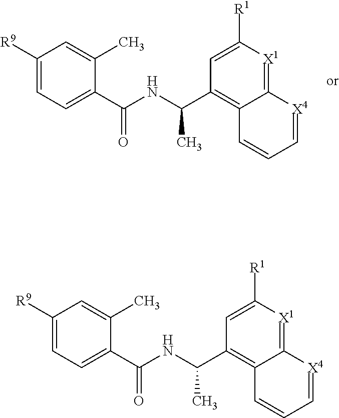
or a pharmaceutically acceptable salt thereof.

4. A compound of claim 1 having Formula (Ib2): 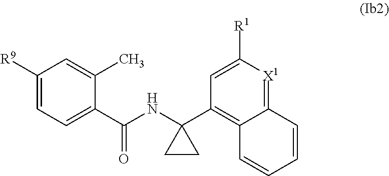
or a pharmaceutically acceptable salt thereof, wherein X¹ is CH or N.

5. A compound of claim 1 having Formula (Ib3): 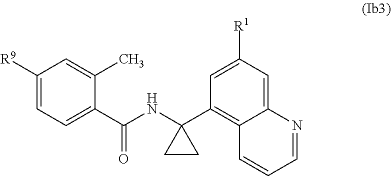
or a pharmaceutically acceptable salt thereof, wherein R⁹ is —(CR^{g}R^{h})—O—(CR^{g}R^{h})R¹².

6. A compound of any one of claims 2 to 4, or a pharmaceutically acceptable salt thereof, wherein:
R⁹ is selected from the group consisting of C₁-C₆ alkyl-C(═O)—OH, —C(═O)—N(R¹¹)R¹², —C(═O)—N(R¹¹)—C₁-C₃ alkyl-R¹², —C(═O)—N(R¹¹)—(CR^{g}R^{h})—R¹²—(CR^{g}R^{h})—CH(OH)—(CR^{g}R^{h})—R¹², —(CR^{g}R^{h})—CH(OCH₃)—(CR^{g}R^{h})—R¹², —(CR^{g}R^{h})—N(R¹¹)—(CR^{g}R^{h})—R¹², —(CR^{g}R^{h})—O—(CR^{g}R^{h})—R¹², (CR^{g}R^{h})—S—(CR^{g}R^{h})—R¹², —(CR^{g}R^{h})—S(═O)—(CR^{g}R^{h})—R¹², —C₁-C₃ alkyl-C(═O)—N(R¹¹)R¹², —(CR^{g}R^{h})—C(═O)—N(R¹¹)R¹², —C₁-C₃ alkyl-N(R¹¹)R¹², —C₁-C₃ alkyl-N(R¹¹)—C₁-C₃ alkyl-R¹², —(CR^{g}R^{h})—N(R¹¹)—C(═O)—R¹² and —(CR^{g}R^{h})—OR¹²;
Each of R^{g} and R^{h} is independently selected from the group consisting of H, C₁-C₆ alkyl, C₁-C₆ fluoroalkyl, —C₁-C₆ alkoxy-C₁-C₆ alkyl, or alternatively, R^{g} and R^{h} together with the carbon atom to which they are attached form a C₃-C₆ cycloalkyl;
Each R¹¹ is independently selected from H, —SO₂CH₃, —C(═O)CH₃ and C₁-C₆ alkyl;
Each R¹² is independently selected from C₁-C₆ alkyl, C₃-C₈ cycloalkyl, 4-8 membered heterocycloalkyl and 5-6 membered heteroaryl, wherein said C₃-C₈ cycloalkyl, 4-8 membered heterocycloalkyl and 5-9 membered heteroaryl are each optionally substituted by one or two R¹⁷; and
Each R¹⁷ is independently selected from the group consisting of halogen, C₁-C₆ alkyl, C₁-C₆ hydroxyalkyl, C₁-C₆ fluoroalkyl and C₃-C₆ cycloalkyl.

7. A compound of claim 6, or a pharmaceutically acceptable salt thereof, wherein R⁹ is —CH₂—C(═O)OH, —CH₂NH₂, —CH₂—C(═O)—NH(CH₃), —CH₂—C(═O)—N(CH₃)₂, —CH₂—NH—CH₂-heteroaryl or —CH₂—NH—C(═O)-heteroaryl, —C(═O)—NH—CH₂-heteroaryl, —C(═O)—NH—CH(CH₃)-heteroaryl, —C(═O)—NH—C(CH₃)₂-heteroaryl, —CH₂—C(═O)—NH-heteroaryl, —C(═O)—NH—C(cyclopropyl)-heteroaryl, —C(CH₃)₂—NH—CH₂-heteroaryl, —CH₂—N(CH₃)—CH₂-heteroaryl, —CH₂—N(CH₃)—C(═O)-heteroaryl, —CH(CH₃—)—NH—C(═O)-heteroaryl, —C(cyclopropyl)-NH—C(═O)-heteroaryl, —C(═O)—N(CH₃)—CH₂-heteroaryl, —CH₂—N(SO₂CH₃)—CH₂-heteroaryl, —CH₂—N(C(O)CH₃)—CH₂-heteroaryl, —C(CH₃)₂—NH—C(═O)-heteroaryl, —CH₂—O—CH₂-heteroaryl, —CH₂—S—CH₂-heteroaryl, —CH₂—S(O)—CH₂-heteroaryl, —CH(CH₃)—O—CH₂-heteroaryl, —CH(OH)—C(═O)—NH-heteroaryl, —CH(CF₃)—NH—CH₂-heteroaryl, —CH(CH₂OCH₃)—O—CH₂-heteroaryl, —CH(CH₂OCH₃)—NH—CH₂-heteroaryl, —CH₂—CH(OH)— CH₂-heteroaryl, —CH₂—CH(OCH₃)—CH₂-heteroaryl, —CH₂—O-heteroaryl, wherein said heteroaryl is a pyridinyl or a 5-membered heteroaryl comprising 1 N atom and additionally comprising 1, 2, or 3 heteroatoms selected from N, O and S, said heteroaryl being optionally substituted by one or two R¹⁷ groups selected from methyl, ethyl, isopropyl, hydroxymethyl, chloro, difluoromethyl, trifluoromethyl, cyclopropyl, cyclobutyl or cyclopentyl.

8. A compound of any one of claims 2 to 7, or a pharmaceutically acceptable salt thereof, wherein R⁹ is —CH₂—O—CH₂-heteroaryl and said heteroaryl is a pyridinyl or a 5-membered heteroaryl comprising 1 N atom and additionally comprising 1, 2, or 3 heteroatoms selected from N, O and S.

9. A compound of claim 4, or a pharmaceutically acceptable salt thereof, wherein X¹ is N; R⁹ is —CH₂—O—CH₂-heteroaryl wherein said heteroaryl is pyridinyl, thiazolyl or oxazolyl; and R¹ is a pyrazolyl substituted by —C(═O)—N(CH₃)₂ or —CH₂—C(═O)—N(CH₃)₂.

10. A compound of claim 5, or a pharmaceutically acceptable salt thereof, wherein R⁹ is —CH₂—O—CH₂-heteroaryl wherein said heteroaryl is pyridinyl, thiazolyl or oxazolyl; and R¹ is a pyrazolyl substituted by —C(═O)—N(CH₃)₂ or —CH₂—C(═O)—N(CH₃)₂.

11. A compound of any one of claims 9 and 10, or a pharmaceutically acceptable salt thereof, wherein R⁹ is selected from:

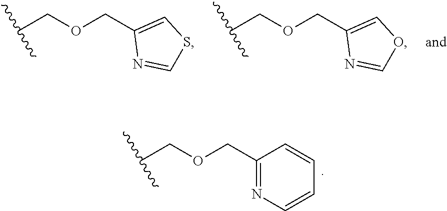

12. A compound of any one of claims 9 to 11, or a pharmaceutically acceptable salt thereof, wherein R¹ is:

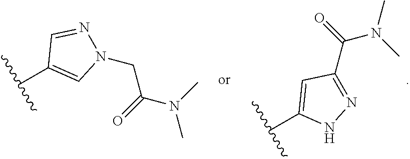 [SMILES: *c1cnn(CC(=O)N(C)C)c1 ; *c1cc(C(=O)N(C)C)n[nH]1]

13. A compound of claim 1 which is selected from the group consisting of:
4-((((1H-imidazol-2-yl)methyl)amino)methyl)-2-methyl-N-(1-(3-(1-methyl-1H-pyrazol-4-yl)naphthalen-1-yl)ethyl)benzamide;
2-methyl-N-(1-(3-(1-methyl-1H-pyrazol-4-yl)naphthalen-1-yl)ethyl)-4-(((thiazol-4-ylmethyl)amino)methyl)benzamide;
(R)-2-methyl-N-(1-(2-(1-methyl-1H-pyrazol-4-yl)quinolin-4-yl)ethyl)-4-(((thiazol-4-ylmethyl)amino)methyl)benzamide;
N-(3-methyl-4-((1-(3-(1-methyl-1H-pyrazol-4-yl)naphthalen-1-yl)ethyl)carbamoyl)benzyl)thiazole-4-carboxamide N-(3-methyl-4-((1-(3-(1-methyl-1H-pyrazol-4-yl)naphthalen-1-yl)ethyl)carbamoyl)benzyl)-1H-imidazole-2-carboxamide;
(R)-2-(3-methyl-4-((1-(2-(1-methyl-1H-pyrazol-4-yl)quinolin-4-yl)ethyl)carbamoyl)phenyl)acetic acid;
(R)-2-methyl-N1-(1-(2-(1-methyl-1H-pyrazol-4-yl)quinolin-4-yl)ethyl)-N4-(thiazol-4-ylmethyl)terephthalamide;
2-methyl-N1-((R)-1-(2-(1-methyl-1H-pyrazol-4-yl)quinolin-4-yl)ethyl)-N4-(1-(thiazol-4-yl)ethyl)terephthalamide;
(R)-2-methyl-N1-(1-(2-(1-methyl-1H-pyrazol-4-yl)quinolin-4-yl)ethyl)-N4-((5-methylthiazol-4-yl)methyl)terephthalamide;
(R)-2-methyl-N1-(1-(2-(1-methyl-1H-pyrazol-4-yl)quinolin-4-yl)ethyl)-N4-((2-methylthiazol-4-yl)methyl)terephthalamide;
(R)-N4-(1-(1H-imidazol-2-yl)cyclopropyl)-2-methyl-N1-(1-(2-(1-methyl-1H-pyrazol-4-yl)quinolin-4-yl)ethyl)terephthalamide;
(R)—N4,2-dimethyl-N¹-(1-(2-(1-methyl-1H-pyrazol-4-yl)quinolin-4-yl)ethyl)-N⁴-(thiazol-4-ylmethyl)terephthalamide;
(R)-N4-(2-(1H-imidazol-2-yl)propan-2-yl)-2-methyl-N1-(1-(2-(1-methyl-1H-pyrazol-4-yl)quinolin-4-yl)ethyl)terephthalamide;
(R)-N4-((1H-imidazol-2-yl)methyl)-2-methyl-N1-(1-(2-(1-methyl-1H-pyrazol-4-yl)quinolin-4-yl)ethyl)terephthalamide;
N4-(1-(1H-imidazol-2-yl)ethyl)-2-methyl-N1-((R)-1-(2-(1-methyl-1H-pyrazol-4-yl)quinolin-4-yl)ethyl)terephthalamide;
2-methyl-N⁴-((1-methyl-1H-imidazol-2-yl)methyl)-N¹-(1-(2-(1-methyl-1H-pyrazol-4-yl)quinolin-4-yl)cyclopropyl) terephthalamide;
2-methyl-N¹-(1-(2-(1-methyl-1H-pyrazol-4-yl)quinolin-4-yl)cyclopropyl)-N⁴-(thiazol-4-ylmethyl)terephthalamide;
(R)-2-methyl-N4-((1-methyl-1H-imidazol-2-yl)methyl)-N¹-(1-(2-(1-methyl-1H-pyrazol-4-yl)quinolin-4-yl)ethyl)terephthalamide;
2-methyl-N-(1-(2-(1-methyl-1H-pyrazol-4-yl)quinolin-4-yl)cyclopropyl)-4-(((thiazol-4-ylmethyl)amino)methyl)benzamide;
(R)—N-(1-(3-methyl-4-((1-(2-(1-methyl-1H-pyrazol-4-yl)quinolin-4-yl)ethyl) carbamoyl)phenyl)cyclopropyl)thiazole-4-carboxamide;
(R)—N-(1-(3-methyl-4-((1-(2-(1-methyl-1H-pyrazol-4-yl)quinolin-4-yl)ethyl)carbamoyl)phenyl)cyclopropyl)-1H-imidazole-2-carboxamide;
(R)—N-(2-(3-methyl-4-((1-(2-(1-methyl-1H-pyrazol-4-yl)quinolin-4-yl)ethyl) carbamoyl)phenyl)propan-2-yl)thiazole-4-carboxamide;
(R)—N-(2-(3-methyl-4-((1-(2-(1-methyl-1H-pyrazol-4-yl)quinolin-4-yl)ethyl) carbamoyl)phenyl)propan-2-yl)-1H-imidazole-2-carboxamide;
(R)-2-methyl-N-(1-(2-(1-methyl-1H-pyrazol-4-yl)quinolin-4-yl)ethyl)-4-(2-((thiazol-4-ylmethyl)amino)propan-2-yl)benzamide;
N-(1-(3-methyl-4-(((R)-1-(2-(1-methyl-1H-pyrazol-4-yl)quinolin-4-yl)ethyl)carbamoyl)phenyl)ethyl)thiazole-4-carboxamide;
N-(1-(3-methyl-4-(((R)-1-(2-(1-methyl-1H-pyrazol-4-yl)quinolin-4-yl)ethyl)carbamoyl)phenyl)ethyl)-1H-imidazole-2-carboxamide;
(R)-2-methyl-4-((methyl(thiazol-4-ylmethyl)amino)methyl)-N-(1-(2-(1-methyl-1H-pyrazol-4-yl)quinolin-4-yl)ethyl)benzamide;
(R)-2-methyl-4-((((1-methyl-1H-imidazol-5-yl)methyl)amino)methyl)-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-(2-oxo-2-(thiazol-4-ylamino)ethyl)benzamide;
(R)-2-methyl-N-(1-(2-(1-methyl-1H-pyrazol-4-yl)quinolin-4-yl)ethyl)-4-(2-oxo-2-(thiazol-5-ylamino)ethyl)benzamide;
(R)-2-methyl-N-(1-(2-(1-methyl-1H-pyrazol-4-yl)quinolin-4-yl)ethyl)-4-(2-oxo-2-(thiazol-2-ylamino)ethyl)benzamide;
(R)-4-(2-((1H-imidazol-2-yl)amino)-2-oxoethyl)-2-methyl-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-(2-(methylamino)-2-oxoethyl)benzamide;
(R)-4-(2-(dimethylamino)-2-oxoethyl)-2-methyl-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-((N-(thiazol-4-ylmethyl)acetamido)methyl)benzamide;
(R)-2-methyl-N-(1-(2-(1-methyl-1H-pyrazol-4-yl)quinolin-4-yl)ethyl)-4-((N-(thiazol-4-ylmethyl)methylsulfonamido)methyl)benzamide;
(R)—N-methyl-N-(3-methyl-4-((1-(2-(1-methyl-1H-pyrazol-4-yl)quinolin-4-yl)ethyl)carbamoyl)benzyl)thiazole-4-carboxamide;
(R)—N-methyl-N-(3-methyl-4-((1-(2-(1-methyl-1H-pyrazol-4-yl)quinolin-4-yl)ethyl)carbamoyl)benzyl)-1H-imidazole-2-carboxamide;
(R)-2-methyl-N-(1-(2-(1-methyl-1H-pyrazol-4-yl)quinolin-4-yl)ethyl)-4-((thiazol-4-ylmethoxy)methyl)benzamide;
(R)-2-methyl-4-((((1-methyl-1H-imidazol-2-yl)methyl)thio)methyl)-N-(1-(2-(1-methyl-1H-pyrazol-4-yl)quinolin-4-yl)ethyl)benzamide;
2-methyl-4-((((1-methyl-1H-imidazol-2-yl)methyl)sulfinyl)methyl)-N—((R)-1-(2-(1-methyl-1H-pyrazol-4-yl)quinolin-4-yl)ethyl)benzamide;
4-methyl-N—((R)-1-(2-(1-methyl-1H-pyrazol-4-yl)quinolin-4-yl)ethyl)-6-(1-(thiazol-4-ylmethoxy)ethyl)nicotinamide;
(R)-2-methyl-4-(((1-methyl-1H-imidazol-2-yl)methoxy)methyl)-N-(1-(2-(1-methyl-1H-pyrazol-4-yl)quinolin-4-yl)ethyl)benzamide;
(R)-2-methyl-N-(1-(2-(1-methyl-1H-pyrazol-4-yl)quinolin-4-yl)ethyl)-4-(2-((5-methylthiazol-4-yl)amino)-2-oxoethyl)benzamide;
4-(1-hydroxy-2-oxo-2-(thiazol-4-ylamino)ethyl)-2-methyl-N—((R)-1-(2-(1-methyl-1H-pyrazol-4-yl)quinolin-4-yl)ethyl)benzamide;
4-methyl-6-(1-((1-methyl-1H-imidazol-2-yl)methoxy)ethyl)-N—((R)-1-(2-(1-methyl-1H-pyrazol-4-yl)quinolin-4-yl)ethyl)nicotinamide;
2-methyl-N-(1-(2-(1-methyl-1H-pyrazol-4-yl)quinolin-4-yl)cyclopropyl)-4-((thiazol-4-ylmethoxy)methyl)benzamide;
2-methyl-N—((R)-1-(2-(1-methyl-1H-pyrazol-4-yl)quinolin-4-yl)ethyl)-4-(2,2,2-trifluoro-1-((thiazol-4-ylmethyl)amino)ethyl)benzamide;
2-methyl-N-(1-(2-(1-methyl-1H-pyrazol-4-yl)quinolin-4-yl)cyclopropyl)-4-(2-oxo-2-(thiazol-4-ylamino)ethyl)benzamide;
(R)-2-methyl-N1-(1-(2-(1-methyl-1H-pyrazol-4-yl)quinolin-4-yl)ethyl)-N4-(1-(thiazol-4-yl)cyclopropyl)terephthalamide;
(R)-2-methyl-N1-(1-(2-(1-methyl-1H-pyrazol-4-yl)quinolin-4-yl)ethyl)-N4-((5-methyloxazol-4-yl)methyl)terephthalamide;
rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-1;
rel-(R)-4-(2-methoxy-1-(thiazol-4-ylmethoxy)ethyl)-2-methyl-N-(1-(2-(1-methyl-1H-pyrazol-4-yl)quinolin-4-yl)cyclopropyl)benzamide, ENT-2;
4-(2-methoxy-3-(pyridin-2-yl)propyl)-2-methyl-N-(1-(2-(1-methyl-1H-pyrazol-4-yl)quinolin-4-yl)cyclopropyl)benzamide;
4-(2-hydroxy-3-(pyridin-2-yl)propyl)-2-methyl-N-(1-(2-(1-methyl-1H-pyrazol-4-yl)quinolin-4-yl)cyclopropyl)benzamide;
2-methyl-N-(1-(2-(1-(2-(methylsulfonyl)ethyl)-1H-pyrazol-4-yl)quinolin-4-yl)cyclopropyl)-4-((thiazol-4-ylmethoxy)methyl)benzamide;
N-(2-(dimethylamino)ethyl)-N-methyl-5-(4-(1-(2-methyl-4-((thiazol-4-ylmethoxy)methyl)benzamido)cyclopropyl)quinolin-2-yl)-1H-pyrazole-3-carboxamide;
4-(((5-(hydroxymethyl)thiazol-4-yl)methoxy)methyl)-2-methyl-N-(1-(2-(1-methyl-1H-pyrazol-4-yl)quinolin-4-yl)cyclopropyl)benzamide;
N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide;
N-(1-(2-(1-(2-amino-2-oxoethyl)-1H-pyrazol-4-yl)quinolin-4-yl)cyclopropyl)-2-methyl-4-((thiazol-4-ylmethoxy)methyl)benzamide;
4-(2-methoxy-1-(thiazol-4-ylmethoxy)ethyl)-2-methyl-N-(1-(2-methyl-7-(1-methyl-1H-pyrazol-4-yl)quinolin-5-yl)cyclopropyl)benzamide;
N-(1-(2-(2-hydroxypropan-2-yl)-7-(1-methyl-1H-pyrazol-4-yl)quinolin-5-yl)cyclopropyl)-2-methyl-4-((thiazol-4-ylmethoxy)methyl)benzamide;
N-(1-(2-hydroxy-7-(1-methyl-1H-pyrazol-4-yl)quinolin-5-yl)cyclopropyl)-2-methyl-4-((thiazol-4-ylmethoxy)methyl)benzamide;
N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide;
4-(2-methoxy-1-(thiazol-4-ylmethoxy)ethyl)-2-methyl-N—((R)-1-(2-(1-methyl-1H-pyrazol-4-yl)quinolin-4-yl)ethyl)benzamide;
4-((R*)-2-methoxy-1-(thiazol-4-ylmethoxy)ethyl)-2-methyl-N—((R)-1-(2-(1-methyl-1H-pyrazol-4-yl)quinolin-4-yl)ethyl)benzamide, ENT-1;
4-((R*)-2-methoxy-1-(thiazol-4-ylmethoxy)ethyl)-2-methyl-N—((R)-1-(2-(1-methyl-1H-pyrazol-4-yl)quinolin-4-yl)ethyl)benzamide, ENT-2;
rel-(R)-4-(2-methoxy-1-(thiazol-4-ylmethoxy)ethyl)-2-methyl-N-(1-(7-(1-methyl-1H-pyrazol-4-yl)quinolin-5-yl)cyclopropyl)benzamide, ENT-1;
rel-(R)-4-(2-methoxy-1-(thiazol-4-ylmethoxy)ethyl)-2-methyl-N-(1-(7-(1-methyl-1H-pyrazol-4-yl)quinolin-5-yl)cyclopropyl)benzamide, ENT-2;
rel-(R)-5-(4-(1-(4-(2-methoxy-1-(thiazol-4-ylmethoxy)ethyl)-2-methylbenzamido)cyclopropyl)quinolin-2-yl)-N,N-dimethyl-1H-pyrazole-3-carboxamide, ENT-1;
rel-(R)-5-(4-(1-(4-(2-methoxy-1-(thiazol-4-ylmethoxy)ethyl)-2-methylbenzamido)cyclopropyl)quinolin-2-yl)-N,N-dimethyl-1H-pyrazole-3-carboxamide, ENT-2;
rel-(R)-4-(2-methoxy-1-(oxazol-4-ylmethoxy)ethyl)-2-methyl-N-(1-(2-(1-methyl-1H-pyrazol-4-yl)quinolin-4-yl)cyclopropyl)benzamide, ENT-1;
rel-(R)-4-(2-methoxy-1-(oxazol-4-ylmethoxy)ethyl)-2-methyl-N-(1-(2-(1-methyl-1H-pyrazol-4-yl)quinolin-4-yl)cyclo-propyl)benzamide, ENT-2;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclopropyl)benzamide, ENT-1;
2-methyl-N-(1-(2-(1-((methylsulfonyl)methyl)-1H-pyrazol-4-yl)quinolin-4-yl)cyclopropyl)-4-((thiazol-4-ylmethoxy)methyl)benzamide;
N,N-dimethyl-5-(4-(1-(2-methyl-4-((thiazol-4-ylmethoxy)methyl)benzamido)cyclopropyl)quinolin-2-yl)-1H-pyrazole-3-carboxamide;
2-methyl-N-(1-(2-(1-methyl-1H-pyrazol-4-yl)quinolin-4-yl)cyclopropyl)-4-((oxazol-4-ylmethoxy)methyl)benzamide;
2-methyl-N-(1-(7-(1-methyl-1H-pyrazol-4-yl)quinolin-5-yl)cyclopropyl)-4-((oxazol-4-ylmethoxy)methyl)benzamide;
N,N-dimethyl-5-(4-(1-(2-methyl-4-((oxazol-4-ylmethoxy)methyl)benzamido)cyclopropyl)quinolin-2-yl)-1H-pyrazole-3-carboxamide;
N-methyl-5-(4-(1-(2-methyl-4-((thiazol-4-ylmethoxy)methyl)benzamido)cyclopropyl)quinolin-2-yl)-1H-pyrazole-3-carboxamide;
2-methyl-N-(1-(2-methyl-7-(1-methyl-1H-pyrazol-4-yl)quinolin-5-yl)cyclopropyl)-4-((oxazol-4-ylmethoxy)methyl)benzamide;
5-(4-(1-(2-methyl-4-((thiazol-4-ylmethoxy)methyl)benzamido)cyclo-propyl)quinolin-2-yl)-1H-pyrazole-3-carboxamide;
N,N-dimethyl-5-(5-(1-(2-methyl-4-((oxazol-4-ylmethoxy)methyl)benzamido)cyclopropyl) quinolin-7-yl)-1H-pyrazole-3-carboxamide;
N,N-dimethyl-5-(5-(1-(2-methyl-4-((thiazol-4-ylmethoxy)methyl)benzamido)cyclopropyl)quinolin-7-yl)-1H-pyrazole-3-carboxamide;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclo-propyl)benzamide, ENT-1;
rel-(R)-4-(2-methoxy-1-(oxazol-4-ylmethoxy)ethyl)-2-methyl-N-(1-(7-(1-methyl-1H-pyrazol-4-yl)quinolin-5-yl)cyclo-propyl)benzamide, ENT-2;
N-(2-hydroxy-2-methylpropyl)-N-methyl-5-(4-(1-(2-methyl-4-((thiazol-4-ylmethoxy)methyl)benzamido)cyclopropyl) quinolin-2-yl)-1H-pyrazole-3-carboxamide;
rel-(R)-5-(5-(1-(4-(2-methoxy-1-(oxazol-4-ylmethoxy)ethyl)-2-methylbenzamido)cyclopropyl)quinolin-7-yl)-N,N-dimethyl-1H-pyrazole-3-carboxamide, ENT-1;
rel-(R)-5-(5-(1-(4-(2-methoxy-1-(oxazol-4-ylmethoxy)ethyl)-2-methylbenzamido)cyclopropyl)quinolin-7-yl)-N,N-dimethyl-1H-pyrazole-3-carboxamide, ENT-1;
rel-(R)-5-(5-(1-(4-(2-methoxy-1-(thiazol-4-ylmethoxy)ethyl)-2-methylbenzamido)cyclopropyl)quinolin-7-yl)-N,N-dimethyl-1H-pyrazole-3-carboxamide, ENT-1;
N,N-dimethyl3-(4-{4-[1-(4-{[(1,3-thiazol-4-yl)methoxy]methyl}-2-toluoylamino)cyclopropyl]-2-quinolyl}-1-pyrazolyl)propionamide;
N,N-dimethyl2-methyl-2-(4-{4-[1-(4-{[(1,3-thiazol-4-yl)methoxy]methyl}-2-toluoylamino)cyclopropyl]-2-quinolyl}-1-pyrazolyl)propionamide;
N-ethyl-N-methyl(4-{4-[1-(4-{[(1,3-thiazol-4-yl)methoxy]methyl}-2-toluoylamino)cyclopropyl]-2-quinolyl}-1-pyrazolyl)acetamide; and
rel-(R)-4-(2-methoxy-1-(oxazol-4-ylmethoxy)ethyl)-2-methyl-N-(1-(2-methyl-7-(1-methyl-1H-pyrazol-4-yl)quinolin-5-yl)cyclopropyl)benzamide, ENT-1;
or a pharmaceutically acceptable salt thereof.

14. A compound of claim 1 which is selected from the group consisting of:
N-(1-(2-(1-(2-(dimethylamino)-2-oxoethyl)-1H-pyrazol-4-yl)quinolin-4-yl)cyclopropyl)-2-methyl-4-((thiazol-4-ylmethoxy)methyl)benzamide;
N,N-dimethyl-5-(4-(1-(2-methyl-4-((thiazol-4-ylmethoxy)methyl)benzamido)cyclopropyl) quinolin-2-yl)-1H-pyrazole-3-carboxamide;
N,N-dimethyl-5-(5-(1-(2-methyl-4-((oxazol-4-ylmethoxy)methyl)benzamido)cyclopropyl) quinolin-7-yl)-1H-pyrazole-3-carboxamide; and
N-(1-(2-(1-(2-(dimethylamino)-2-oxoethyl)-1H-pyrazol-4-yl)quinolin-4-yl)cyclopropyl)-2-methyl-4-((pyridin-2-ylmethoxy)methyl)benzamide;
or a pharmaceutically acceptable salt thereof.

15. A pharmaceutical composition comprising a compound of any one of claims 1 to 14, or a pharmaceutically acceptable salt thereof, and at least one pharmaceutically acceptable excipient.

16. A method for treating a viral infection, comprising administering to a subject in need thereof a therapeutically effective amount of a compound of any one of claims 1 to 14, or a pharmaceutically acceptable salt thereof.

17. A method for treating a viral infection of claim 16, wherein the compound of any one of claims 1 to 14, or a pharmaceutically acceptable salt thereof, is administered as a single agent.

18. A method for treating a viral infection of claim 16, further comprising administering a therapeutically effective amount of an additional therapeutic agent selected from the list consisting of viral RNA polymerase inhibitors, Mpro inhibitors, nucleoside inhibitors, host factor inhibitors, other PLpro inhibitors and metabolism boosting agents.

19. A method for treating a viral infection of any one of claims 16 to 18, wherein said viral infection is a coronavirus infection.

20. A method for treating a viral infection of claim 19, wherein said coronavirus infection is COVID-19.

21. A compound of any one of claims 1 to 14, for use as a medicament.

22. A compound of any one of claims 1 to 14, for use in the treatment of a viral infection.

23. Use of a compound of any one of claims 1 to 14 for the manufacture of a medicament for the treatment of a viral infection.

24. A method for the treatment of a disorder mediated by the papain-like protease in a subject, comprising administering to the subject in need thereof a compound of any one of claims 1 to 14, or a pharmaceutically acceptable salt thereof, in an amount that is effective for treating the disorder.
